(19) United States
(12) Patent Application Publication
(10) Pub. No.: US 2026/0050217 A1
(43) Pub. Date: Feb. 19, 2026

(54) SULFONIUM SALT MONOMER, SULFONIUM SALT QUENCHER, POLYMER, CHEMICALLY AMPLIFIED RESIST COMPOSITION, AND PATTERNING METHOD

(71) Applicant: SHIN-ETSU CHEMICAL CO., LTD., Tokyo (JP)

(72) Inventor: Masahiro Fukushima, Joetsu-shi (JP)

(73) Assignee: SHIN-ETSU CHEMICAL CO., LTD., Tokyo (JP)

(21) Appl. No.: 19/293,025

(22) Filed: Aug. 7, 2025

(30) Foreign Application Priority Data
Aug. 13, 2024 (JP) 2024-134964

(51) Int. Cl. G03F 7/039; C07C 381/12; C07D 279/20; C07D 279/22; C07D 327/06; C07D 327/08; C07D 333/46; C07D 333/54; C07D 333/76; C07D 335/02; C07D 335/12; C07D 335/16; C07D 337/04; C07D 339/08; C08F 12/32; G03F 7/00

(52) U.S. Cl. CPC G03F 7/0392; C07C 381/12; C07D 279/20; C07D 279/22; C07D 327/06; C07D 327/08; C07D 333/46; C07D 333/54; C07D 333/76; C07D 335/02; C07D 335/12; C07D 335/16; C07D 337/04; C07D 339/08; C08F 12/32; G03F 7/70033

(57) ABSTRACT

Provided are: a sulfonium salt monomer to be used as a sulfonium salt quencher, which is a material of a polymer to be contained in a chemically amplified resist composition that exhibits superior solubility in solvents and has superior lithographic performance with high sensitivity and high contrast in photolithography with use of a high-energy ray, and also has superior etching resistance with durability against pattern collapse even in fine patterning; a sulfonium salt quencher containing the sulfonium salt monomer; a polymer containing a repeating unit derived from the sulfonium salt quencher; a chemically amplified resist composition containing a base polymer containing the polymer; and a patterning method with the chemically amplified resist composition. A sulfonium salt monomer represented by the following general formula (a):

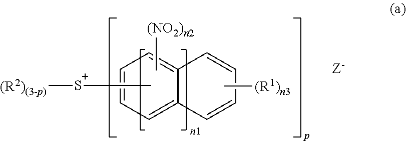

### TECHNICAL FIELD

The present invention relates to a sulfonium salt monomer, a sulfonium salt quencher, a polymer, a chemically amplified resist composition, and a patterning method.

### BACKGROUND ART

Achievement of higher integration levels and higher speed in LSIs has been accelerating miniaturization of pattern rules. In particular, the expansion of the flash memory market and the increase in memory capacity are driving such miniaturization. Mass production of 65-nm node devices has been carried out by ArF lithography as a state-of-the-art miniaturization technique, and preparations for mass production of 45-nm node devices by next-generation ArF immersion lithography are currently under way. Candidates for producing next-generation 32-nm node devices include immersion lithography with use of liquid having a higher refractive index than water and an ultra-high NA lens including a high-refractive-index lens and a high-refractive-index resist film in combination, extreme ultraviolet (EUV) lithography with a wavelength of 13.5 nm, and double exposure with ArF lithography (double-patterning lithography), and examination therefor is ongoing.

As miniaturization proceeds and the diffraction limit of light is approached, the contrast of light decreases. The reduced contrast of light causes positive-type resist films to undergo reduction in the resolution of hole patterns and trench patterns and in focus margins.

In association with the miniaturization of patterns, the line width roughness (LWR) of line patterns and the critical dimension uniformity (CDU) of hole patterns have become matters of concern. Influences of the uneven distribution or aggregation of a base polymer or an acid generator and those of acid diffusion have been pointed out. Furthermore, thinner resist films tend to have higher LWR, and the degradation of LWR due to thinning associated with the progression of miniaturization has become a serious problem.

Resist compositions for EUV lithography need to achieve high sensitivity, high resolution, and low LWR at the same time. Decreasing the acid diffusion length results in lower LWR, but in lower sensitivity. For example, lowering the post-exposure bake (PEB) temperature results in lower LWR, but in lower sensitivity. Likewise, increasing the amount of a quencher to be added results in lower LWR, but in lower sensitivity. It is needed to break down the trade-off relationship between sensitivity and LWR.

To inhibit acid diffusion, a resist compound containing a repeating unit derived from an onium salt of a sulfonic acid having a polymerizable unsaturated bond has been proposed (Patent Document 1). Such a compound, what is called a polymer-bound acid generator, is characterized by its very short acid diffusion length due to the generation of polymeric sulfonic acid through exposure. Alternatively, enhanced sensitivity is achieved by using an acid generator at a higher proportion. Also for an acid generator of addition type, increasing the amount thereof to be added results in higher sensitivity, but in this case also in an increased acid diffusion length. Because acids unevenly diffuse, increased acid diffusion leads to degradation in LWR and CDU. It can be said that polymeric acid generators have high performance in terms of the balance of sensitivity, LWR, and CDU.

Iodine atoms have been found to have an effect of generating secondary electrons therefrom during exposure because iodine atoms exhibit very large absorption of EUV at a wavelength of 13.5 nm, gathering attention in the field of EUV lithography. Patent Document 2 discloses a photo-acid generator obtained by introducing an iodine atom into an anion, and Patent Document 3 discloses a polymerizable group-containing photo-acid generator obtained by introducing an iodine atom into an anion. These have been found to contribute to improvement in lithographic performance to some degree, whereas iodine atoms do not exhibit high solubility in organic solvents, and the precipitation in solvents is a matter of concern.

To further inhibit acid diffusion, resist compositions with use of a polymer containing a repeating unit derived from a sulfonium salt of a weak acid having a polymerizable group and a pKa of −0.8 or more as a polymer-bound quencher have been proposed (Patent Documents 4 to 7). Patent Document 4 shows carboxylic acid, sulfonamide, phenol, hexafluoro alcohol, and so on as such weak acids.

### CITATION LIST

### Patent Literature

Patent Document 1: JP 4425776 B
Patent Document 2: JP 6720926 B
Patent Document 3: JP 6973274 B
Patent Document 4: WO 2019/167737 A1
Patent Document 5: WO 2022/264845 A1
Patent Document 6: JP 2022-115072 A
Patent Document 7: JP 7433394 B

### SUMMARY OF INVENTION

### Technical Problem

For chemically amplified resist compositions that are catalyzed by acids, development of a resist composition is demanded that has much higher sensitivity, enables improvement in the LWR of line patterns and the CDU of hole patterns, and is superior even in etching resistance after patterning.

The present invention has been made in view of the aforementioned circumstances, and an object of the present invention is to provide: a sulfonium salt monomer to be used as a sulfonium salt quencher, which is a material of a polymer to be contained in a chemically amplified resist composition that exhibits superior solubility in solvents and has superior lithographic performance with high sensitivity and high contrast in photolithography with use of a high-energy ray, and also has superior etching resistance with durability against pattern collapse even in fine patterning; a sulfonium salt quencher containing the sulfonium salt monomer; a polymer containing a repeating unit derived from the quencher; a chemically amplified resist composition containing a base polymer containing the polymer; and a patterning method with the chemically amplified resist composition.

### Solution to Problem

To solve the above problems, specifically, the present invention provides a sulfonium salt monomer represented by the following general formula (a):

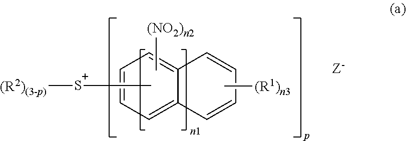

wherein p is 1, 2, or 3, n1 is 0 or 1, n2 is 1 or 2, and n3 is an integer of 0 to 6, provided that 1≤n2+n3≤5 is satisfied if n1 is 0, and 1≤n2+n3≤7 is satisfied if n1 is 1;
R¹ is a halogen atom, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein if n3 is 2 to 6, R¹ groups are identical to or different from each other, and any two R¹ groups are optionally bound together to form a ring together with the carbon atoms to which the two groups are bound;
R² is a halogen atom or a hydrocarbyl group having 1 to 30 carbon atoms and optionally containing a heteroatom, wherein if p is 1, the two R² groups are identical to or different from each other, and any two of the three substituents bound to S⁺ are optionally bound together to form a ring together with the sulfur atom to which the two substituents are bound; and
Z⁻ is a carboxylate anion having an aromatic vinyl structure and an iodine atom.

A chemically amplified resist composition produced with such a sulfonium salt monomer exhibits superior solubility in solvents, is superior in lithographic performance including exposure latitude (EL), LWR, CDU, and depth of focus (DOF) with high sensitivity and high contrast, and is superior also in etching resistance with durability against pattern collapse even in fine patterning, thus being very useful.

In this case, the above sulfonium salt monomer represented by the following general formula (a1) is preferred:

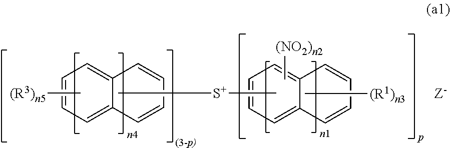

wherein p, n1 to n3, R¹, and Z⁻ are as specified above;
n4 is 0 or 1, and n5 is an integer of 0 to 5; and
R³ is a halogen atom, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein if n5 is 2 to 5, R³ groups are identical to or different from each other, and any two R³ groups are optionally bound together to form a ring together with the carbon atoms to which the two groups are bound.

The sulfonium salt monomer represented by the general formula (a1) is preferred as the above sulfonium salt monomer.

A sulfonium salt monomer in which the Z⁻ is an anion represented by the following general formula (Z) is preferred:

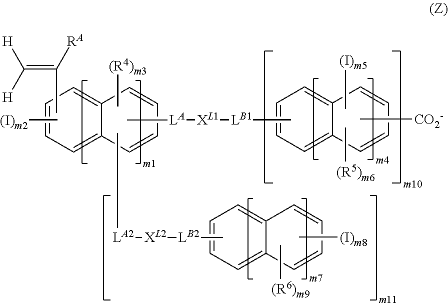

wherein m1 is 0 or 1, m2 is an integer of 0 to 4, m3 is an integer of 0 to 3, m4 is 0 or 1, m5 is an integer of 0 to 4, m6 is an integer of 0 to 3, m7 is 0 or 1, m8 is an integer of 0 to 4, m9 is an integer of 0 to 3, m10 is 0 or 1, and m11 is 0 or 1, provided that 0≤m2+m3+m11≤4 is satisfied if m1 is 0, and 0≤m2+m3+m11≤6 is satisfied if m1 is 1, that 0≤m5+m6≤4 is satisfied if m4 is 0, and 0≤m5+m6≤6 is satisfied if m4 is 1, that 0≤m8+m9≤5 is satisfied if m7 is 0, and 0≤m8+m9≤7 is satisfied if m7 is 1, and that 1≤m2+m5+m8≤4 is satisfied;
R^{A} is a hydrogen atom, a fluorine atom, a methyl group, or a trifluoromethyl group;
R⁴, R⁵, and R⁶ are each a halogen atom being not an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein: if m3 is 2 or 3, R⁴ groups are identical to or different from each other, and any two R⁴ groups are optionally bound together to form a ring together with the carbon atoms to which the two groups are bound; if m6 is 2 or 3, R⁵ groups are identical to or different from each other, and any two R⁵ groups are optionally bound together to form a ring together with the carbon atoms to which the two groups are bound; and if m9 is 2 or 3, R⁶ groups are identical to or different from each other, and any two R⁶ groups are optionally bound together to form a ring together with the carbon atoms to which the two groups are bound;
L^{A1}, L^{A2}, L^{B1}, and L^{B2} are each independently a single bond, an ether bond, a carbonyl group, an ester bond, a sulfonate bond, an amide bond, a sulfonamide bond, a carbonate bond, or a carbamate bond; and
X^{L1} and X^{L2} are each independently a single bond or a hydrocarbylene group having 1 to 40 carbon atoms and optionally containing a heteroatom.

The above sulfonium salt monomer preferably contains such an anion.

The present invention also provides a sulfonium salt quencher containing the sulfonium salt monomer.

A chemically amplified resist composition containing such a sulfonium salt quencher is preferred.

The present invention also provides a polymer containing a repeating unit derived from the sulfonium salt quencher.

A chemically amplified resist composition containing such a polymer is preferred.

The polymer preferably further contains a repeating unit or repeating units represented by either one or both of the following general formulae (b1) and (b2):

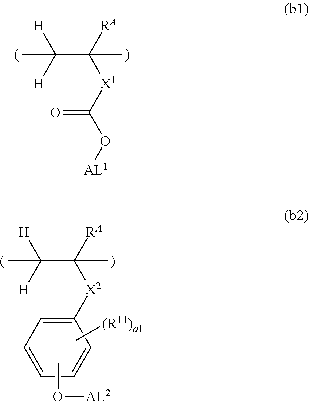

wherein R^{A} groups are each independently a hydrogen atom, a fluorine atom, a methyl group, or a trifluoromethyl group;
X¹ is a single bond, a phenylene group, a naphthylene group, *—C(═O)—O—X¹¹—, or *—C(═O)—NH—X¹¹—, wherein the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring;
X² is a single bond, *—C(═O)—O—, or *—C(═O)—NH—;
* indicates bonding to a carbon atom of a main chain;
R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom, wherein if a1 is 2 or more, R¹¹ groups are identical to or different from each other;
AL¹ and AL² are each independently an acid-unstable group; and
a1 is an integer of 0 to 4.

A chemically amplified resist composition containing such a polymer is preferred.

The polymer preferably further contains a repeating unit represented by the following general formula (b3):

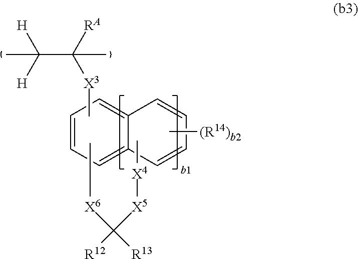

wherein b1 is 0 or 1, and b2 is an integer of 0 to 3 if b1 is 0, and an integer of 0 to 5 if b1 is 1;
R^{A} is a hydrogen atom, a fluorine atom, a methyl group, or a trifluoromethyl group;
X³ is a single bond, *—C(═O)—O—, or *—C(═O)—NH—, wherein * indicates bonding to a carbon atom of a main chain;
R¹² and R¹³ are each independently a hydrogen atom or a hydrocarbyl group having 1 to 20 carbon atoms and optionally containing a heteroatom, wherein R¹² and R¹³ are optionally bound together to form a ring together with the carbon atom to which the groups are bound;
R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A}) (R^{14B}) wherein R^{14A} and R^{14B} are each independently a hydrogen atom or a hydrocarbyl group having 1 to 6 carbon atoms, and if b2 is 2 or more, R¹⁴ groups are identical to or different from each other, and any multiple R¹⁴ groups are optionally bound together to form a ring together with the carbon atoms of the aromatic ring or aromatic rings to which the groups are bound;
X⁴ is a single bond, an aliphatic hydrocarbylene group having 1 to 4 carbon atoms, a carbonyl group, a sulfonyl group, or a group given by combining any of these; and
X⁵ and X⁶ are each independently an oxygen atom or a sulfur atom, provided that X⁴ and X⁶ are bound to adjacent carbon atoms in the aromatic ring or aromatic rings.

A chemically amplified resist composition further containing such a polymer is preferred.

The polymer preferably further contains a repeating unit represented by the following general formula (c):

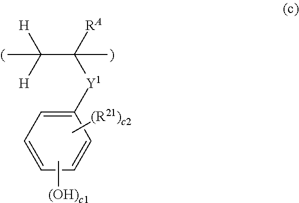

wherein R^{A} is a hydrogen atom, a fluorine atom, a methyl group, or a trifluoromethyl group;
Y¹ is a single bond, *—C(═O)—O—, or *—C(═O)—NH—, wherein * indicates bonding to a carbon atom of a main chain;
R²¹ is a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
c1 is an integer of 1 to 4, and c2 is an integer of 0 to 3, provided that 1≤c1+c2≤5 is satisfied.

A chemically amplified resist composition further containing such a polymer is preferred.

The polymer preferably further contains a repeating unit derived from an onium salt monomer containing a fluorosulfonate anion having a polymerizable group and at least one iodine atom and a sulfonium cation.

A chemically amplified resist composition further containing such a polymer is preferred.

The polymer preferably further contains a repeating unit represented by the following general formula (e):

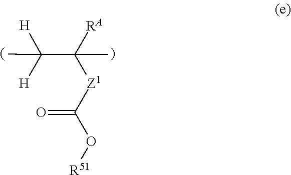

wherein R^{A} is a hydrogen atom, a fluorine atom, a methyl group, or a trifluoromethyl group;
Z¹ is a single bond, a phenylene group, a naphthylene group, *—C(═O)—O—Z¹¹—, or *—C(═O)—NH—Z¹¹—, wherein the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, * indicates bonding to a carbon atom of a main chain, Z¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring; and
R⁵¹ is a hydrogen atom or a group having 1 to 20 carbon atoms and containing at least one structure selected from a hydroxy group being not a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—).

A chemically amplified resist composition further containing such a polymer is preferred.

The present invention also provides a chemically amplified resist composition containing (A) a base polymer containing the polymer.

A chemically amplified resist composition containing a base polymer containing the polymer as component (A) is preferred.

The chemically amplified resist composition preferably further contains one or more selected from (B) an organic solvent, (C) a quencher other than the sulfonium salt quencher, (D) an acid generator, and (E) a surfactant.

A chemically amplified resist composition containing such a component is preferred.

The present invention also provides a patterning method including: a step of forming a resist film on a substrate by using the chemically amplified resist composition; a step of exposing the resist film to a high-energy ray; and a step of developing the exposed resist film by using a developer.

A resist film formed by using such a patterning method has good solubility in solvents, and is characterized by small acid diffusion because of the large atomic weight of iodine atoms.

In the patterning method, ArF excimer laser light having a wavelength of 193 nm, KrF excimer laser light having a wavelength of 248 nm, an electron beam, or an extreme ultraviolet ray having a wavelength of 3 to 15 nm is preferably used as the high-energy ray.

A patterning method with such a high-energy ray is preferred.

### Advantageous Effects of Invention

As described above, a resist film with a chemically amplified resist composition containing a polymer containing a repeating unit derived from the sulfonium salt monomer of the present invention, which is represented by the general formula (a), has good solubility in solvents, and is characterized by small acid diffusion because of the large atomic weight of iodine atoms. This can successfully prevent reduction in resolution due to blur caused by acid diffusion, giving improved LWR and CDU. Because of the very large absorption of EUV by iodine atoms at a wavelength of 13.5 nm, secondary electrons are generated from iodine atoms during exposure, resulting in high sensitivity. In addition, the nitro group contained in the cation is a strong electron-withdrawing group, and bonding of this to the aromatic ring of the sulfonium cation reduces the LUMO in the frontier molecular orbital theory to facilitate reception of secondary electrons, promoting the decomposition of the sulfonium cation. In addition, the nitro group, having a resonance structure in which a positive charge is present on the nitrogen atom and a negative charge is present on one oxygen atom, interacts with a proton of a generated acid at the negative charge on the oxygen atom, hence also expected to exert functions as an acid diffusion inhibitory group. The charged state gives high affinity for alkaline developer, and residues after cation decomposition are efficiently removed from alkaline developer, and as a result the risk of post-develop defects in exposed parts is reduced as well. This enables construction of a chemically amplified resist composition having high sensitivity in combination with improved LWR and CDU. Moreover, the aromatic ring acts as a good etching-resistant group, thus being suitable for fine patterning. In particular, a chemically amplified resist composition can be formed that exhibits superior solubility in solvents, is superior in lithographic performance including exposure latitude (EL), LWR, CDU, and depth of focus (DOF) with high sensitivity and high contrast, and is superior also in etching resistance with durability against pattern collapse even in fine patterning in photolithography with use of a high-energy ray such as KrF excimer laser light, ArF excimer laser light, an electron beam (EB), and EUV.

### DESCRIPTION OF EMBODIMENTS

As described above, development of a sulfonium salt monomer to be used as a sulfonium salt quencher, which is a material of a polymer to be contained in a chemically amplified resist composition that exhibits superior solubility in solvents, is superior in lithographic performance with high sensitivity and high contrast, and is superior also in etching resistance with durability against pattern collapse even in fine patterning in photolithography with use of a high-energy ray, a sulfonium salt quencher containing the sulfonium salt monomer, a polymer containing a repeating unit derived from the sulfonium salt quencher, a chemically amplified resist composition containing a base polymer containing the polymer, and a patterning method with the chemically amplified resist composition has been demanded.

The present inventors have diligently examined to achieve the object, and found that a chemically amplified resist composition that exhibits superior solubility in solvents, has high sensitivity, is improved in lithographic performance including EL, LWR, CDU, and DOF, has high contrast and thus high resolution, and is superior also in etching resistance is obtained by using a polymer as a polymer-bound quencher, the polymer containing a repeating unit derived from a sulfonium salt containing a carboxylate anion having a styrene or vinylnaphthalene structure as a polymerizable group and an iodine atom and a sulfonium cation having a nitro group, completing the present invention.

Specifically, the present invention relates to a sulfonium salt monomer represented by the following general formula (a):

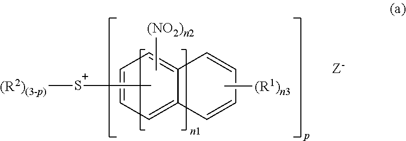

wherein p is 1, 2, or 3, n1 is 0 or 1, n2 is 1 or 2, and n3 is an integer of 0 to 6, provided that 1≤n2+n3≤5 is satisfied if n1 is 0, and 1≤n2+n3≤7 is satisfied if n1 is 1;
R¹ is a halogen atom, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein if n3 is 2 to 6, R¹ groups are identical to or different from each other, and any two R¹ groups are optionally bound together to form a ring together with the carbon atoms to which the two groups are bound;
R² is a halogen atom or a hydrocarbyl group having 1 to 30 carbon atoms and optionally containing a heteroatom, wherein if p is 1, the two R² groups are identical to or different from each other, and any two of the three substituents bound to S⁺ are optionally bound together to form a ring together with the sulfur atom to which the two substituents are bound; and
Z⁻ is a carboxylate anion having an aromatic vinyl structure and an iodine atom.

The following describes the present invention in detail, but the present invention is not limited by the description.

### [Sulfonium Salt Monomer]

The sulfonium salt monomer of the present invention is represented by the following general formula (a):

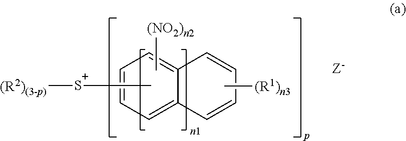

wherein p is 1, 2, or 3, n1 is 0 or 1, n2 is 1 or 2, and n3 is an integer of 0 to 6, provided that 1≤n2+n3≤5 is satisfied if n1 is 0, and 1≤n2+n3≤7 is satisfied if n1 is 1;
R¹ is a halogen atom, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein if n3 is 2 to 6, R¹ groups are identical to or different from each other, and any two R¹ groups are optionally bound together to form a ring together with the carbon atoms to which the two groups are bound;
R² is a halogen atom or a hydrocarbyl group having 1 to 30 carbon atoms and optionally containing a heteroatom, wherein if p is 1, the two R² groups are identical to or different from each other, and any two of the three substituents bound to S⁺ are optionally bound together to form a ring together with the sulfur atom to which the two substituents are bound; and
Z⁻ is a carboxylate anion having an aromatic vinyl structure and an iodine atom.

In the general formula (a), p is 1, 2, or 3.

In the general formula (a), n1 is 0 or 1. While a benzene ring is given if n1 is 0, and a naphthalene ring is given if n1 is 1, the case that n1 is 0 to give a benzene ring is preferred for solubility in solvents. n2 is 1 or 2. n2 is preferably 1 for raw material procurement. n3 is an integer of 0 to 6. n3 is preferably 0, 1, or 2 for raw material procurement. It should be noted that 1≤n2+n3≤5 is satisfied if n1 is 0, and 1≤n2+n3≤7 is satisfied if n1 is 1.

In the general formula (a), R¹ is a halogen atom, a cyano group, a hydroxy group, a carboxy group, an alkoxycarbonyl group, or a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. Examples of the halogen atom include a fluorine atom, a chlorine atom, a bromine atom, and an iodine atom. The hydrocarbyl group and the hydrocarbyl moieties of the hydrocarbyloxy group and hydrocarbylthio group may be each saturated or unsaturated, and may be each linear, branched, or cyclic. Specific examples thereof include an alkyl group having 1 to 20 carbon atoms such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; a cyclic saturated hydrocarbyl group having 3 to 20 carbon atoms such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; an alkenyl group having 2 to 20 carbon atoms such as a vinyl group, a 1-propenyl group, a 2-propenyl group, a butenyl group, and a hexenyl group; a cyclic unsaturated hydrocarbyl group having 3 to 20 carbon atoms such as a cyclohexenyl group; an aryl group having 6 to 20 carbon atoms such as a phenyl group and a naphthyl group; an aralkyl group having 7 to 20 carbon atoms such as a benzyl group, a 1-phenylethyl group, and a 2-phenylethyl group; and a group given by combining any of these. Preferred among them is an aryl group. Some or all of the hydrogen atoms of the hydrocarbyl group may be each replaced with a group containing a heteroatom such as an oxygen atom, a sulfur atom, a nitrogen atom, and a halogen atom, some of the —CH₂-moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained. If n3 is 2 to 6, R¹ groups are identical to or different from each other. If n3 is 2 to 6, any multiple R¹ groups are optionally bound together to form a ring together with the carbon atoms to which the groups are bound. The ring is preferably a 5- to 8-membered ring.

In the general formula (a), R² is a halogen atom or a hydrocarbyl group having 1 to 30 carbon atoms and optionally containing a heteroatom. If p is 1, the two R² groups are identical to or different from each other.

Specific examples of the halogen atom represented by R² include a fluorine atom, a chlorine atom, a bromine atom, and an iodine atom.

The hydrocarbyl group represented by R² may be saturated or unsaturated, and may be linear, branched, or cyclic. Specific examples thereof include an alkyl group having 1 to 30 carbon atoms such as a methyl group, an ethyl group, a n-propyl group, an isopropyl group, a n-butyl group, an isobutyl group, a sec-butyl group, and a tert-butyl group; a cyclic saturated hydrocarbyl group having 3 to 30 carbon atoms such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; an alkenyl group having 2 to 30 carbon atoms such as a vinyl group, a 1-propenyl group, a 2-propenyl group, a butenyl group, and a hexenyl group; a cyclic unsaturated hydrocarbyl group having 3 to 30 carbon atoms such as a cyclohexenyl group; an aryl group having 6 to 30 carbon atoms such as a phenyl group, a naphthyl group, and a thienyl group; an aralkyl group having 7 to 30 carbon atoms such as a benzyl group, a 1-phenylethyl group, and a 2-phenylethyl group; and a group given by combining any of these, and an aryl group is preferred. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a nitro group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

Any two of the three substituents bound to S⁺ are optionally bound together to form a ring together with the sulfur atom to which the two substituents are bound. Specific examples of the structure of the ring in this case include those represented by the following formulae:

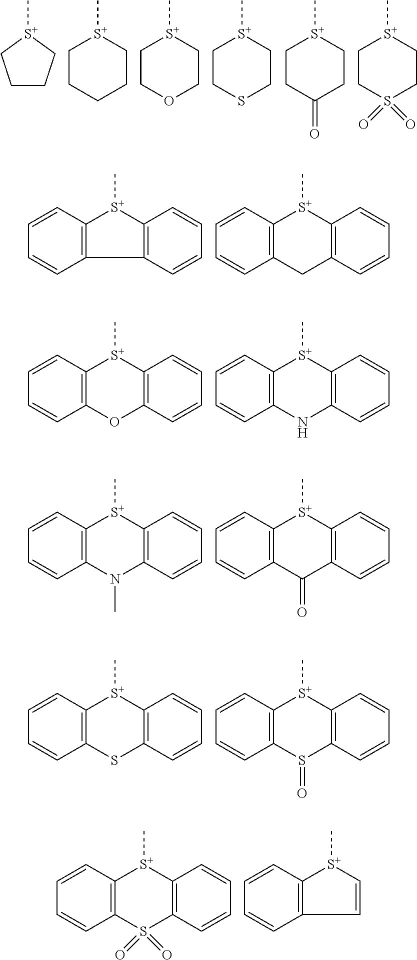

wherein each dashed line indicates bonding.

The sulfonium salt monomer represented by the general formula (a) is preferably one represented by the following general formula (a1):

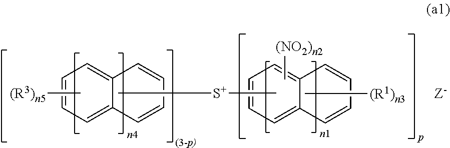

wherein p, n1 to n3, R¹, and Z⁻ are as specified above;
n4 is 0 or 1, and n5 is an integer of 0 to 5; and
R³ is a halogen atom, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein if n5 is 2 to 5, R³ groups are identical to or different from each other, and any two R³ groups are optionally bound together to form a ring together with the carbon atoms to which the two groups are bound.

In the general formula (a1), n4 is 0 or 1. While a benzene ring is given if n4 is 0, and a naphthalene ring is given if n4 is 1, the case that n4 is 0 to give a benzene ring is preferred for solubility in solvents. n5 is an integer of 0 to 5. n5 is preferably 0, 1, or 2 for raw material procurement.

In the general formula (a1), R³ is a halogen atom, a cyano group, a hydroxy group, a carboxy group, an alkoxycarbonyl group, or a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. The hydrocarbyl group and the hydrocarbyl moieties of the hydrocarbyloxy group and hydrocarbylthio group may be each saturated or unsaturated, and may be each linear, branched, or cyclic. Specific examples thereof include, but are not limited to, those shown as examples of the hydrocarbyl group represented by R¹. If n5 is 2 to 5, R³ groups are identical to or different from each other, and any two R³ groups are optionally bound together to form a ring together with the carbon atoms to which the groups are bound. The maximum value of the number of carbon atoms of groups bound to S⁺ including R³ groups is 30.

Examples of the cation of the sulfonium salt monomer represented by the general formula (a) include, but are not limited to, those shown in the following:

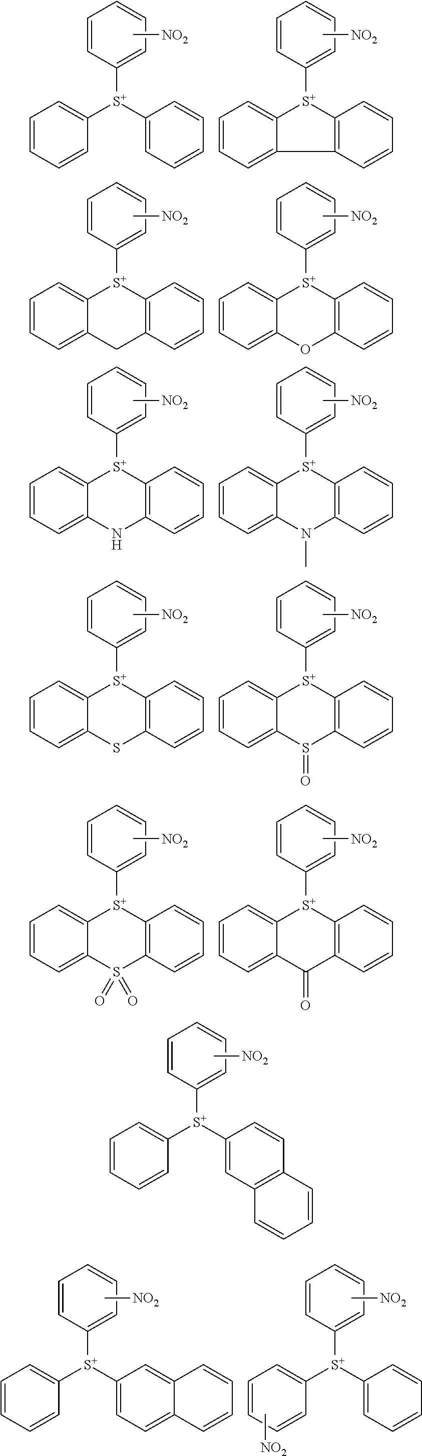

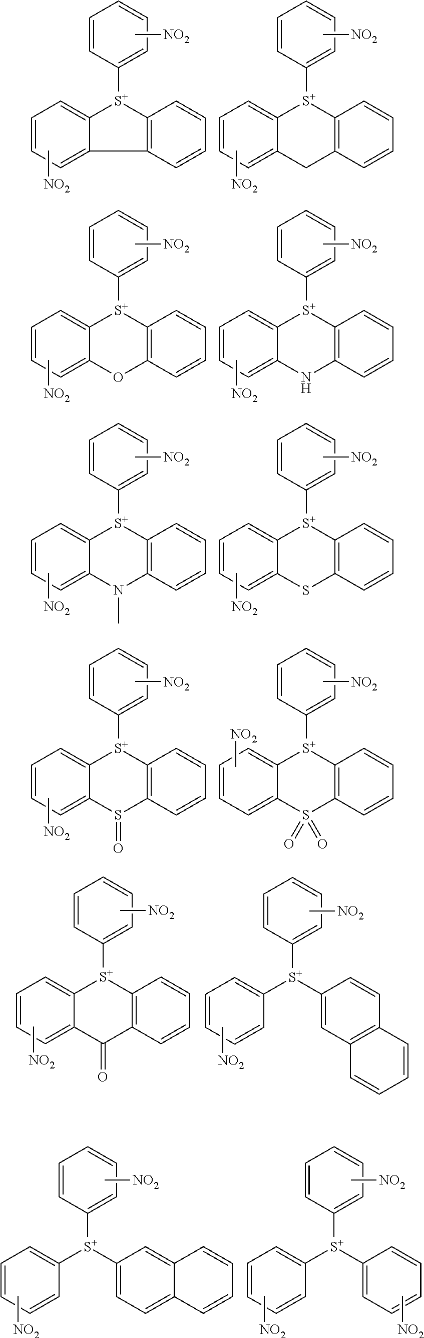

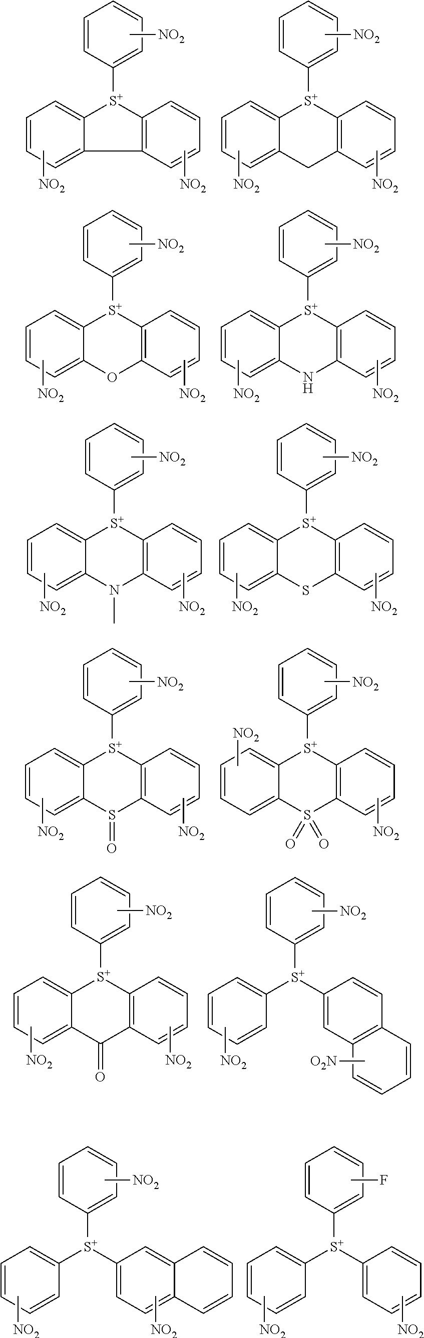

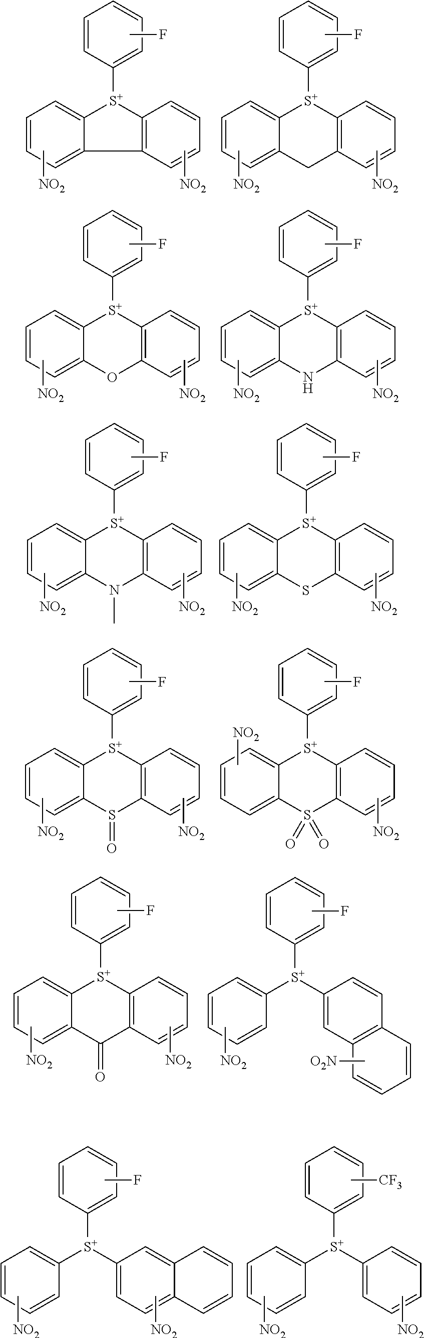

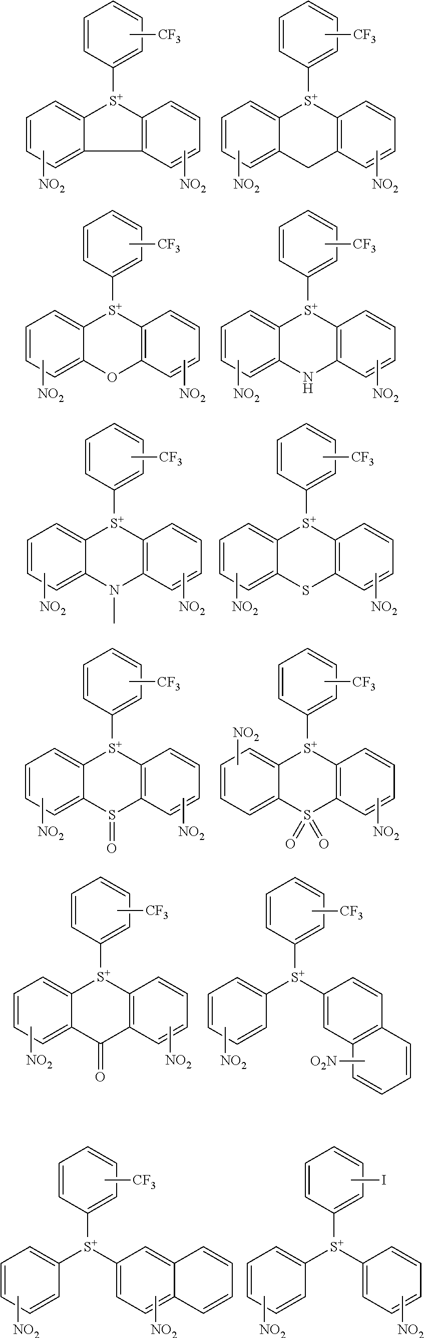

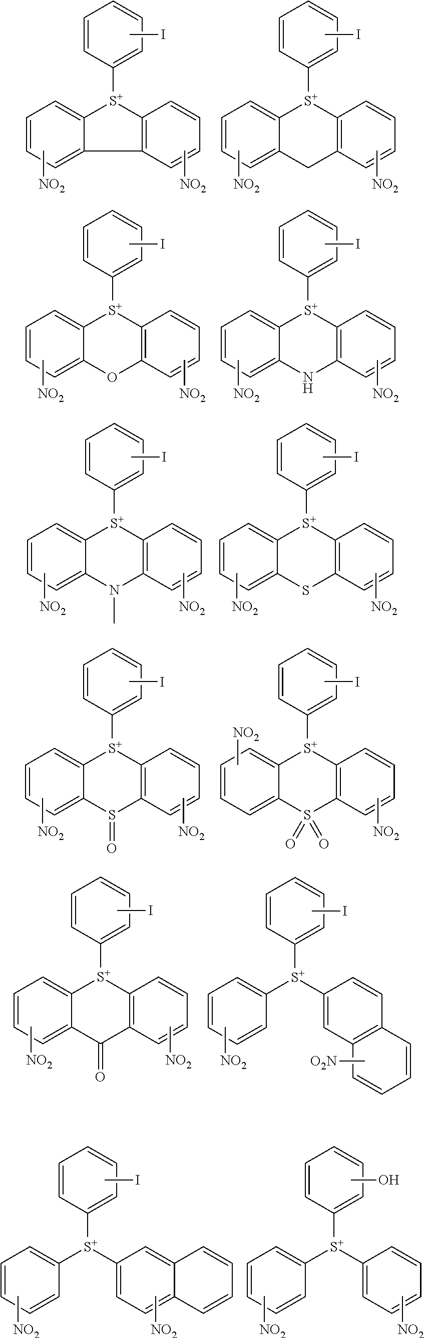

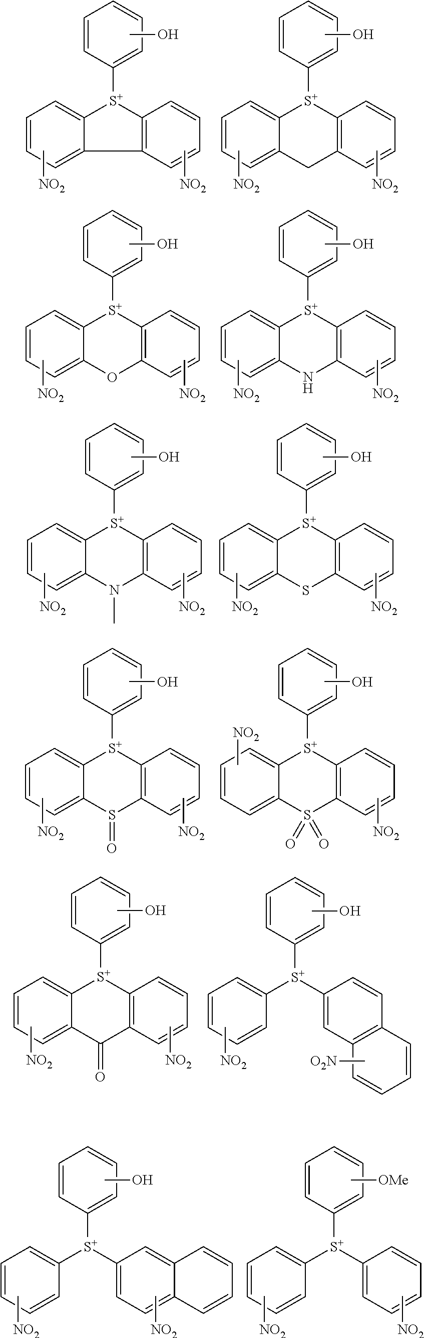

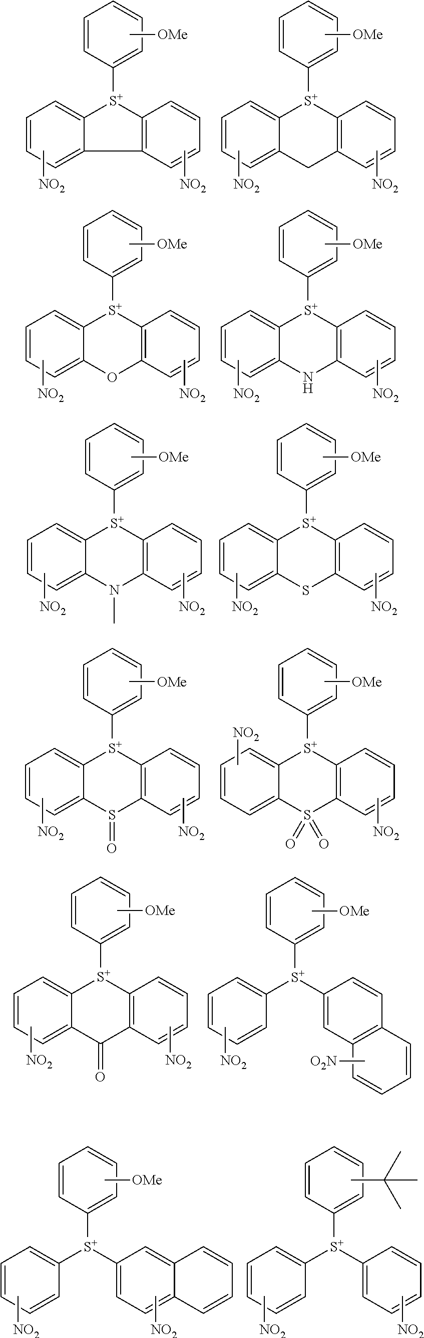

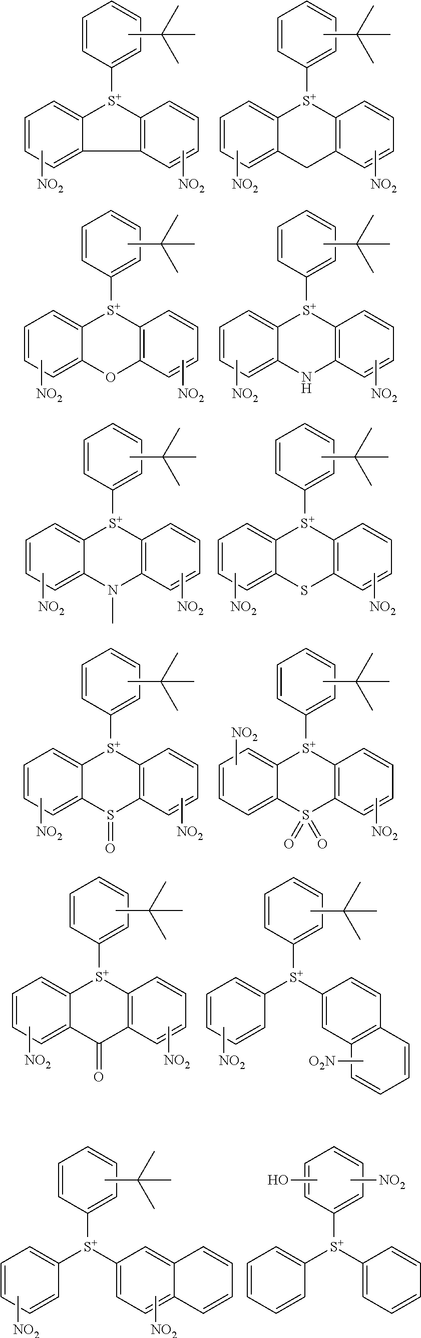

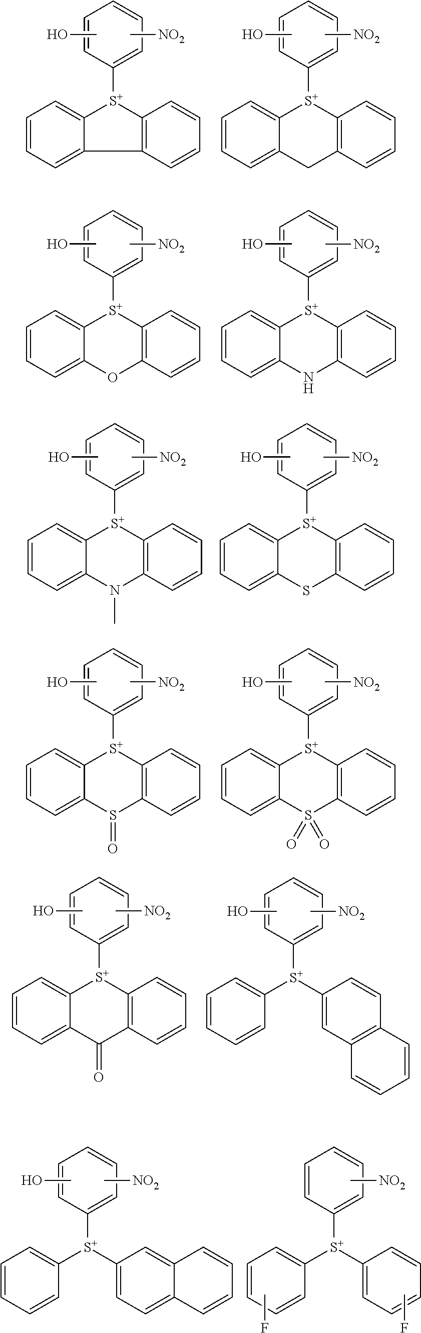

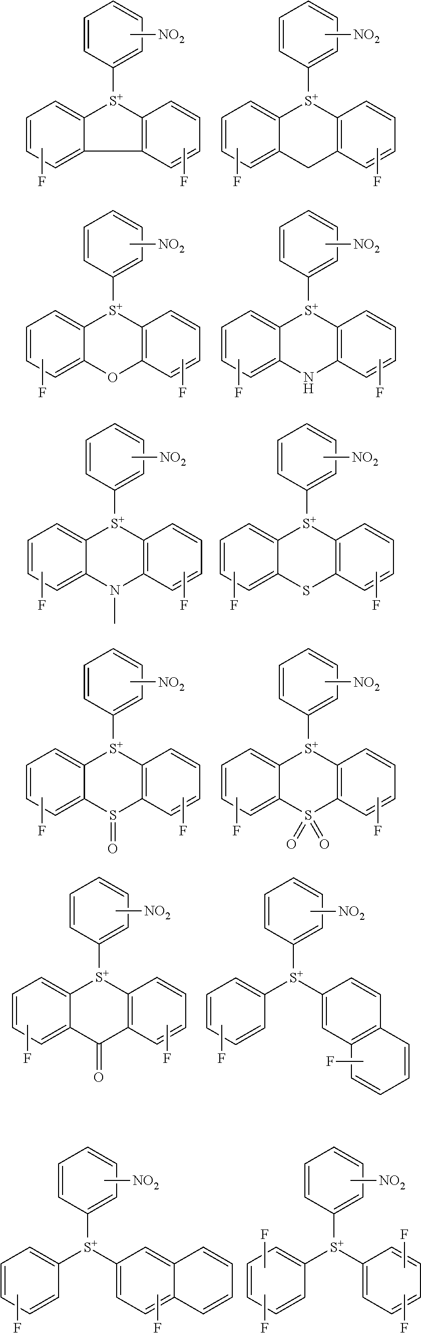

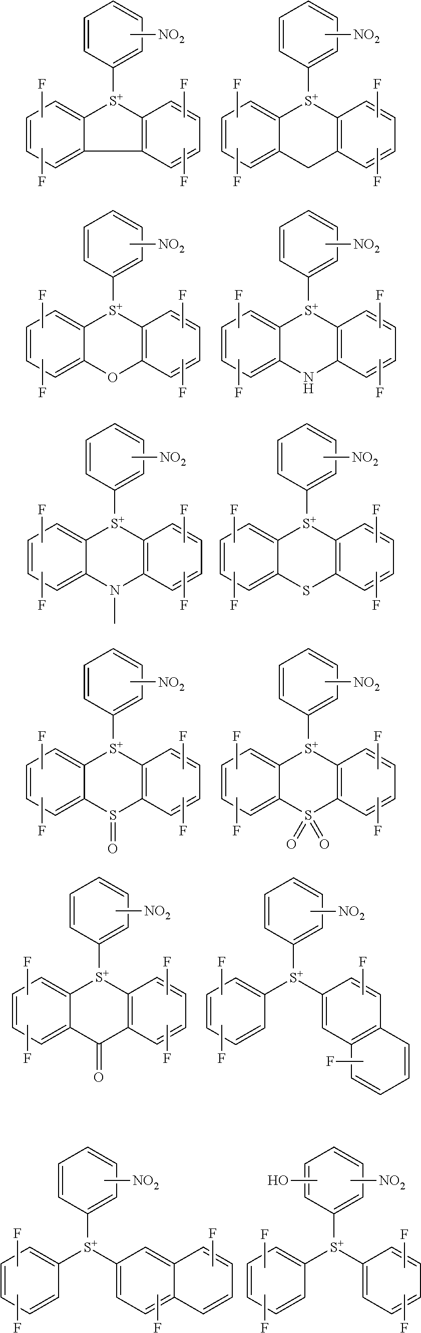

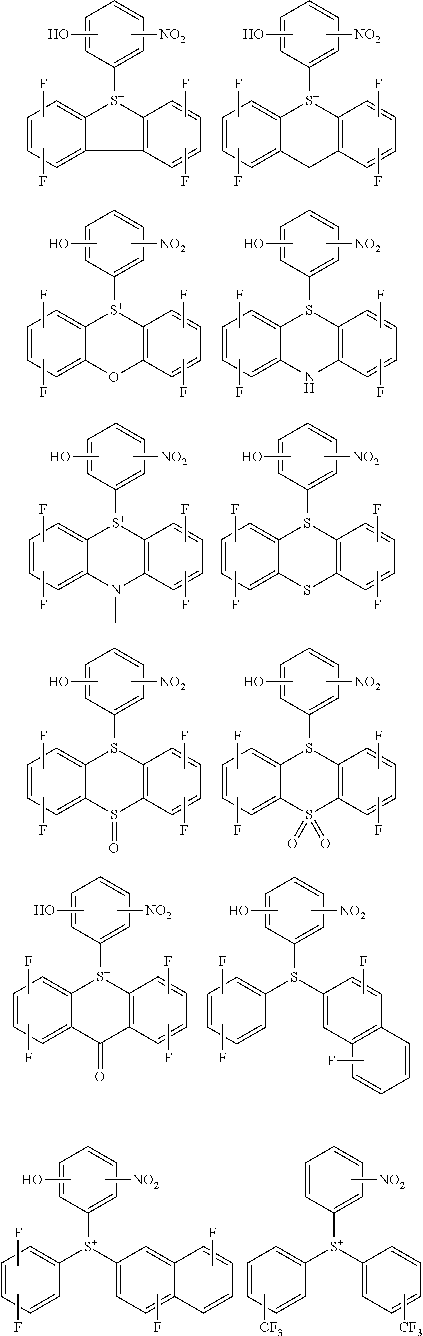

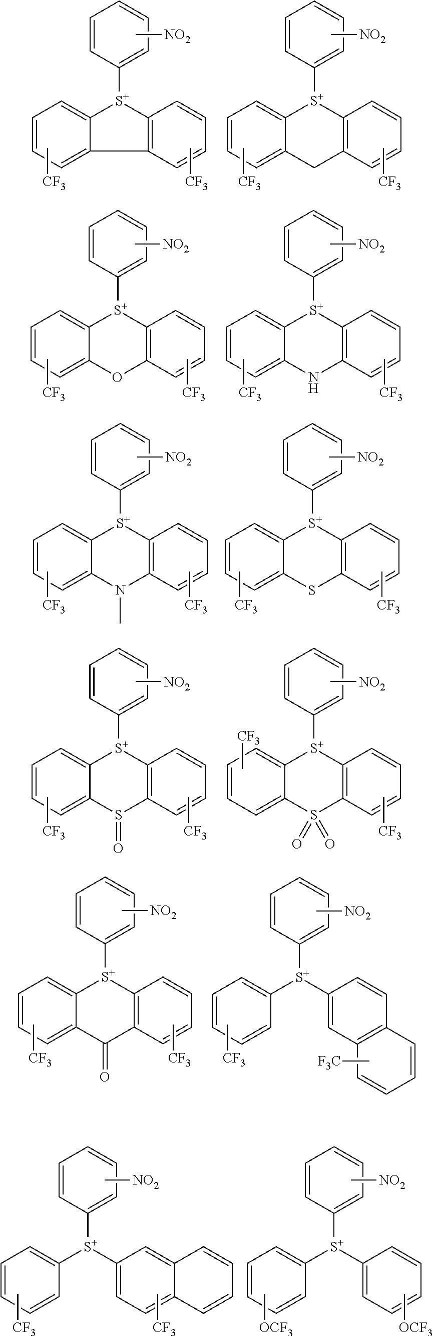

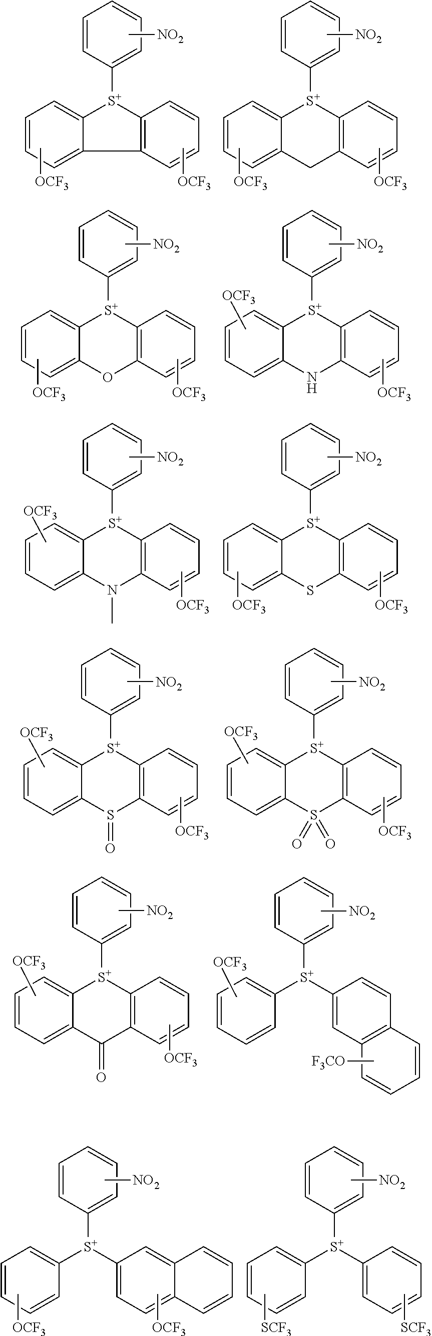

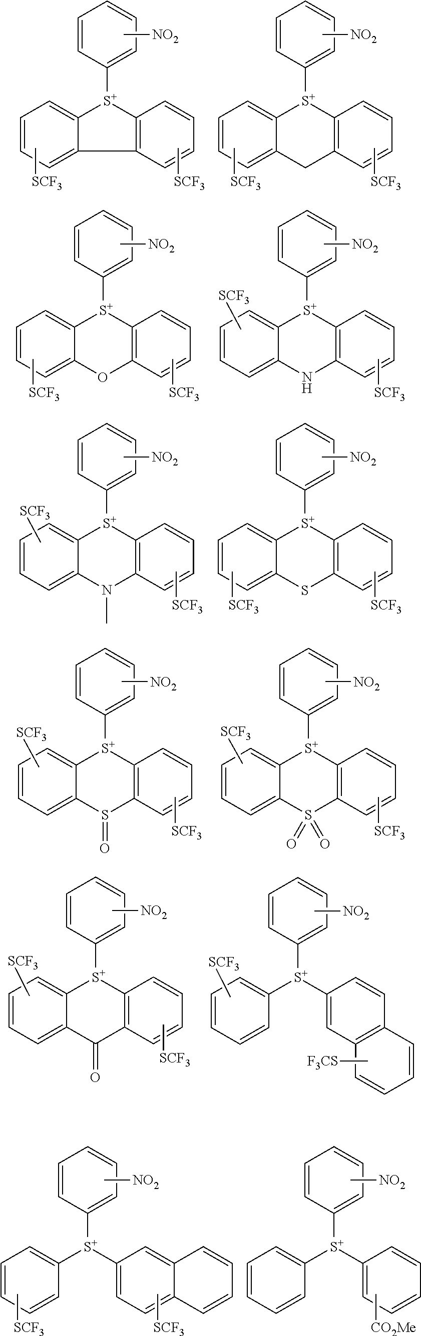

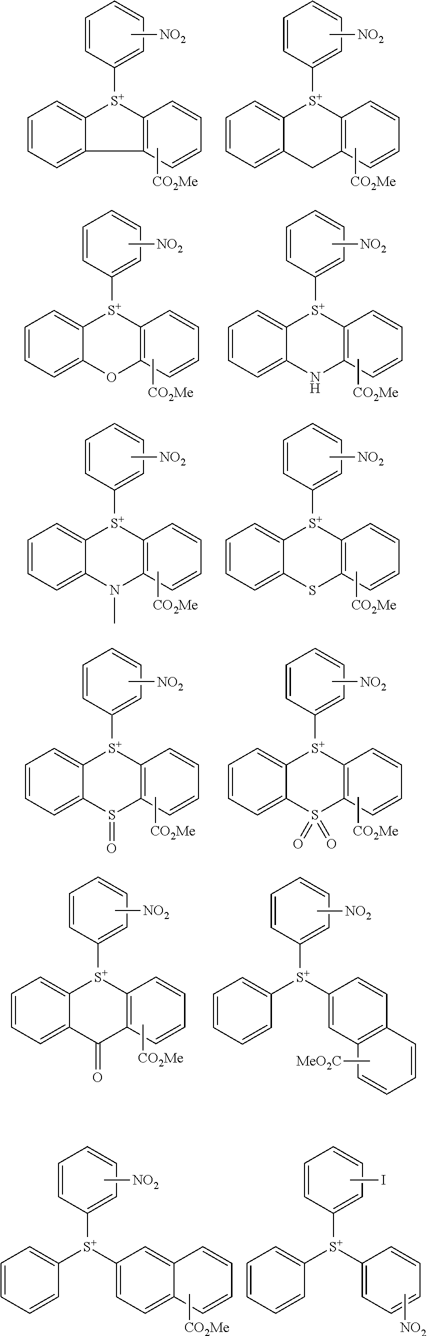

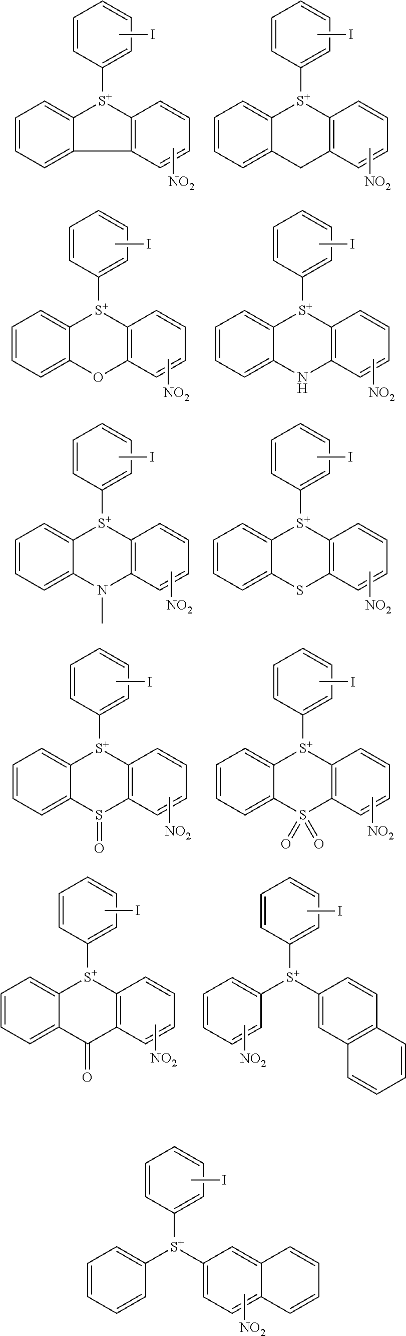

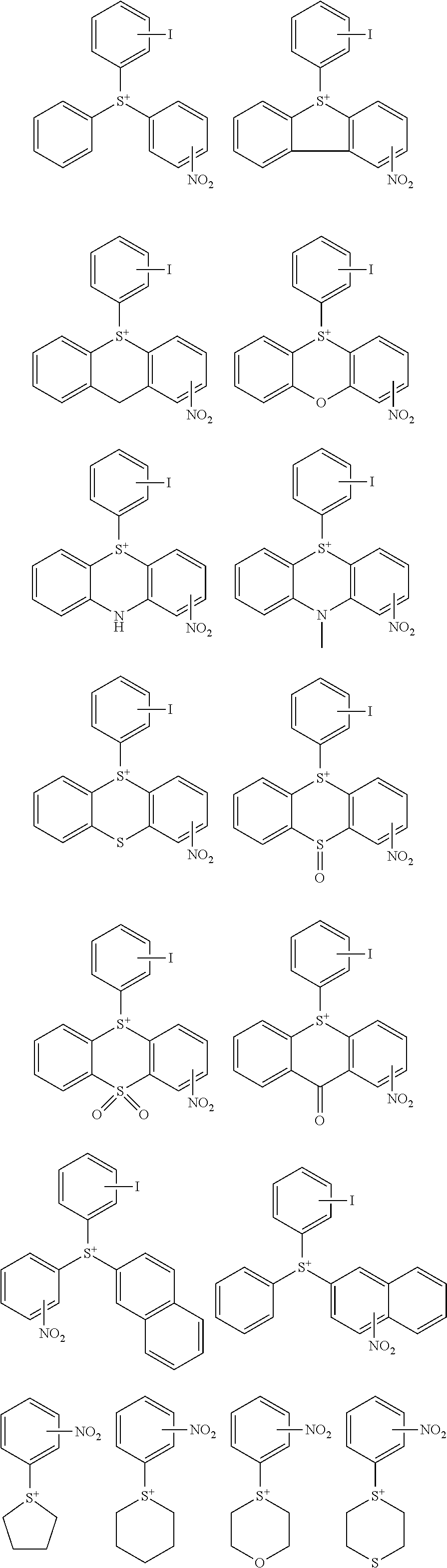

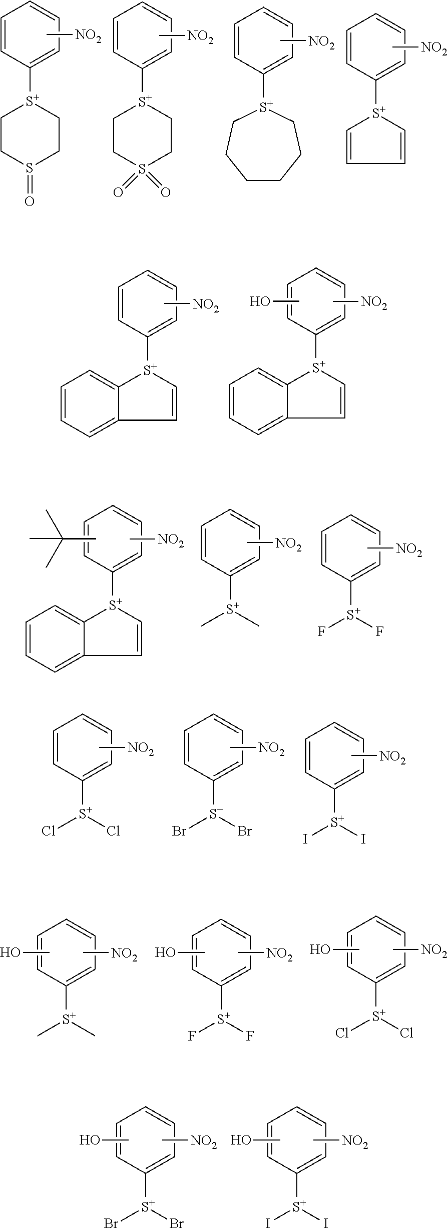

In the general formulae (a) and (a1), Z⁻ is a carboxylate anion having an aromatic vinyl structure and an iodine atom. The Z⁻ is preferably represented by the following general formula (Z):

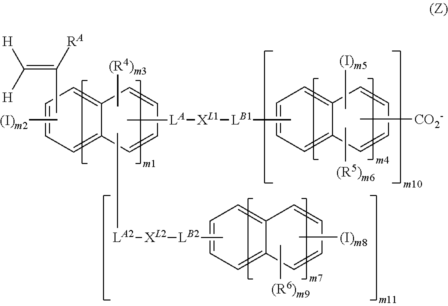

wherein m1 is 0 or 1, m2 is an integer of 0 to 4, m3 is an integer of 0 to 3, m4 is 0 or 1, m5 is an integer of 0 to 4, m6 is an integer of 0 to 3, m7 is 0 or 1, m8 is an integer of 0 to 4, m9 is an integer of 0 to 3, m10 is 0 or 1, and m11 is 0 or 1, provided that 0≤m2+m3+m11≤4 is satisfied if m1 is 0, and 0≤m2+m3+m11≤6 is satisfied if m1 is 1, that 0≤m5+m6≤4 is satisfied if m4 is 0, and 0≤m5+m6≤6 is satisfied if m4 is 1, that 0≤m8+m9≤5 is satisfied if m7 is 0, and 0≤m8+m9≤7 is satisfied if m7 is 1, and that 1≤m2+m5+m8≤4 is satisfied;
R^{A} is a hydrogen atom, a fluorine atom, a methyl group, or a trifluoromethyl group;
R⁴, R⁵, and R⁶ are each a halogen atom being not an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein: if m3 is 2 or 3, R⁴ groups are identical to or different from each other, and any two R⁴ groups are optionally bound together to form a ring together with the carbon atoms to which the two groups are bound; if m6 is 2 or 3, R⁵ groups are identical to or different from each other, and any two R⁵ groups are optionally bound together to form a ring together with the carbon atoms to which the two groups are bound; and if m9 is 2 or 3, R⁶ groups are identical to or different from each other, and any two R⁶ groups are optionally bound together to form a ring together with the carbon atoms to which the two groups are bound;
L^{A1}, L^{A2}, L^{B1}, and L^{B2} are each independently a single bond, an ether bond, a carbonyl group, an ester bond, a sulfonate bond, an amide bond, a sulfonamide bond, a carbonate bond, or a carbamate bond; and X^{L1} and X^{L2} are each independently a single bond or a hydrocarbylene group having 1 to 40 carbon atoms and optionally containing a heteroatom.

In the general formula (Z), m1 is 0 or 1. While a benzene ring is given if m1 is 0, and a naphthalene ring is given if m1 is 1, the case that m1 is 0 to give a benzene ring is preferred for solubility in solvent. m2 is an integer of 0 to 4. m2 is preferably 0, 1, 2, or 3, more preferably 0, 1, or 2, and further preferably 0 or 1 for raw material procurement. m3 is an integer of 0 to 3.

In the general formula (Z), m4 is 0 or 1. While a benzene ring is given if m4 is 0, and a naphthalene ring is given if m4 is 1, the case that m4 is 0 to give a benzene ring is preferred for solubility in solvents. m5 is an integer of 0 to 4. m5 is preferably 0, 1, 2, or 3, and more preferably 0, 1, or 2 for raw material procurement. m6 is an integer of 0 to 3.

In the general formula (Z), m7 is 0 or 1. While a benzene ring is given if m7 is 0, and a naphthalene ring is given if m7 is 1, the case that m7 is 0 to give a benzene ring is preferred for solubility in solvents. m8 is an integer of 1 to 4. m8 is preferably 1, 2, or 3, and more preferably 1 or 2 for raw material procurement. m9 is an integer of 0 to 3.

In the general formula (Z), m10 is 0 or 1. m11 is 0 or 1.

It should be noted: that 0≤m2+m3+m11≤4 is satisfied if m1 is 0, and 0≤m2+m3+m11≤6 is satisfied if m1 is 1; that 0≤m5+m6≤4 is satisfied if m4 is 0, and 0≤m5+m6≤6 is satisfied if m4 is 1; and that 0≤m8+m9≤5 is satisfied if m7 is 0, and 0≤m8+m9≤7 is satisfied if m7 is 1. For the number of iodine atoms in the anion, more iodine atoms result in enhanced absorption particularly to EUV, but in poor solubility in solvents, which may disadvantageously cause precipitation in the resist composition, and for this reason 1≤m2+m5+m8≤4 is preferably satisfied.

In the general formula (Z), R^{A} is a hydrogen atom, a fluorine atom, a methyl group, or a trifluoromethyl group. Among them, R^{A} is preferably a hydrogen atom or a methyl group, and more preferably a hydrogen atom.

In the general formula (Z), R⁴ is a halogen atom being not an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. The halogen atom being not an iodine atom is preferably a fluorine atom, a chlorine atom, or a bromine atom, and more preferably a fluorine atom. The hydrocarbyl group and the hydrocarbyl moieties of the hydrocarbyloxy group and hydrocarbylthio group may be each saturated or unsaturated, and may be each linear, branched, or cyclic. Specific examples thereof include an alkyl group having 1 to 20 carbon atoms such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; a cyclic saturated hydrocarbyl group having 3 to 20 carbon atoms such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; an alkenyl group having 2 to 20 carbon atoms such as a vinyl group, an allyl group, a propenyl group, a butenyl group, and a hexenyl group; a cyclic unsaturated hydrocarbyl group having 3 to 20 carbon atoms such as a cyclohexenyl group; an aryl group having 6 to 20 carbon atoms such as a phenyl group and a naphthyl group; an aralkyl group having 7 to 20 carbon atoms such as a benzyl group, a 1-phenylethyl group, and a 2-phenylethyl group; and a group given by combining any of these. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained. If m3 is 2 or 3, R⁴ groups are identical to or different from each other.

If m3 is 2 or 3, any two R⁴ groups are optionally bound together to form a ring together with the carbon atoms of the aromatic ring or aromatic rings to which the two groups are bound. Specific examples of the ring formed in this case include a cyclopropane ring, a cyclobutane ring, a cyclopentane ring, a cyclohexane ring, a norbornane ring, and an adamantane ring. Some or all of the hydrogen atoms in the ring may be each replaced with a group containing a heteroatom such as an oxygen atom, a sulfur atom, a nitrogen atom, and a halogen atom, some of the —CH₂— moieties in the ring may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

In the general formula (Z), R⁵ is a halogen atom being not an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. Specific examples of the halogen atom being not an iodine atom include a fluorine atom, a chlorine atom, and a bromine atom. The hydrocarbyl group and the hydrocarbyl moieties of the hydrocarbyloxy group and hydrocarbylthio group may be each saturated or unsaturated, and may be each linear, branched, or cyclic. Specific examples thereof include, but are not limited to, those shown as examples of the hydrocarbyl group represented by R⁴. If m6 is 2 or 3, R⁵ groups are identical to or different from each other.

If m6 is 2 or 3, any two R⁵ groups are optionally bound together to form a ring together with the carbon atoms to which the two groups are bound. The ring is preferably a 5- to 8-membered ring.

In the general formula (Z), R⁶ is a halogen atom being not an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. Specific examples of the halogen atom being not an iodine atom include a fluorine atom, a chlorine atom, and a bromine atom. The hydrocarbyl group and the hydrocarbyl moieties of the hydrocarbyloxy group and hydrocarbylthio group may be each saturated or unsaturated, and may be each linear, branched, or cyclic. Specific examples thereof include, but are not limited to, those shown as examples of the hydrocarbyl group represented by R⁴. If m9 is 2 or 3, R⁶ groups are identical to or different from each other.

If m9 is 2 or 3, any two R⁶ groups are optionally bound together to form a ring together with the carbon atoms to which the two groups are bound. The ring is preferably a 5- to 8-membered ring.

In the general formula (Z), L^{A1}, L^{A2}, L^{B1}, and L^{B2} are each independently a single bond, an ether bond, a carbonyl group, an ester bond, a sulfonate bond, an amide bond, a sulfonamide bond, a carbonate bond, or a carbamate bond. Among them, L^{A1} is preferably a single bond, an ether bond, an ester bond, or a sulfonate bond, and more preferably an ether bond, an ester bond, or a sulfonate bond. L^{A2} is preferably a single bond, an ether bond, an ester bond, an amide bond, a sulfonamide bond, or a sulfonate bond, and more preferably an ester bond or a sulfonate bond. L^{B1} is preferably a single bond, an ether bond, an ester bond, an amide bond, or a sulfonate bond, and more preferably a single bond, an ether bond, or an ester bond. L^{B2} is preferably a single bond, an ether bond, an ester bond, an amide bond, or a sulfonate bond, and more preferably a single bond, an ether bond, or an ester bond.

If m11 is 1, L^{A1} and L^{A2} are preferably bound to adjacent carbon atoms in the aromatic ring or aromatic rings. In this case, the substituent containing the fluorosulfonate anion structure and the substituent containing an aromatic ring substituted with an iodine atom are present at positions spatially close to each other, and hence higher sensitivity is expected to be achieved.

In the general formula (Z), X^{L1} and X^{L2} are each independently a single bond or a hydrocarbylene group having 1 to 40 carbon atoms and optionally containing a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom.

Specific examples of the hydrocarbylene group represented by X^{L1} and X^{L2}, having 1 to 40 carbon atoms and optionally containing a heteroatom, include, but are not limited to, those shown below. In each of the following formulae, the symbols * indicate bonding to L^{A1} and L^{B1}, or to L^{A2} and L^{B2}.

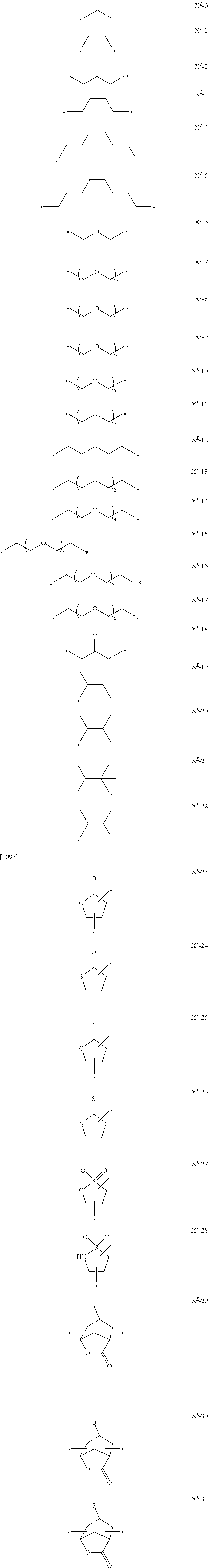

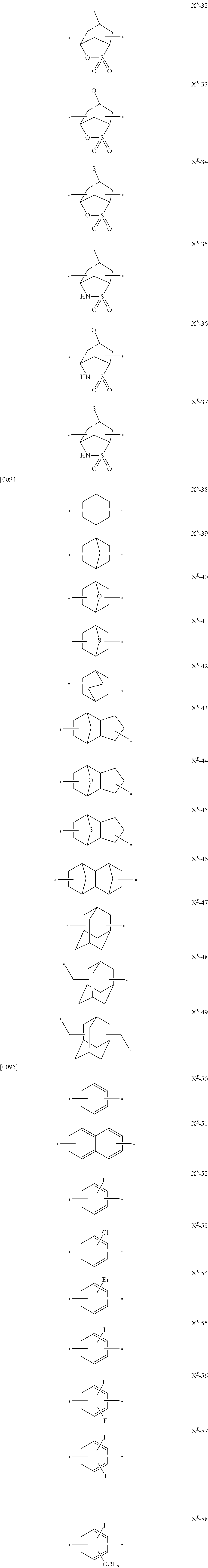

Among them, X^{L}-0 to X^{L}-22, X^{L}-29 to X^{L}-34, and X^{L}-47 to X^{L}-58 are preferred.

The anion of the onium salt monomer represented by the general formula (a) preferably contains no salicylate structure. Specific examples of this anion include, but are not limited to, those shown below. In the following formulae, R^{A} is as specified above, and Me is a methyl group. The bonding positions of substituents in each aromatic ring may be interchanged therein.

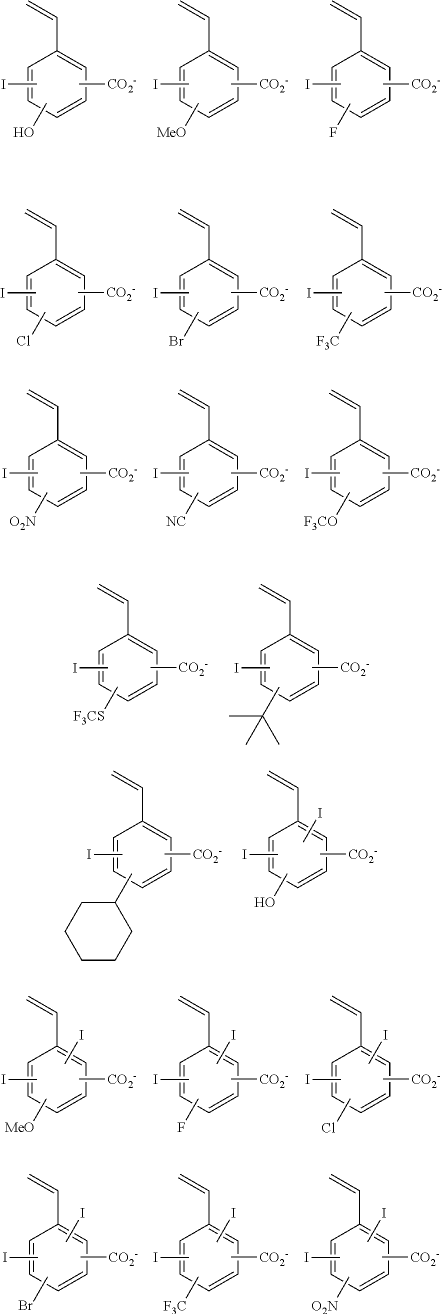

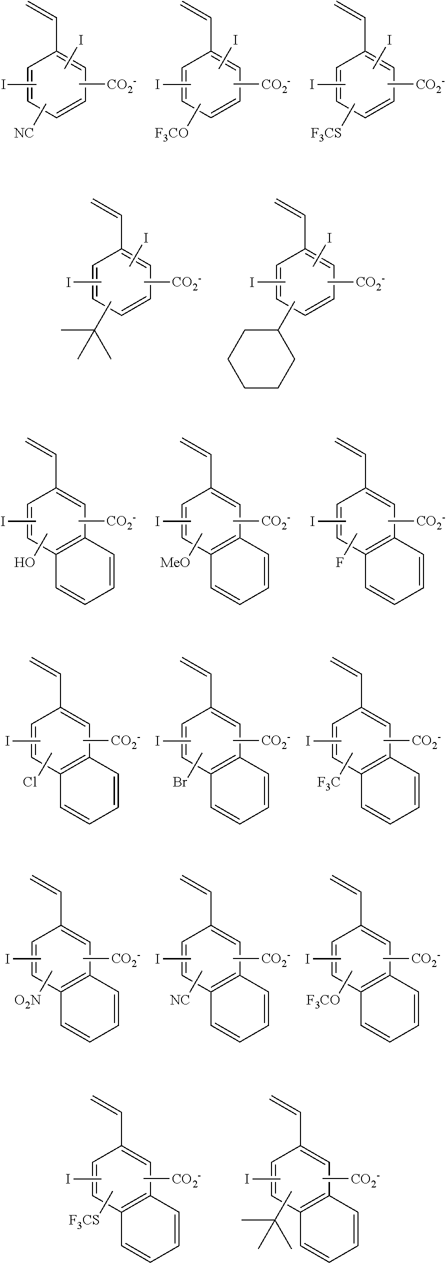

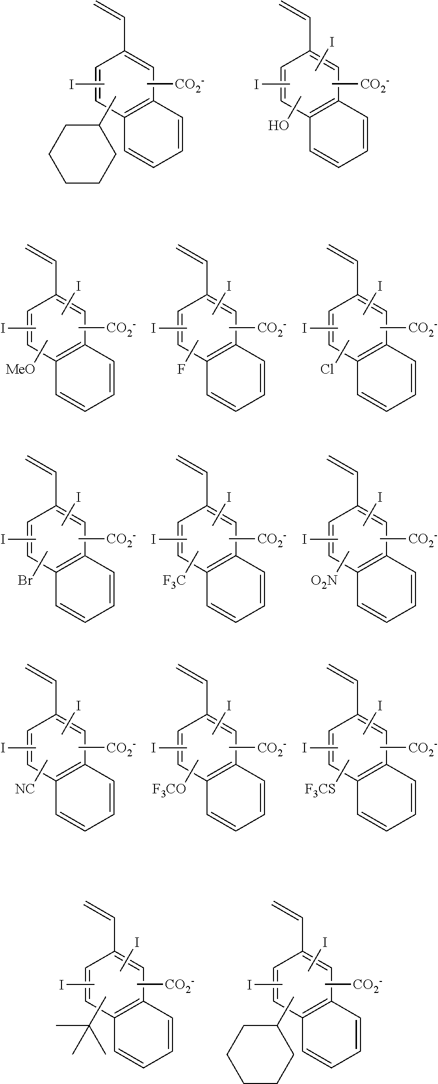

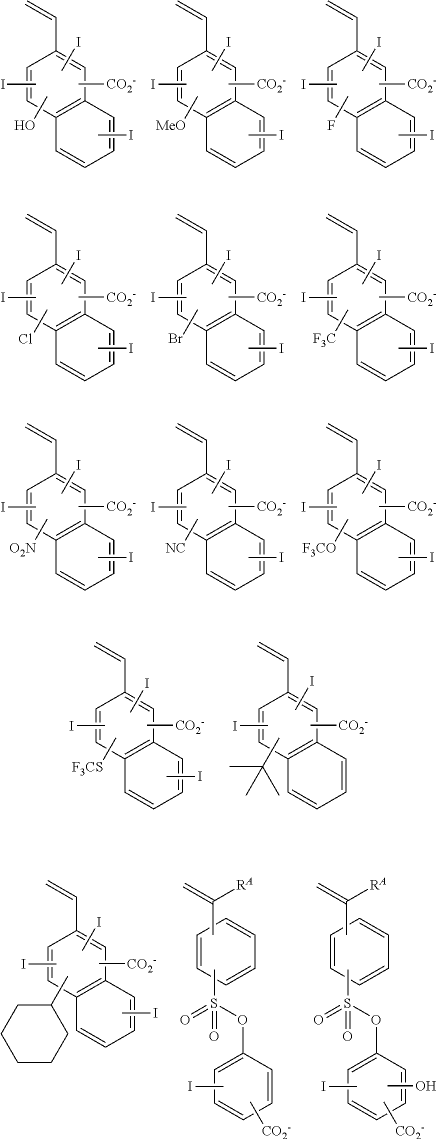

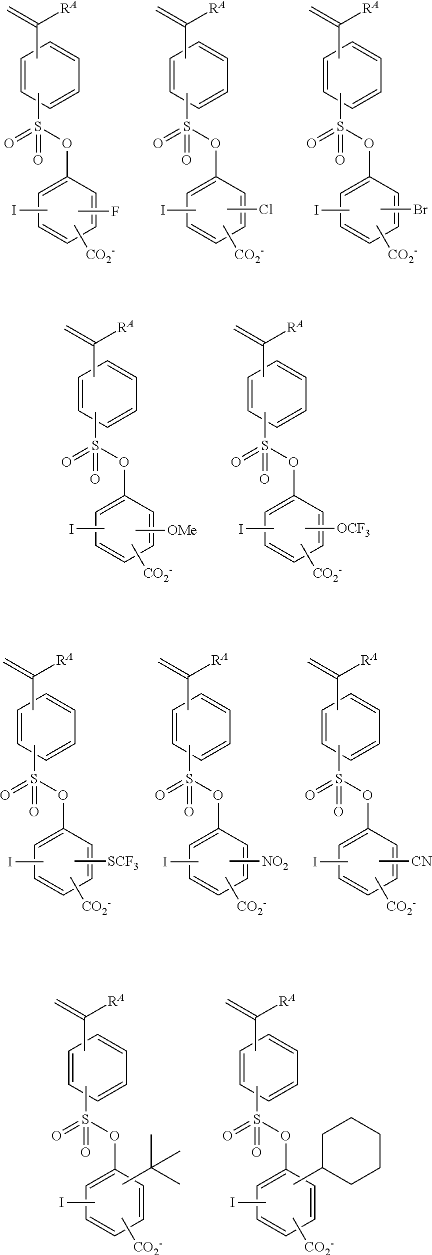

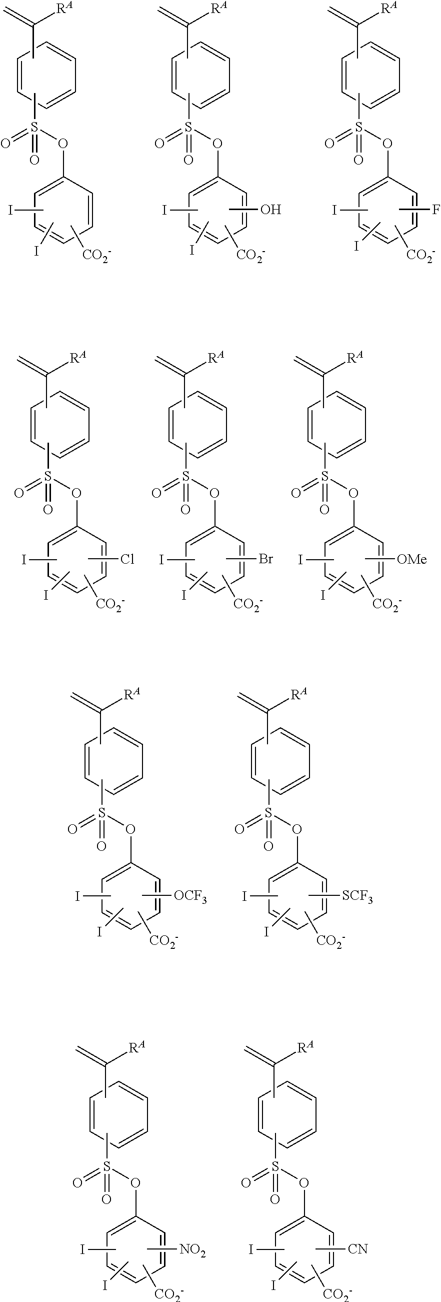

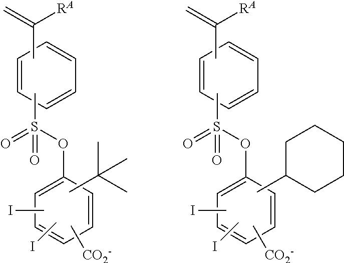

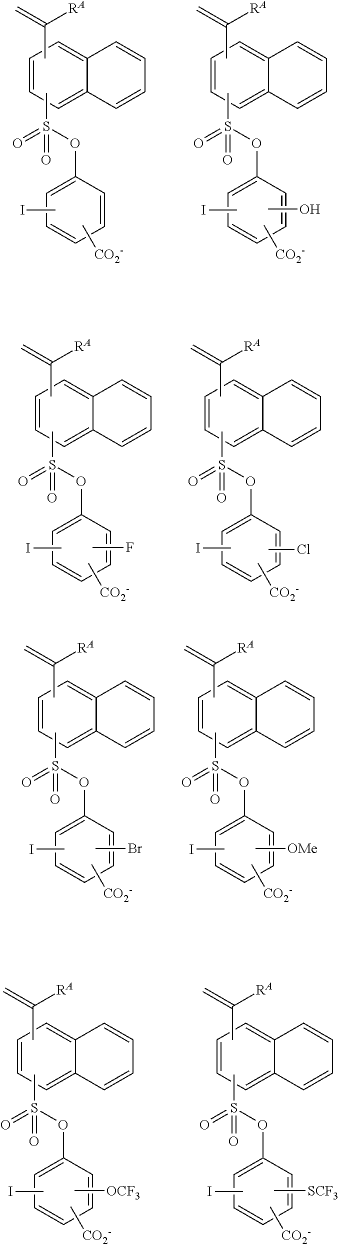

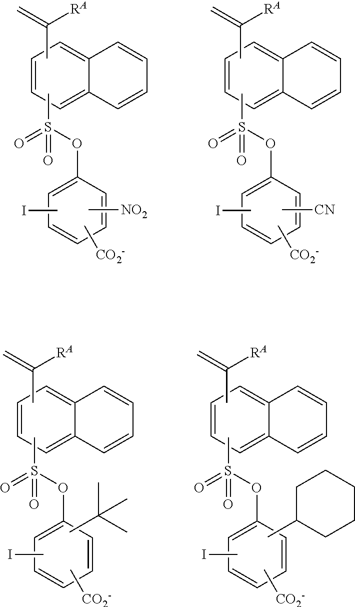

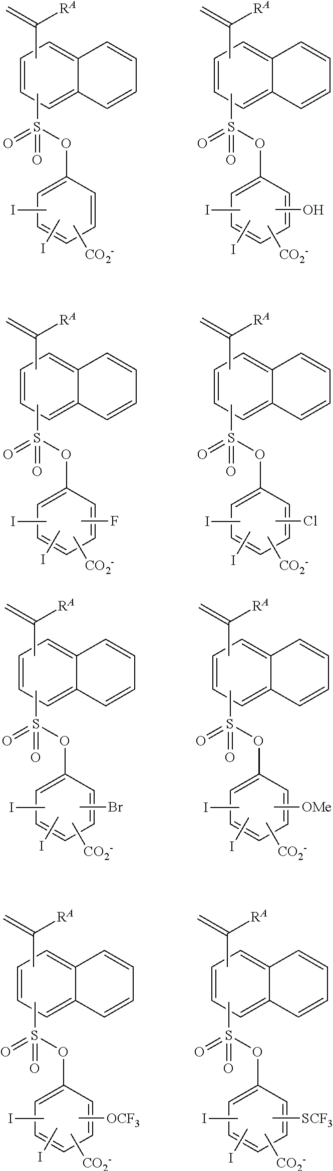

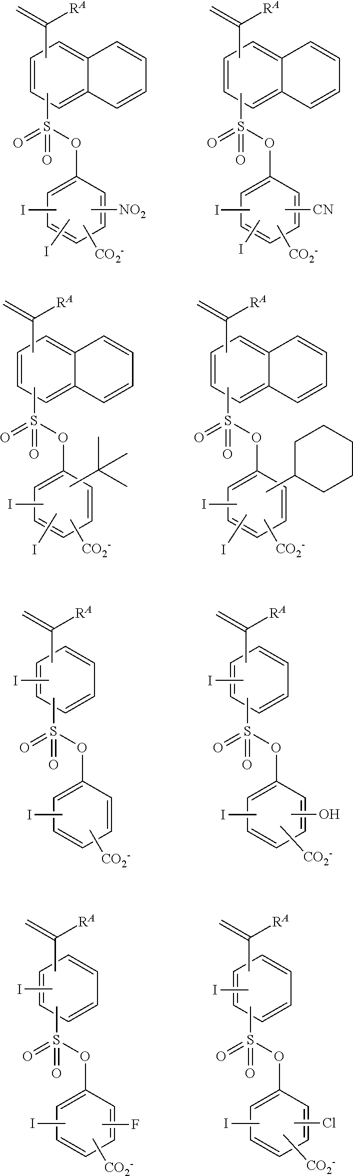

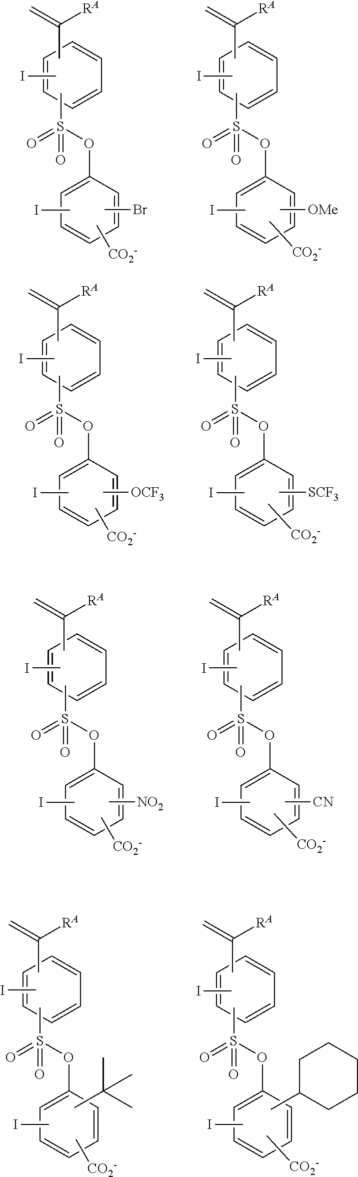

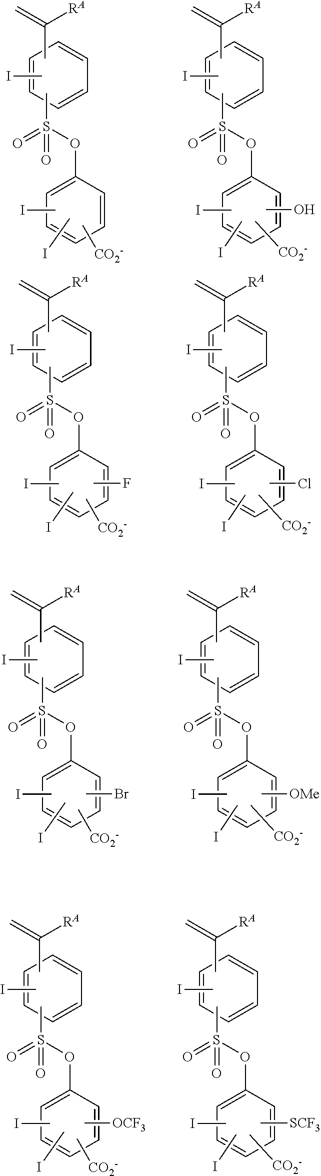

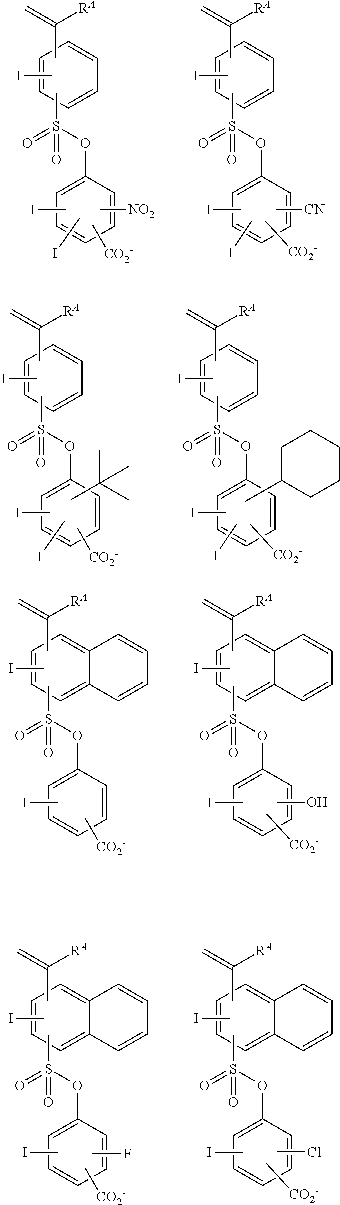

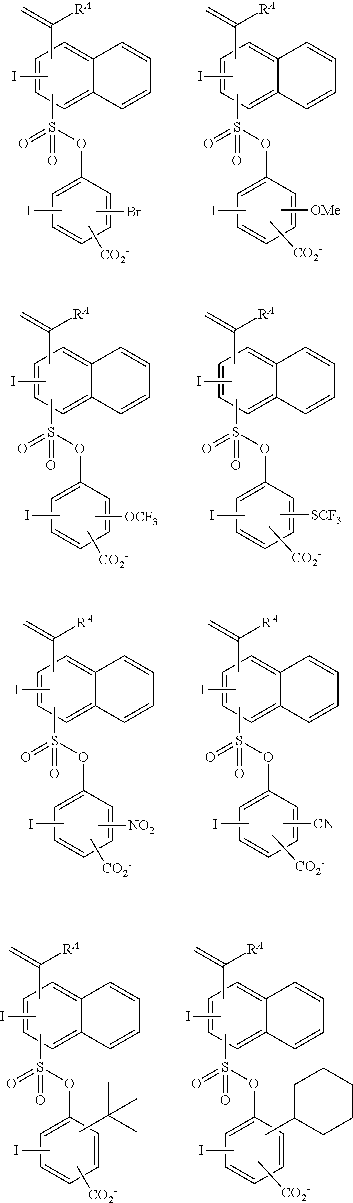

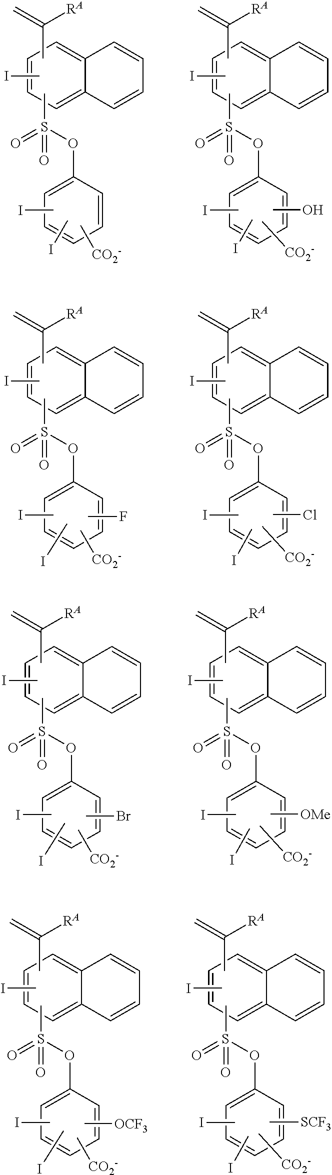

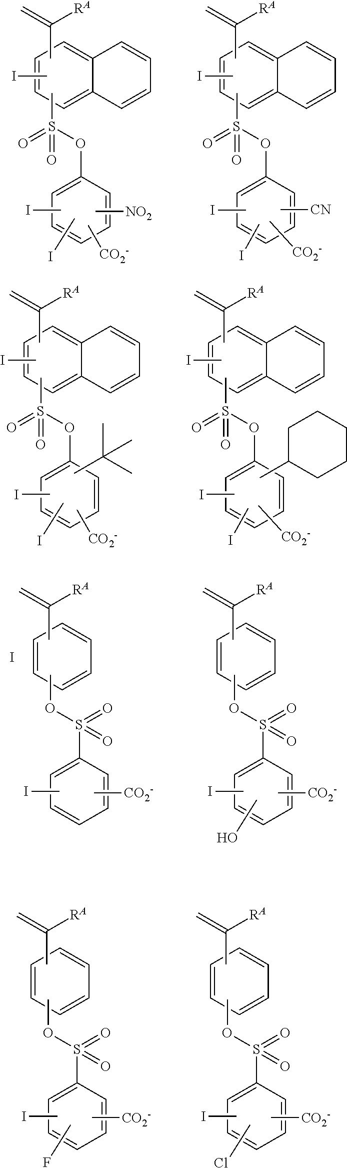

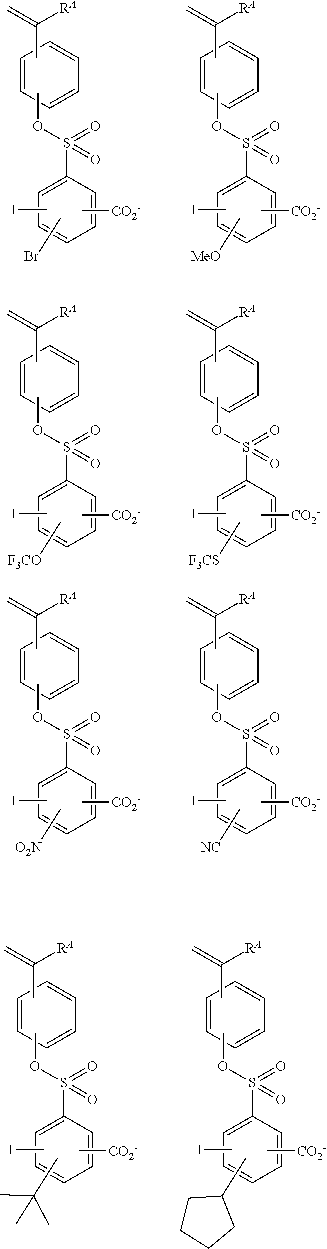

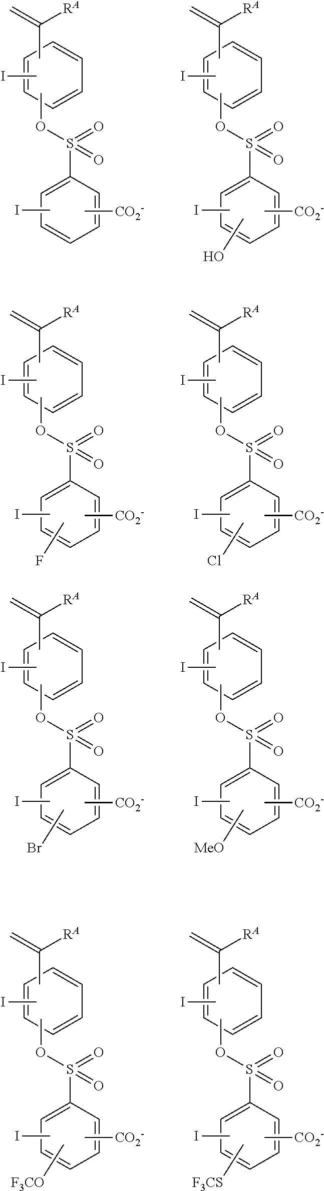

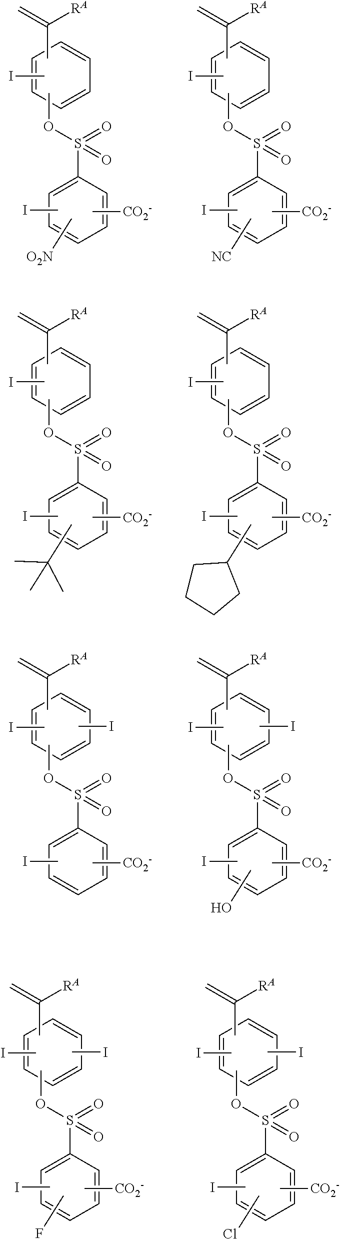

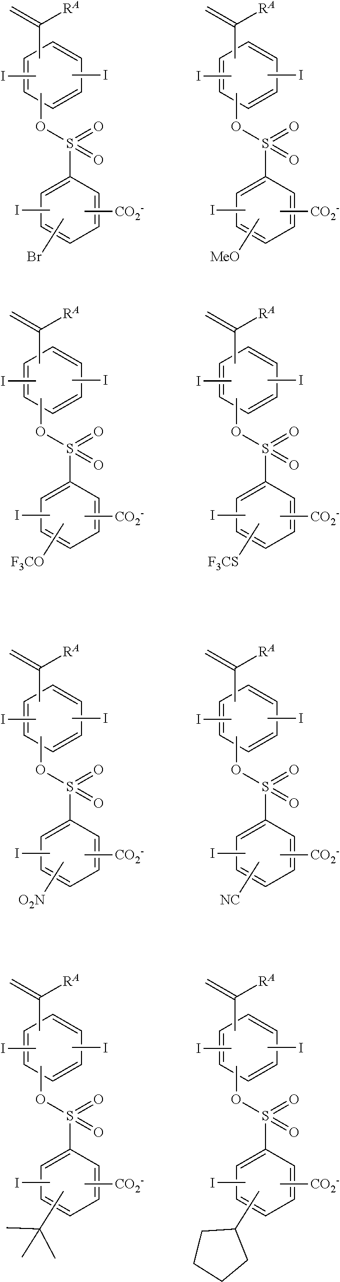

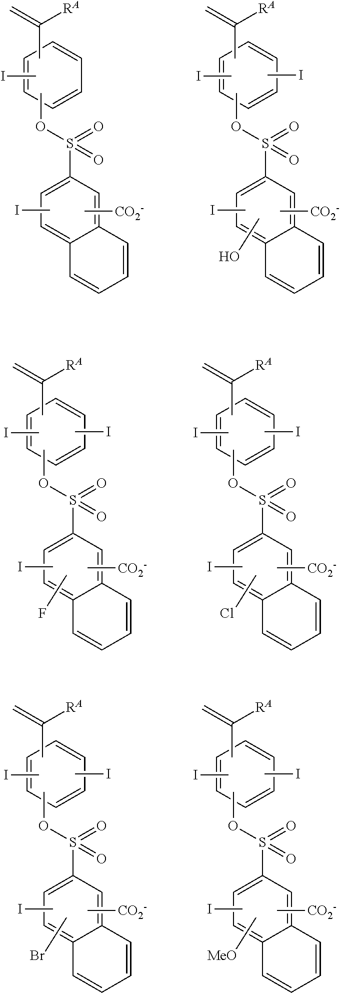

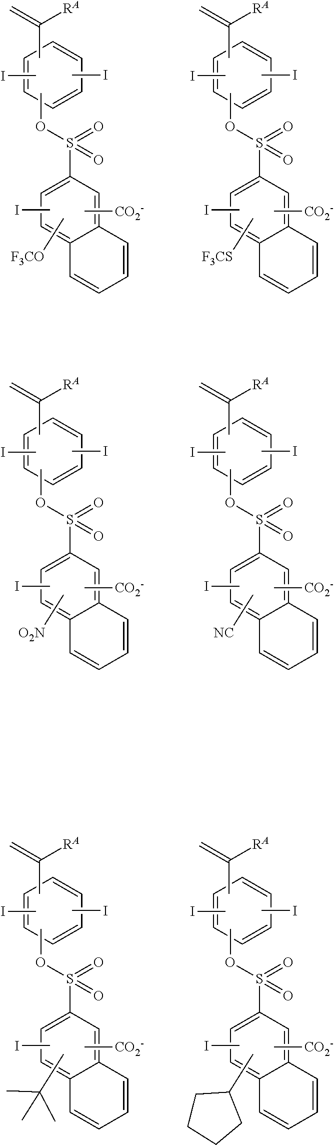

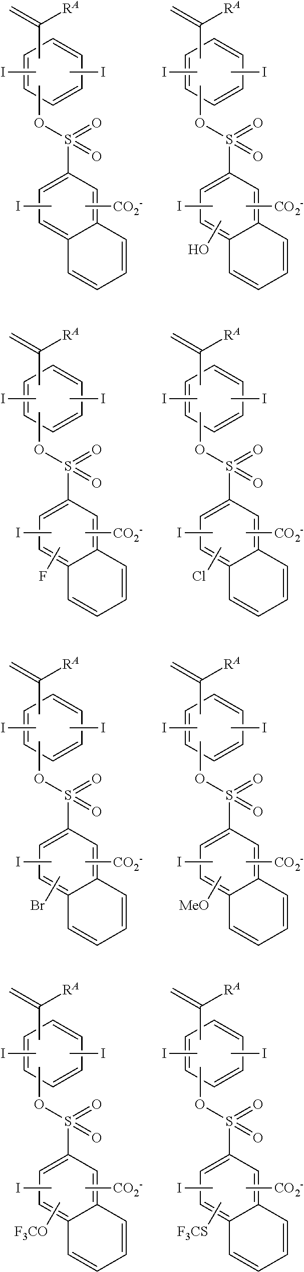

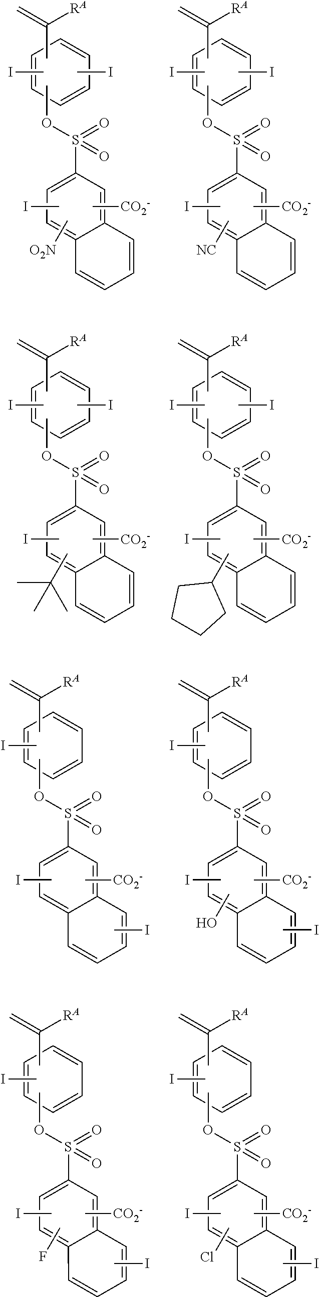

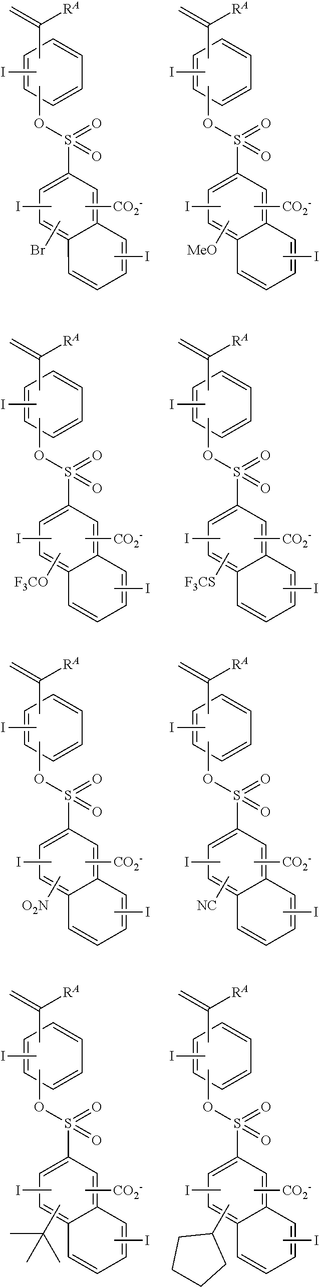

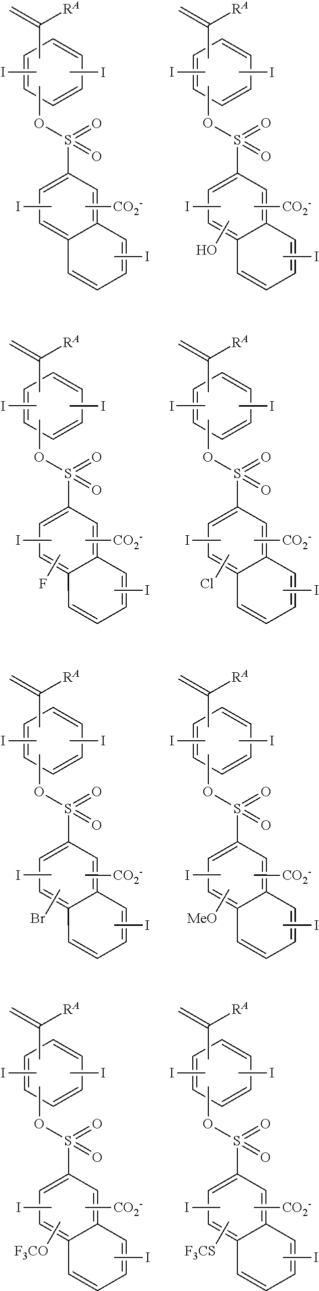

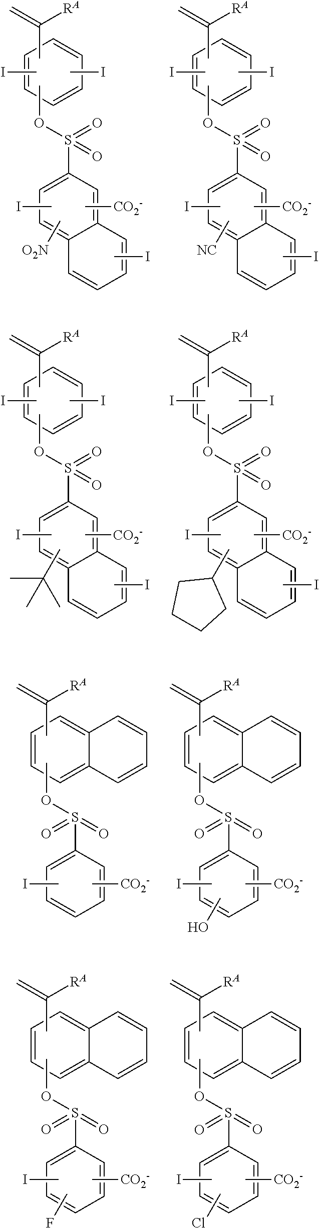

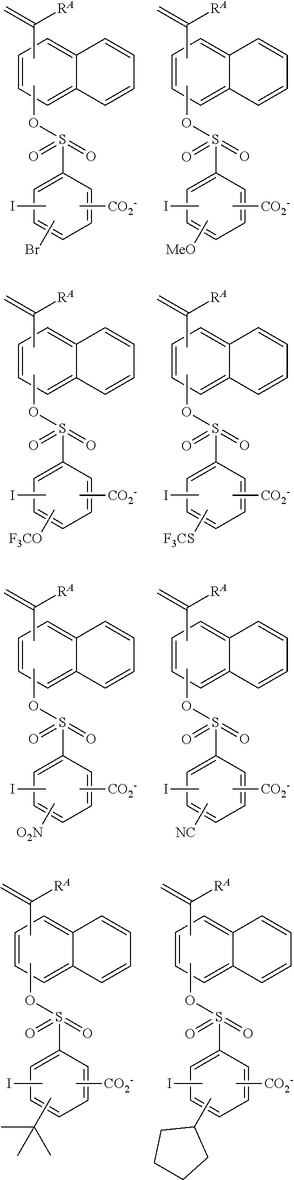

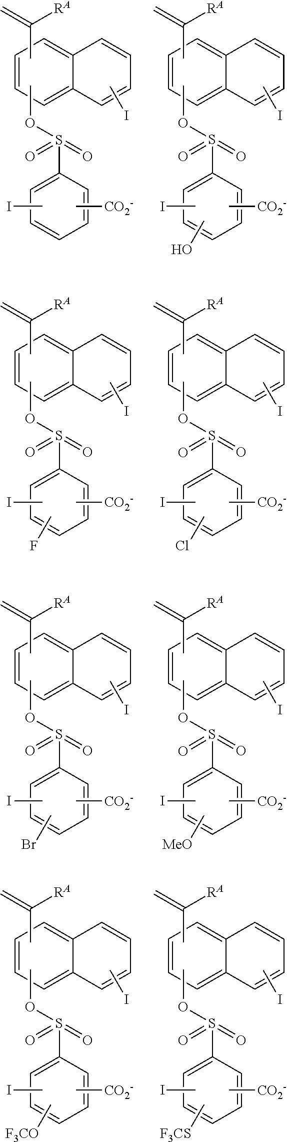

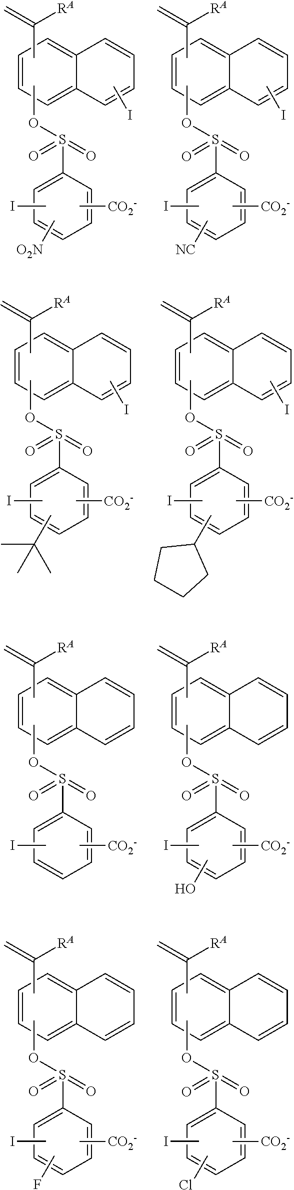

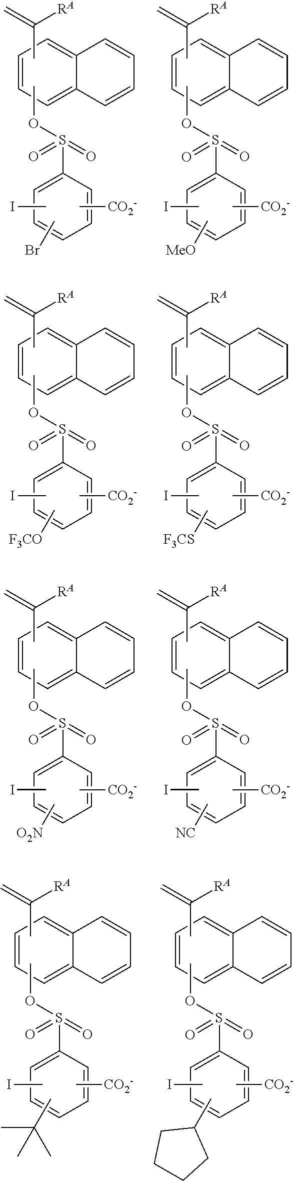

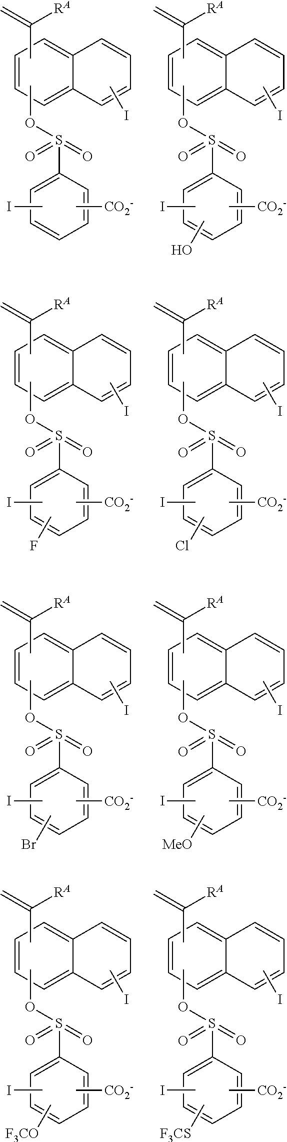

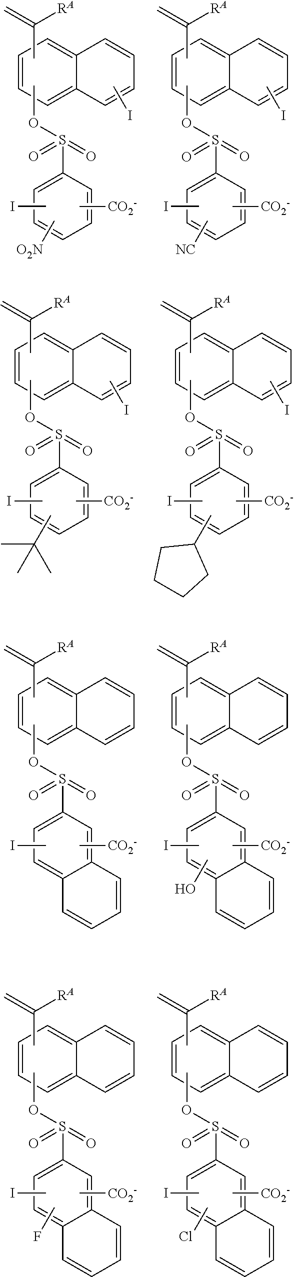

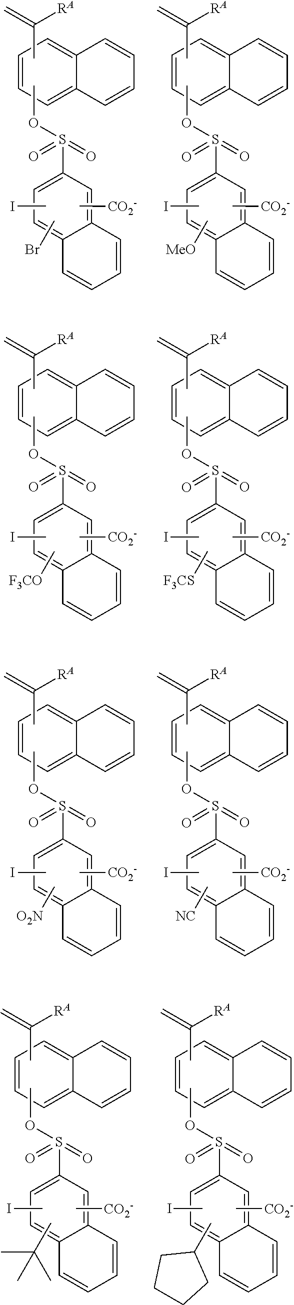

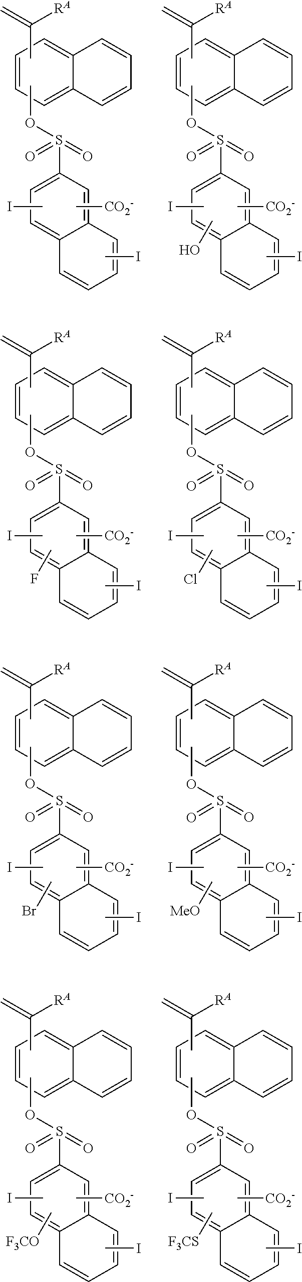

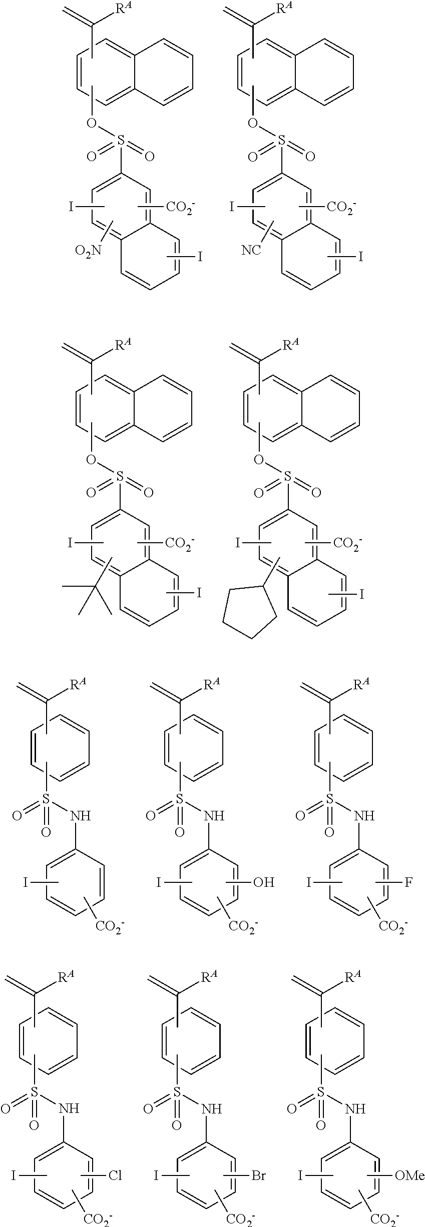

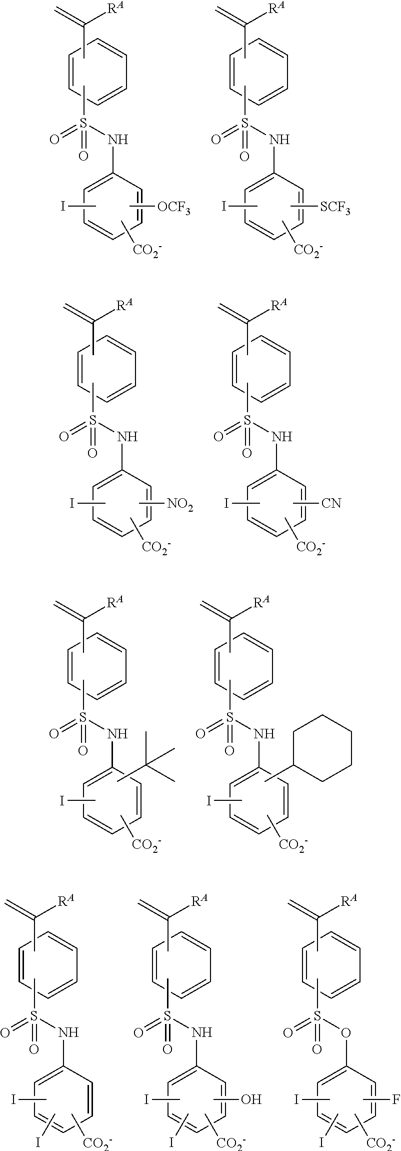

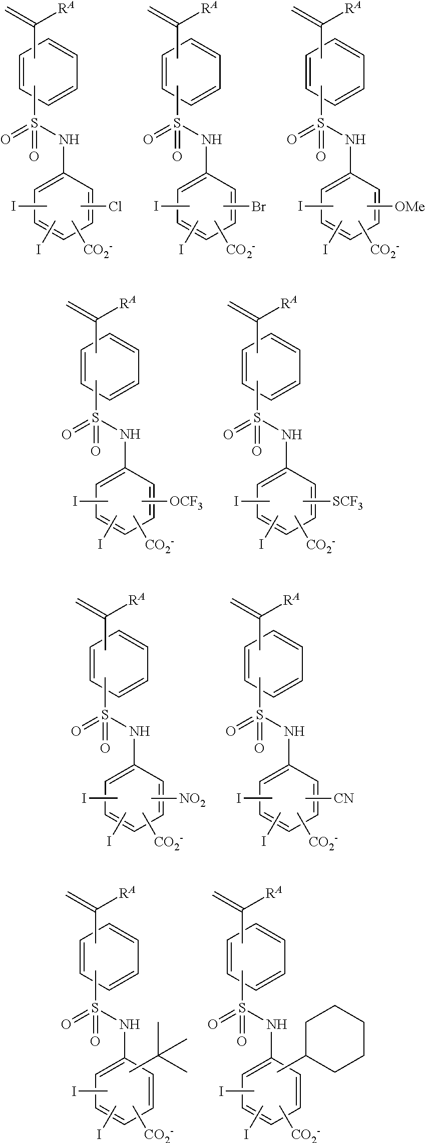

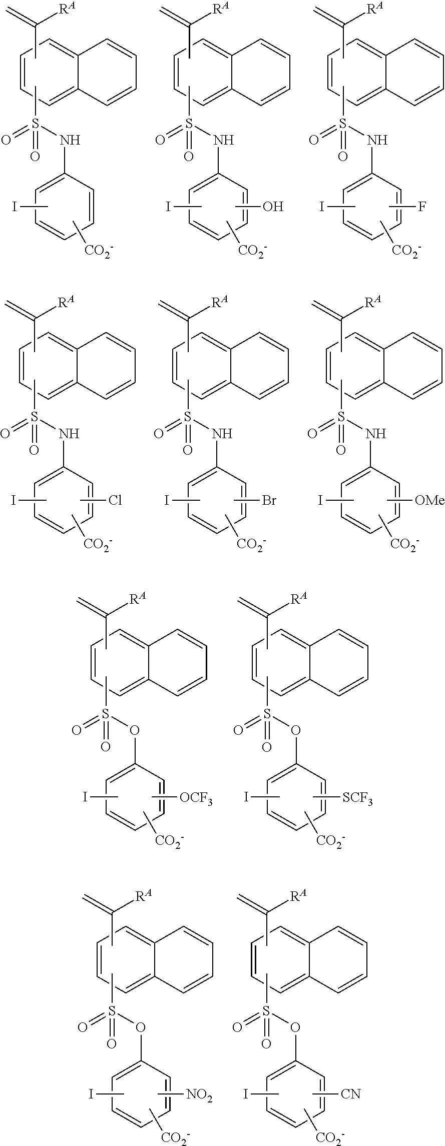

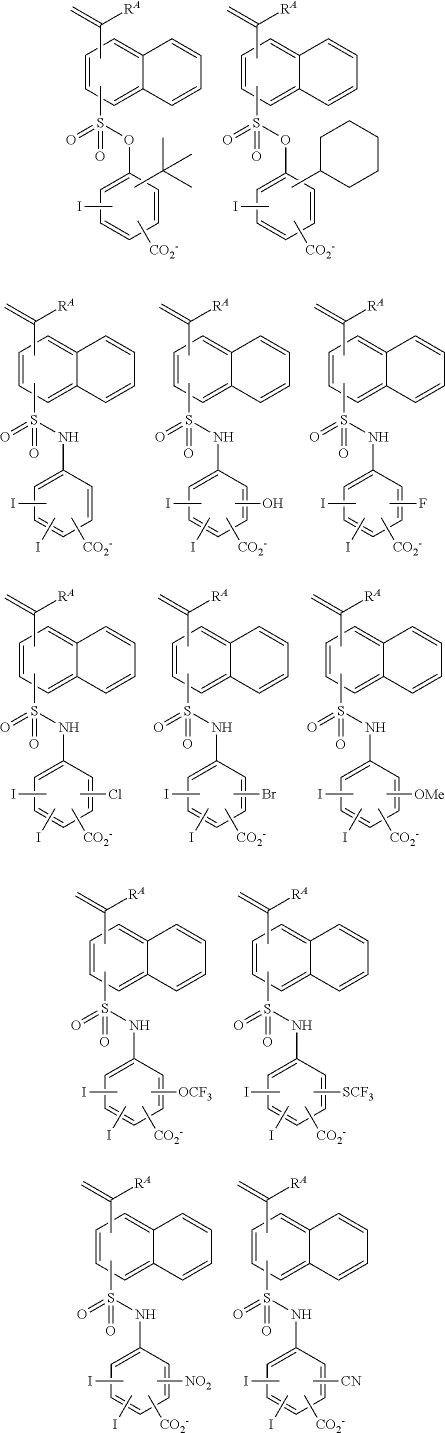

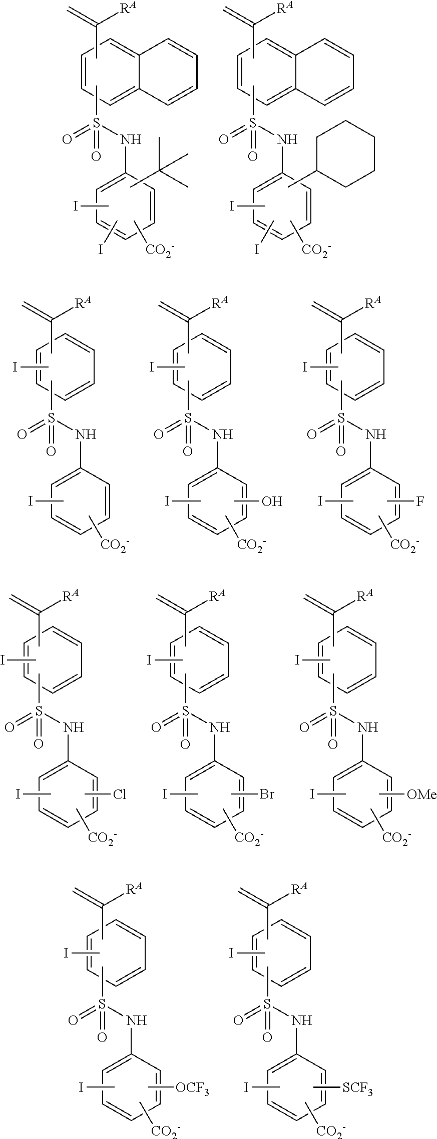

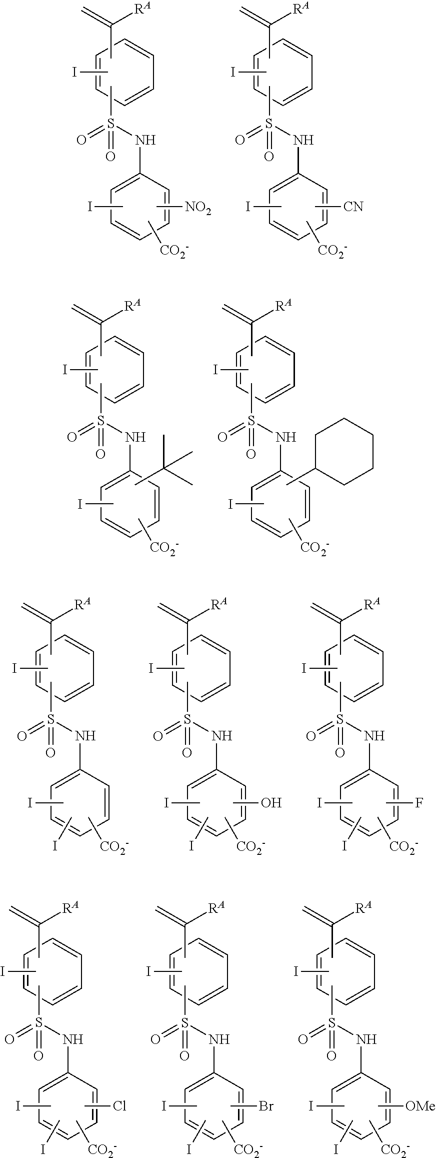

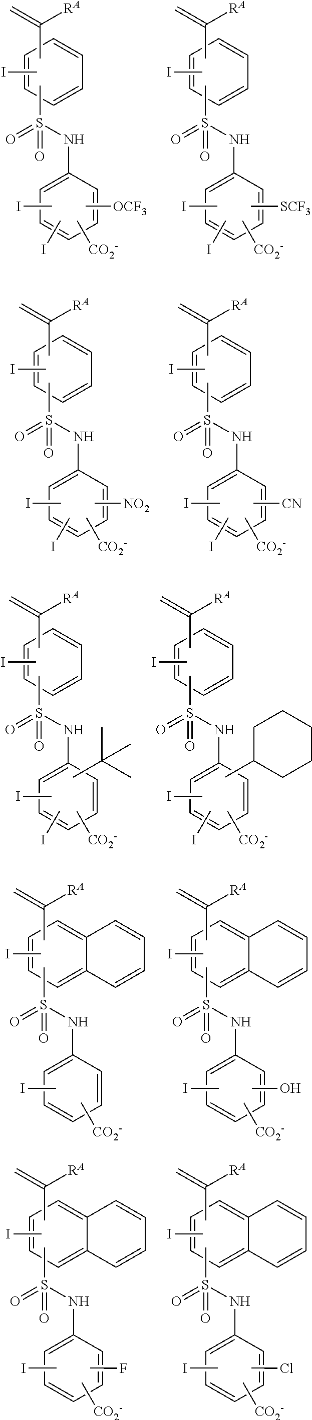

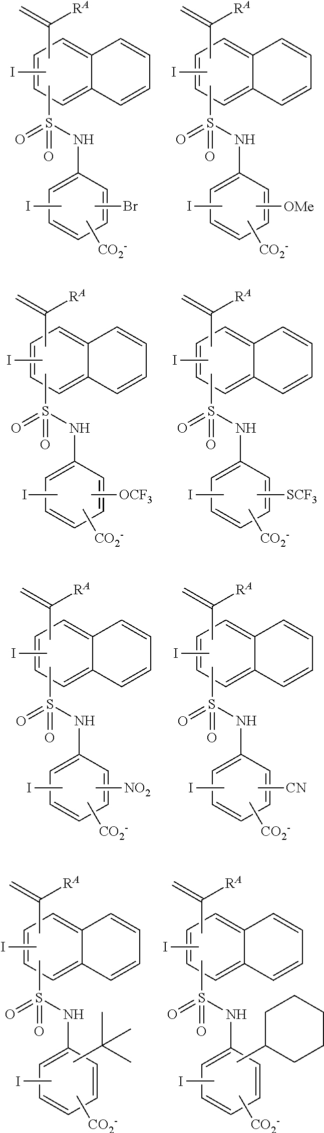

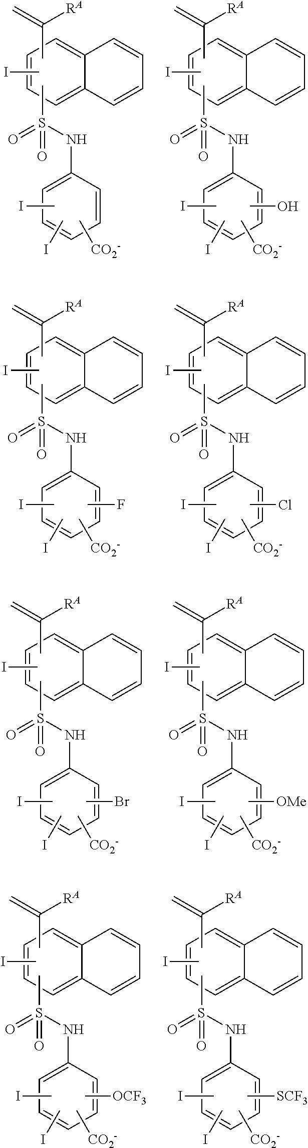

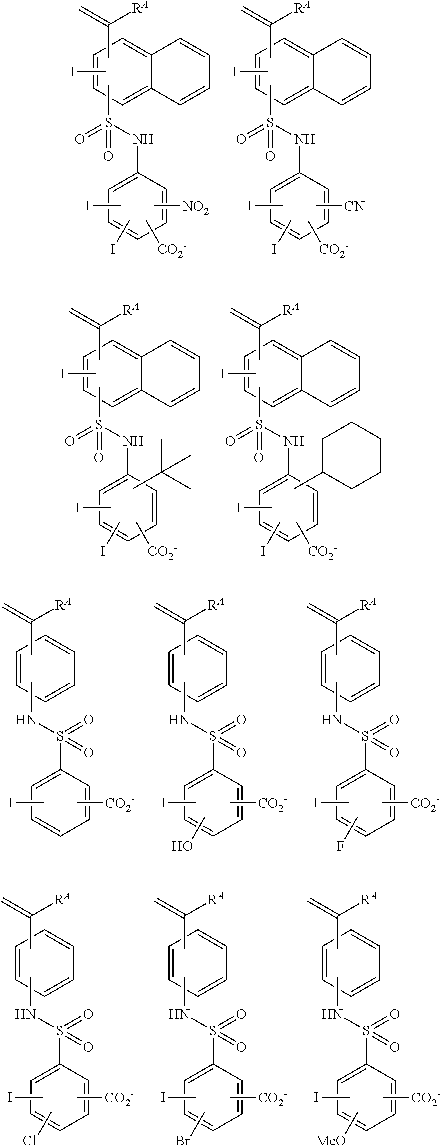

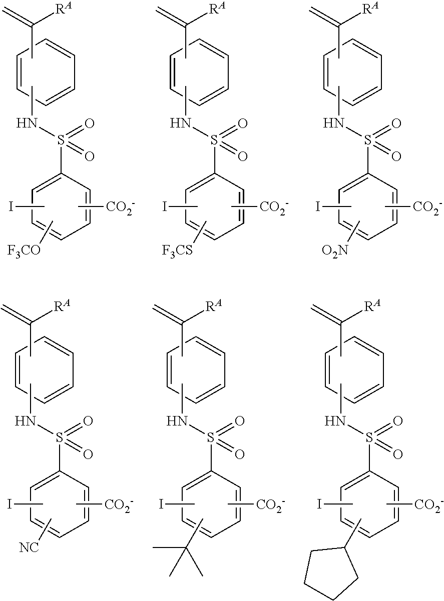

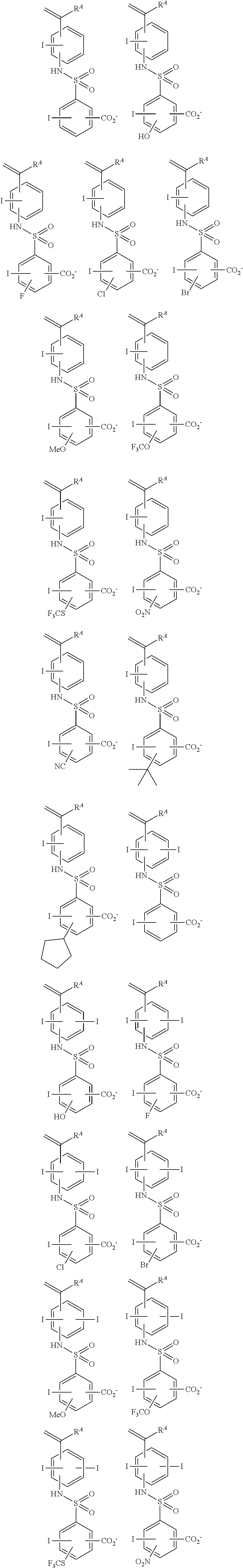

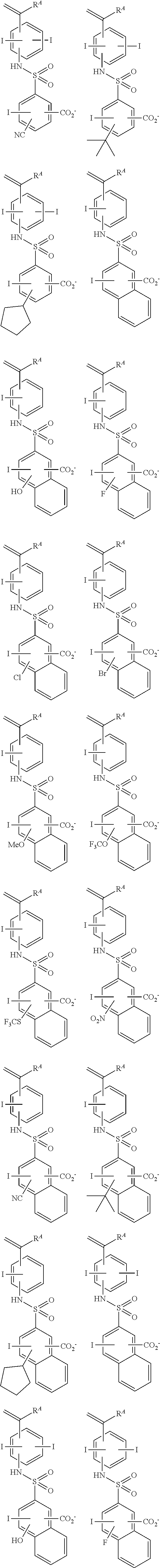

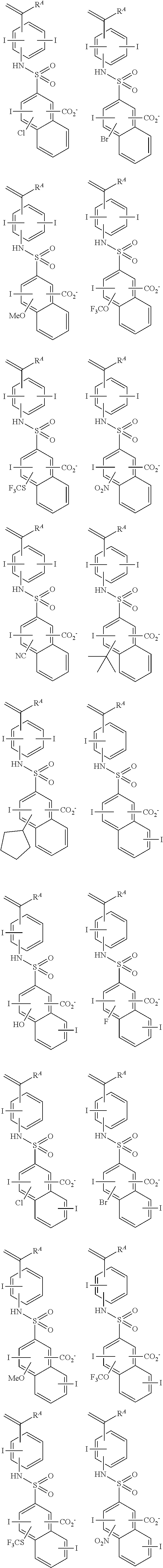

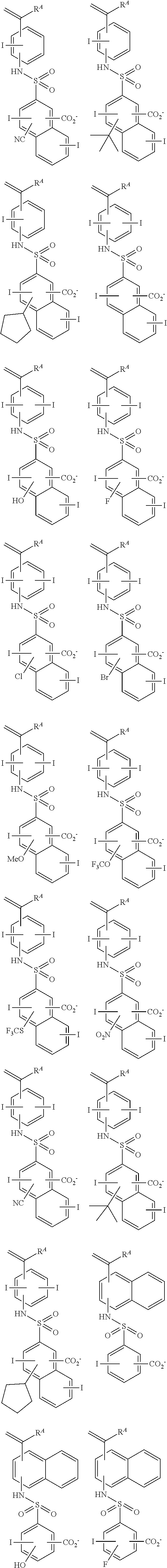

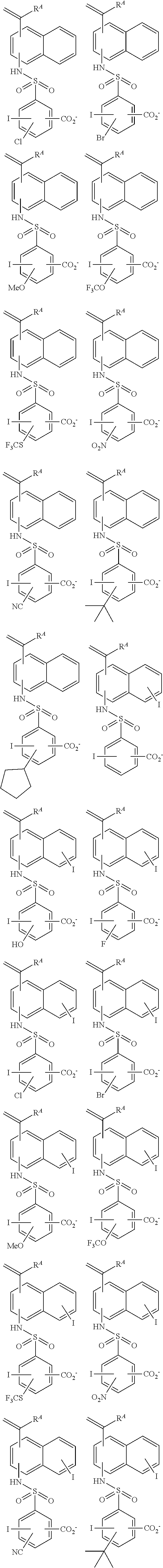

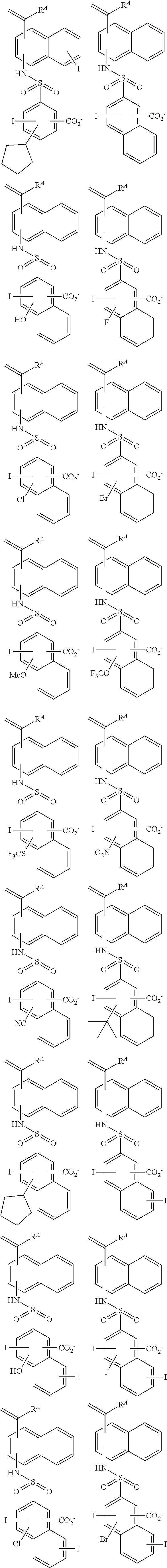

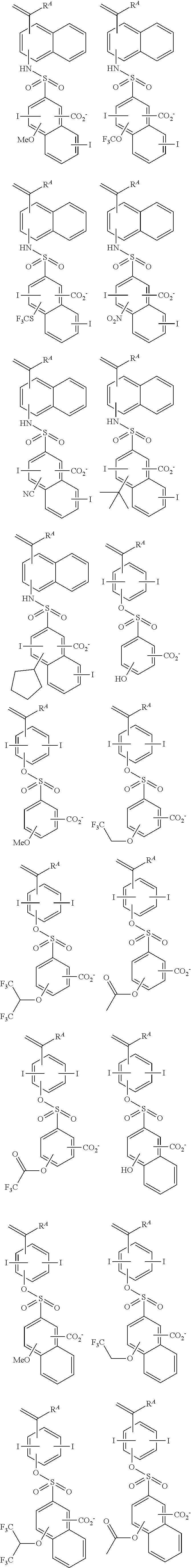

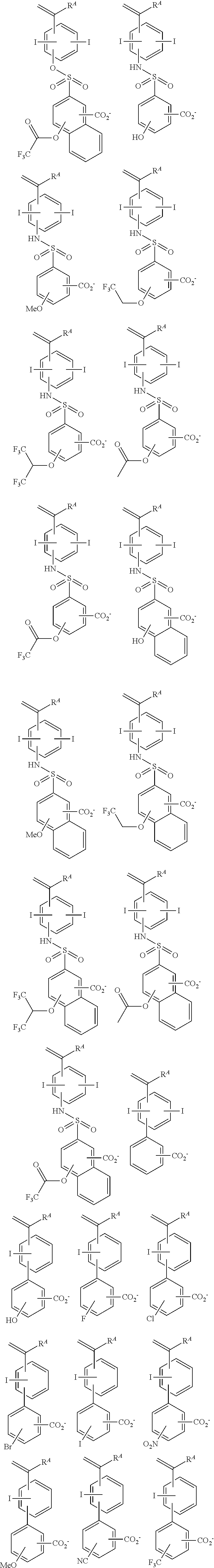

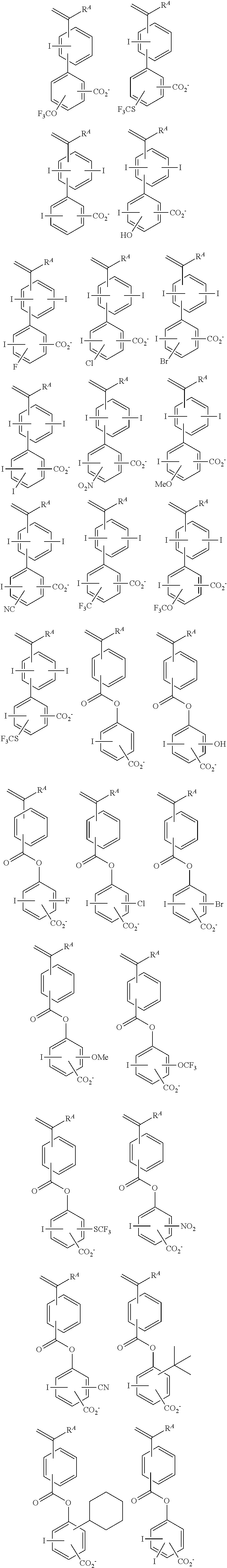

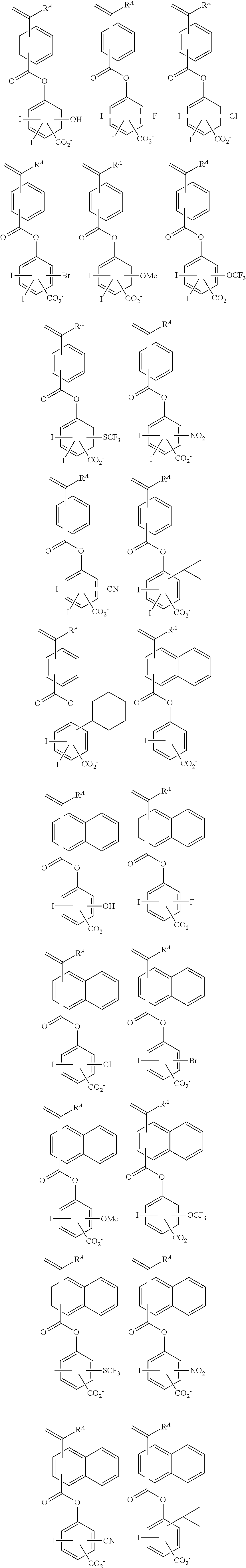

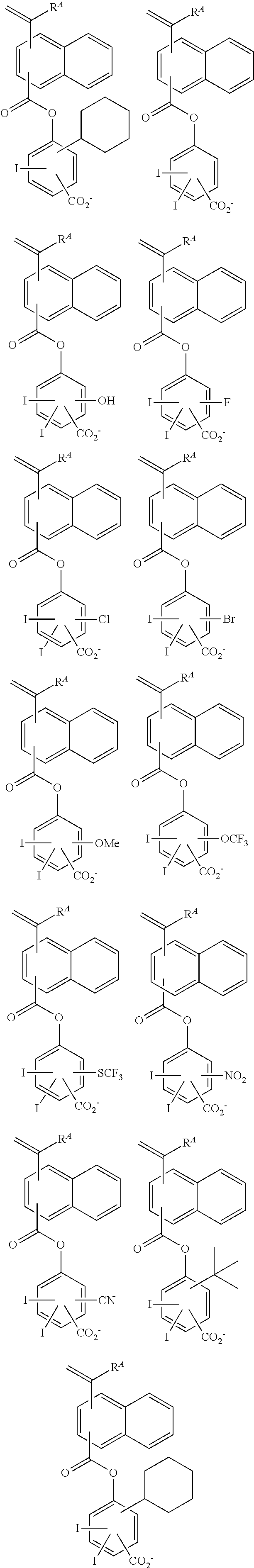

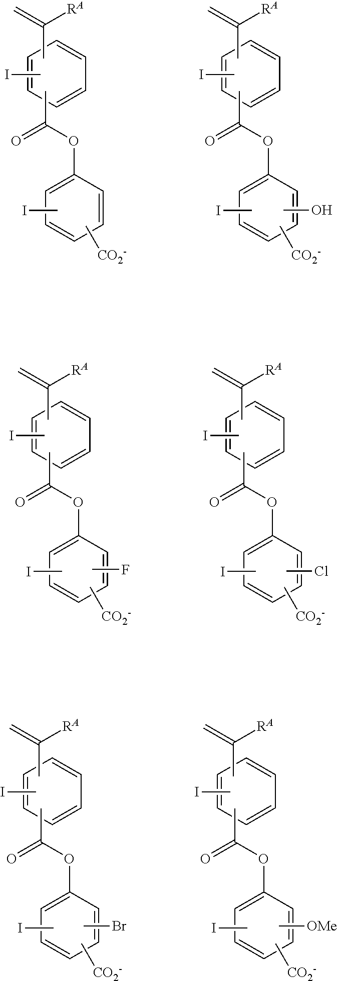

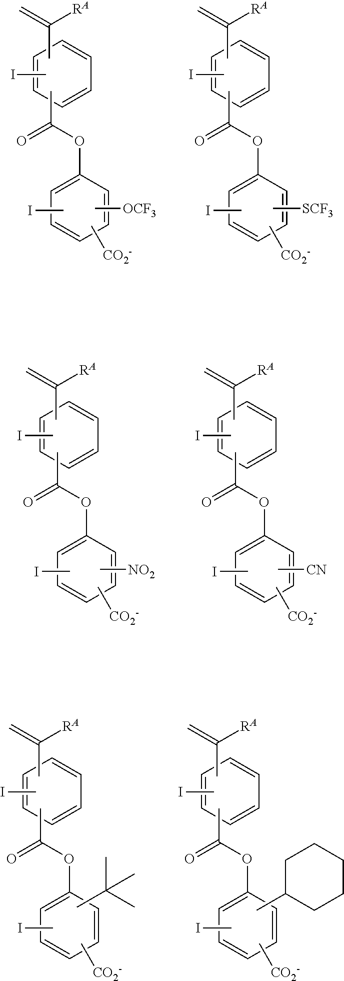

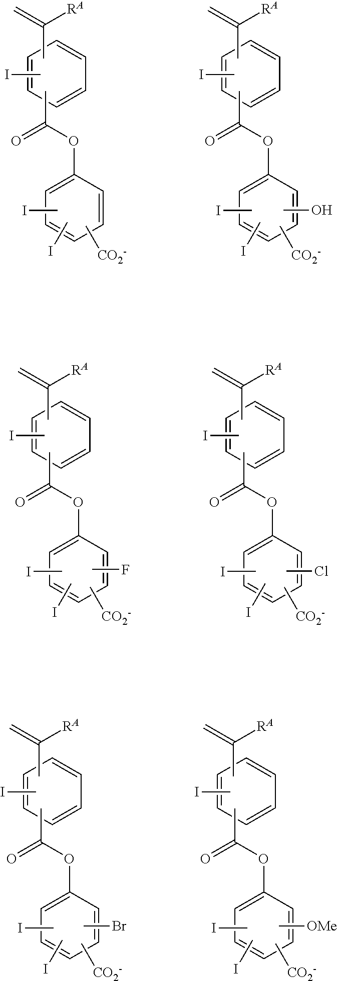

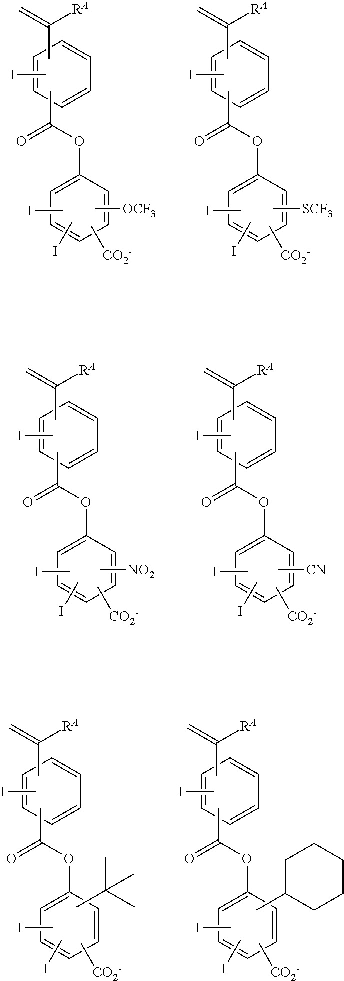

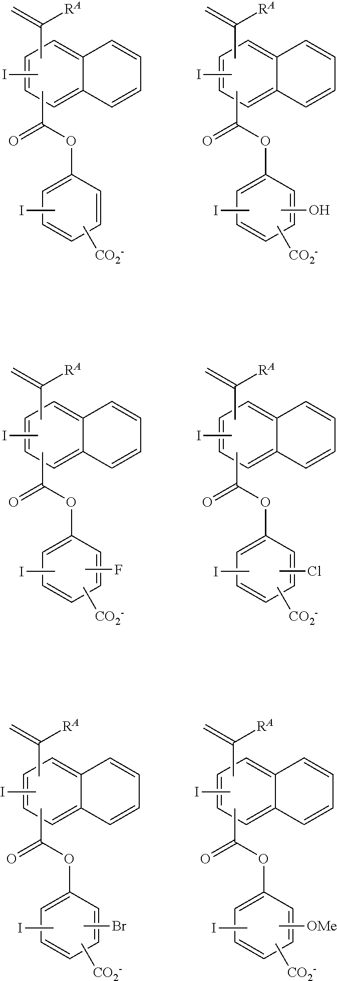

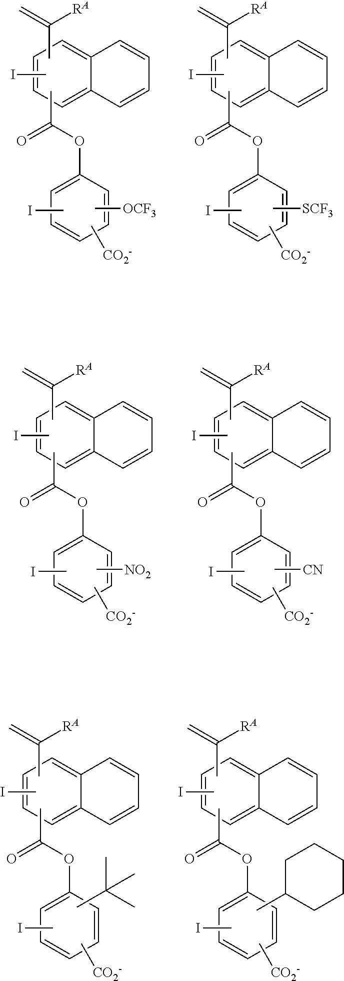

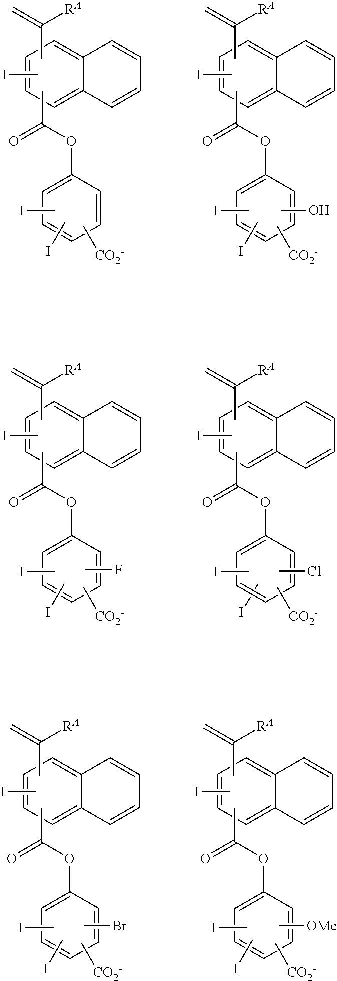

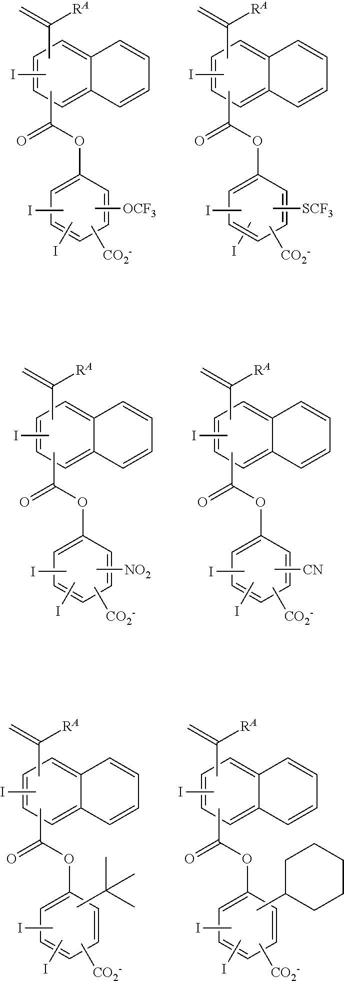

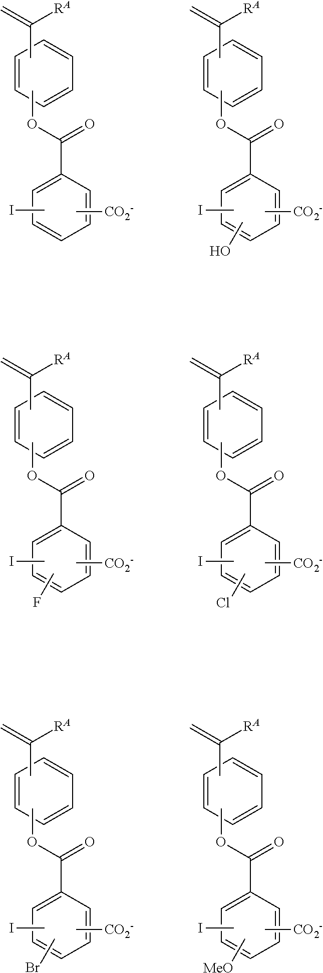

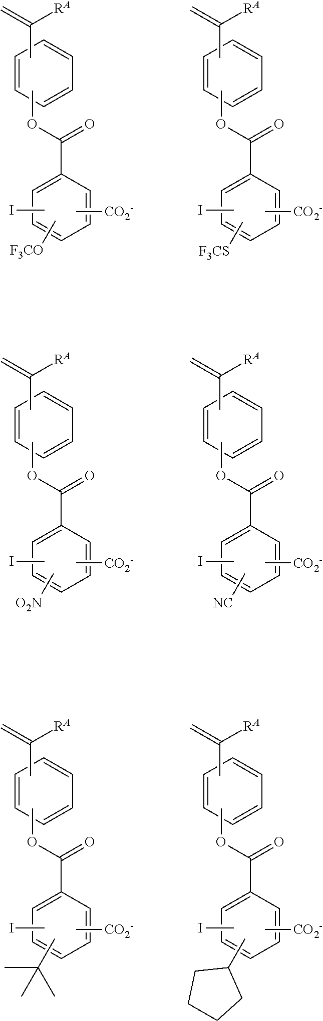

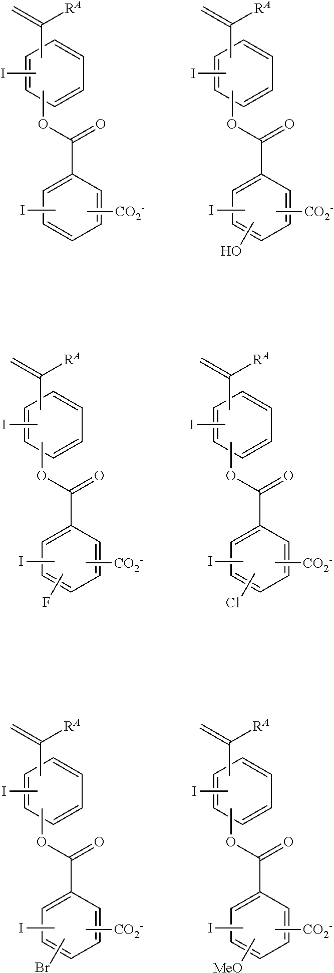

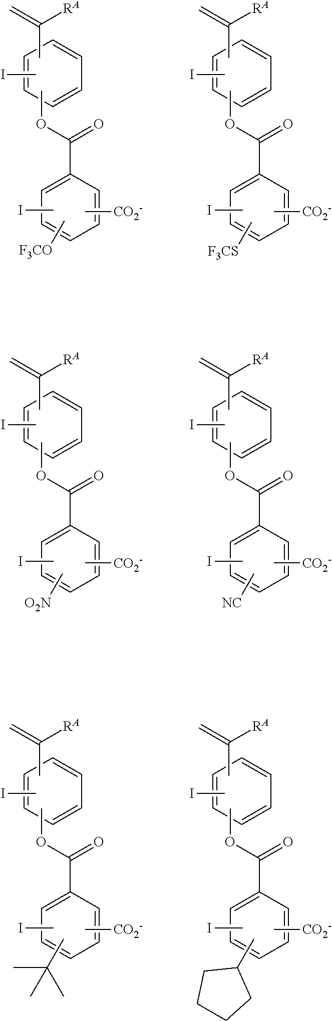

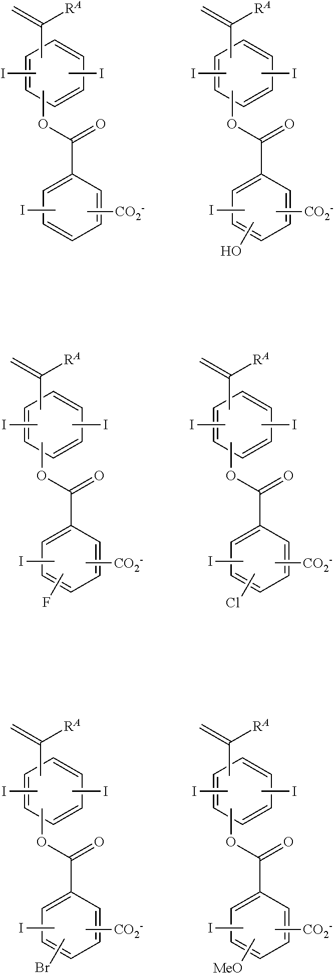

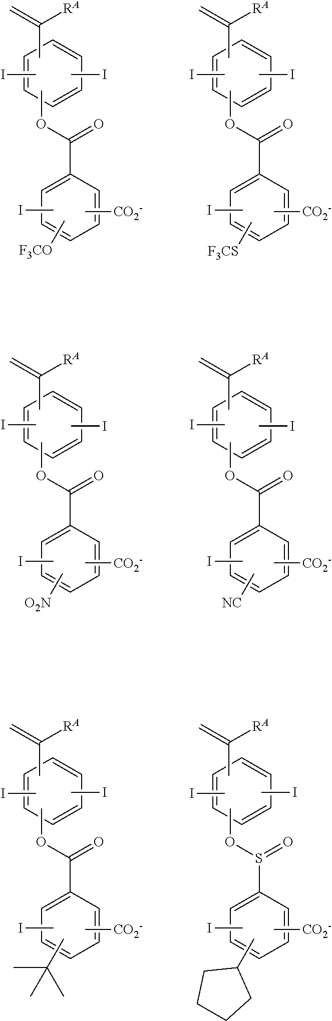

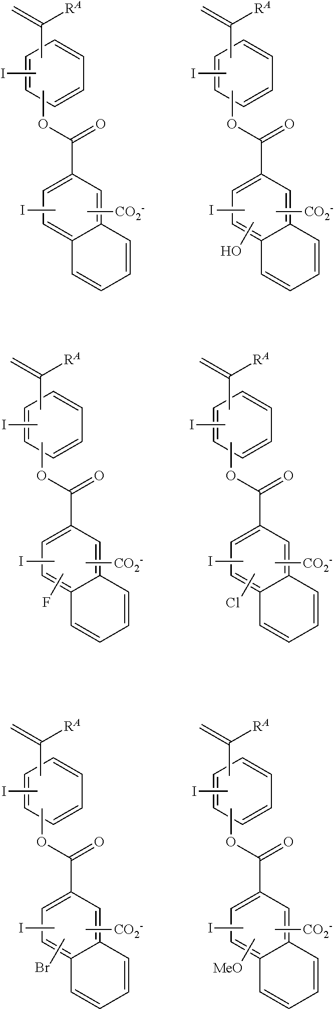

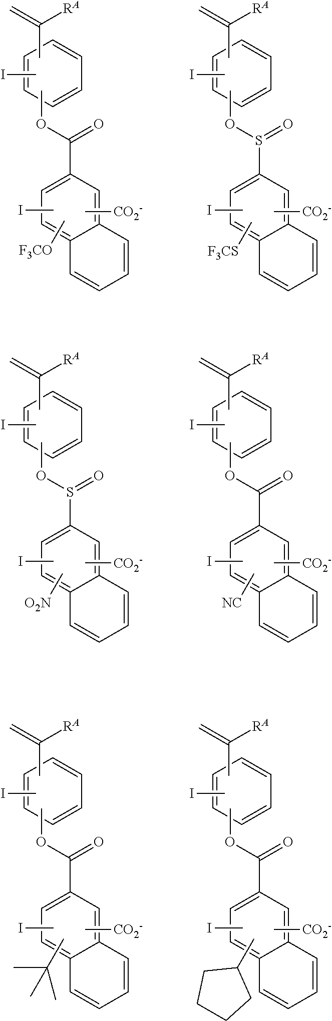

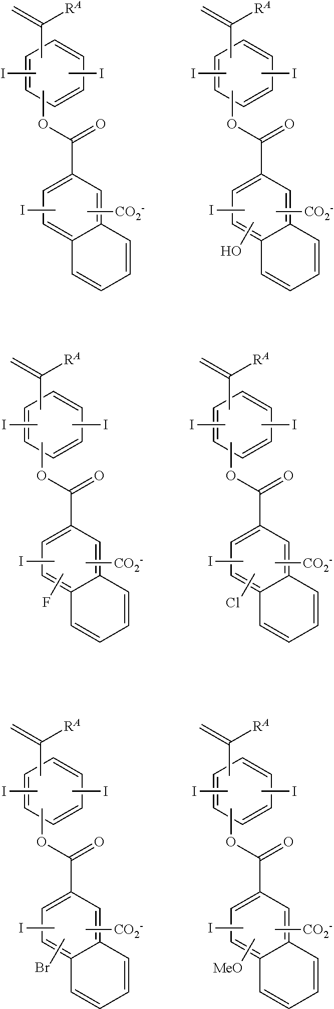

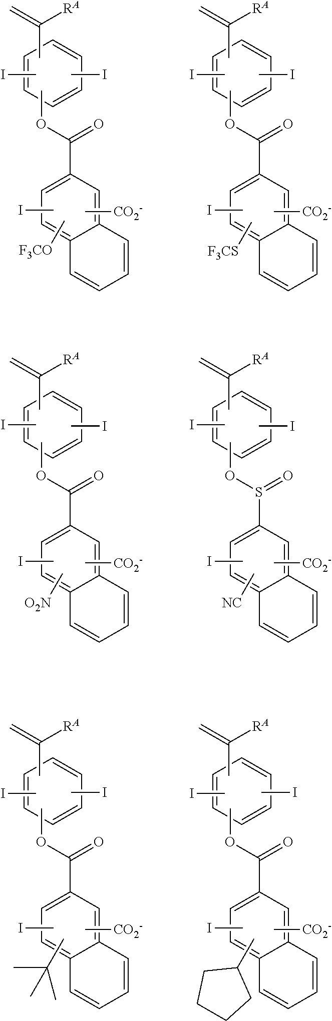

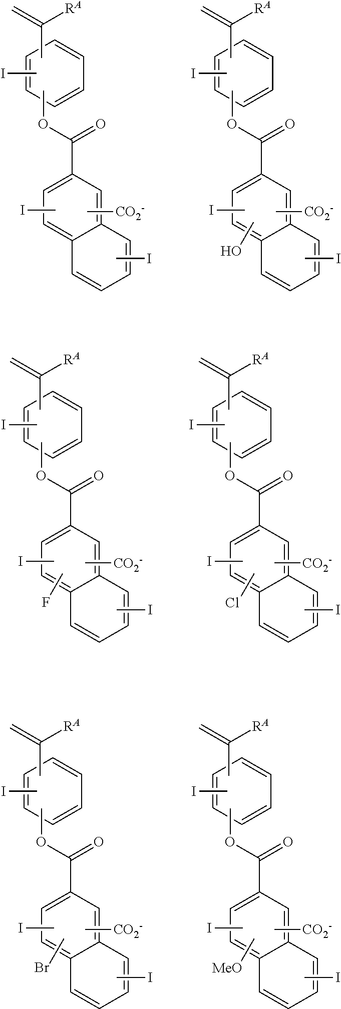

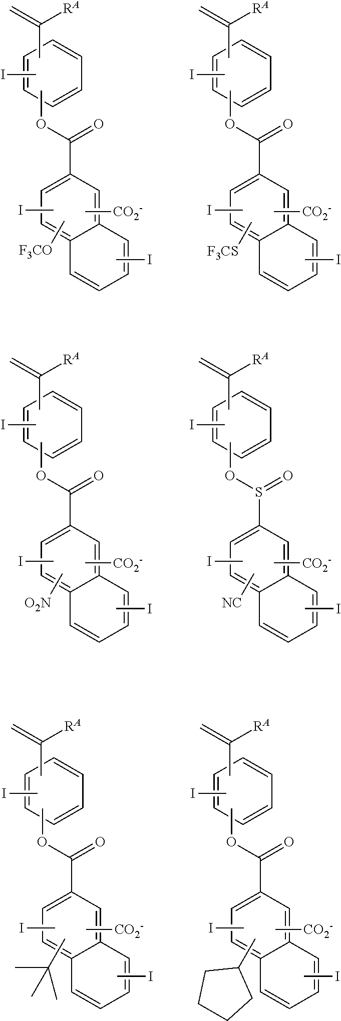

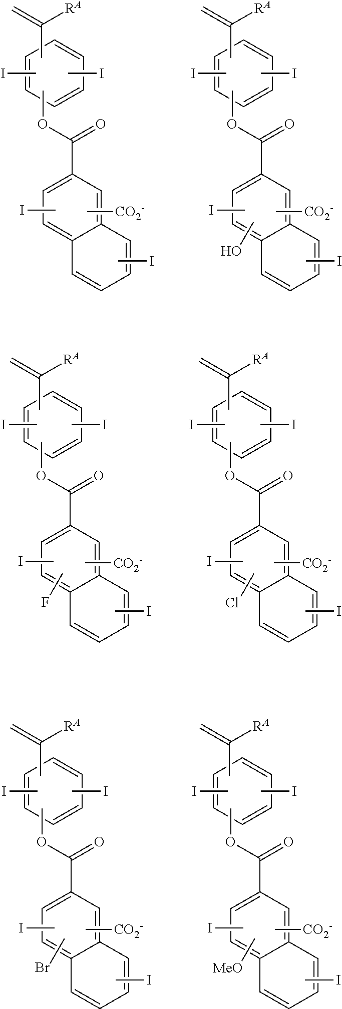

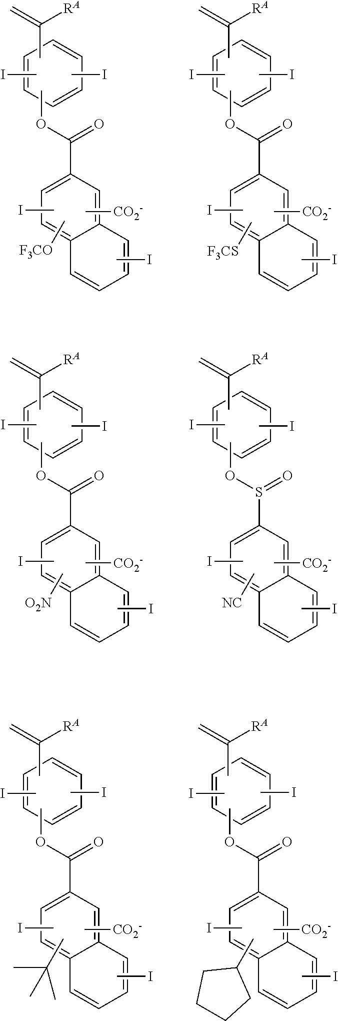

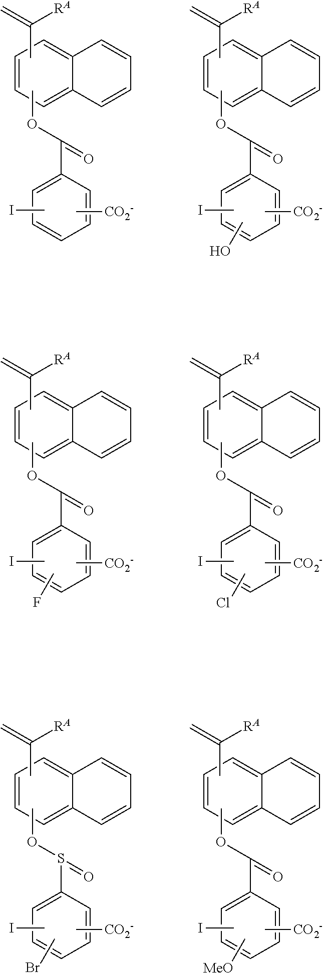

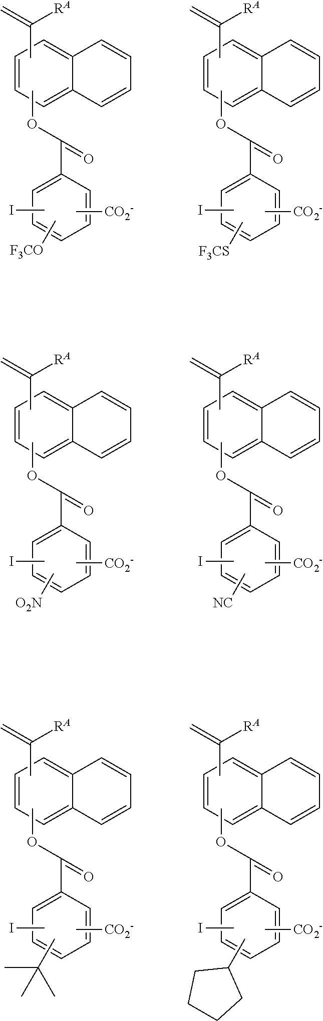

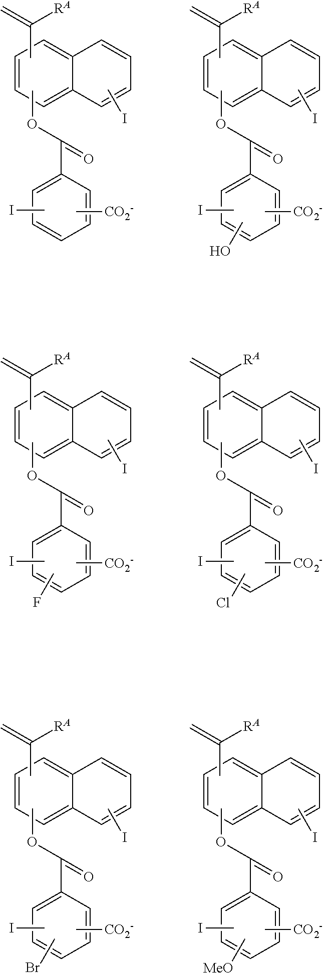

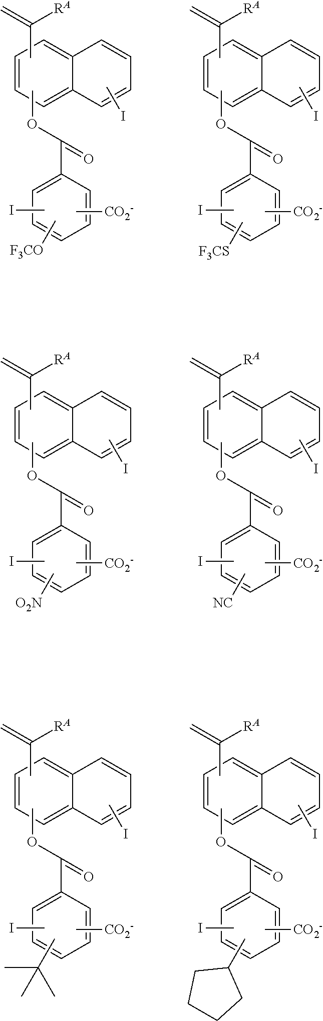

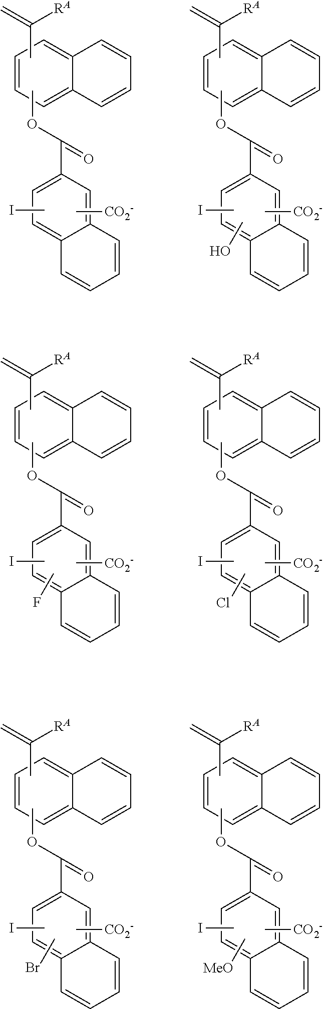

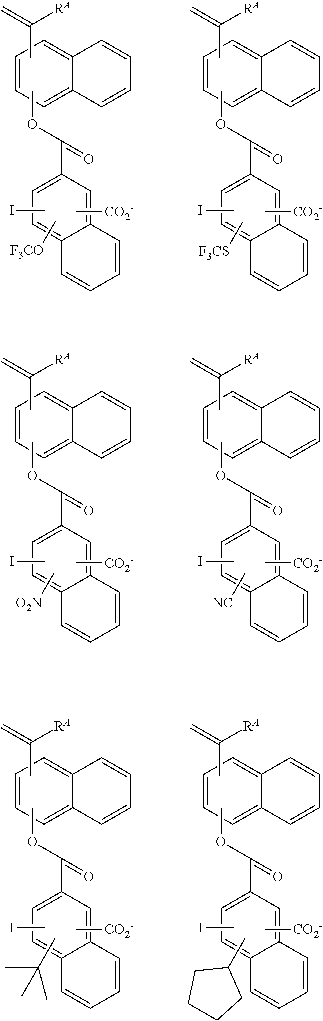

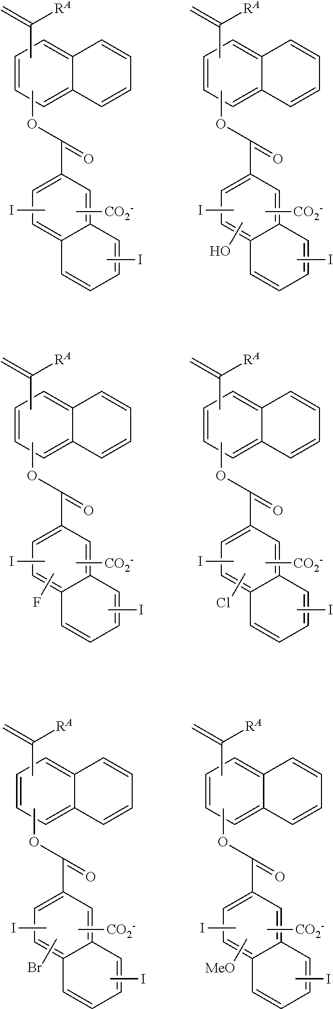

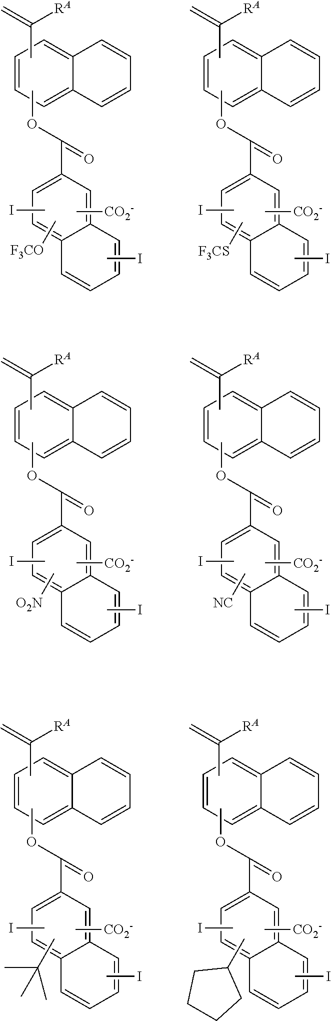

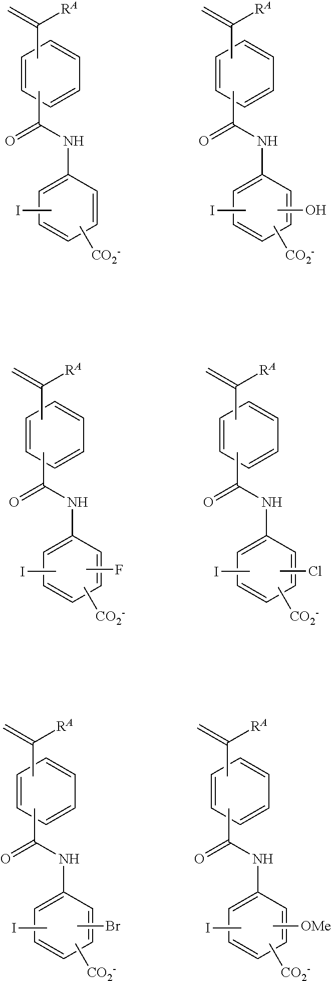

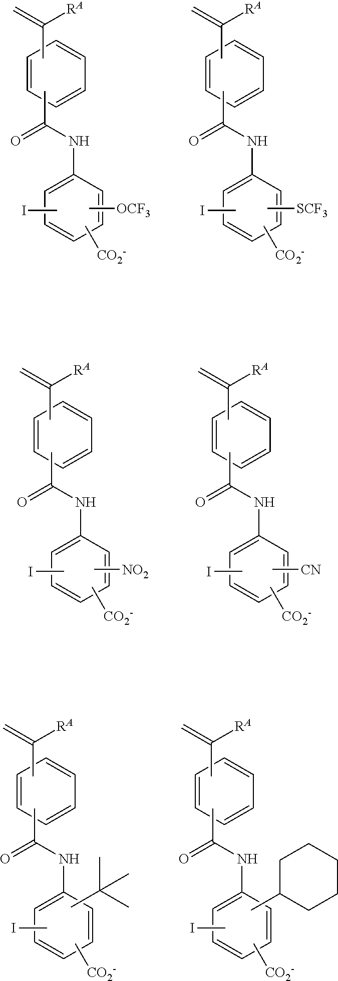

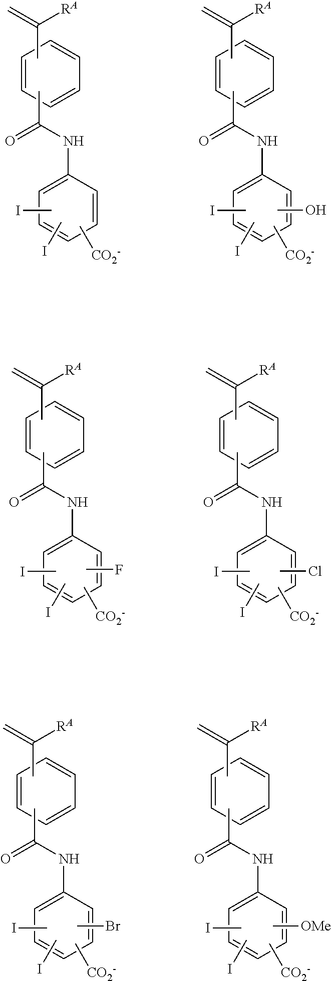

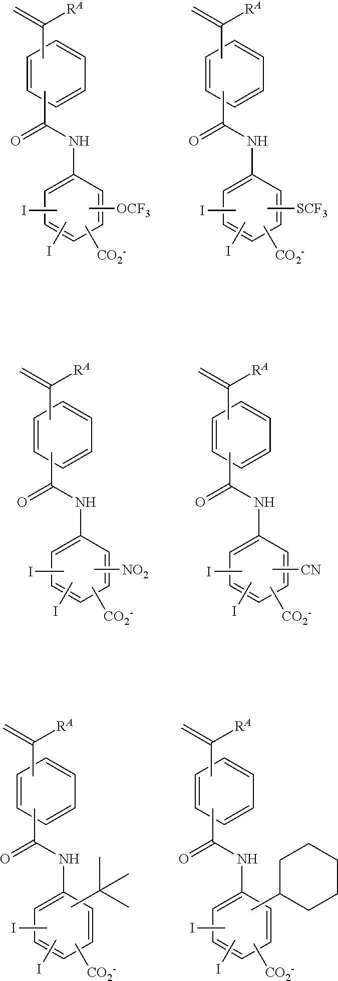

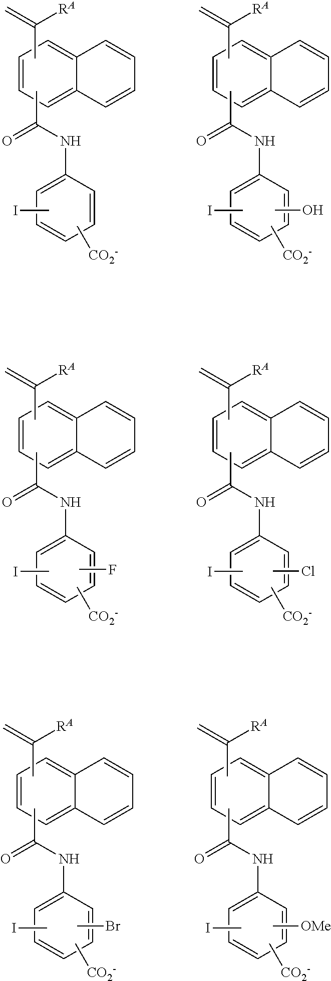

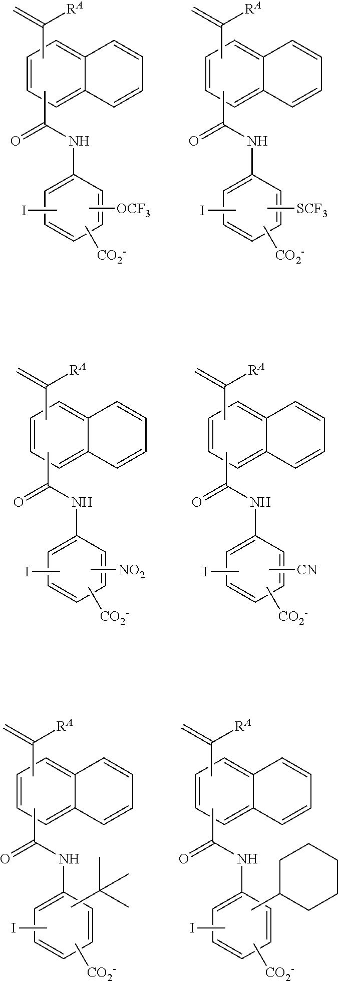

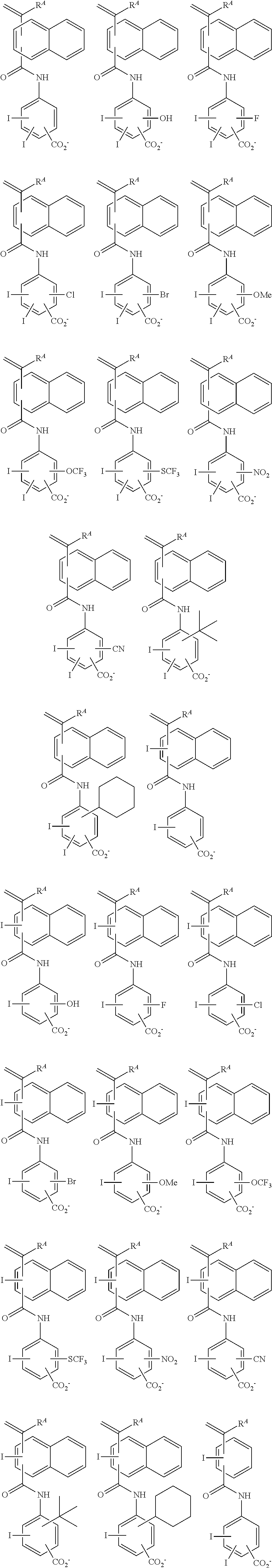

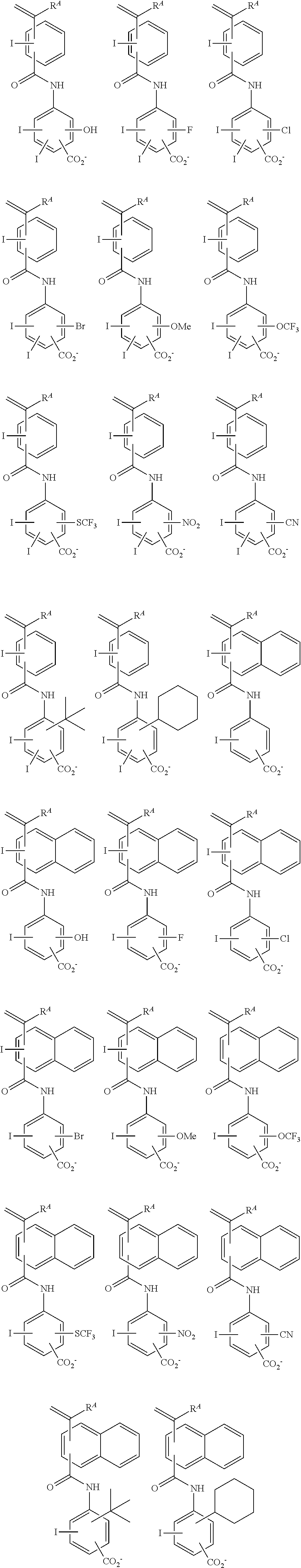

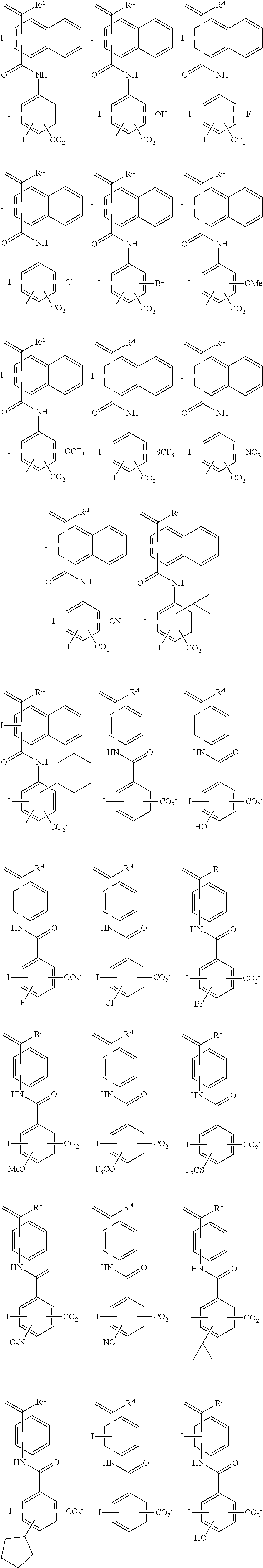

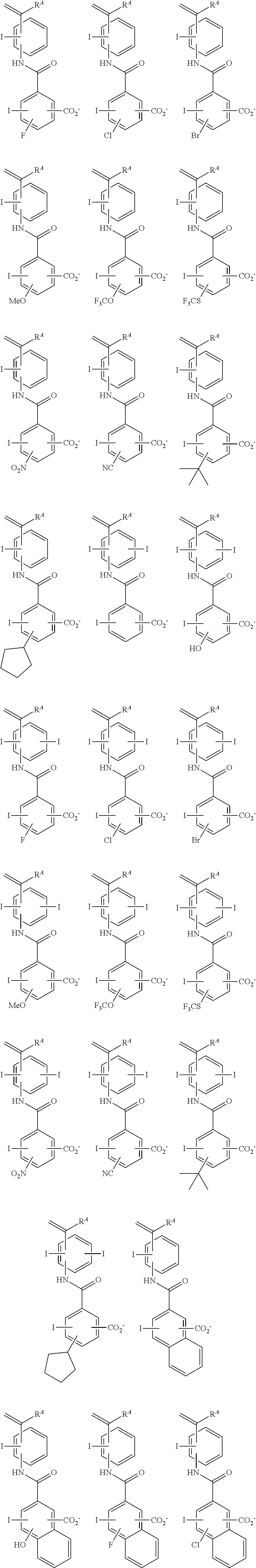

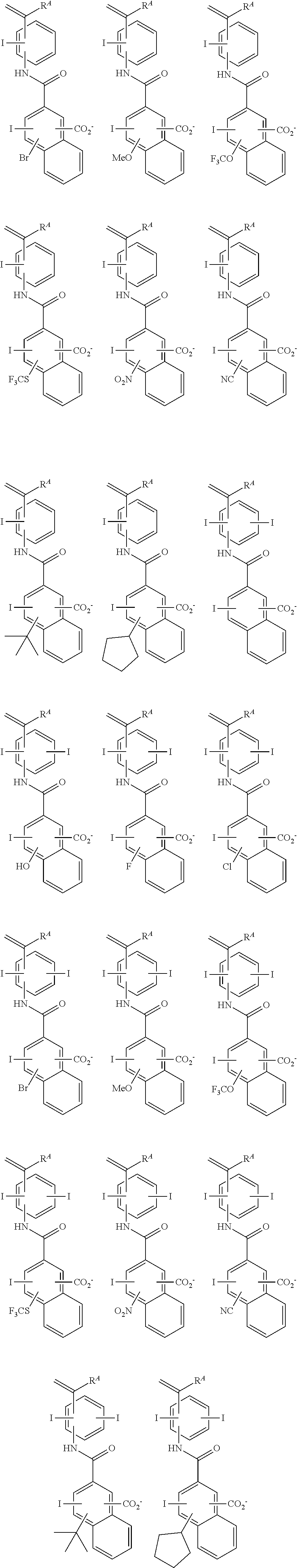

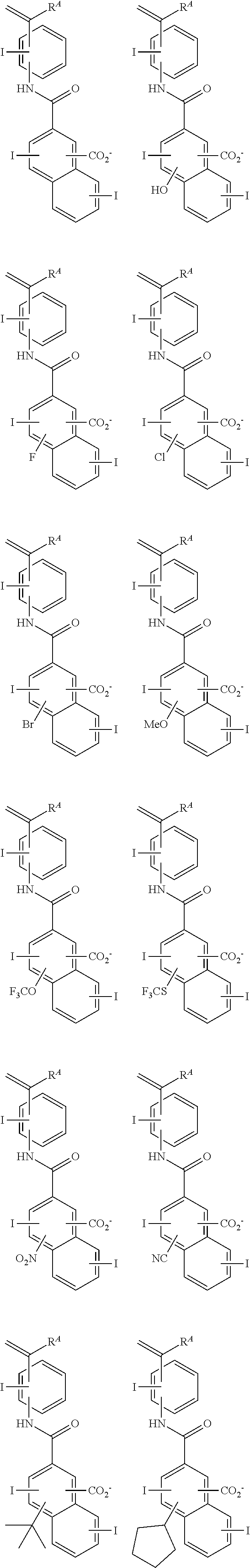

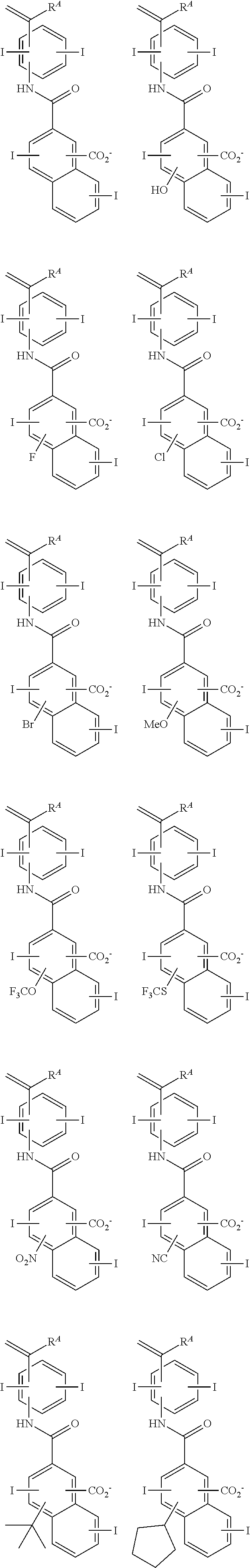

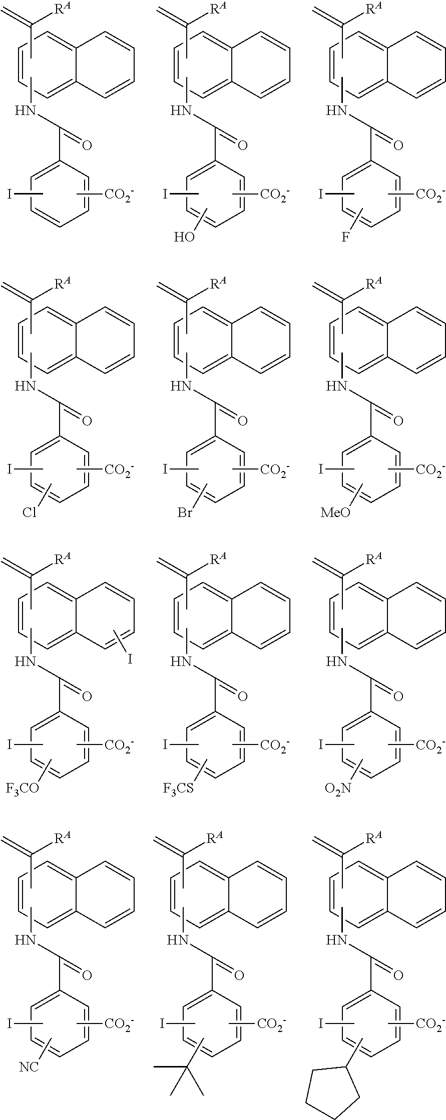

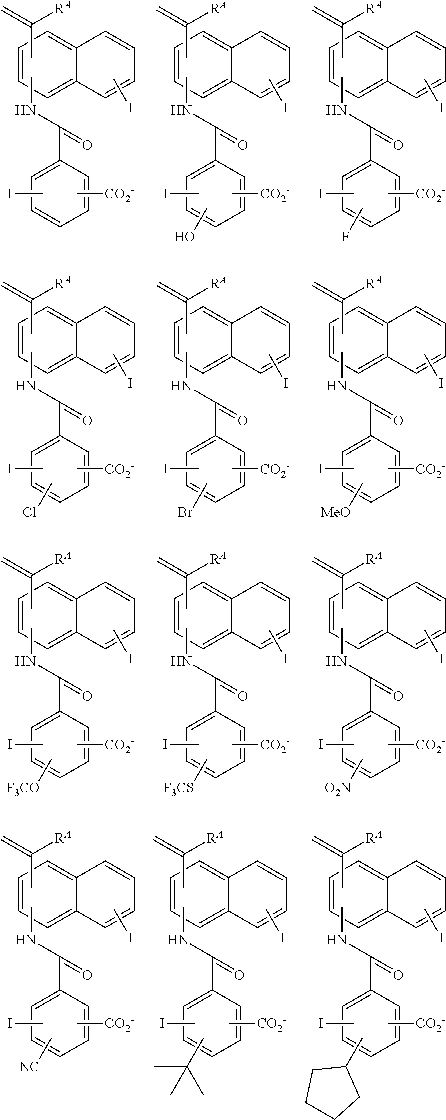

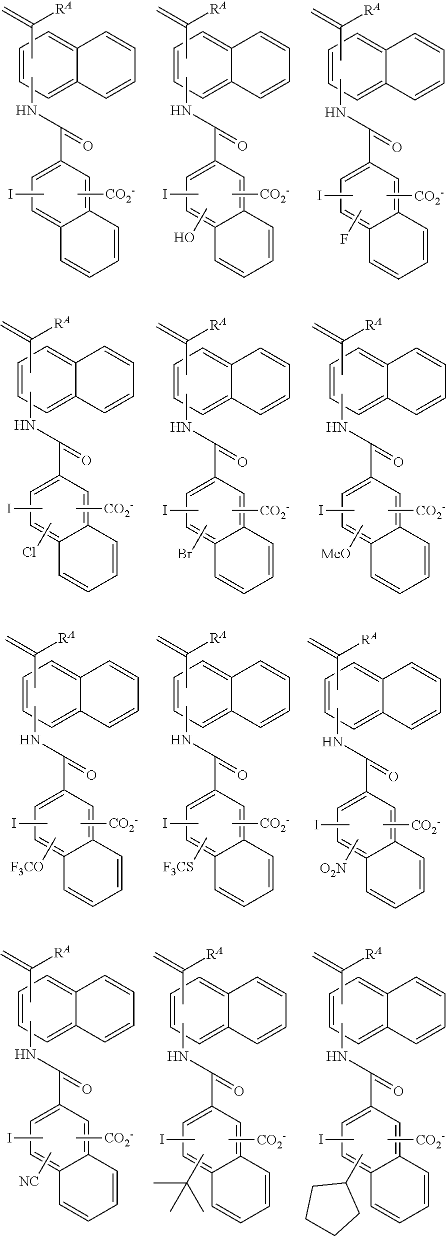

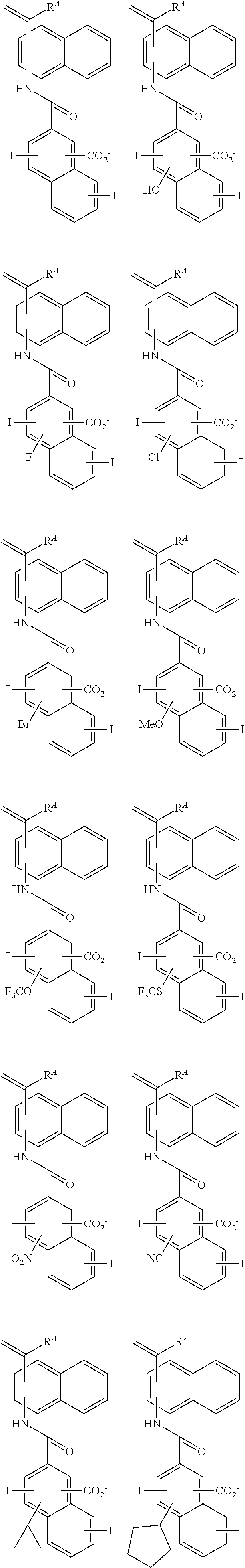

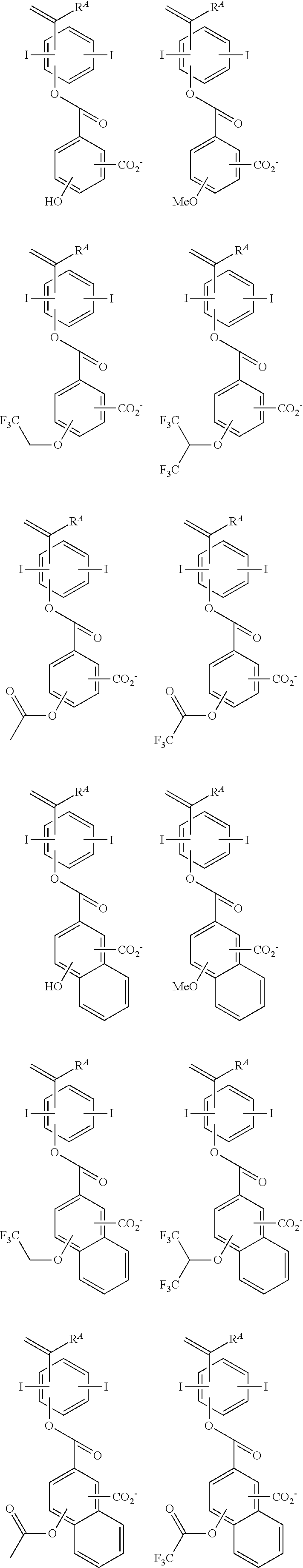

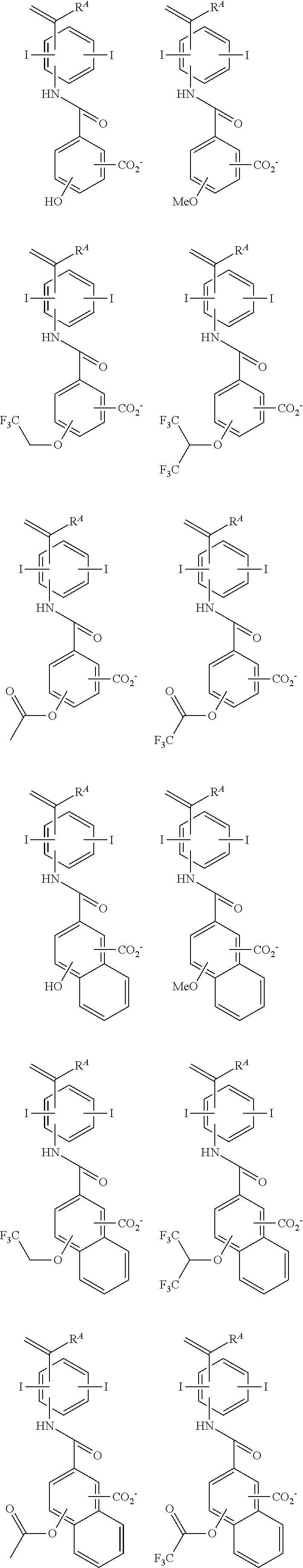

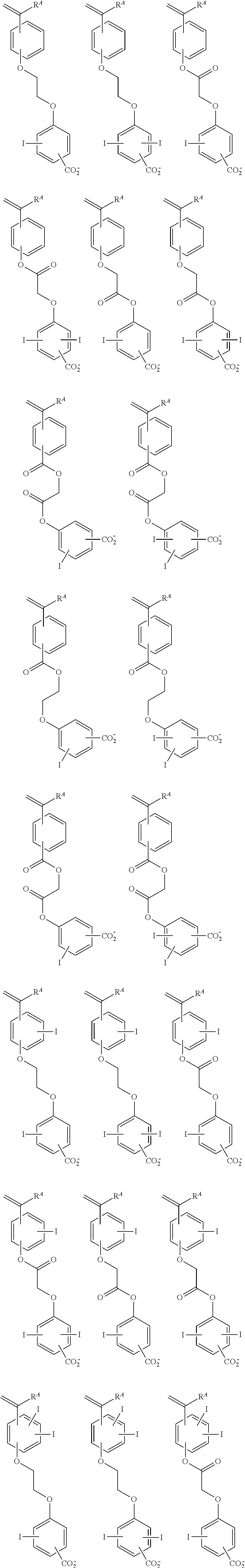

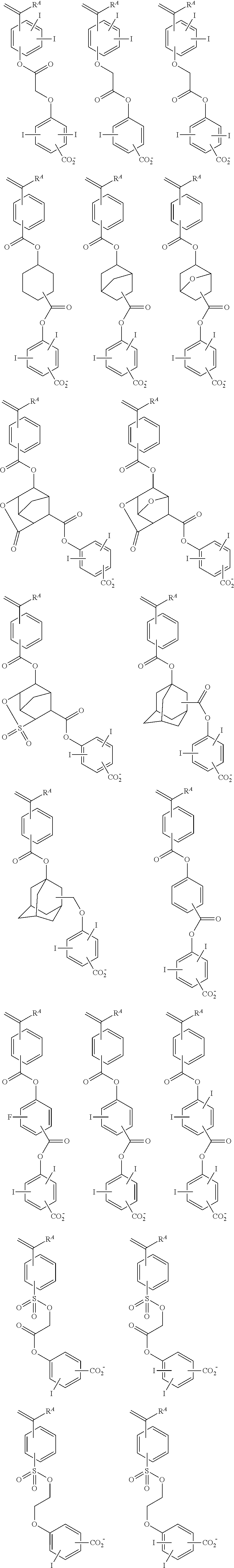

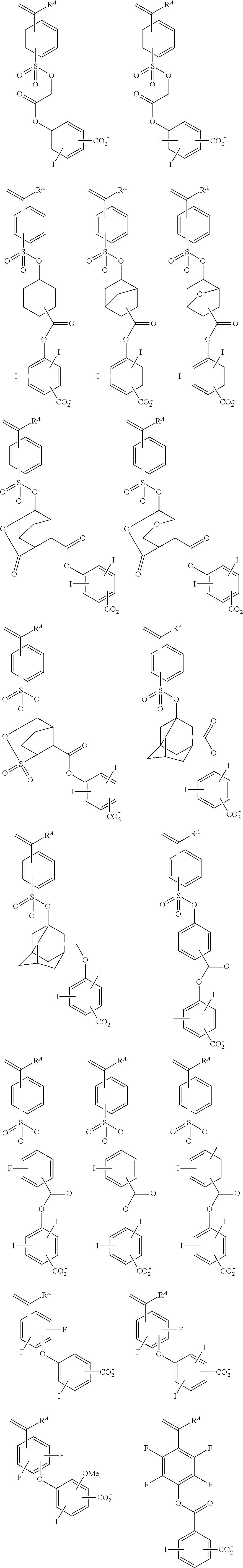

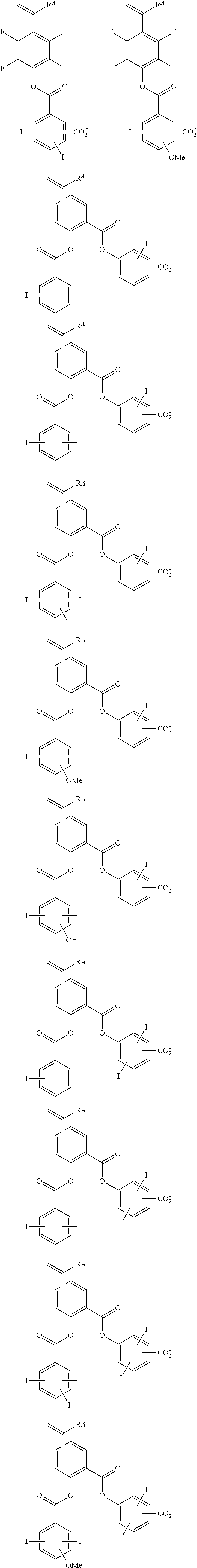

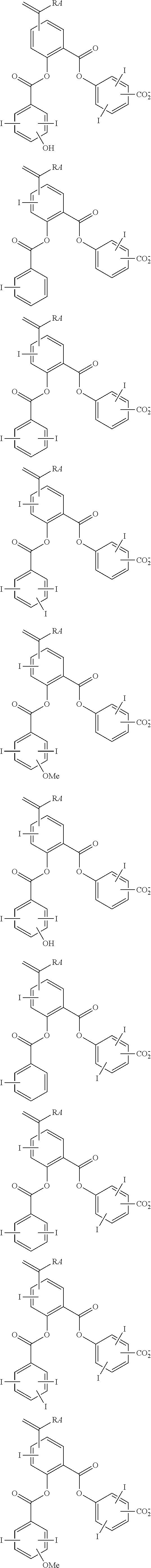

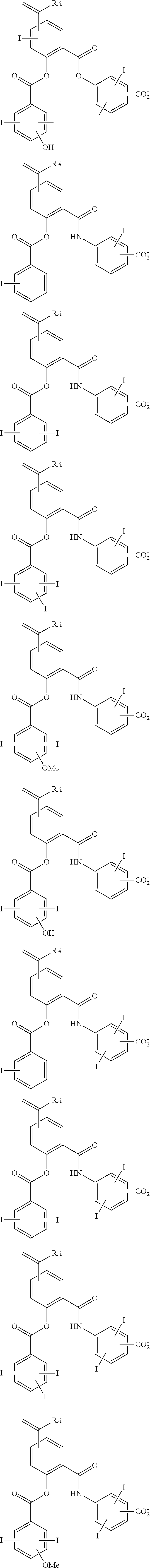

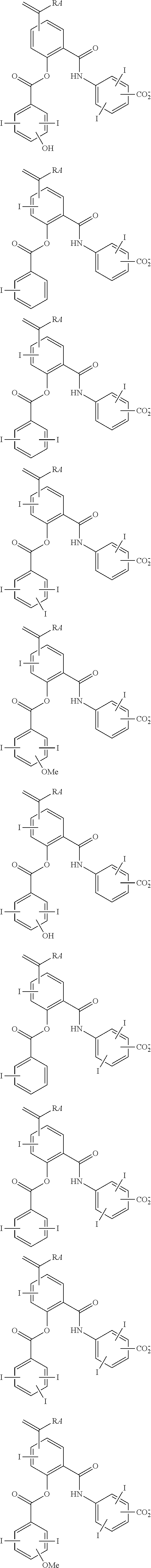

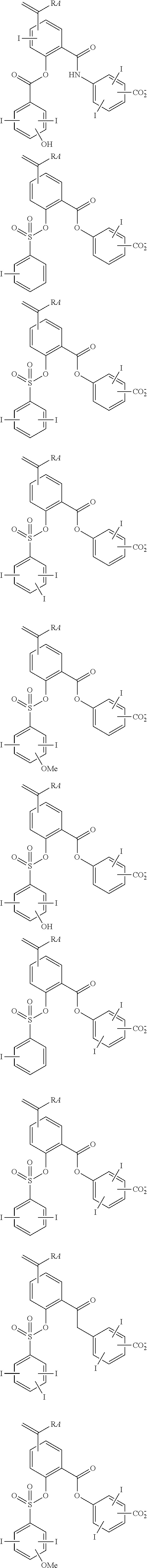

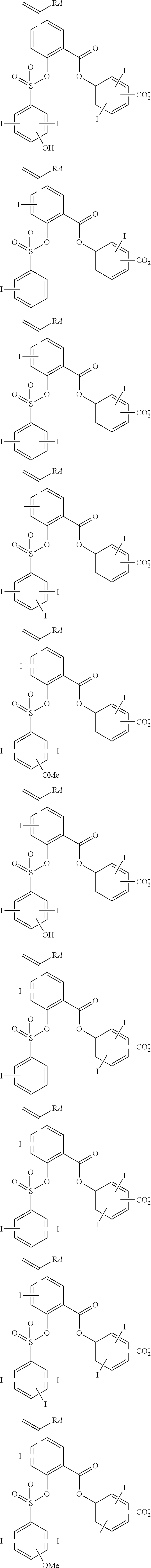

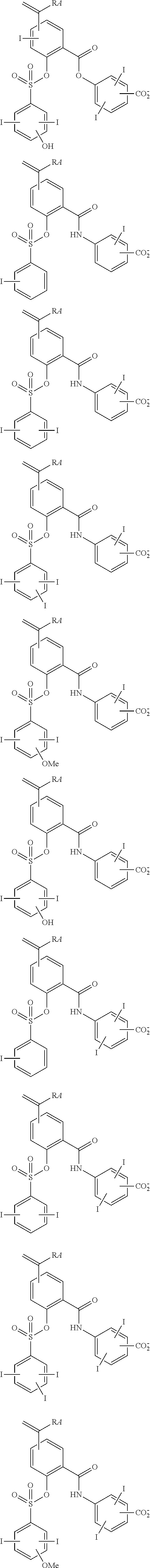

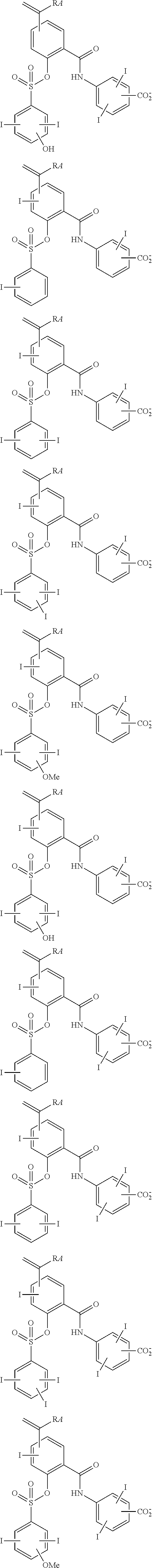

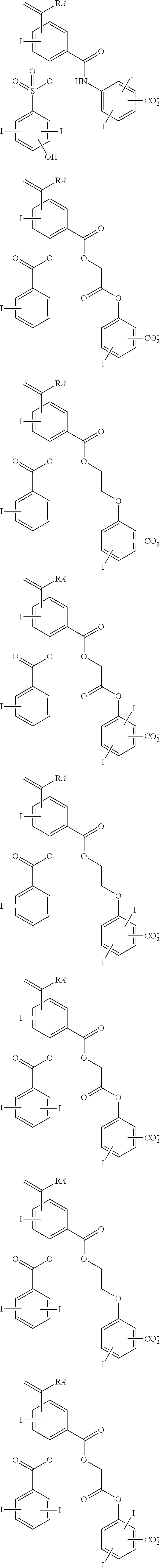

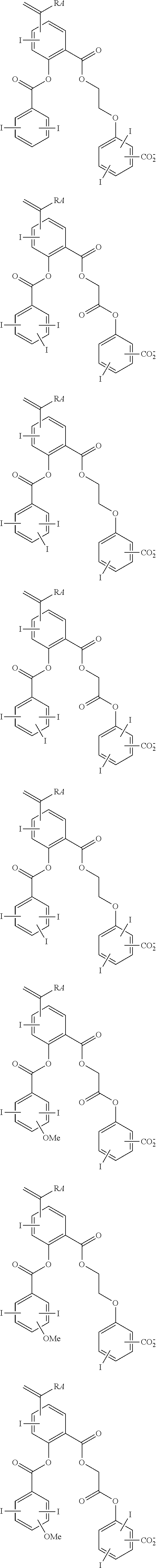

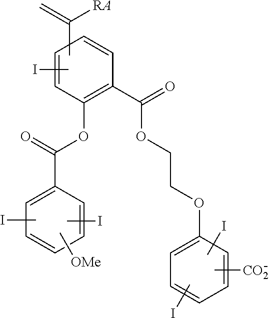

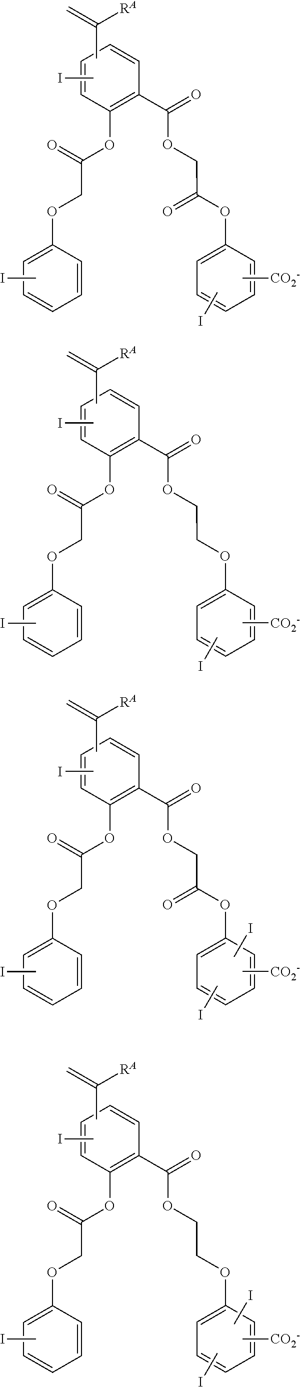

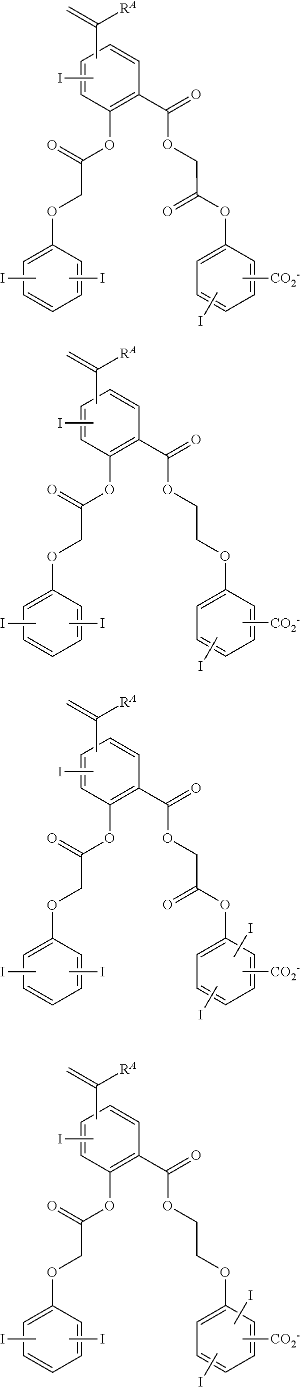

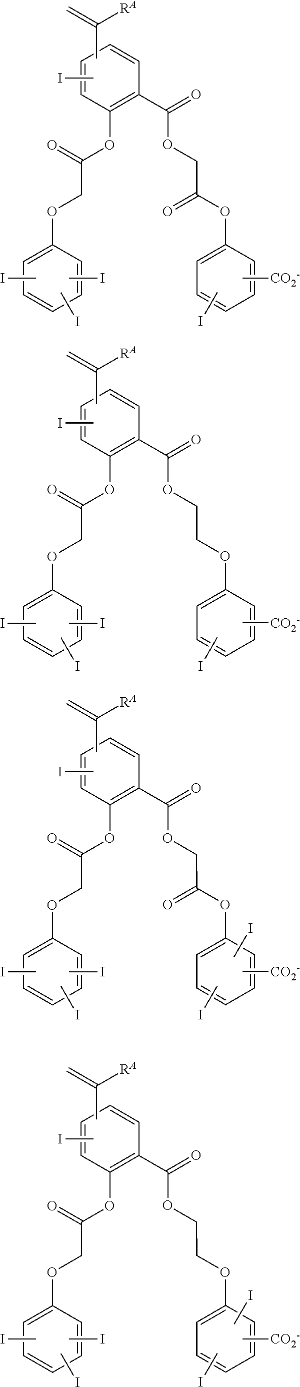

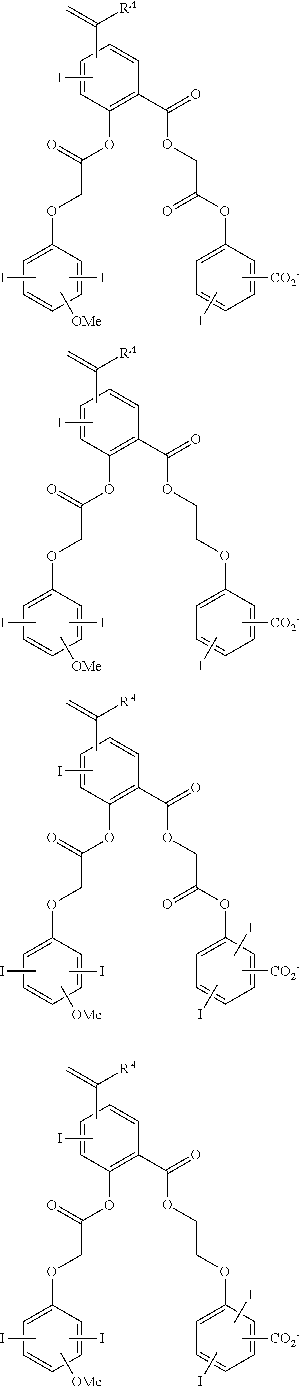

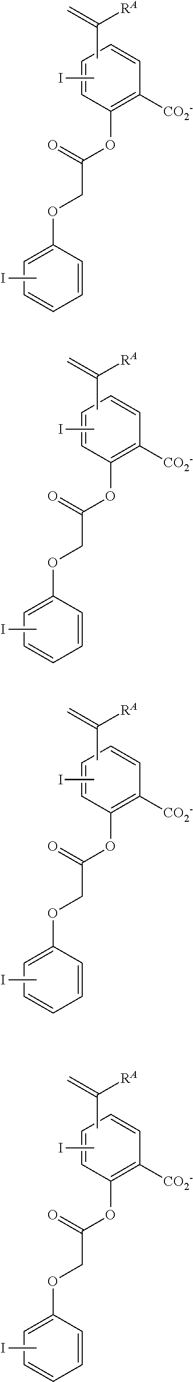

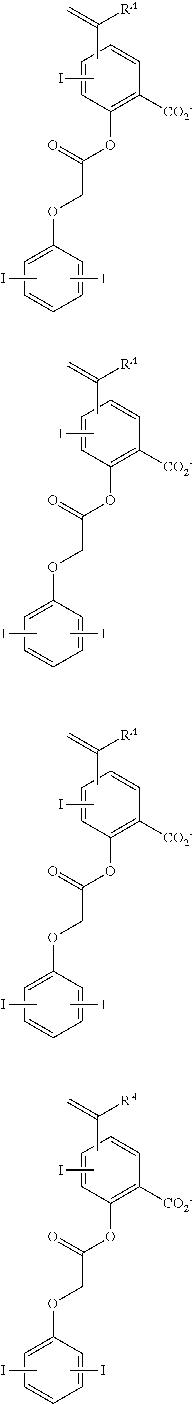

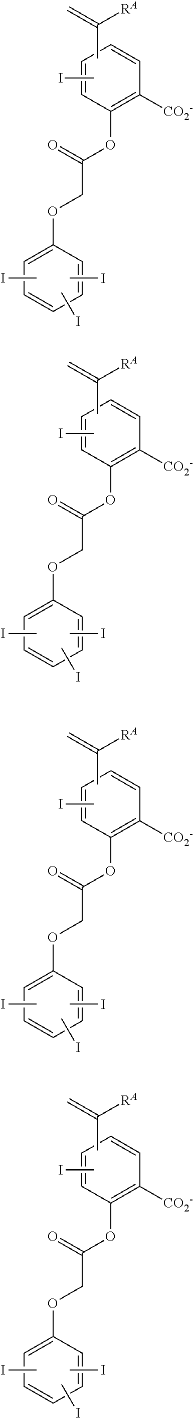

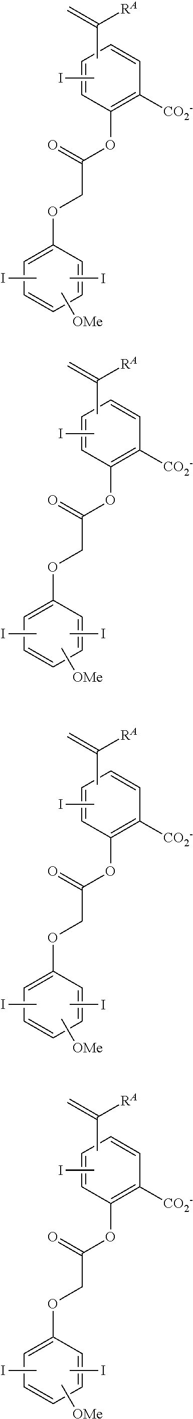

Other specific examples of the anion of the onium salt monomer represented by the general formula (a) include, but are not limited to, those shown in paragraphs [0049], [0054] to [0056], and [0062] to [0064] of JP 7433394 B.

Specific examples of the sulfonium salt monomer of the present invention include any combinations of the anions and cations shown above.

The sulfonium salt monomer of the present invention can be synthesized by a known method. Specific examples thereof include a method shown in paragraphs [0402] to [0405] of JP 7433394 B, but are not limited to this synthesis method.

### [Sulfonium Salt Quencher]

The present invention provides a sulfonium salt quencher containing the sulfonium salt monomer described above.

### [Polymer]

The polymer of the present invention contains a repeating unit derived from the sulfonium salt monomer represented by the general formula (a) (hereinafter, also referred to as repeating unit a). That is, the present invention provides a polymer containing a repeating unit derived from the sulfonium salt quencher described above.

The polymer of the present invention is a polymer-bound quencher that functions as a quencher and at the same time as a base polymer in chemically amplified resist compositions. The polymer of the present invention is structurally characterized by, for example, containing a repeating unit having a salt structure derived from the sulfonium salt quencher containing the sulfonium salt monomer of the present invention, wherein the salt structure contains a carboxylate anion having a benzene or naphthalene structure directly bound to the main chain and having an iodine atom and a sulfonium cation having a nitro group. Iodine atoms generate secondary electrons during exposure because iodine atoms exhibit very large absorption of EUV at a wavelength of 13.5 nm, and the energy of secondary electrons transfers to an acid generator to promote the decomposition, resulting in high sensitivity. The polymerizable group consisting of styrene or vinylnaphthalene structure has higher rigidity than polymerizable groups including methacrylate, and the resulting polymer has enhanced glass transition temperature (Tg). The interaction of aromatic rings (n-n stacking effect) in each base polymer molecule or between base polymer molecules is expected to cause base polymer molecules to be regularly arranged, allowing developer to develop resistance to pattern collapse even in fine patterning. Also in the etching process after fine patterning, superior etching resistance develops by virtue of the inclusion of aromatic rings directly bound to the main chain. The nitro group is a strong electron-withdrawing group, and the substitution of any aromatic ring of the triarylsulfonium cation therewith reduces the LUMO in the frontier molecular orbital theory to facilitate reception of secondary electrons, promoting the decomposition of the sulfonium cation. In addition, the nitro group, having a resonance structure in which a positive charge is present on the nitrogen atom and a negative charge is present on one oxygen atom, interacts with a proton of a generated acid at the negative charge on the oxygen atom, hence also expected to exert functions as an acid diffusion inhibitory group. The synergistic effect enables patterning resistant to pattern collapse with high sensitivity and improved LWR and CDU in combination, and thus the polymer of the present invention is suitable particularly as a material of chemically amplified positive-type resist compositions.

The polymer may further contain a repeating unit represented by general formula (b1) shown below (hereinafter, also referred to as repeating unit b1) and/or a repeating unit represented by general formula (b2) shown below (hereinafter, also referred to as repeating unit b2). That is, the polymer preferably further contains a repeating unit or repeating units represented by either one or both of the following general formulae (b1) and (b2):

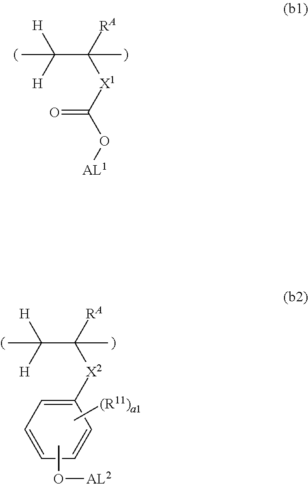

wherein R^{A} groups are each independently a hydrogen atom, a fluorine atom, a methyl group, or a trifluoromethyl group;
X¹ is a single bond, a phenylene group, a naphthylene group, *—C(═O)—O—X¹¹—, or *—C(═O)—NH—X¹¹—, wherein the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring;
X² is a single bond, *—C(═O)—O—, or *—C(═O)—NH—;
* indicates bonding to a carbon atom of a main chain;
R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom, wherein if a1 is 2 or more, R¹¹ groups are identical to or different from each other;
AL¹ and AL² are each independently an acid-unstable group; and
a1 is an integer of 0 to 4.

In the general formulae (b1) and (b2), R^{A} groups are each independently a hydrogen atom, a fluorine atom, a methyl group, or a trifluoromethyl group.

In the general formula (b1), X¹ is a single bond, a phenylene group, a naphthylene group, *—C(═O)—O—X¹¹—, or *—C(═O)—NH—X¹¹—, and the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom. X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring. * indicates bonding to a carbon atom of a main chain.

In the general formula (b2), X² is a single bond, *—C(═O)—O—, or *—C(═O)—NH—. * indicates bonding to a carbon atom of a main chain. R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. a1 is an integer of 0 to 4, and preferably 0 or 1. If a1 is 2 or more, R¹¹ groups are identical to or different from each other.

In the general formulae (b1) and (b2), AL¹ and AL² are each independently an acid-unstable group. Specific examples of the acid-unstable group include, but are not limited to, those described in JP 2013-80033 A and JP 2013-83821 A.

Specific typical examples of the acid-unstable group are those represented by the following general formulae (AL-1) to (AL-3):

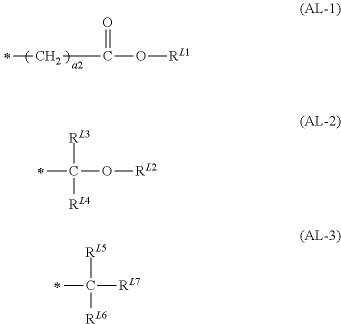

wherein * indicates bonding.

In the general formulae (AL-1) and (AL-2), R^{L1} and R^{L2} are each independently a hydrocarbyl group having 1 to 40 carbon atoms, and may contain a heteroatom such as an oxygen atom, a sulfur atom, a nitrogen atom, a fluorine atom, and an iodine atom. The hydrocarbyl group may be saturated or unsaturated, and may be linear, branched, or cyclic. The hydrocarbyl group is preferably one having 1 to 20 carbon atoms.

In the general formula (AL-1), a2 is an integer of 0 to 10, and is preferably 1, 2, 3, 4, or 5.

In the general formula (AL-2), R^{L3} and R^{L4} are each independently a hydrogen atom or a hydrocarbyl group having 1 to 20 carbon atoms, and may contain a heteroatom such as an oxygen atom, a sulfur atom, a nitrogen atom, a fluorine atom, and an iodine atom. The hydrocarbyl group may be saturated or unsaturated, and may be linear, branched, or cyclic. Any two of R^{L2}, R^{L3}, and R^{L4} are optionally bound together to form a ring having 3 to 20 carbon atoms together with the carbon atom to which the two are bound or together with the carbon atom and oxygen atom to which the two are bound. The ring is preferably a ring having 4 to 16 carbon atoms, and particularly preferably an aliphatic ring.

In the general formula (AL-3), R^{L5}, R^{L6}, and R^{L7} are each independently a hydrocarbyl group having 1 to 20 carbon atoms, and may contain a heteroatom such as an oxygen atom, a sulfur atom, a nitrogen atom, a fluorine atom, and an iodine atom. The hydrocarbyl group may be saturated or unsaturated, and may be linear, branched, or cyclic. Any two of R^{L5}, R^{L6}, and R^{L7} are optionally bound together to form a ring having 3 to 20 carbon atoms together with the carbon atom to which the two are bound. The ring is preferably a ring having 4 to 16 carbon atoms, and particularly preferably an aliphatic ring.

Specific examples of repeating unit b1 include, but are not limited to, those shown below. In the following formulae, R^{A} and AL¹ are as specified above:

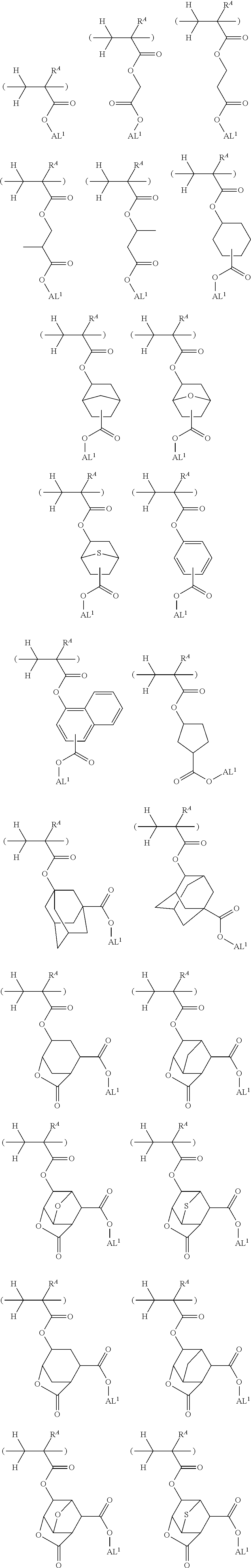

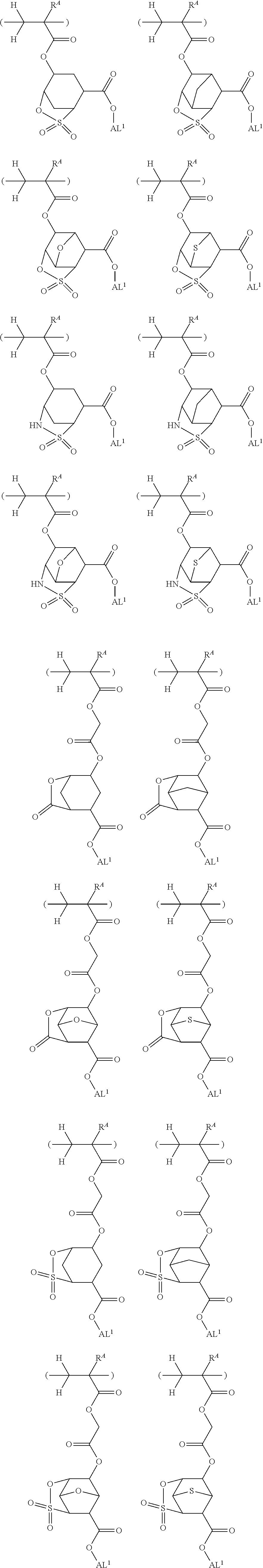

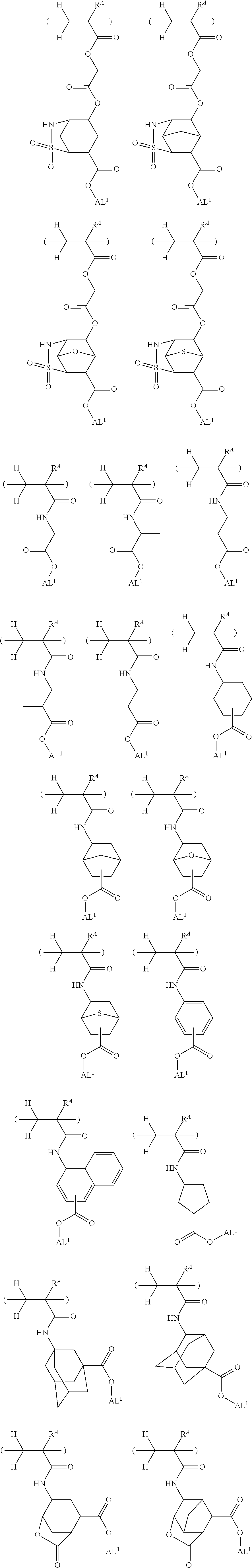

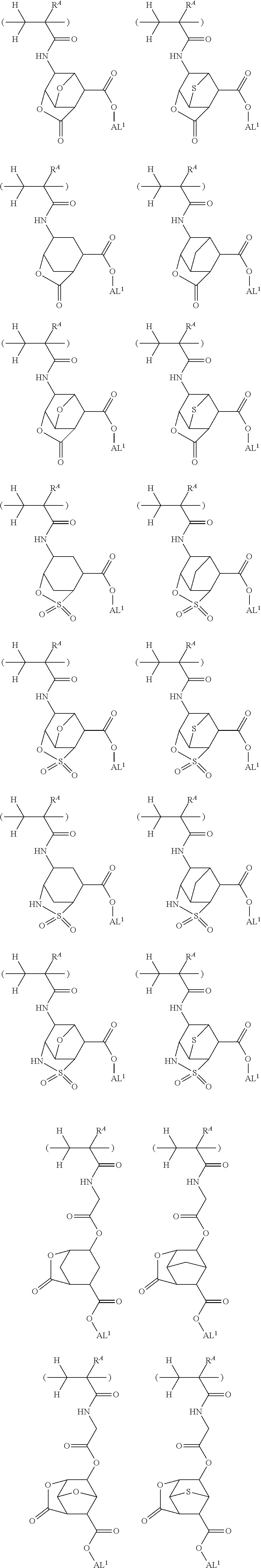

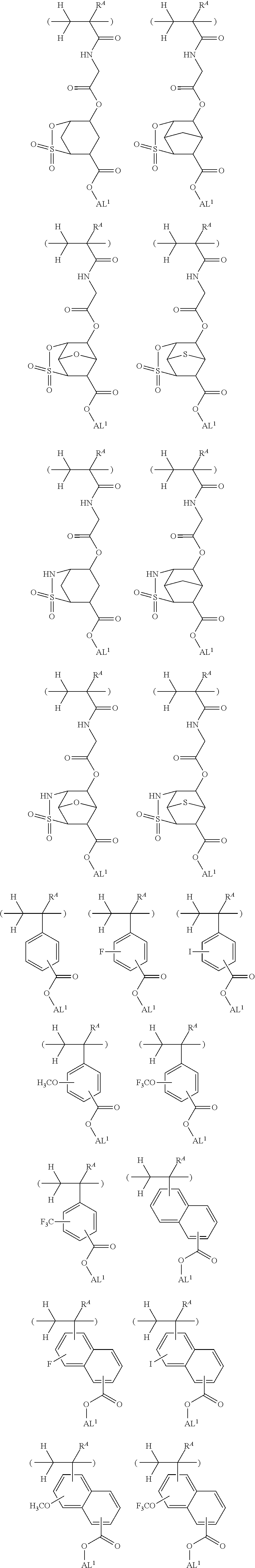

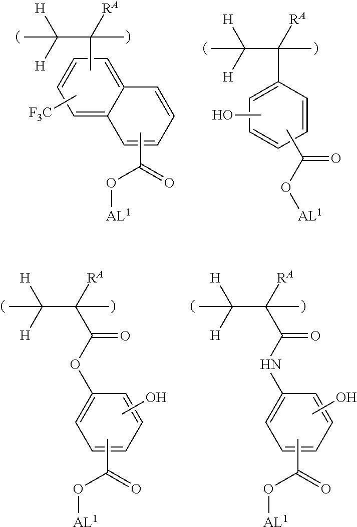

Specific examples of repeating unit b2 include, but are not limited to, those shown below. In the following formulae, R^{A} and AL² are as specified above.

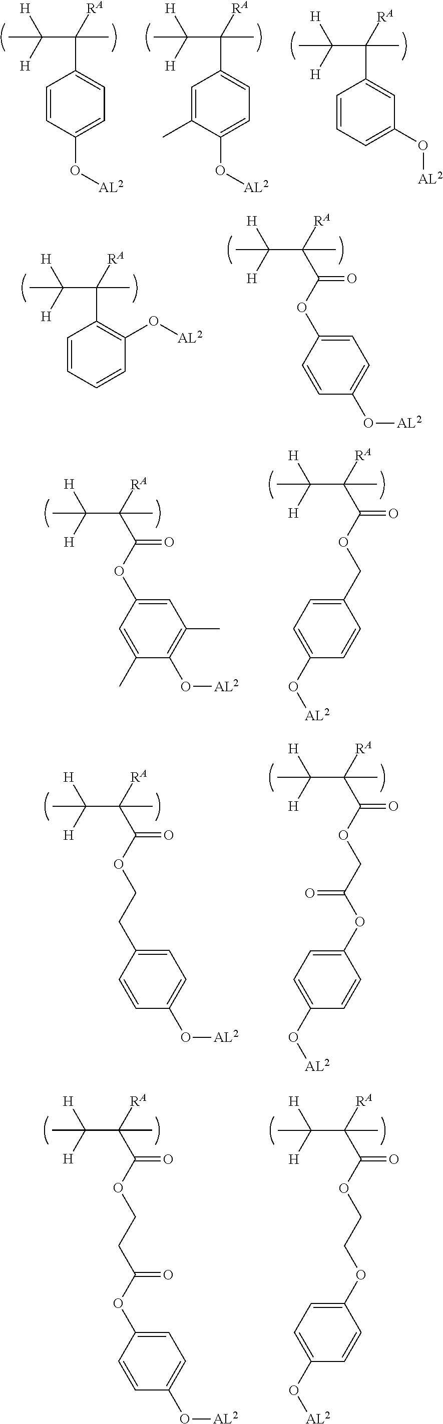

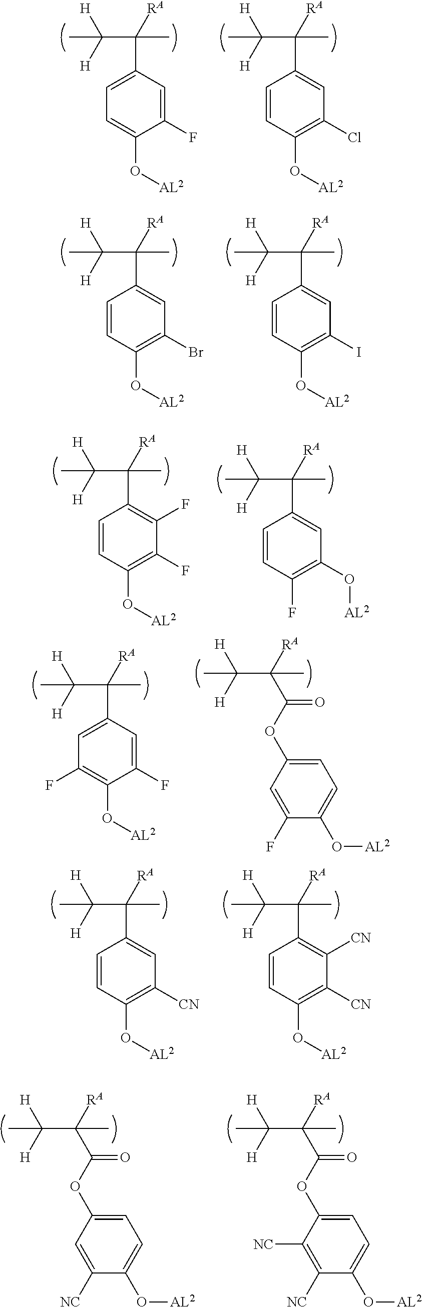

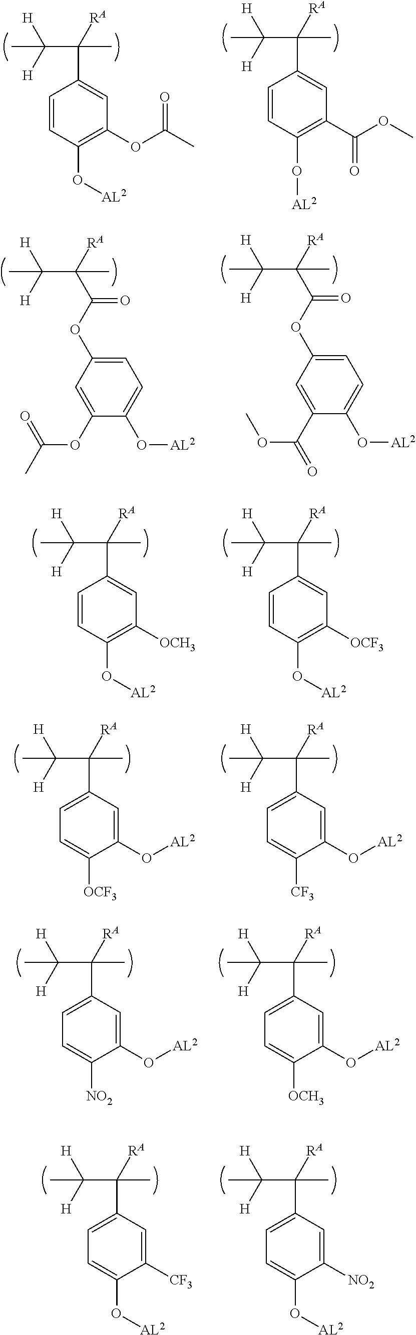

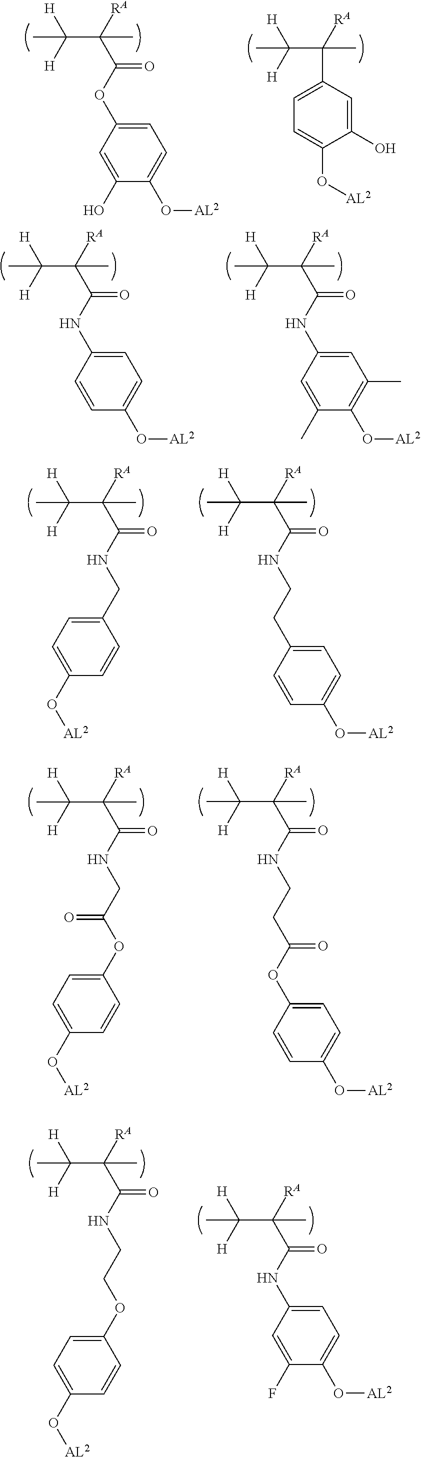

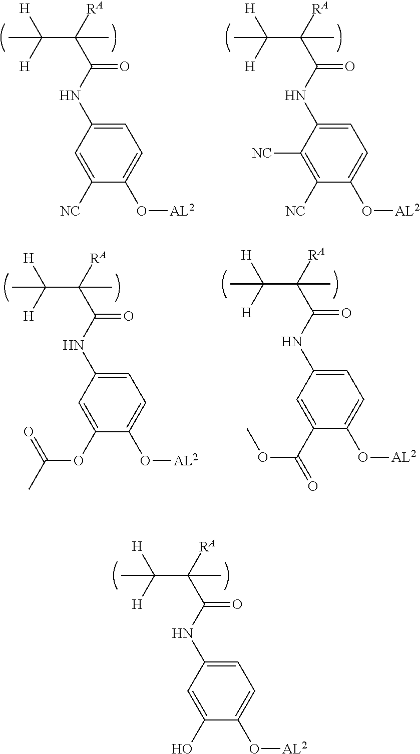

The polymer may further contain a repeating unit represented by the following general formula (b3) (hereinafter, also referred to as repeating unit b3).

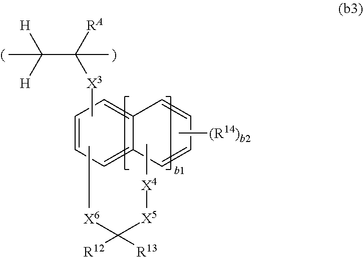

wherein b1 is 0 or 1, and b2 is an integer of 0 to 3 if b1 is 0, and an integer of 0 to 5 if b1 is 1;
R^{A} is a hydrogen atom, a fluorine atom, a methyl group, or a trifluoromethyl group;
X³ is a single bond, *—C(═O)—O—, or *—C(═O)—NH—, wherein * indicates bonding to a carbon atom of a main chain;
R¹² and R¹³ are each independently a hydrogen atom or a hydrocarbyl group having 1 to 20 carbon atoms and optionally containing a heteroatom, wherein R¹² and R¹³ are optionally bound together to form a ring together with the carbon atom to which the groups are bound;
R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A}) (R^{14B}) wherein R^{14A} and R^{14B} are each independently a hydrogen atom or a hydrocarbyl group having 1 to 6 carbon atoms, and if b2 is 2 or more, R¹⁴ groups are identical to or different from each other, and any multiple R¹⁴ groups are optionally bound together to form a ring together with the carbon atoms of the aromatic ring or aromatic rings to which the groups are bound;
X⁴ is a single bond, an aliphatic hydrocarbylene group having 1 to 4 carbon atoms, a carbonyl group, a sulfonyl group, or a group given by combining any of these; and
X⁵ and X⁶ are each independently an oxygen atom or a sulfur atom, provided that X⁴ and X⁶ are bound to adjacent carbon atoms in the aromatic ring or aromatic rings.

In the general formula (b3), b1 is 0 or 1. While a benzene ring is given if b1 is 0, and a naphthalene ring is given if b1 is 1, the case that b1 is 0 to give a benzene ring is preferred for solubility in solvents. b2 is an integer of 0 to 3 if b1 is 0, and an integer of 0 to 5 if b1 is 1. b2 is preferably 0, 1, 2, or 3, and more preferably 0, 1, or 2 for raw material procurement.

In the general formula (b3), R^{A} is a hydrogen atom, a fluorine atom, a methyl group, or a trifluoromethyl group. Among them, R^{A} is preferably a hydrogen atom or a methyl group, and further preferably a hydrogen atom.

In the general formula (b3), X³ is a single bond, *—C(═O)—O—, or *—C(═O)—NH—. * indicates bonding to a carbon atom of a main chain. Among them, X³ is preferably a single bond or *—C(═O)—O—, and more preferably a single bond.

In the general formula (b3), R¹² and R¹³ are each independently a hydrogen atom or a hydrocarbyl group having 1 to 20 carbon atoms and optionally containing a heteroatom. The hydrocarbyl group may be saturated or unsaturated, and may be linear, branched, or cyclic. Specific examples thereof include an alkyl group having 1 to 20 carbon atoms such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; a cyclic saturated hydrocarbyl group having 3 to 20 carbon atoms such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; an alkenyl group having 2 to 20 carbon atoms such as a vinyl group, an allyl group, a propenyl group, a butenyl group, and a hexenyl group; a cyclic unsaturated hydrocarbyl group having 3 to 20 carbon atoms such as a cyclohexenyl group; an aryl group having 2 to 20 carbon atoms such as a phenyl group and a naphthyl group; an aralkyl group having 7 to 20 carbon atoms such as a benzyl group, a 1-phenylethyl group, and a 2-phenylethyl group; and a group given by combining any of these. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

R¹² and R¹³ are optionally bound together to form a ring together with the carbon atom to which the groups are bound. Specific examples of the ring formed in this case include a cyclopropane ring, a cyclobutane ring, a cyclopentane ring, a cyclohexane ring, a norbornane ring, and an adamantane ring. Some or all of the hydrogen atoms in the ring may be each replaced with a group containing a heteroatom such as an oxygen atom, a sulfur atom, a nitrogen atom, and a halogen atom, some of the —CH₂— moieties in the ring may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

In the general formula (b3), R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A}) (R^{14B}). R^{14A} and R^{14B} are each independently a hydrogen atom or a hydrocarbyl group having 1 to 6 carbon atoms. The halogen atom is preferably a fluorine atom, a chlorine atom, a bromine atom, or an iodine atom, and more preferably a fluorine atom or an iodine atom. The hydrocarbyl group and the hydrocarbyl moieties of the hydrocarbyloxy group, hydrocarbyloxycarbonyl group, and hydrocarbylthio group may be each saturated or unsaturated, and may be each linear, branched, or cyclic. Specific examples thereof include those shown as examples of the hydrocarbyl group represented by R¹² and R¹³. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained. If b2 is 2 or more, R¹⁴ groups are identical to or different from each other.

If b2 is 2 or more, any multiple R¹⁴ groups are optionally bound together to form a ring together with the carbon atoms of the aromatic ring or aromatic rings to which the groups are bound. Specific examples of the ring formed in this case include a cyclopropane ring, a cyclobutane ring, a cyclopentane ring, a cyclohexane ring, a norbornane ring, and an adamantane ring. Some or all of the hydrogen atoms in the ring may be each replaced with a group containing a heteroatom such as an oxygen atom, a sulfur atom, a nitrogen atom, and a halogen atom, some of the —CH₂— moieties in the ring may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

In the general formula (b3), X⁴ is a single bond, an aliphatic hydrocarbylene group having 1 to 4 carbon atoms, a carbonyl group, a sulfonyl group, or a group given by combining any of these. Among them, X⁴ is preferably a single bond, a carbonyl group, or a sulfonyl group for raw material procurement, and more preferably a single bond or a carbonyl group in terms of the polar group produced after reaction.

In the general formula (b3), X⁵ and X⁶ are each independently an oxygen atom or a sulfur atom. It should be noted that X⁴ and X⁶ are bound to adjacent carbon atoms in the aromatic ring or aromatic rings. While X⁵ and X⁶ are identical to or different from each other, X⁵ and X⁶ are preferably each an oxygen atom for reactivity.

Specific examples of repeating unit b3 include, but are not limited to, those shown below. In the following formulae, R^{A} is as specified above, and Me is a methyl group. The bonding positions of substituents in each aromatic ring may be interchanged therein.

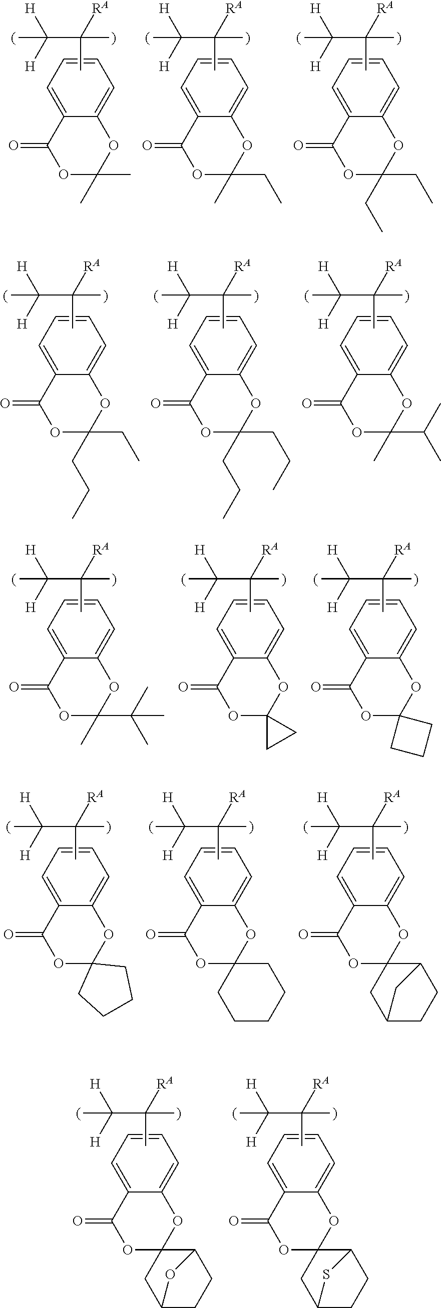

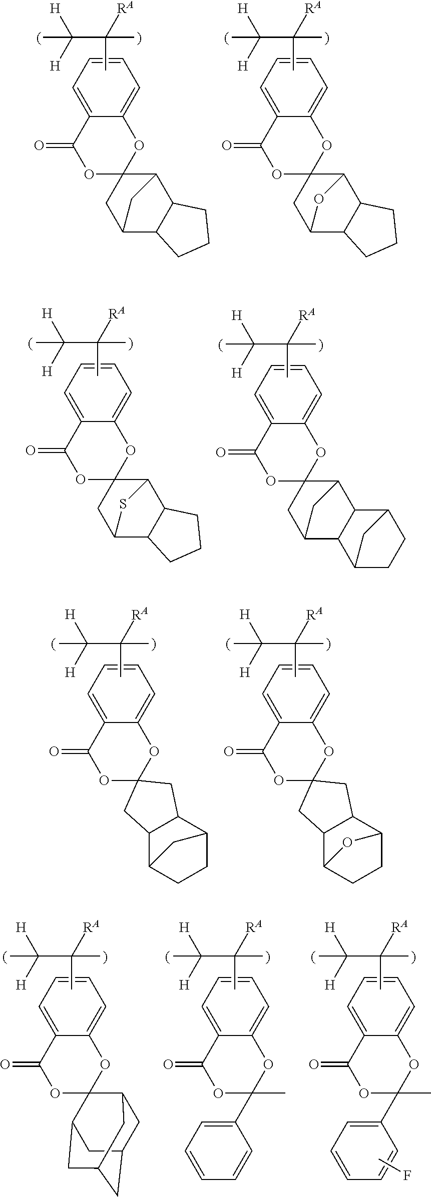

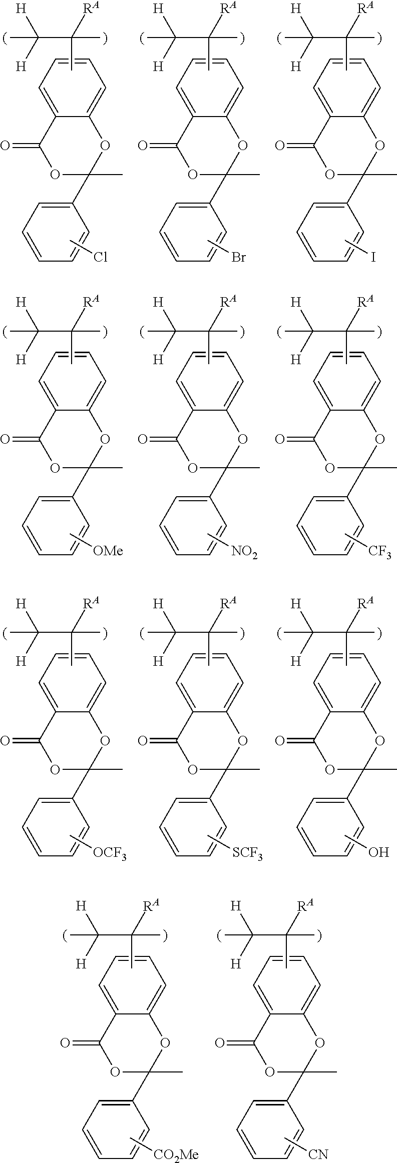

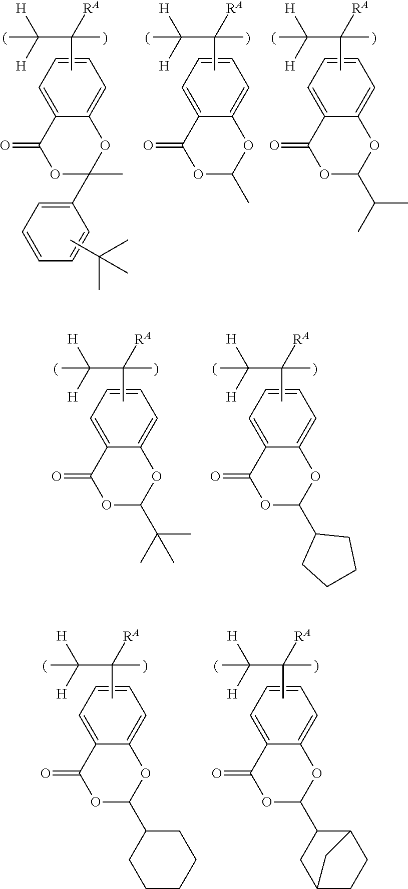

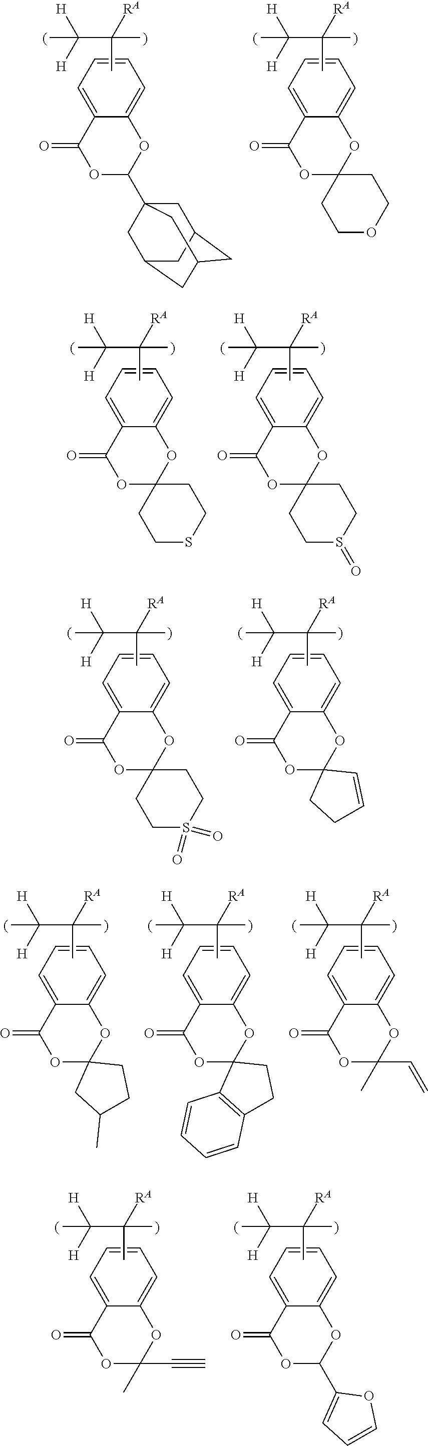

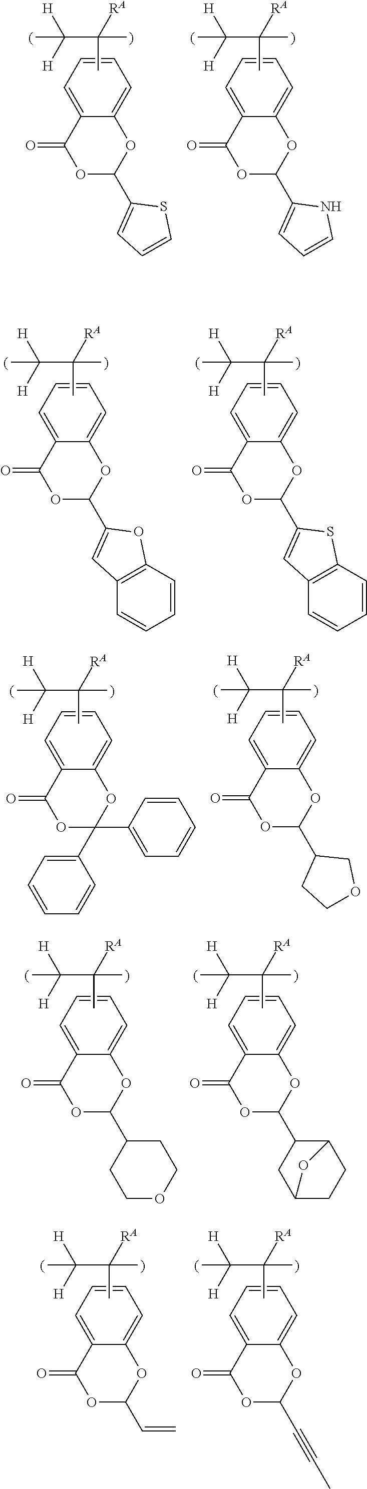

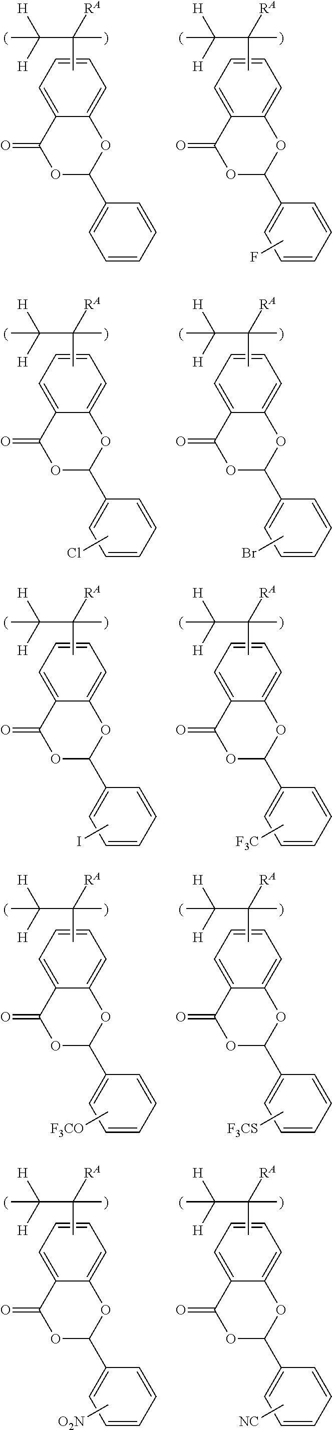

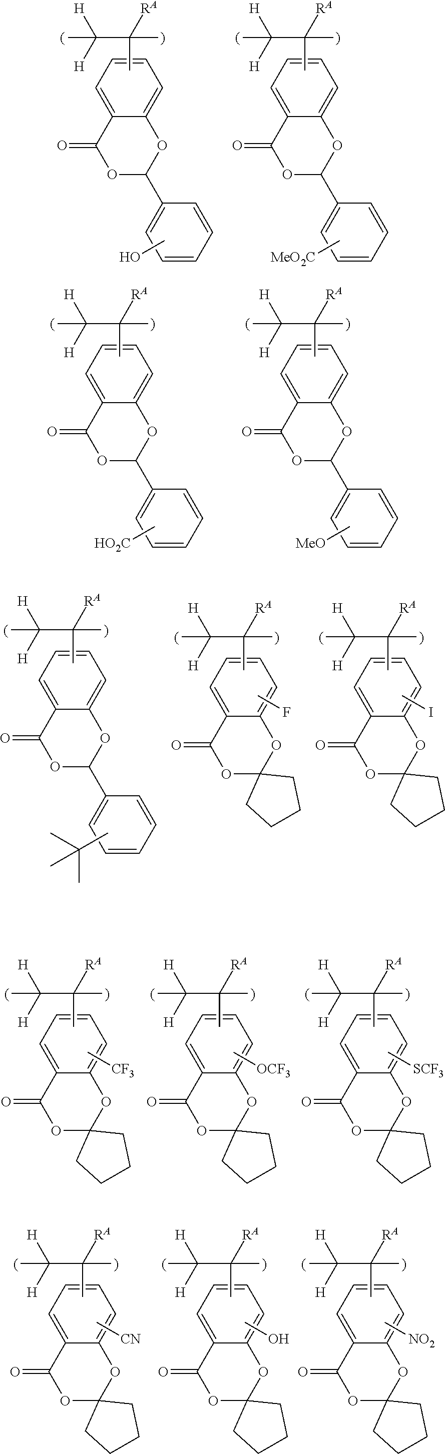

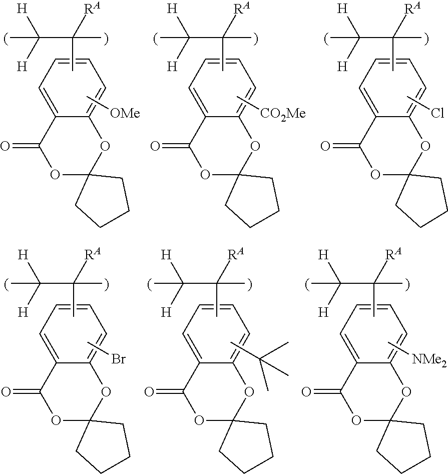

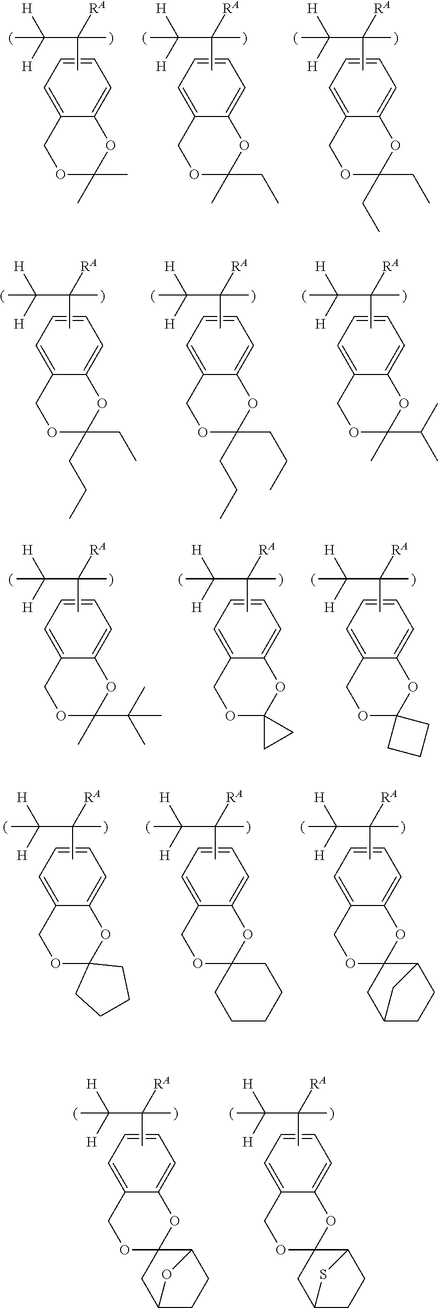

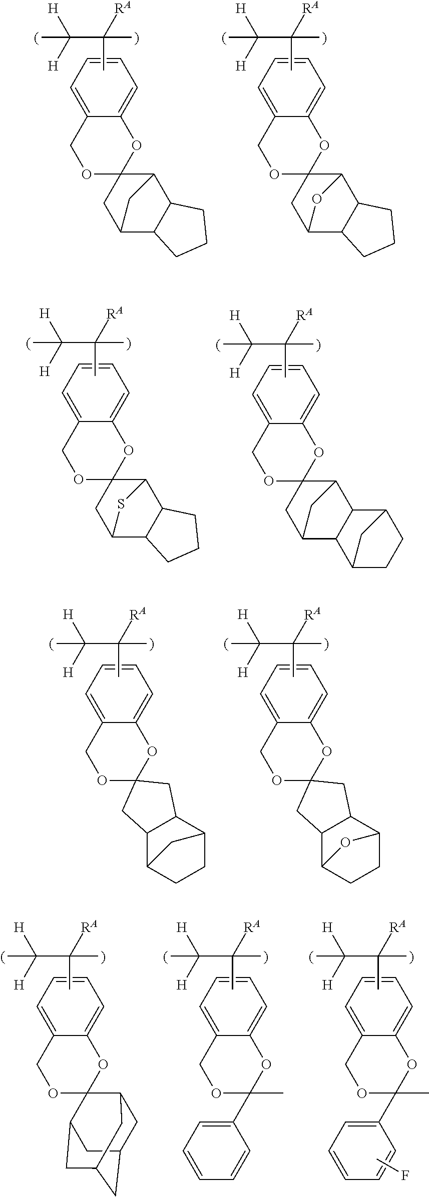

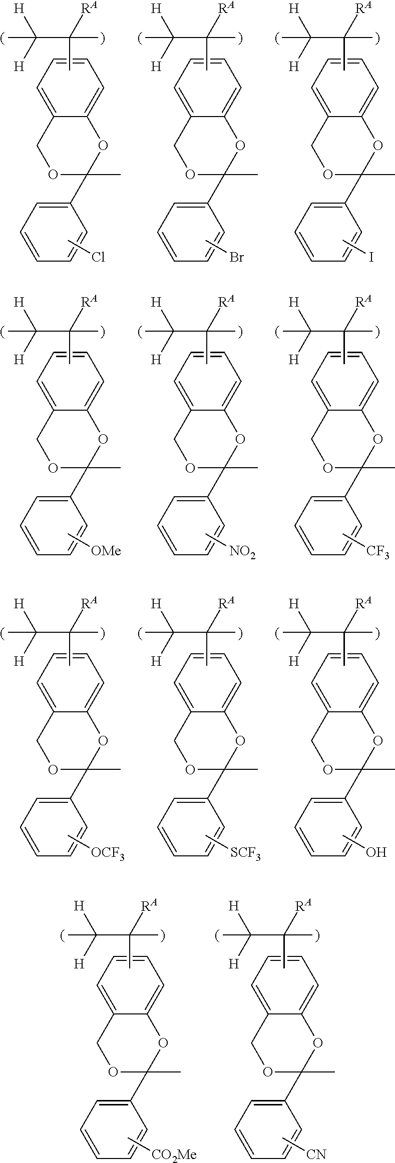

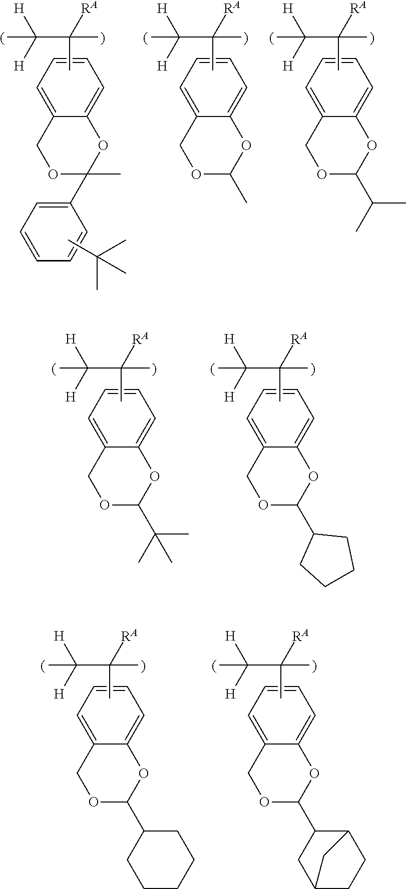

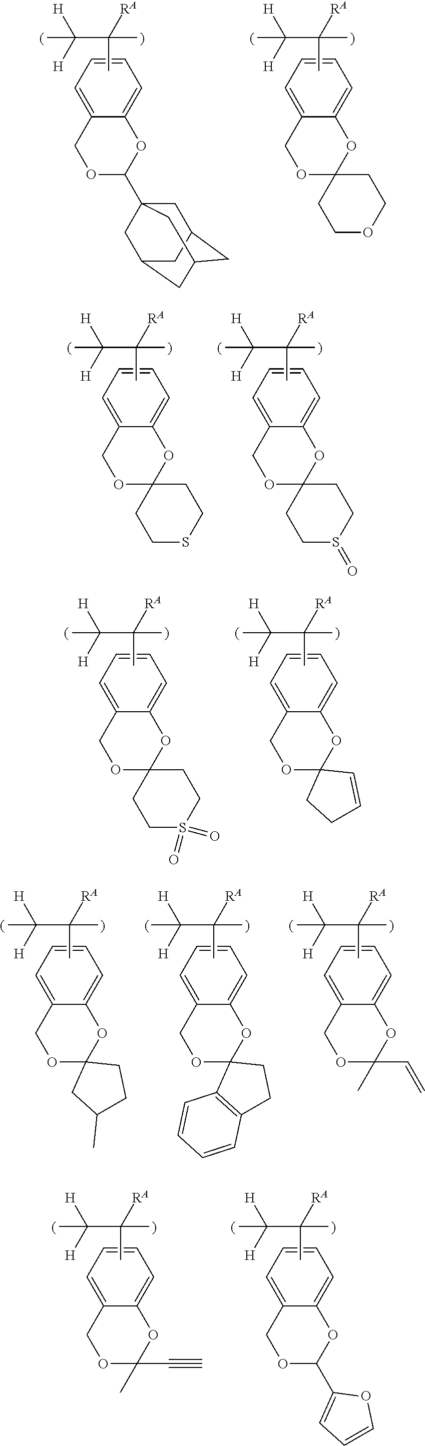

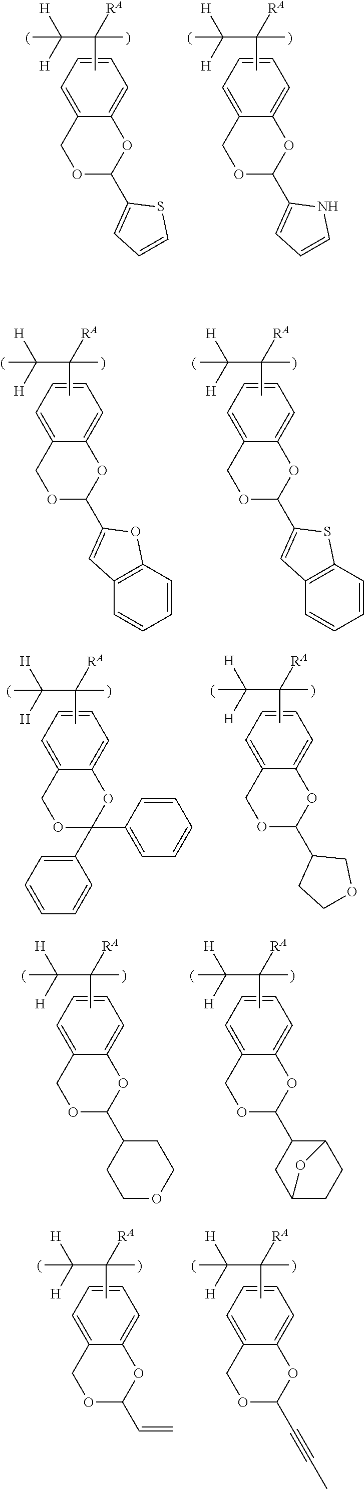

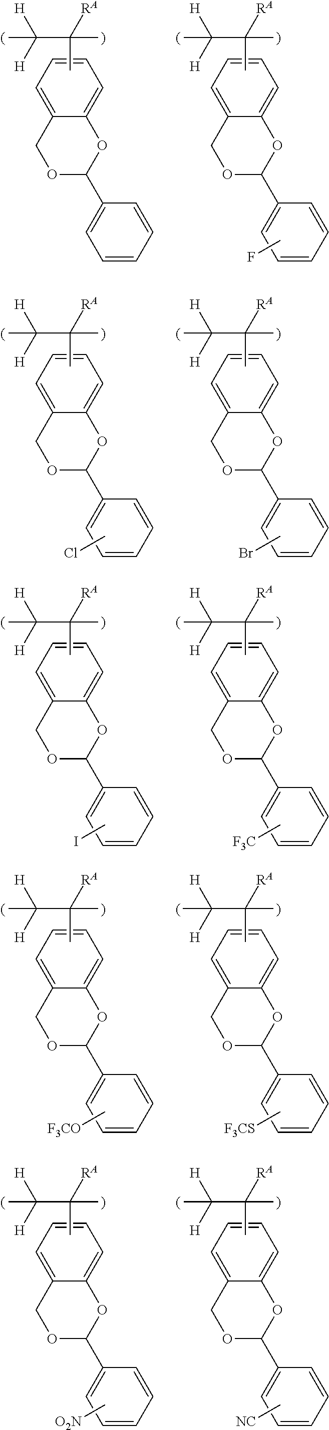

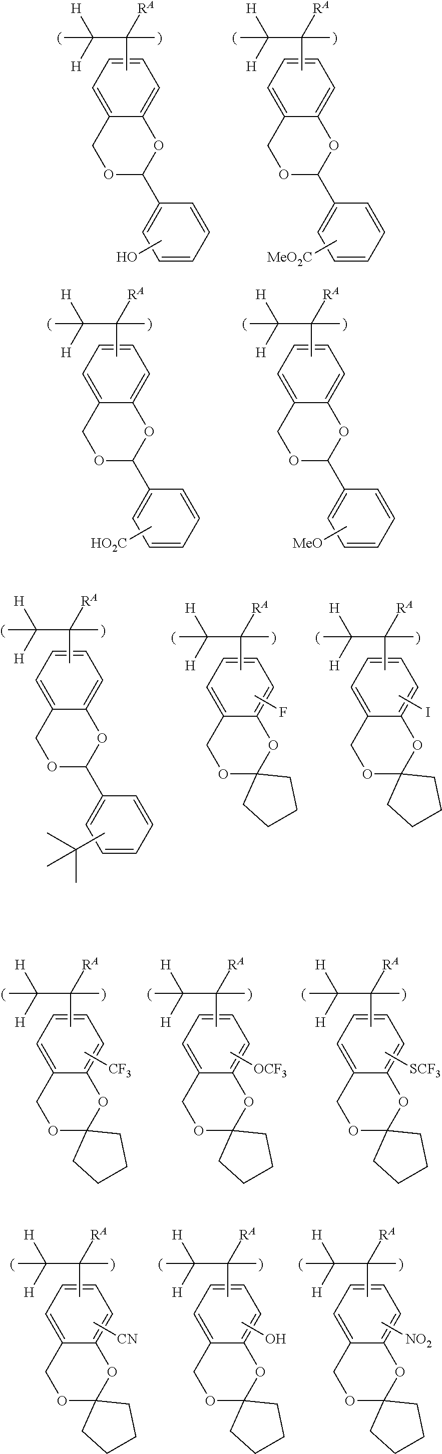

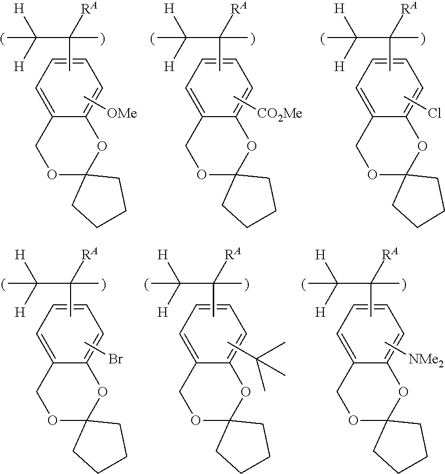

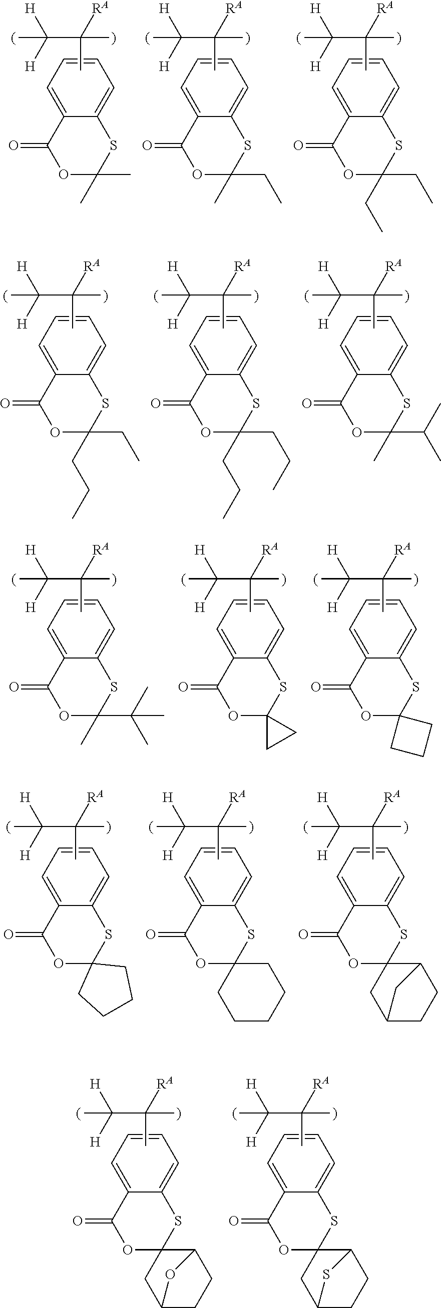

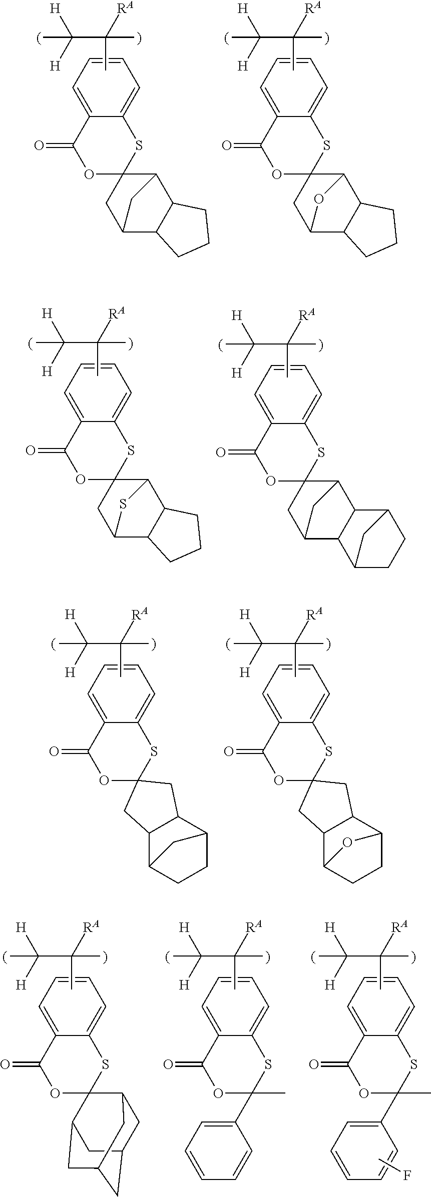

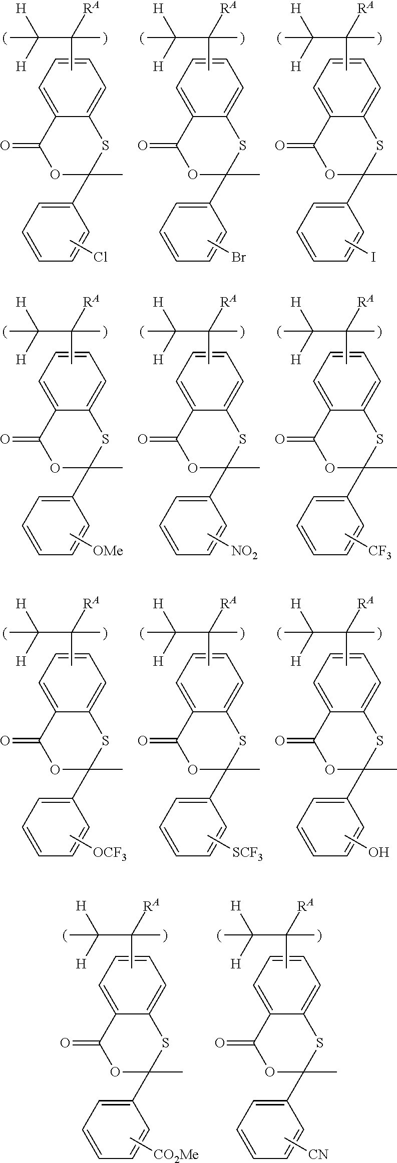

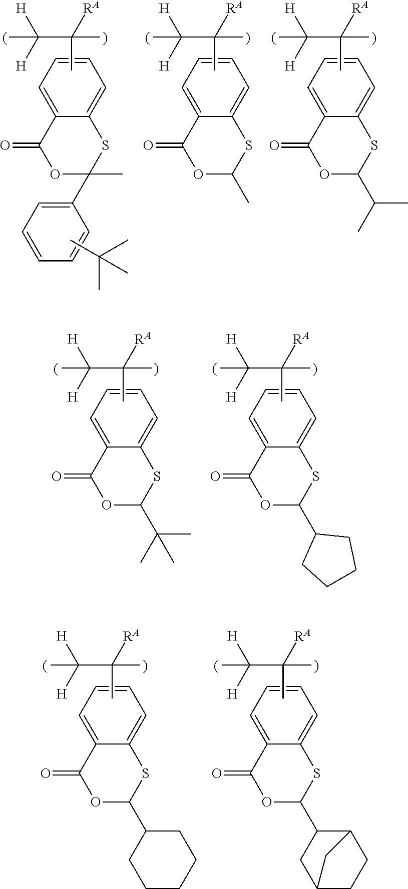

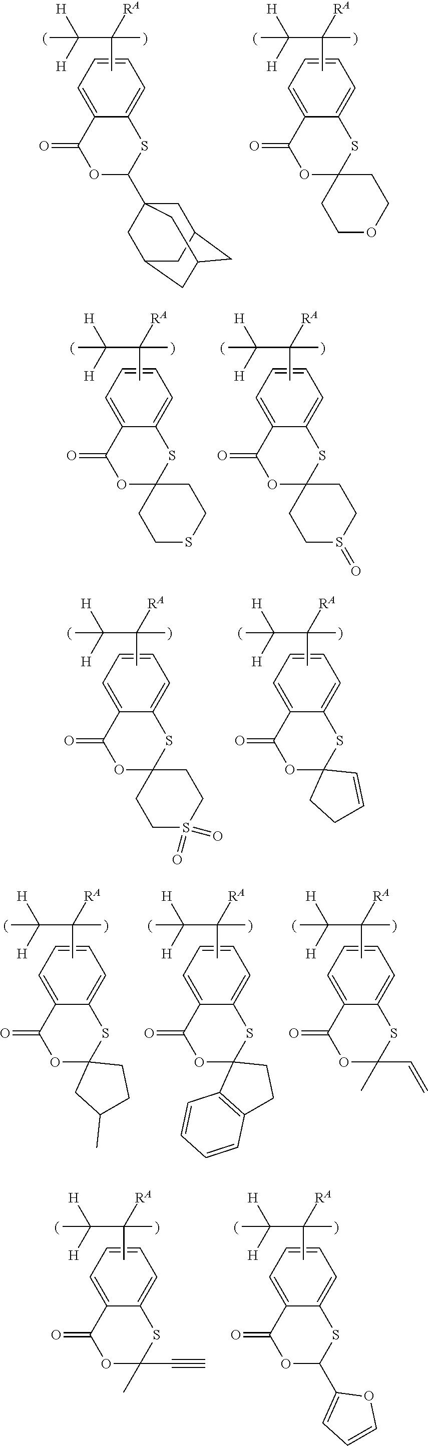

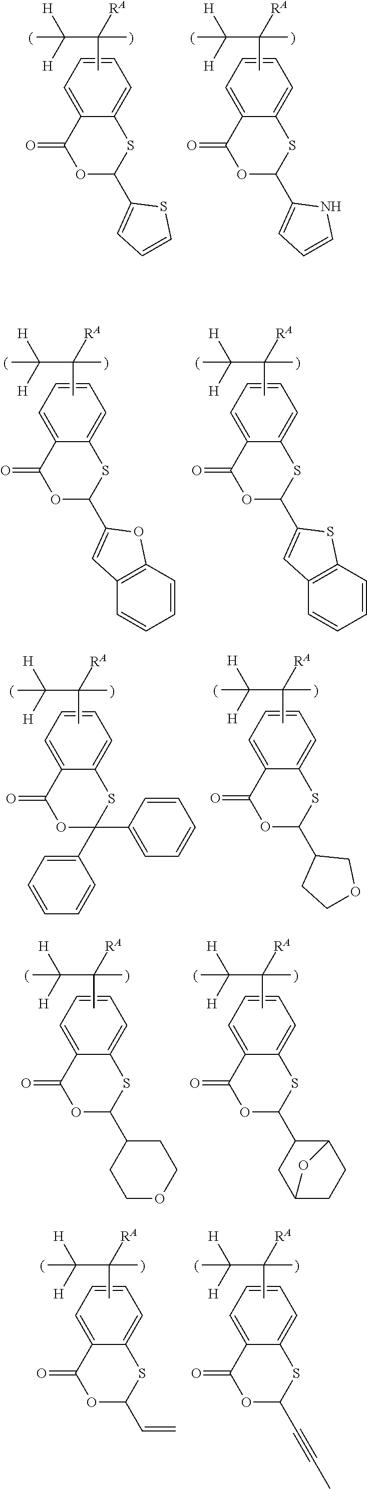

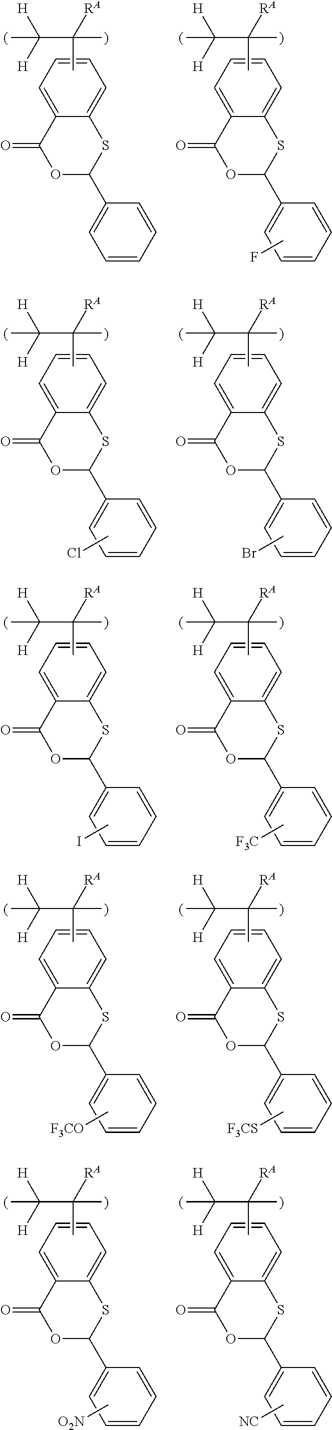

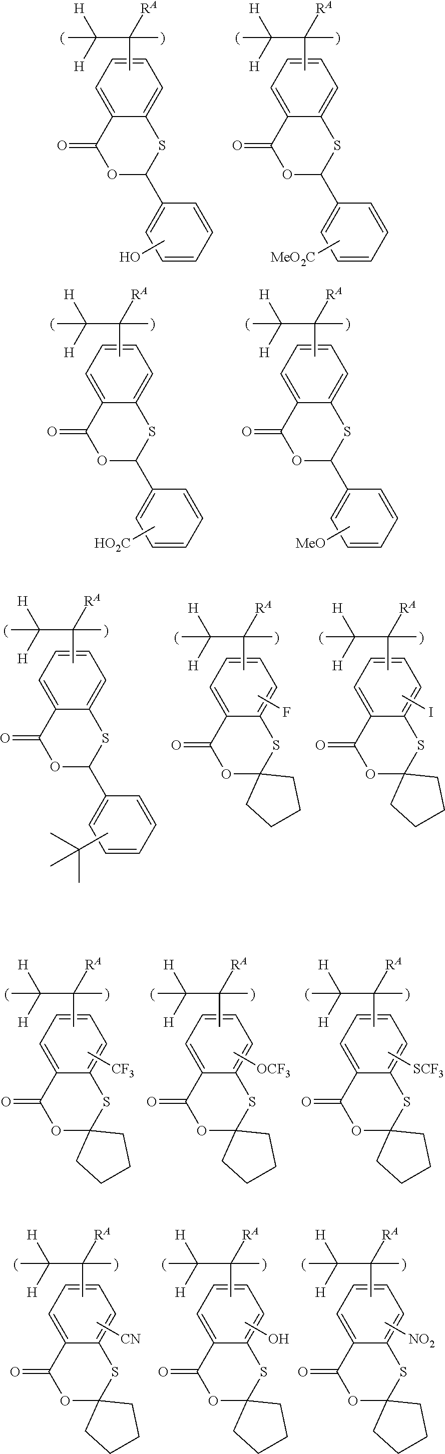

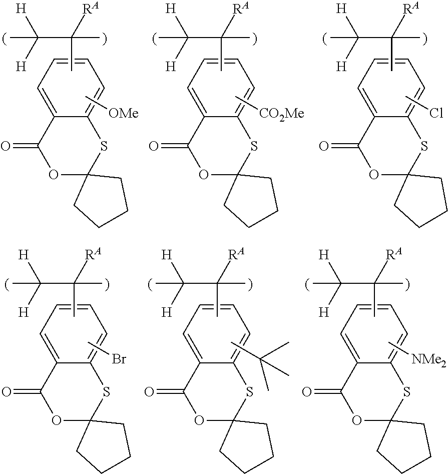

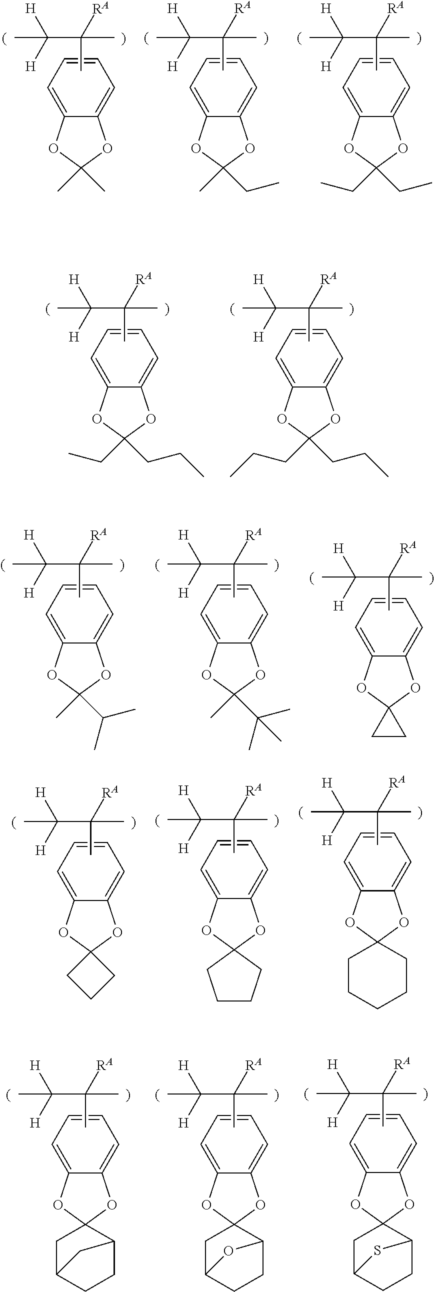

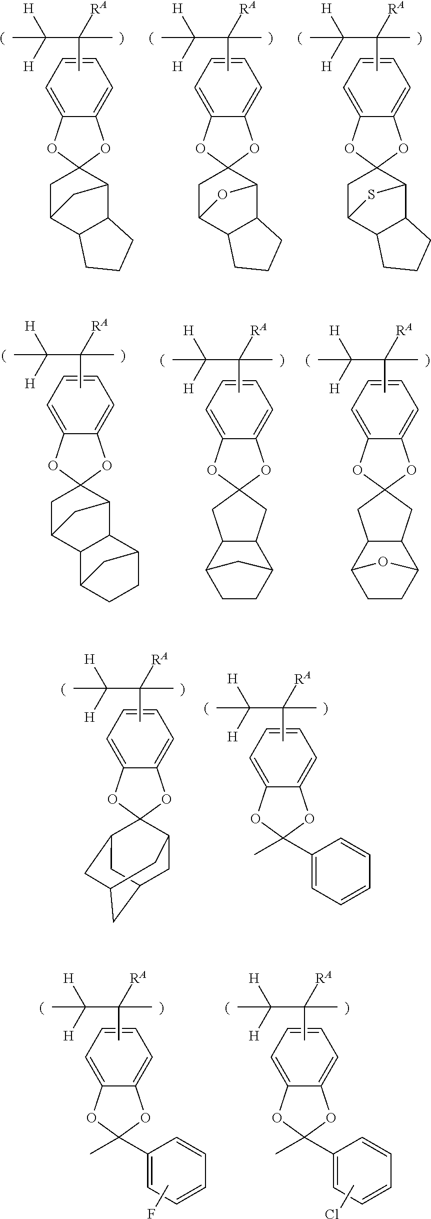

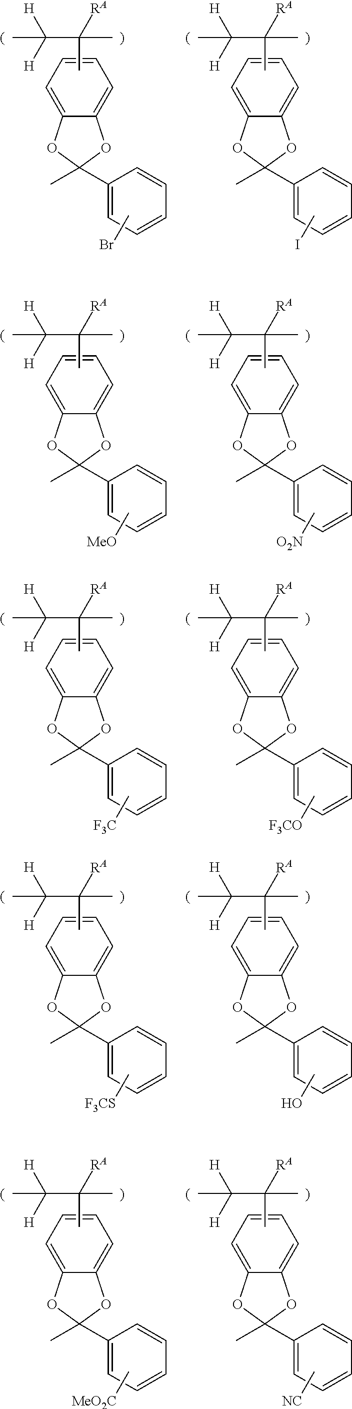

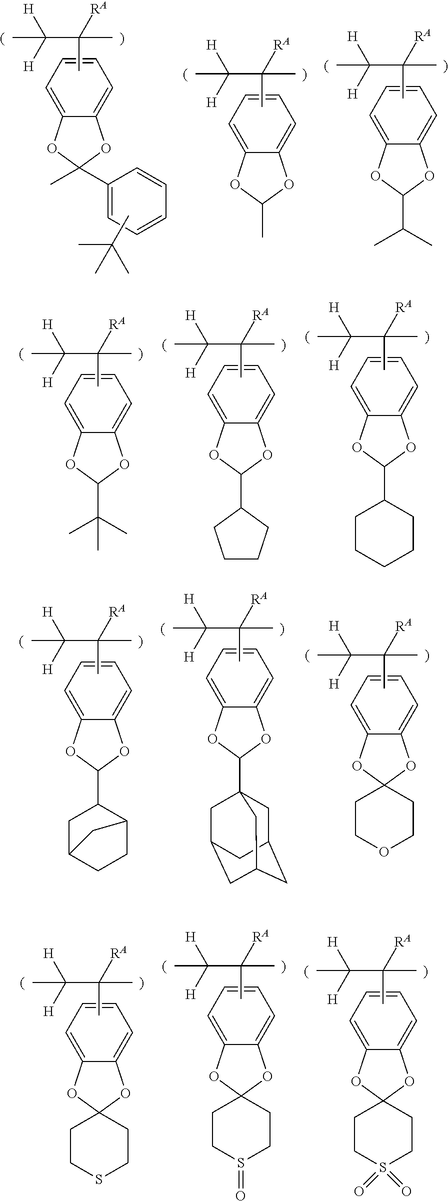

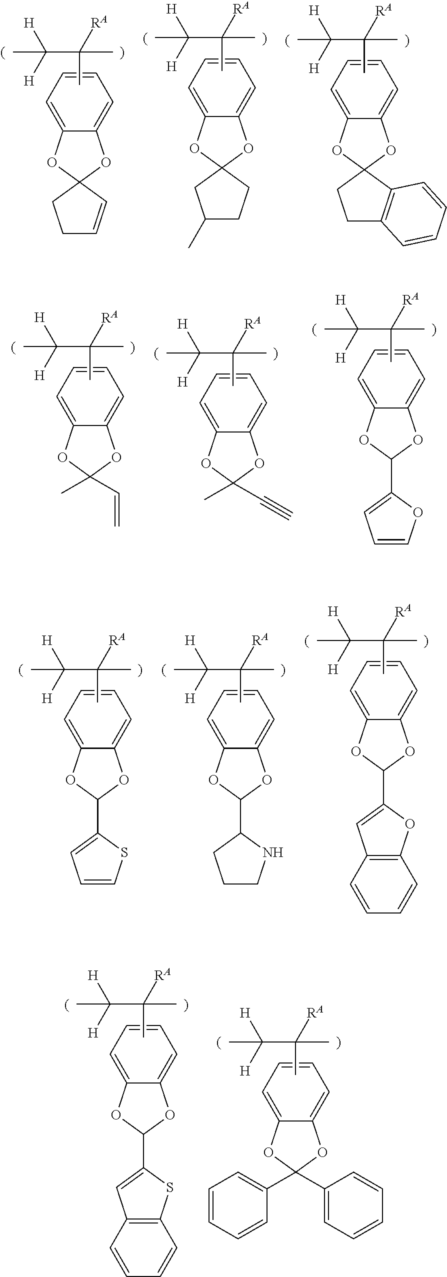

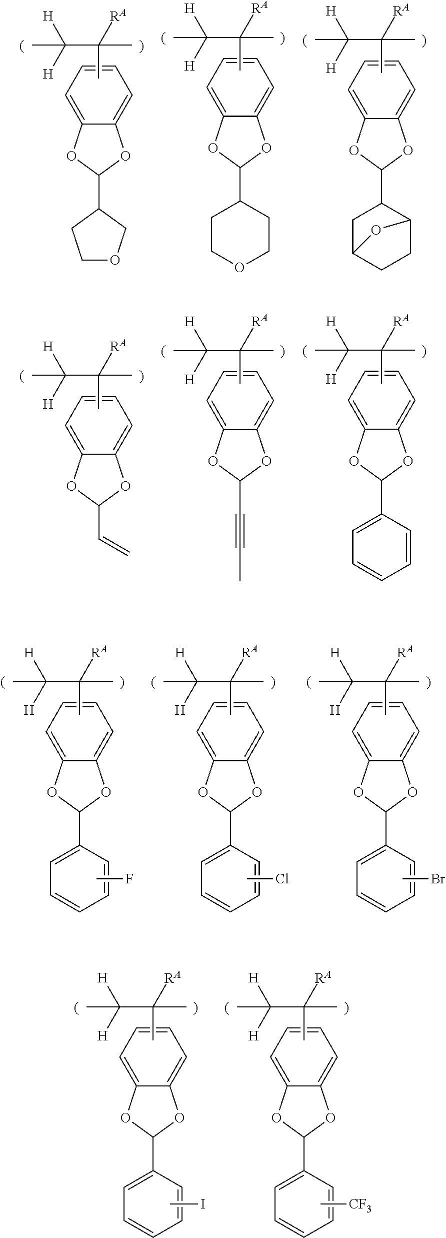

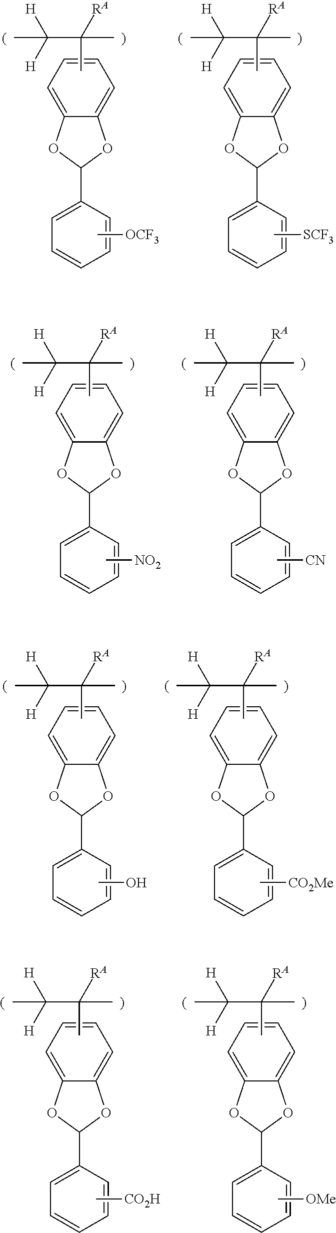

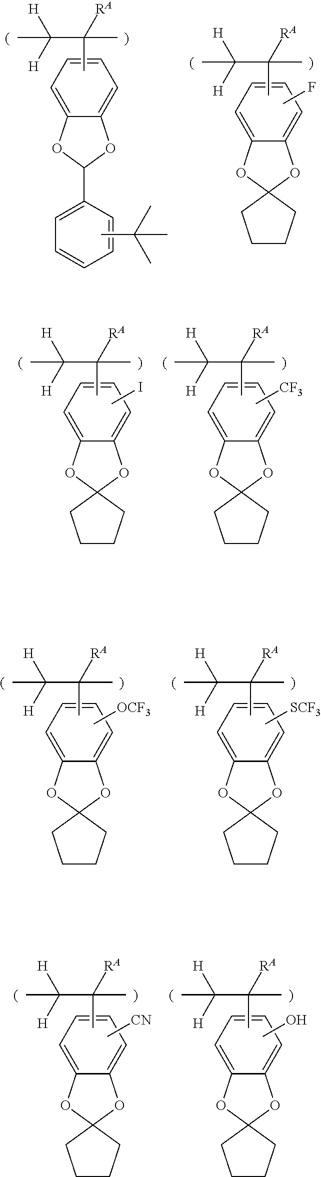

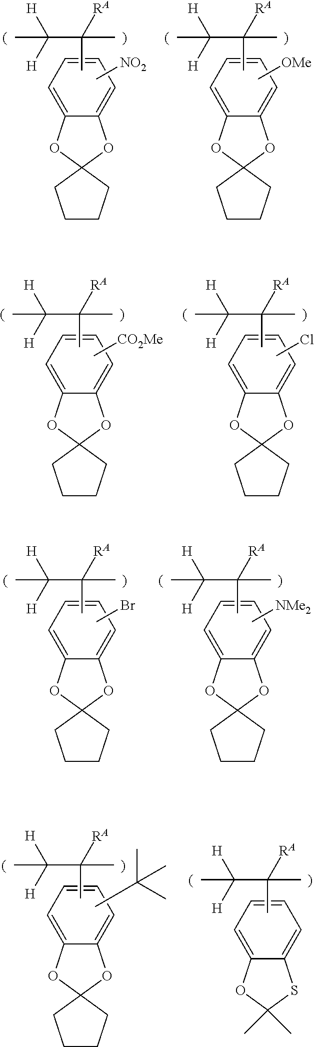

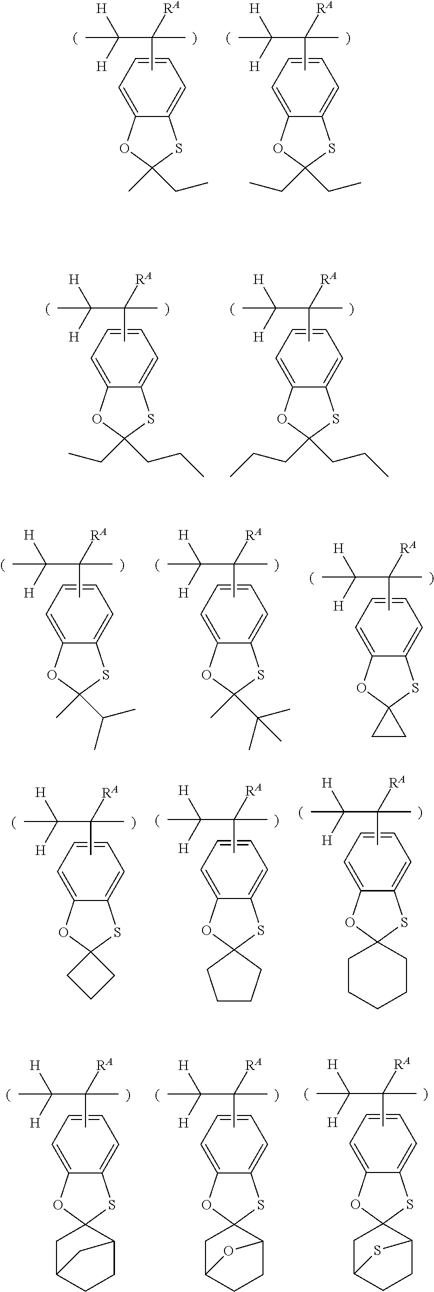

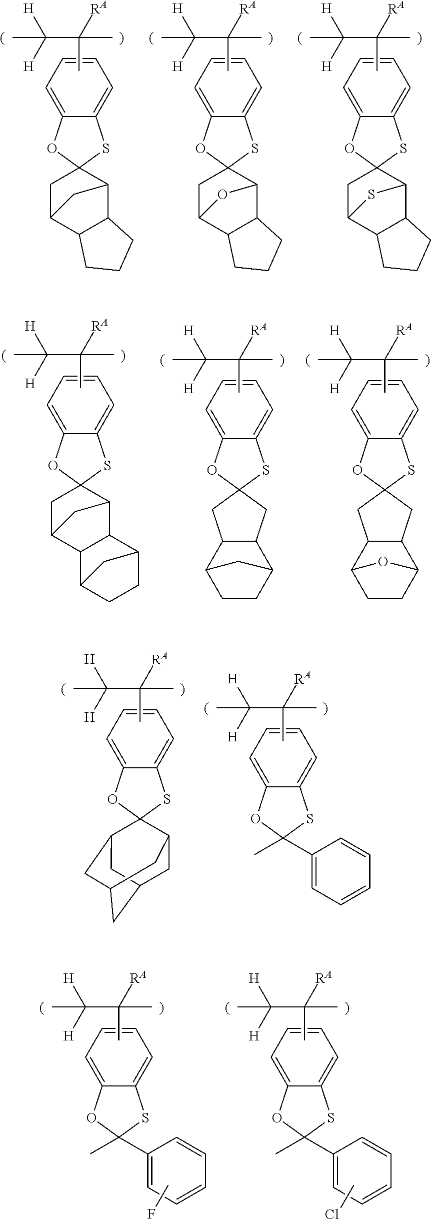

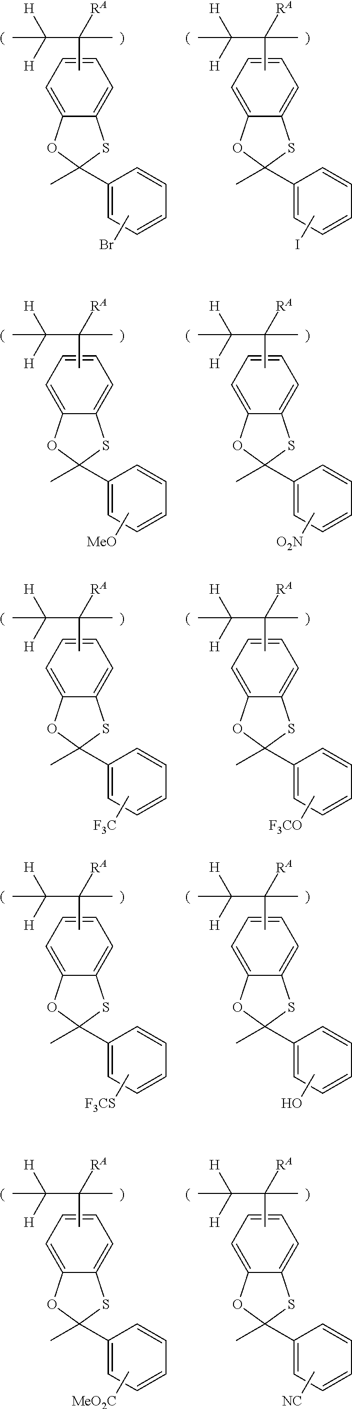

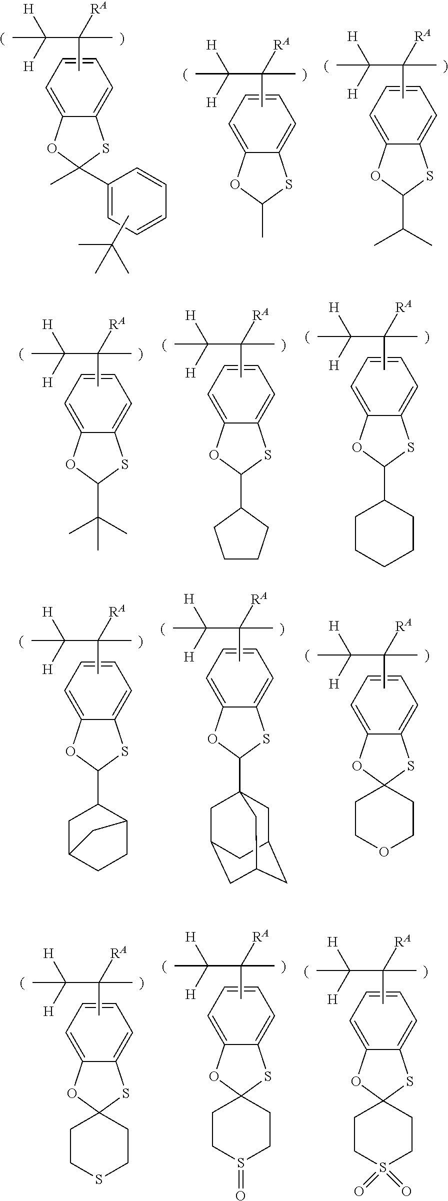

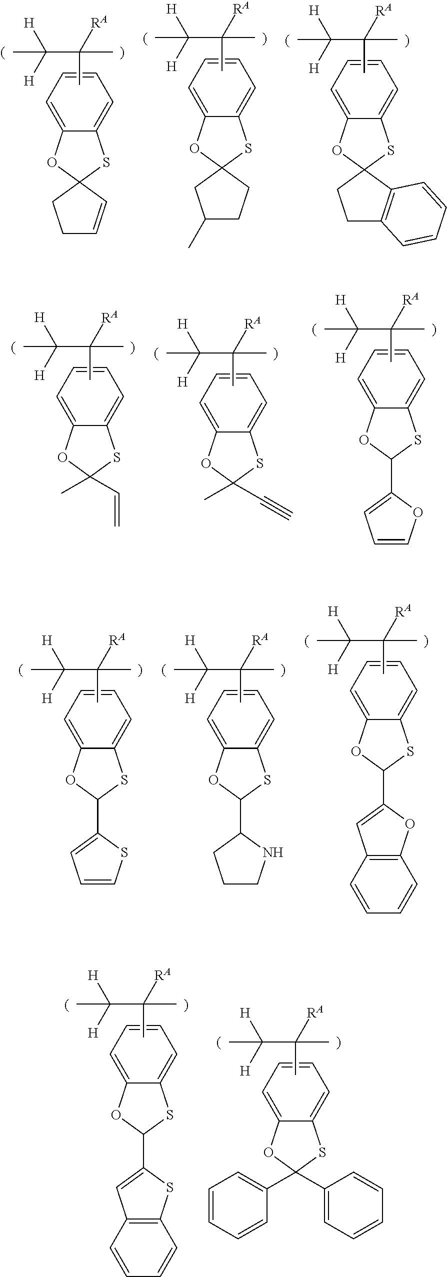

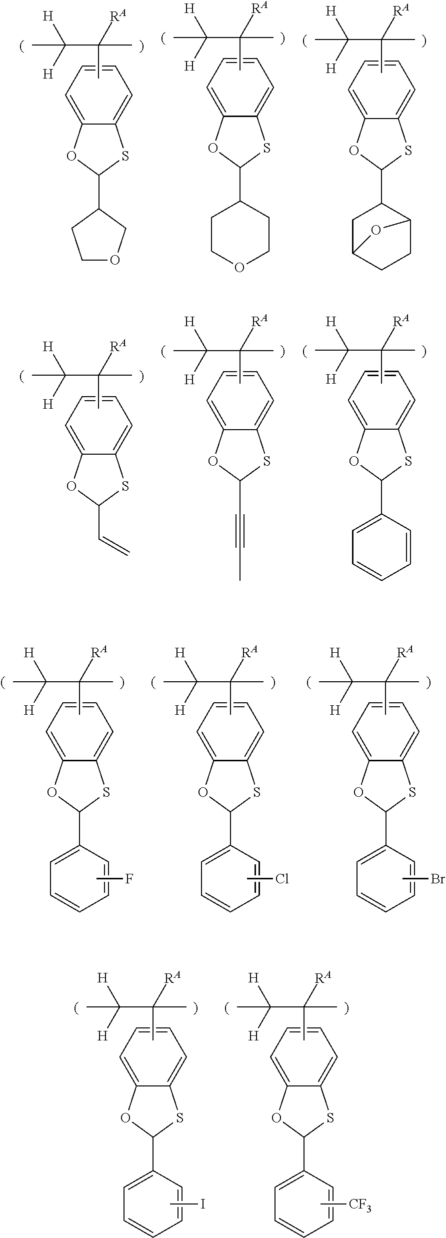

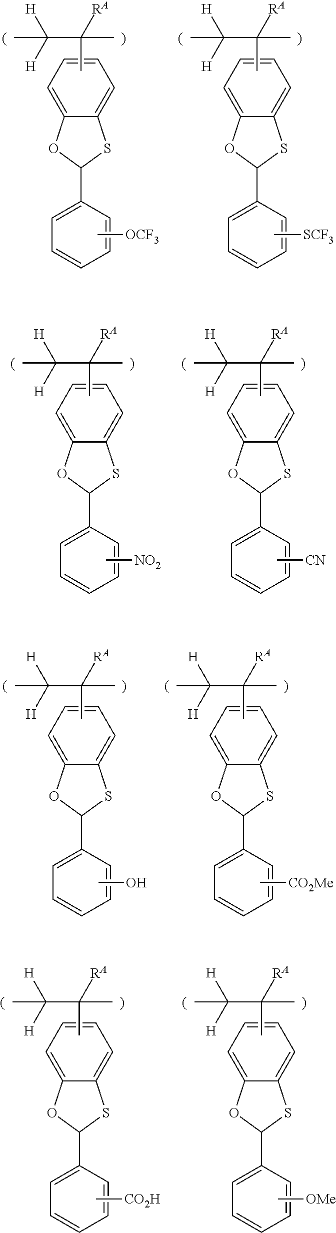

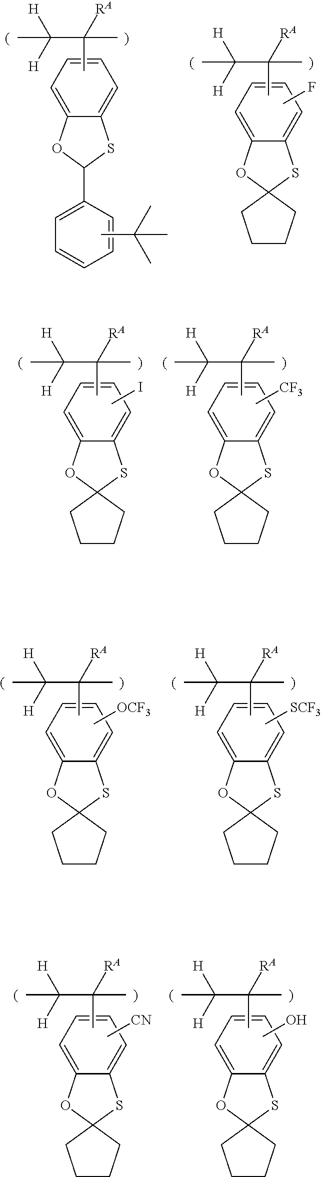

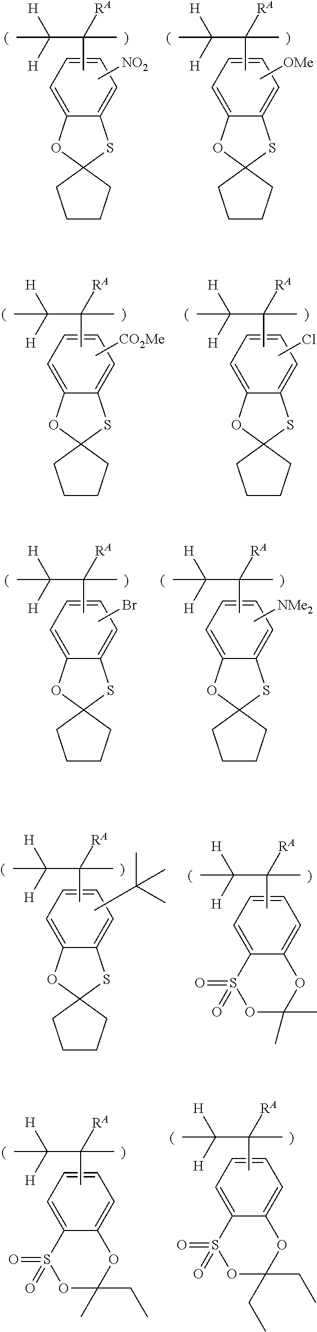

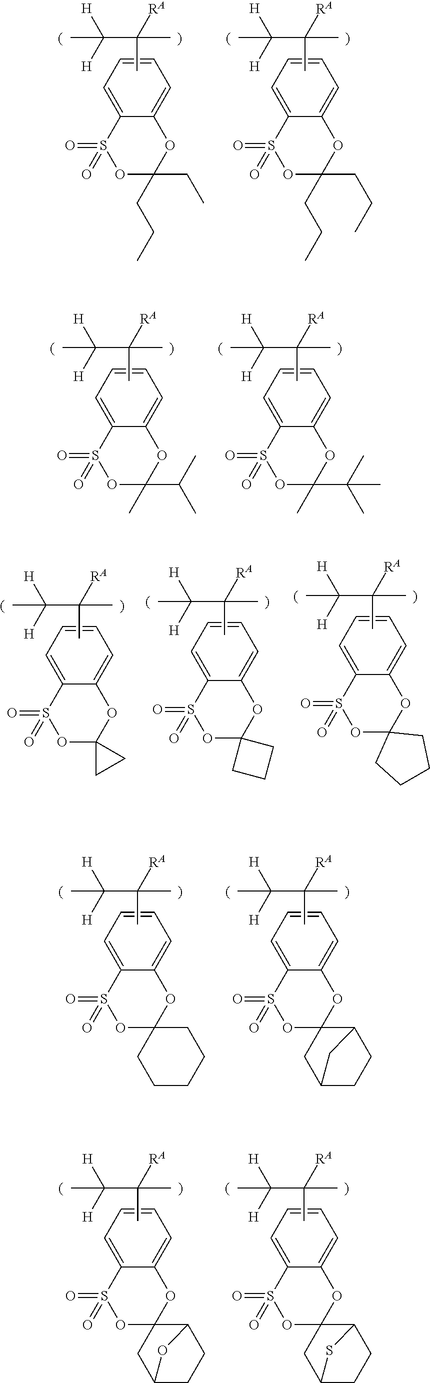

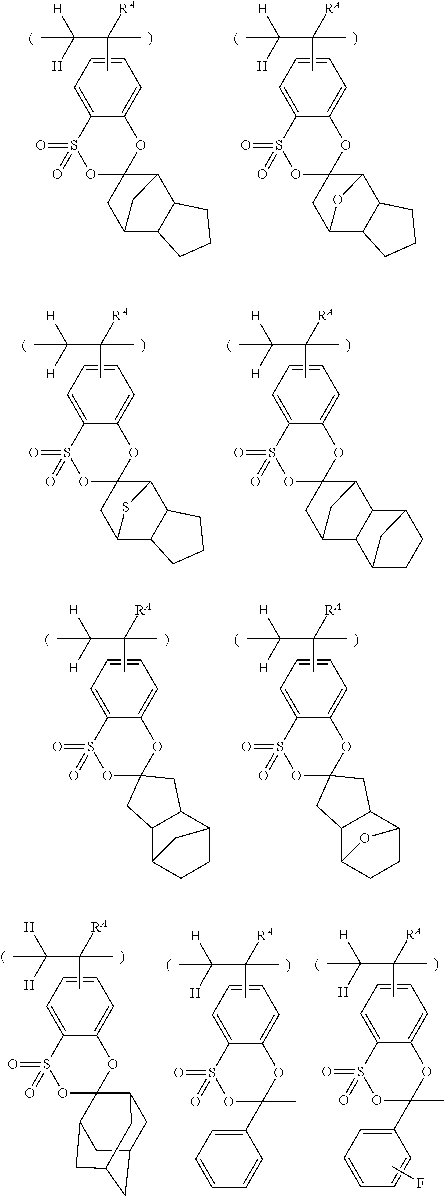

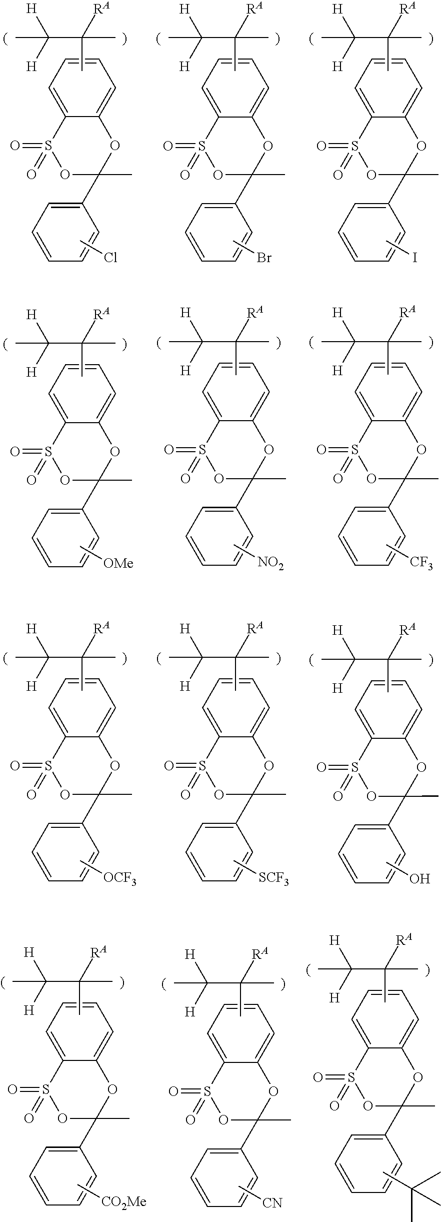

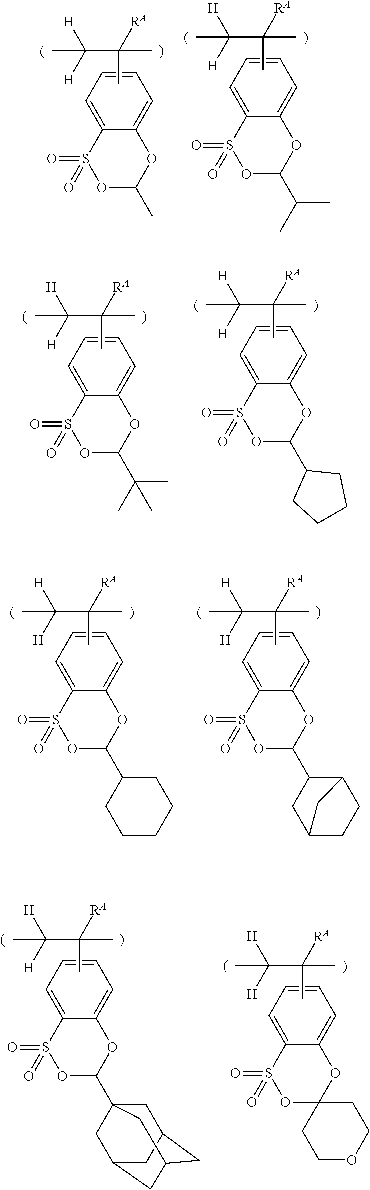

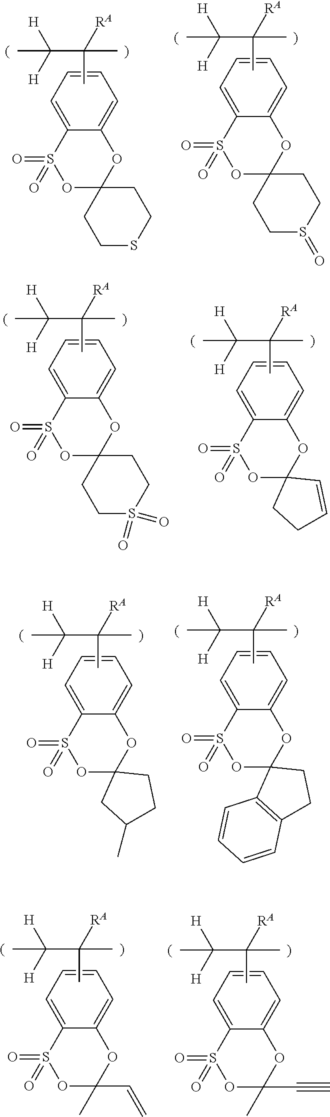

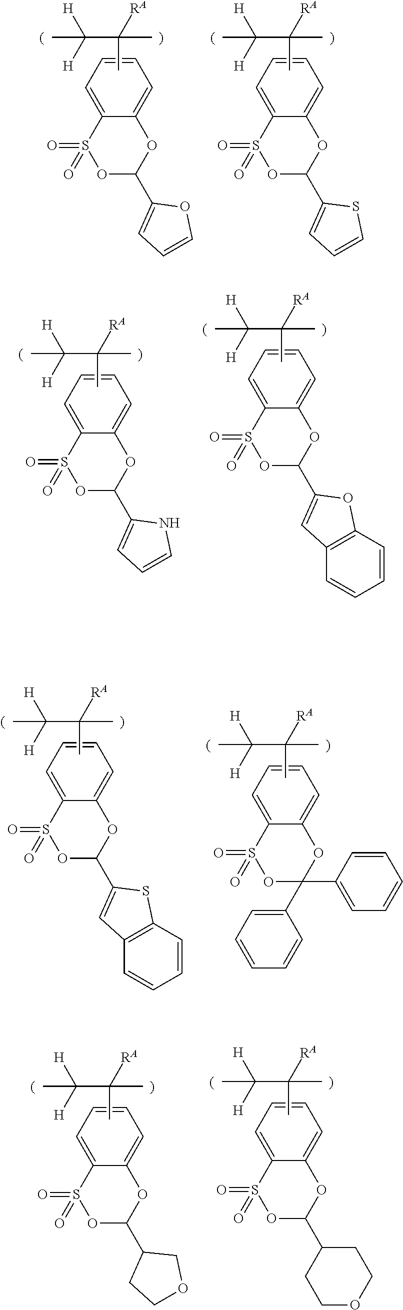

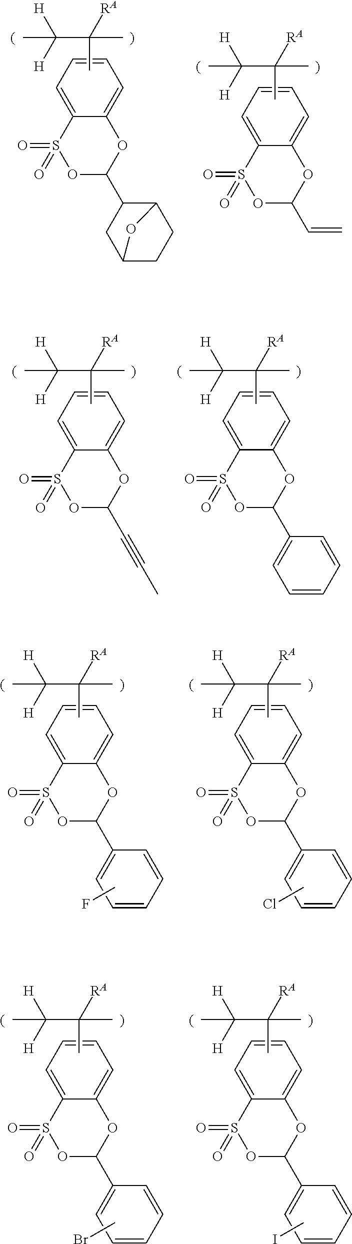

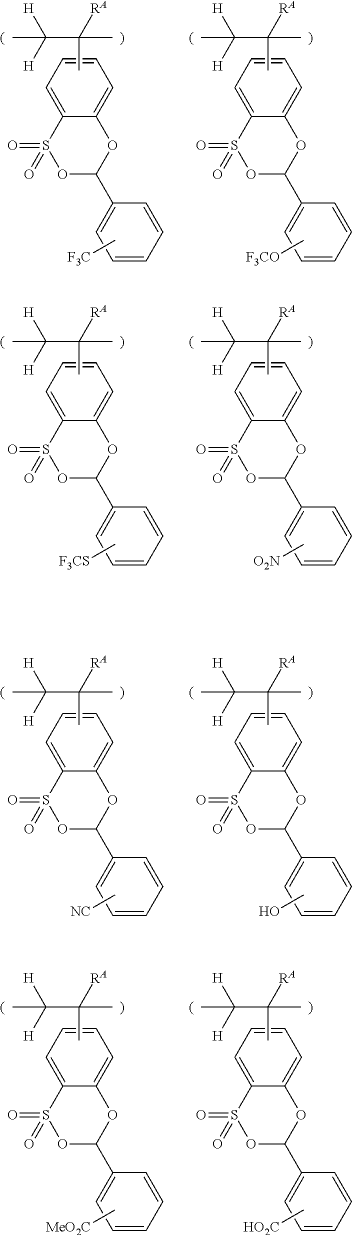

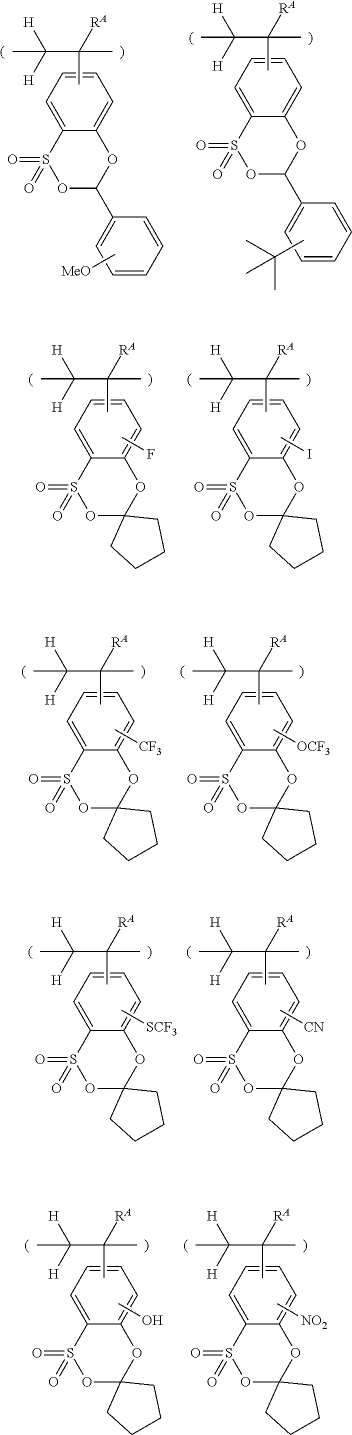

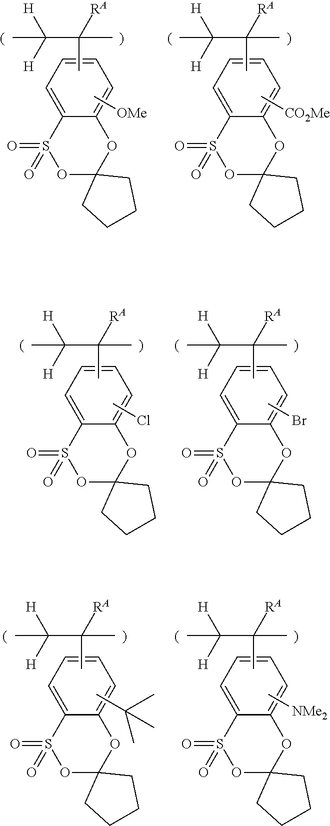

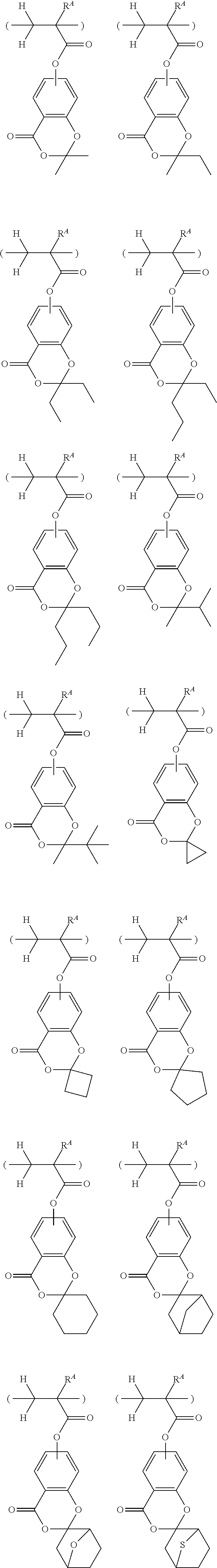

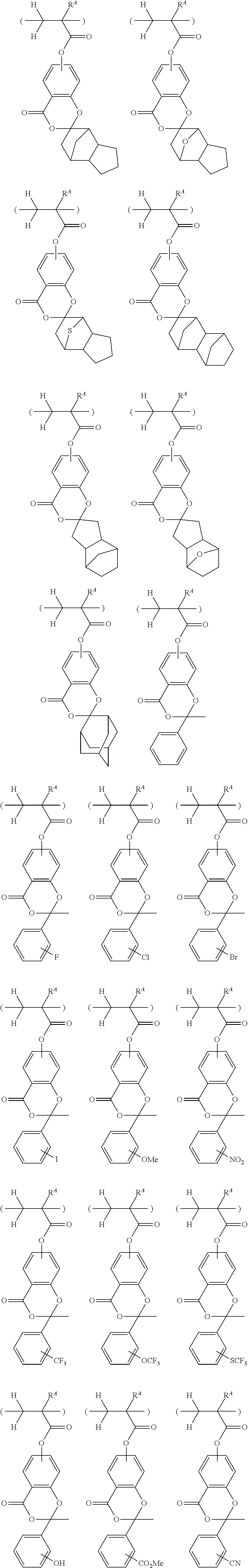

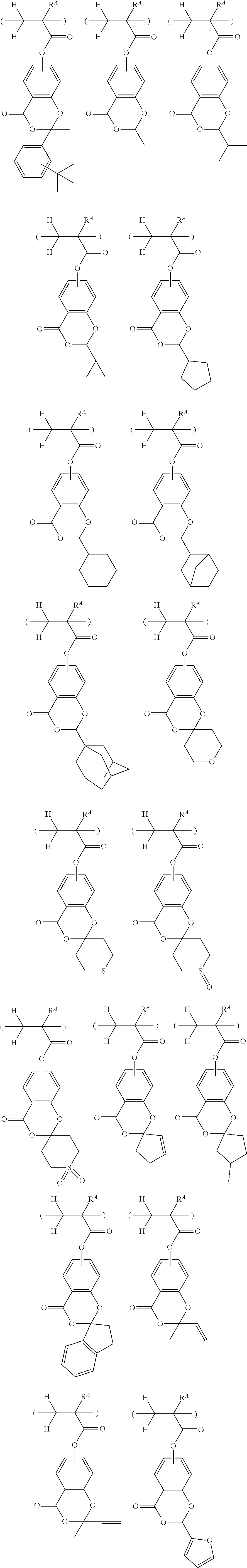

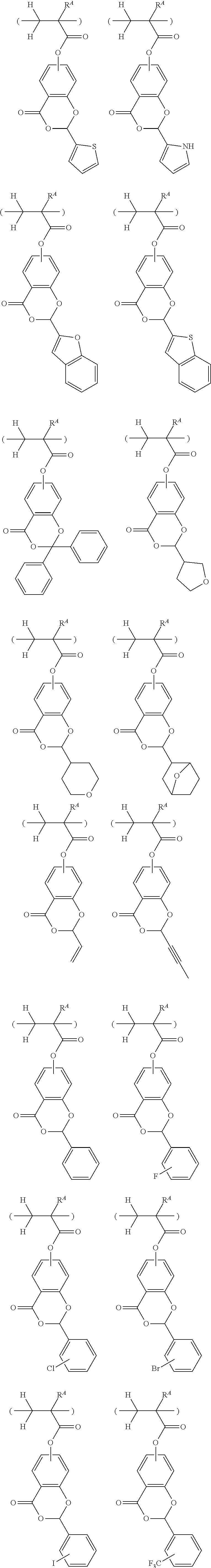

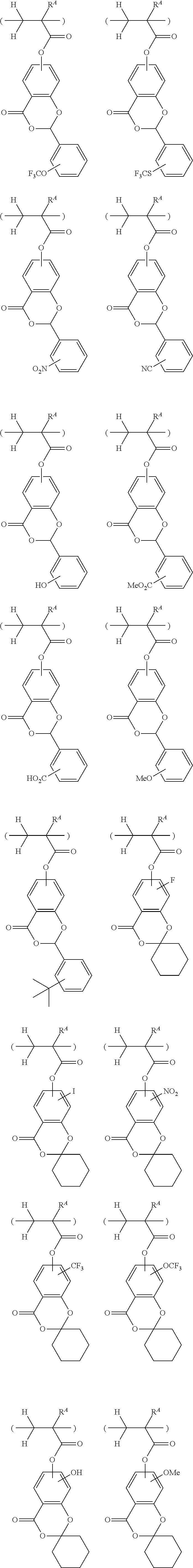

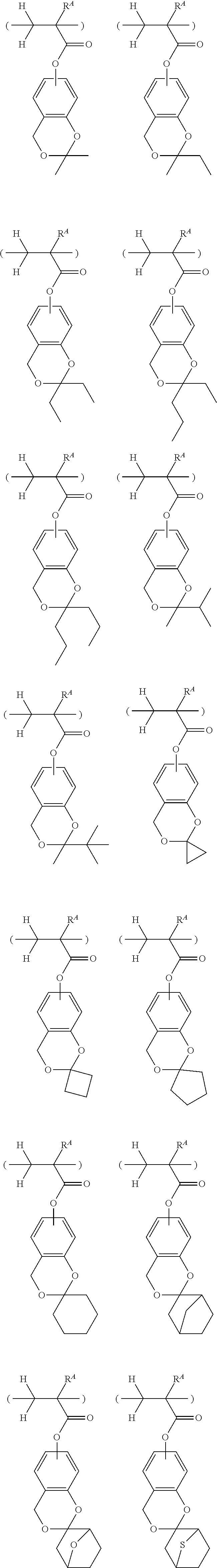

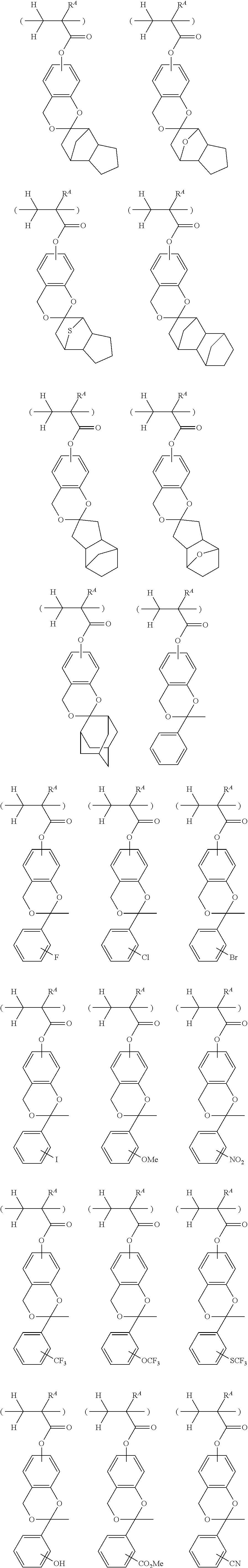

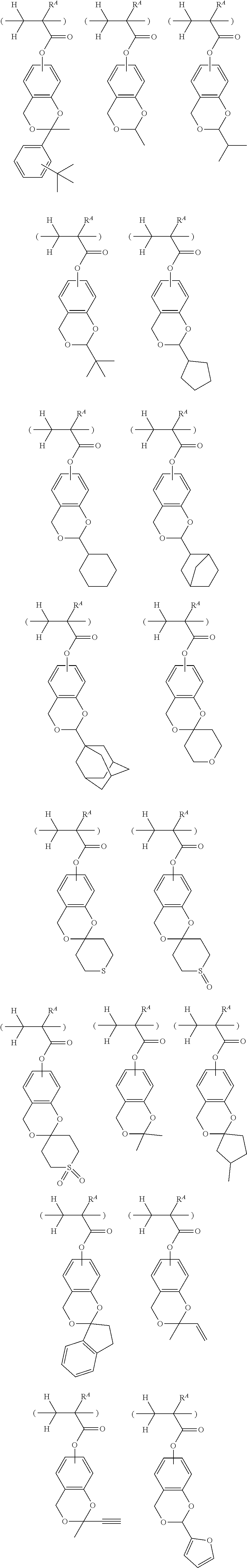

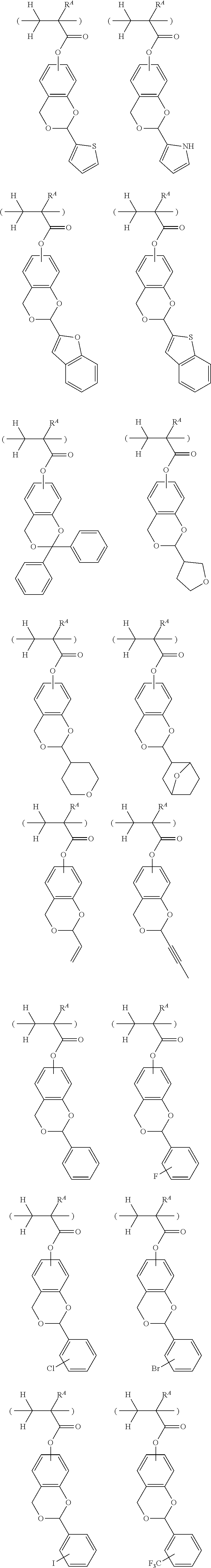

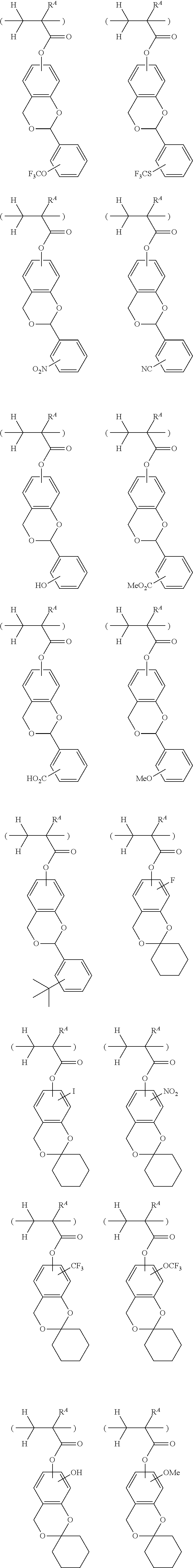

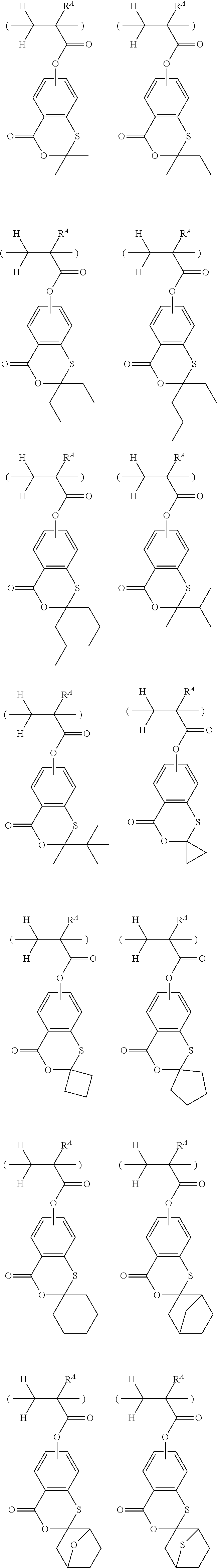

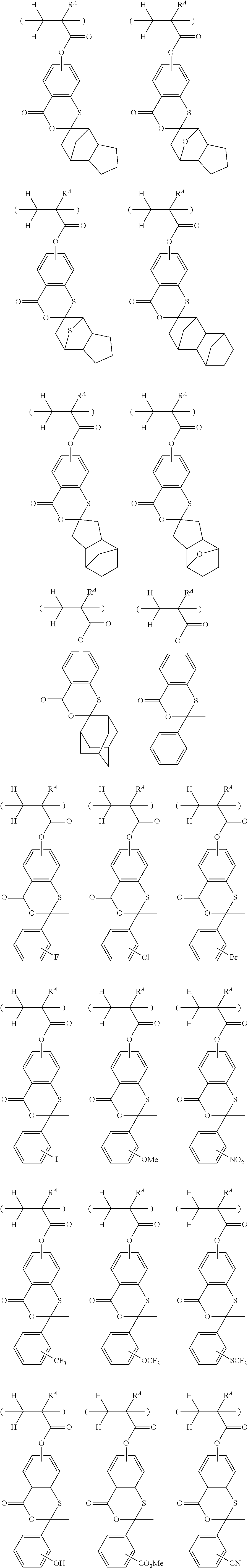

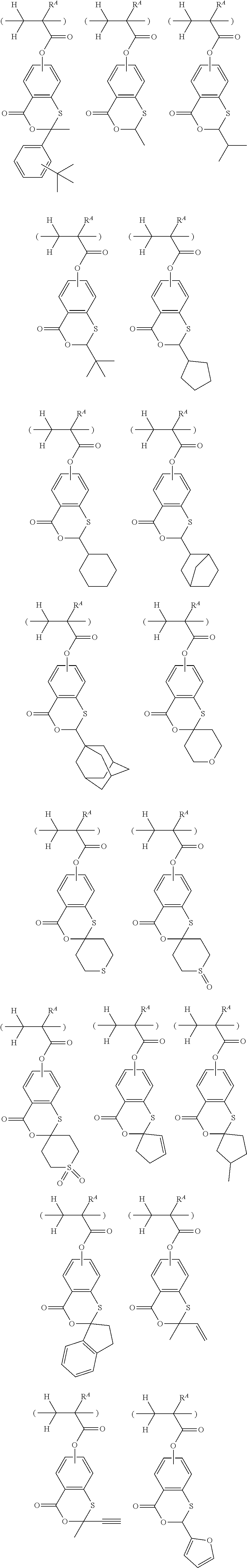

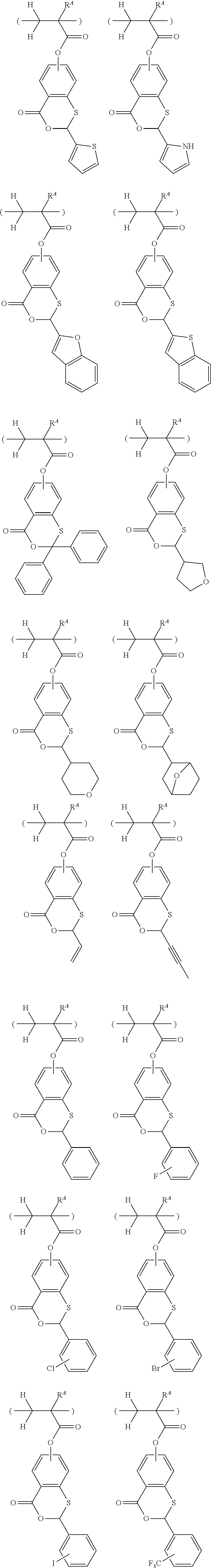

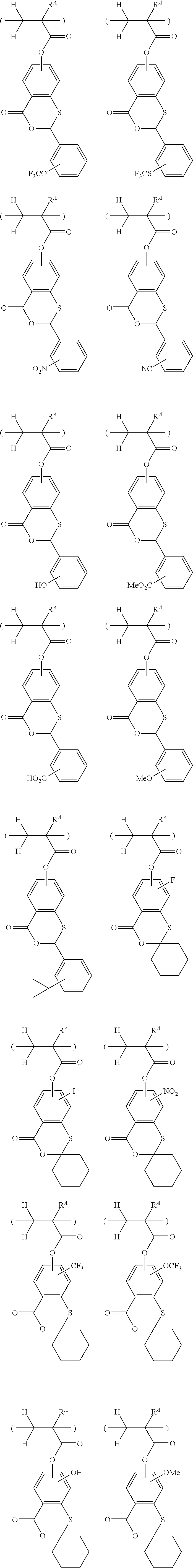

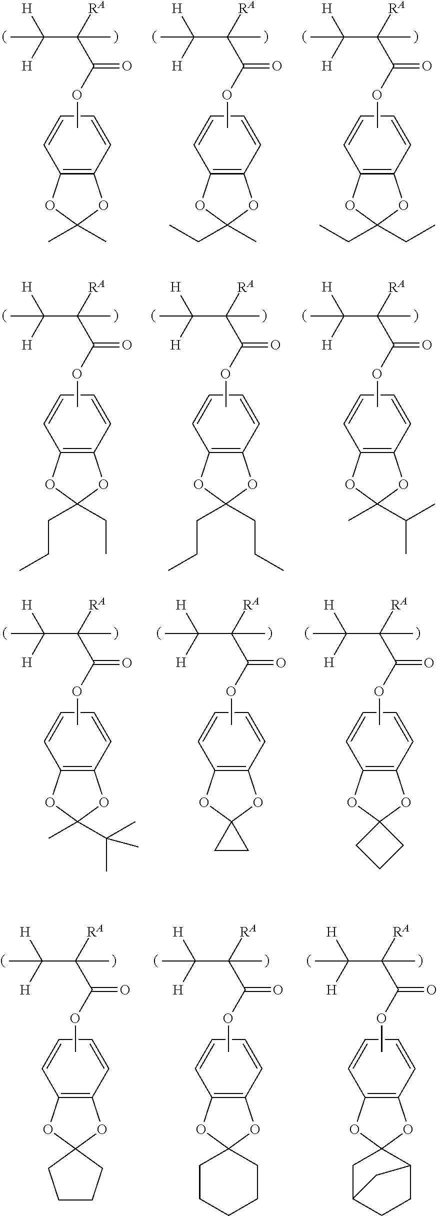

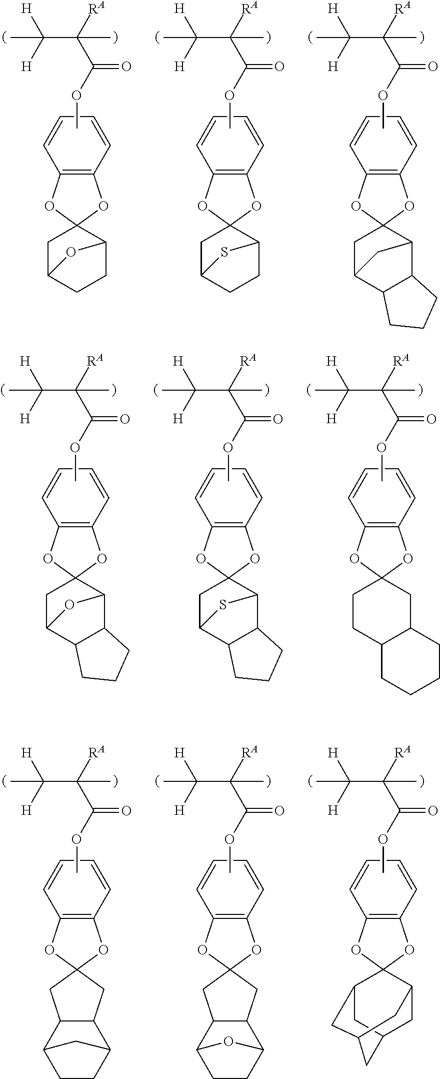

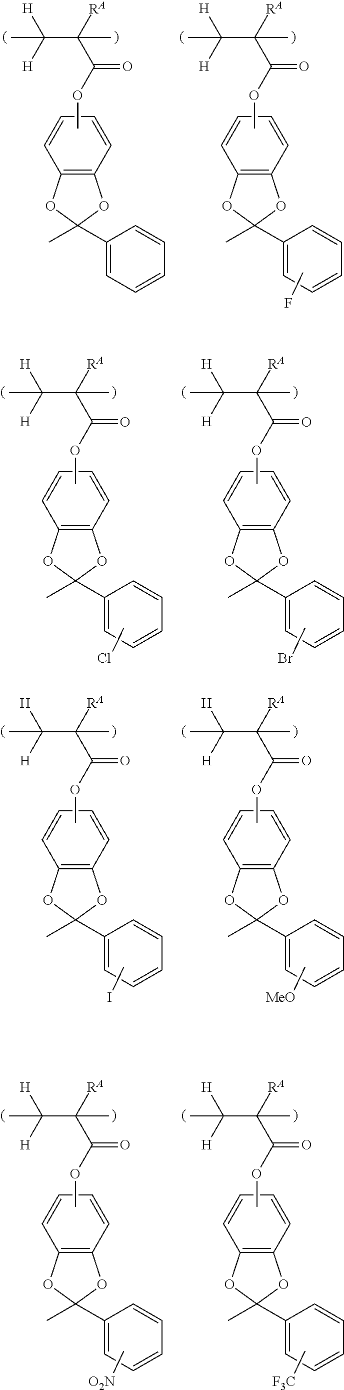

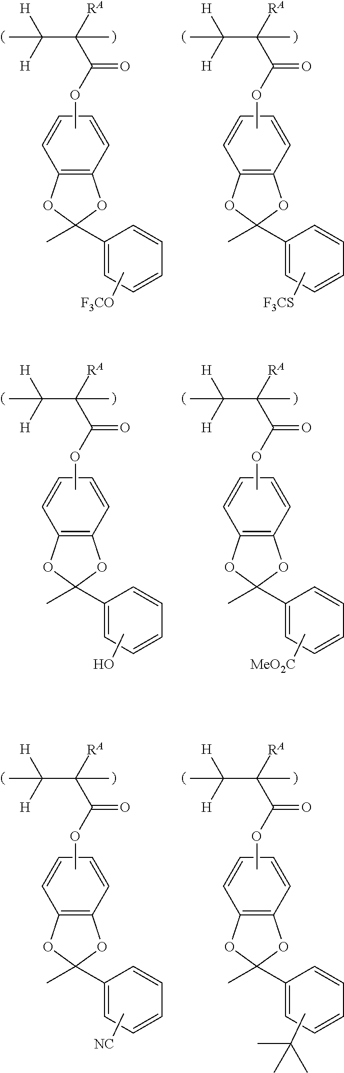

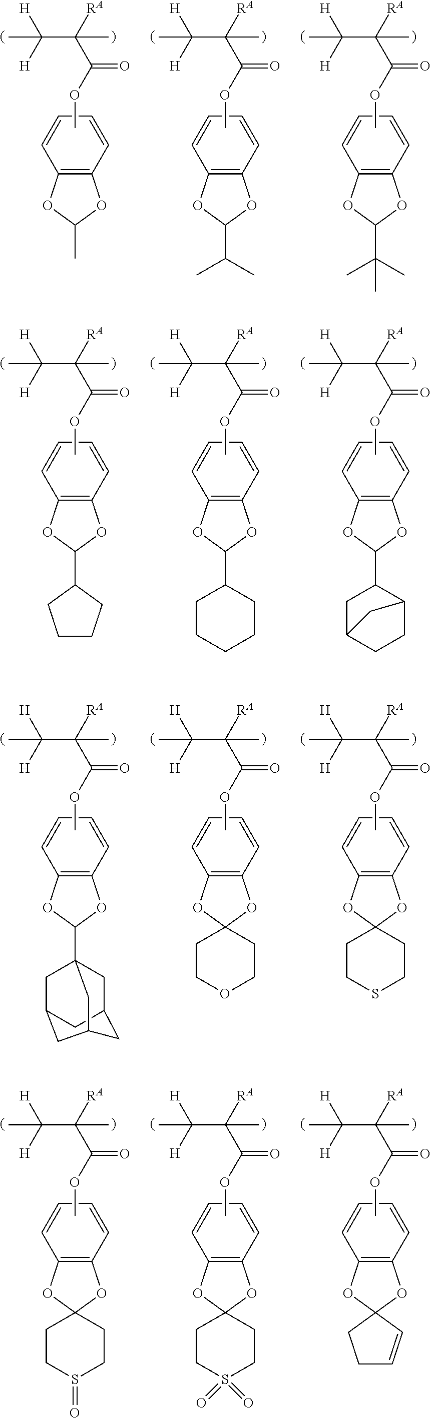

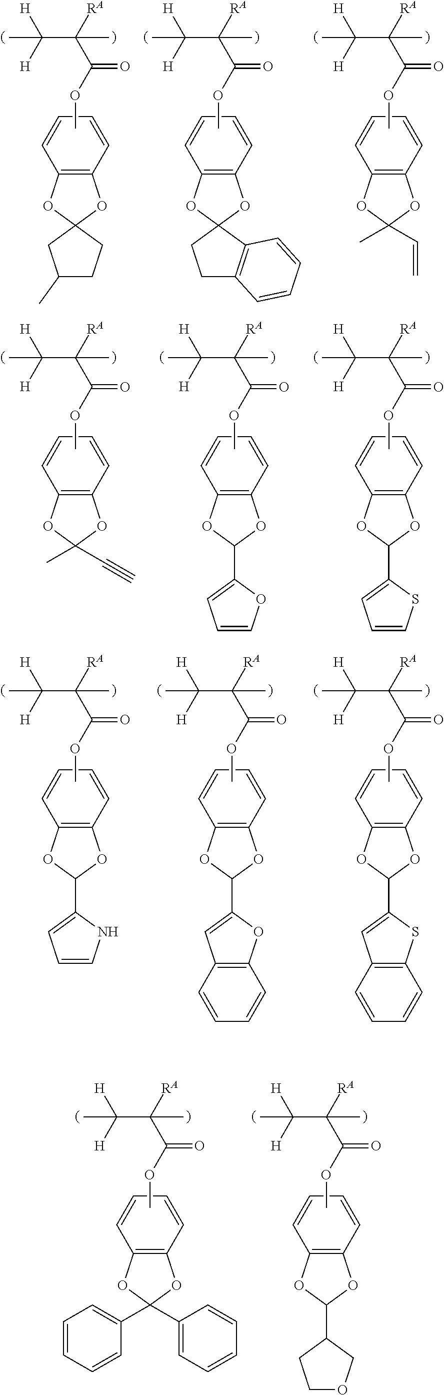

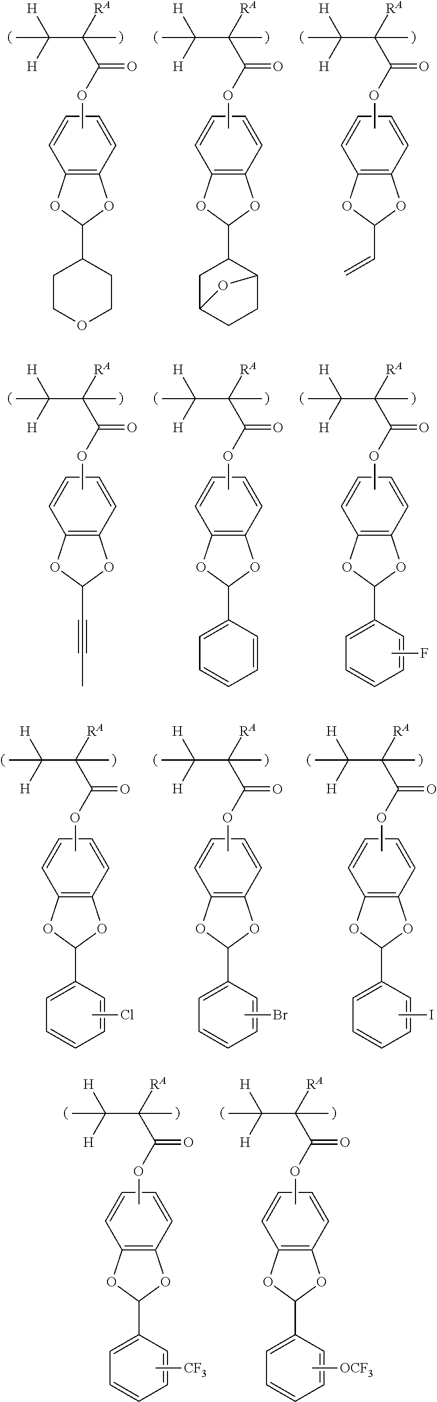

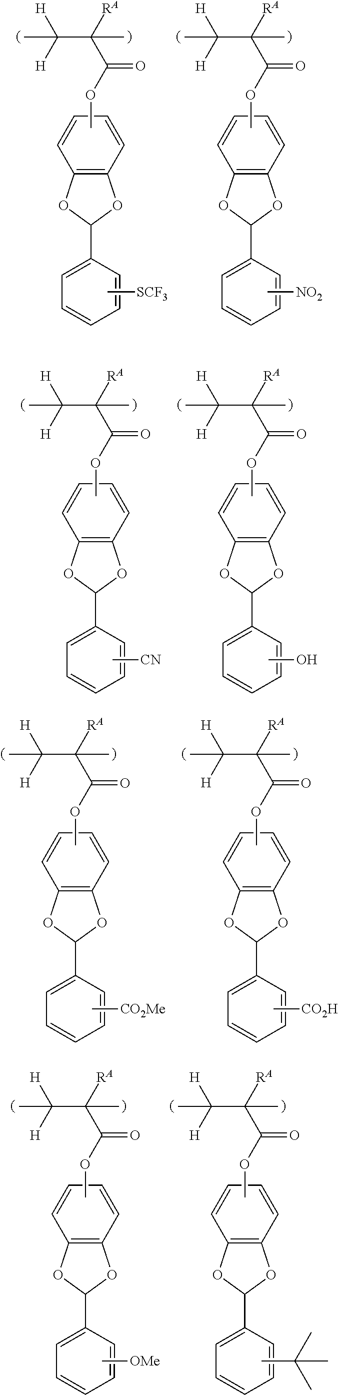

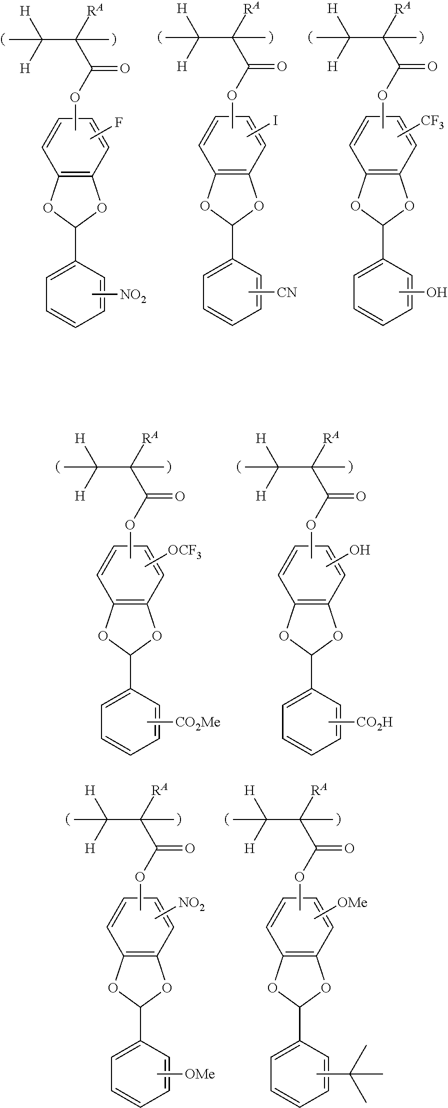

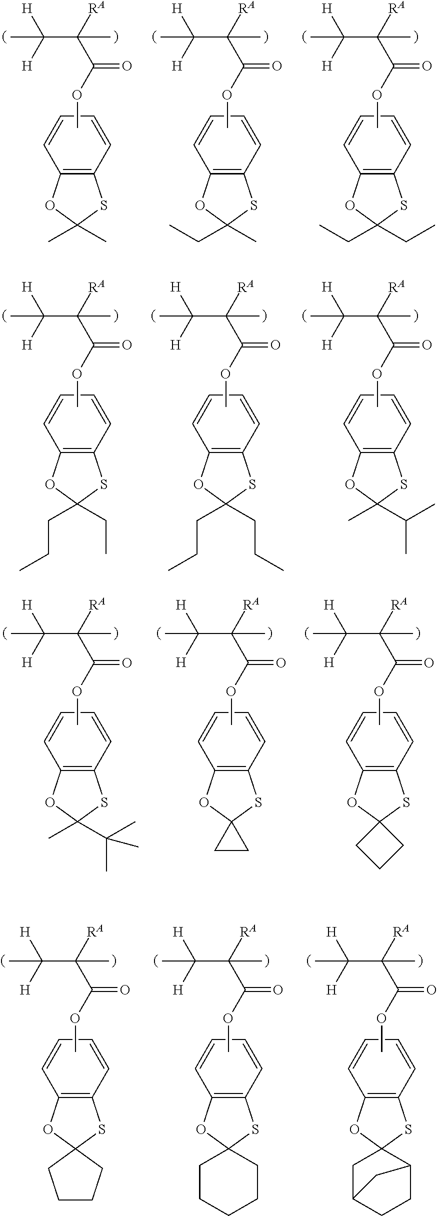

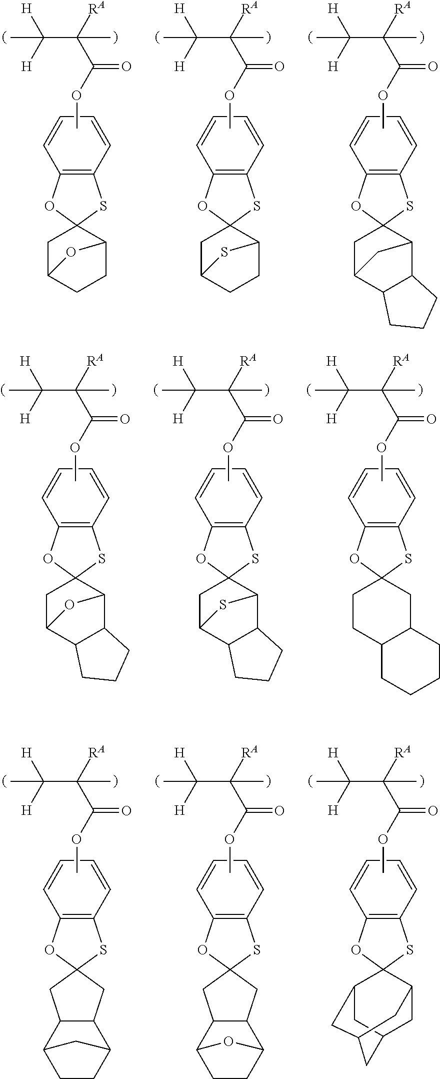

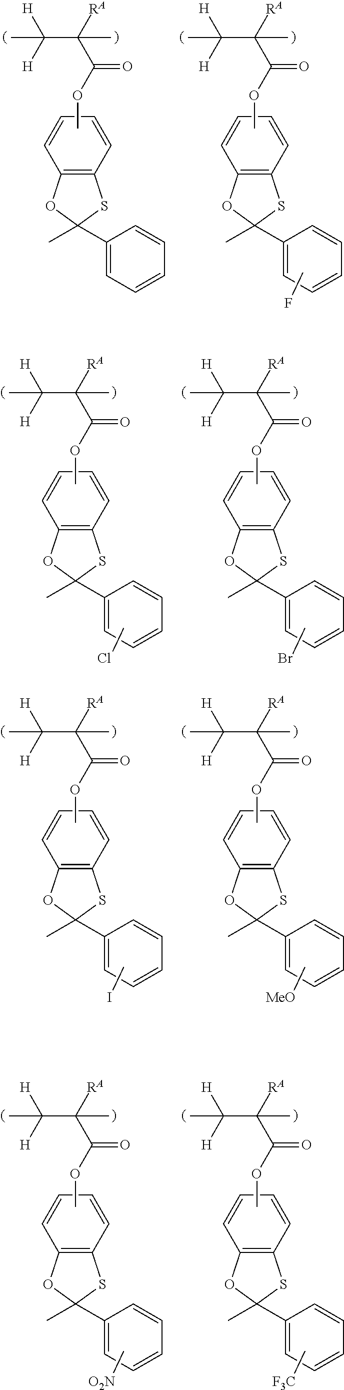

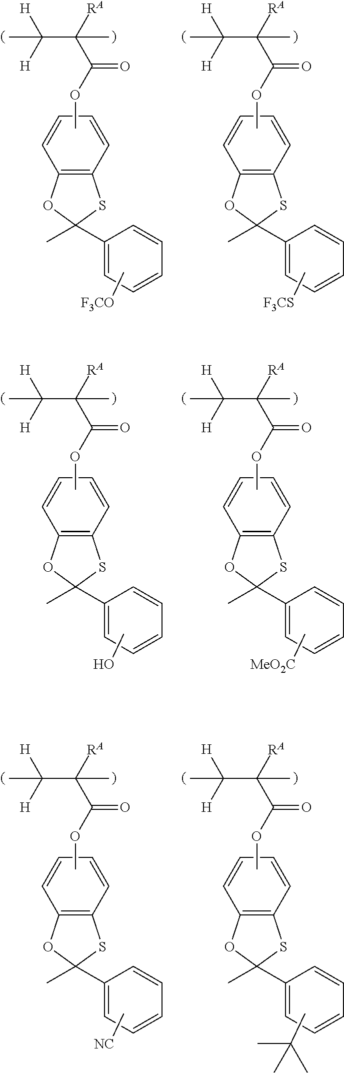

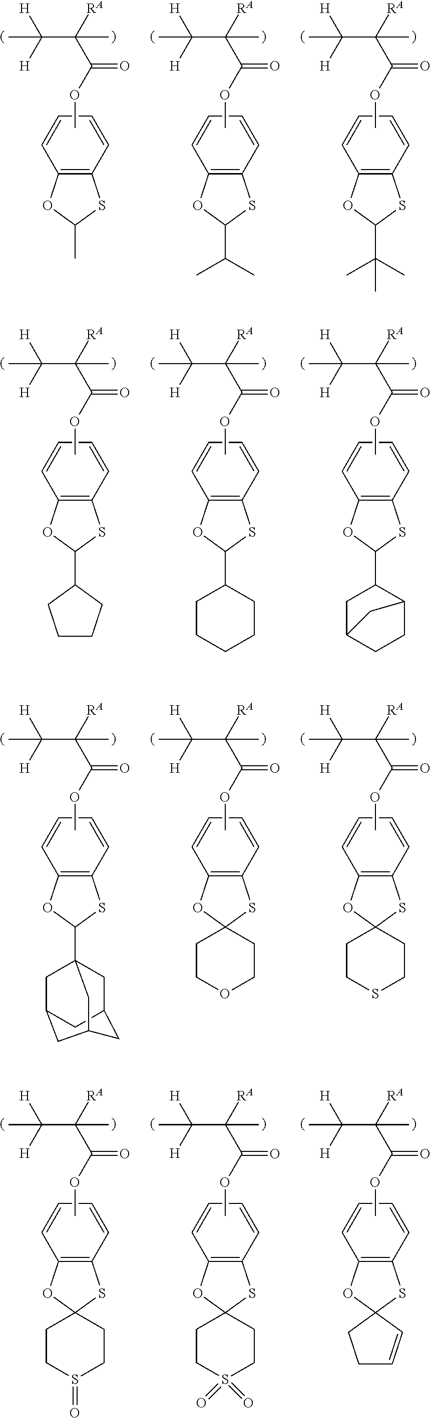

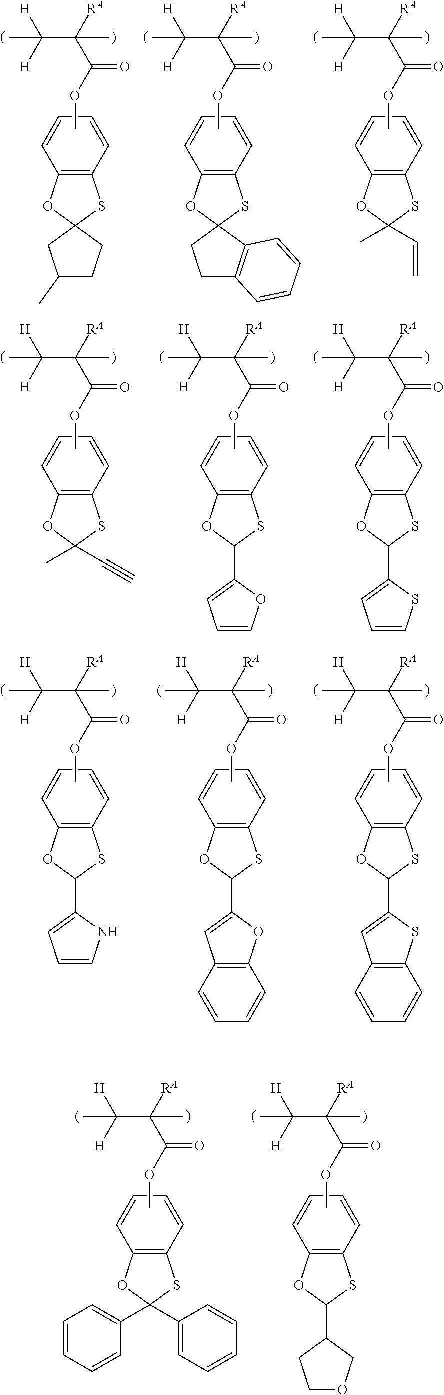

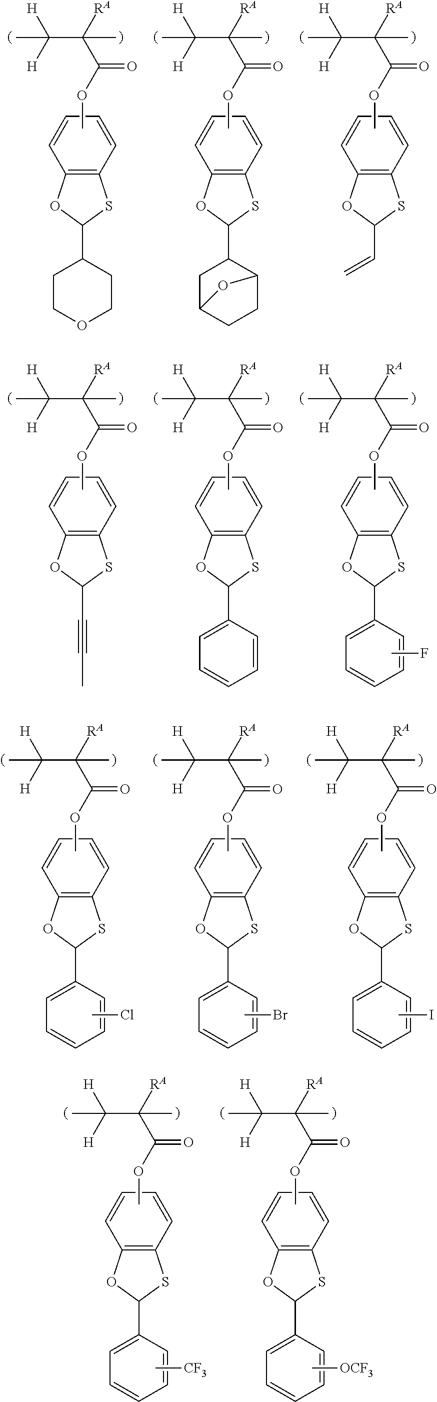

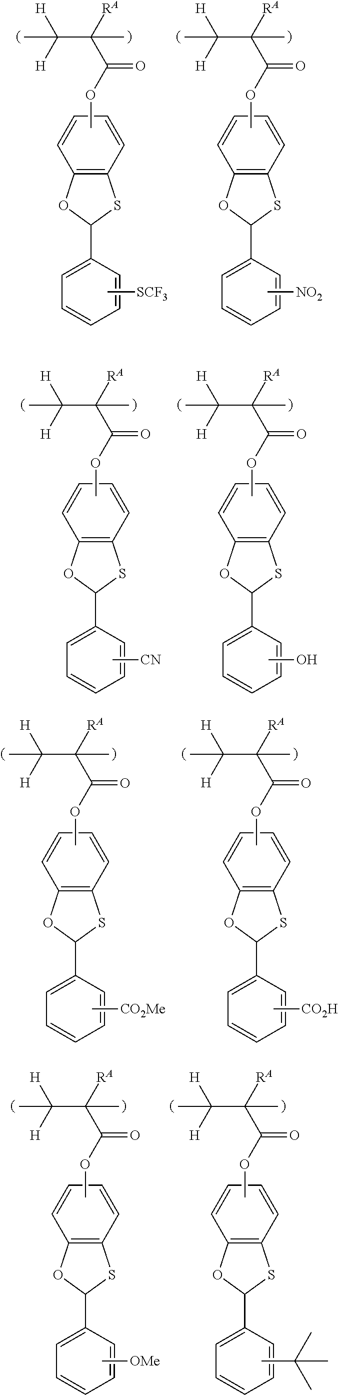

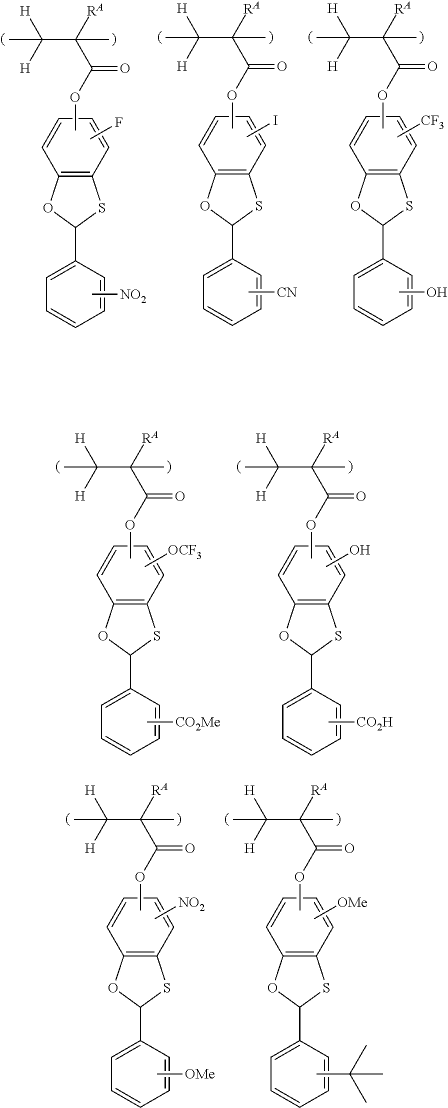

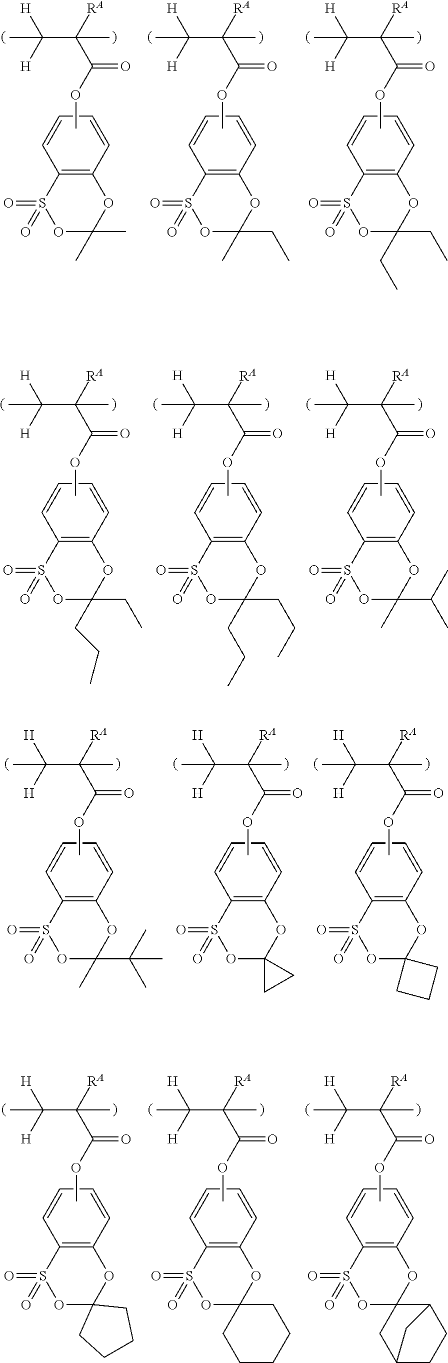

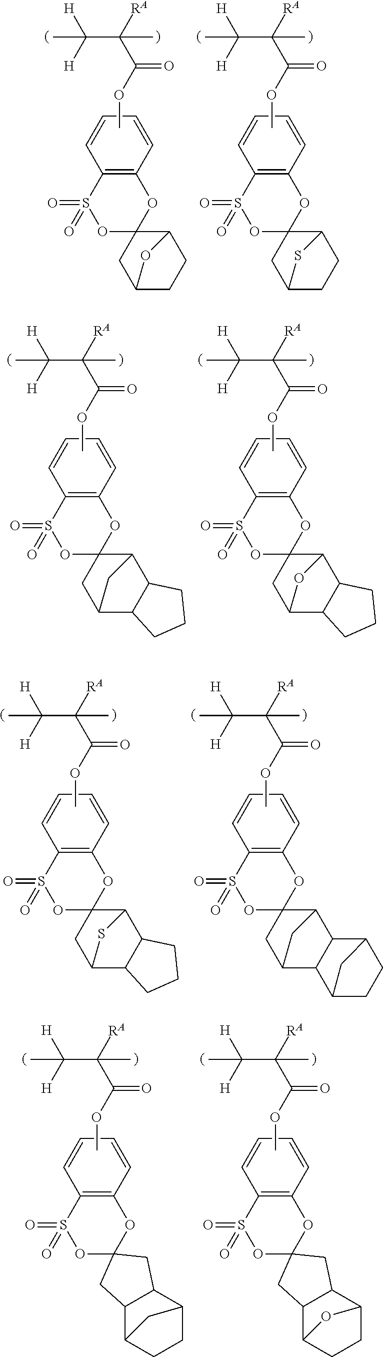

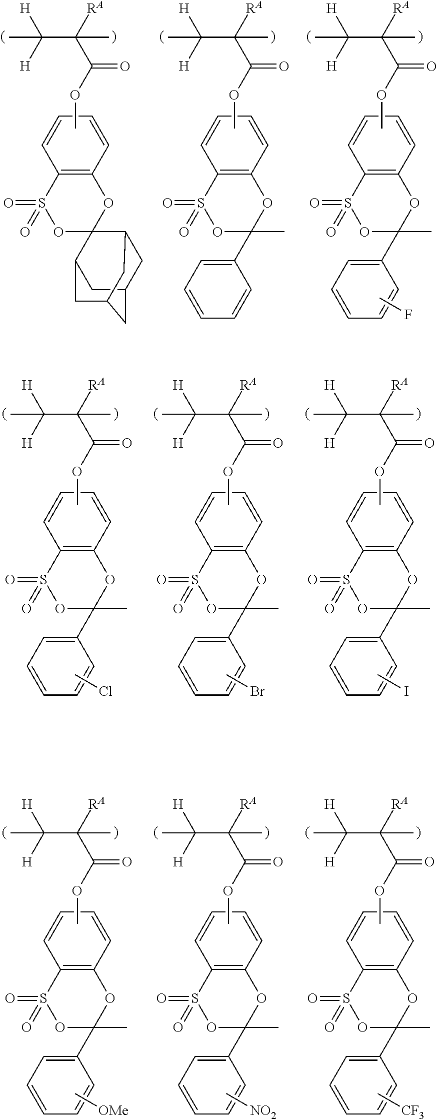

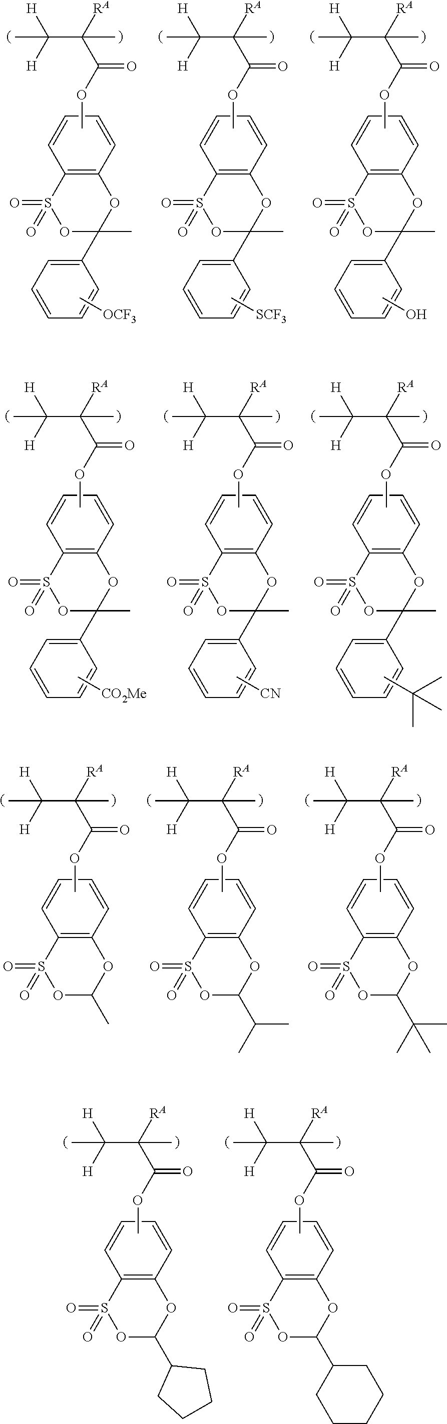

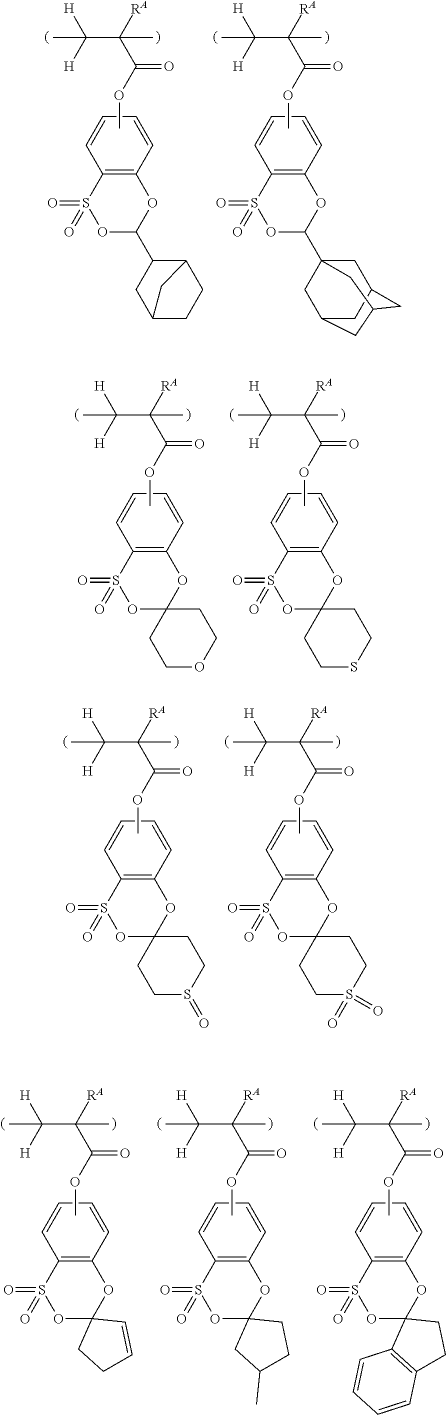

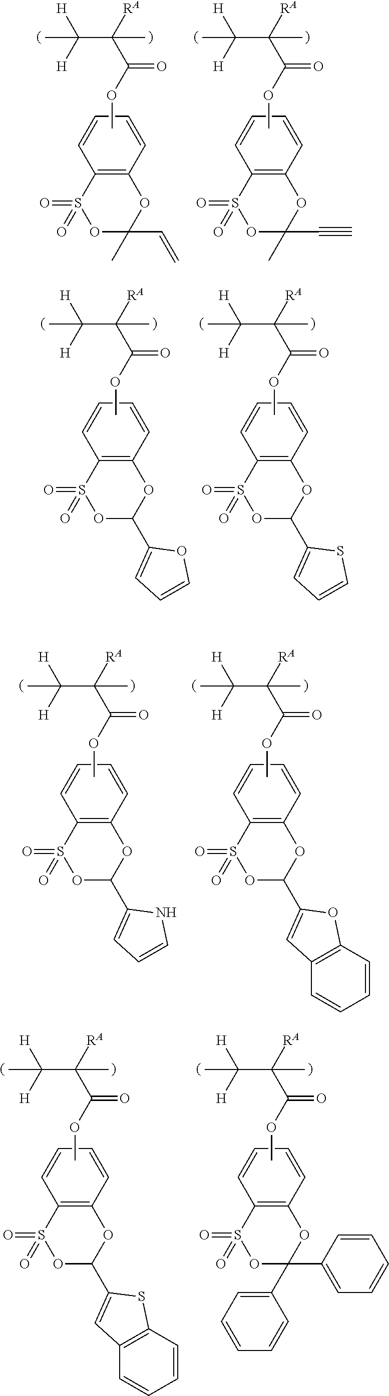

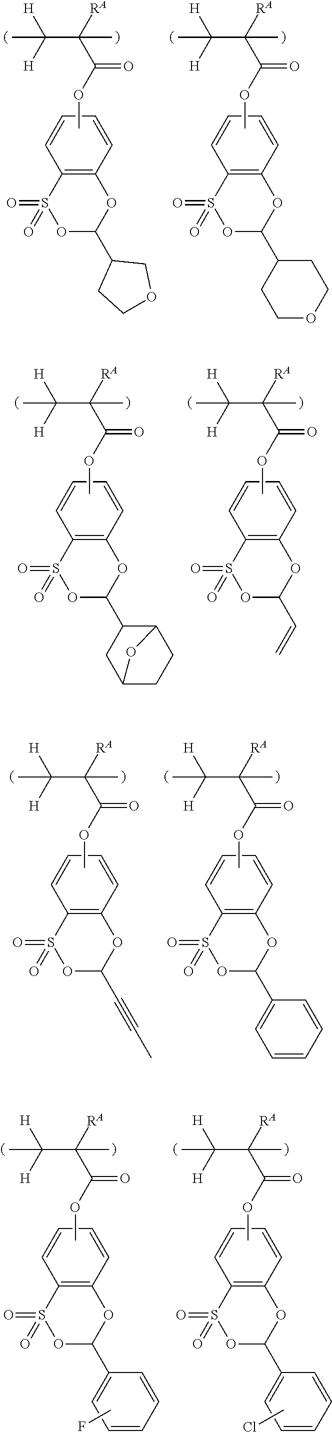

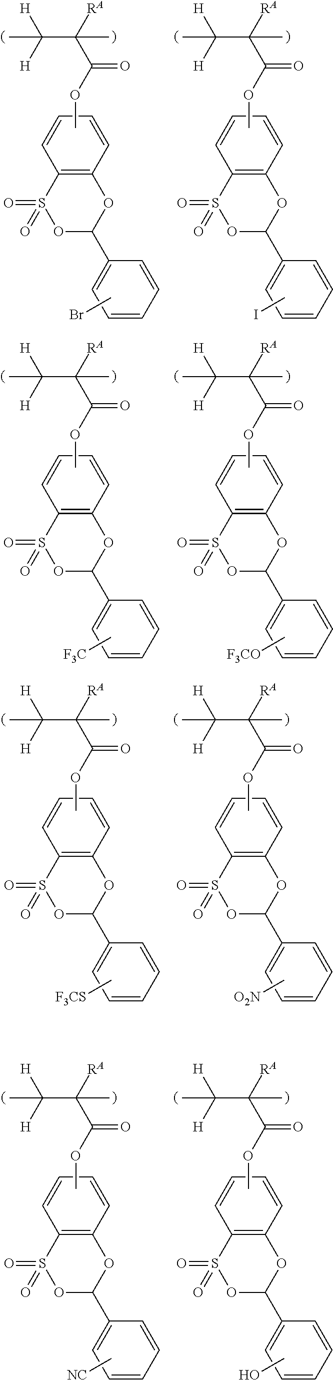

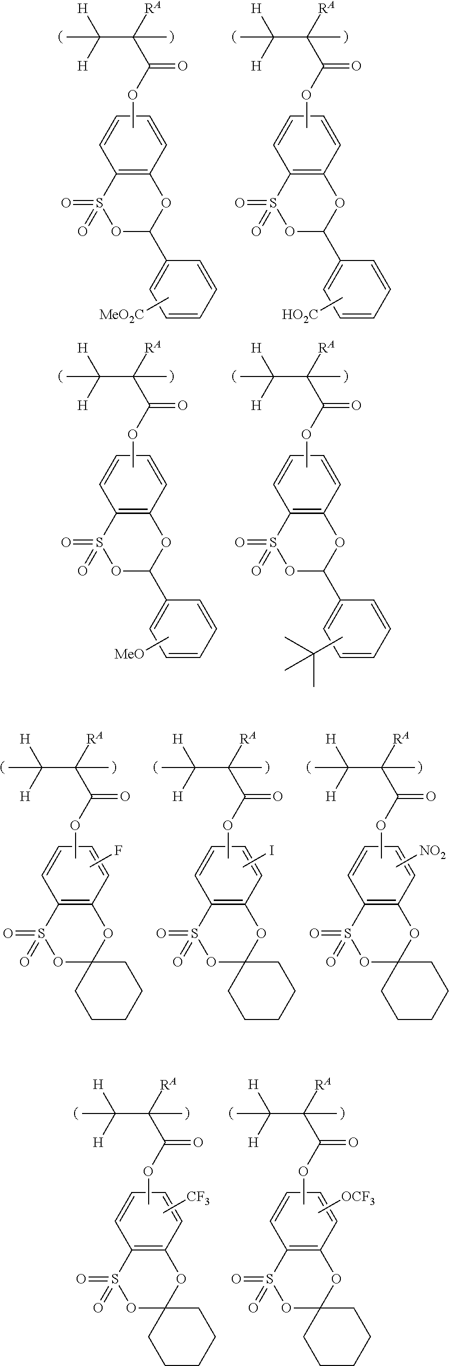

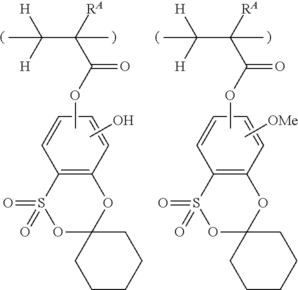

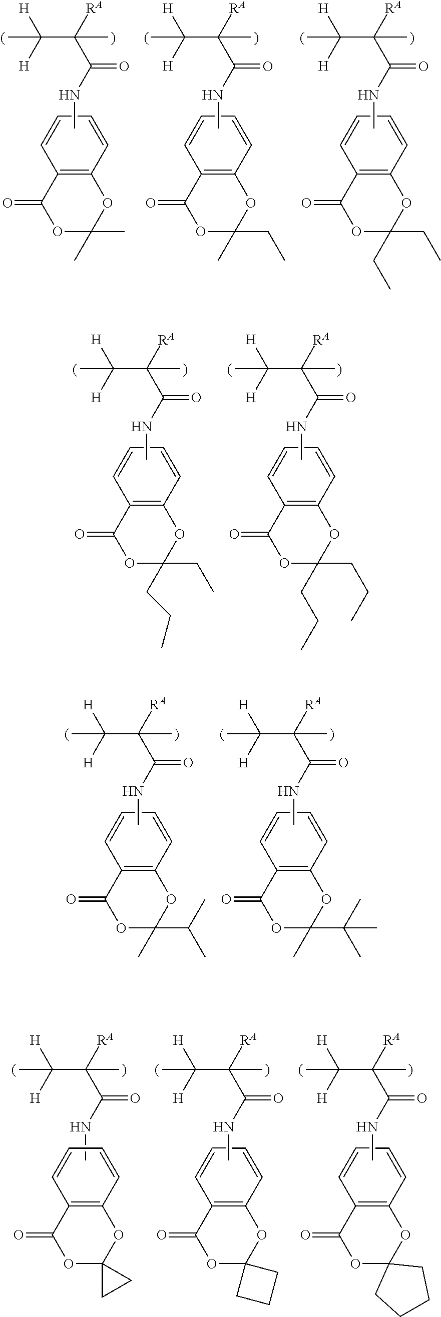

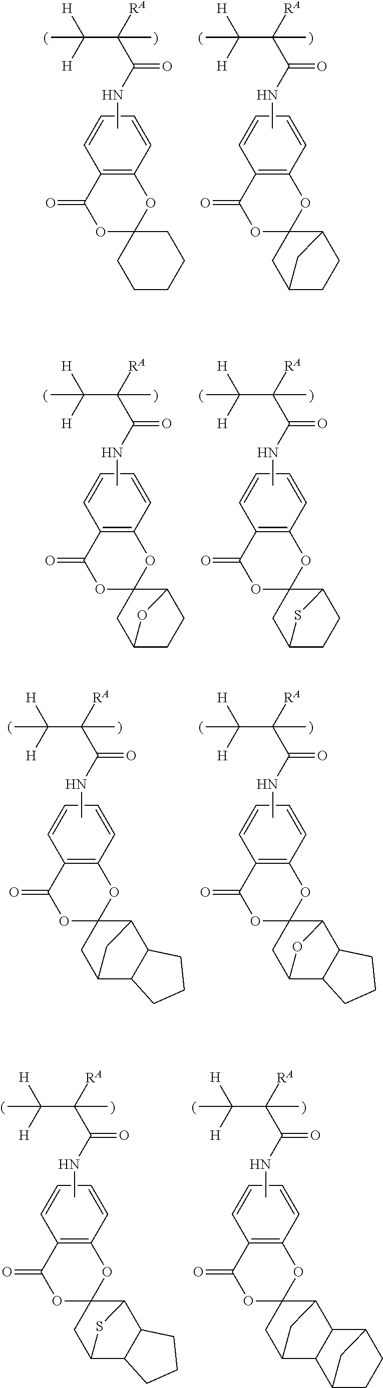

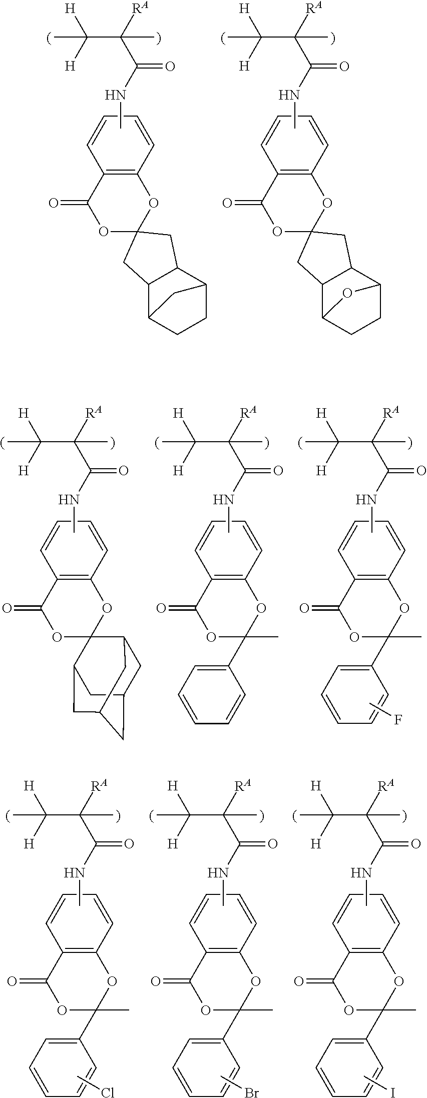

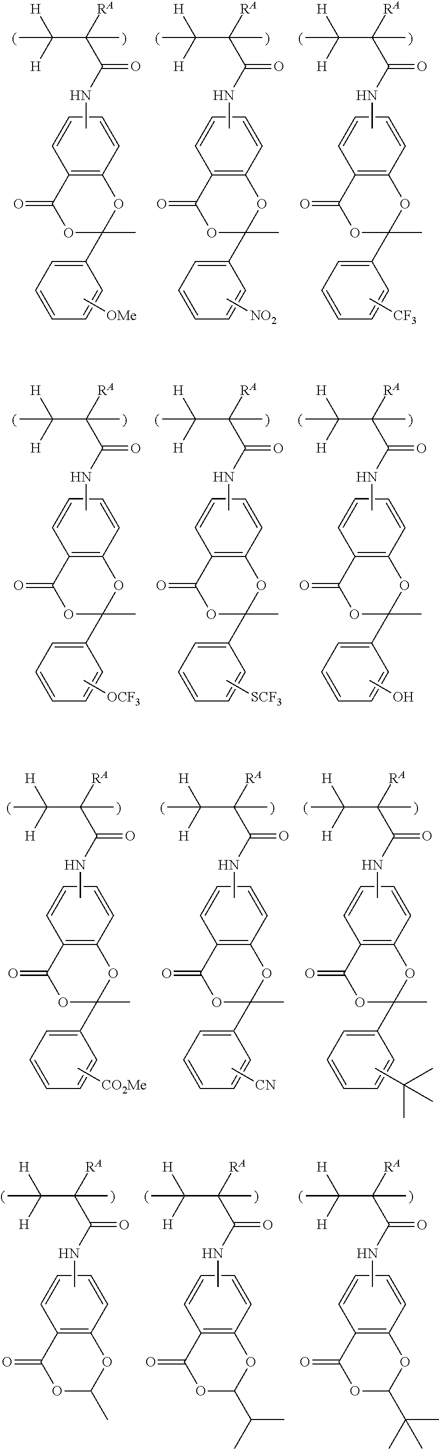

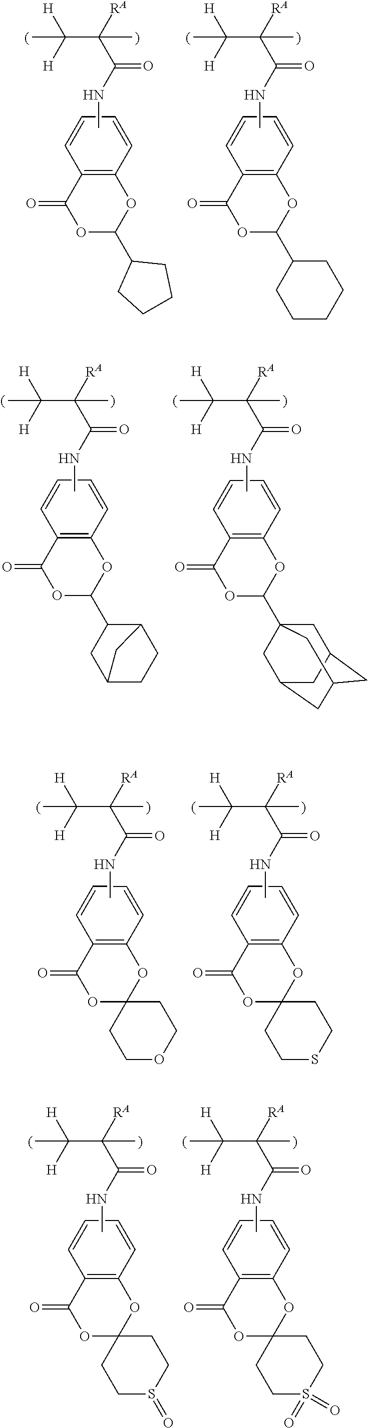

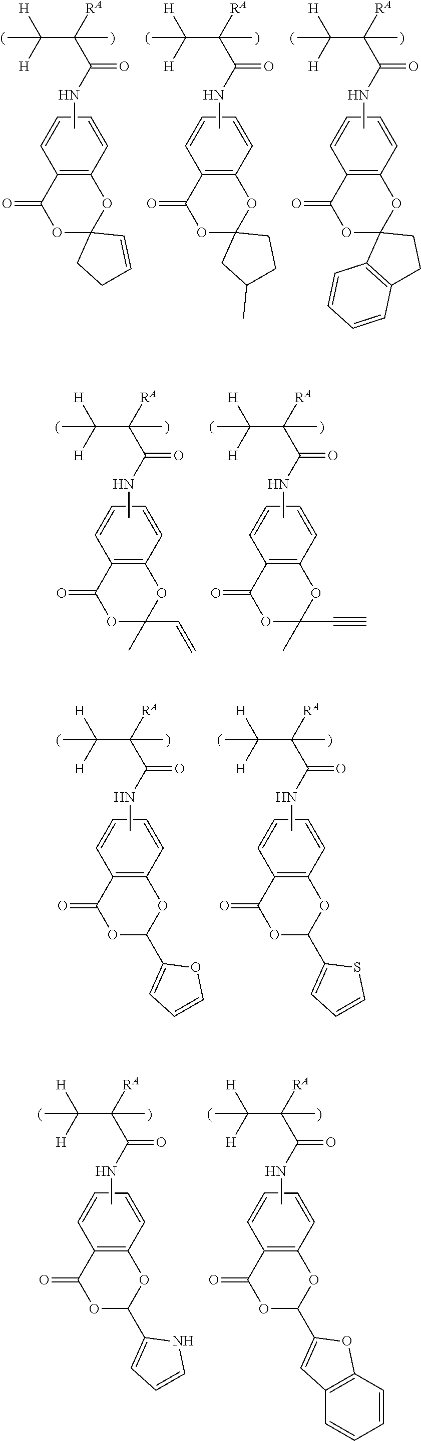

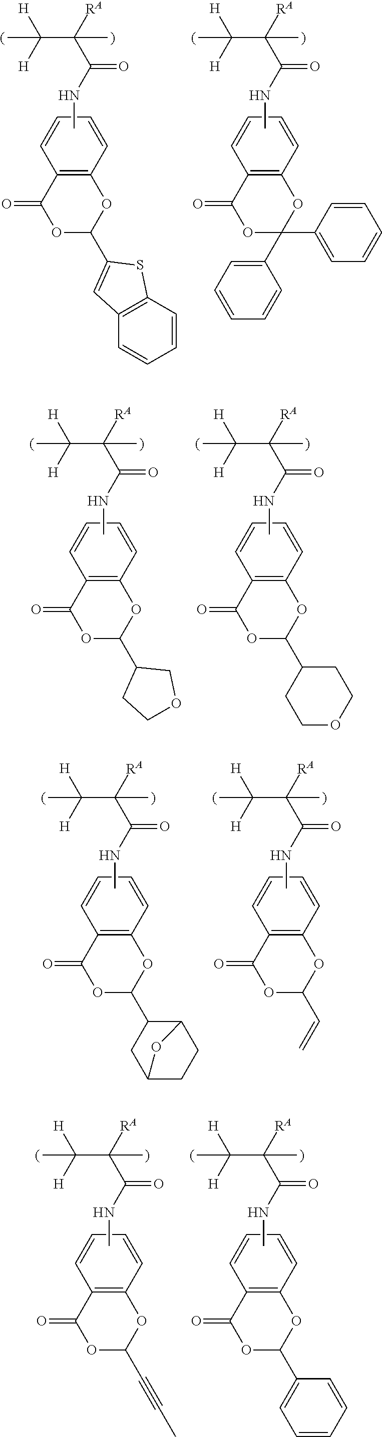

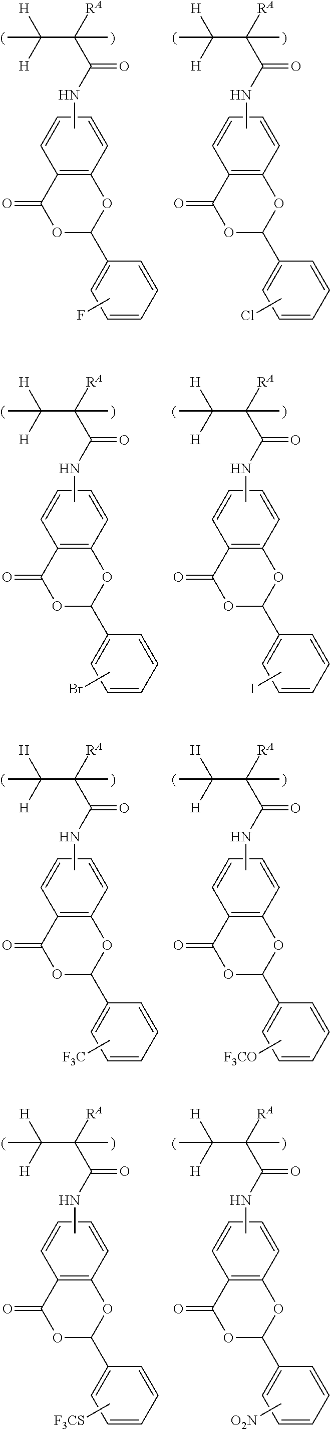

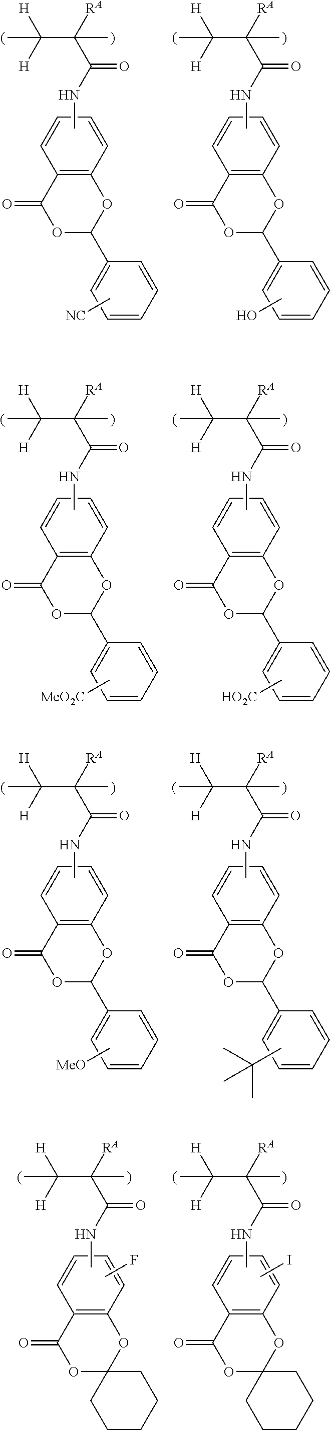

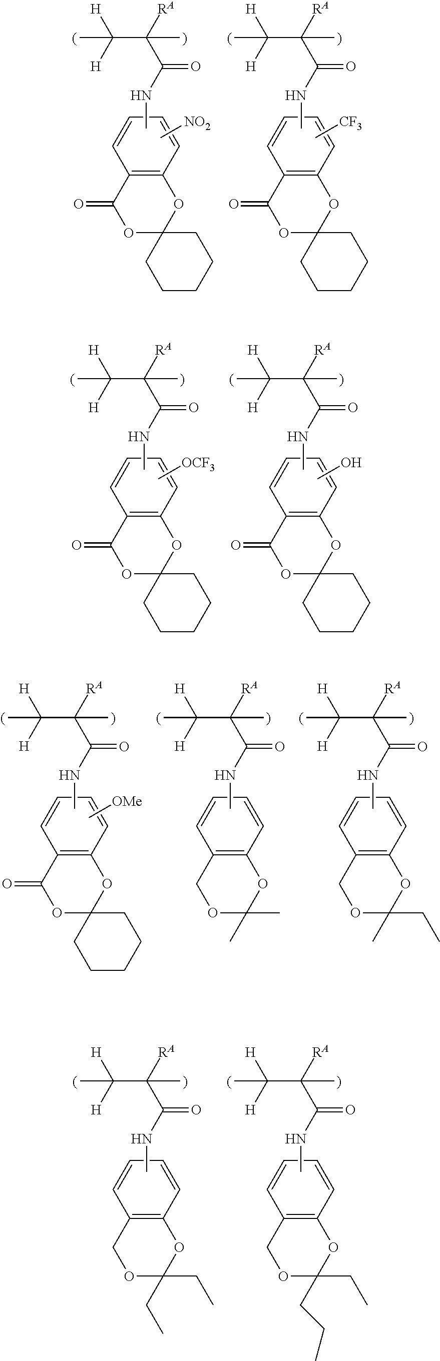

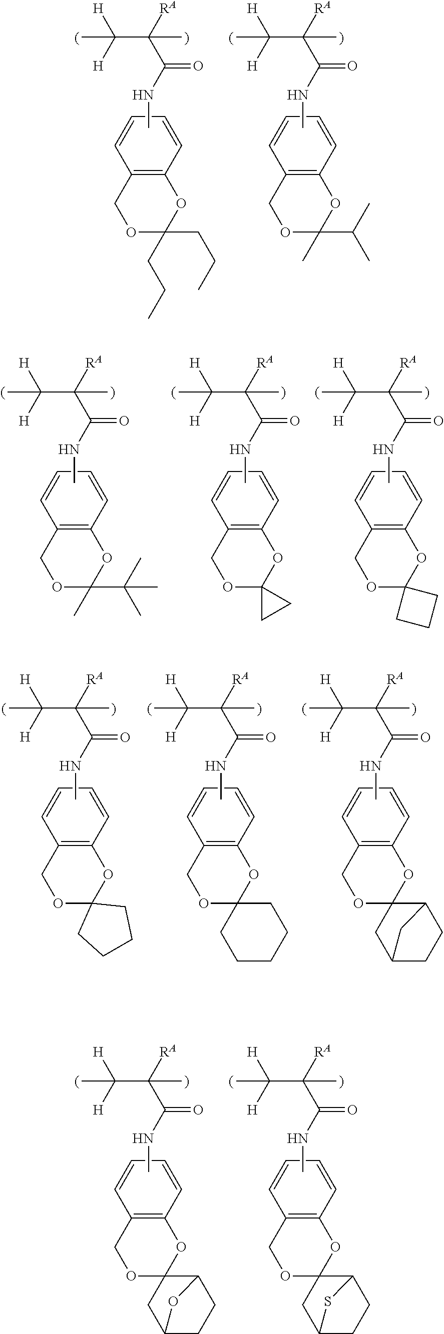

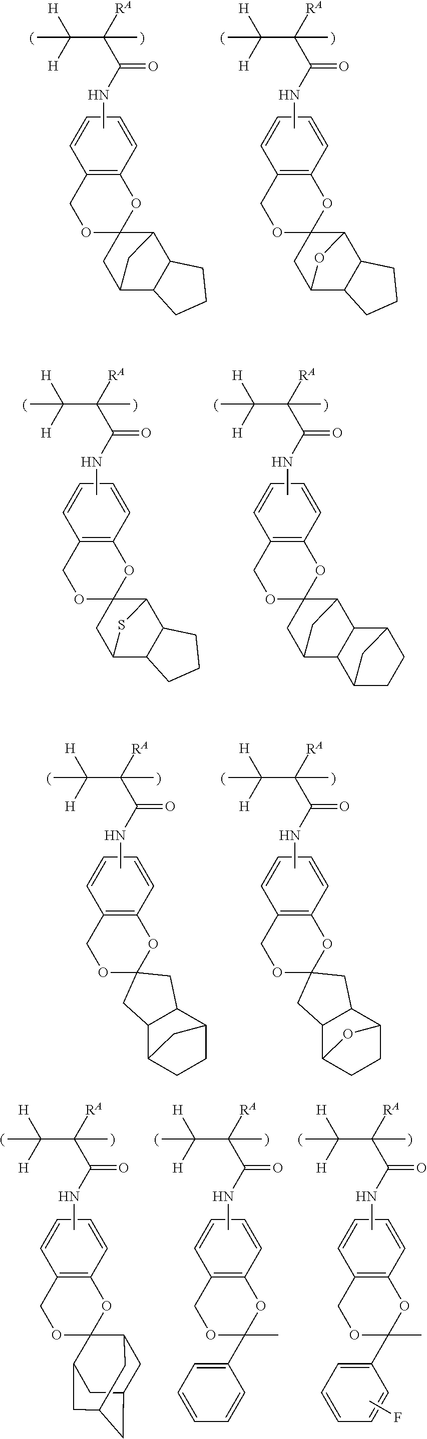

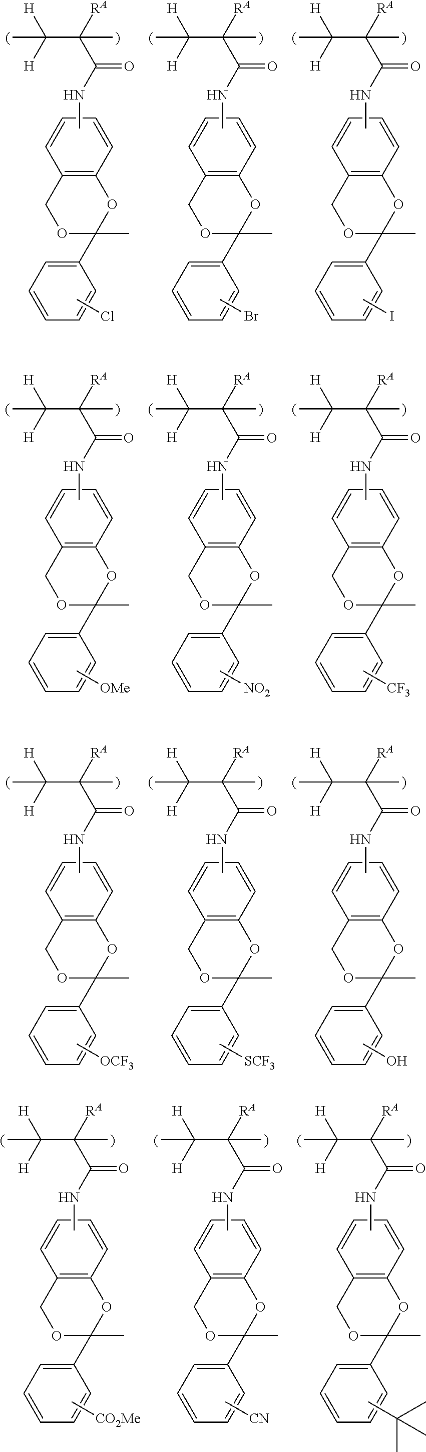

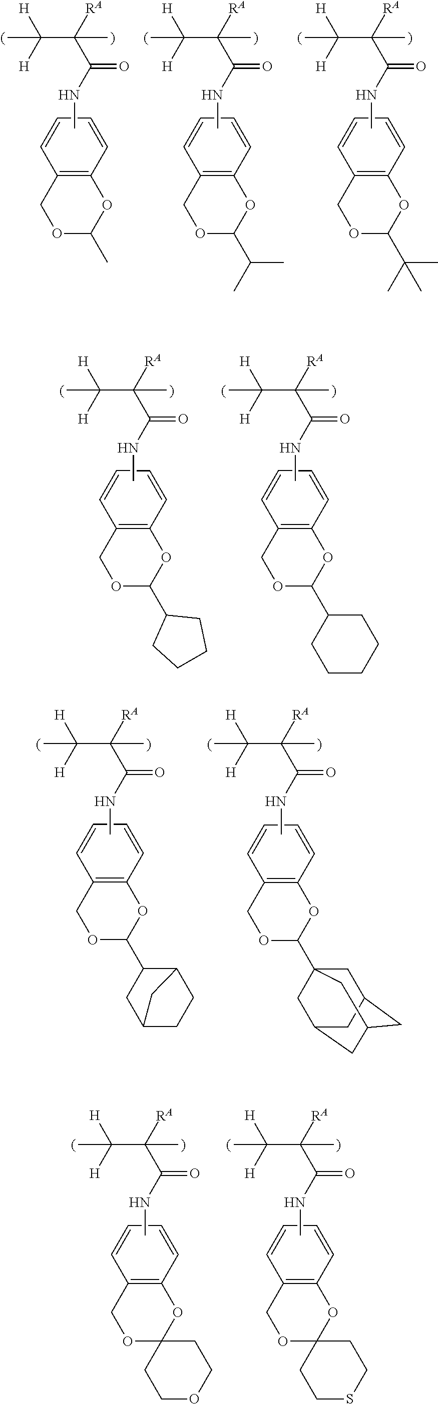

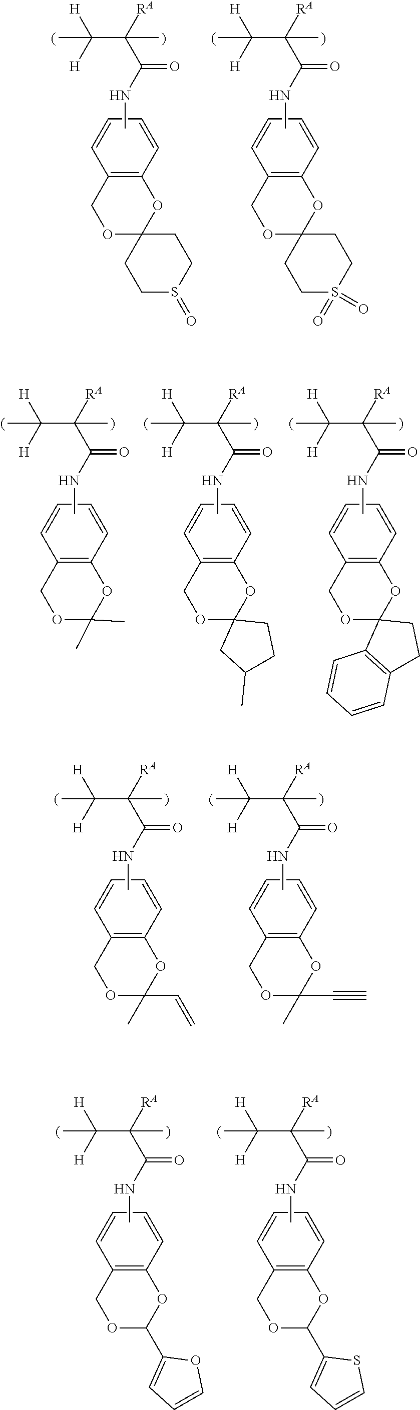

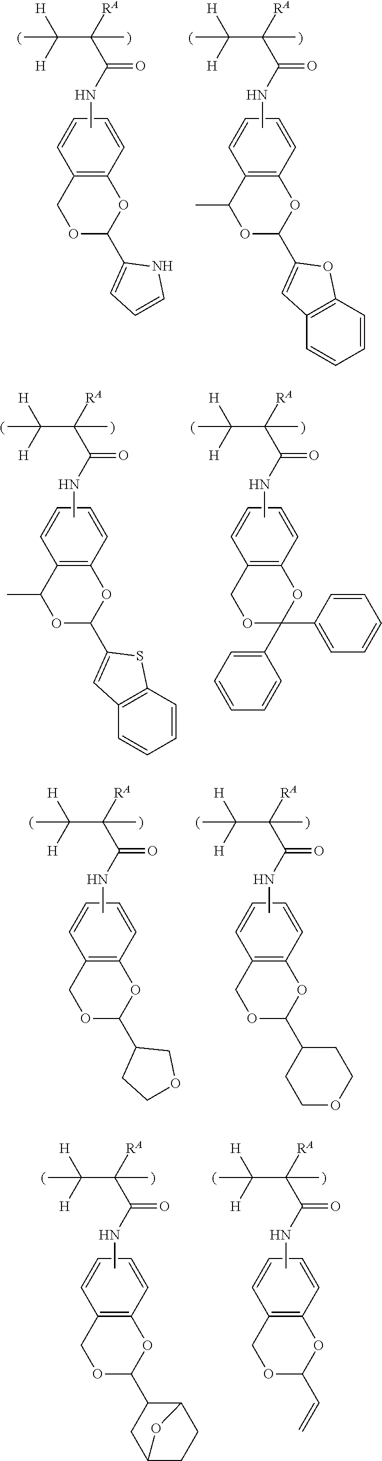

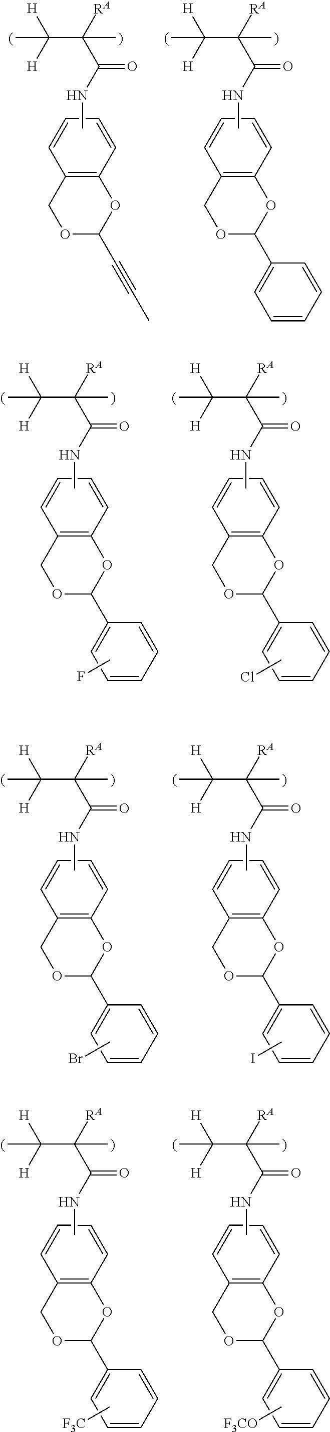

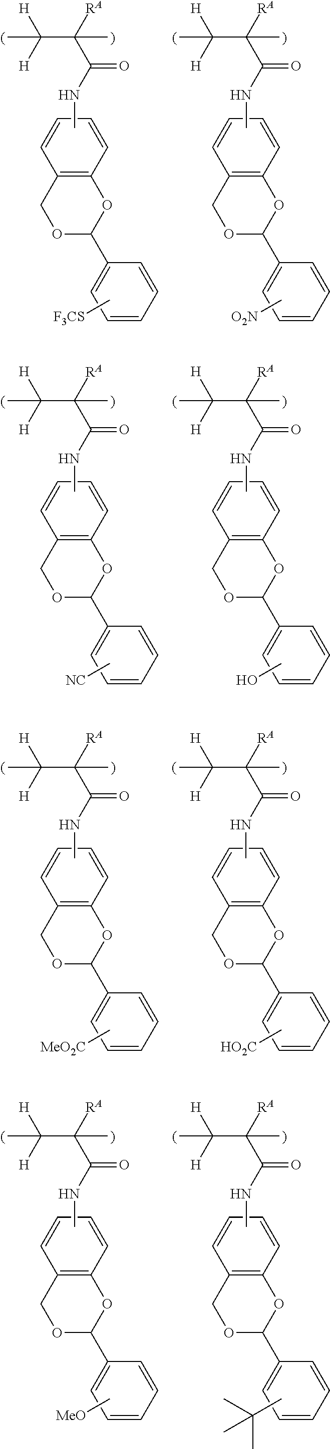

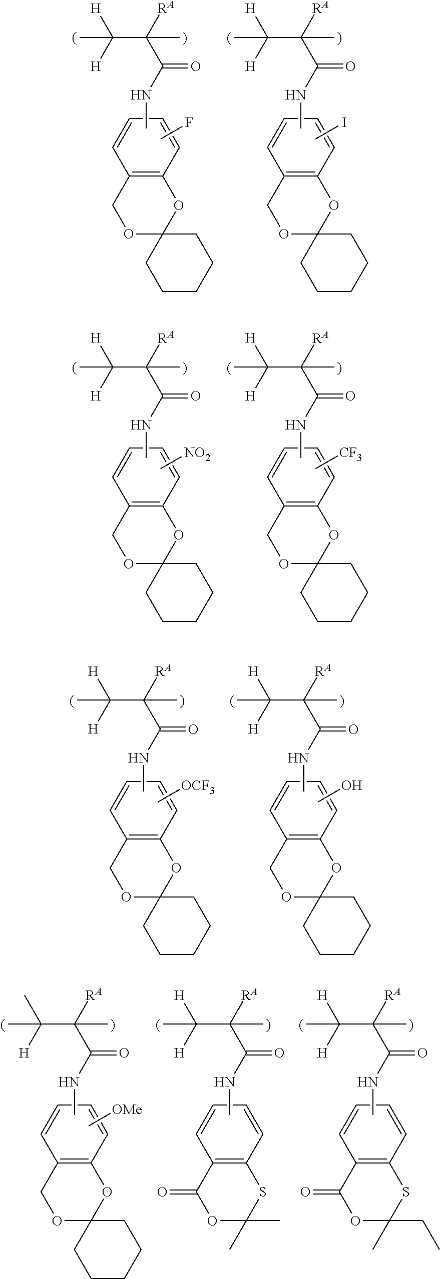

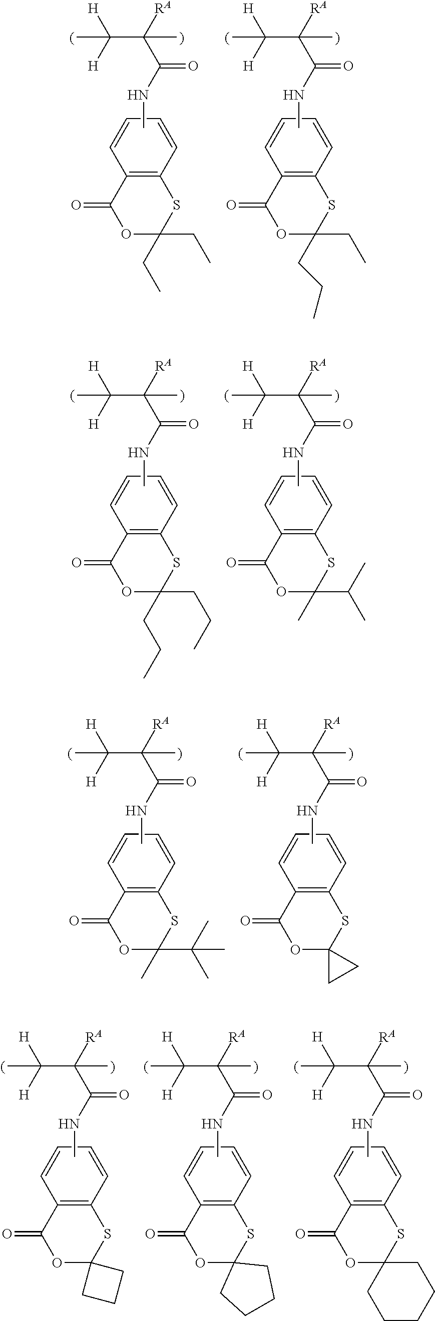

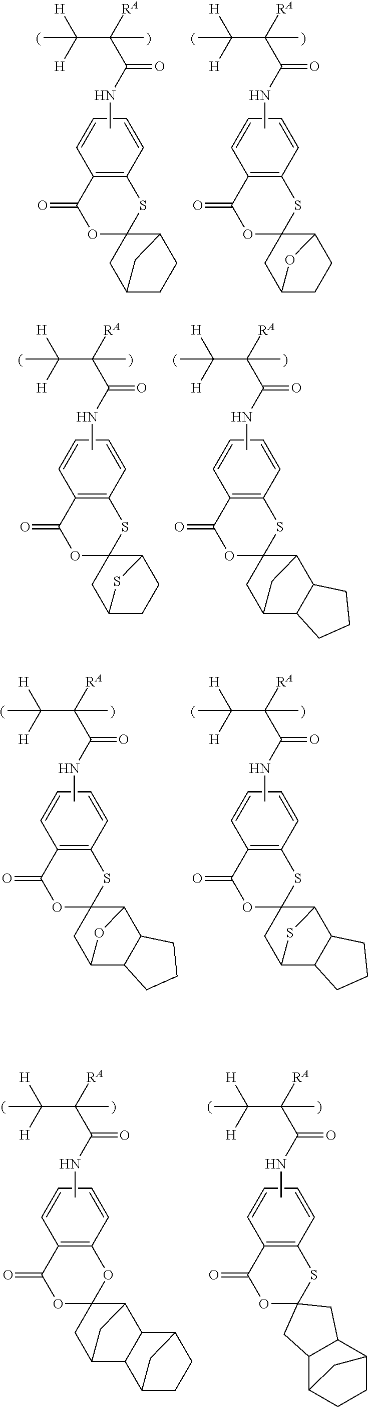

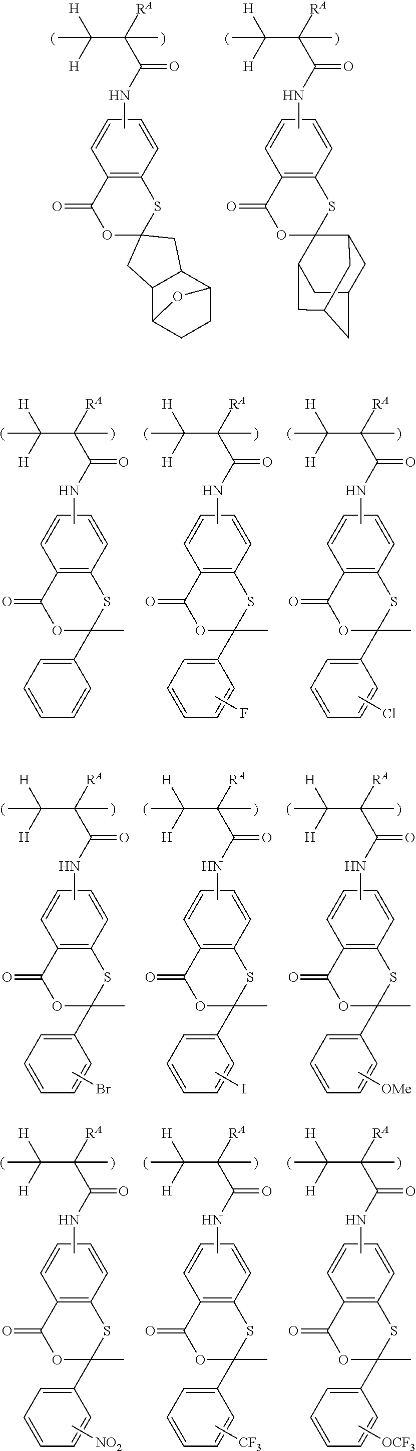

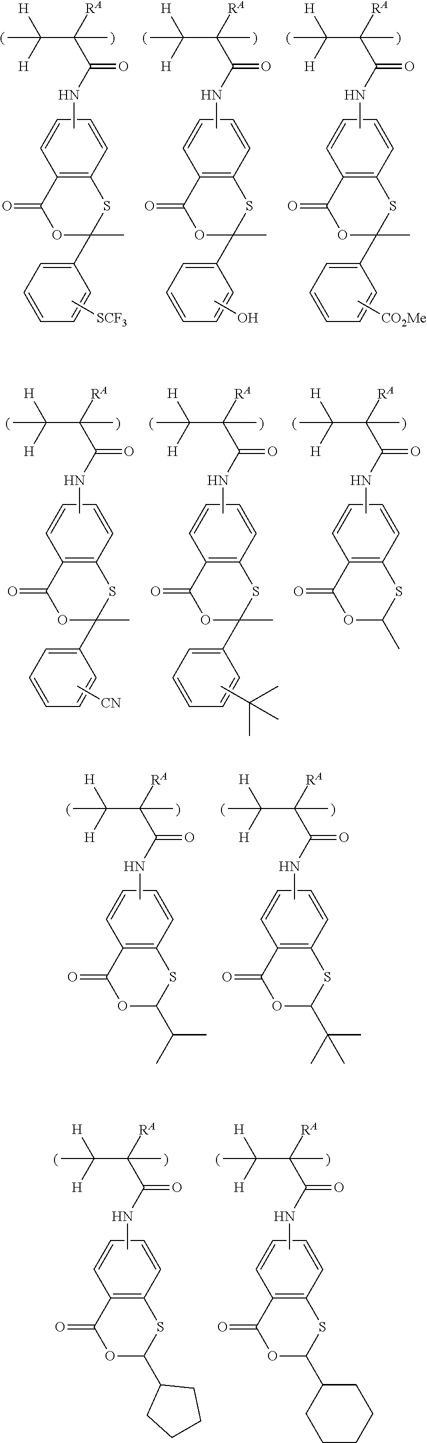

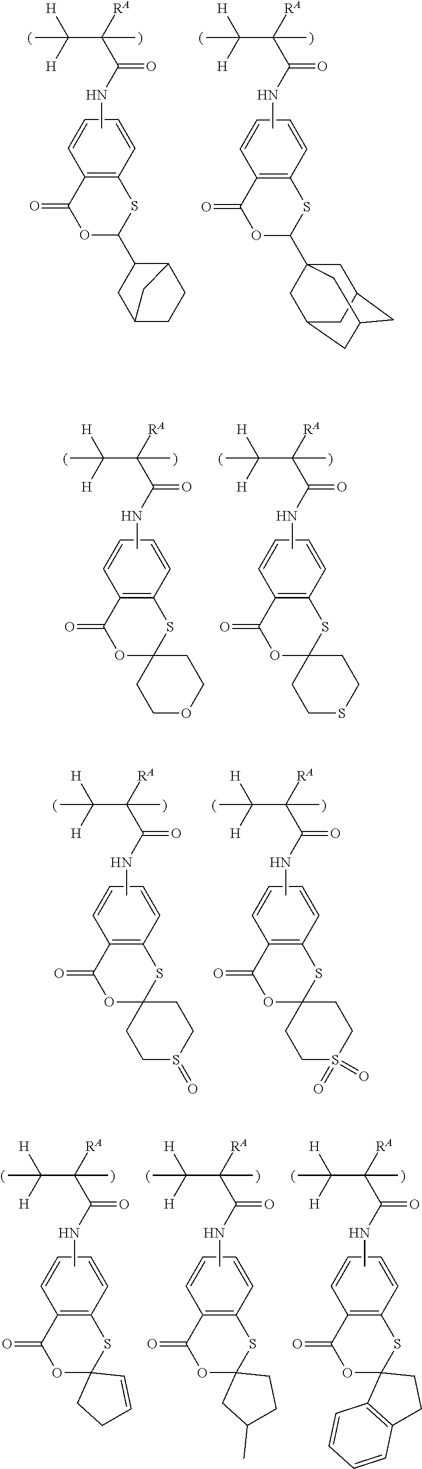

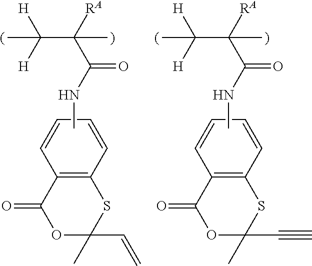

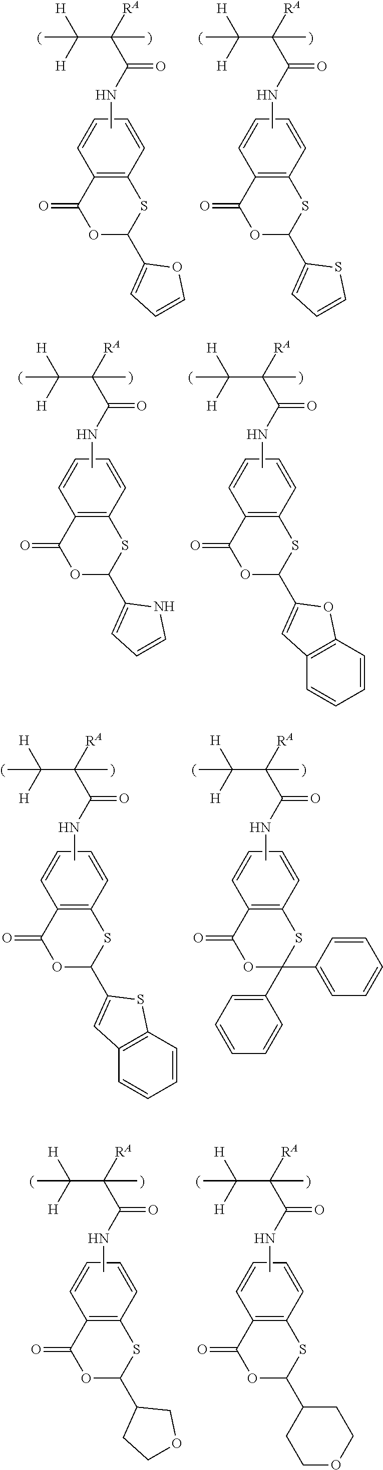

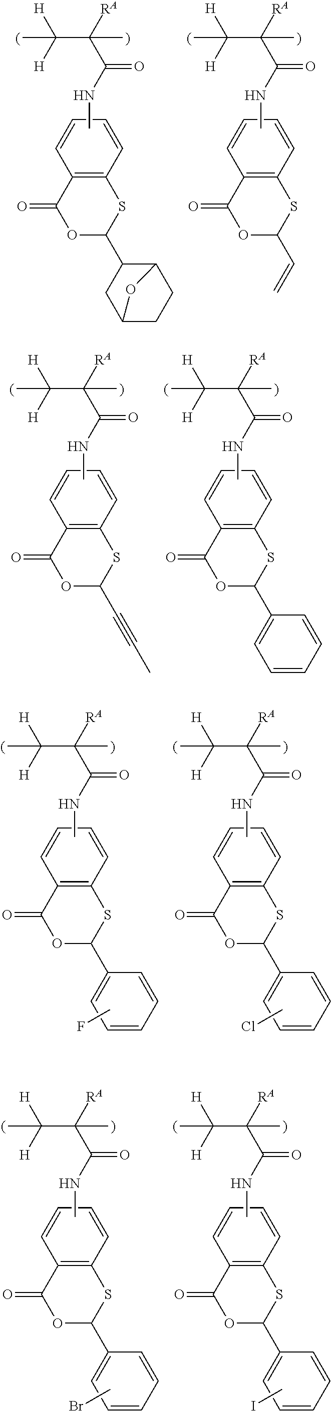

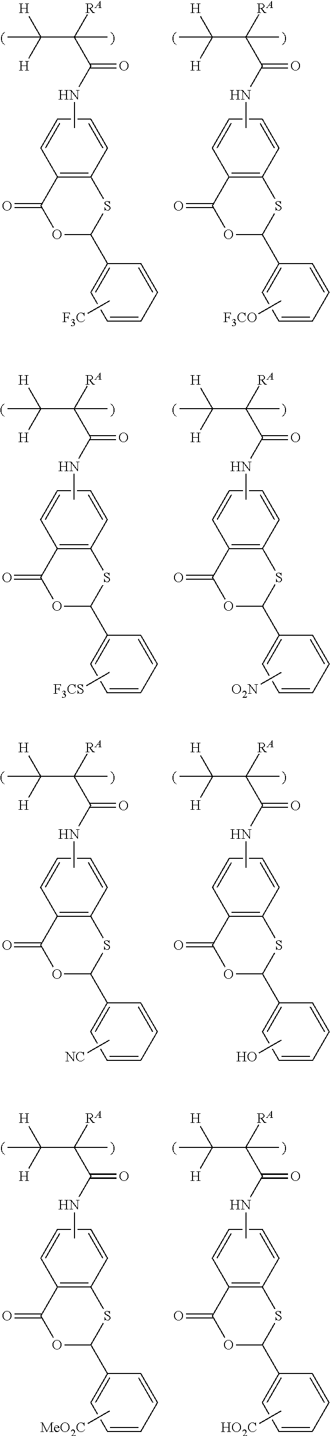

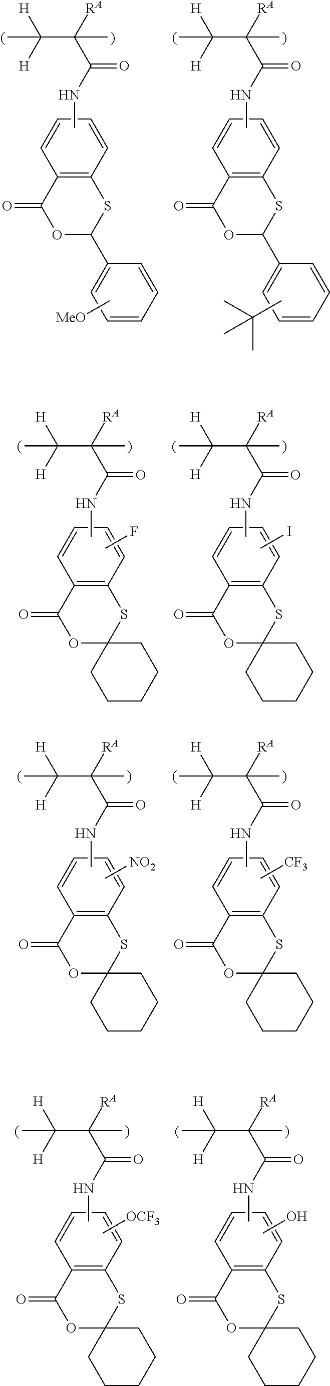

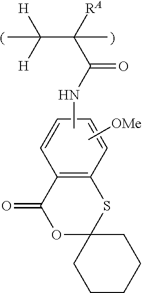

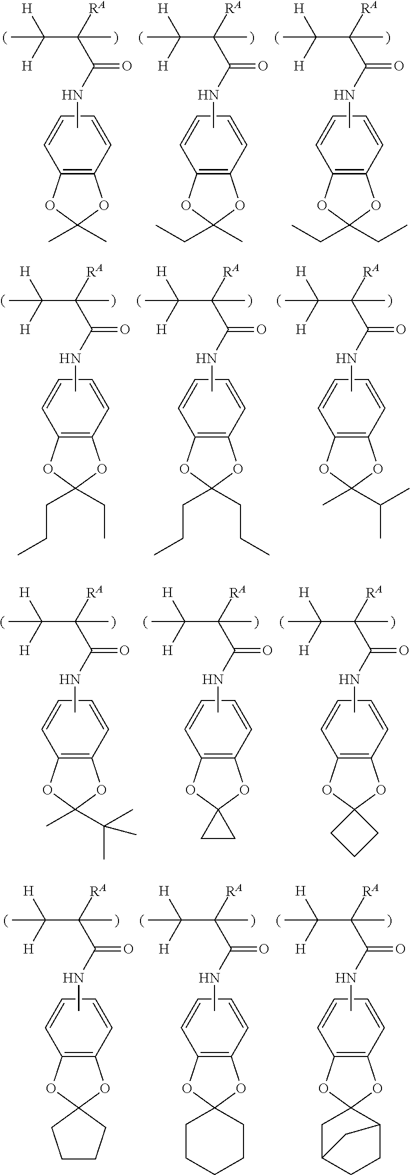

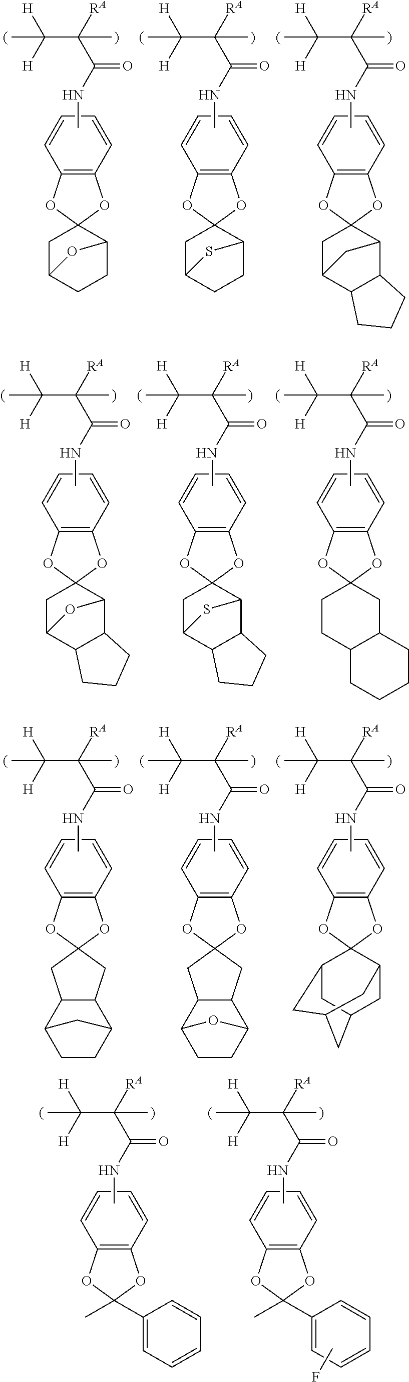

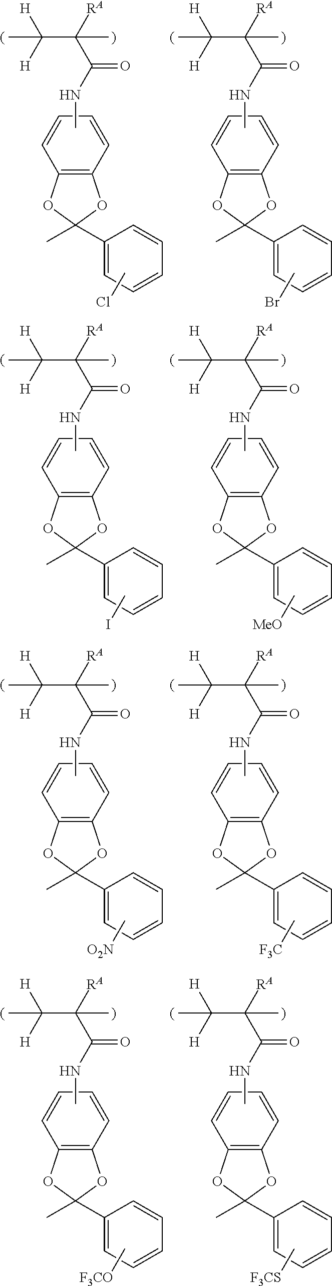

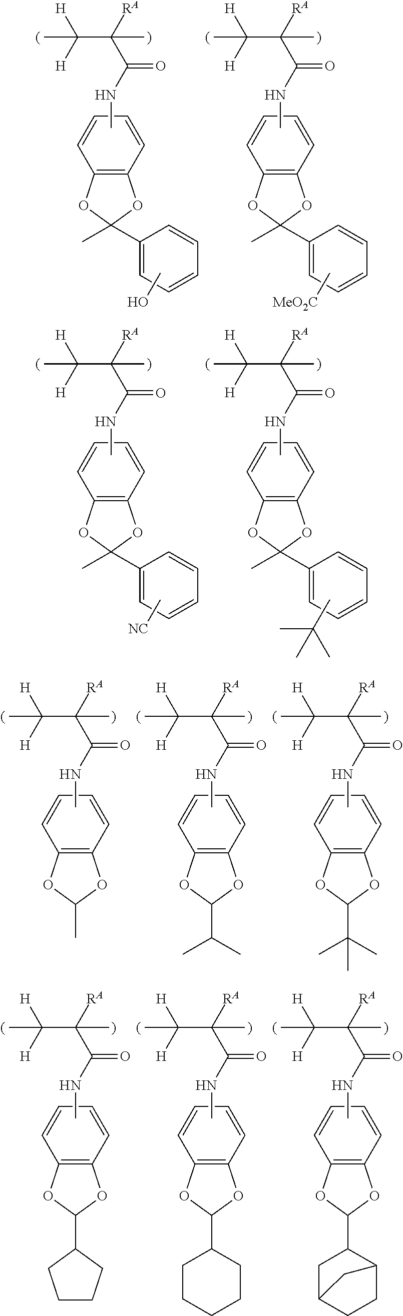

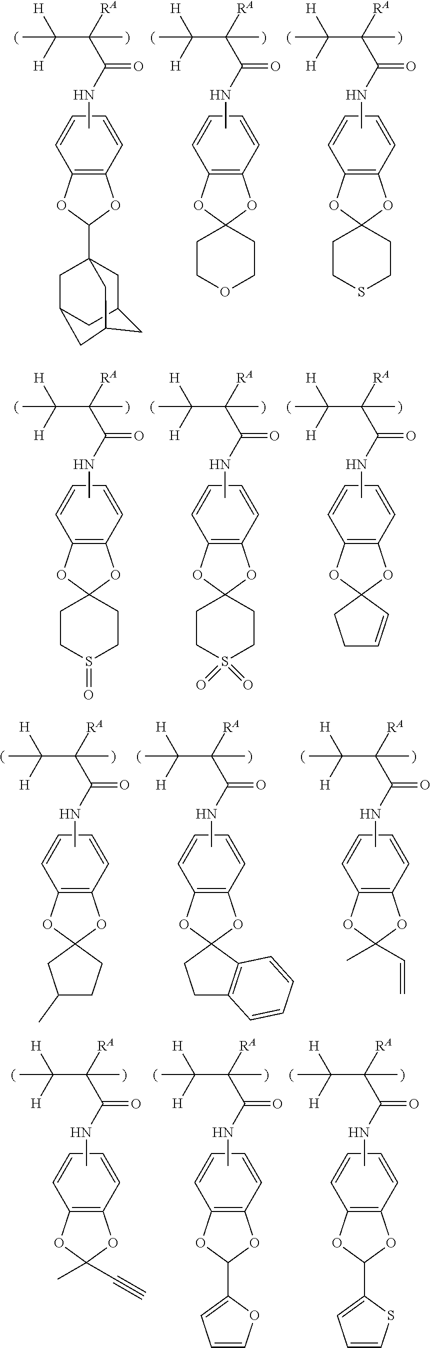

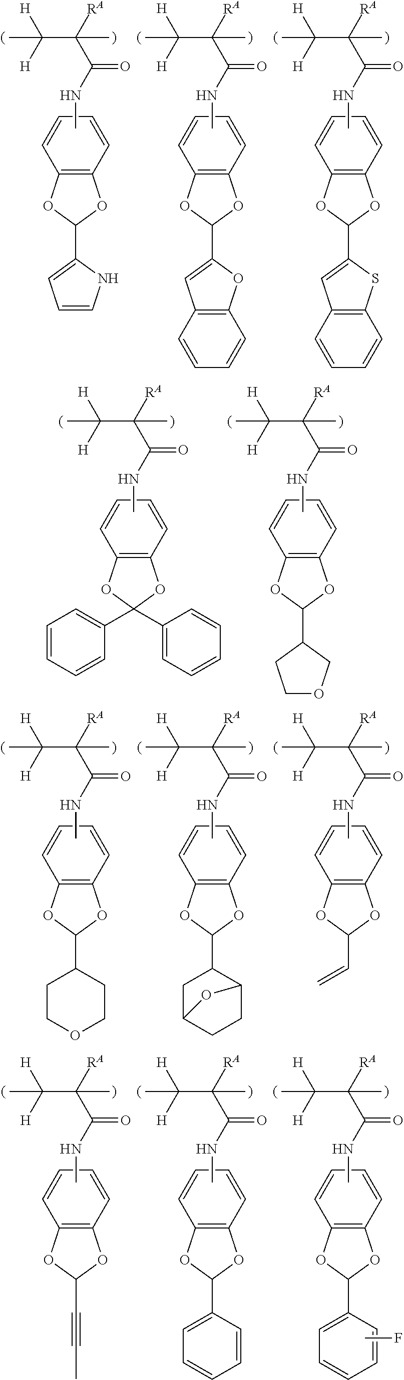

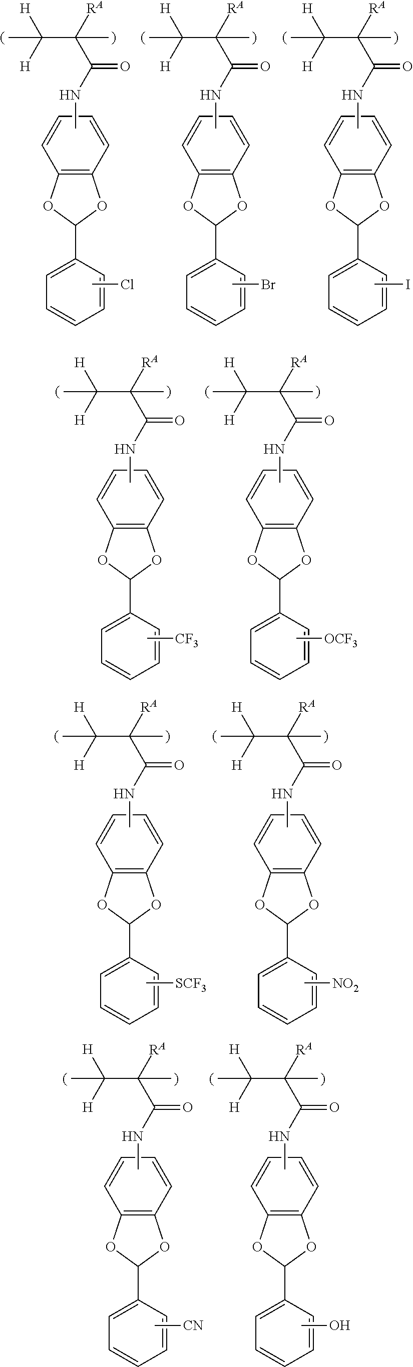

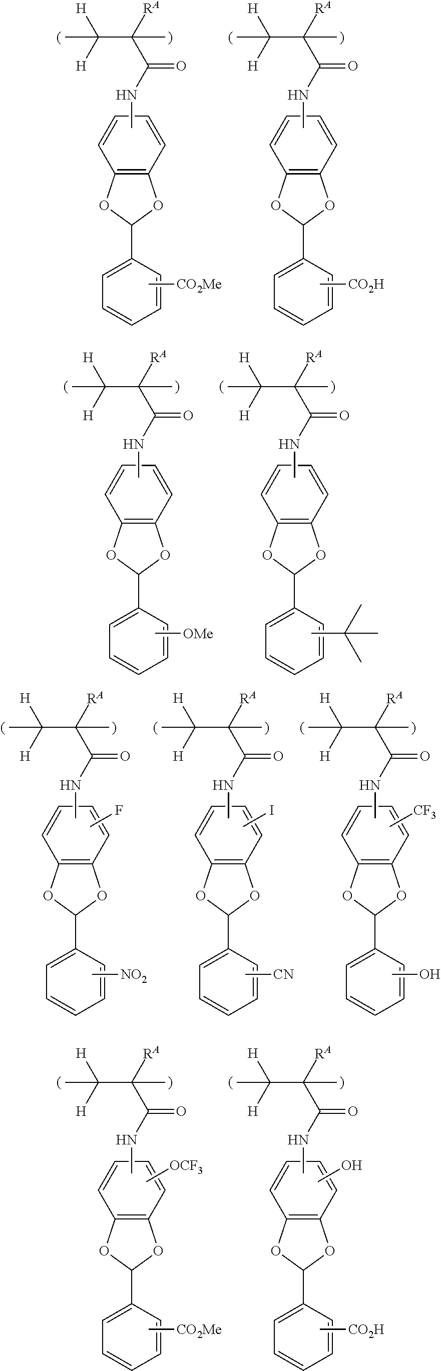

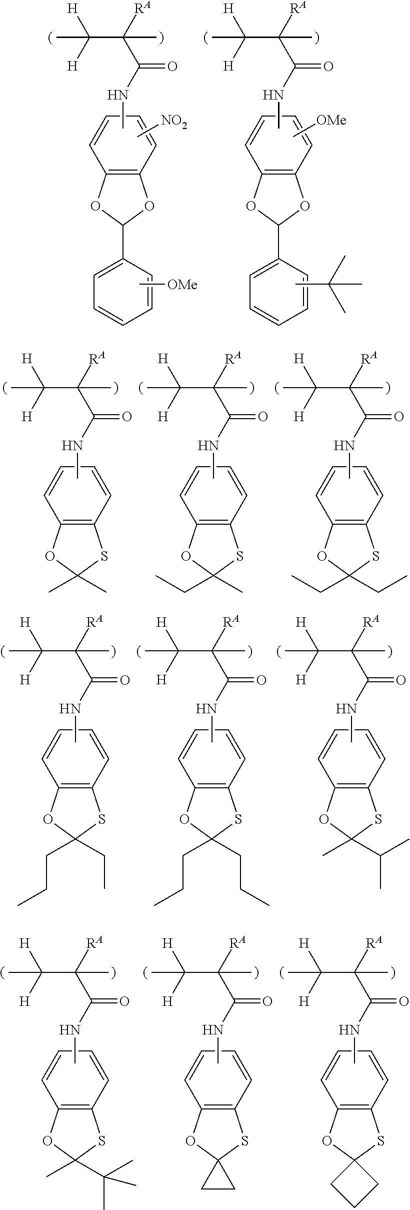

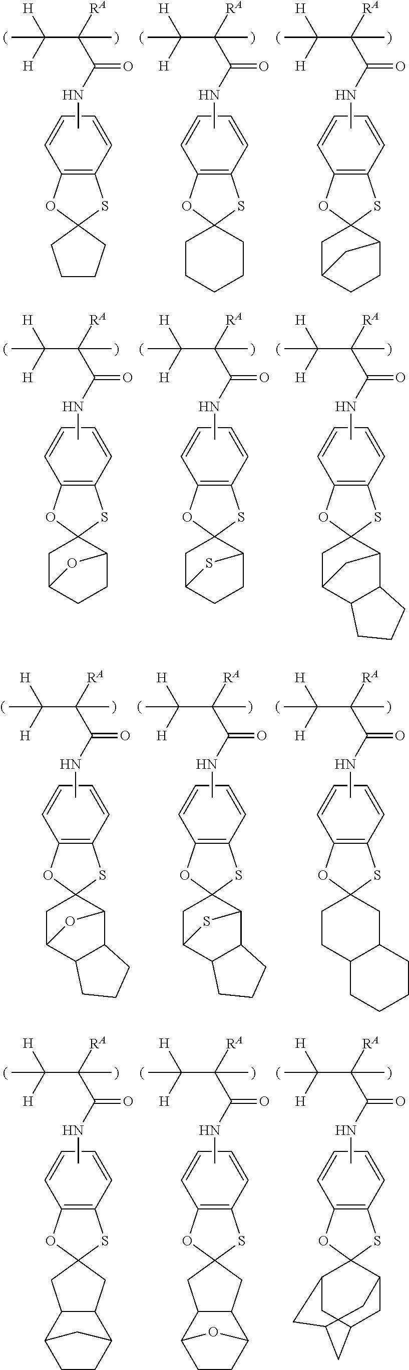

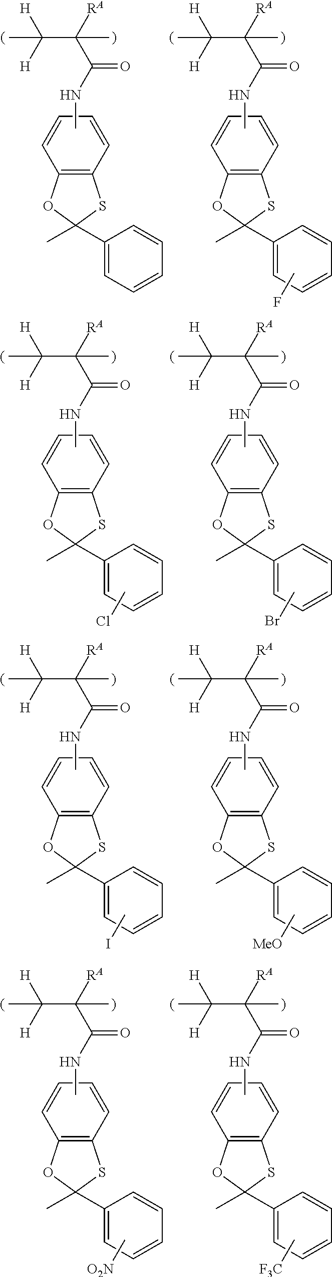

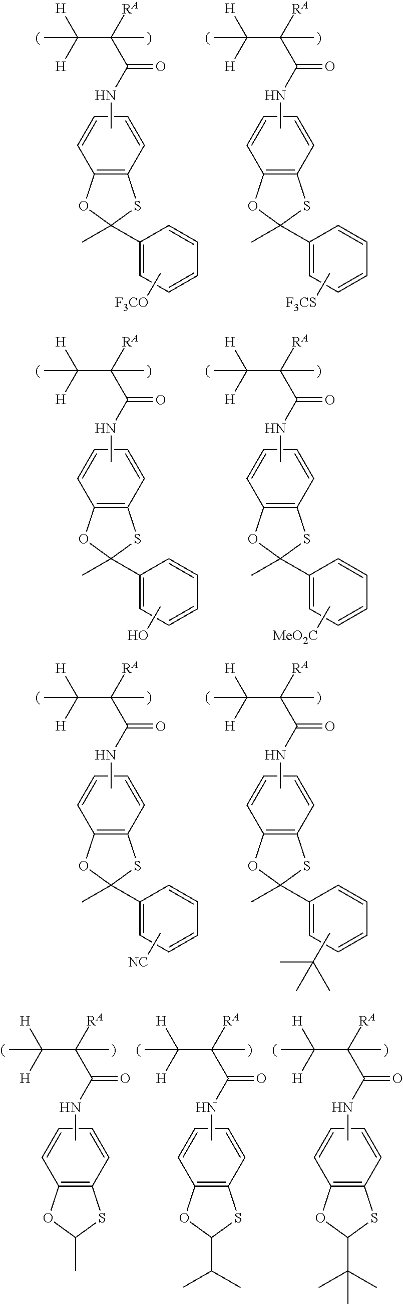

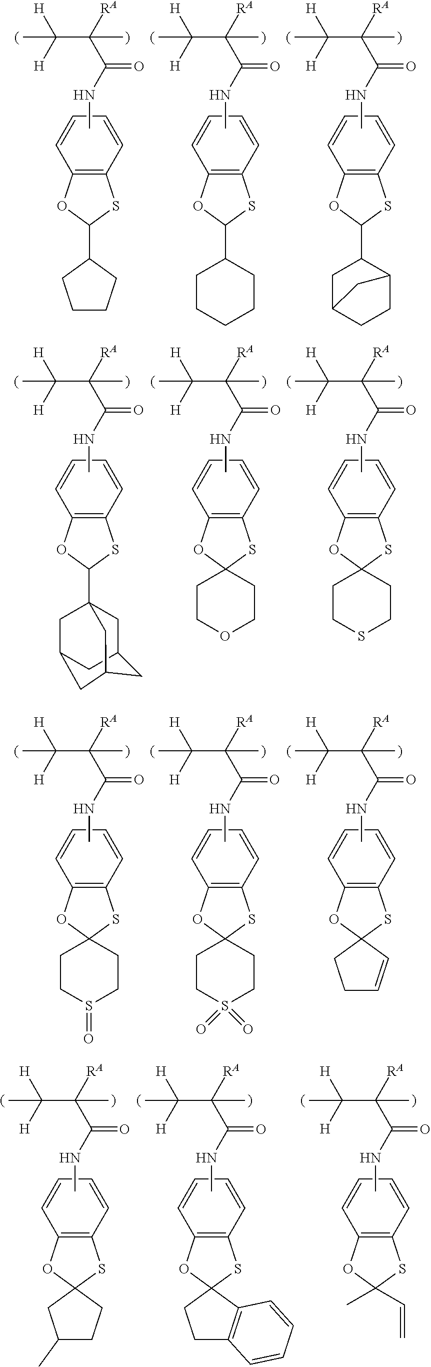

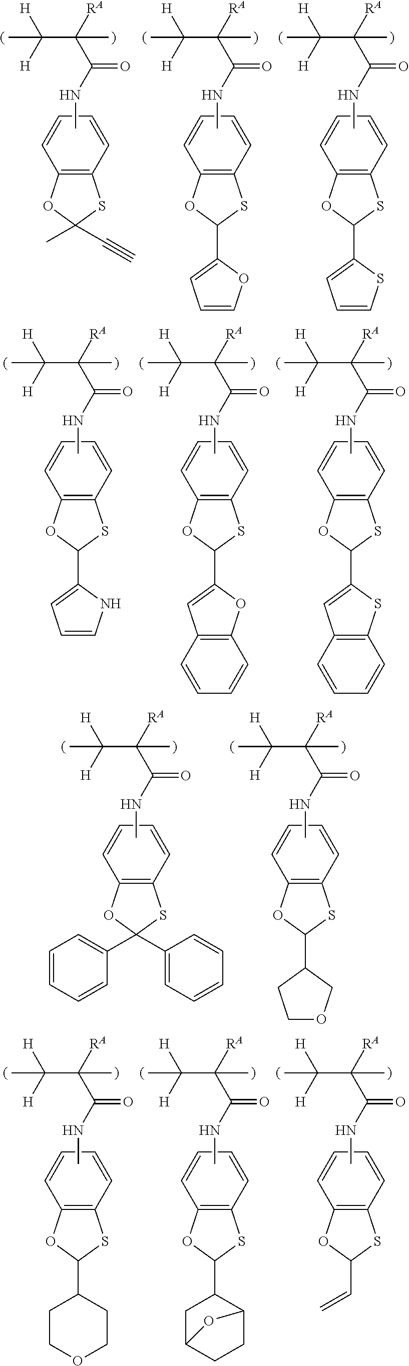

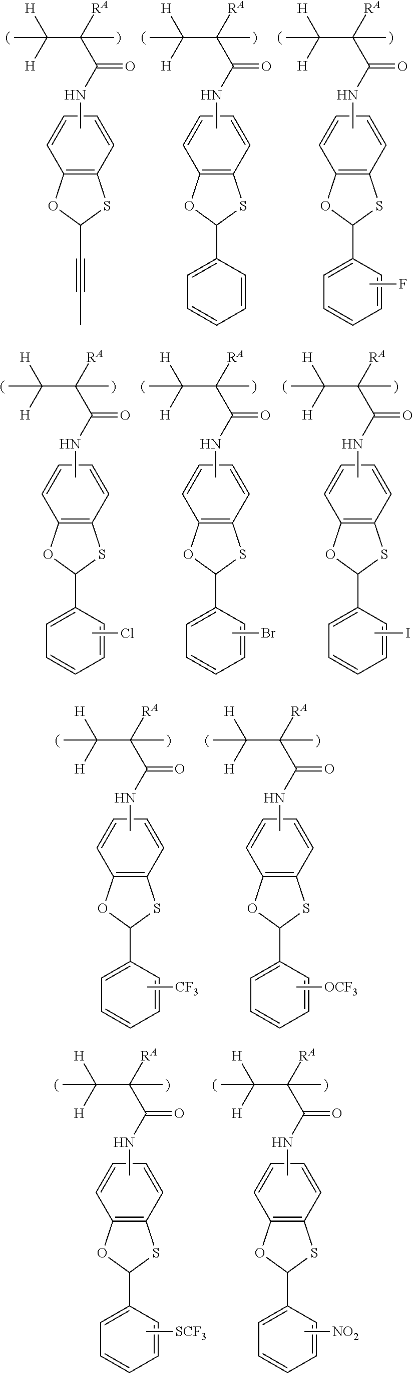

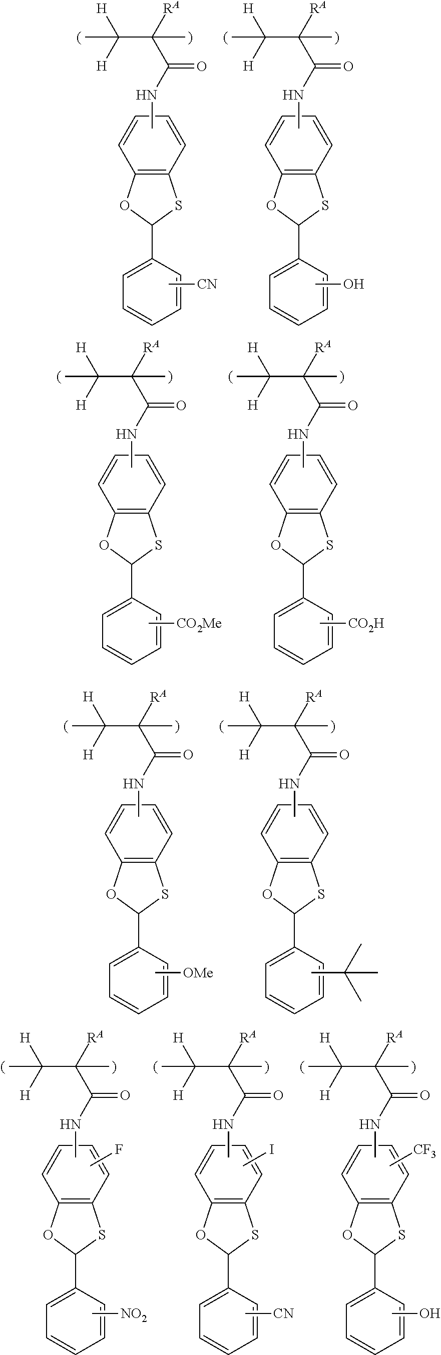

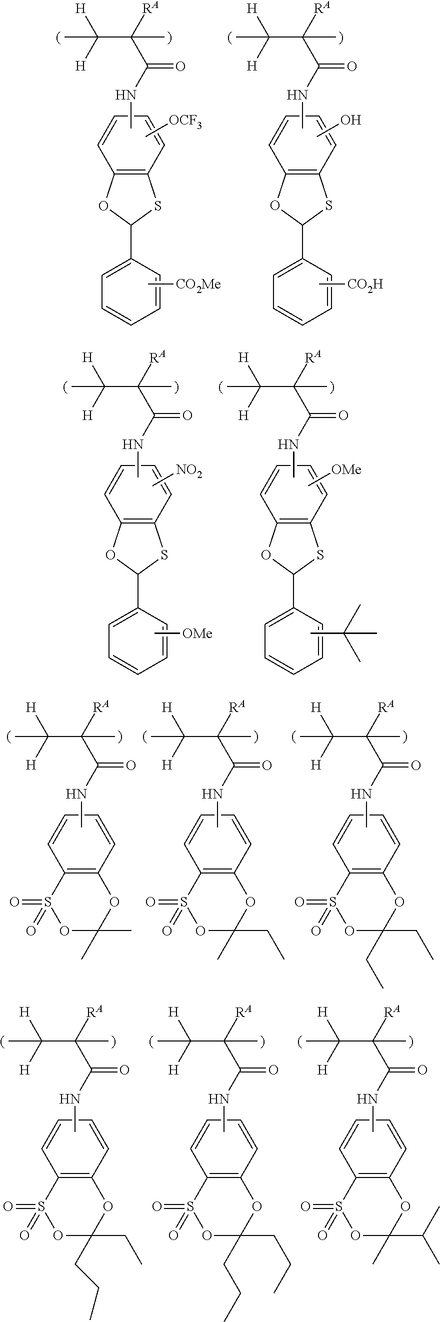

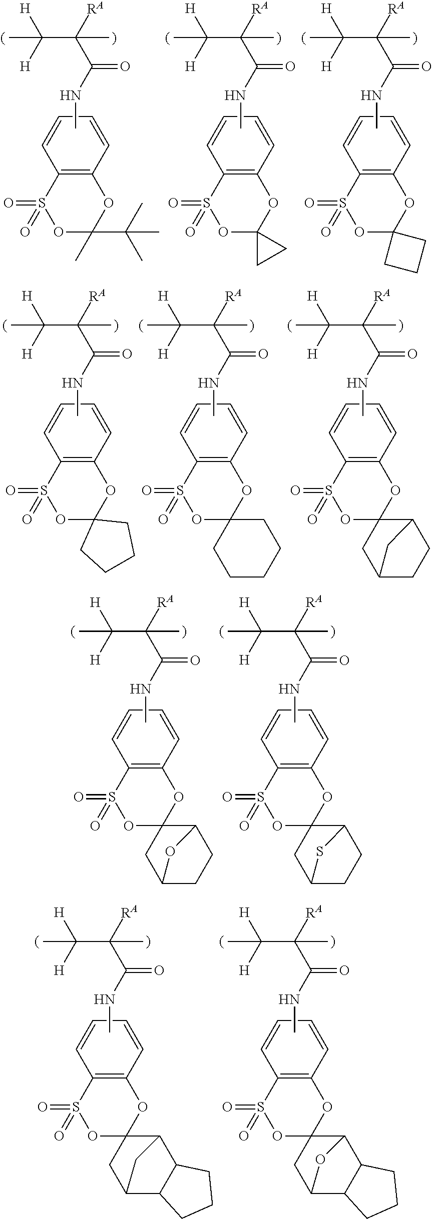

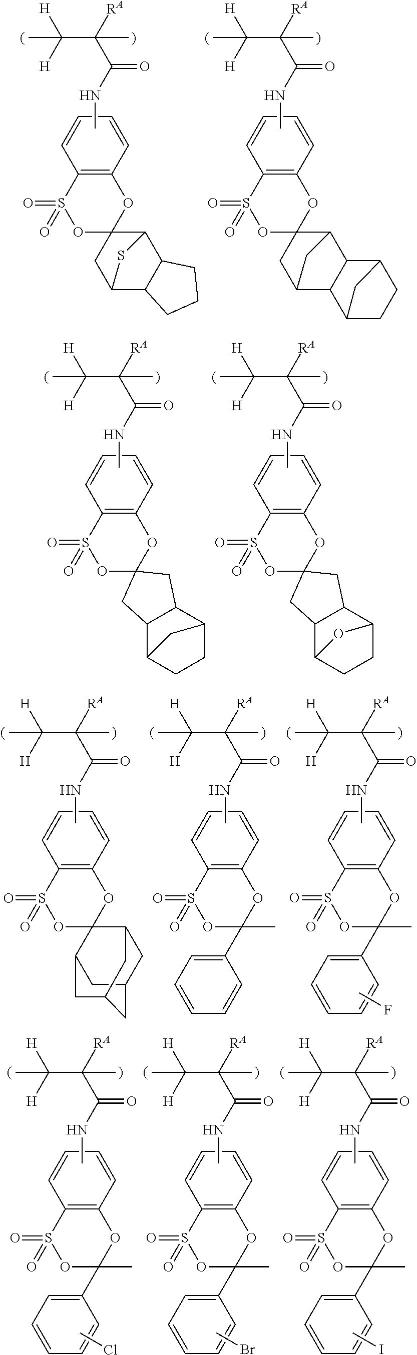

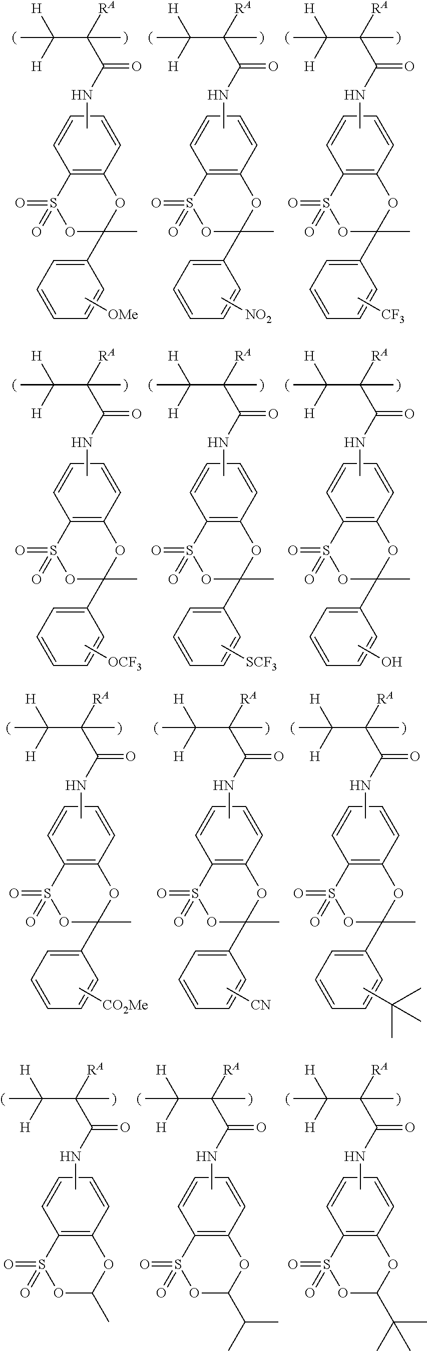

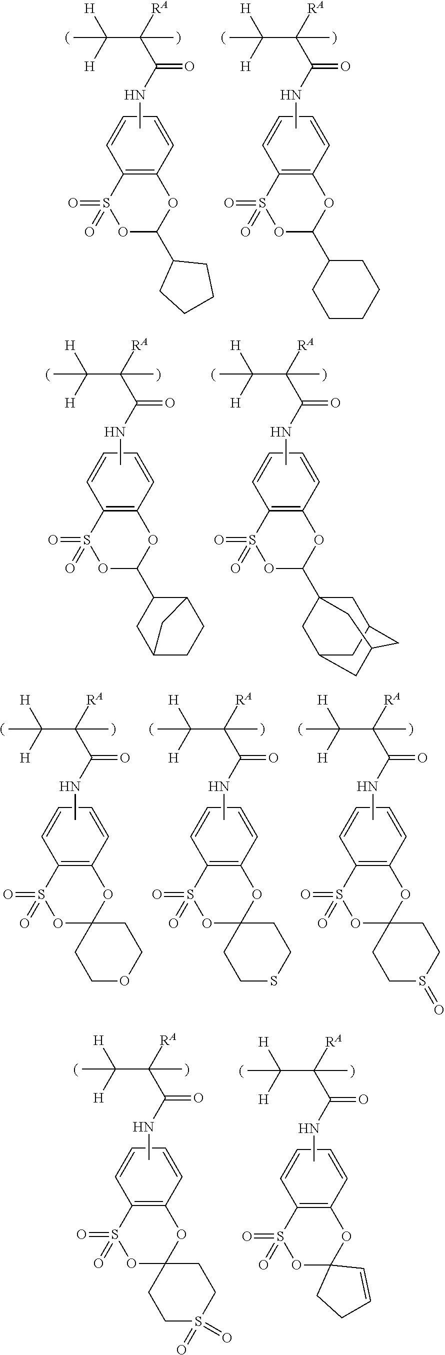

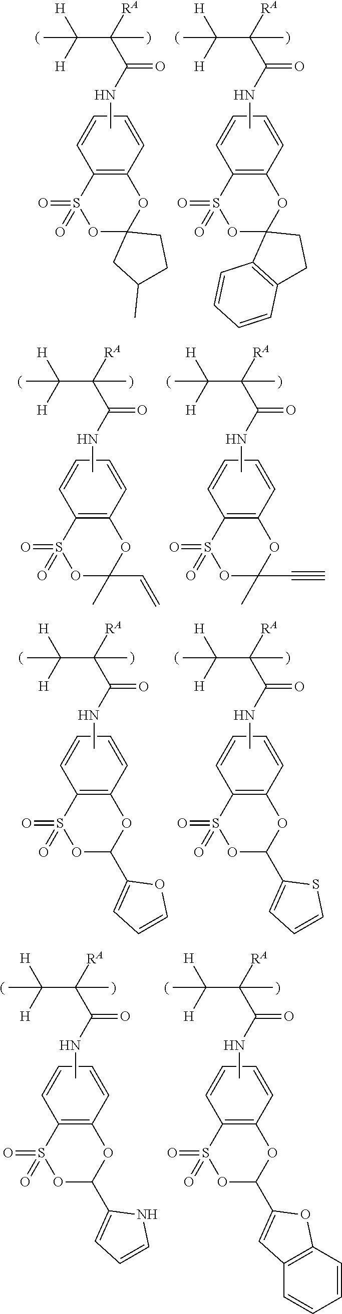

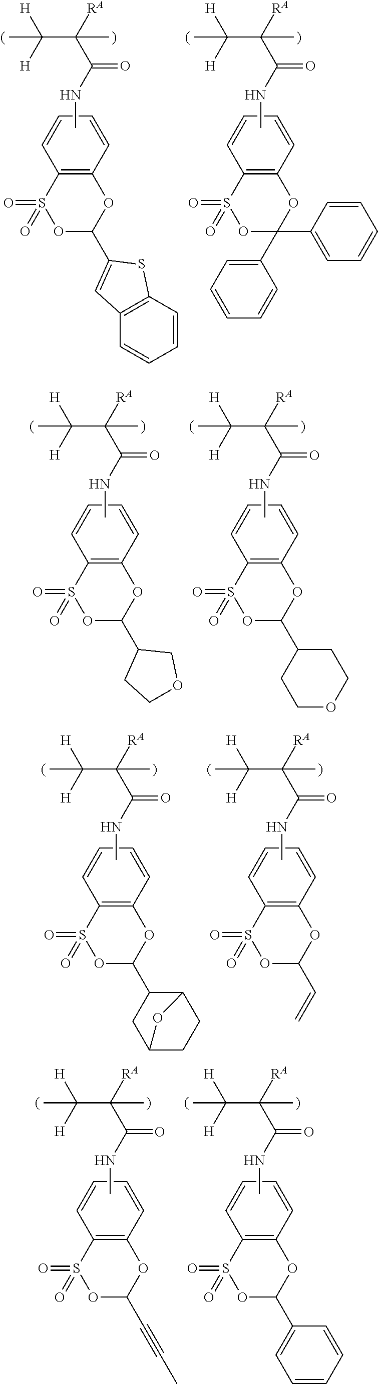

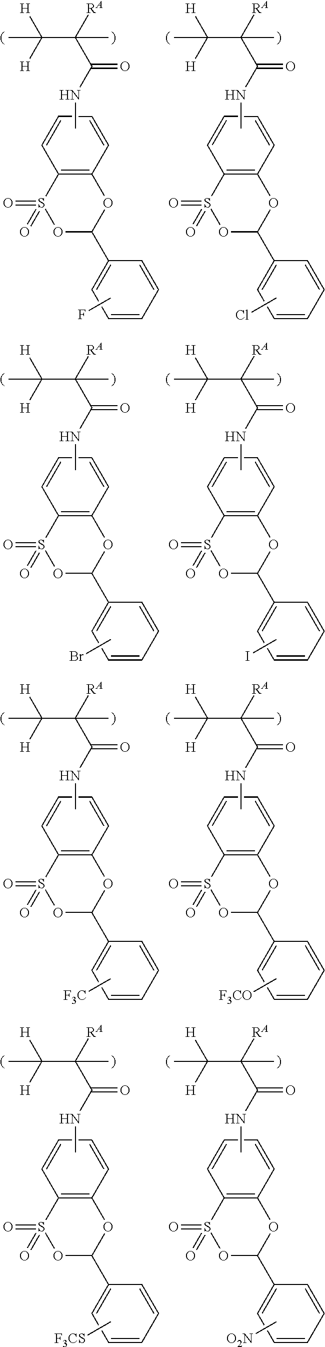

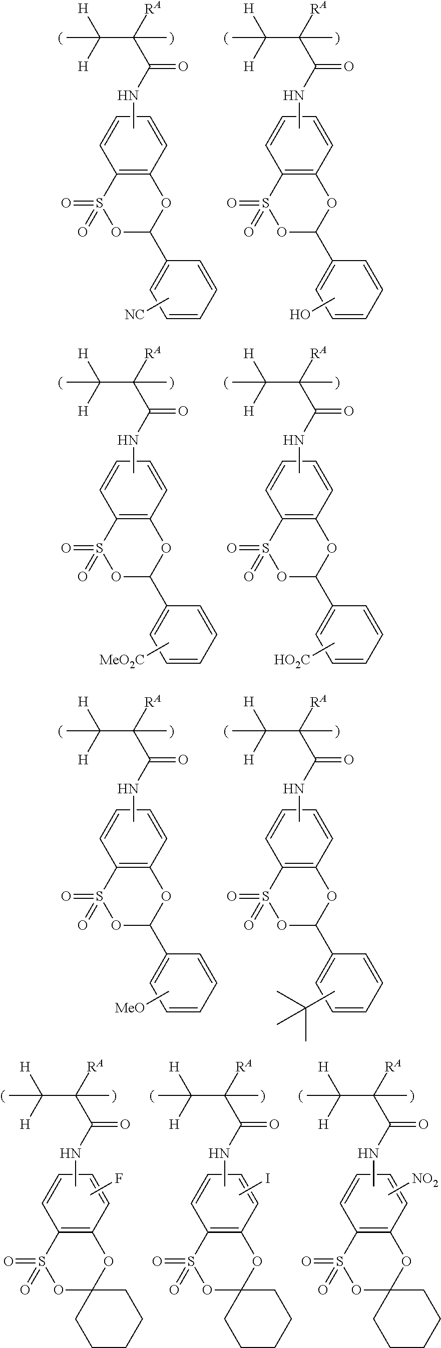

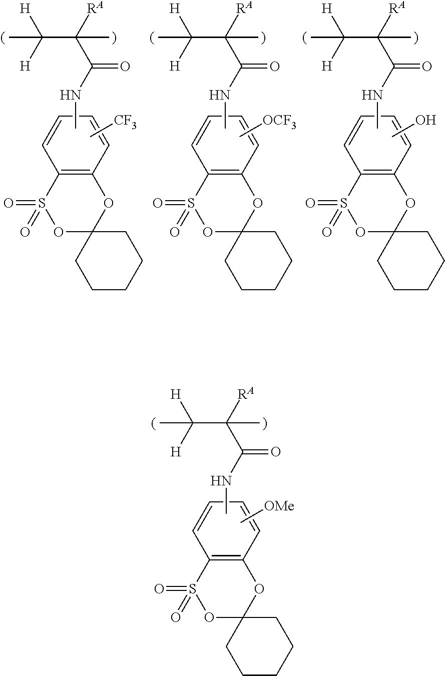

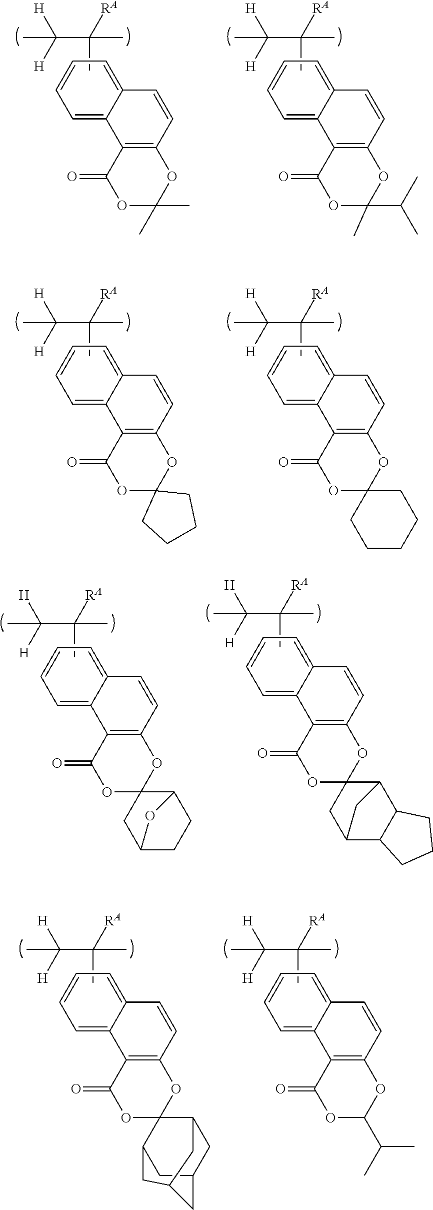

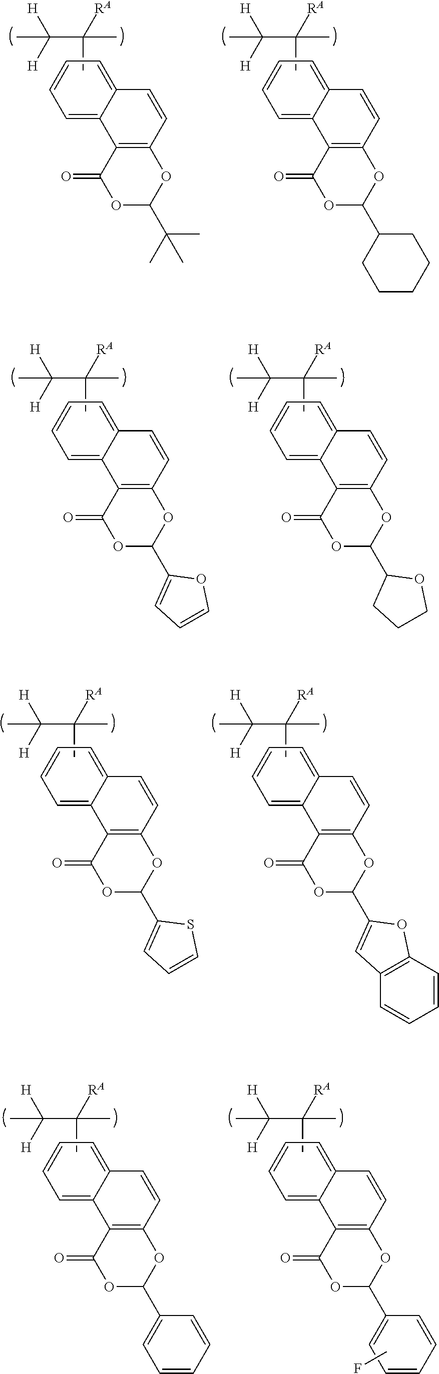

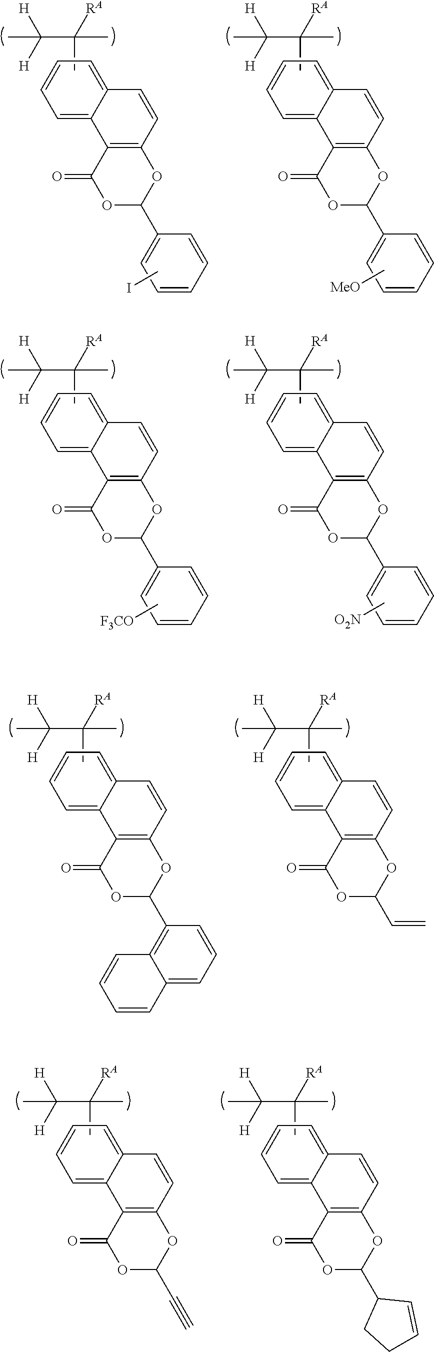

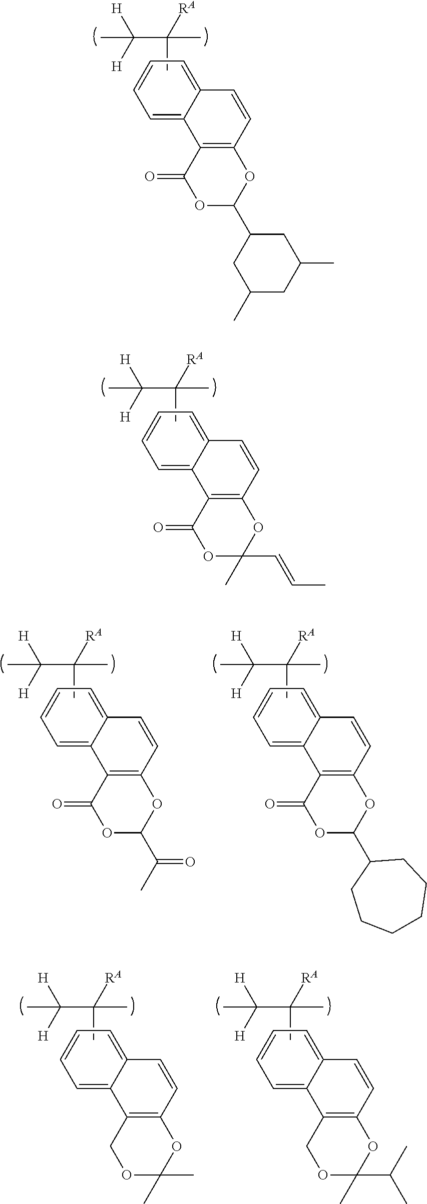

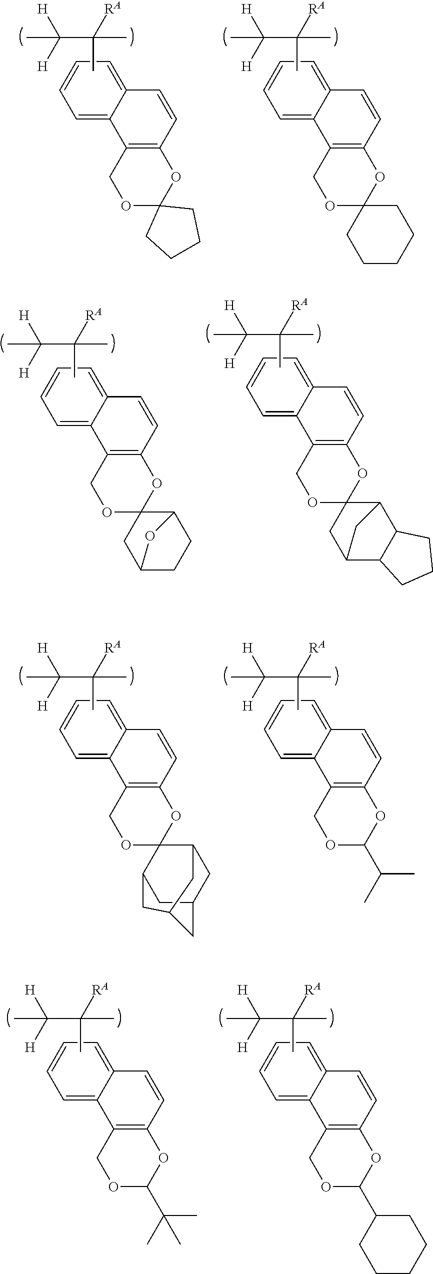

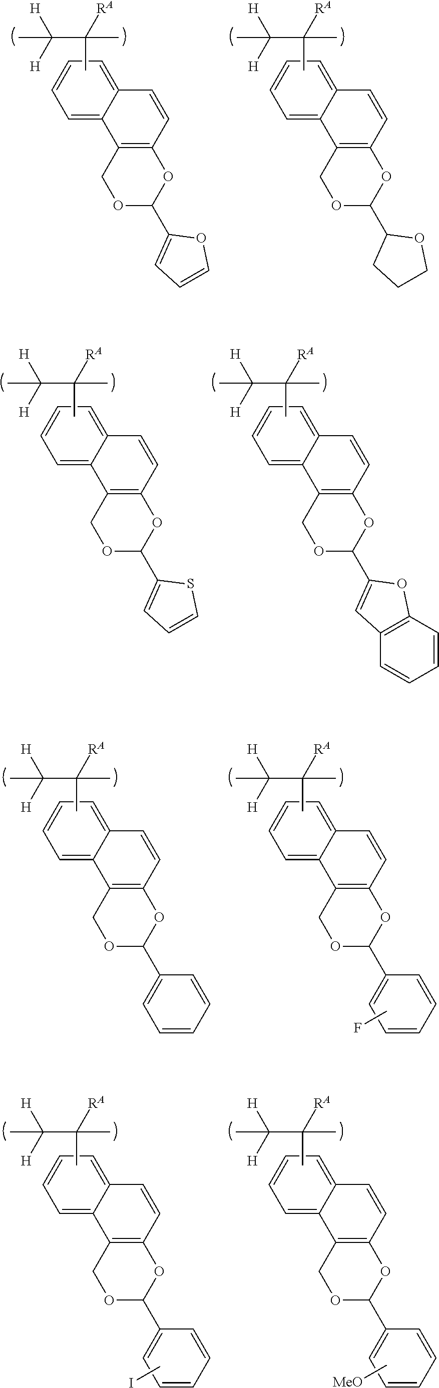

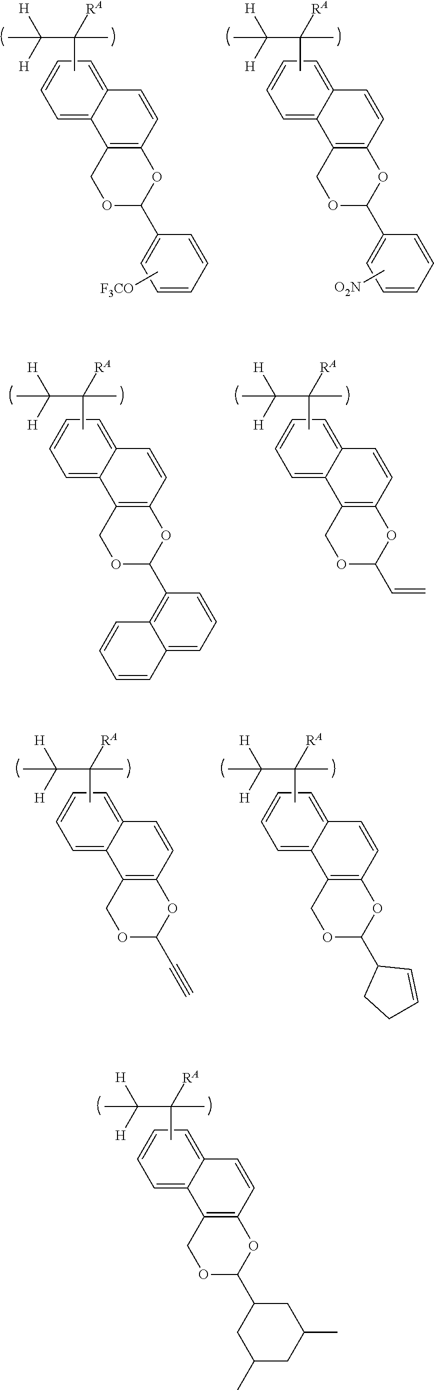

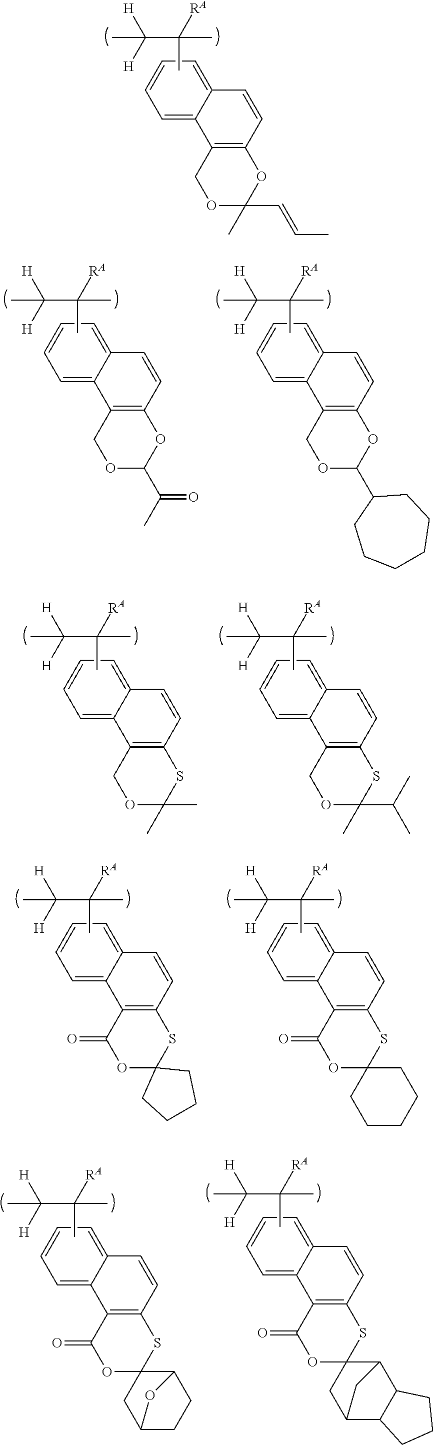

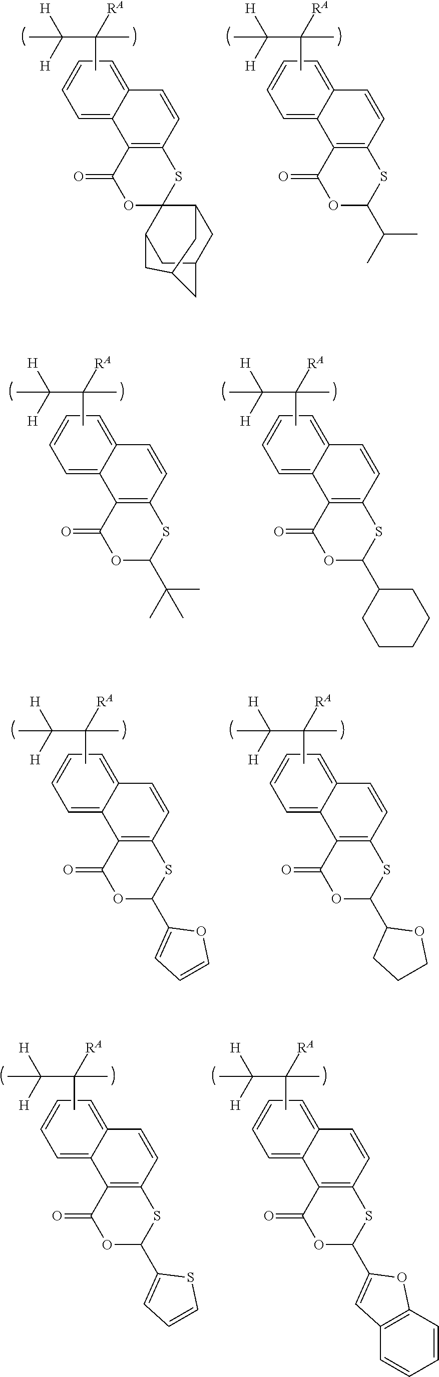

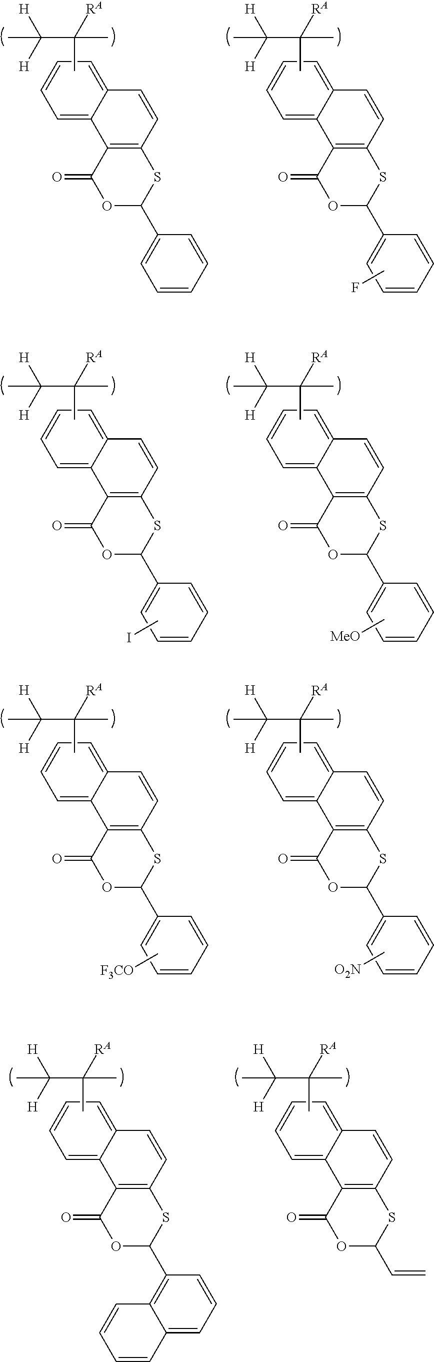

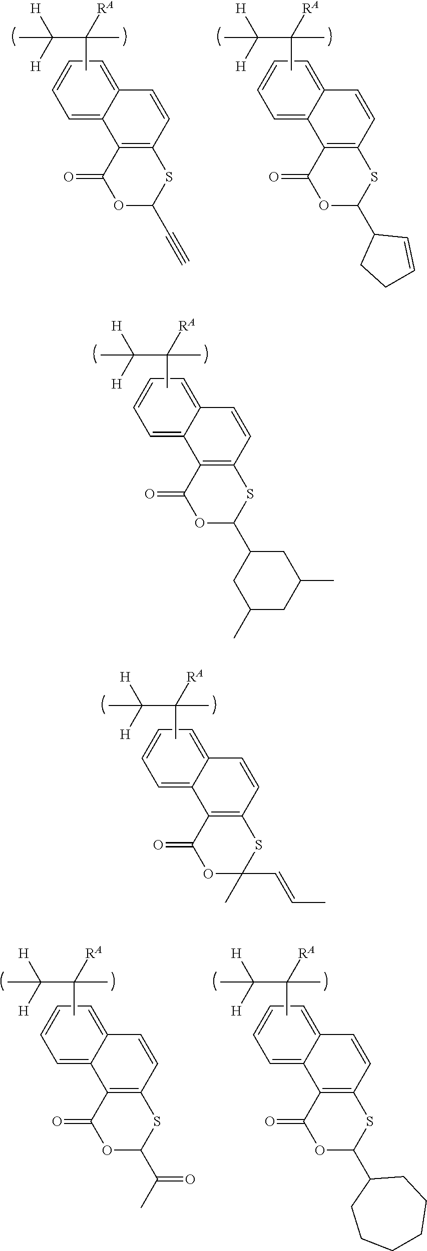

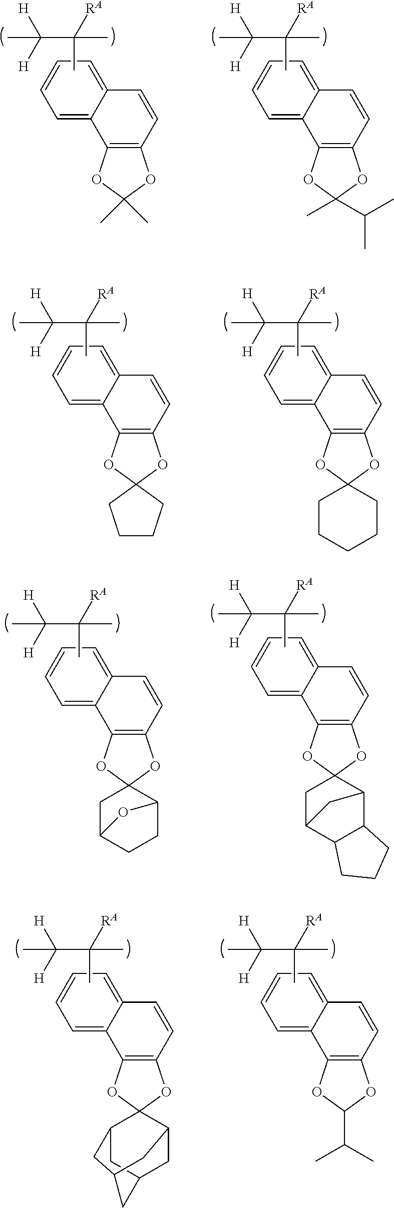

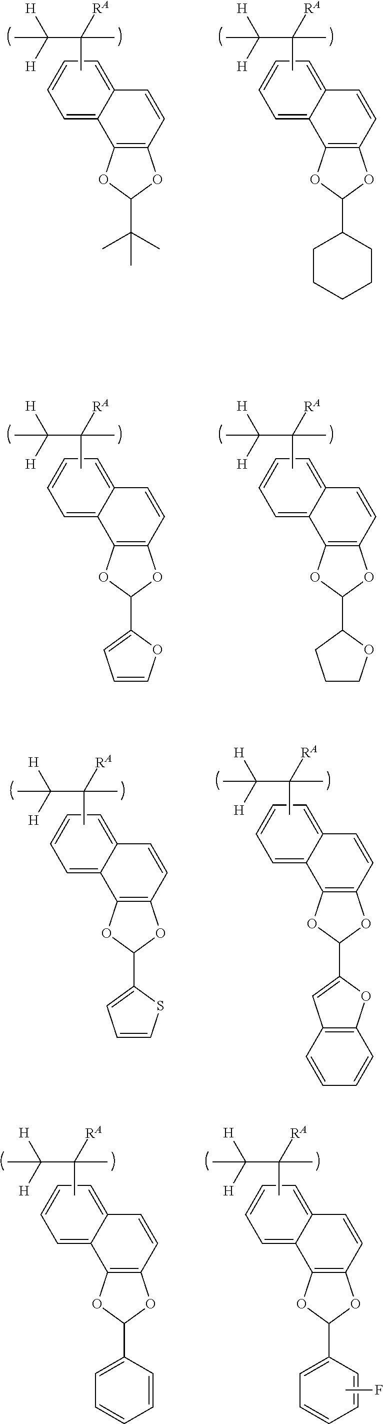

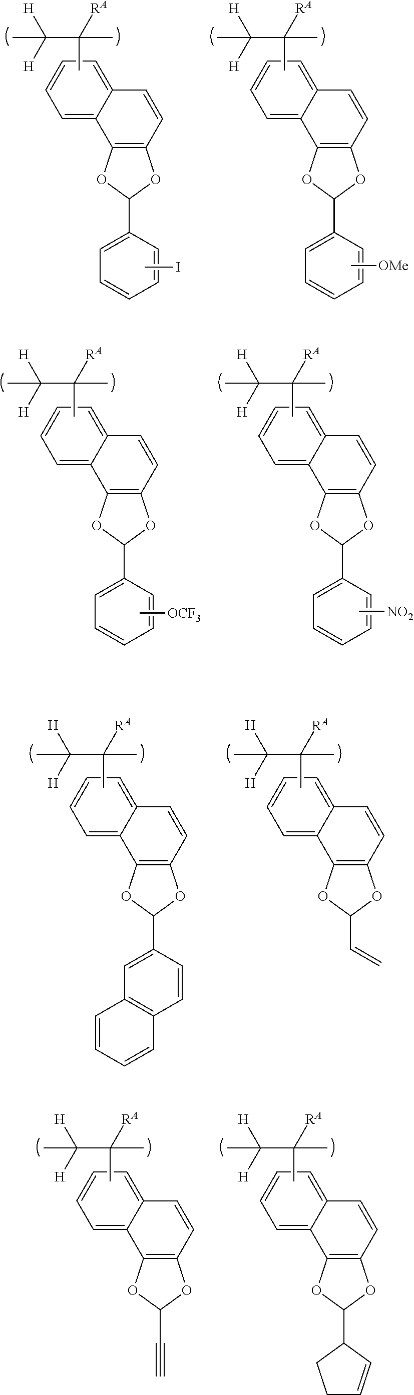

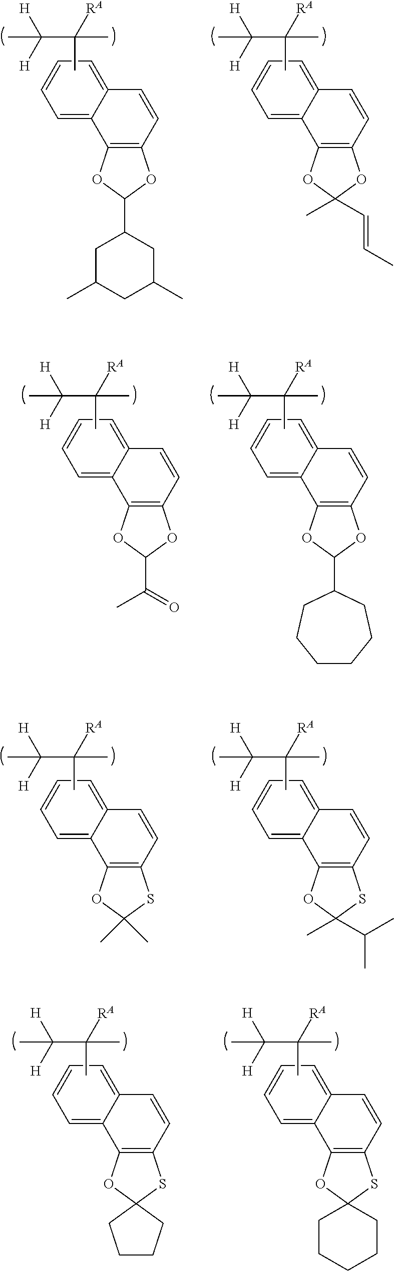

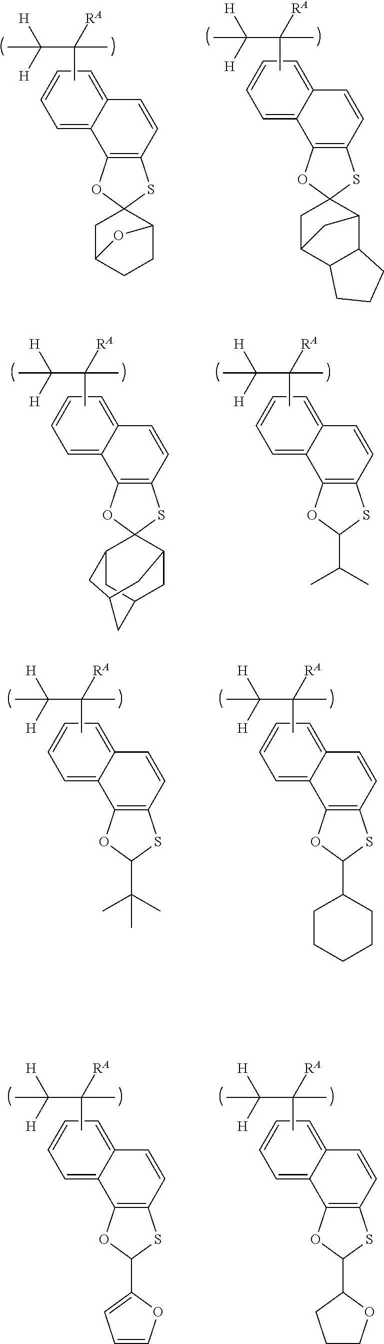

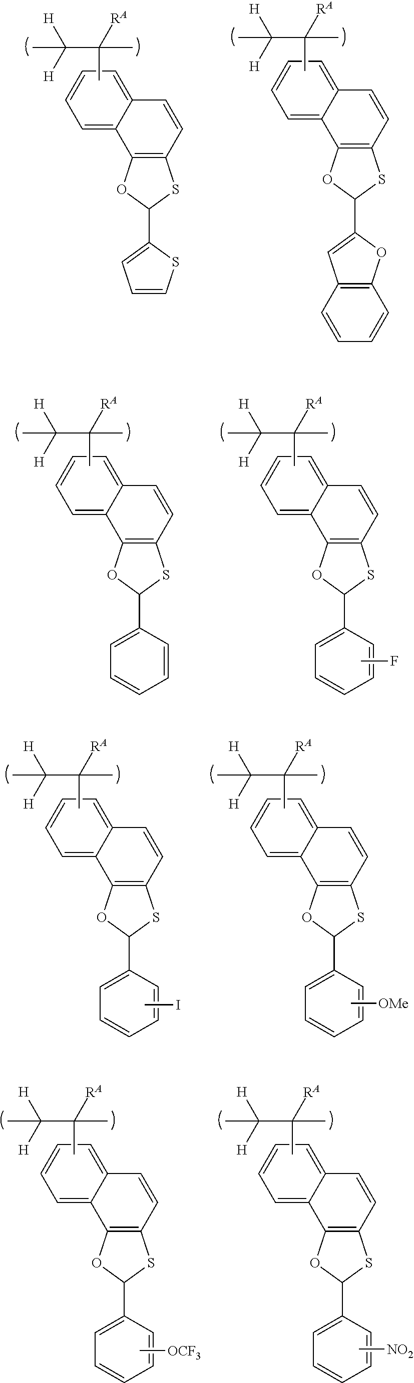

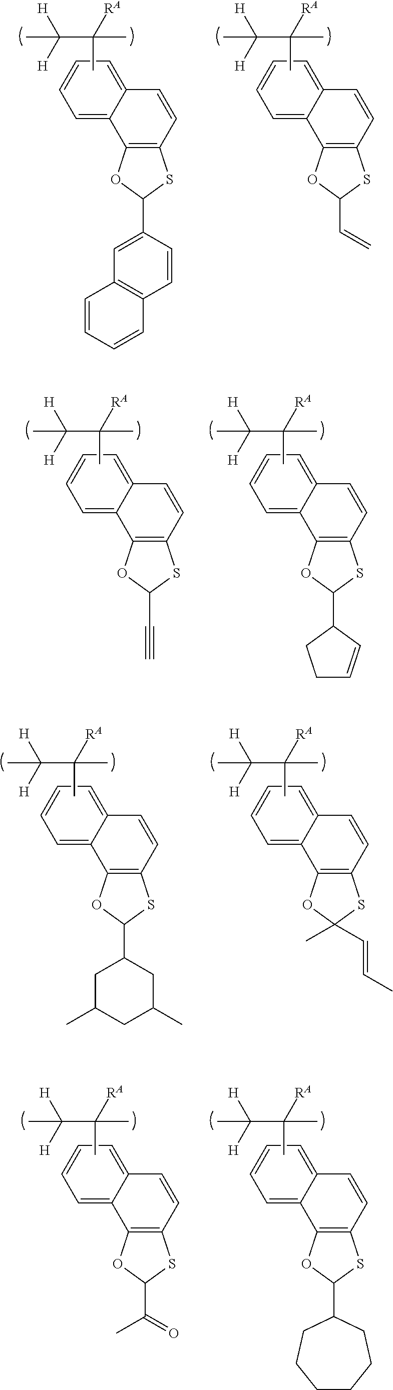

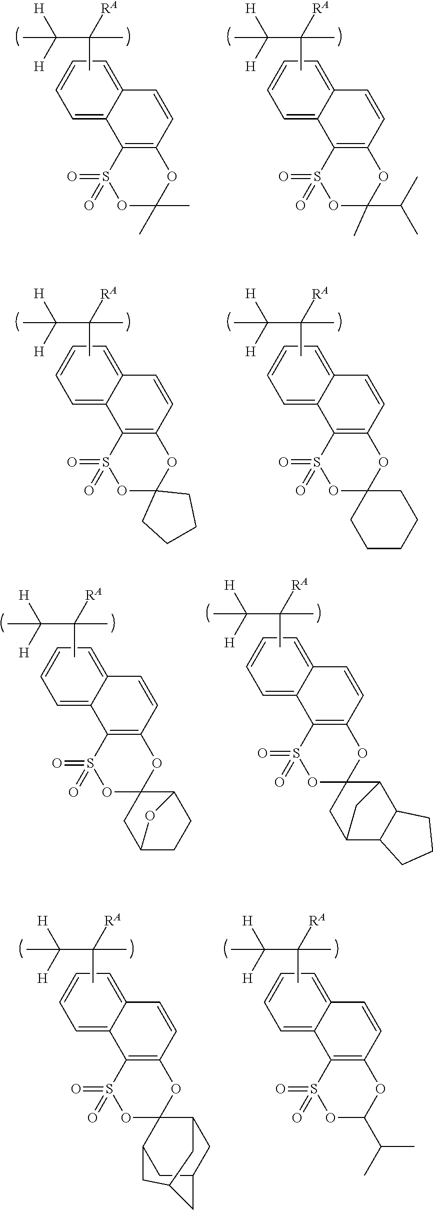

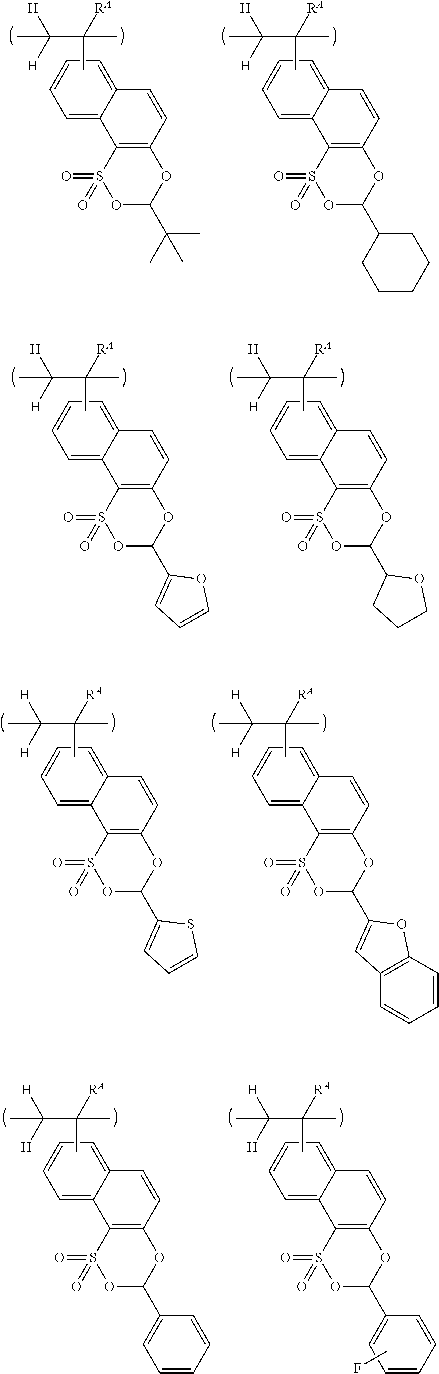

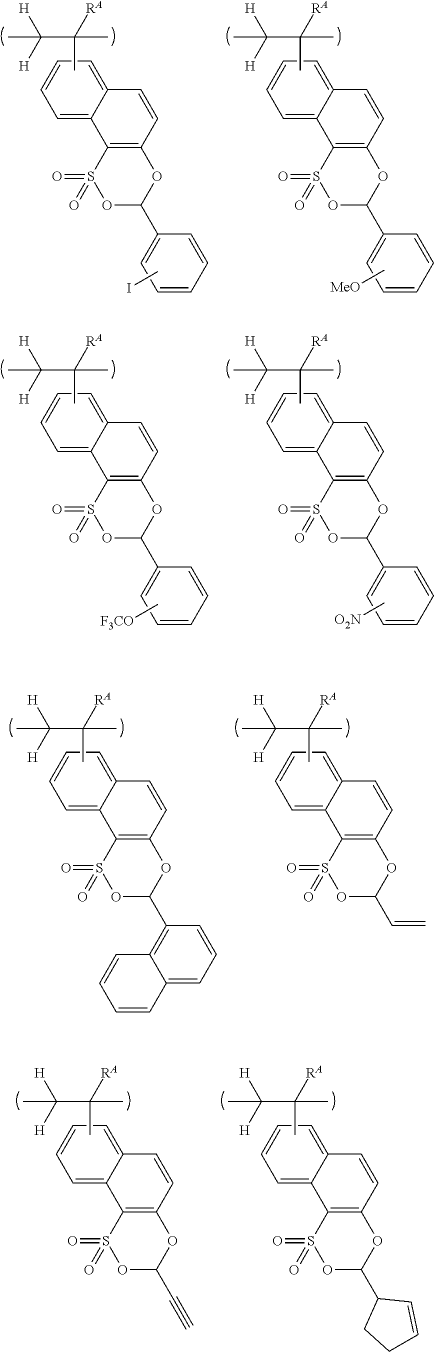

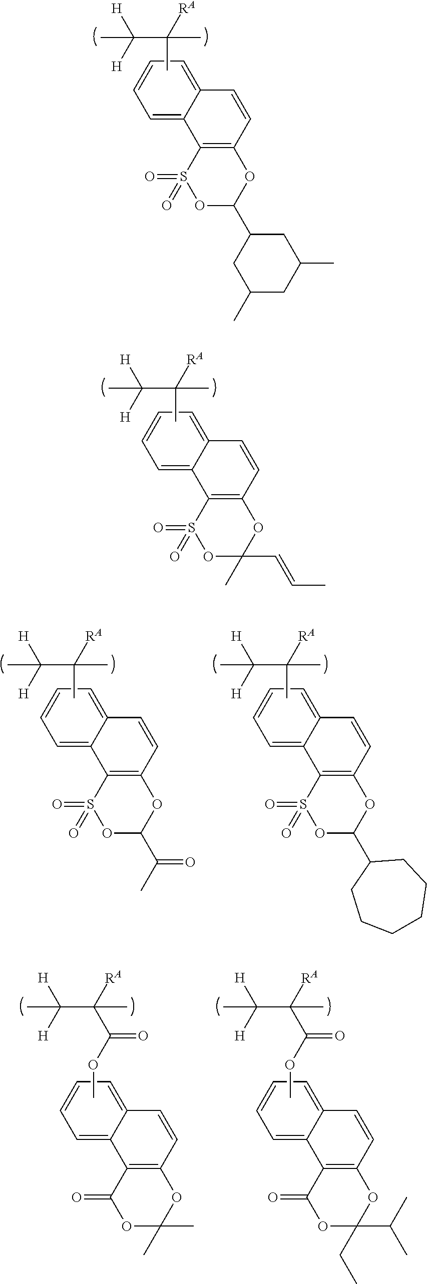

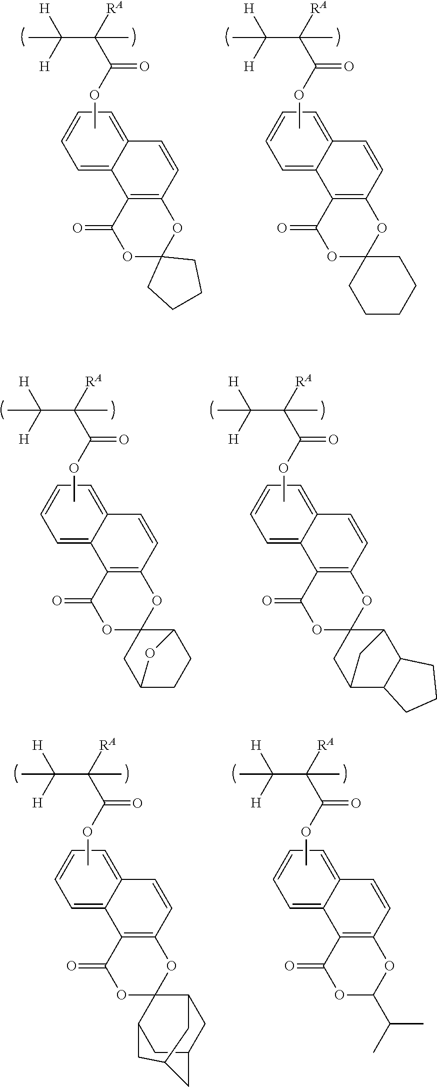

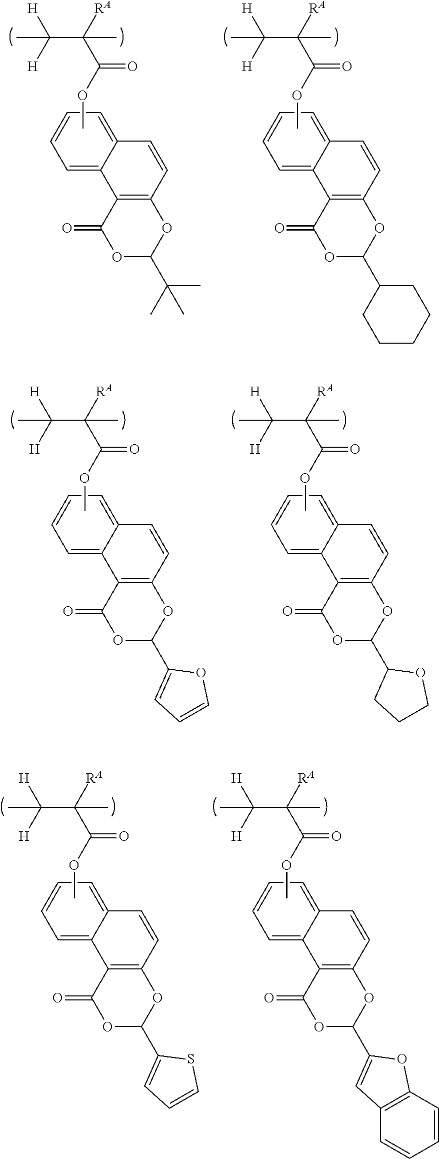

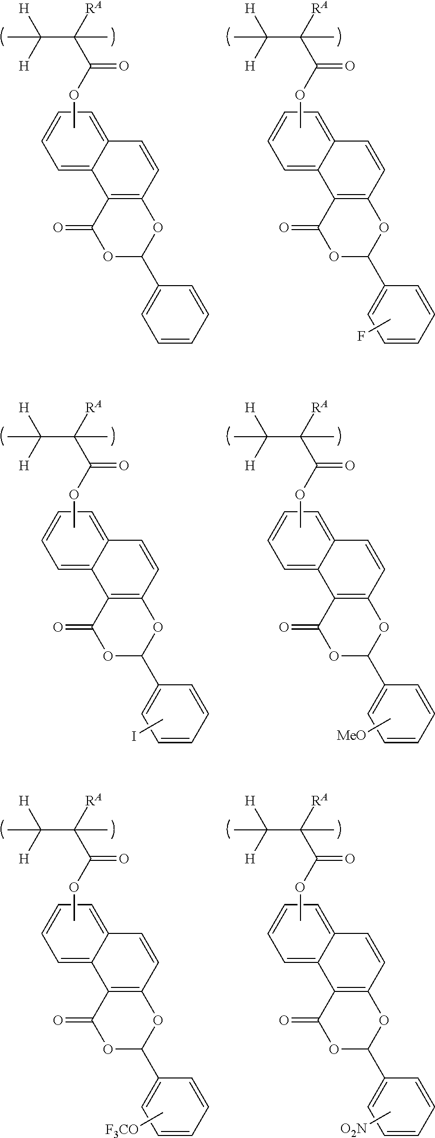

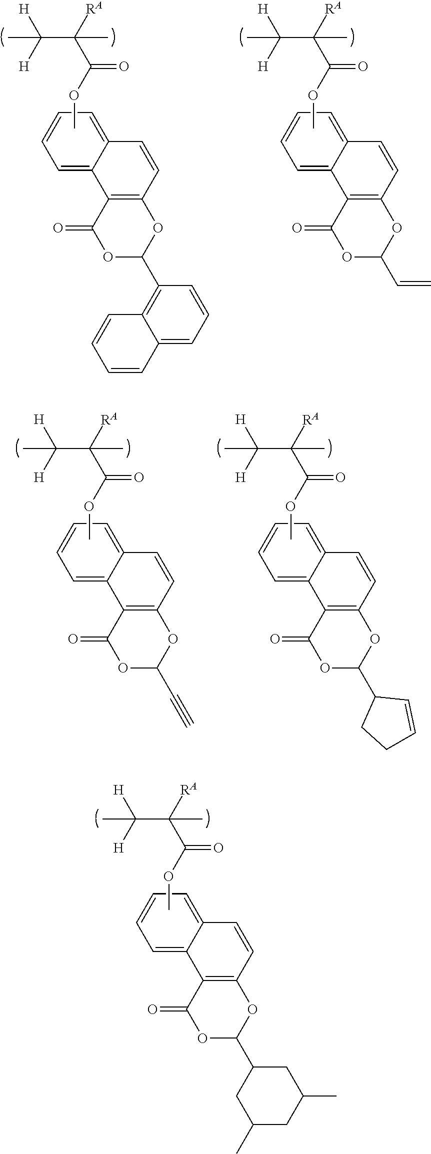

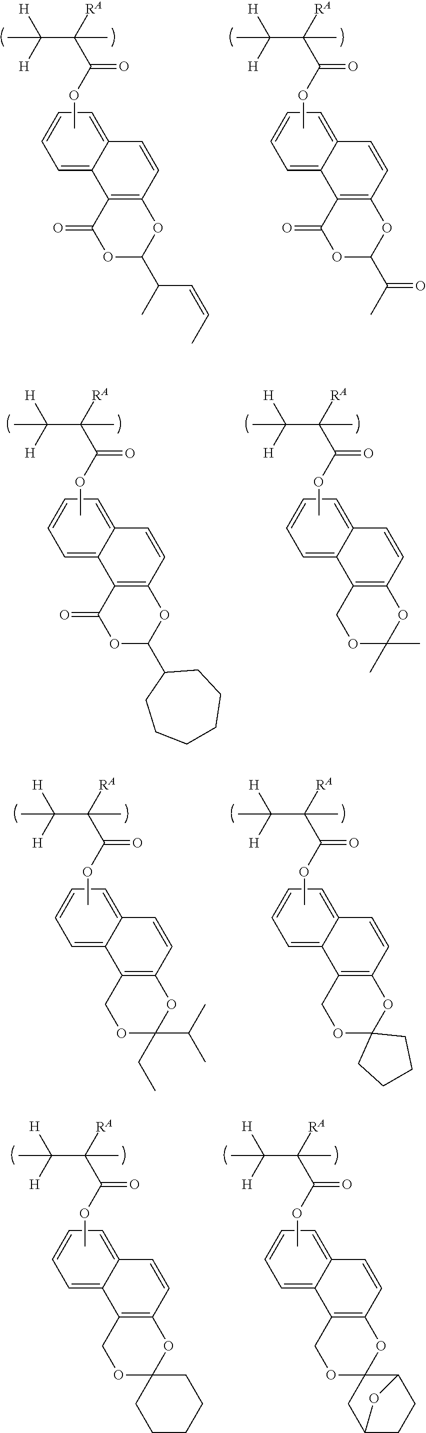

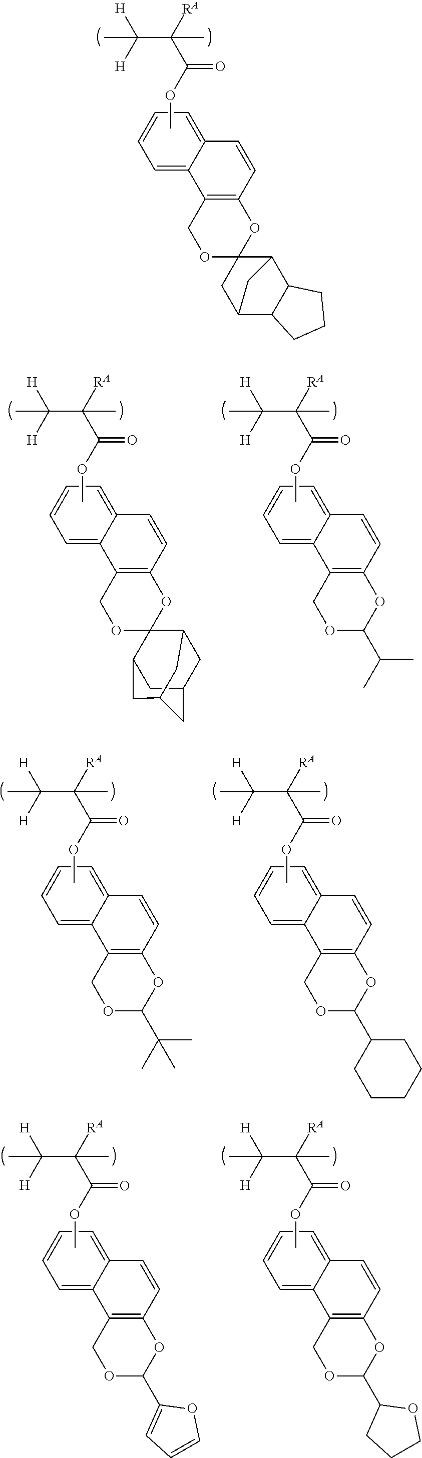

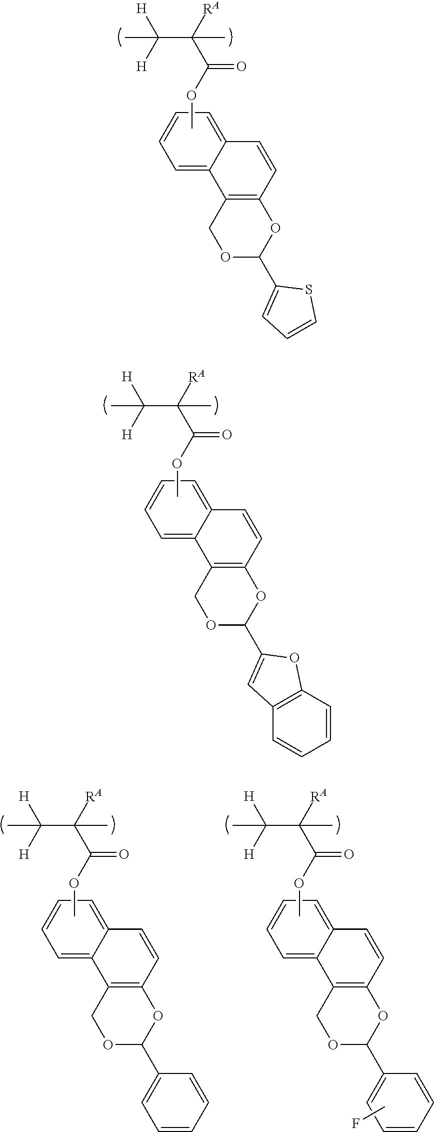

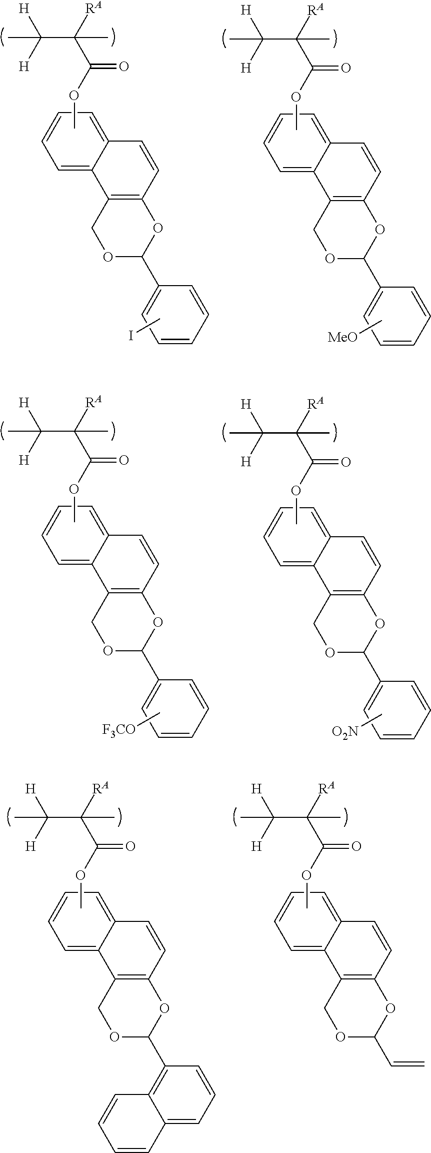

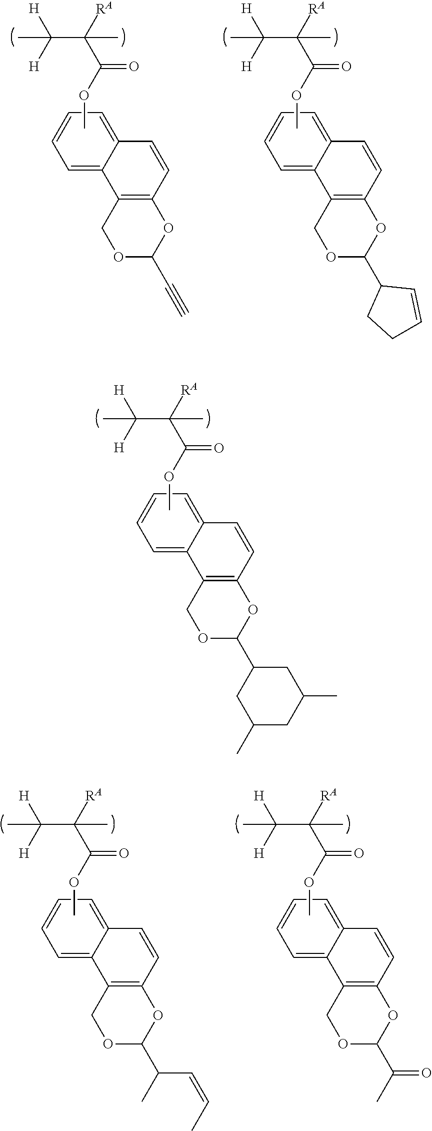

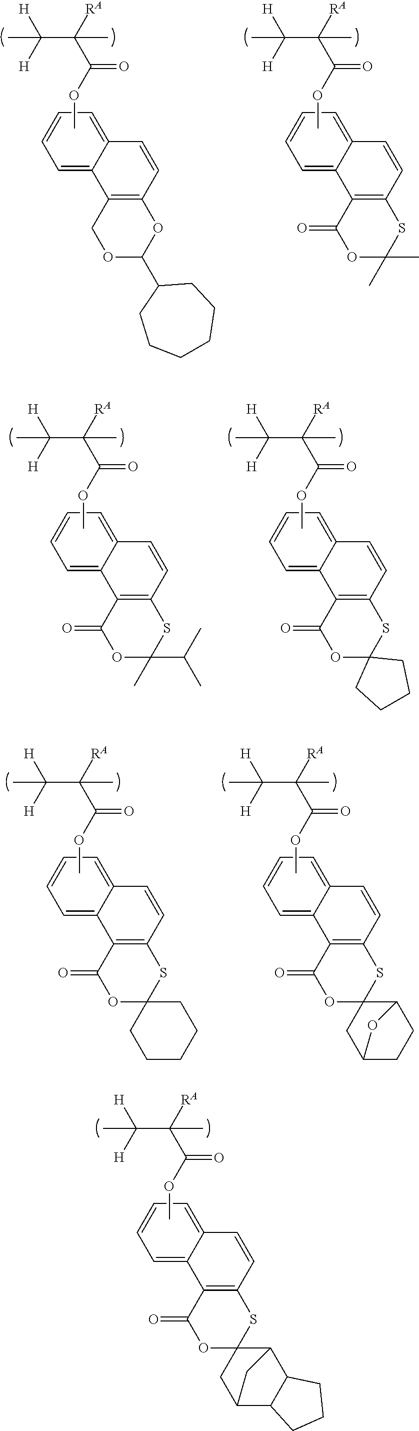

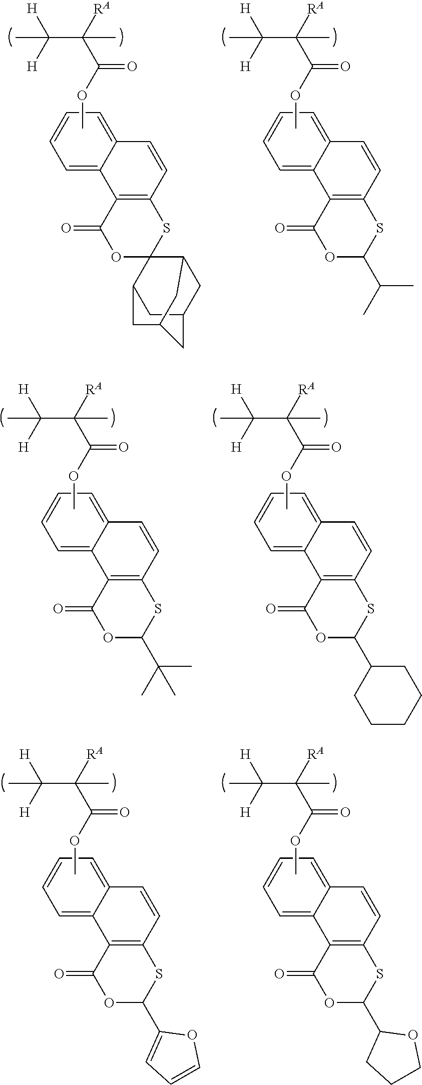

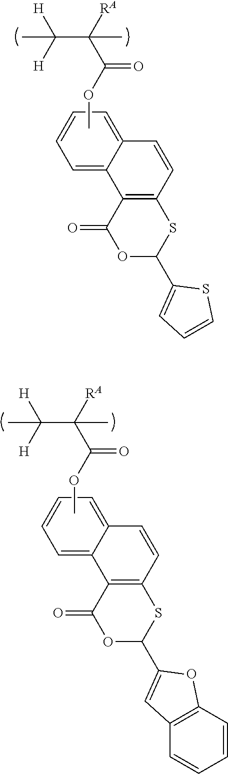

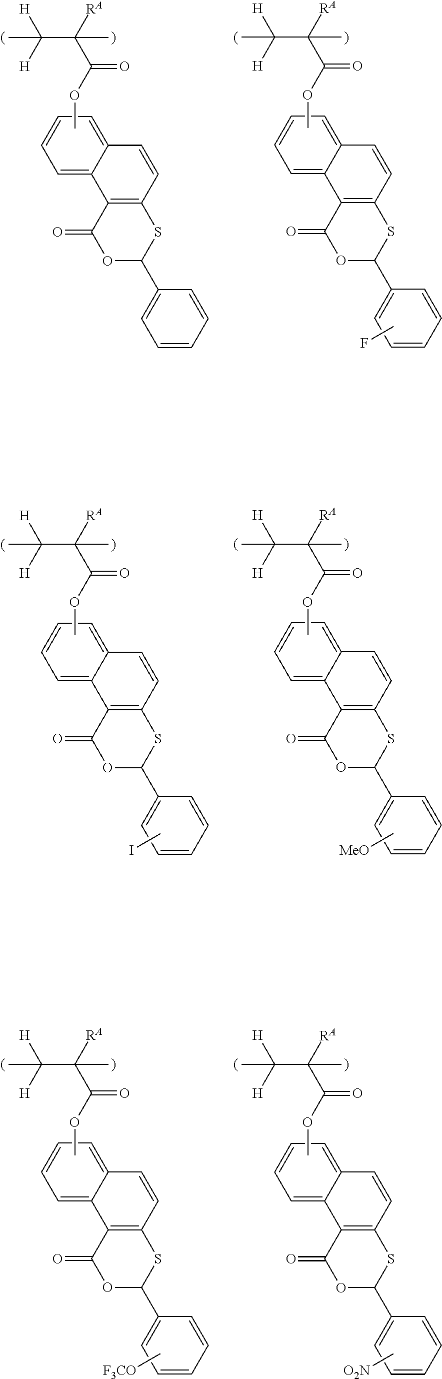

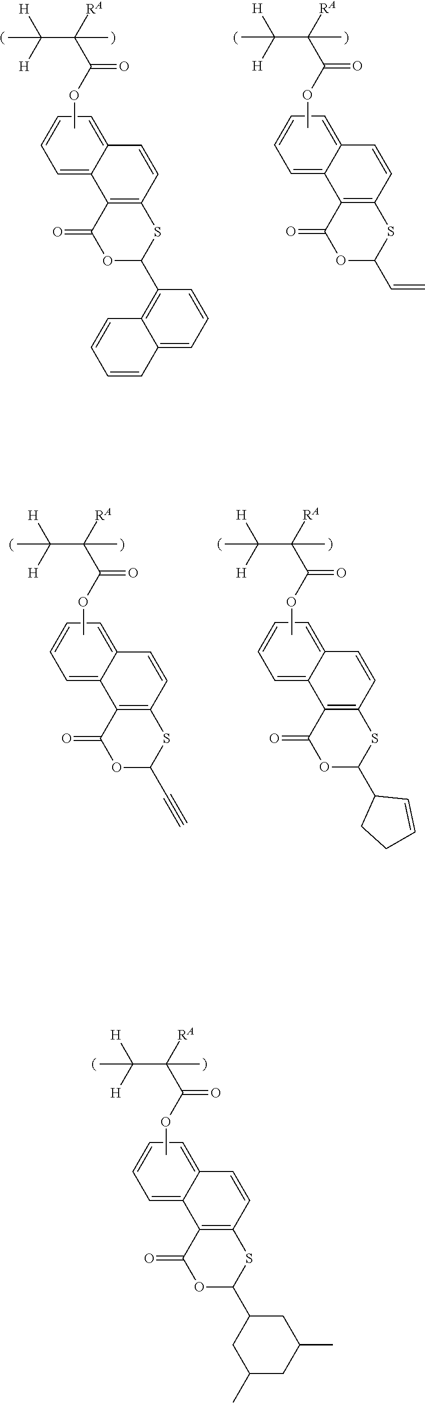

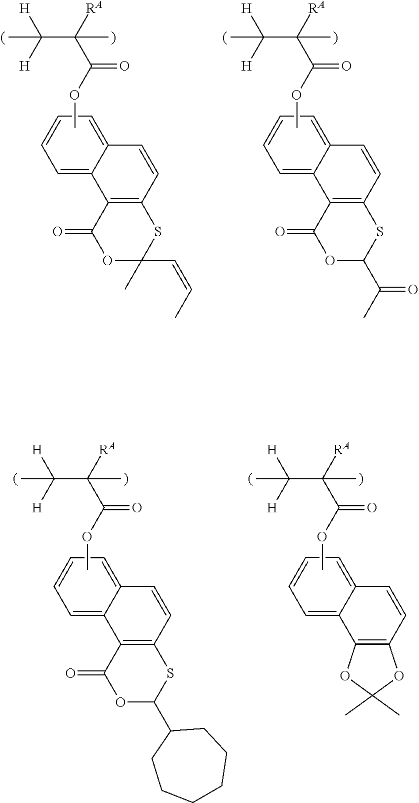

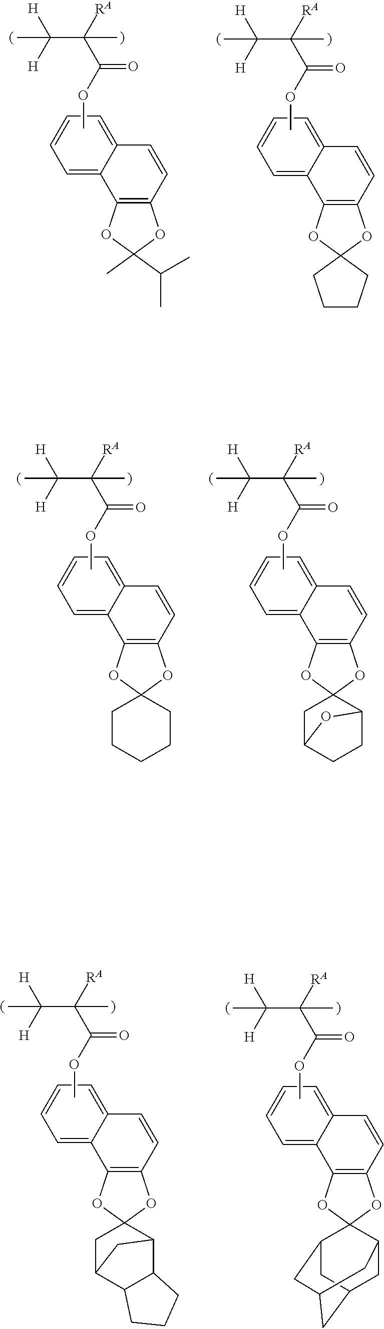

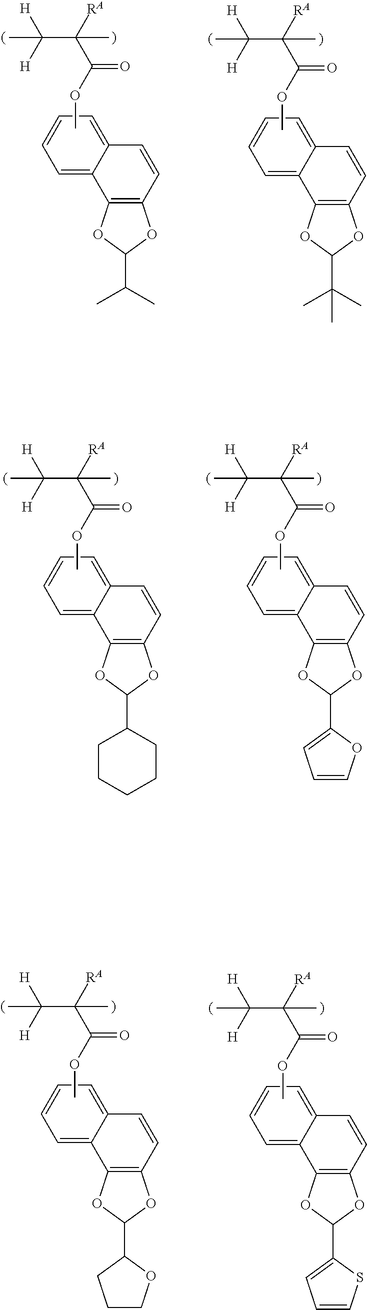

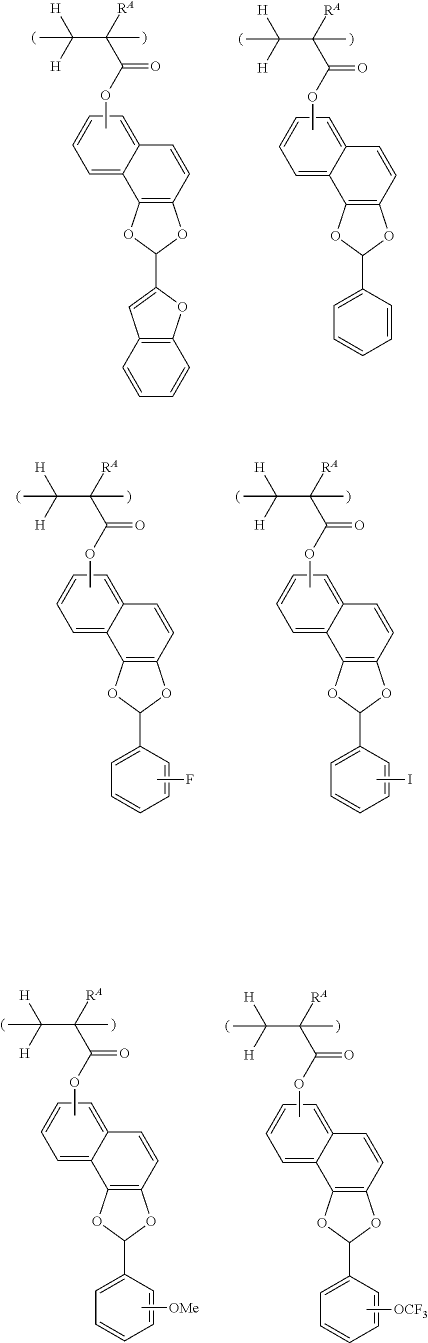

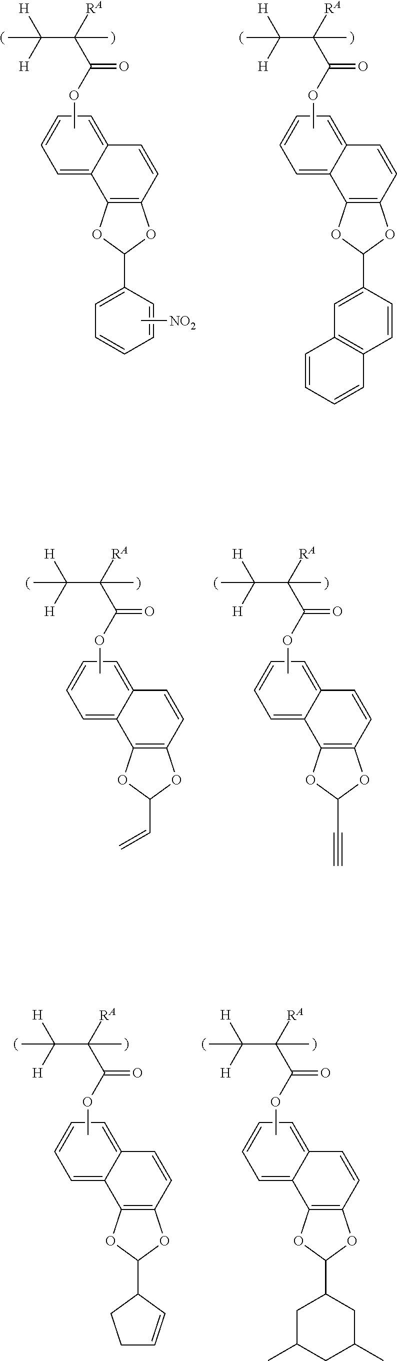

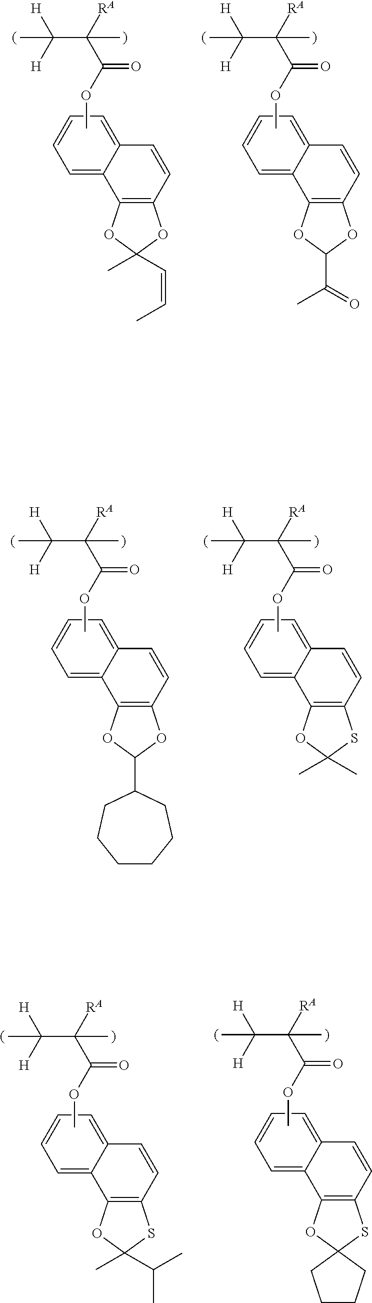

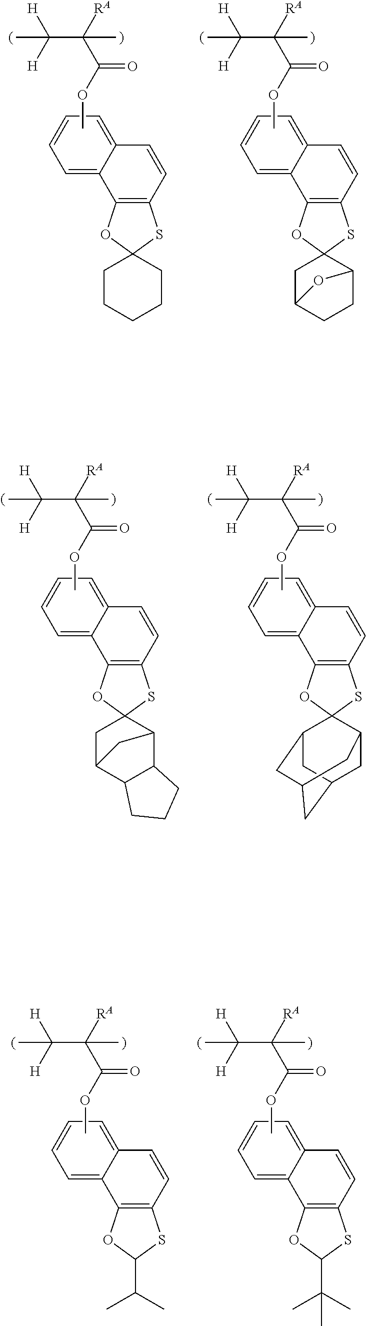

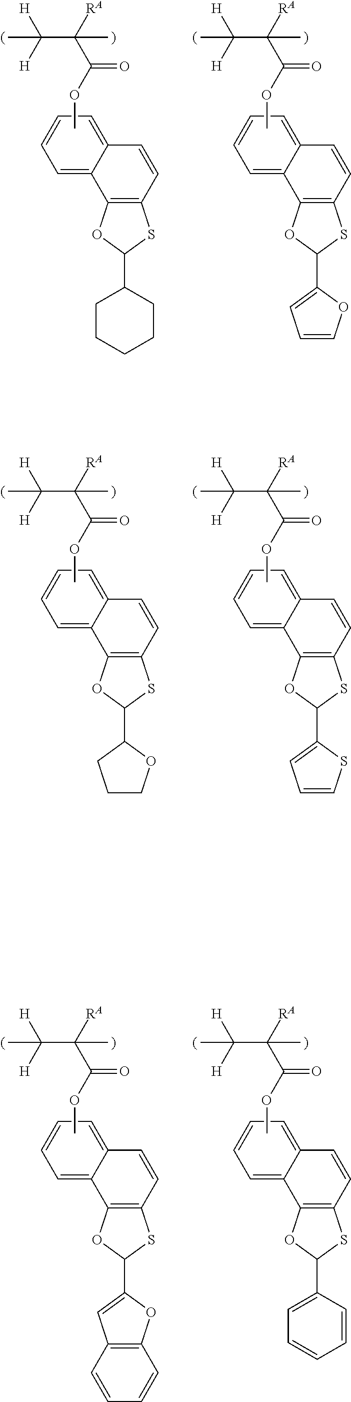

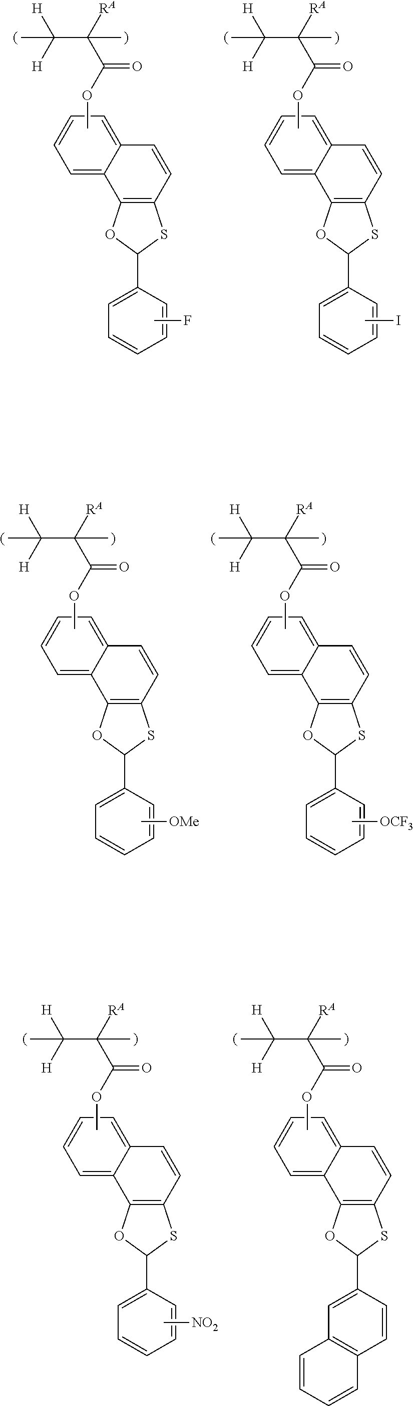

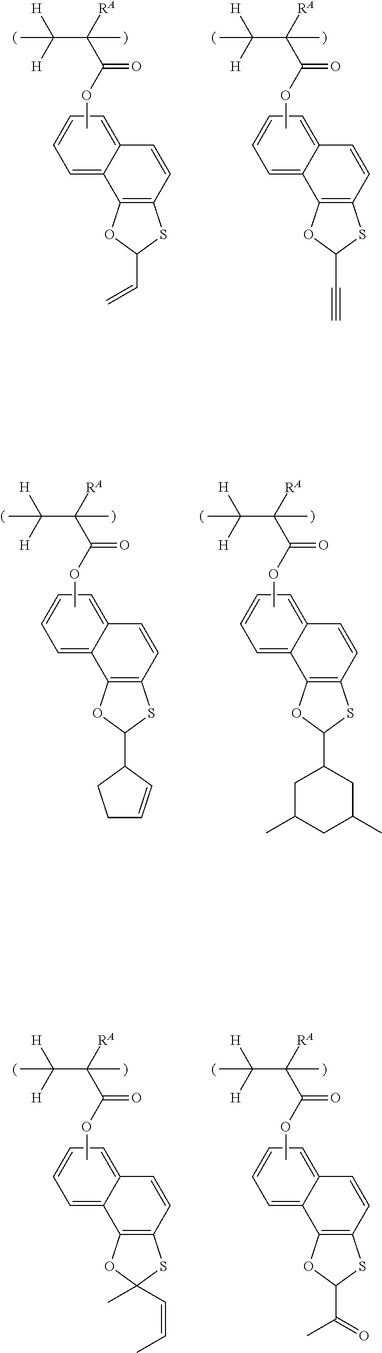

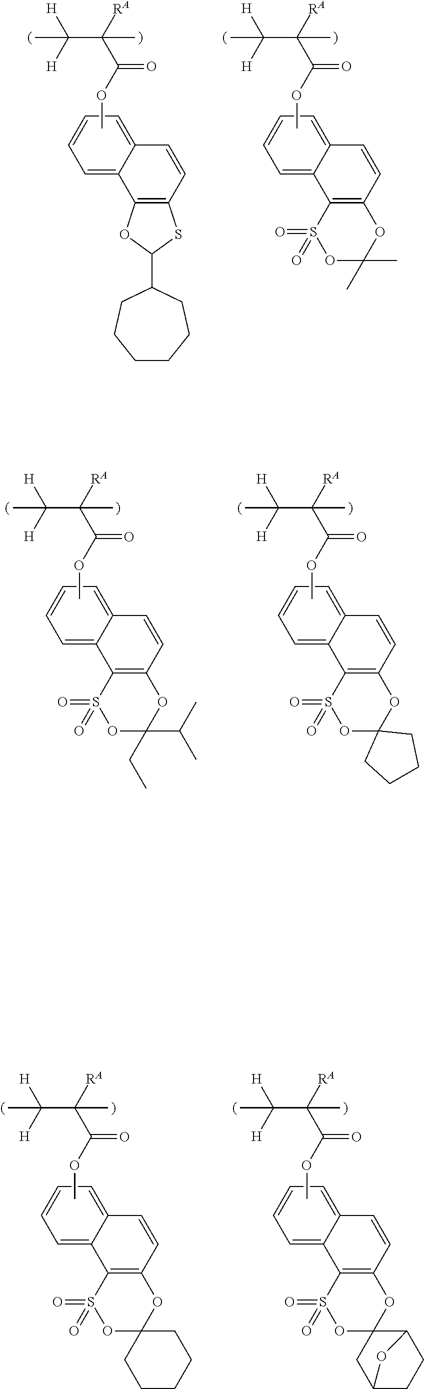

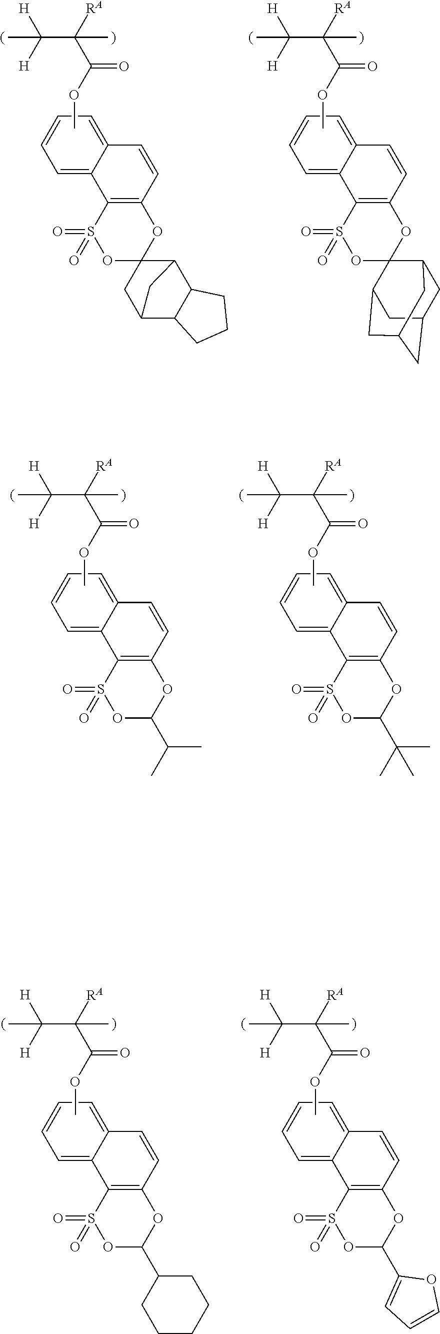

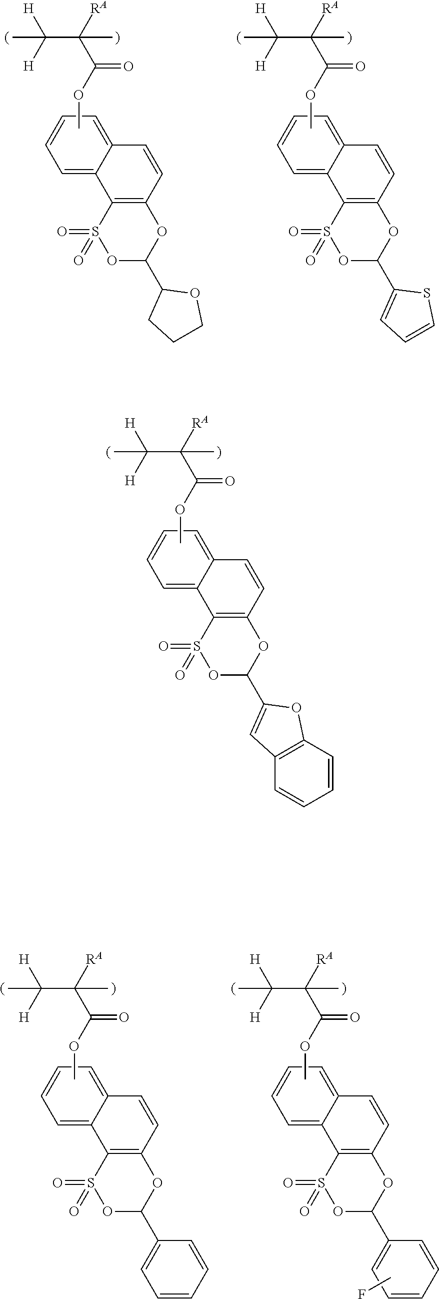

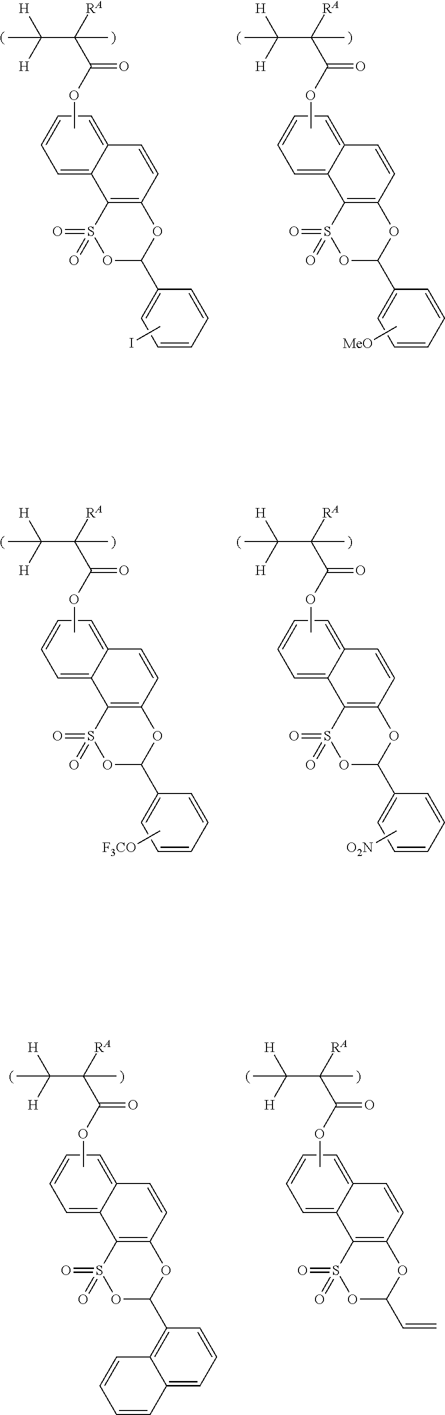

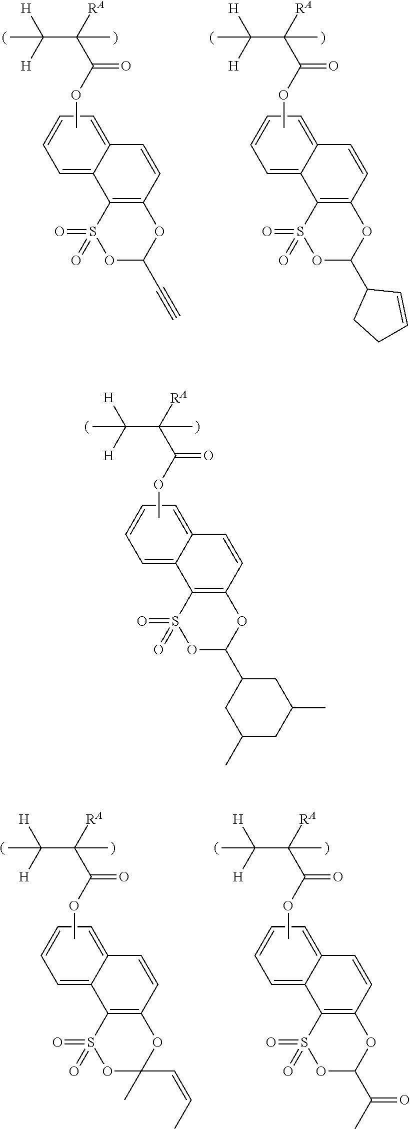

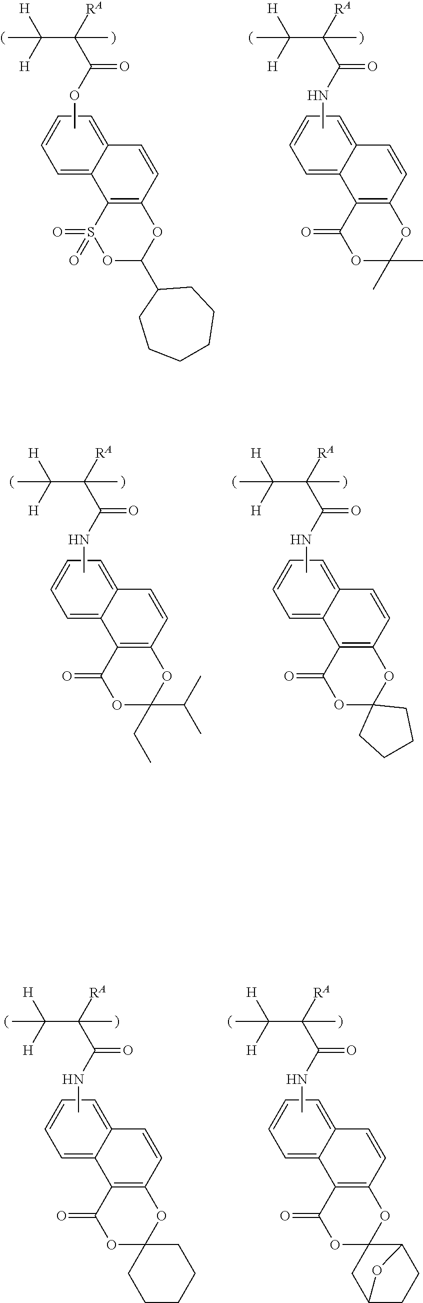

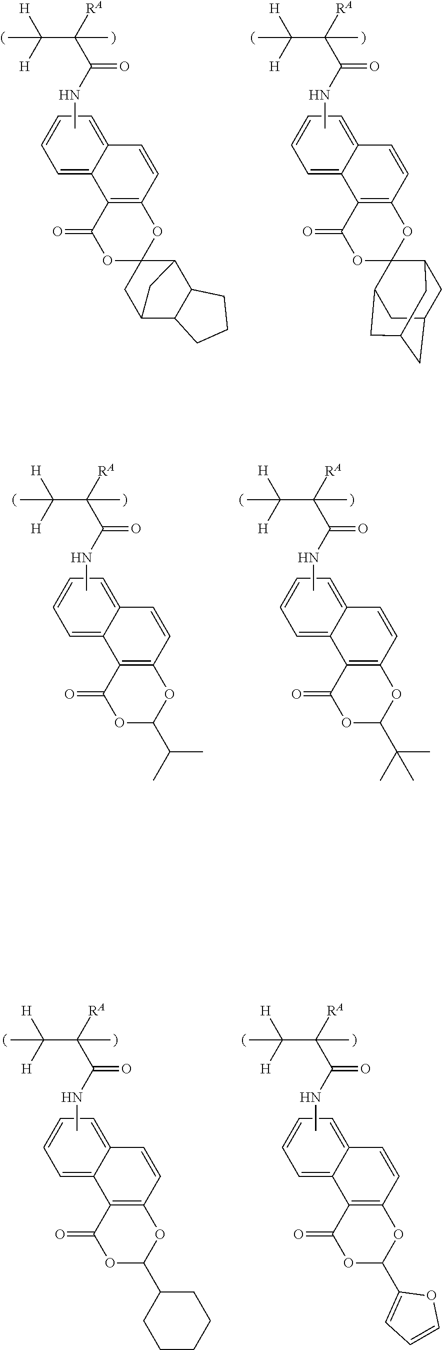

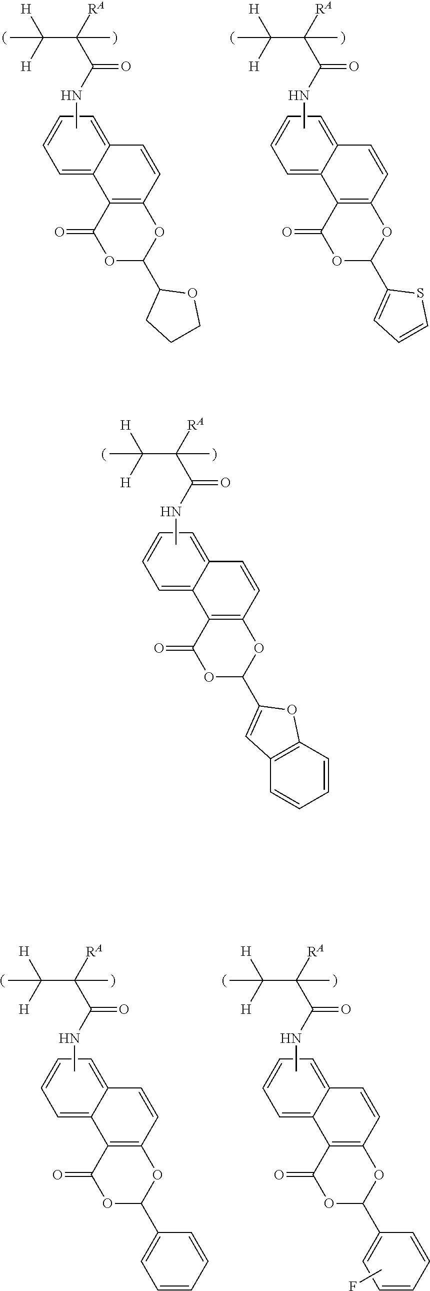

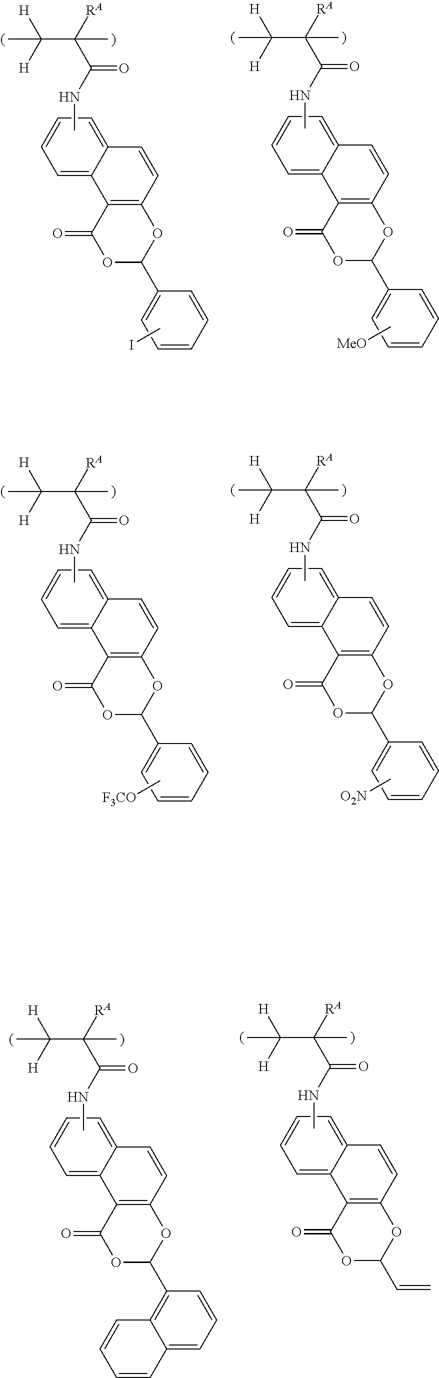

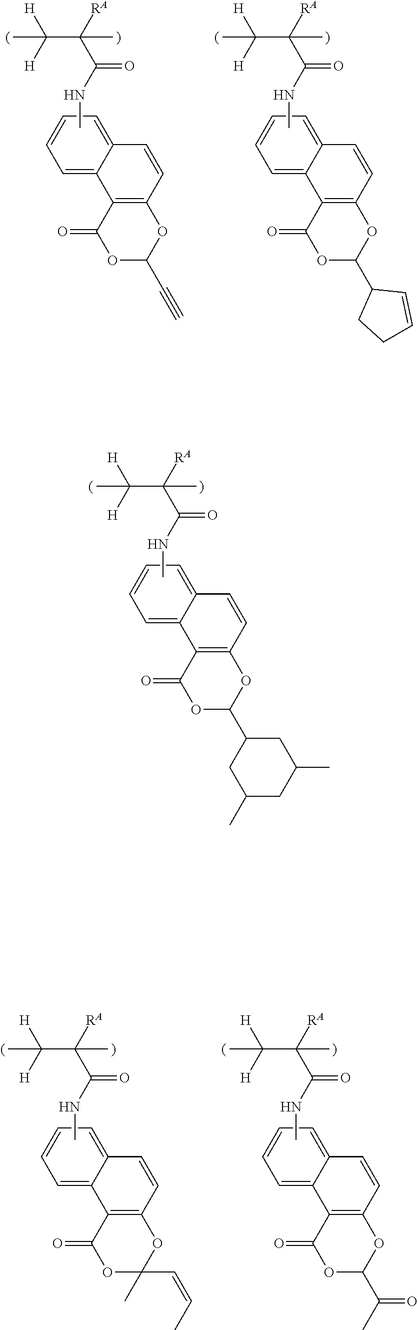

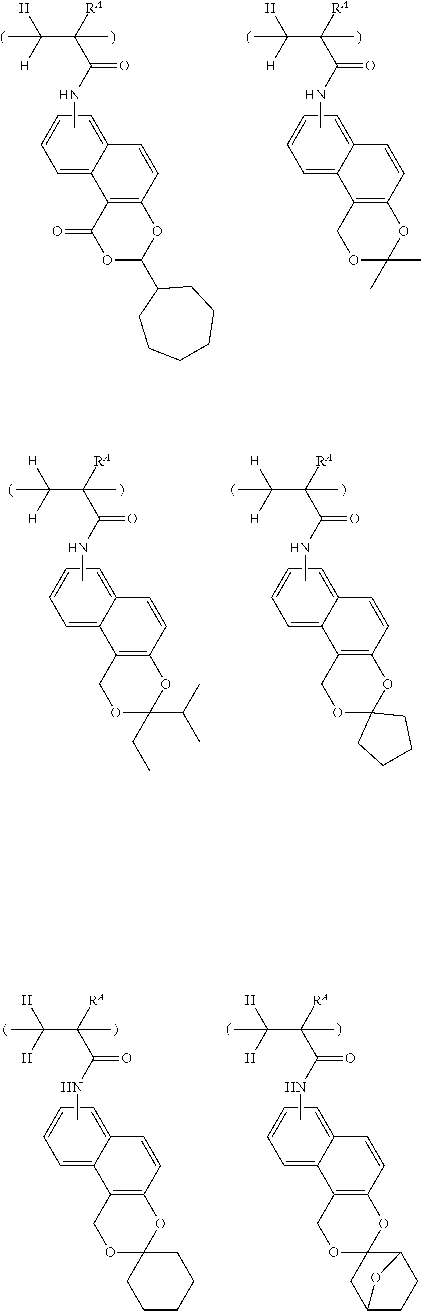

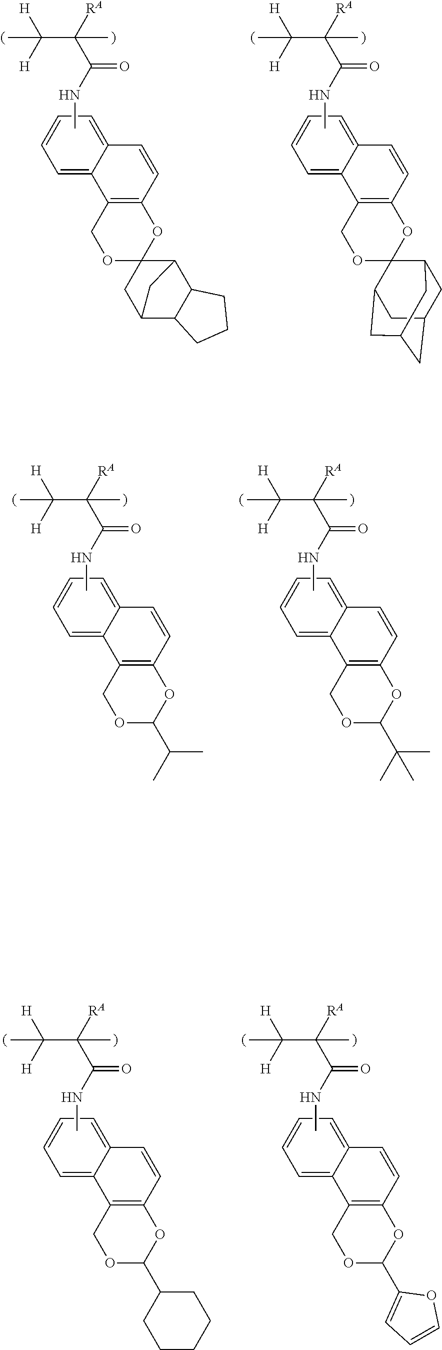

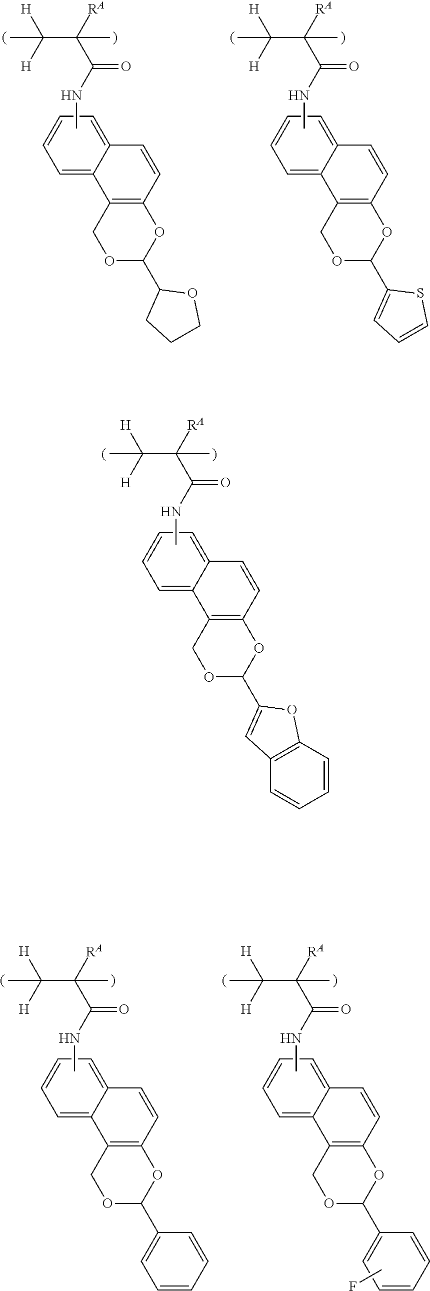

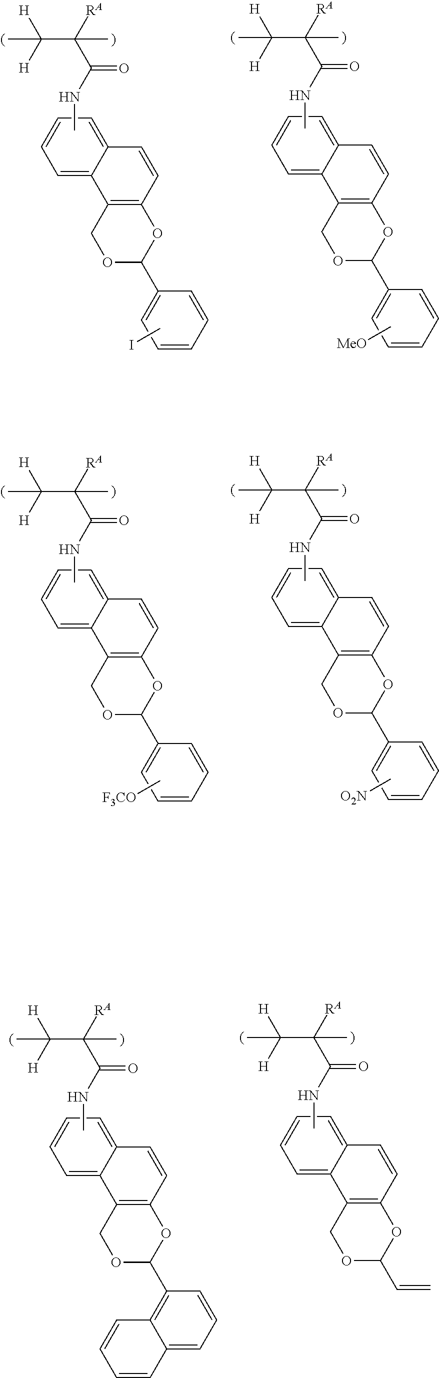

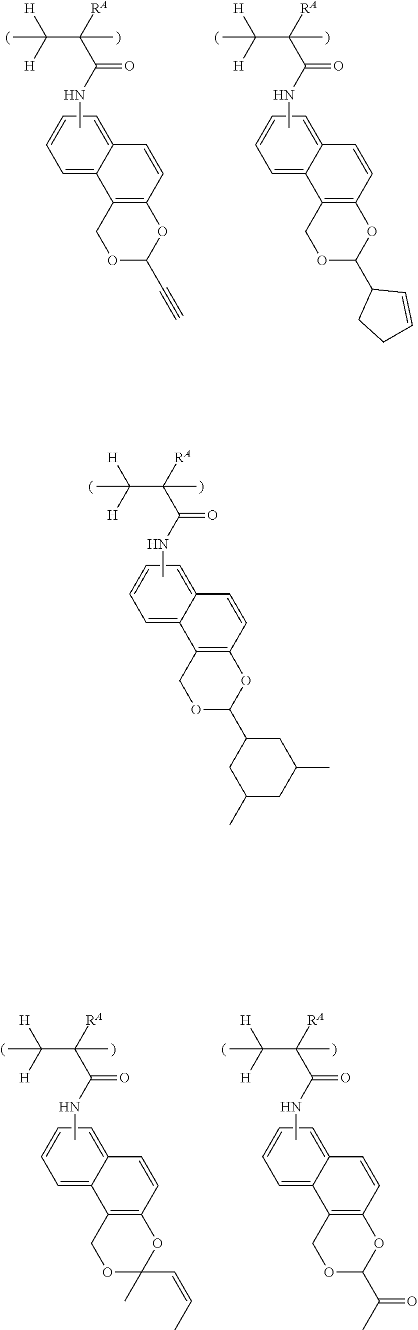

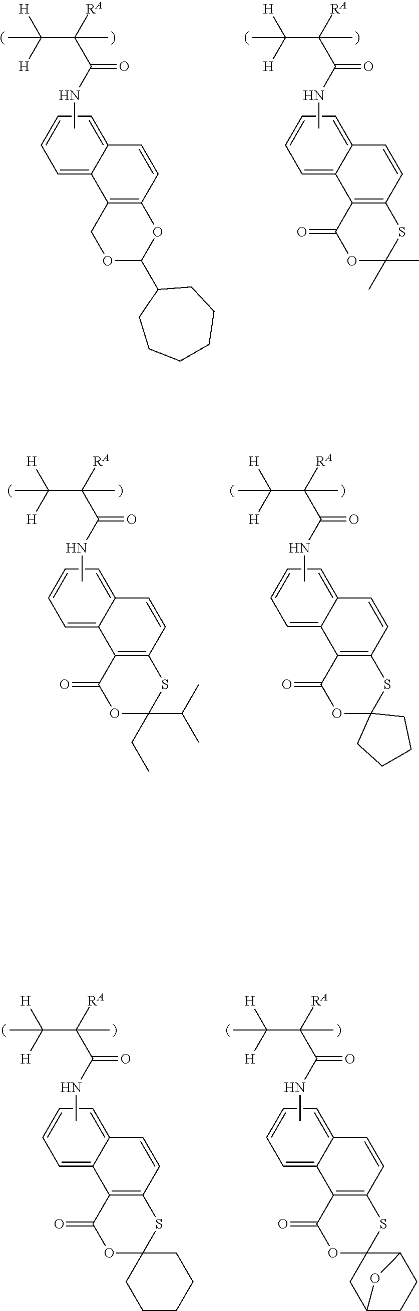

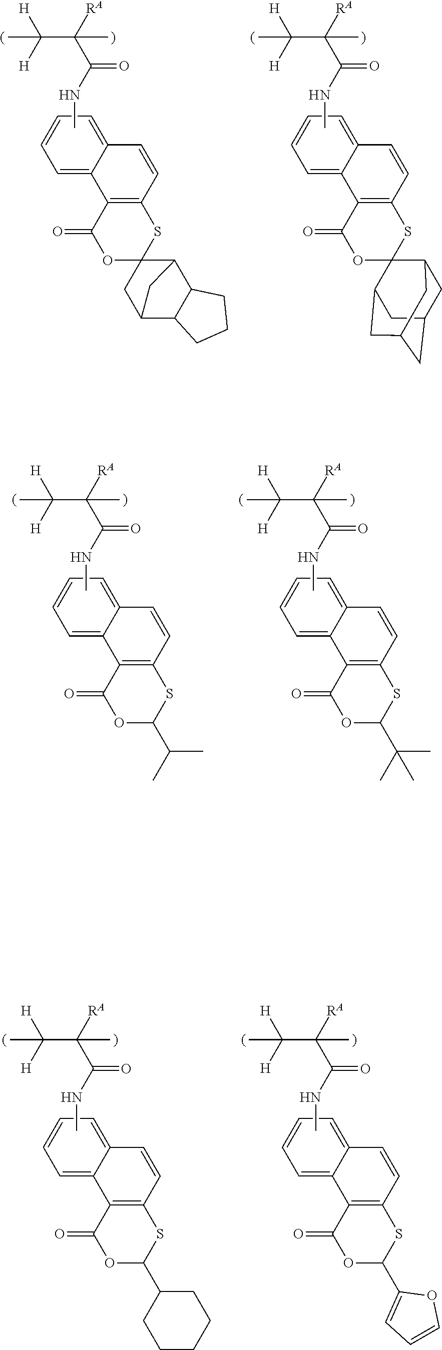

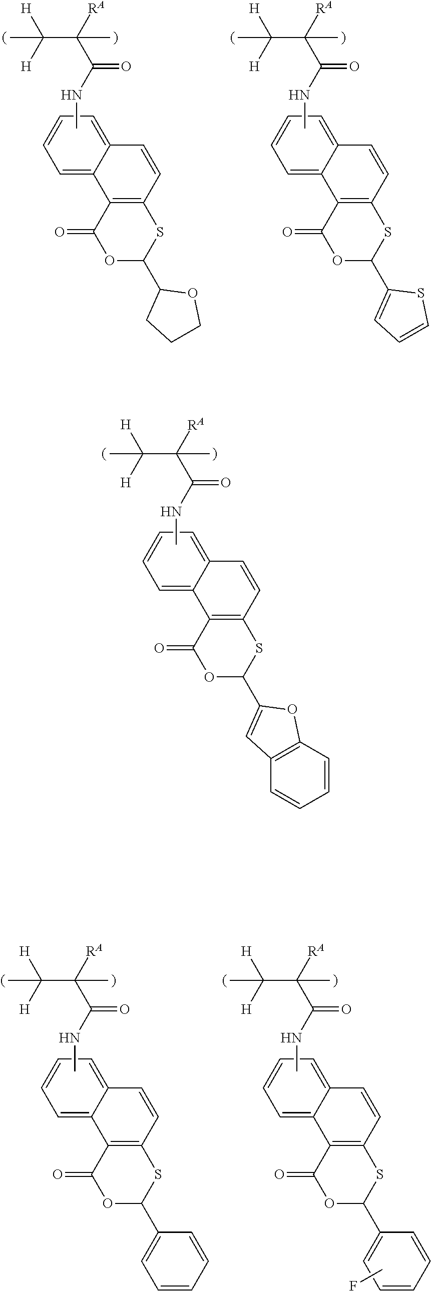

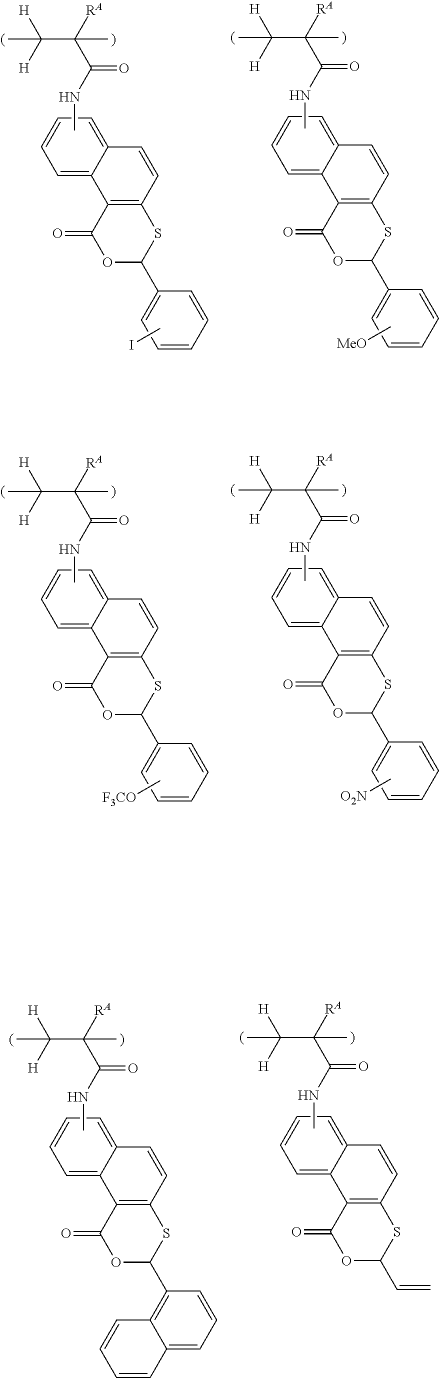

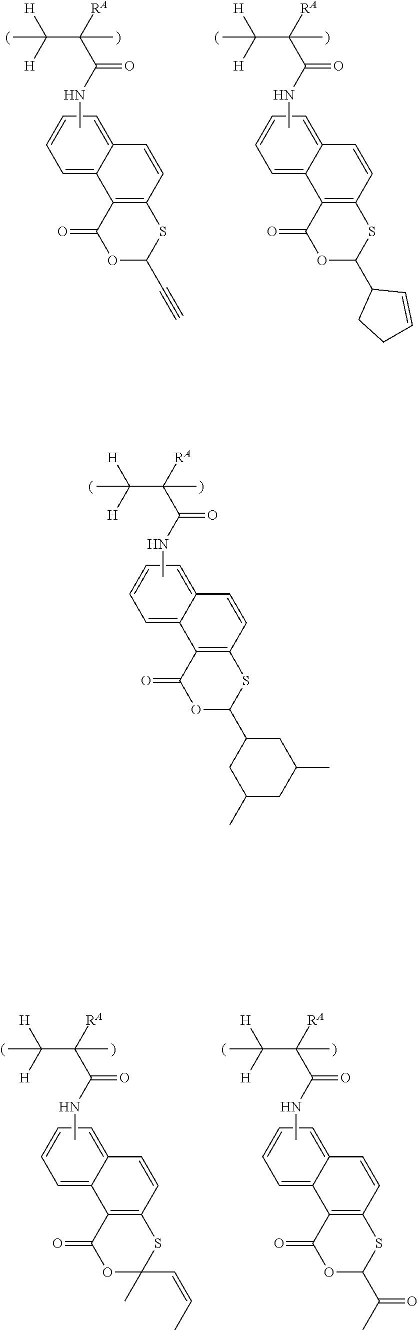

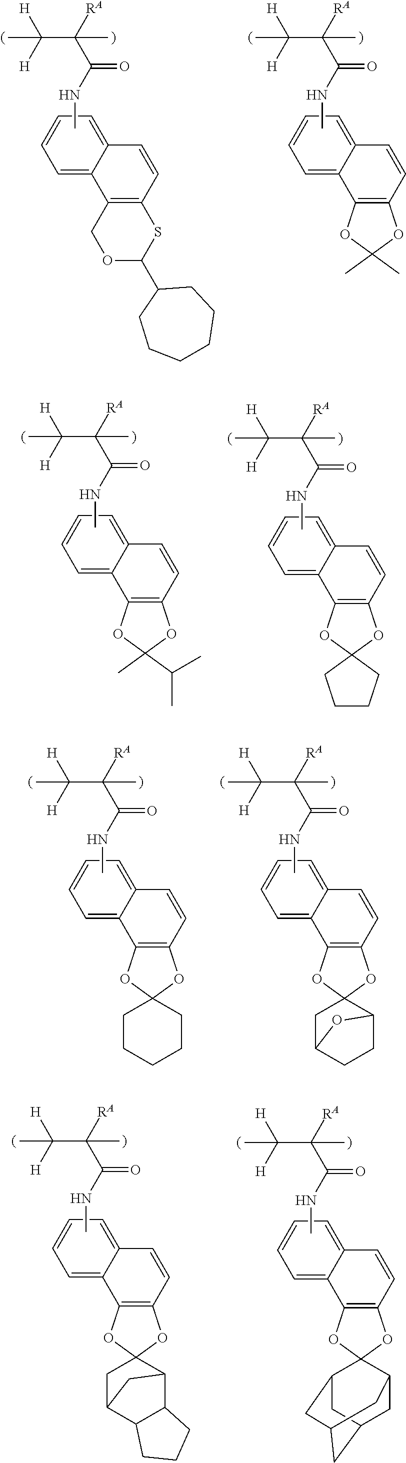

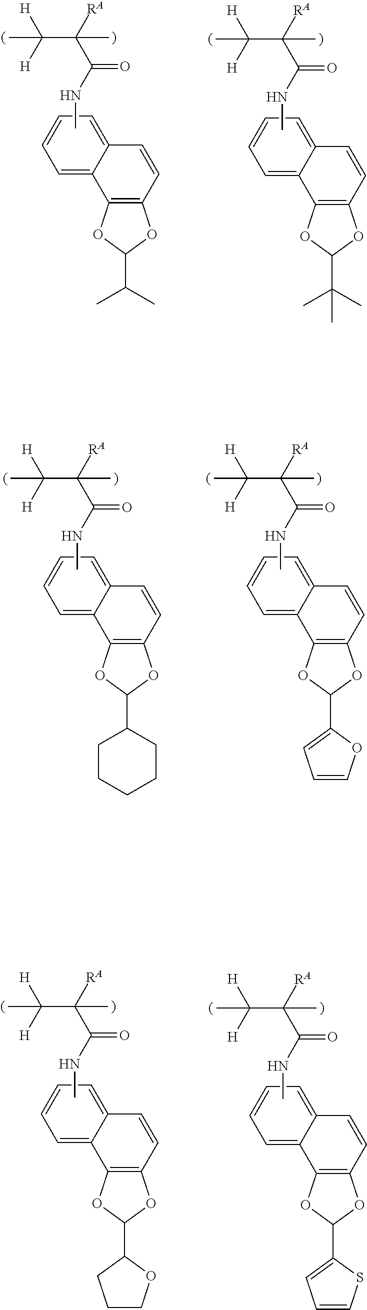

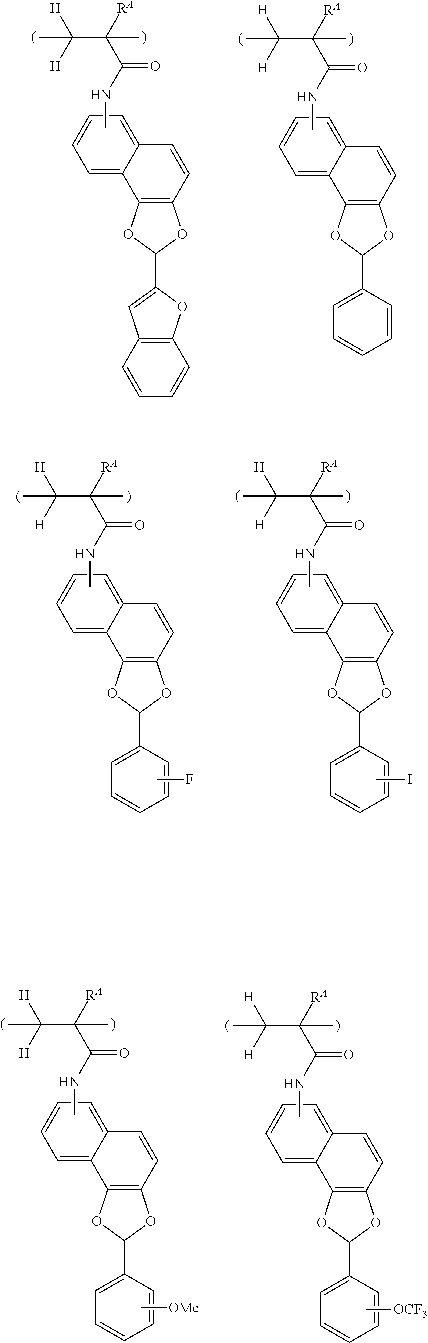

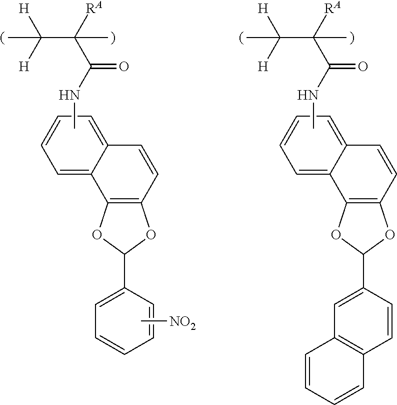

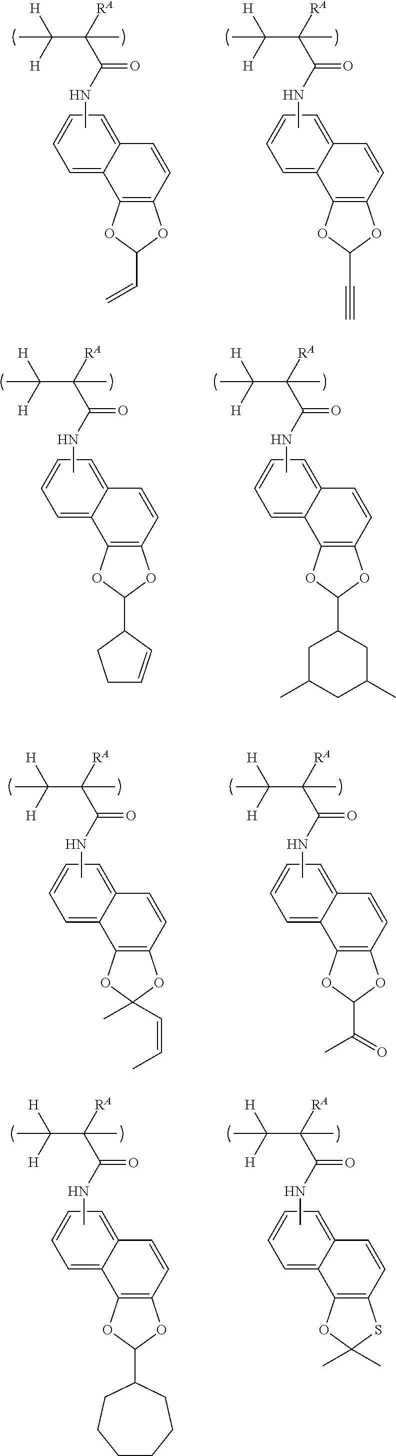

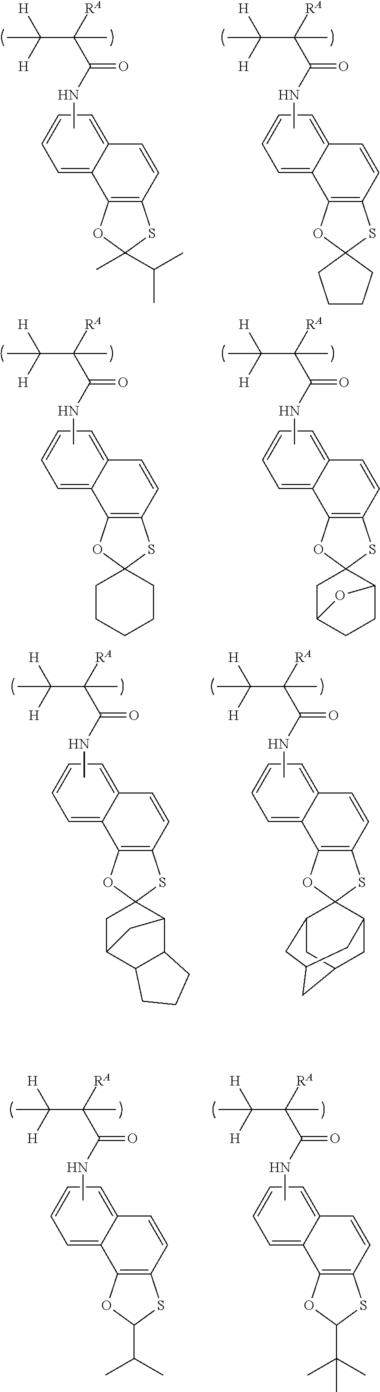

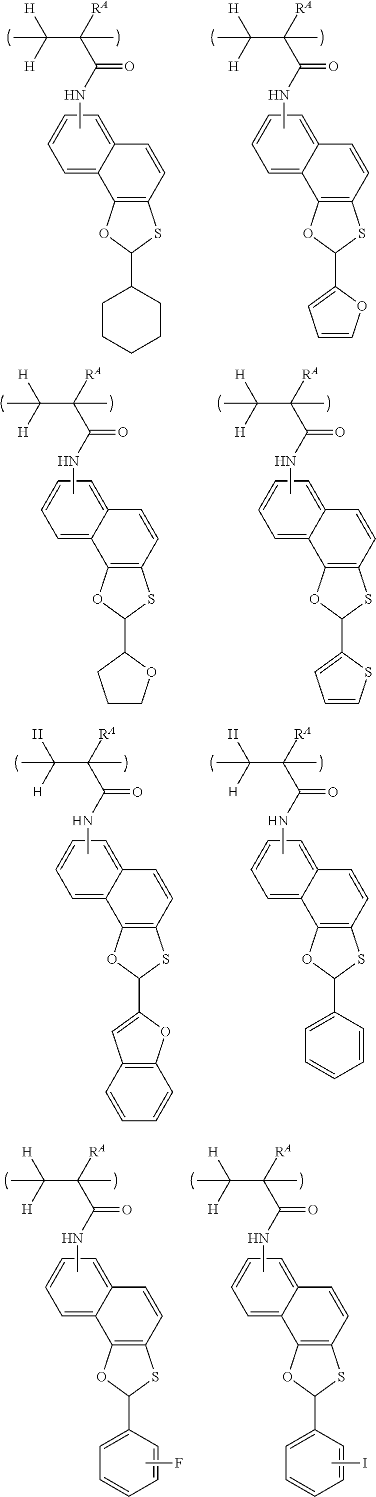

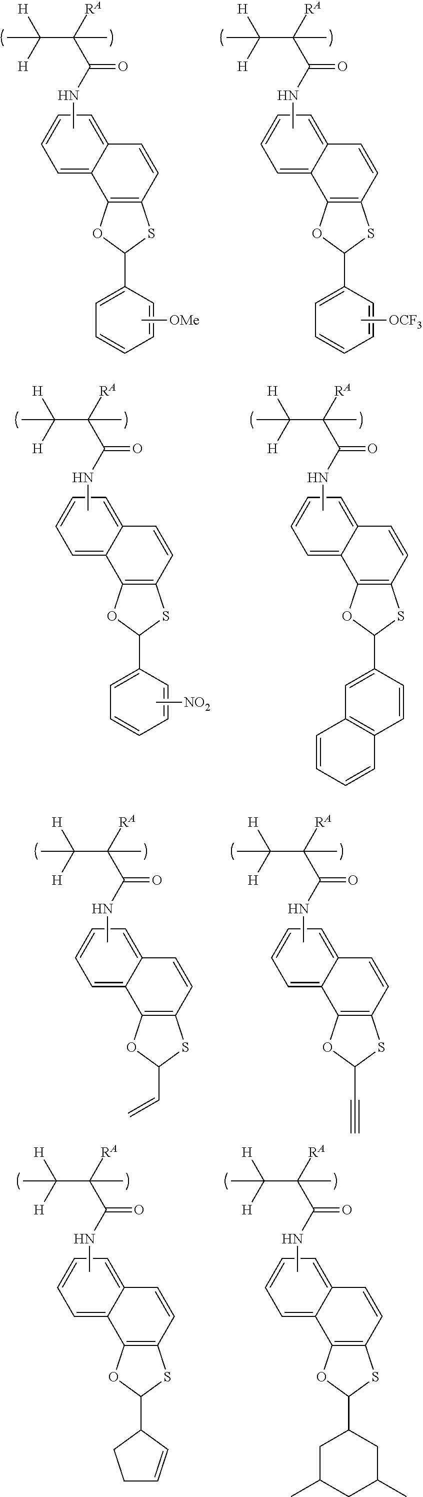

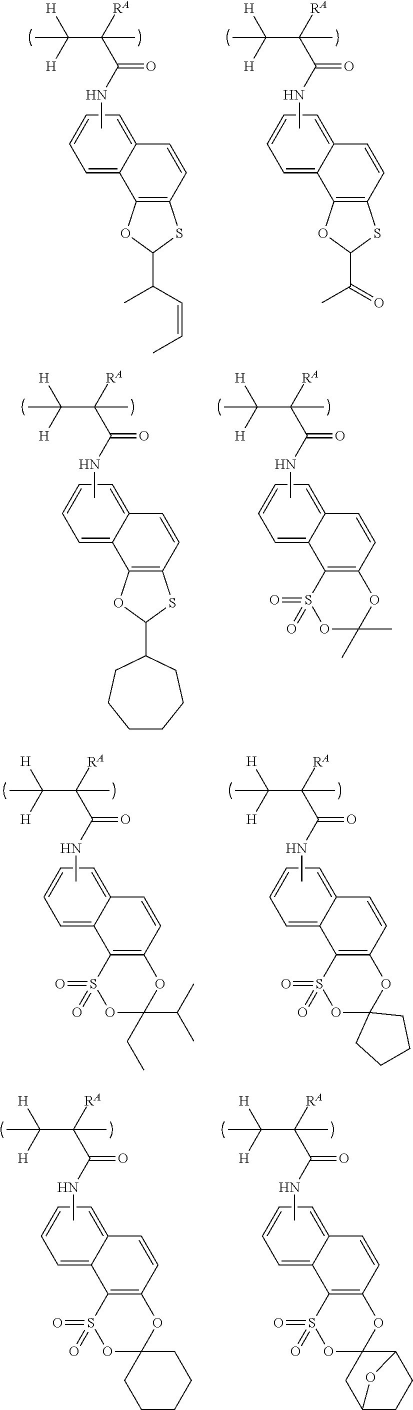

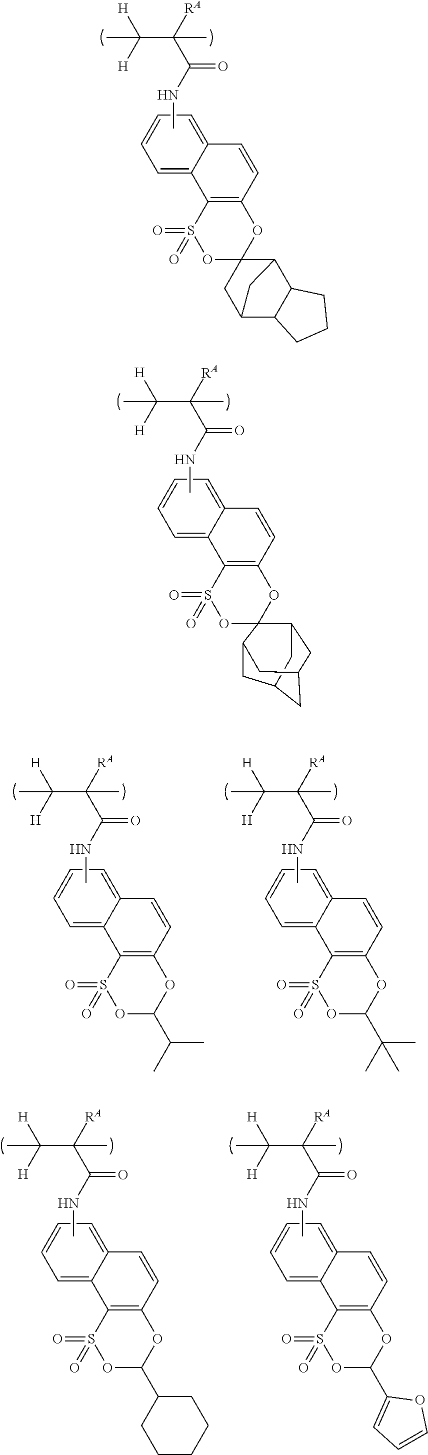

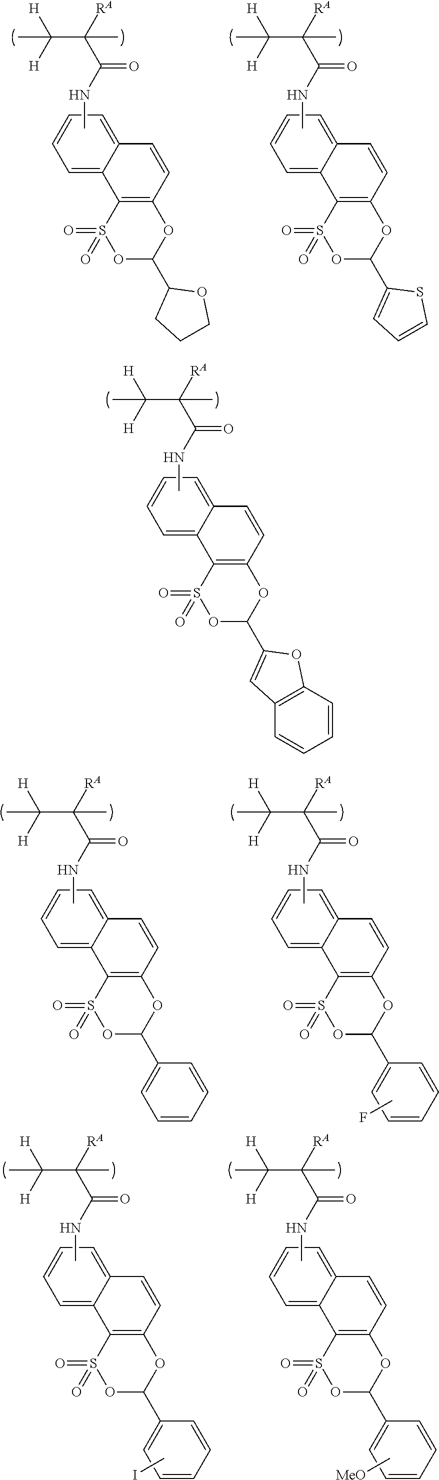

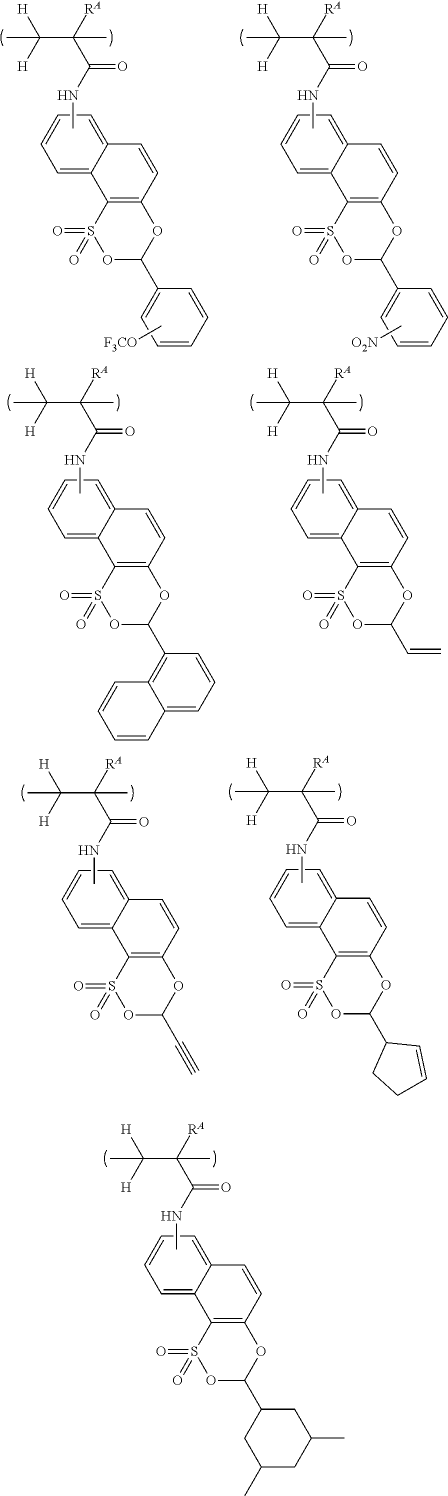

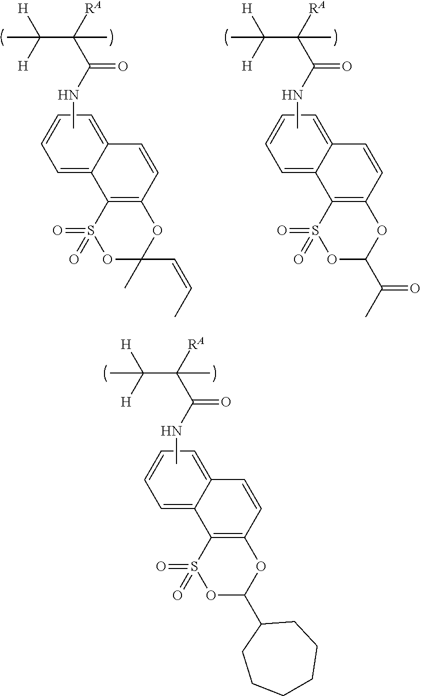

The polymer preferably further contains a repeating unit represented by the following general formula (c) (hereinafter, also referred to as repeating unit c).

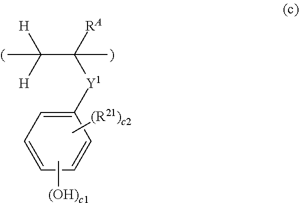

wherein R^{A} is a hydrogen atom, a fluorine atom, a methyl group, or a trifluoromethyl group;
Y¹ is a single bond, *—C(═O)—O—, or *—C(═O)—NH—, wherein * indicates bonding to a carbon atom of a main chain;
R²¹ is a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
c1 is an integer of 1 to 4, and c2 is an integer of 0 to 3, provided that 1≤c1+c2≤5 is satisfied.

In the general formula (c), R^{A} is a hydrogen atom, a fluorine atom, a methyl group, or a trifluoromethyl group. Y¹ is a single bond, *—C(═O)—O—, or *—C(═O)—NH—. * indicates bonding to a carbon atom of a main chain. R²¹ is a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. c1 is an integer of 1 to 4. c2 is an integer of 0 to 3. It should be noted that 1≤c1+c2≤5 is satisfied.

Specific examples of repeating unit c include, but are not limited to, those shown below. In the following formulae, R^{A} is as specified above.

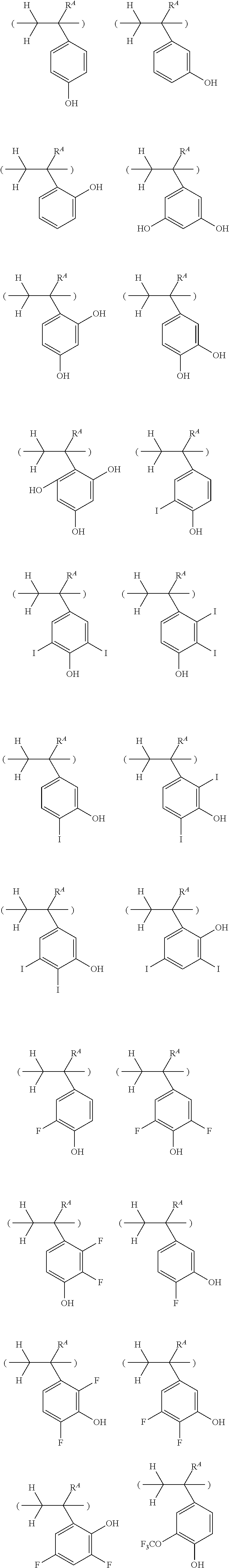

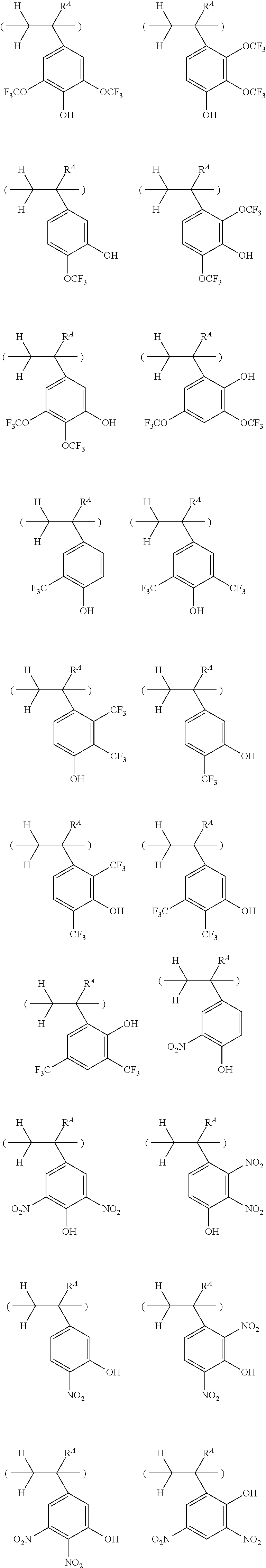

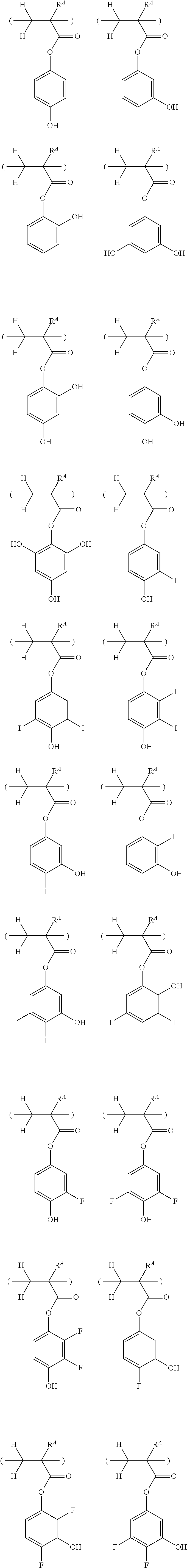

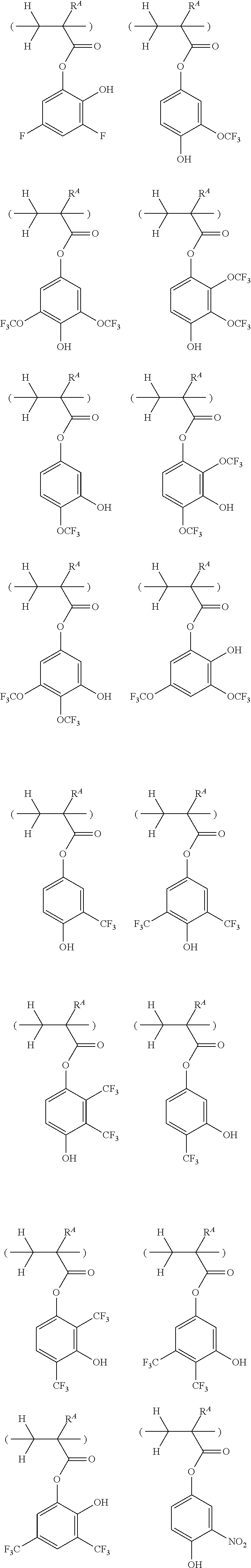

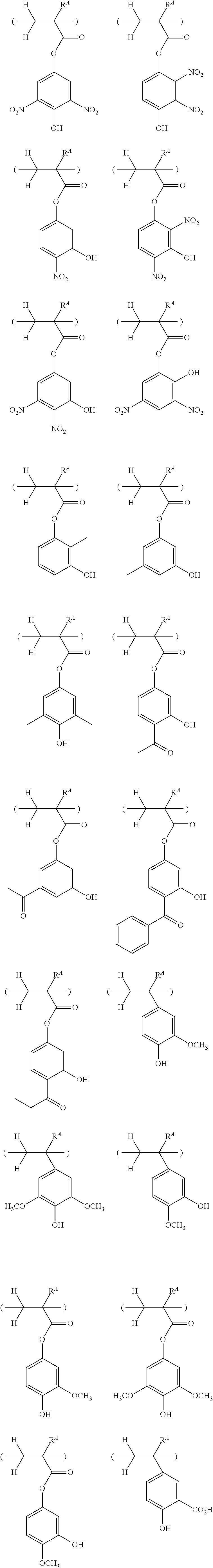

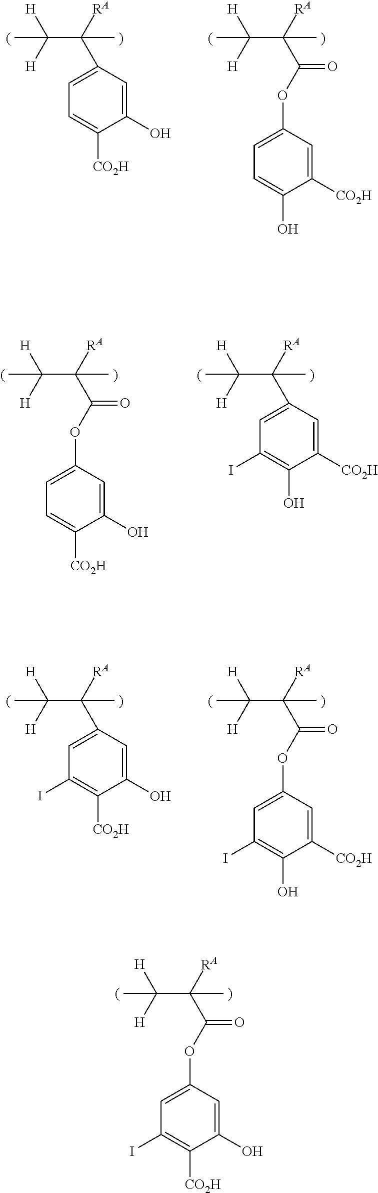

The polymer preferably further contains a repeating unit derived from an onium salt monomer containing a fluorosulfonate anion having a polymerizable group and at least one iodine atom and a sulfonium cation (hereinafter, also referred to as repeating unit d).

Specific examples of the anion of repeating unit d include structures shown in paragraphs [0023] to [0029] of JP 6973274 B, paragraphs [0032] to [0038] of JP 2023-172918 A, and paragraphs [0032] to [0047] of JP 2024-043941 A.

In addition, as a specific example of the anion of repeating unit d, one represented by the following formula (d1) is preferred.

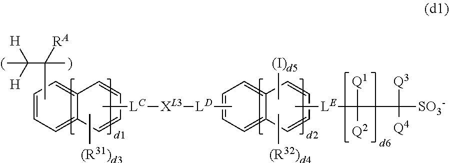

In the general formula (d1), d1 is 0 or 1. While a benzene ring is given if d1 is 0, and a naphthalene ring is given if d1 is 1, the case that d1 is 0 to give a benzene ring is preferred for solubility in solvents. d2 is 0 or 1. While a benzene ring is given if d2 is 0, and a naphthalene ring is given if d2 is 1, the case that d2 is 0 to give a benzene ring is preferred for solubility in solvents. d3 is 0, 1, 2, 3, or 4. d3 is preferably 0, 1, or 2, and further preferably 0 or 1 for raw material procurement. d4 is 0, 1, 2, 3, or 4, preferably 0, 1, 2, or 3, more preferably 0, 1, or 2, and further preferably 0 or 1. d5 is 1, 2, 3, 4, 5, or 6. More iodine atoms in the anion structure result in enhanced absorption particularly to EUV, but in poor solubility in solvents, which may disadvantageously cause precipitation in the resist composition, and for this reason d5 is preferably 1, 2, or 3, and more preferably 1 or 2. It should be noted that 1≤d4+d5≤4 is satisfied if d2 is 0, and 1≤d4+d5≤6 is satisfied if d2 is 1. d6 is 0, 1, 2, 3, or 4, preferably 0, 1, 2, or 3, and more preferably 1.

In the general formula (d1), R^{A} is a hydrogen atom, a fluorine atom, a methyl group, or a trifluoromethyl group. Among them, R^{A} is preferably a hydrogen atom or a methyl group, and more preferably a hydrogen atom.

In the general formula (d1), each iodine atom in an aromatic ring of the anion is preferably bound to the ortho position of the carbon atom to which L^{D} is bound. Iodine is an element with a large atomic radius, and hence inhibits the rotation of the bond axis between the aromatic ring to which a polymerizable group is bound and the aromatic ring to which an iodine atom or iodine atoms are bound, imparting rigidity to the polymer.

In the general formula (d1), R³¹ is a halogen atom or a hydrocarbyl group having 1 to 20 carbon atoms and optionally containing a heteroatom. The halogen atom is preferably a fluorine atom, a chlorine atom, a bromine atom, or an iodine atom, and more preferably a fluorine atom or an iodine atom. The hydrocarbyl group may be saturated or unsaturated, and may be linear, branched, or cyclic. Specific examples thereof include an alkyl group having 1 to 20 carbon atoms such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; a cyclic saturated hydrocarbyl group having 3 to 20 carbon atoms such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; an alkenyl group having 2 to 20 carbon atoms such as a vinyl group, an allyl group, a propenyl group, a butenyl group, and a hexenyl group; a cyclic unsaturated hydrocarbyl group having 3 to 20 carbon atoms such as a cyclohexenyl group; an aryl group having 2 to 20 carbon atoms such as a phenyl group and a naphthyl group; an aralkyl group having 7 to 20 carbon atoms such as a benzyl group, a 1-phenylethyl group, and a 2-phenylethyl group; and a group given by combining any of these. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained. If d3 is 2, 3, or 4, R³¹ groups are identical to or different from each other.

If d3 is 2, 3, or 4, any multiple R³¹ groups are optionally bound together to form a ring together with the carbon atoms of the aromatic ring or aromatic rings to which the groups are bound. Specific examples of the ring formed in this case include a cyclopropane ring, a cyclobutane ring, a cyclopentane ring, a cyclohexane ring, a norbornane ring, and an adamantane ring. Some or all of the hydrogen atoms in the ring may be each replaced with a group containing a heteroatom such as an oxygen atom, a sulfur atom, a nitrogen atom, and a halogen atom, some of the —CH₂— moieties in the ring may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

In the general formula (d1), R³² is a halogen atom being not an iodine atom or a hydrocarbyl group having 1 to 20 carbon atoms and optionally containing a heteroatom. Specific examples of the halogen atom being not an iodine atom include a fluorine atom, a chlorine atom, and a bromine atom. The hydrocarbyl group may be saturated or unsaturated, and may be linear, branched, or cyclic. Specific examples thereof include, but are not limited to, those shown as examples of the hydrocarbyl group represented by R³¹. If d4 is 2, 3, or 4, R³² groups are identical to or different from each other.

If d4 is 2, 3, or 4, any multiple R³² groups are optionally bound together to form a ring together with the carbon atoms to which the groups are bound. The ring is preferably a 5- to 8-membered ring.

In the general formula (d1), L^{C}, L^{D}, and L^{E} are each independently a single bond, an ether bond, an ester bond, a sulfonate bond, a sulfonamide bond, a carbonate bond, or a carbamate bond. Among them, L^{C} is preferably a single bond, an ether bond, an ester bond, or a sulfonate bond, and more preferably an ester bond or a sulfonate bond. L^{D} is preferably a single bond, an ether bond, or an ester bond, and more preferably a single bond. L^{E} is preferably a single bond, an ether bond, an ester bond, or a sulfonate bond, and more preferably an ether bond or an ester bond.

In the general formula (d1), X^{L3} is a single bond or a hydrocarbylene group having 1 to 40 carbon atoms and optionally containing a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom. Specific examples of the hydrocarbylene group represented by X^{L3}, having 1 to 40 carbon atoms and optionally containing a heteroatom, include X^{L}-0 to X^{L}-58, which have been shown as specific examples of the hydrocarbylene group represented by X^{L1} and X^{L2}, having 1 to 40 carbon atoms and optionally containing a heteroatom, in the description of the formula (Z). Among them, X^{L}-0 to X^{L}-22, X^{L}-29 to X^{L}-34, and X^{L}-47 to X^{L}-58 are preferred.

For the rigidity of the resulting polymer, X^{L3} is preferably a single bond.

In the general formula (d1), Q¹ and Q² are each independently a hydrogen atom, a fluorine atom, or a fluorinated saturated hydrocarbyl group having 1 to 6 carbon atoms. The fluorinated saturated hydrocarbyl group having 1 to 6 carbon atoms is preferably a trifluoromethyl group.

In the general formula (d1), Q³ and Q⁴ are each independently a fluorine atom or a fluorinated saturated hydrocarbyl group having 1 to 6 carbon atoms. The fluorinated saturated hydrocarbyl group having 1 to 6 carbon atoms is preferably a trifluoromethyl group. Q³ and Q⁴ are more preferably each a fluorine atom.

Preferred specific examples of the partial structure represented by—[C(Q¹)(Q²)]_{d6}-C(Q³)(Q⁴)-SO₃⁻ in the general formula (d1) are those shown below, but are not limited thereto. In the following formulae, * indicates bonding to L^{C}.

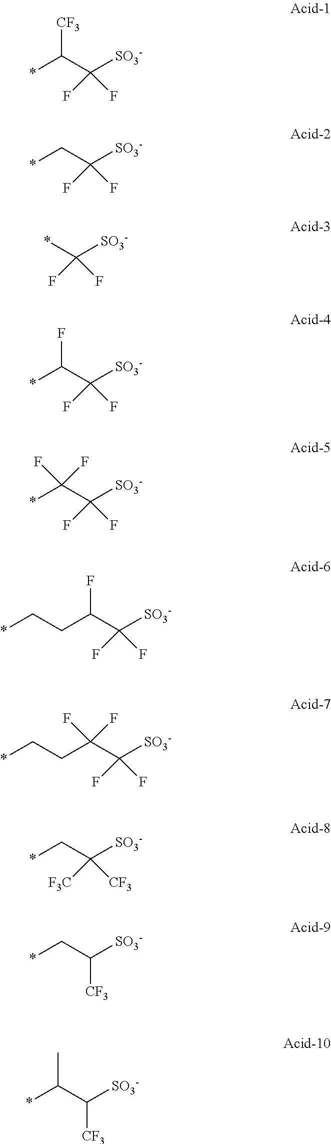

Among these, Acid-1 to Acid-7 are preferred, and Acid-1 to Acid-3, Acid-6, and Acid-7 are more preferred.

The anion represented by the general formula (d1) is preferably one represented by the following general formula (d1-1):

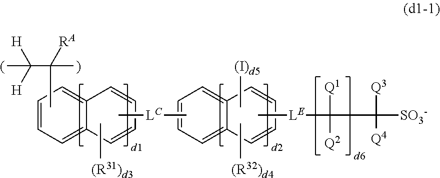

wherein d1 to d6, R^{A}, R³¹, R³², L^{C}, L^{E}, and Q¹ to Q⁴ are as specified above.

The anion represented by the general formula (d1-1) is preferably one represented by the following general formula (d1-2):

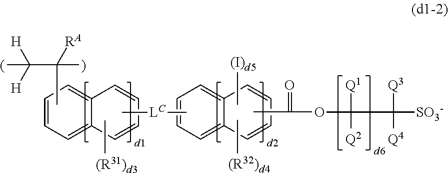

wherein d1 to d6, R^{A}, R³¹, R³², L^{c}, Q¹, and Q² are as specified above.

Specific examples of the anion represented by the general formula (d1) include, but are not limited to, those shown below. In the following formulae, R^{A} and Q¹ are as specified above, and Me is a methyl group. The bonding positions of substituents in each aromatic ring may be interchanged therein.

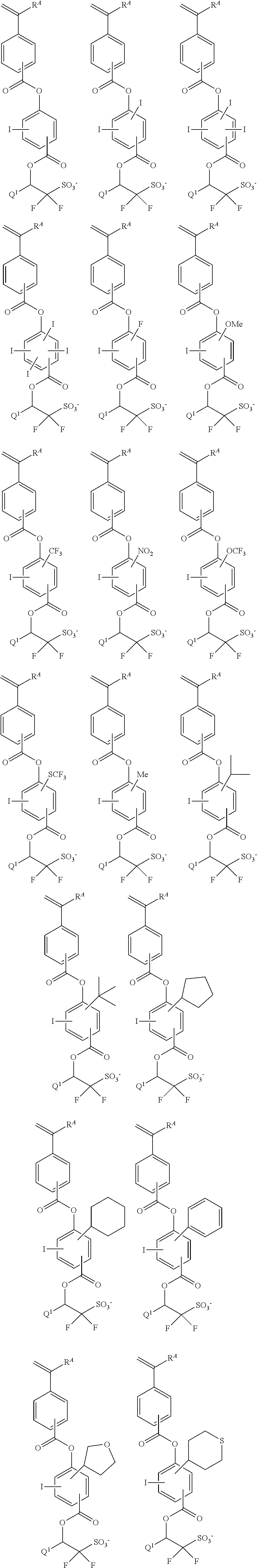

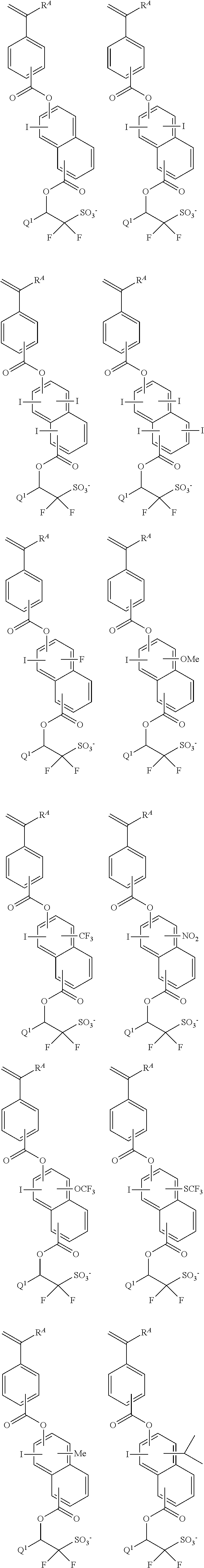

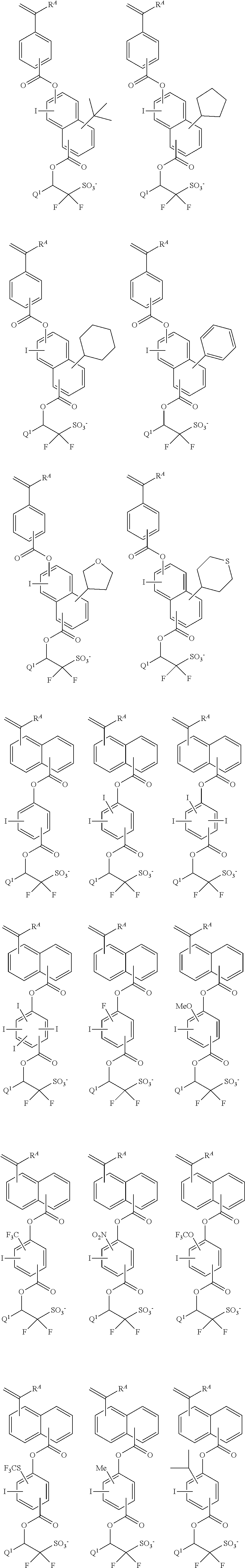

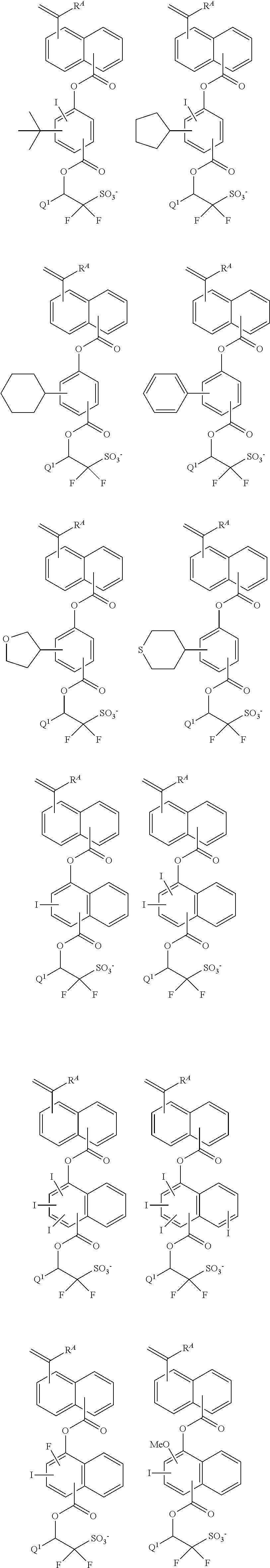

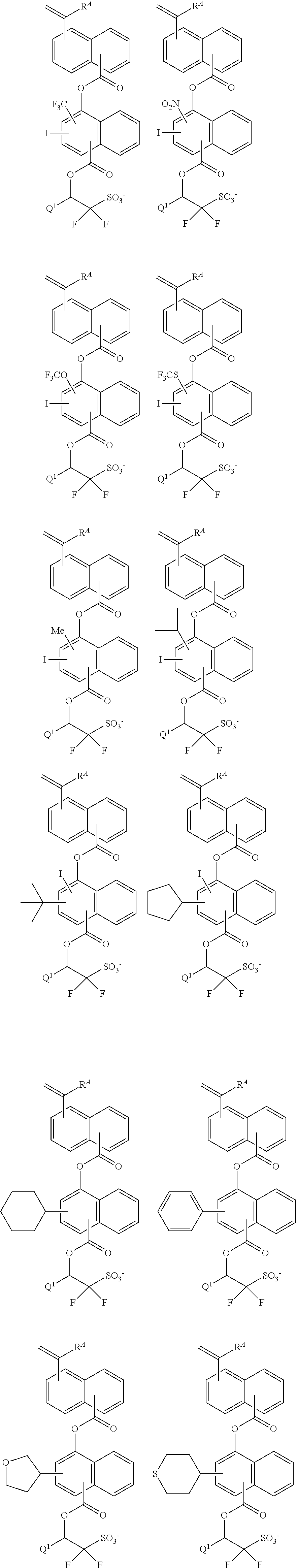

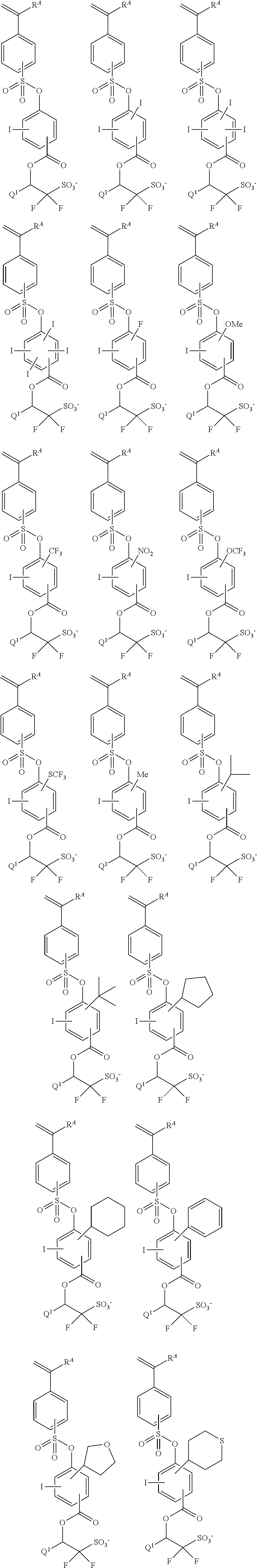

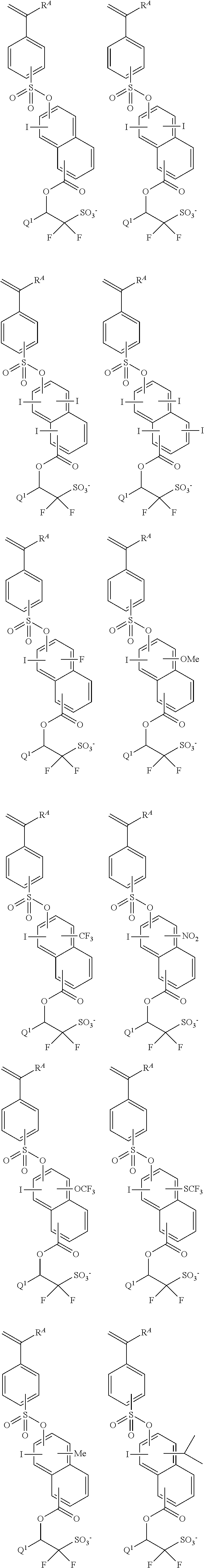

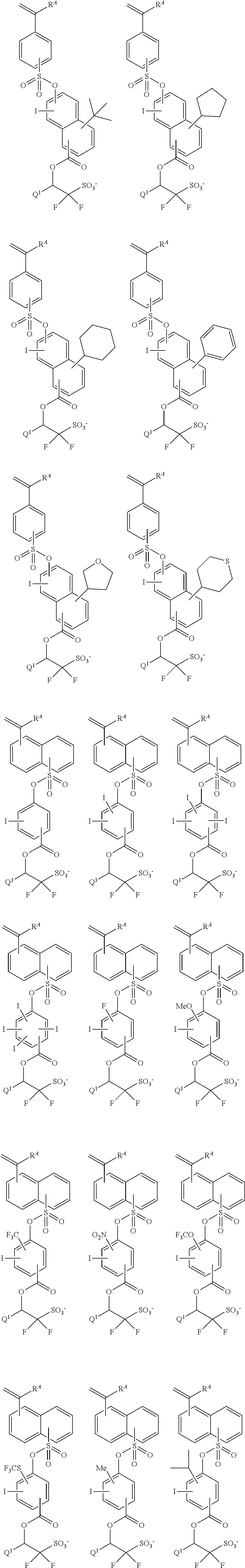

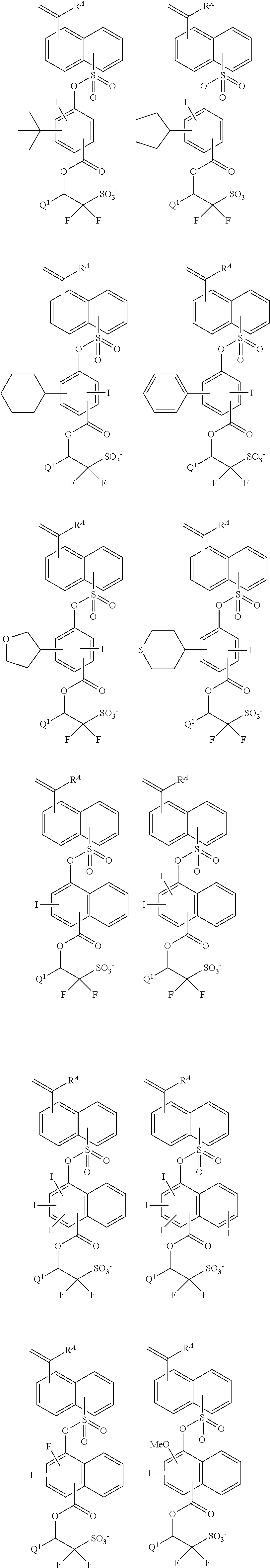

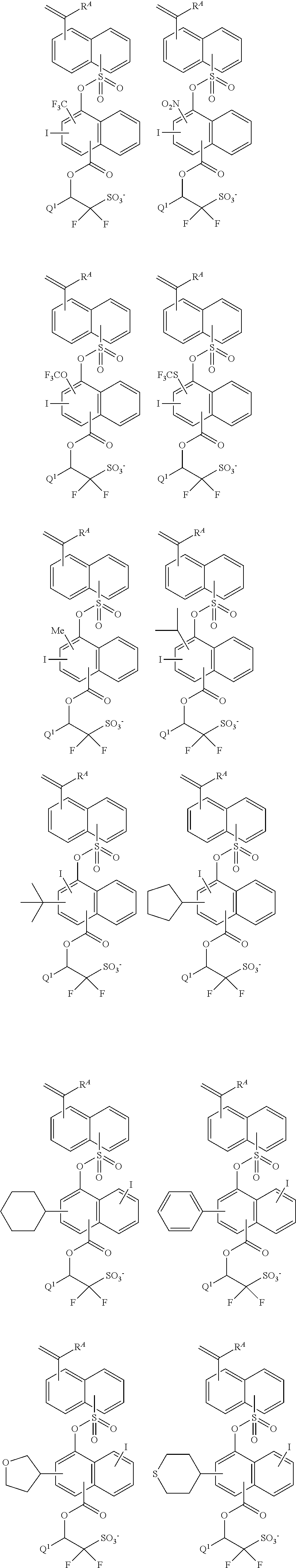

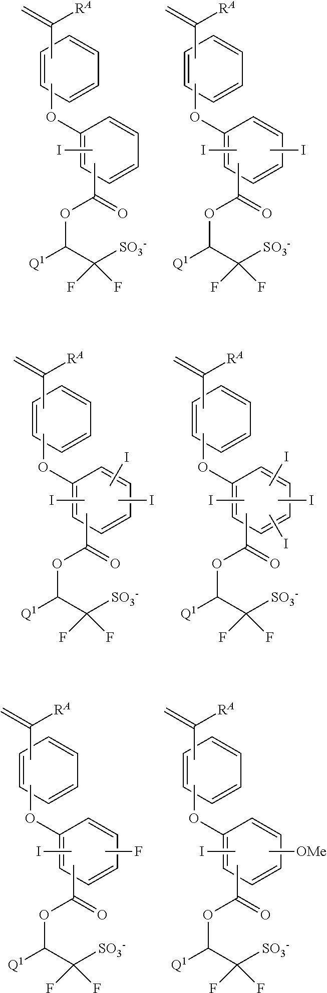

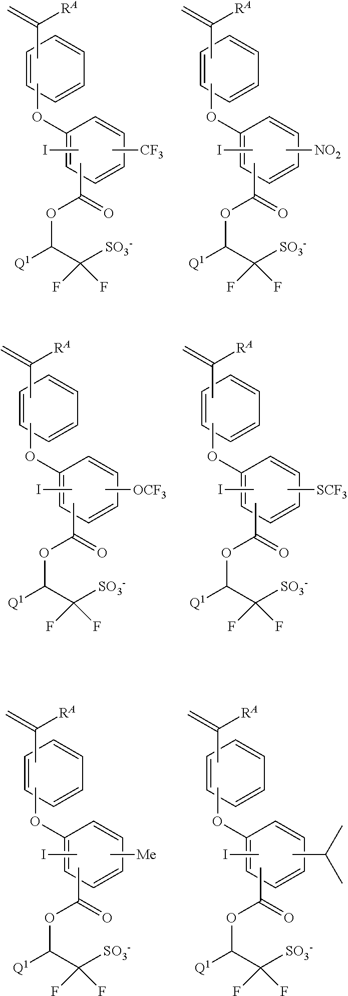

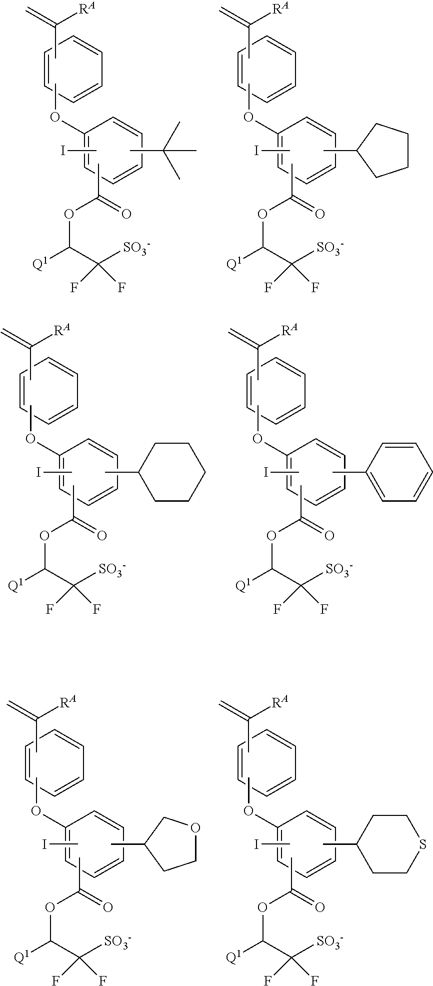

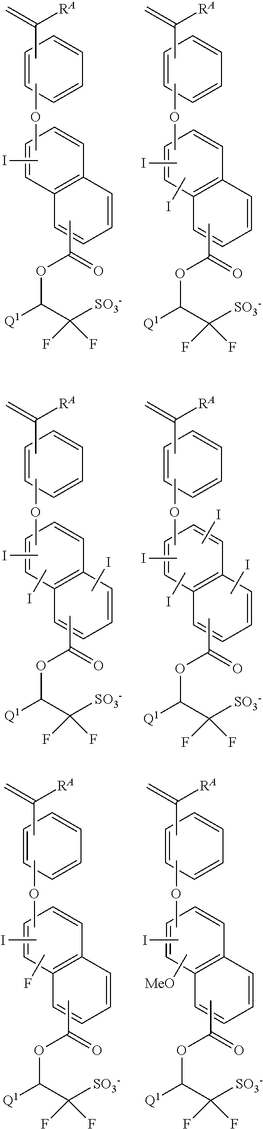

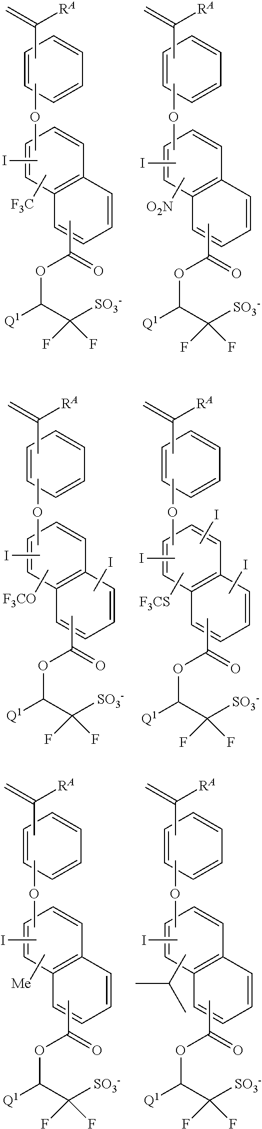

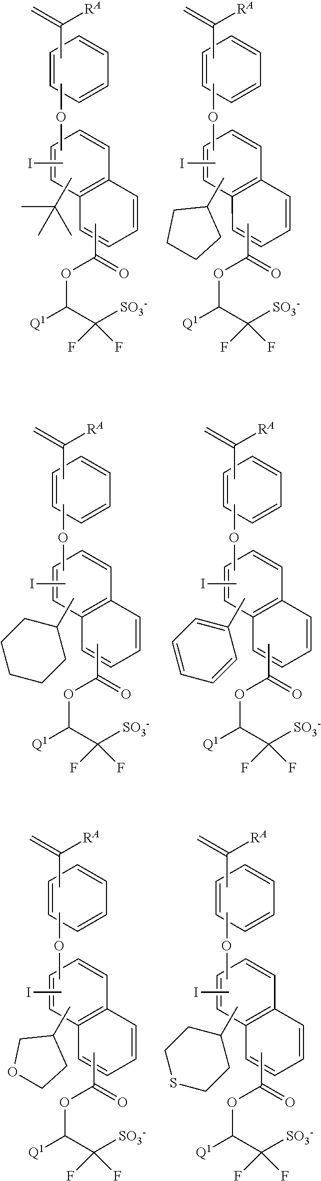

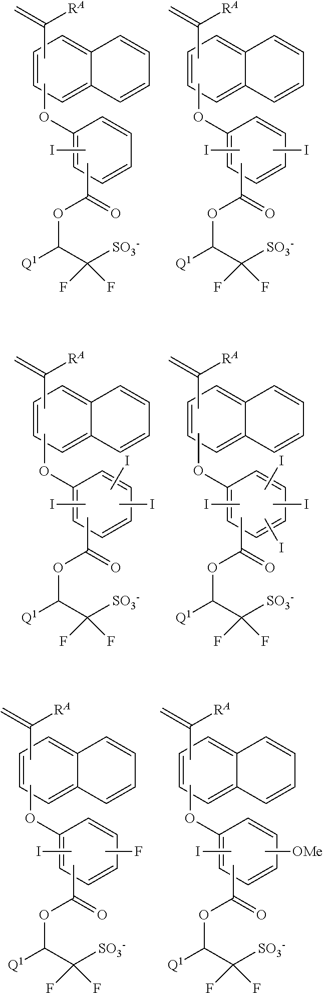

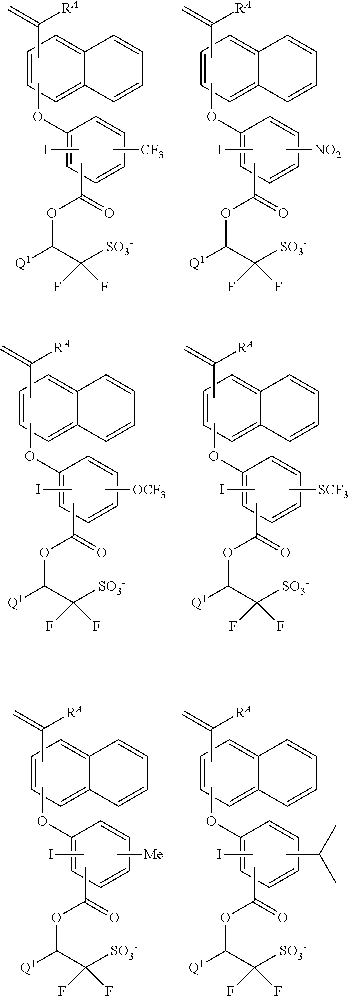

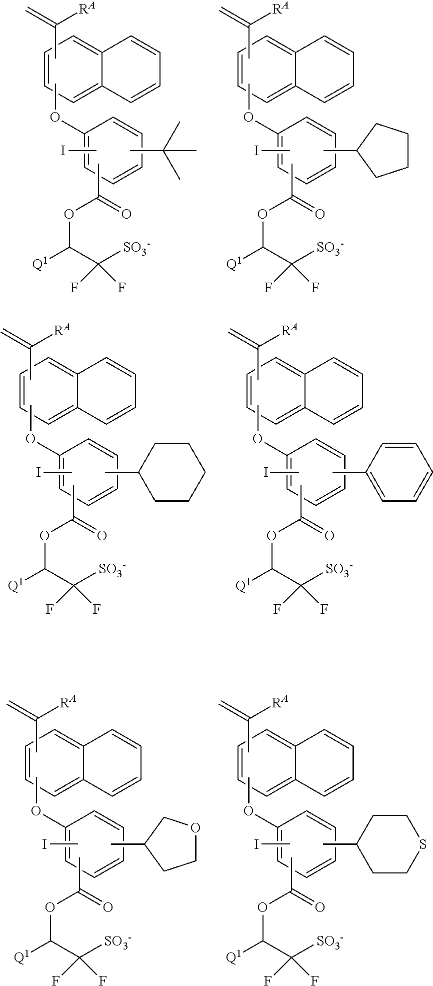

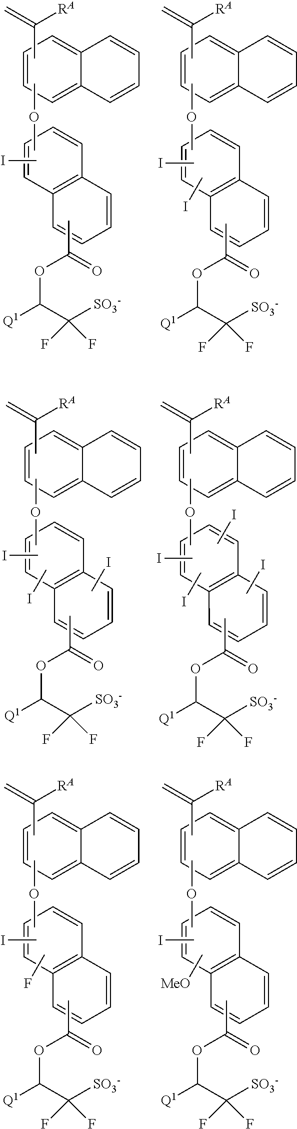

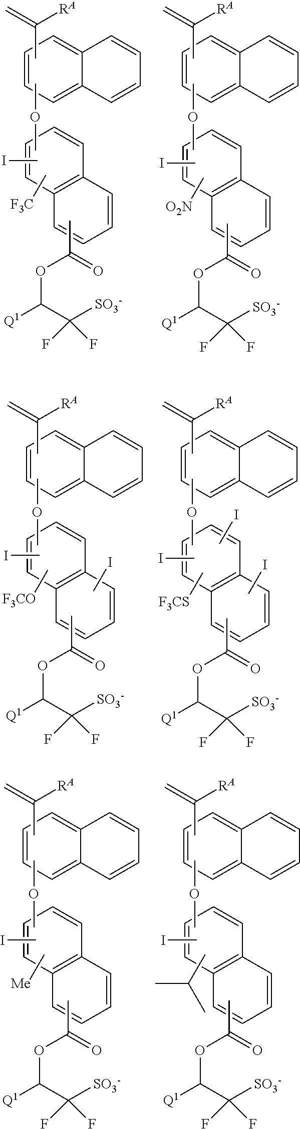

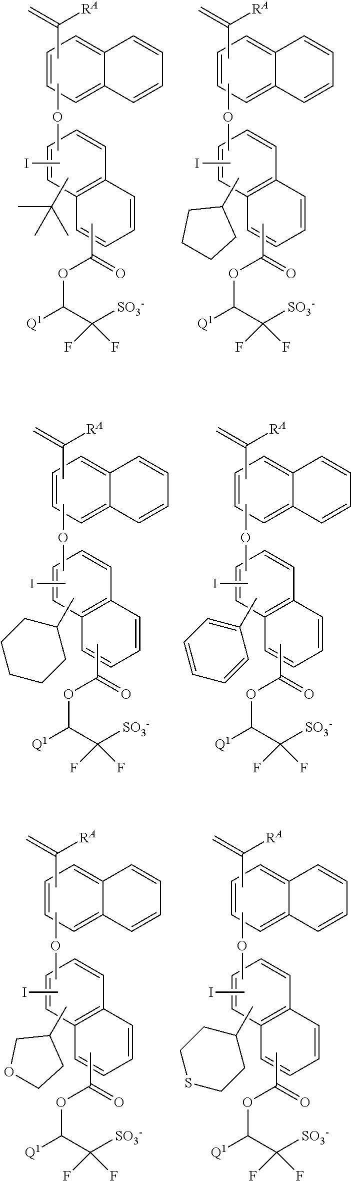

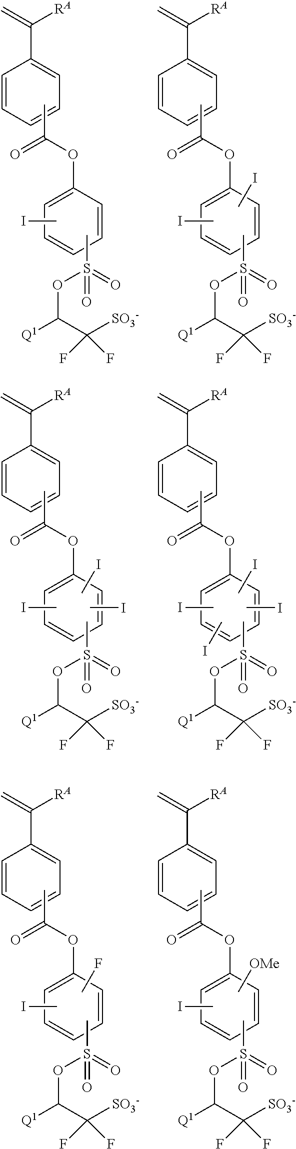

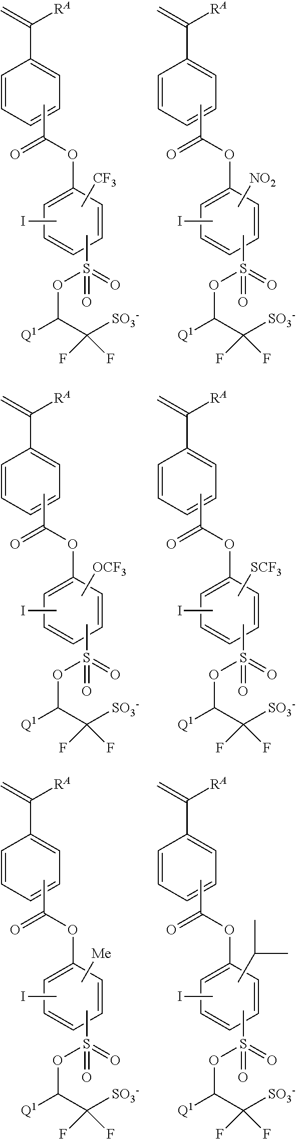

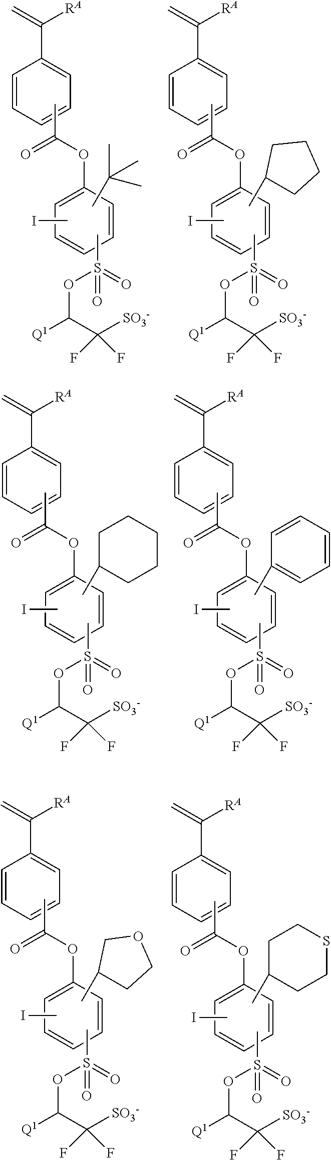

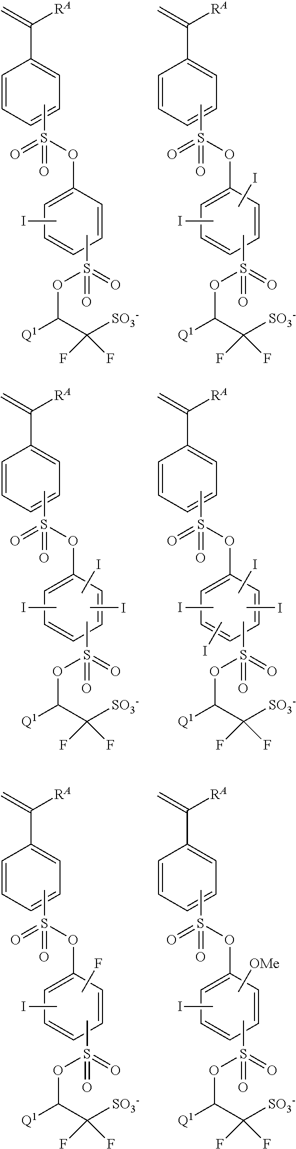

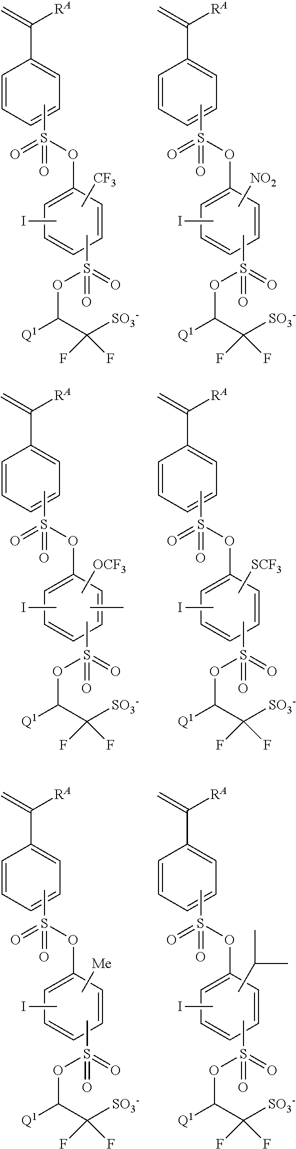

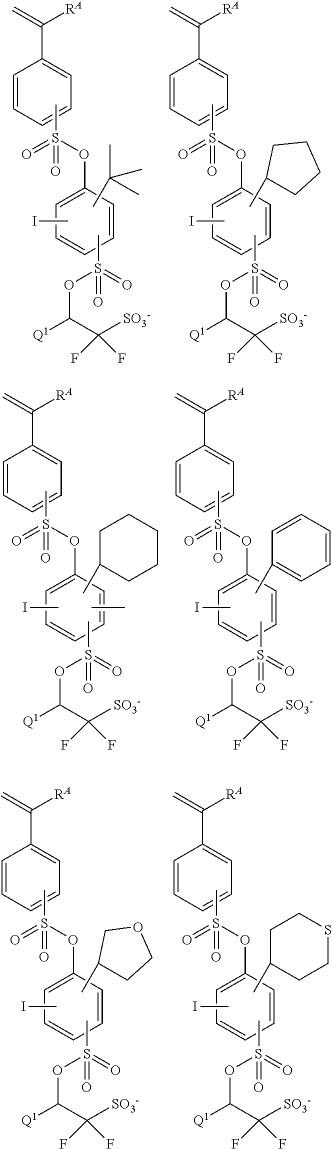

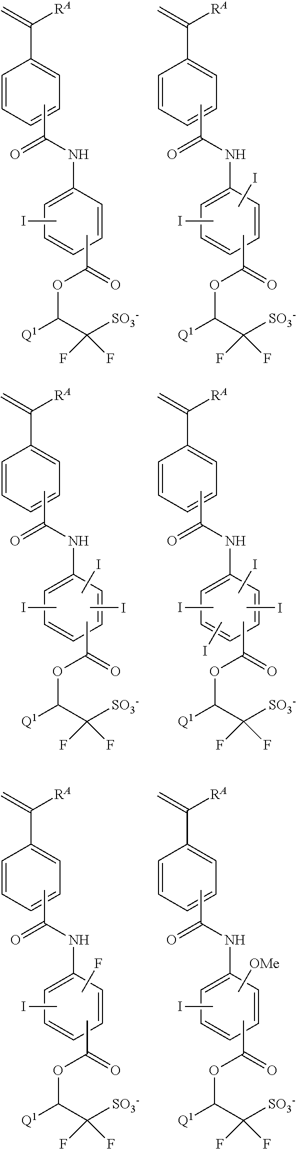

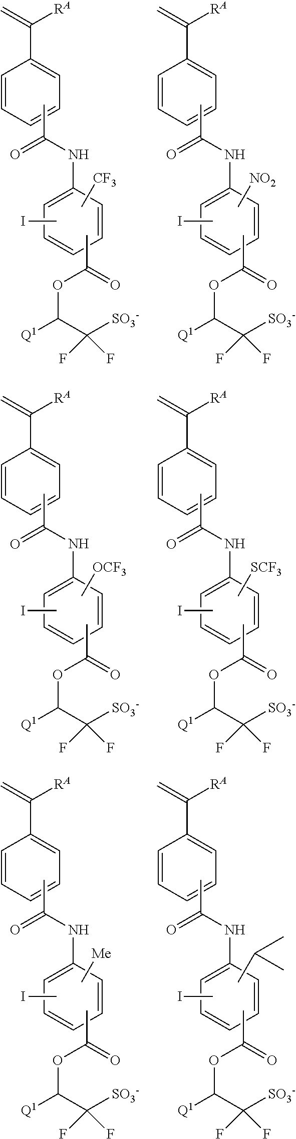

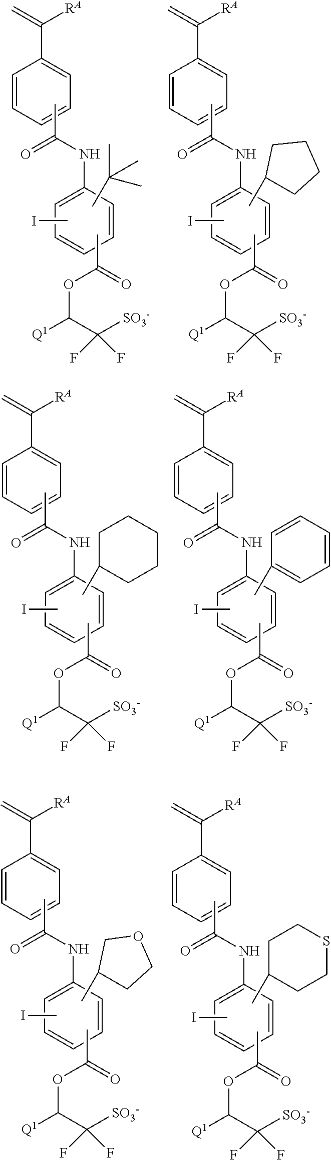

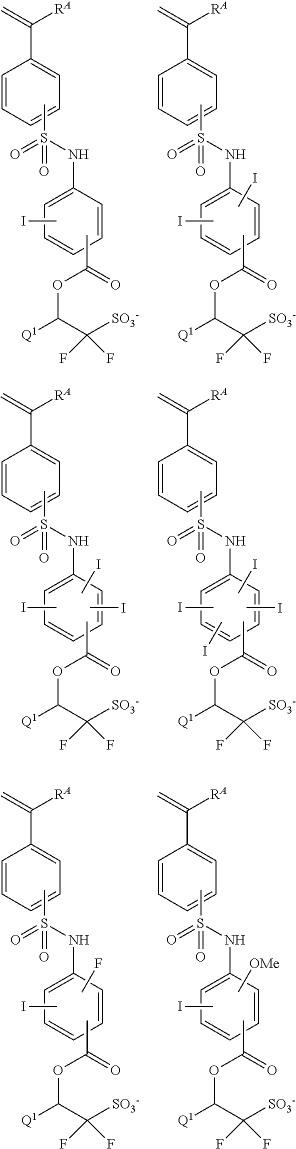

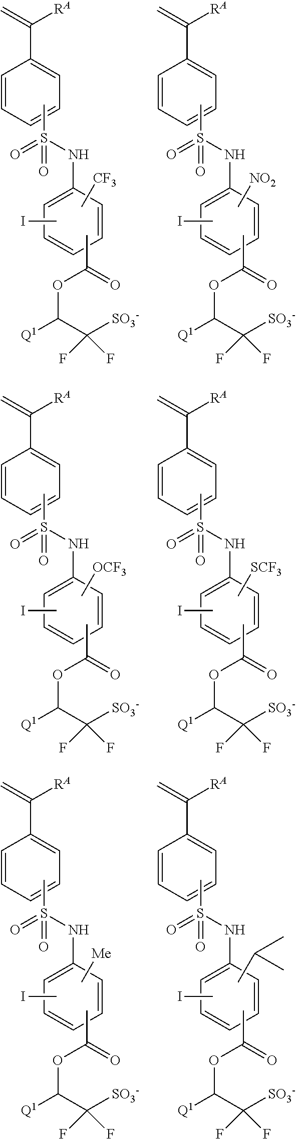

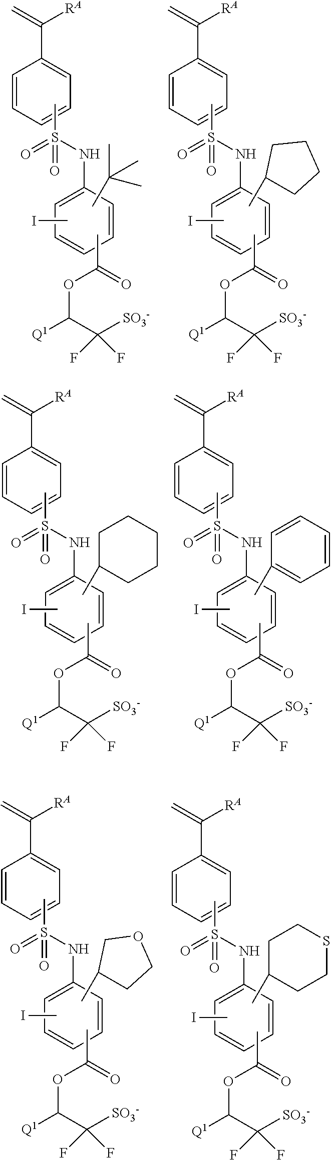

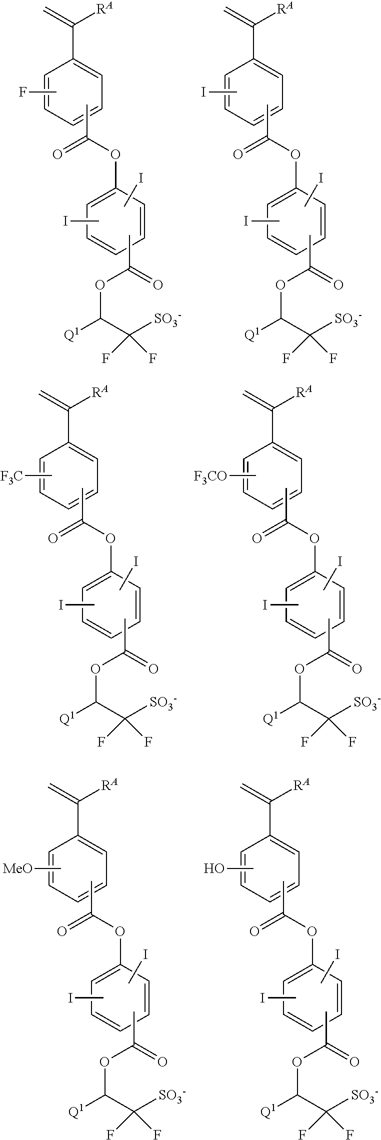

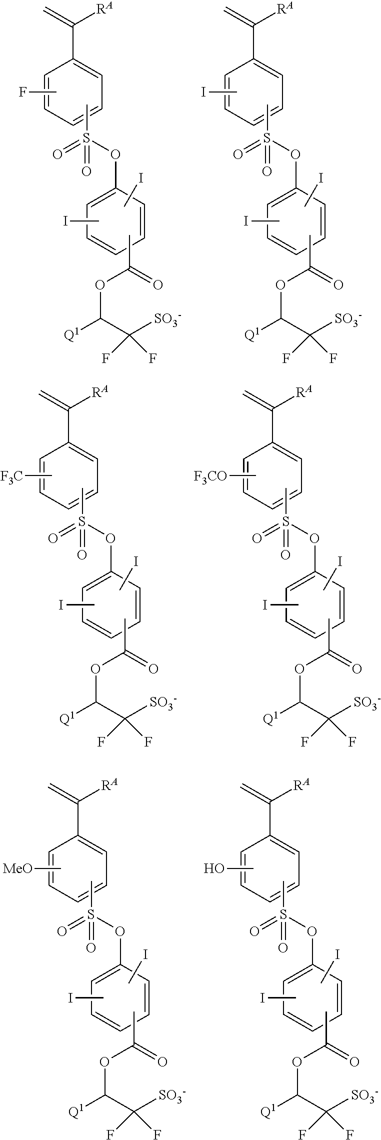

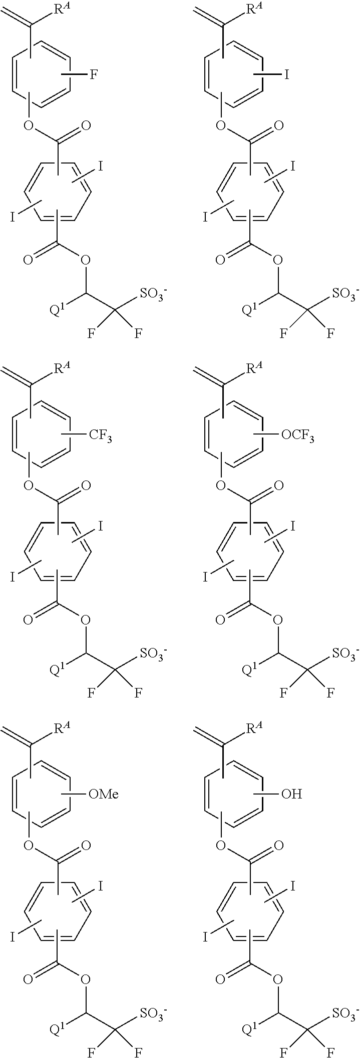

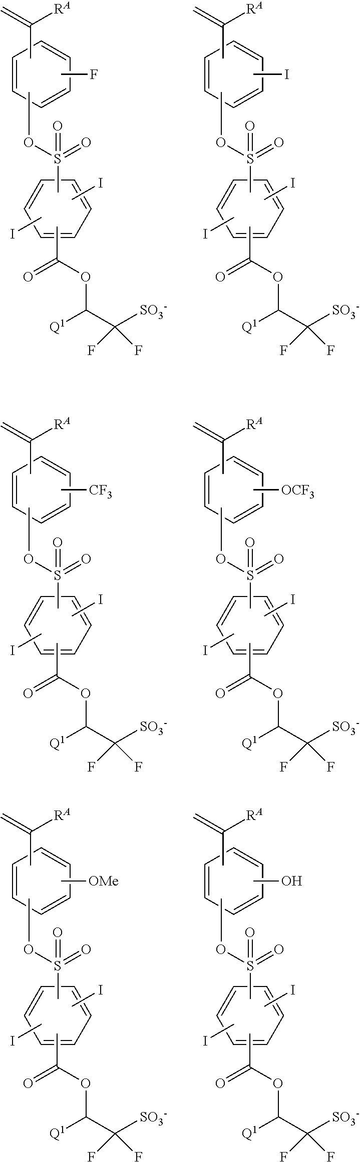

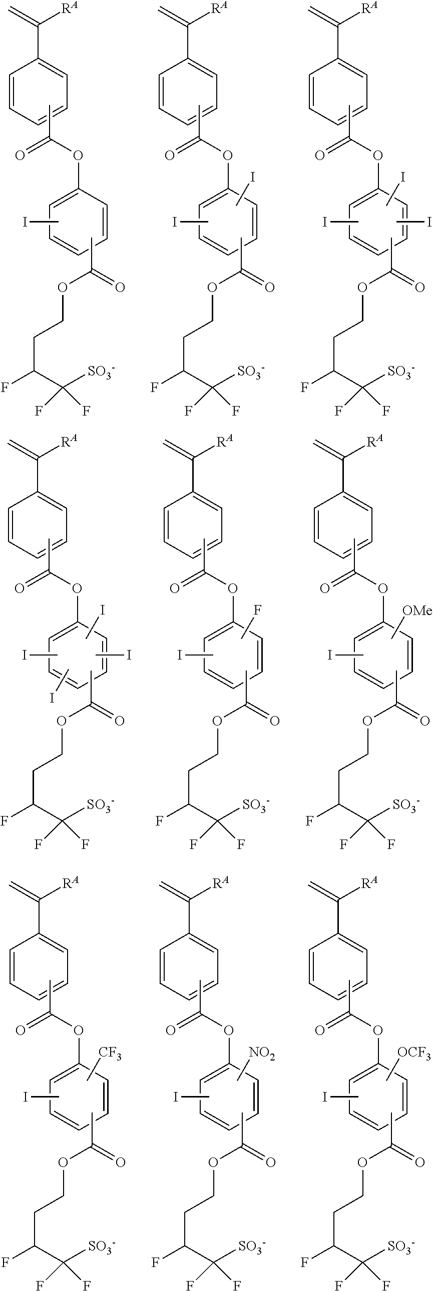

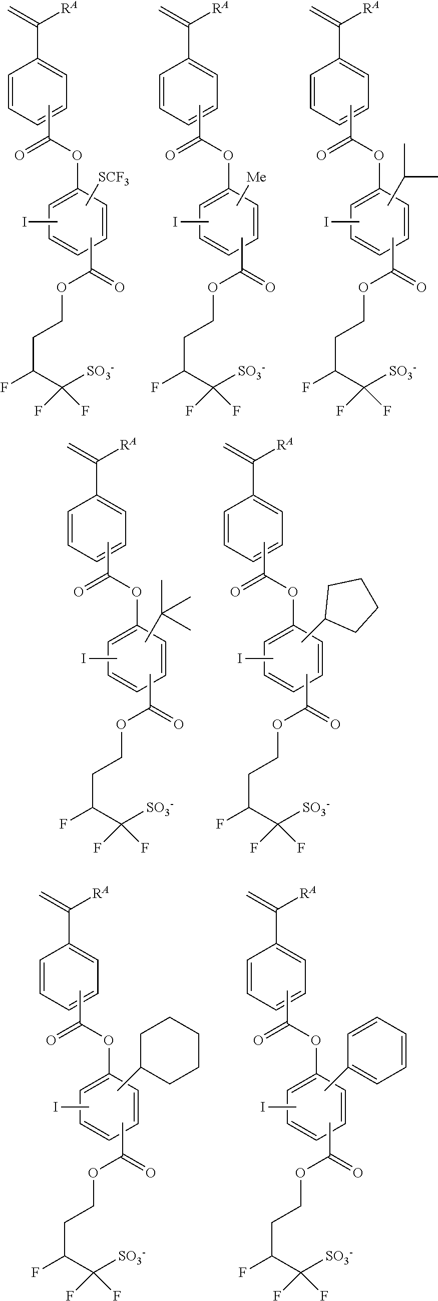

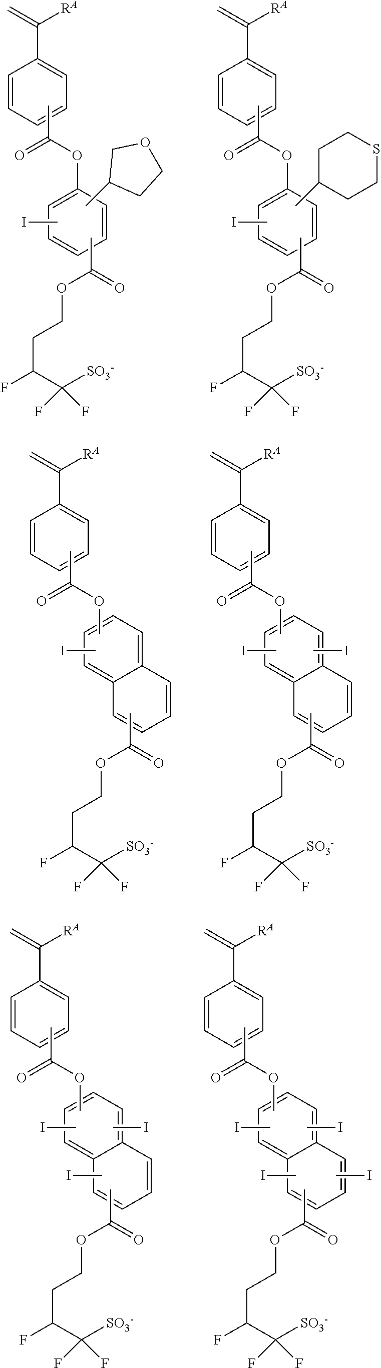

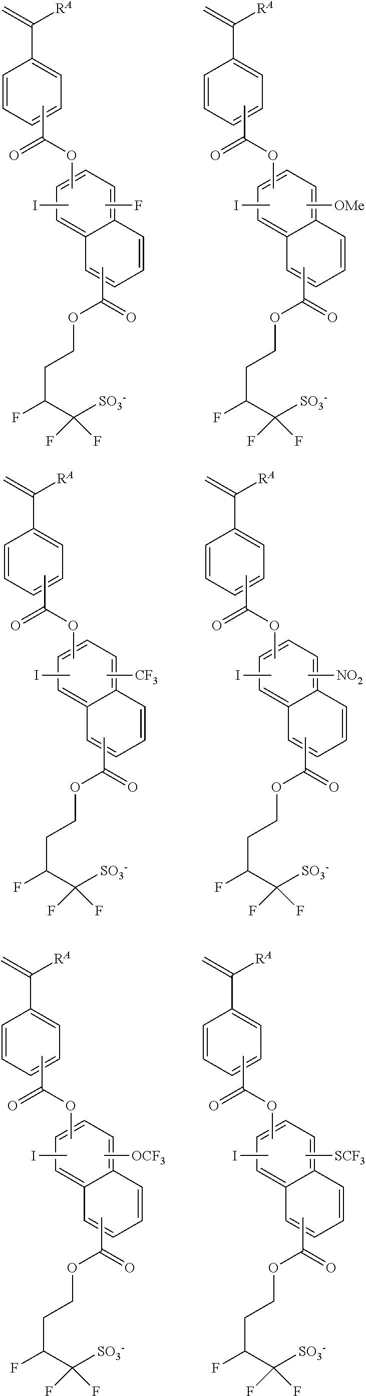

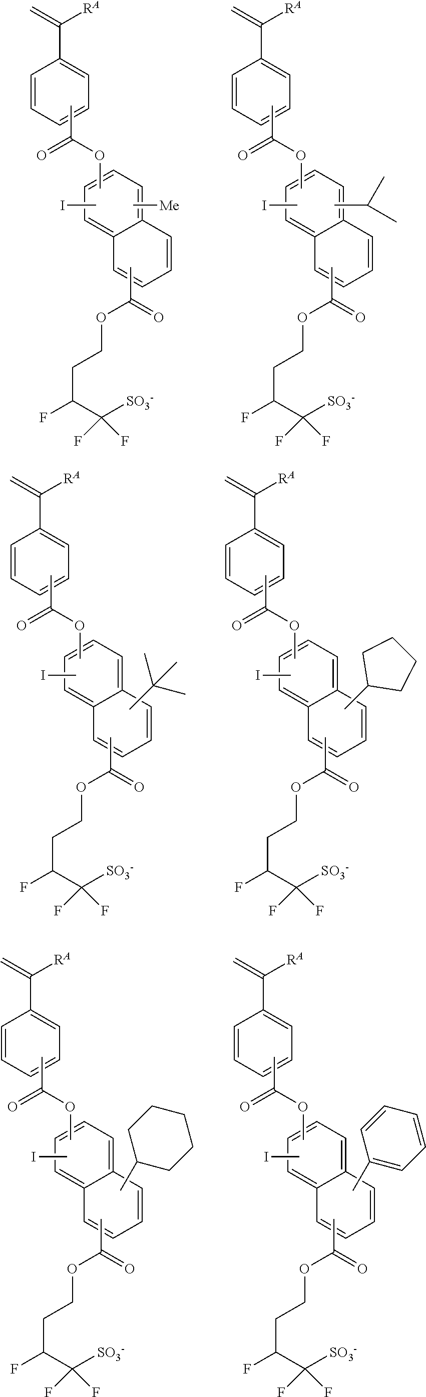

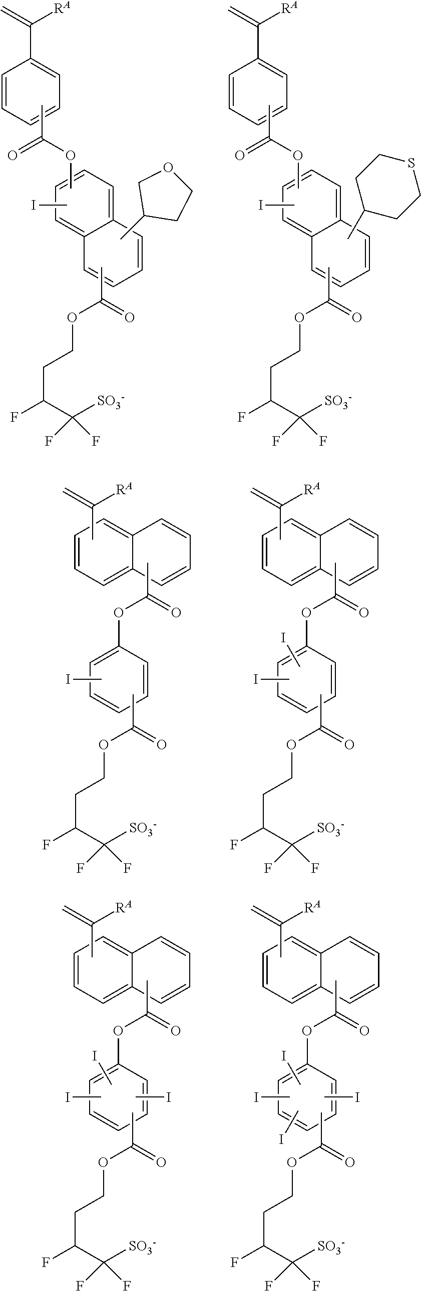

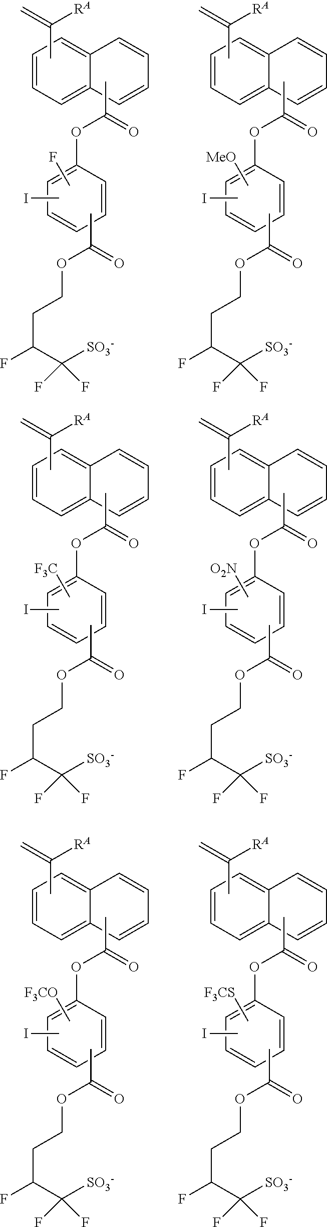

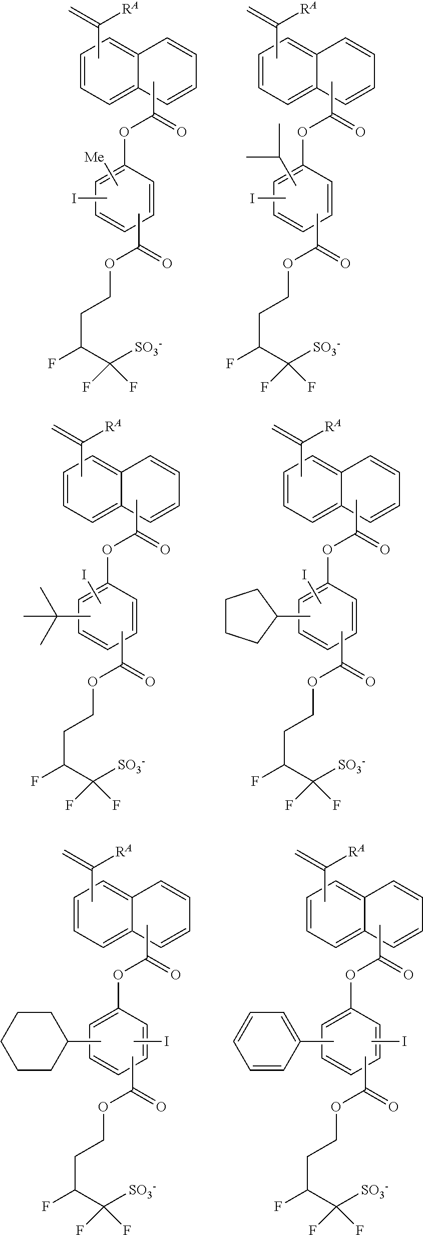

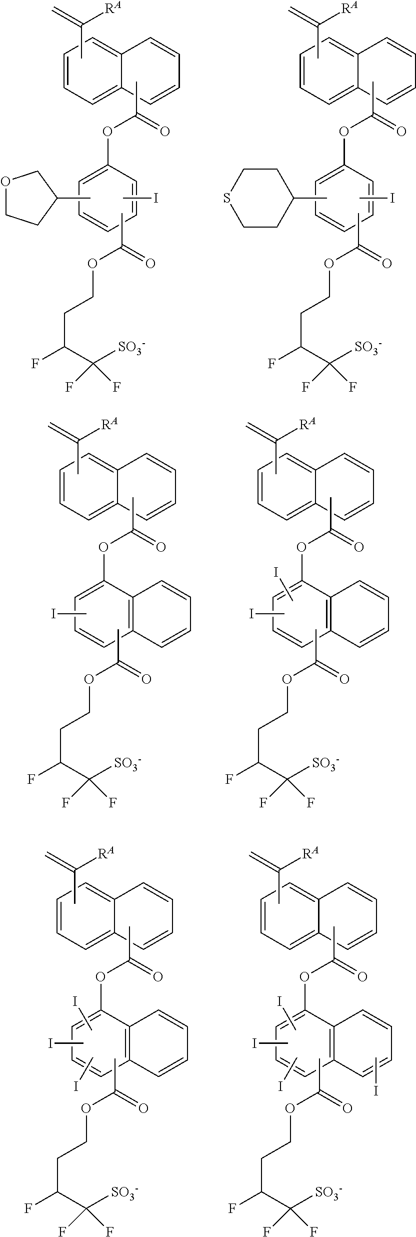

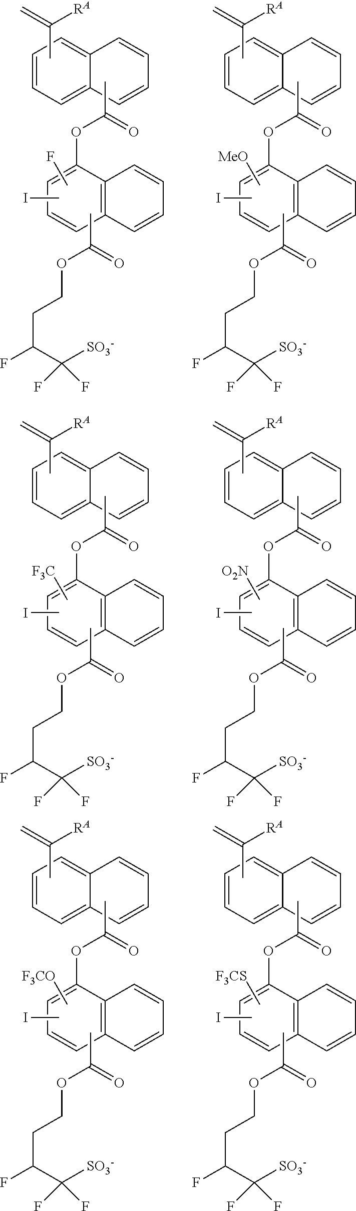

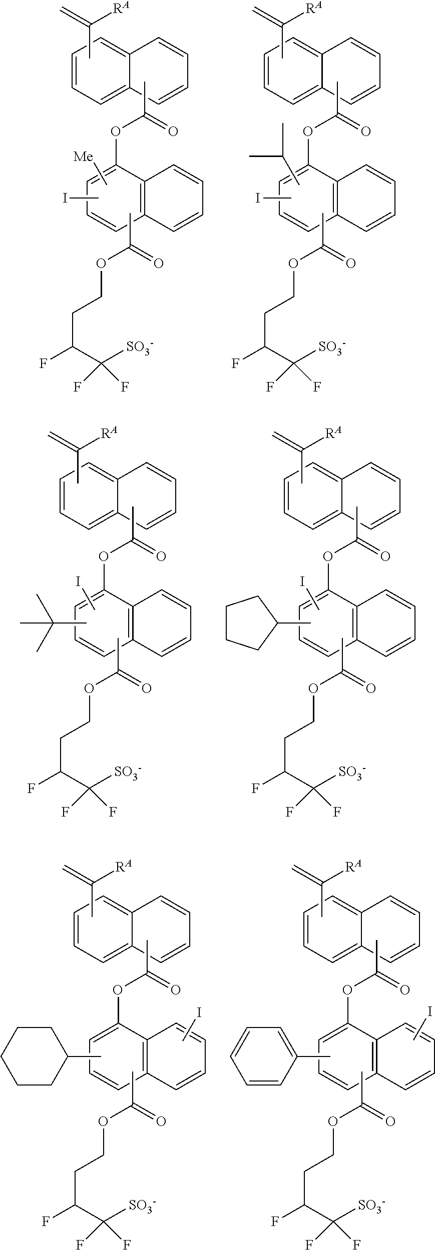

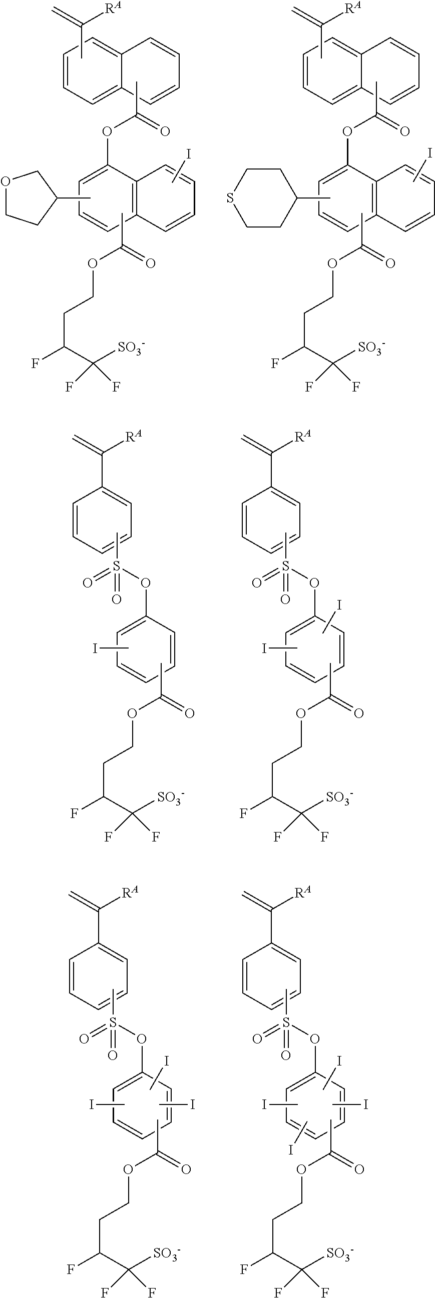

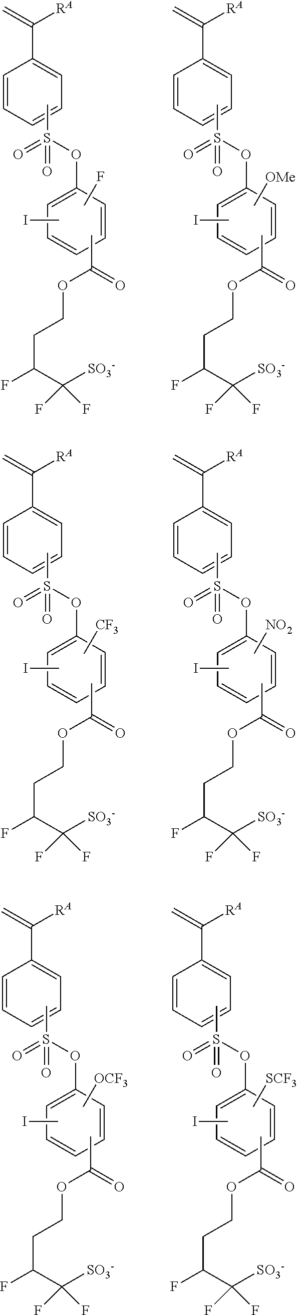

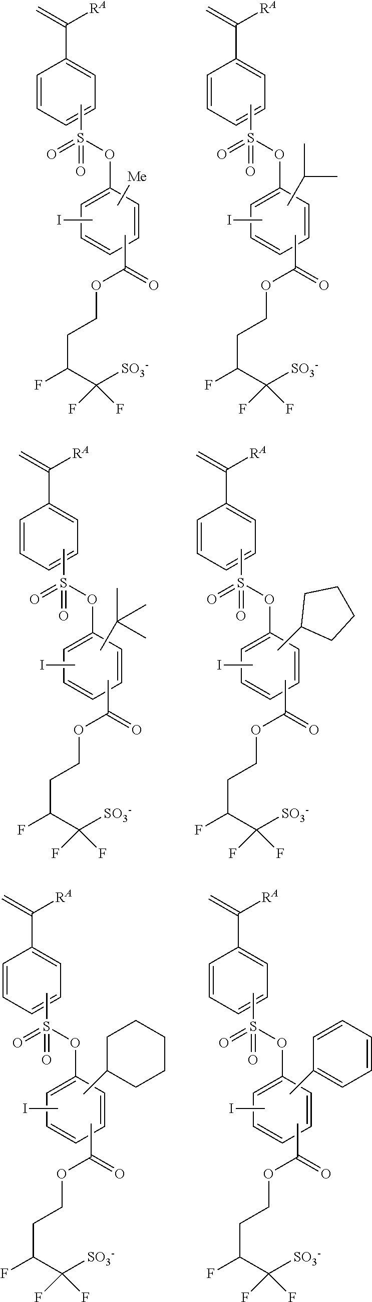

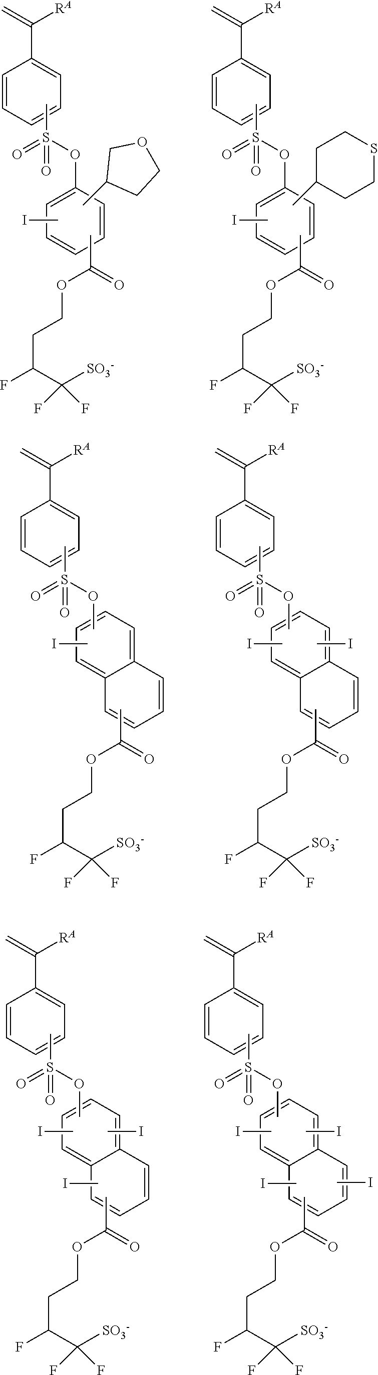

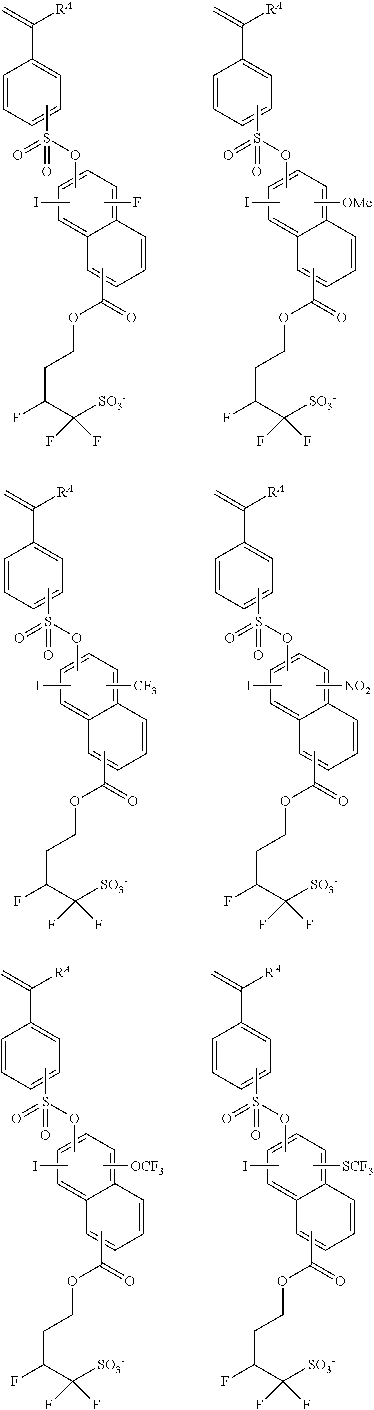

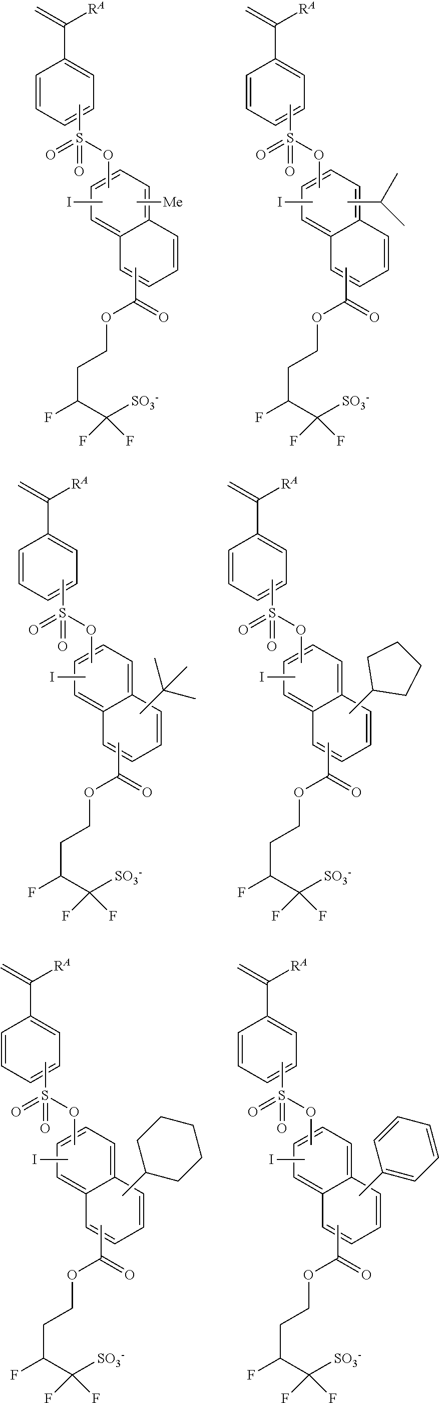

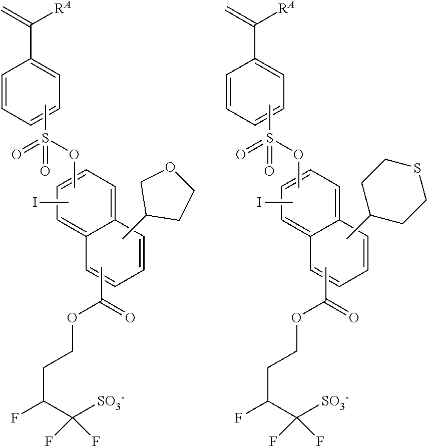

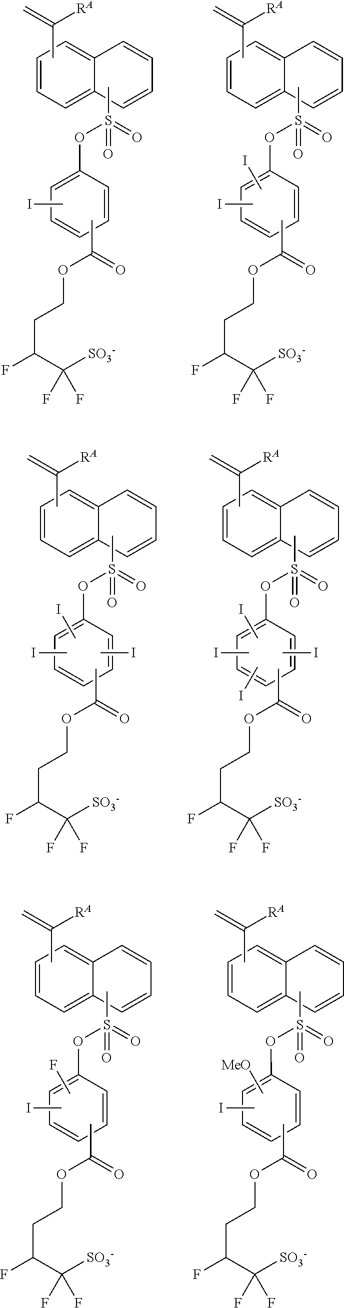

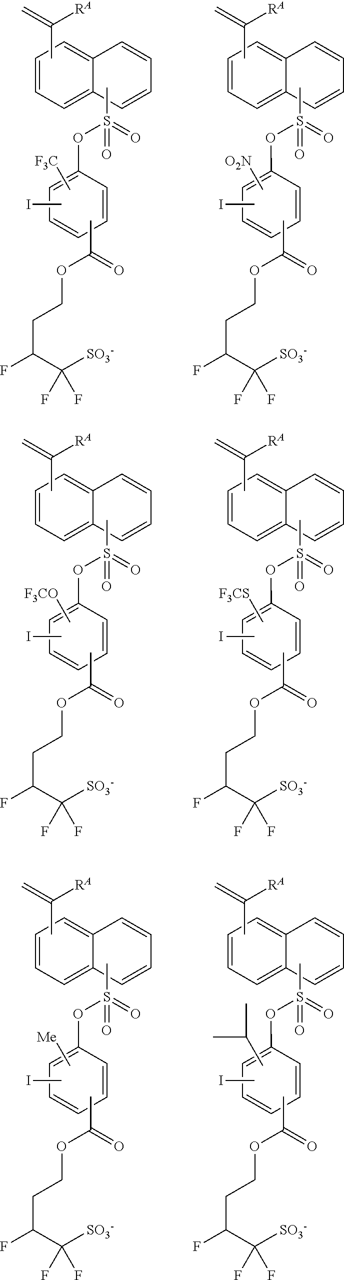

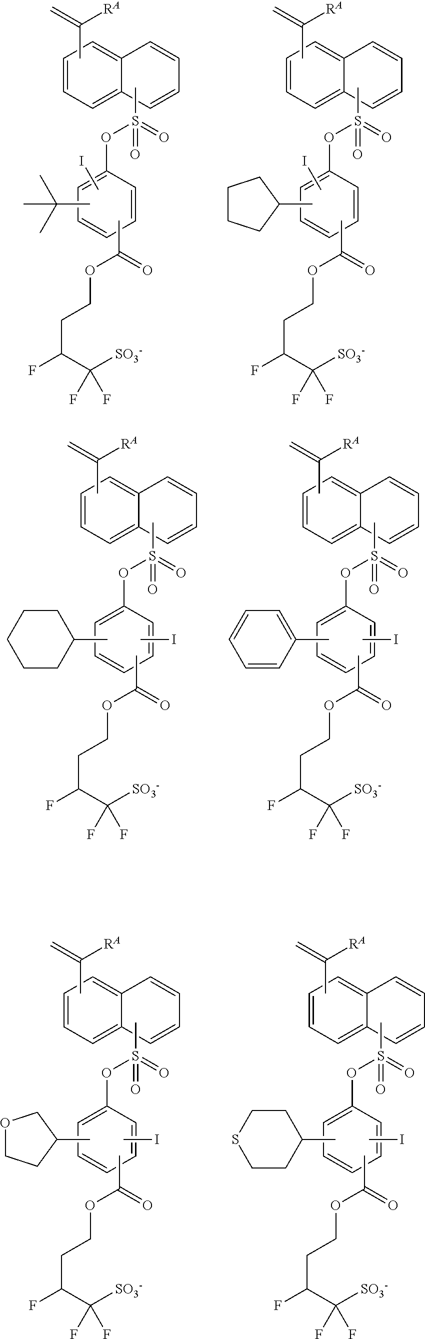

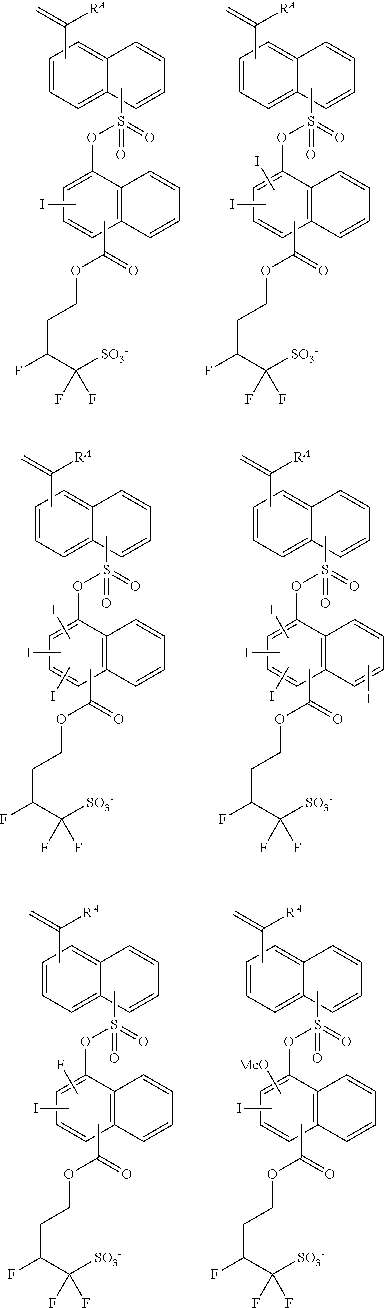

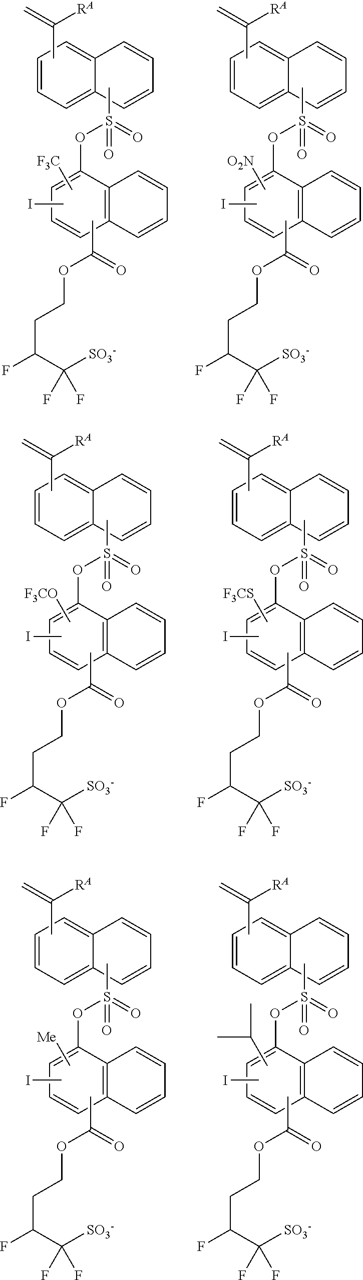

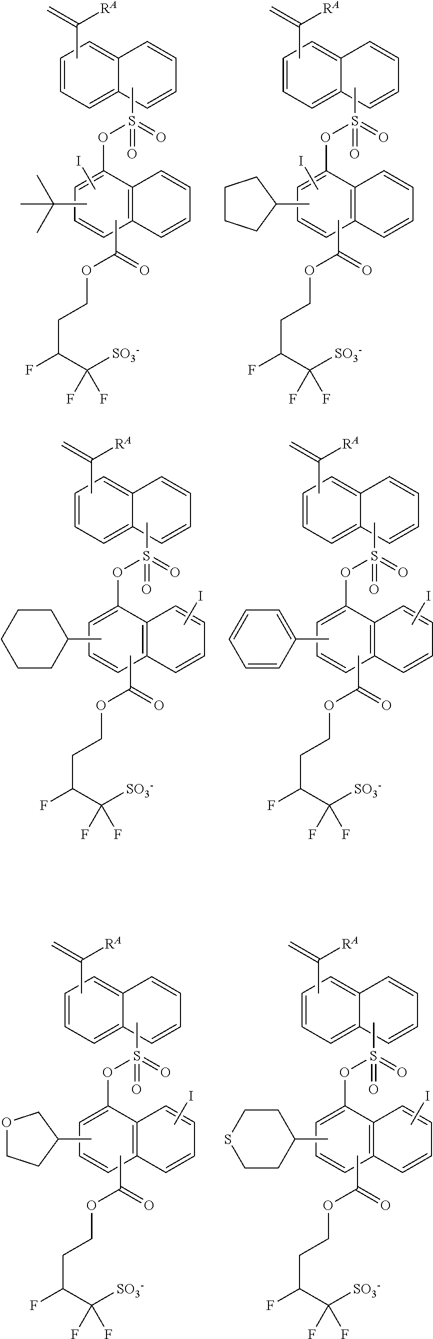

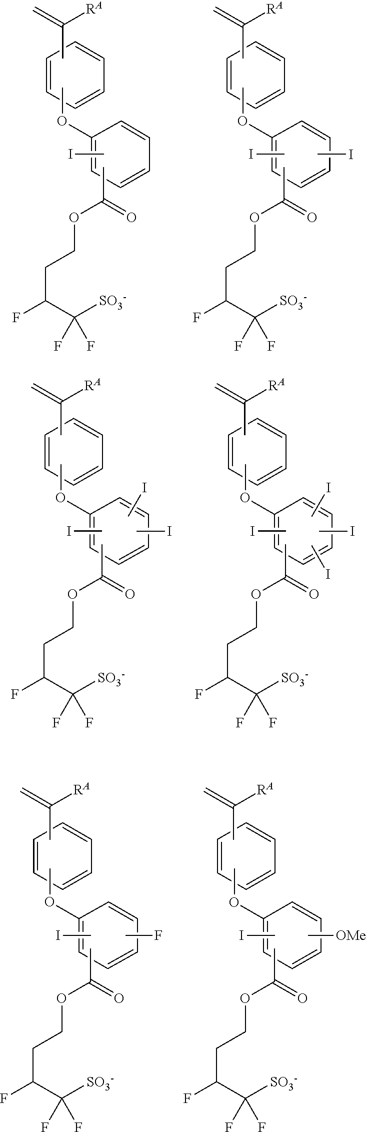

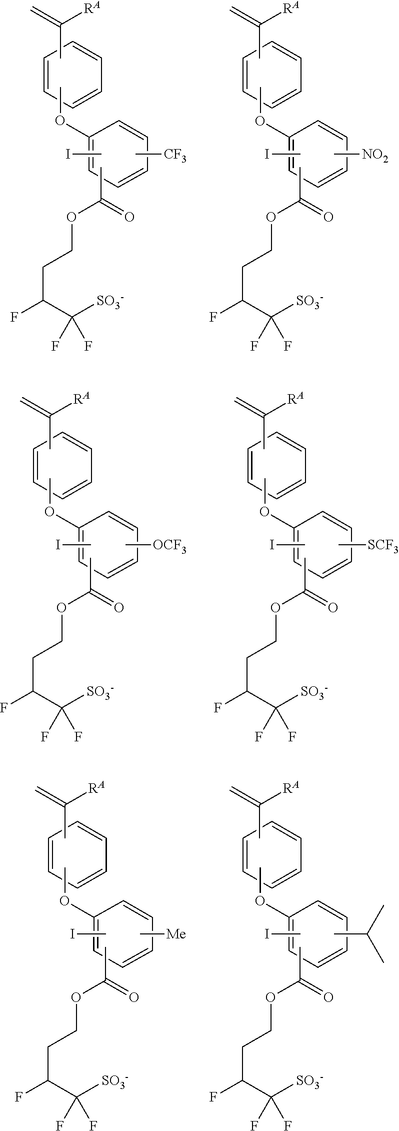

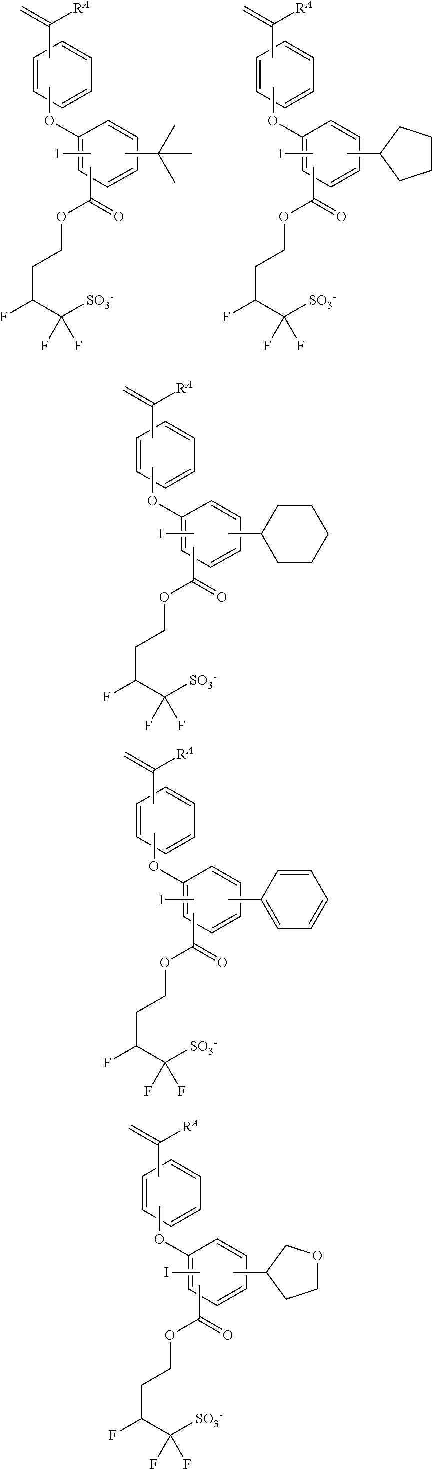

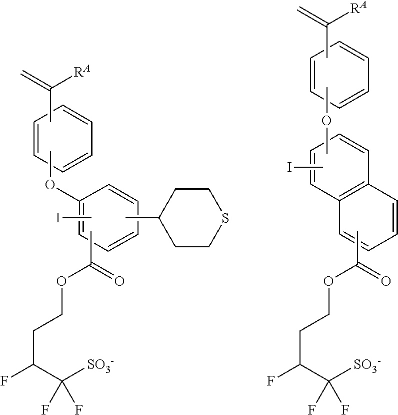

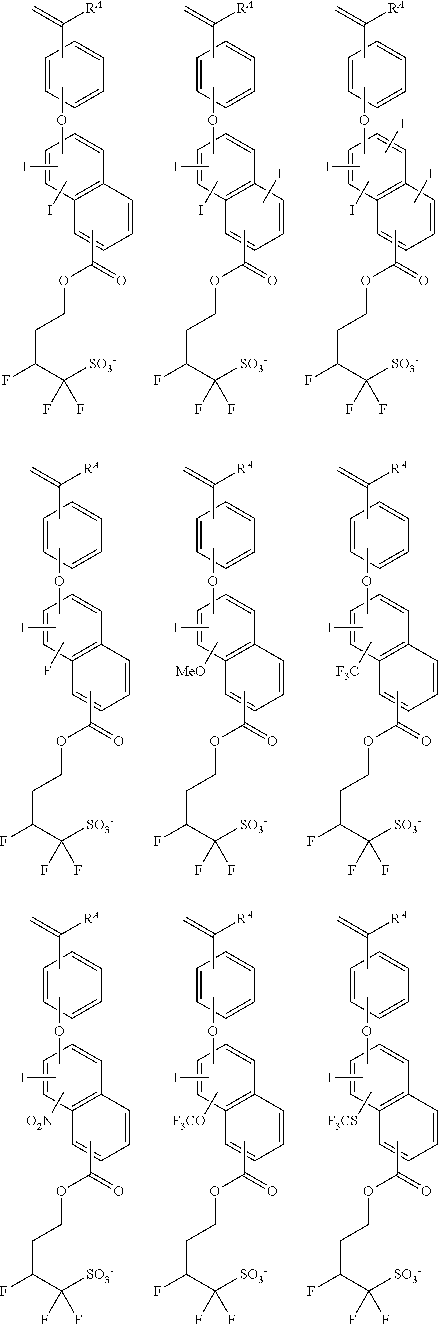

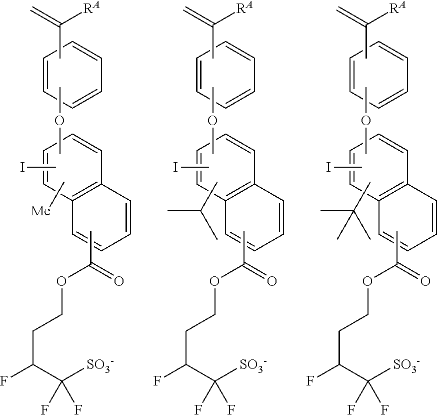

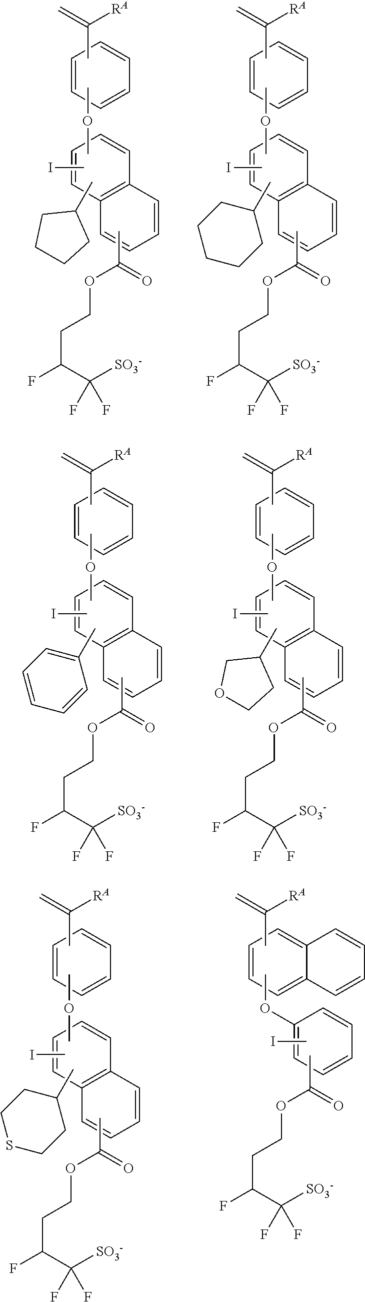

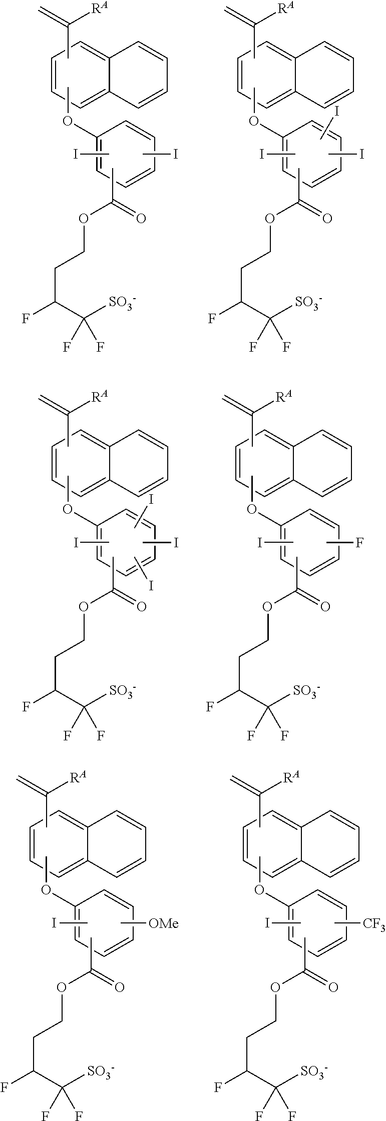

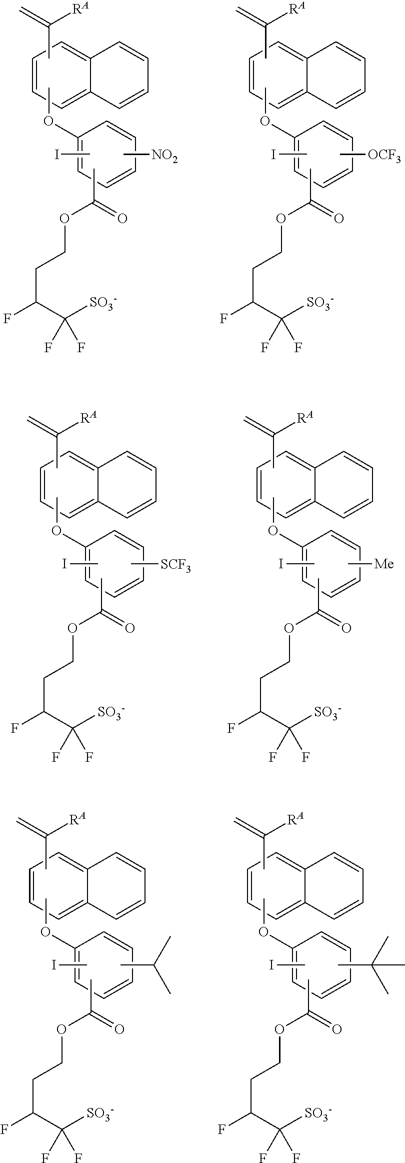

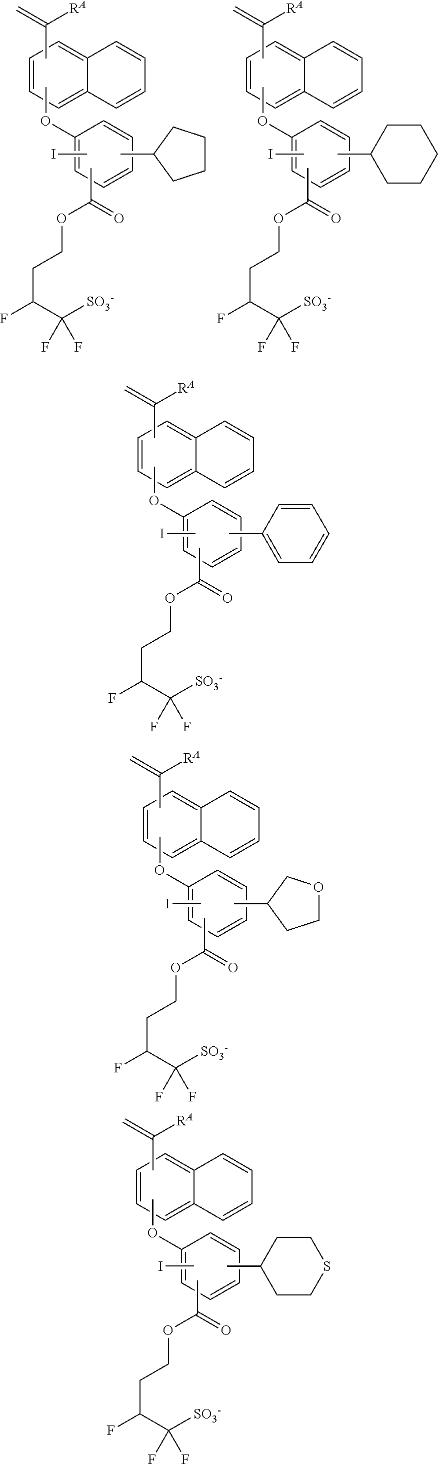

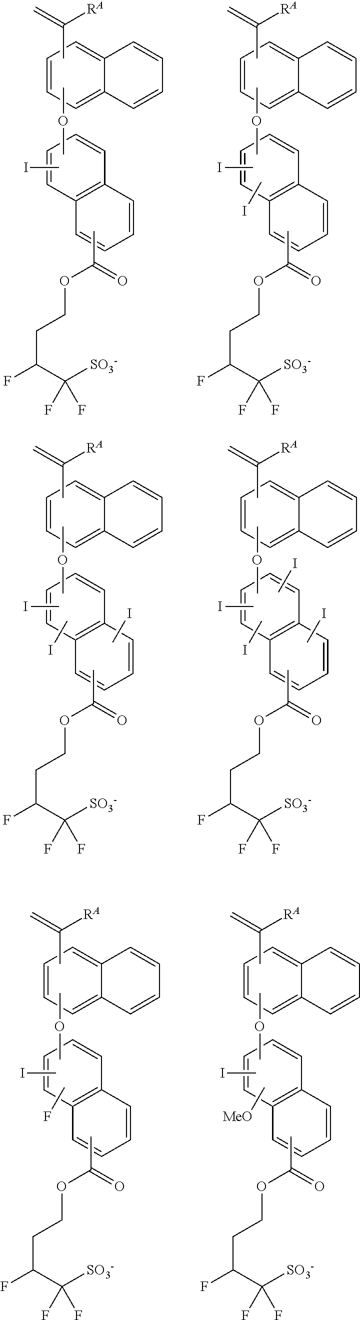

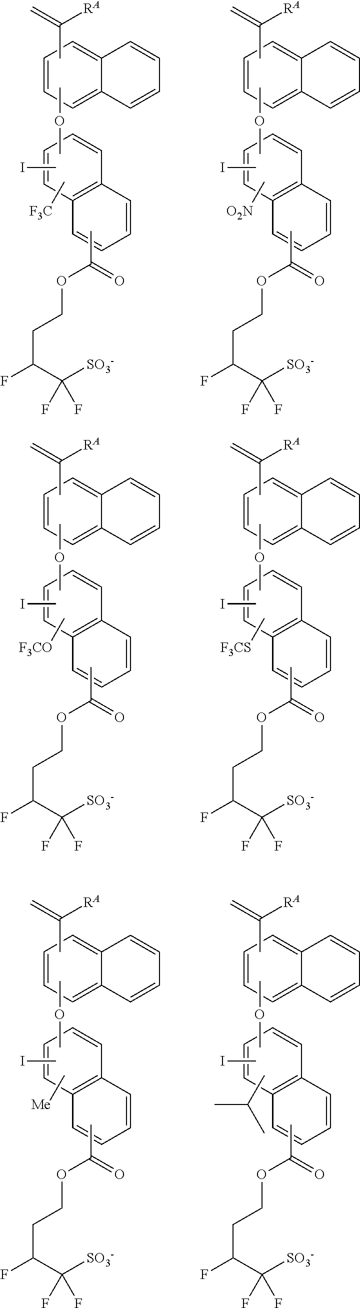

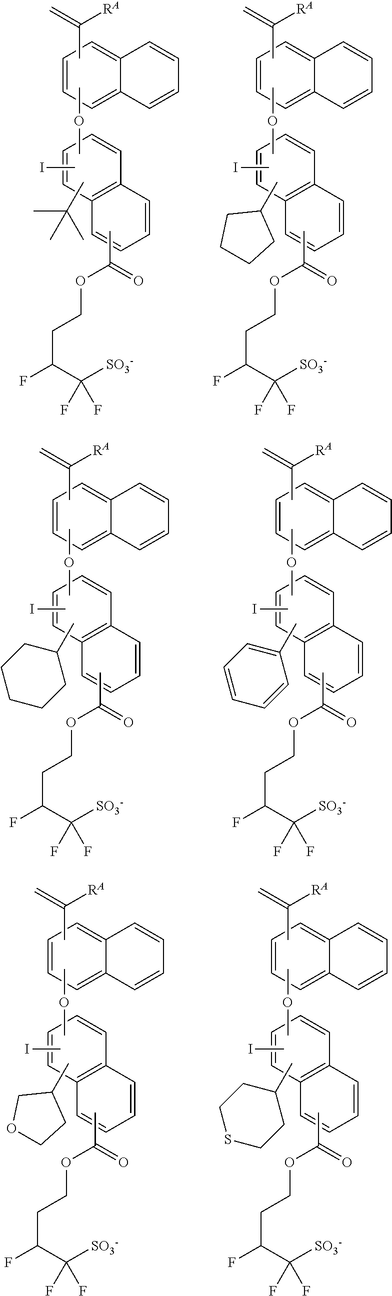

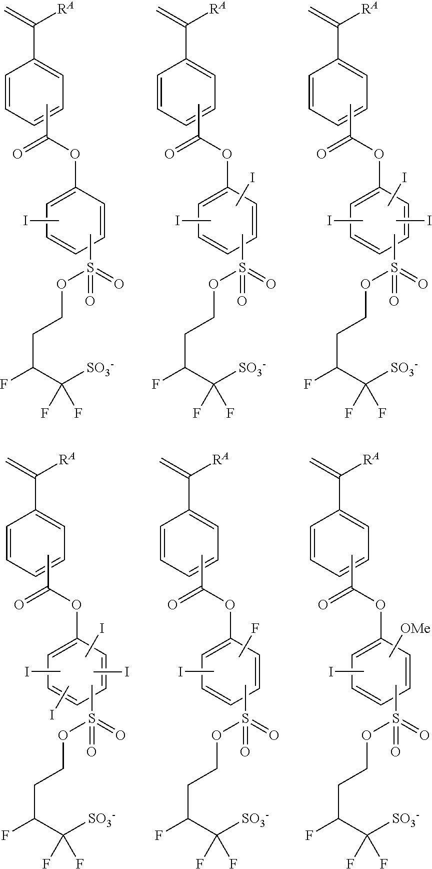

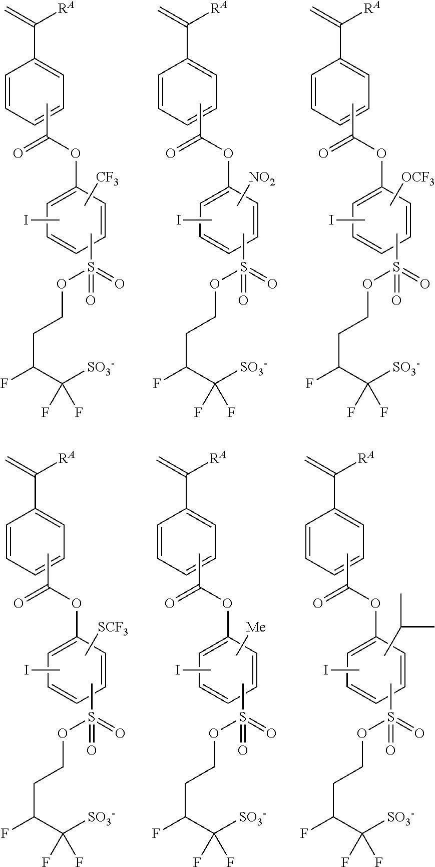

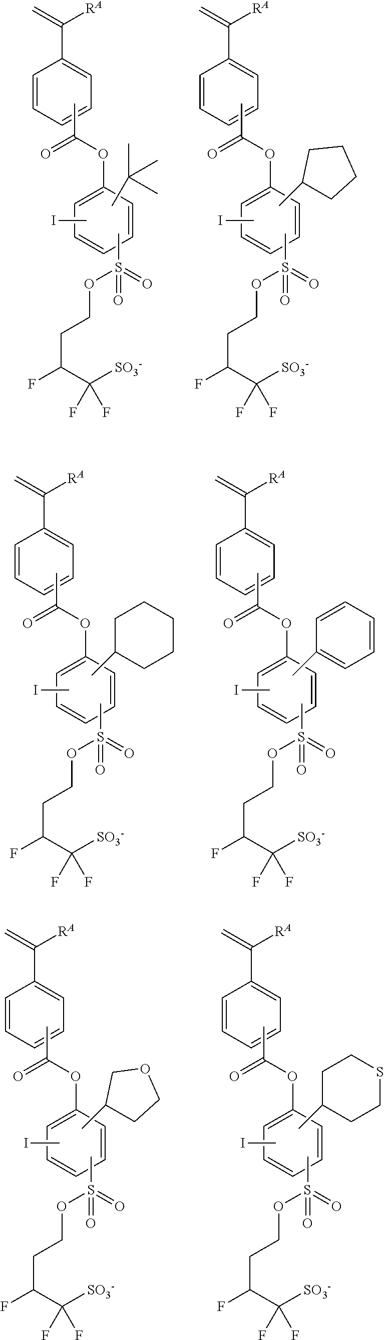

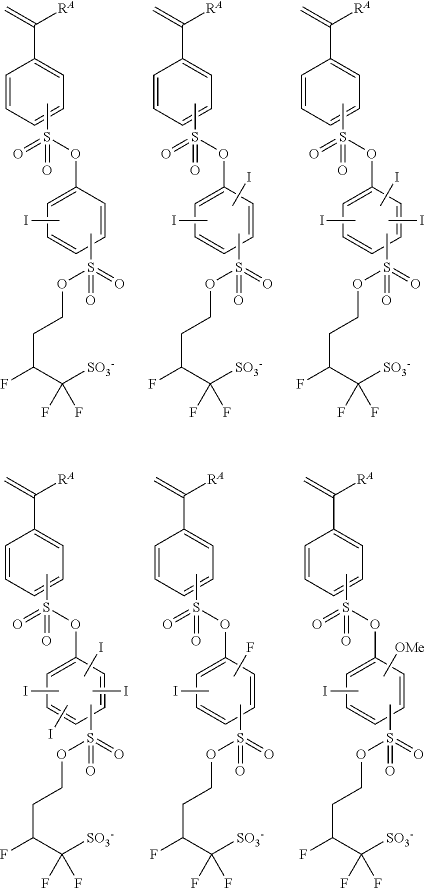

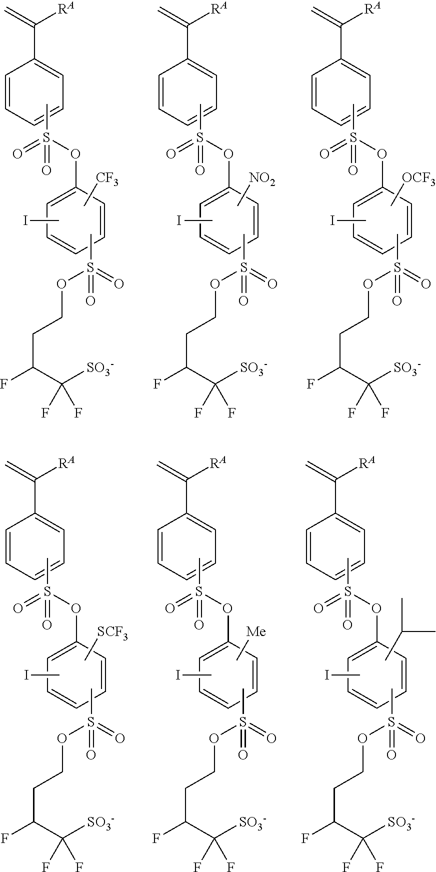

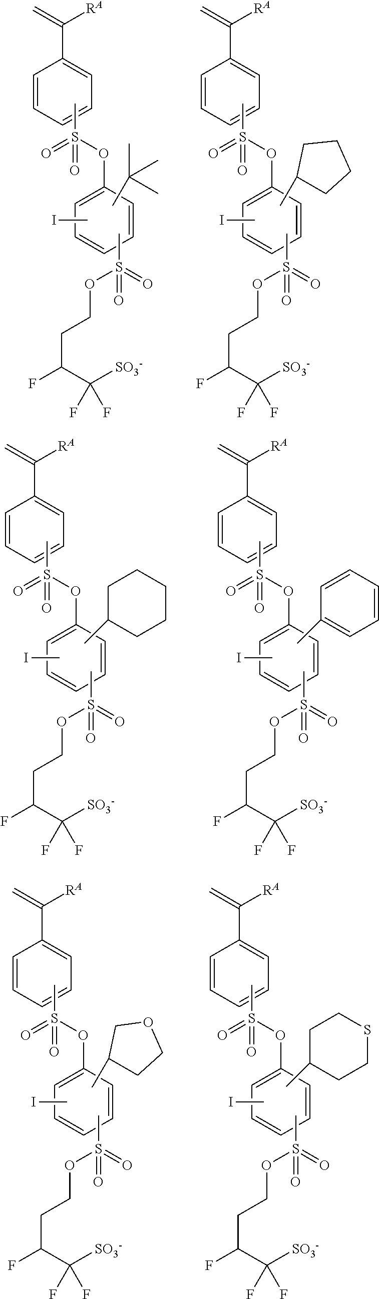

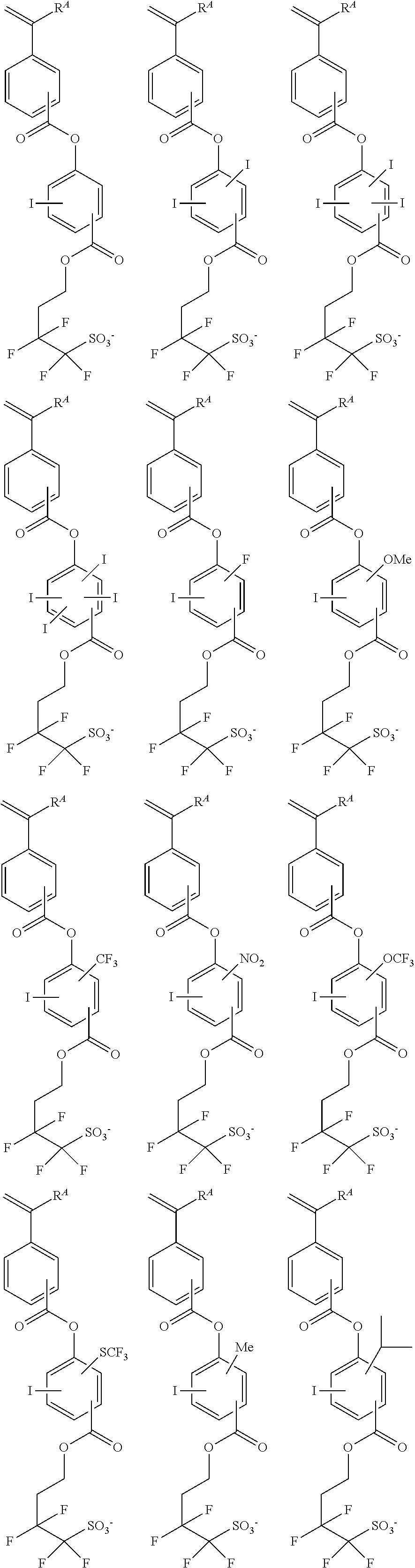

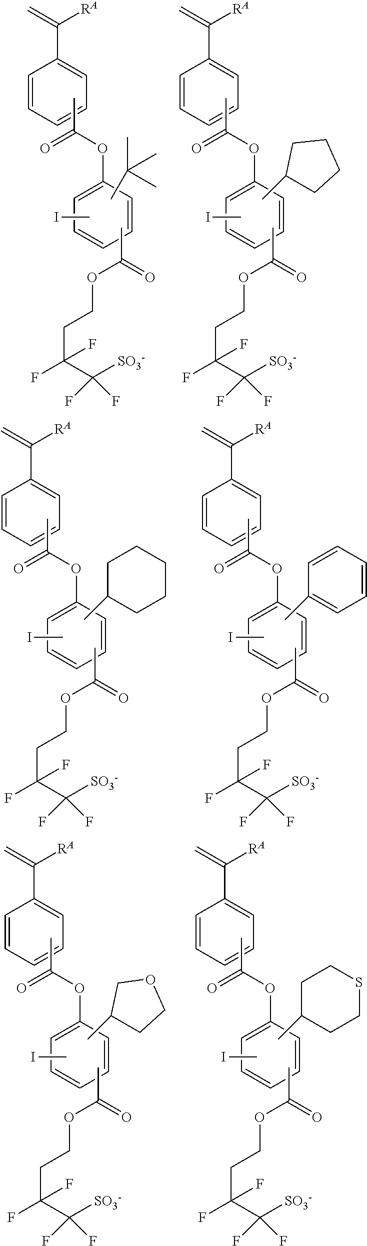

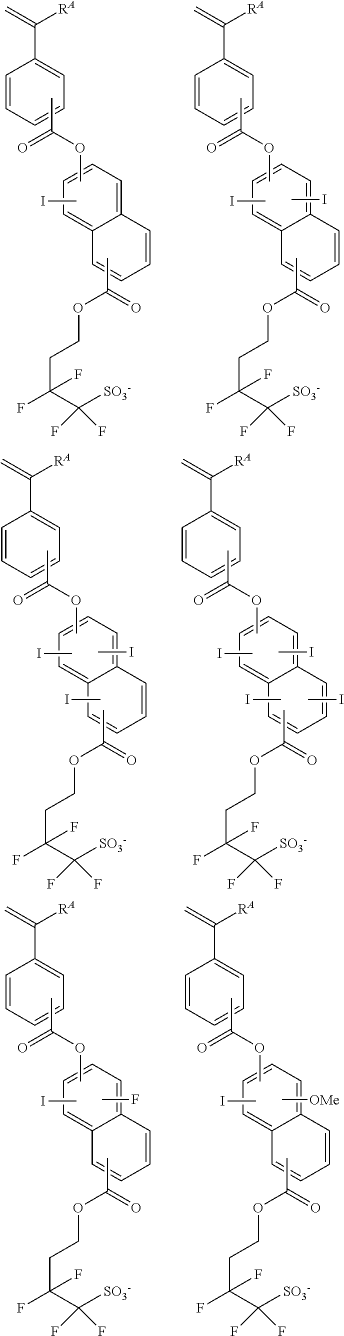

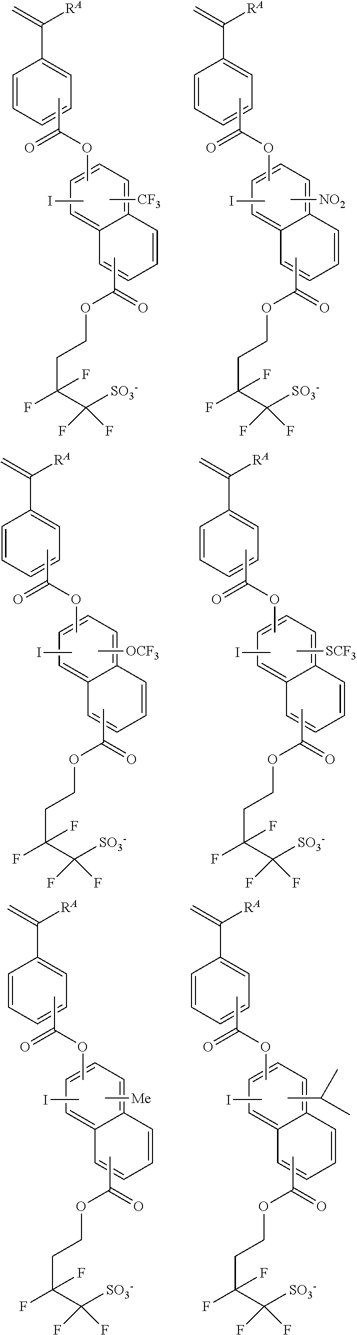

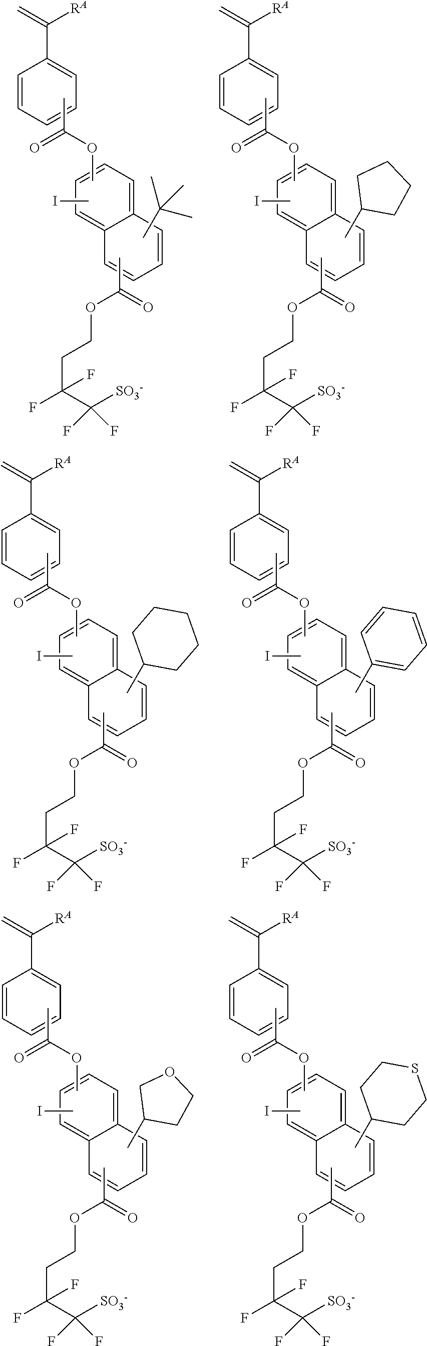

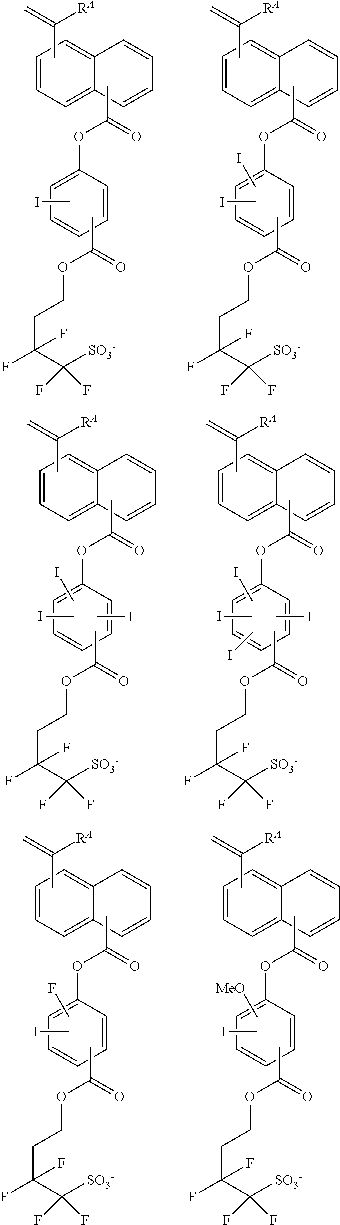

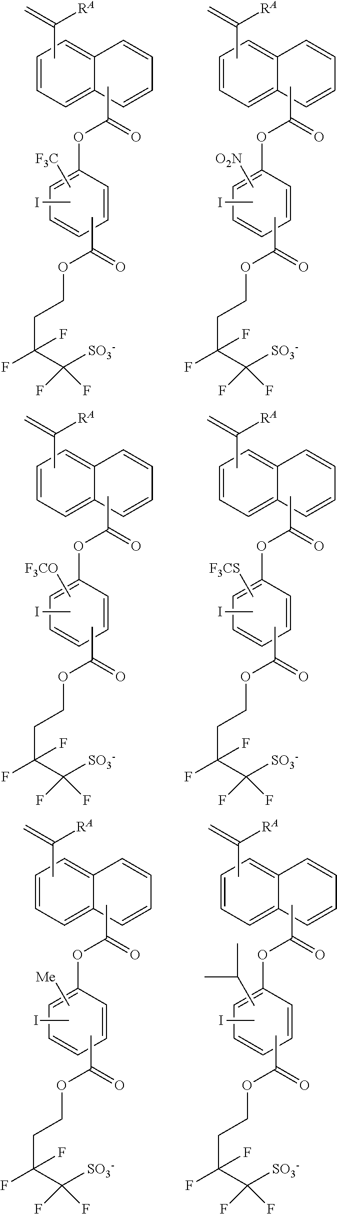

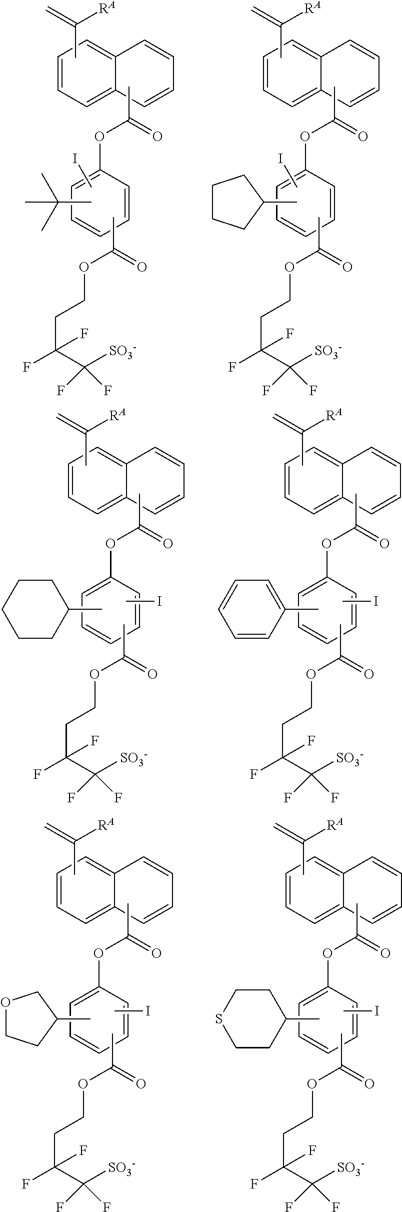

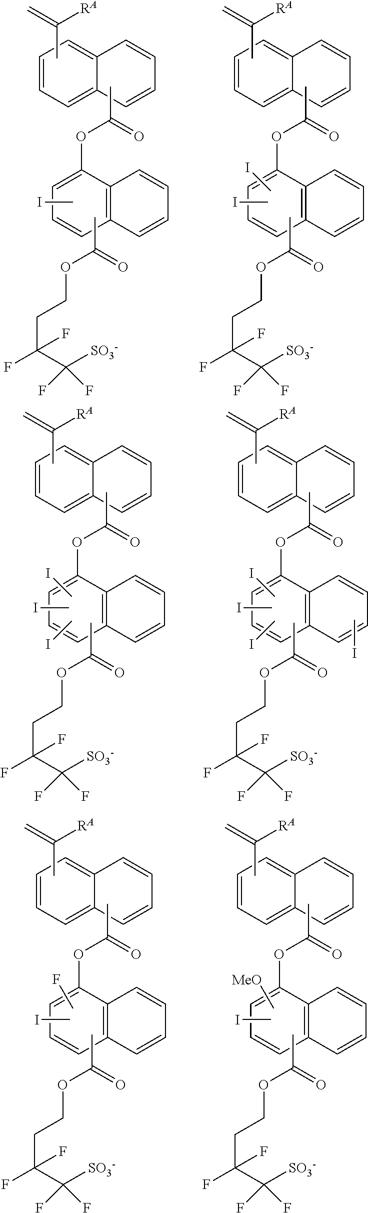

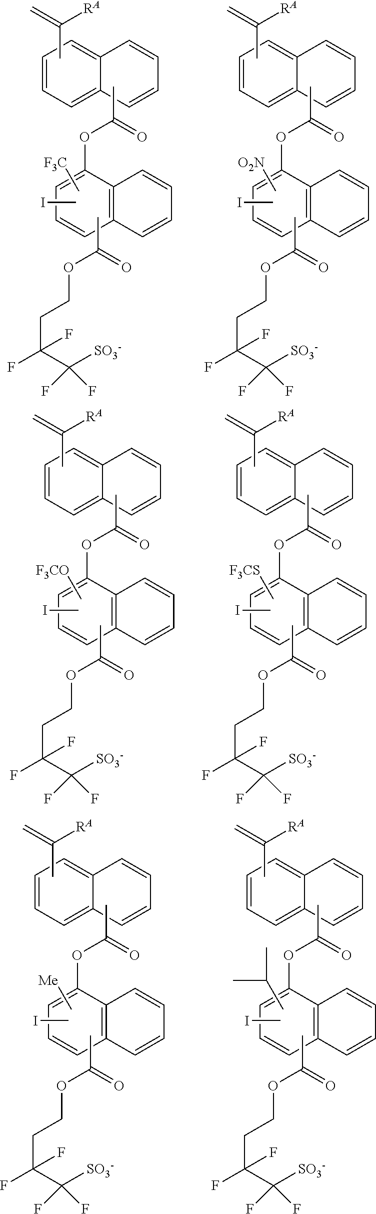

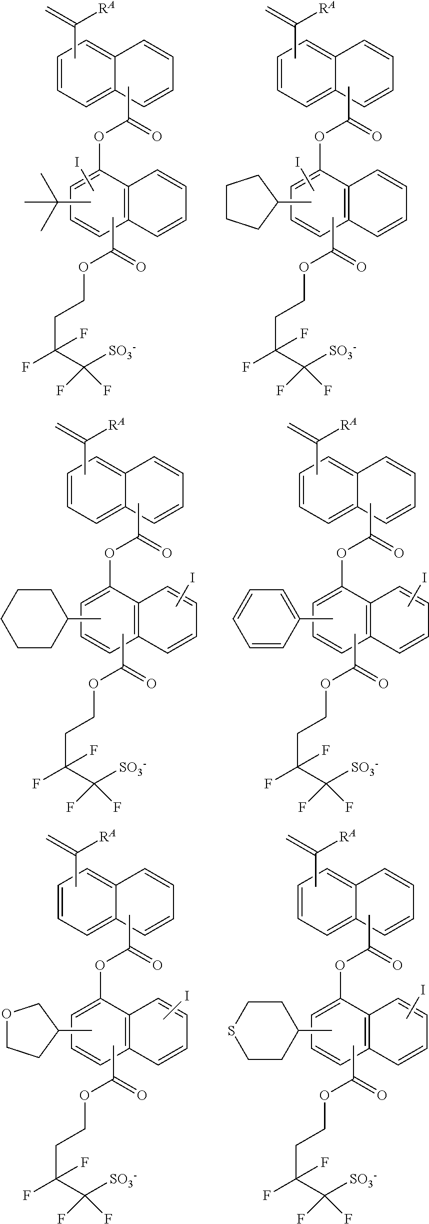

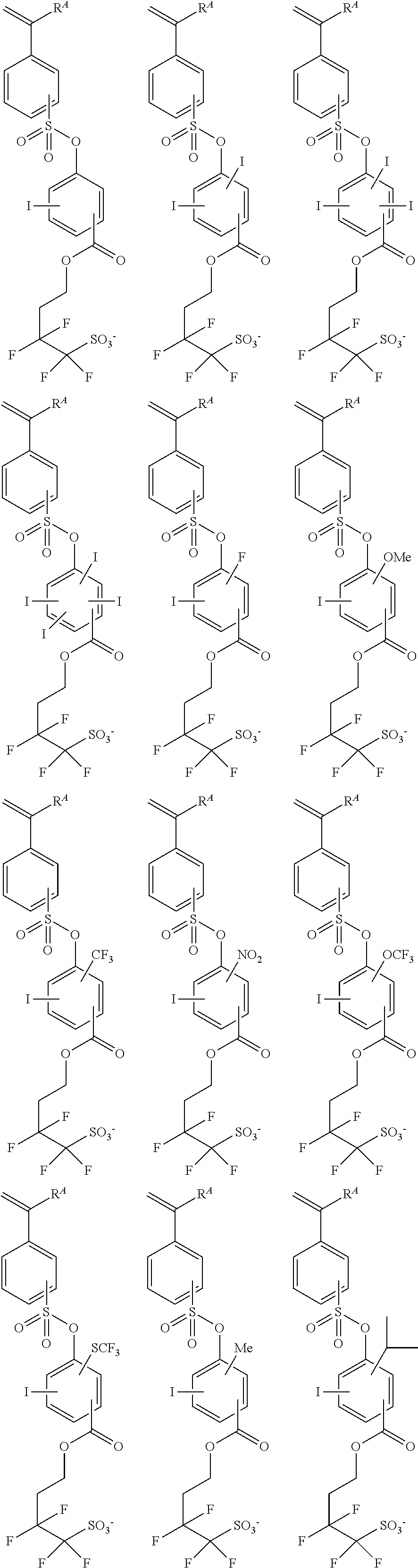

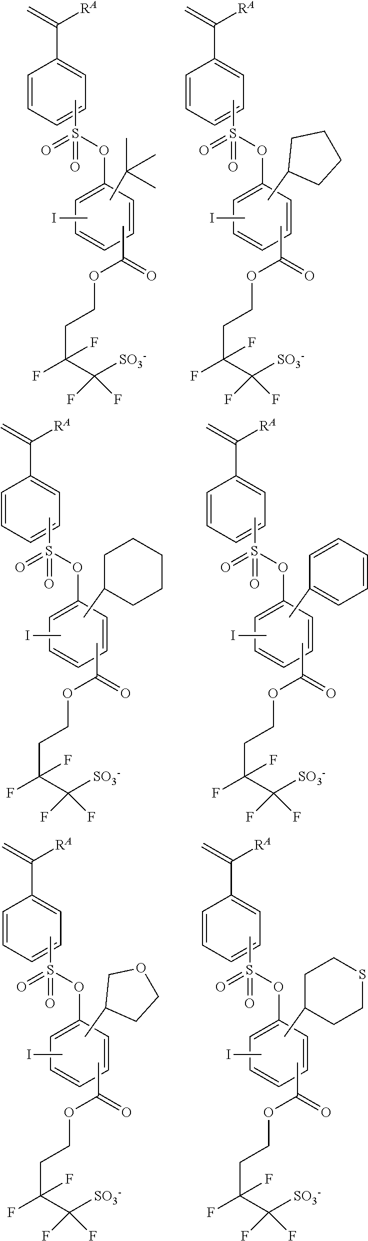

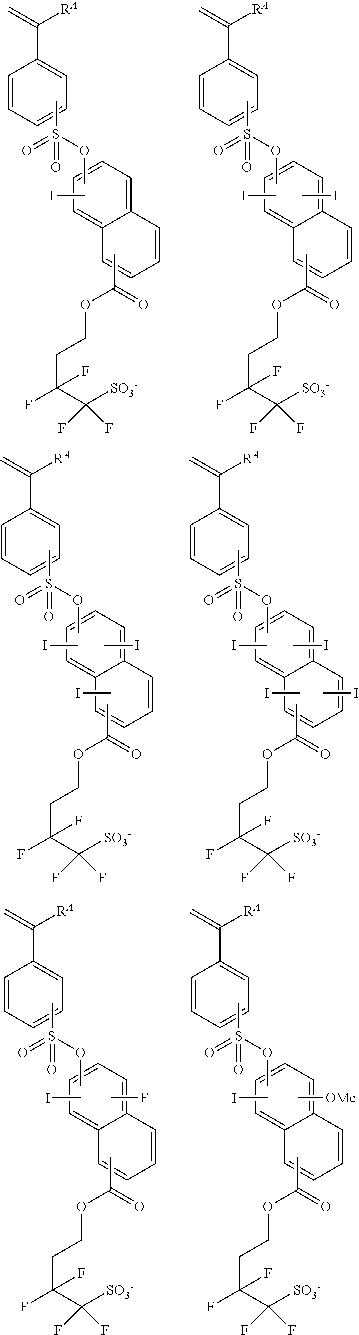

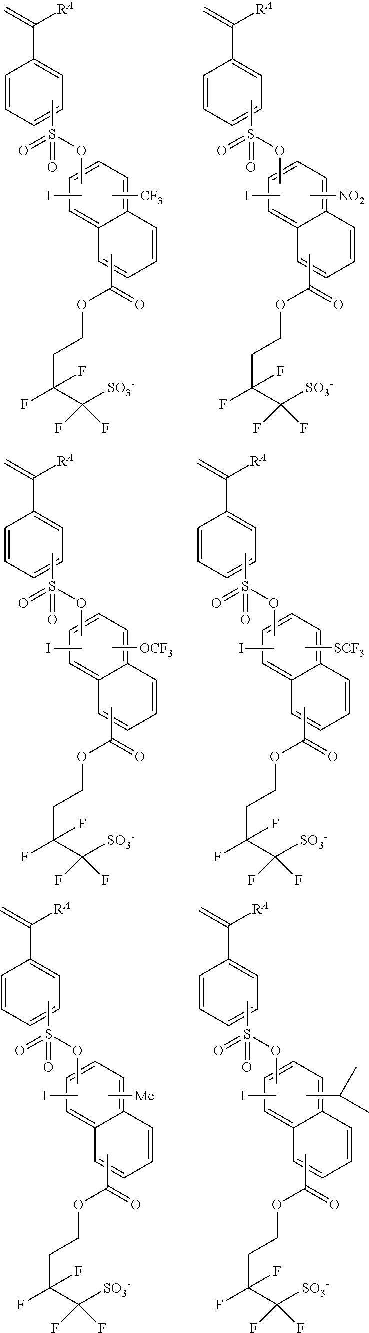

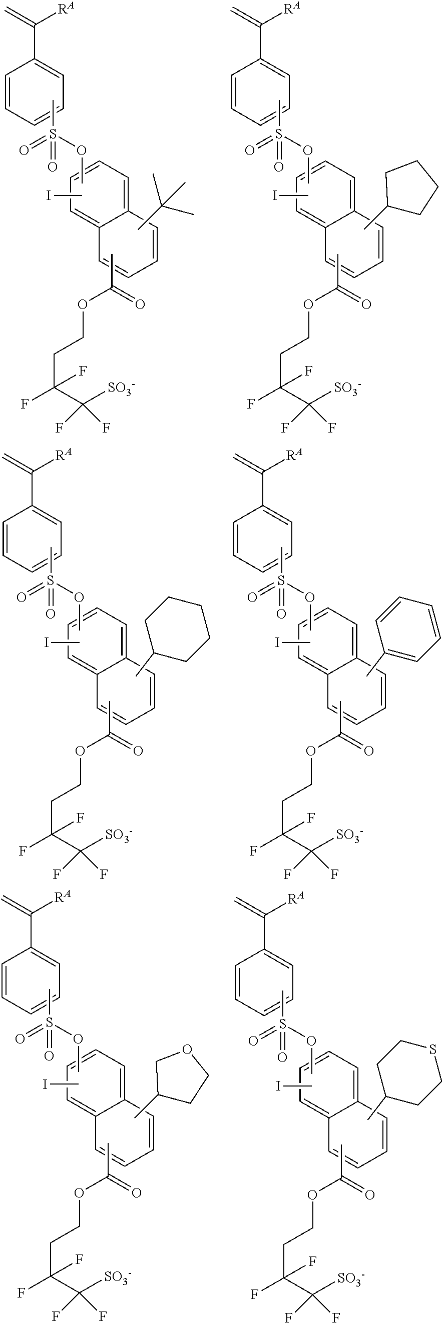

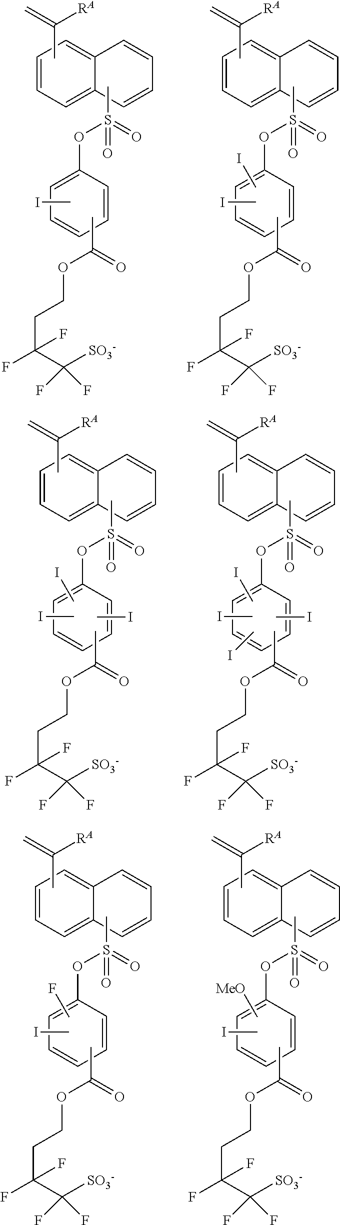

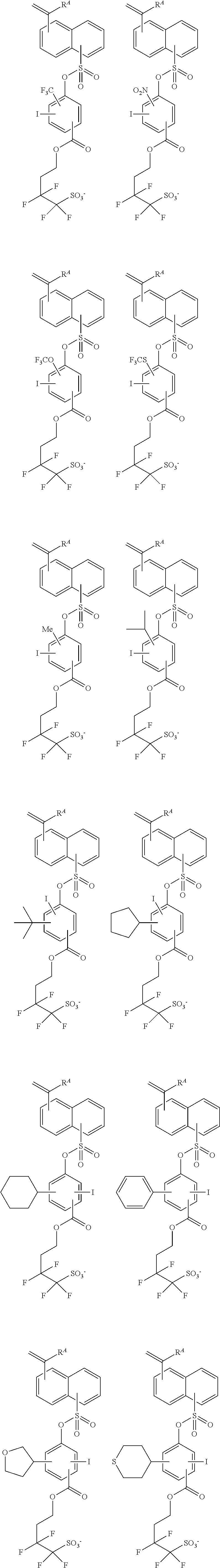

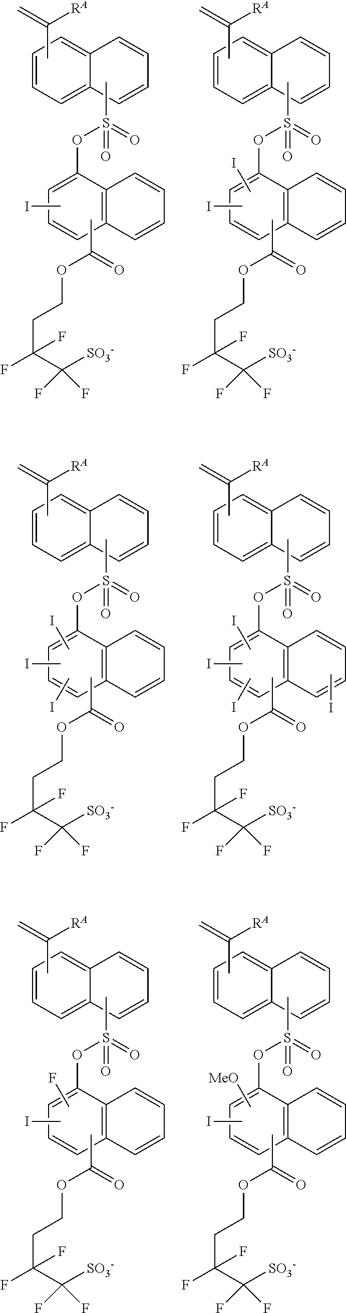

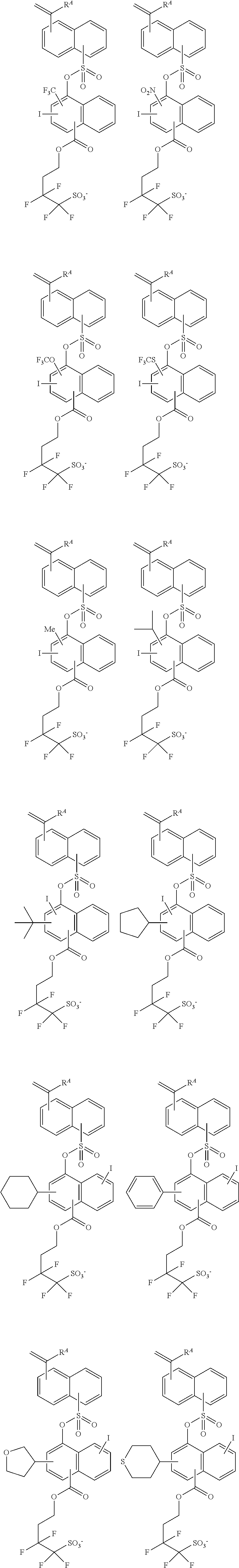

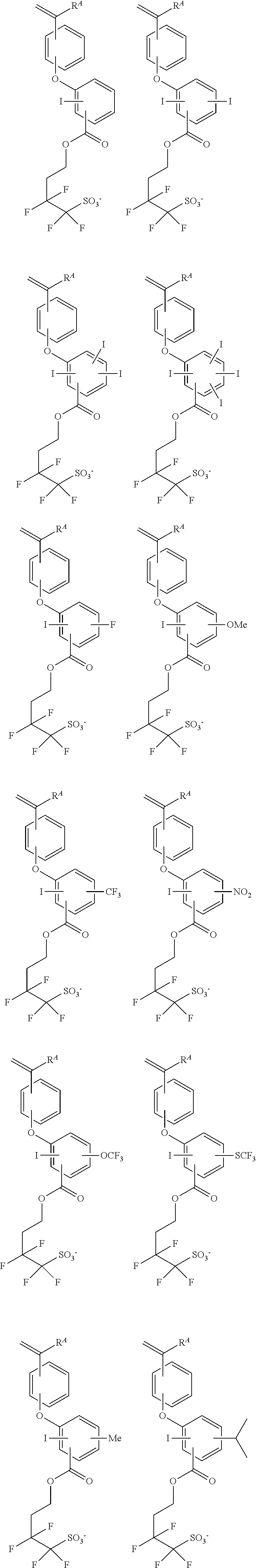

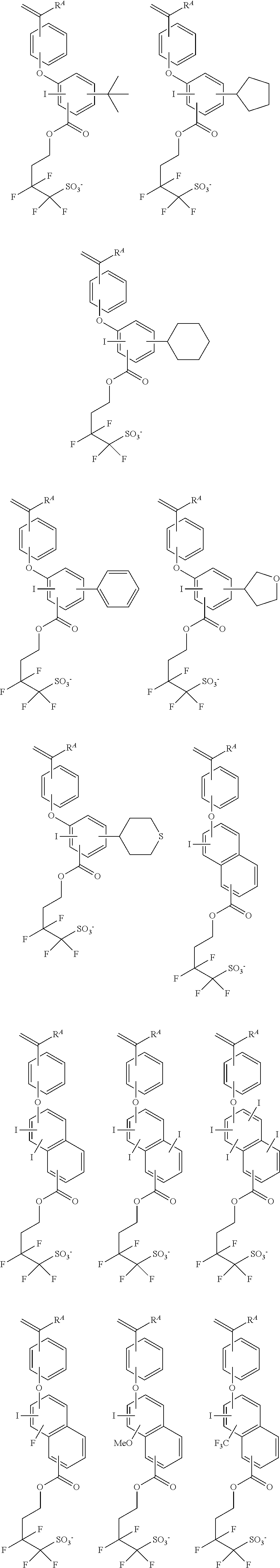

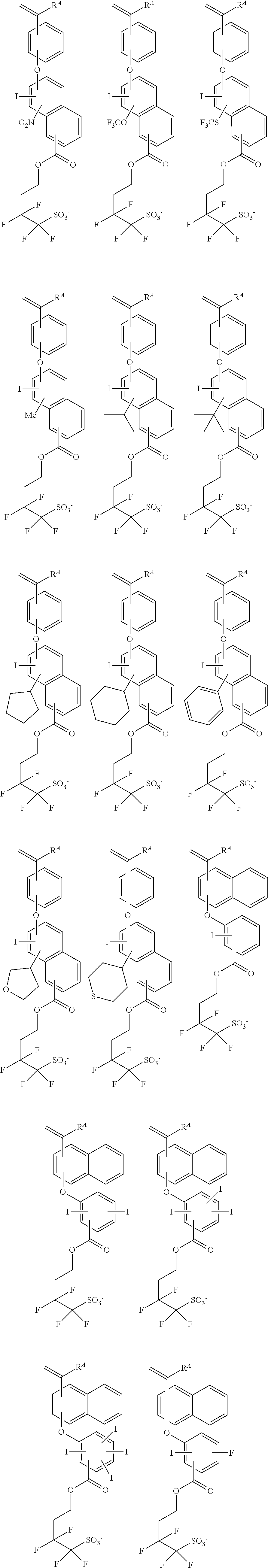

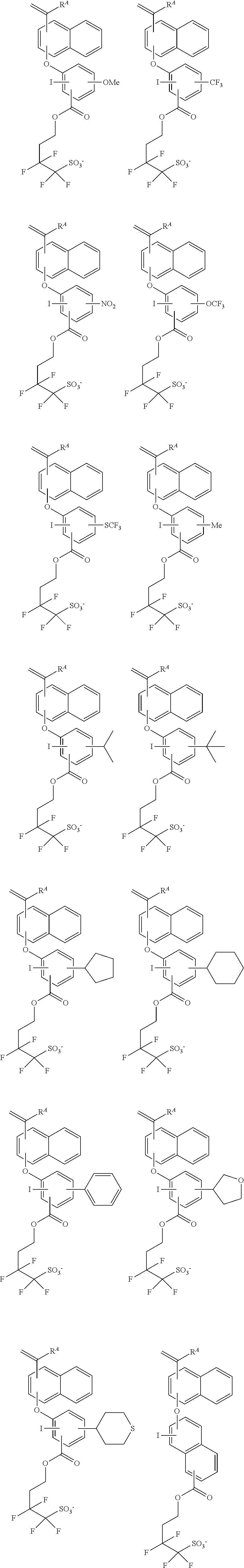

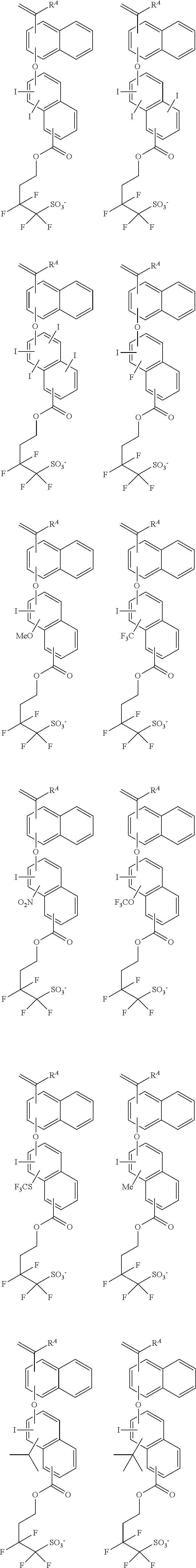

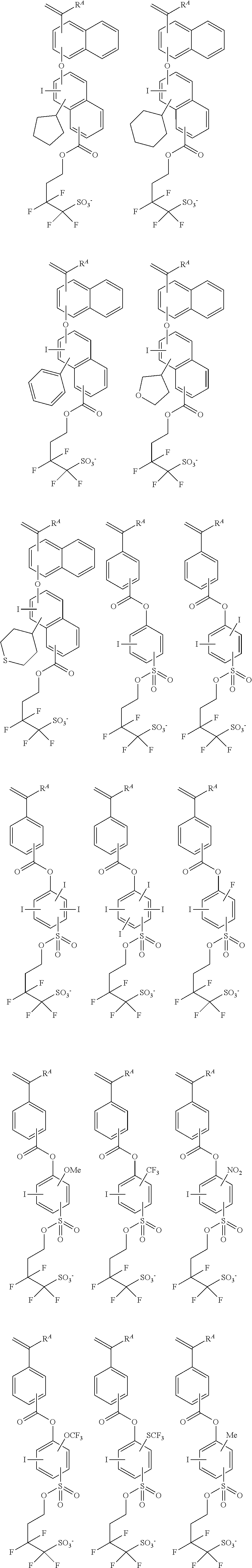

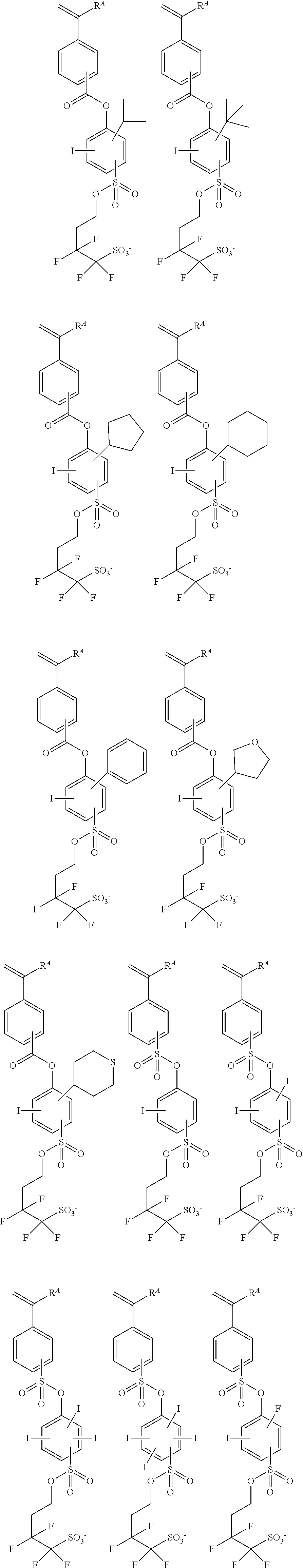

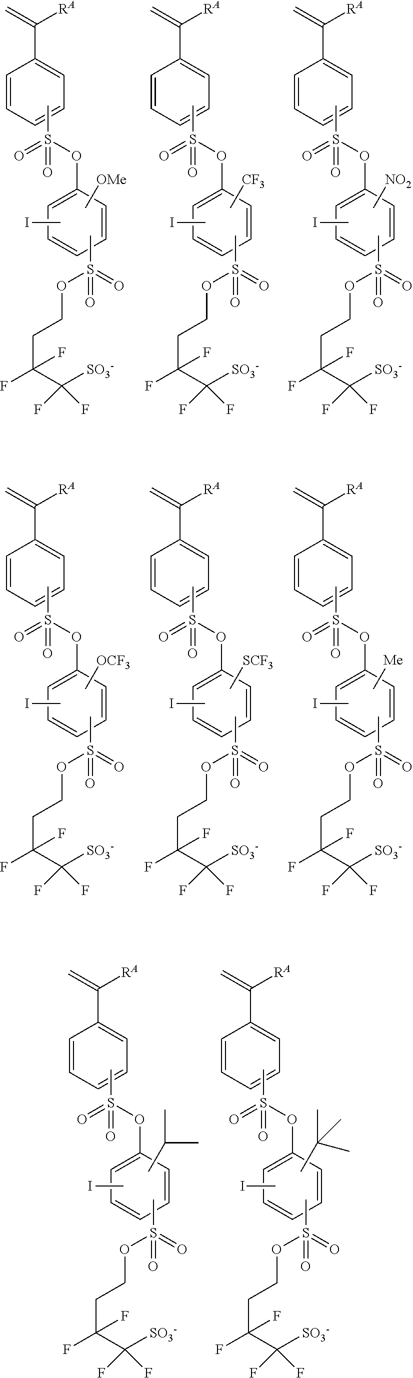

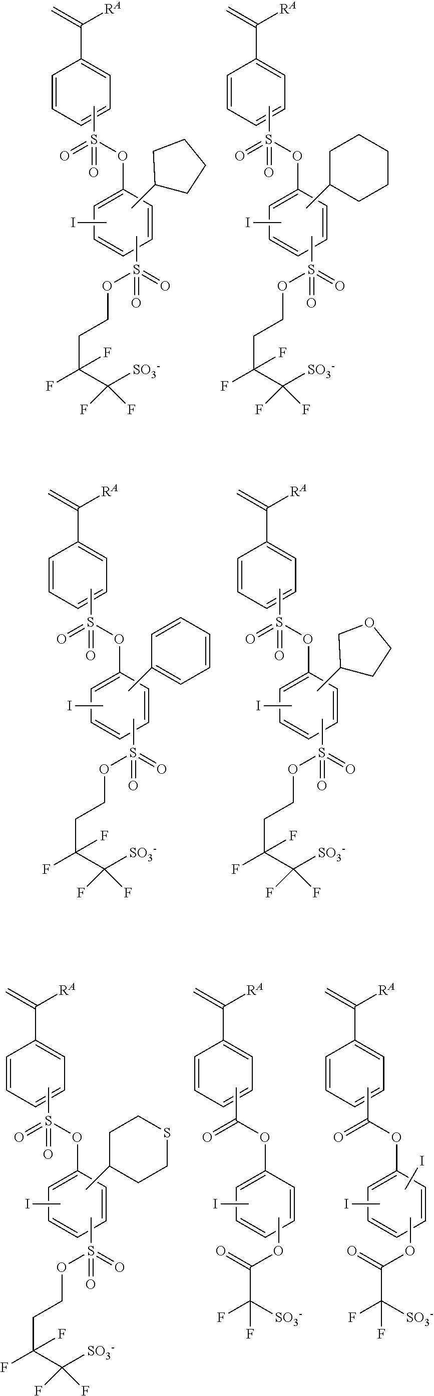

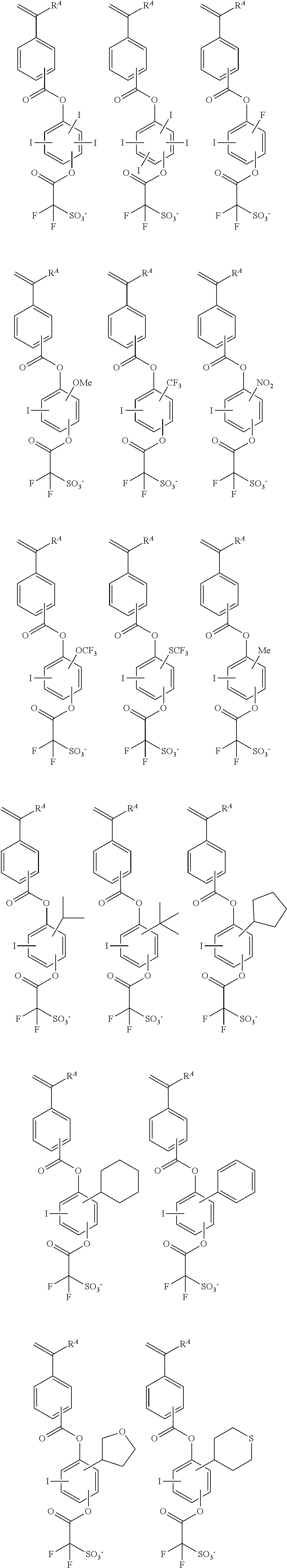

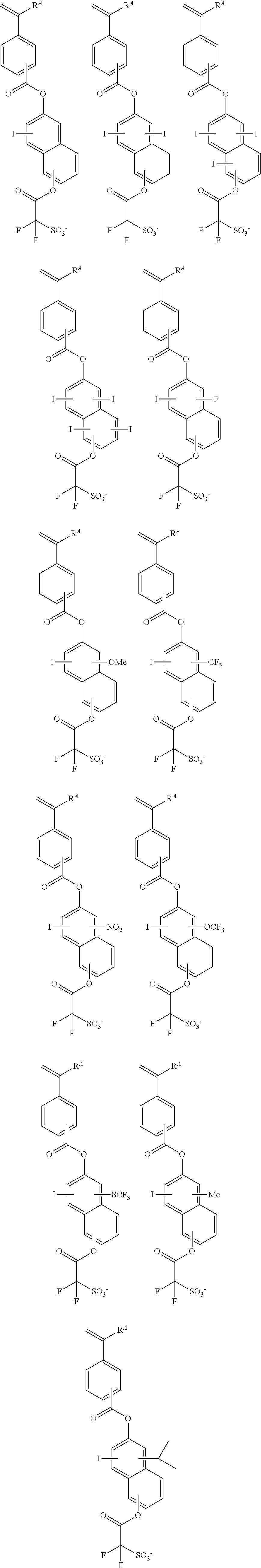

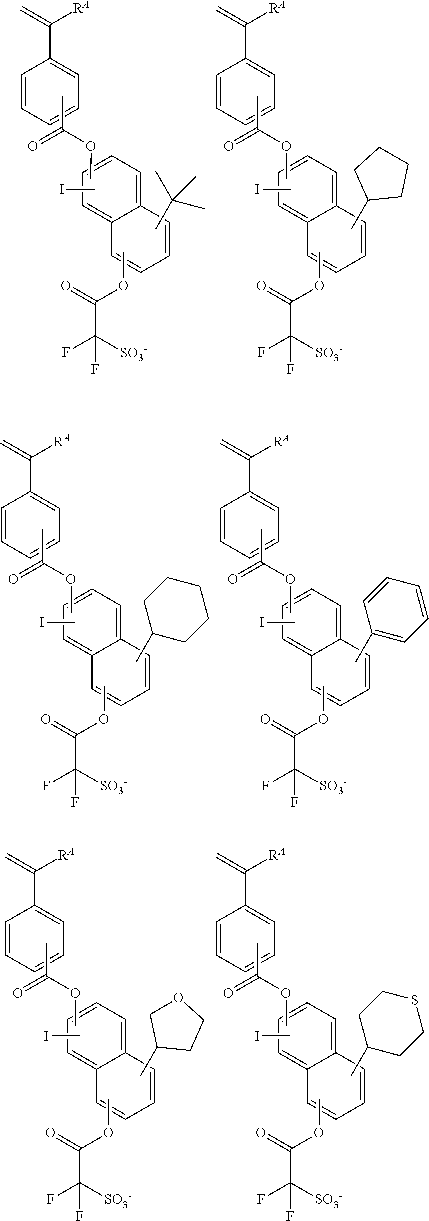

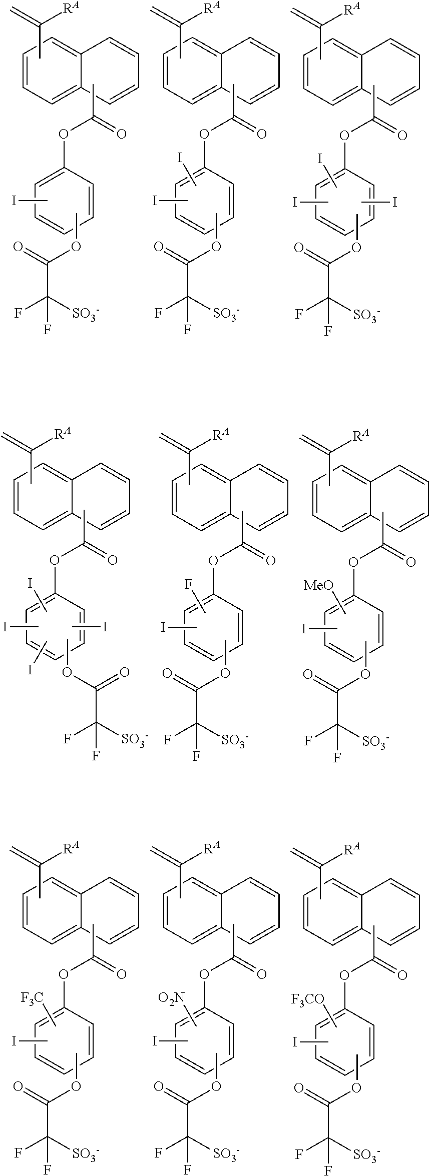

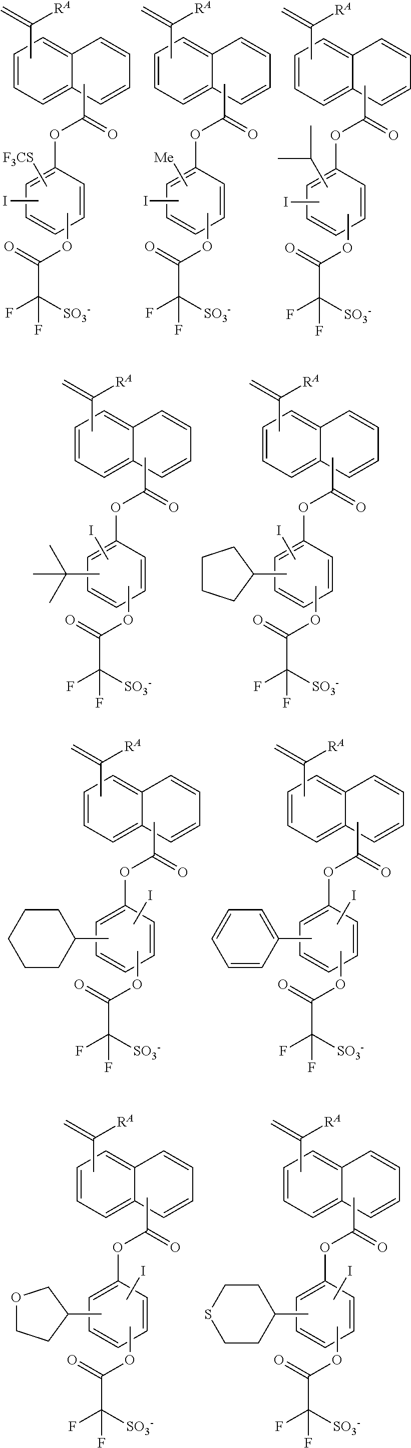

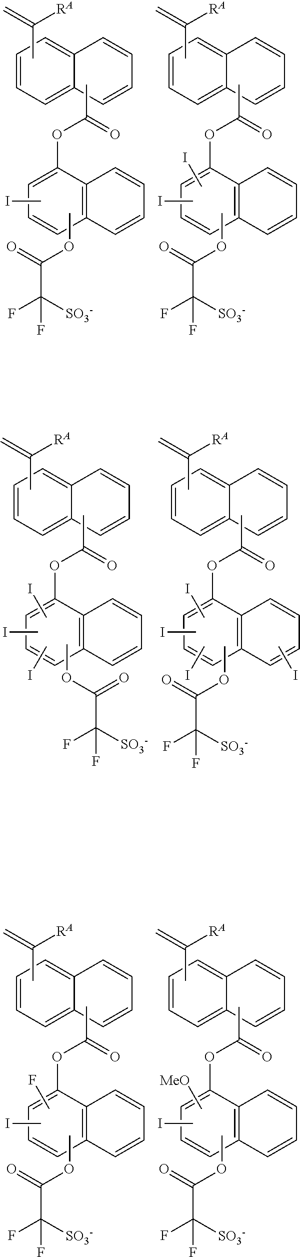

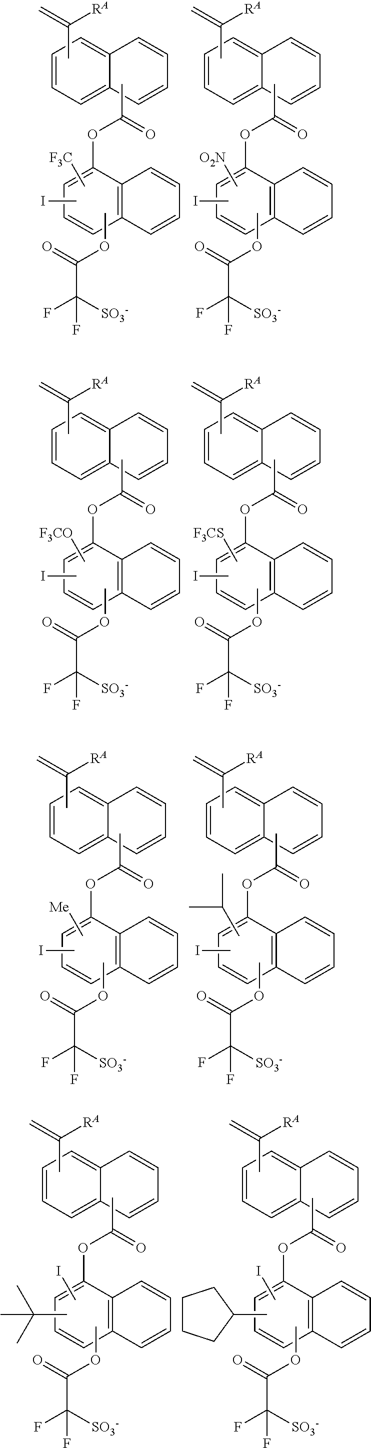

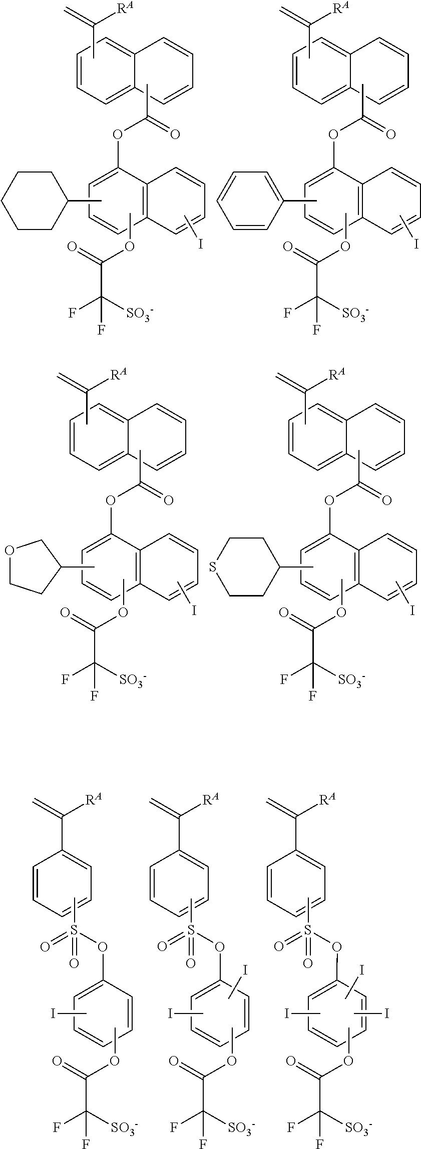

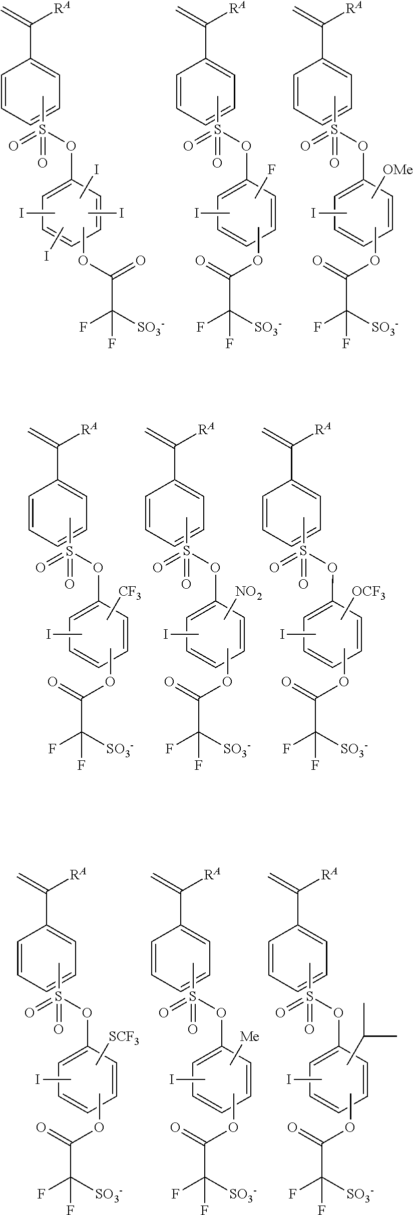

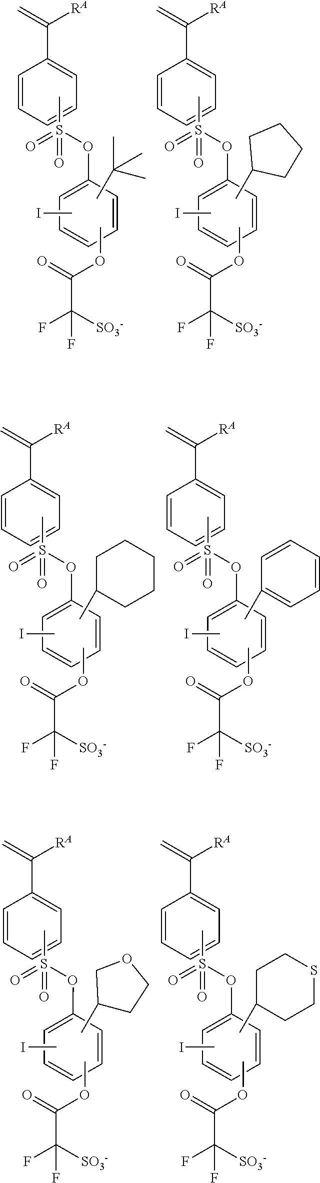

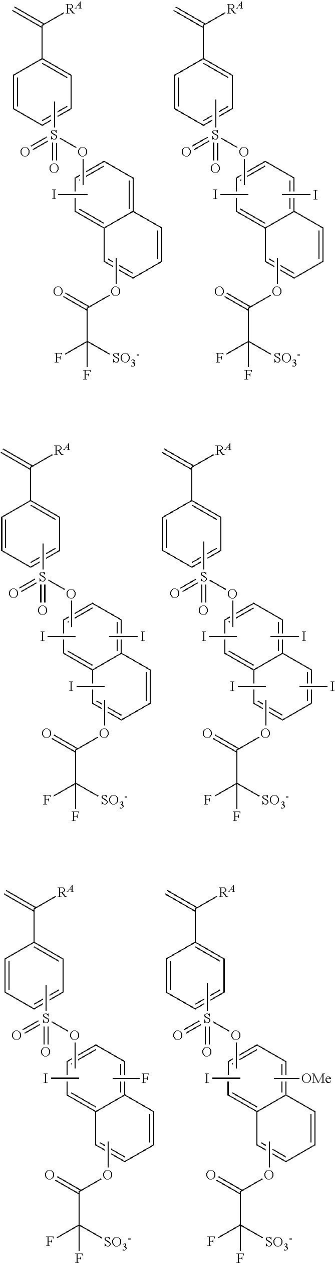

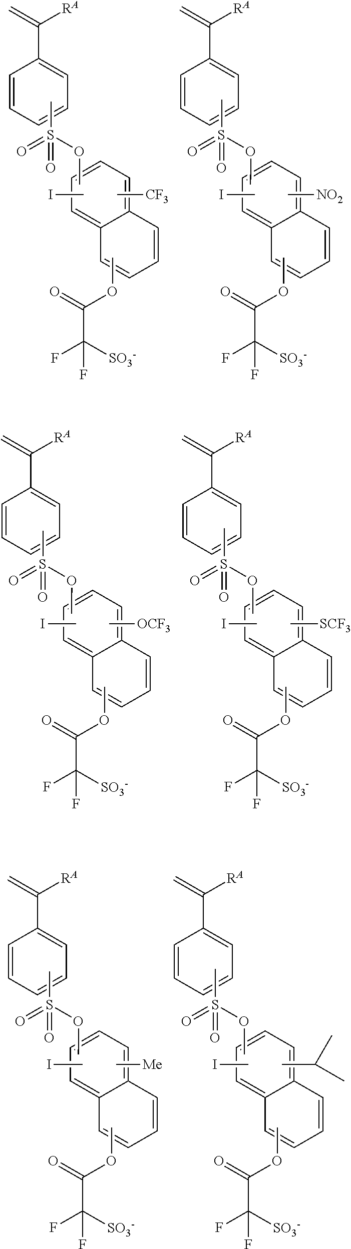

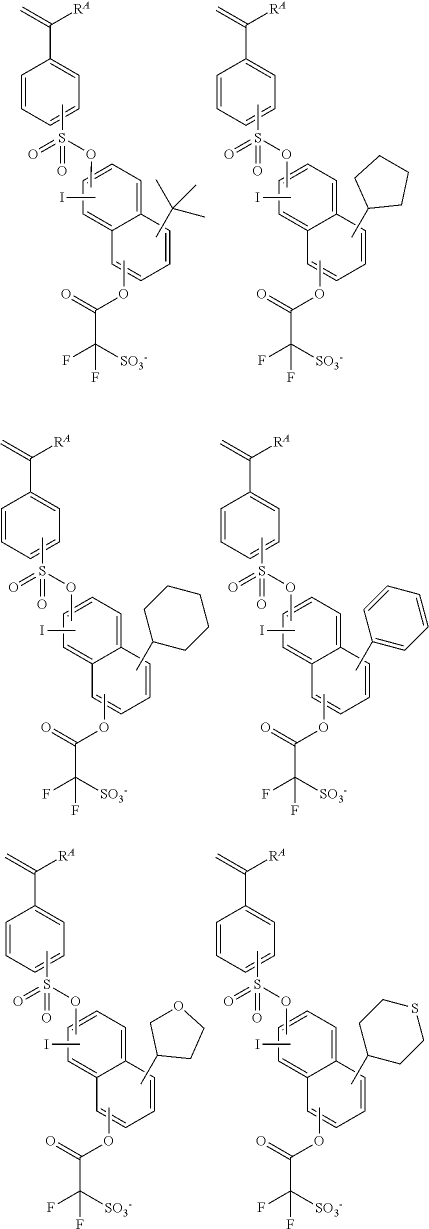

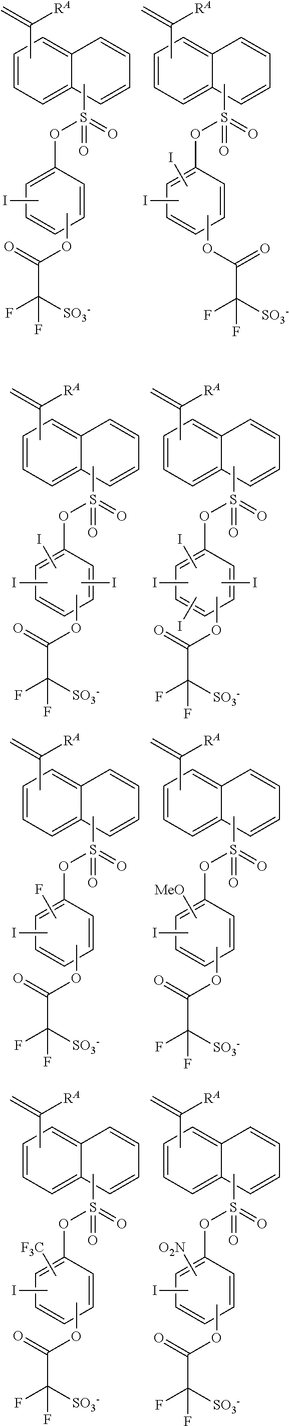

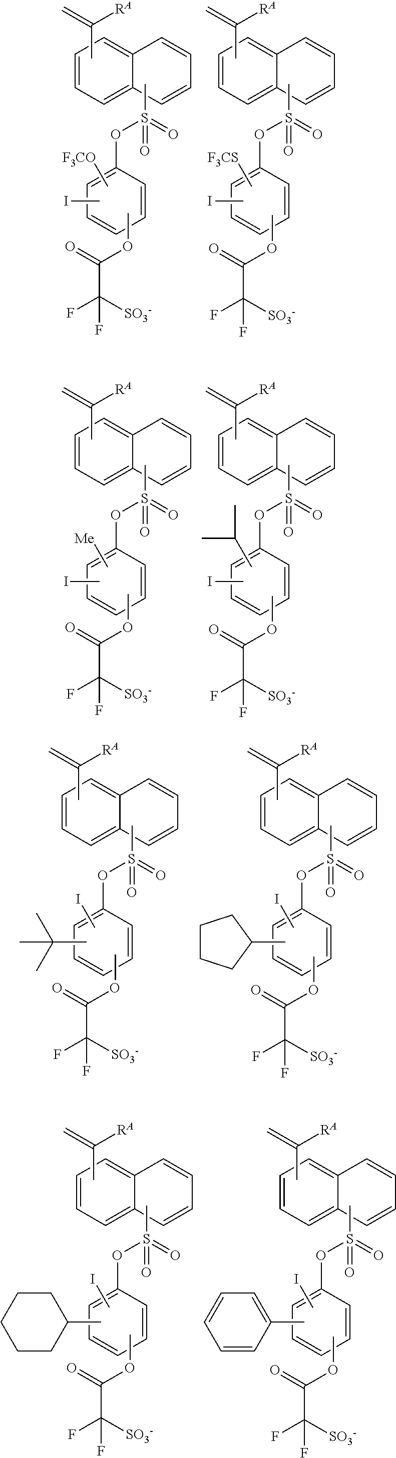

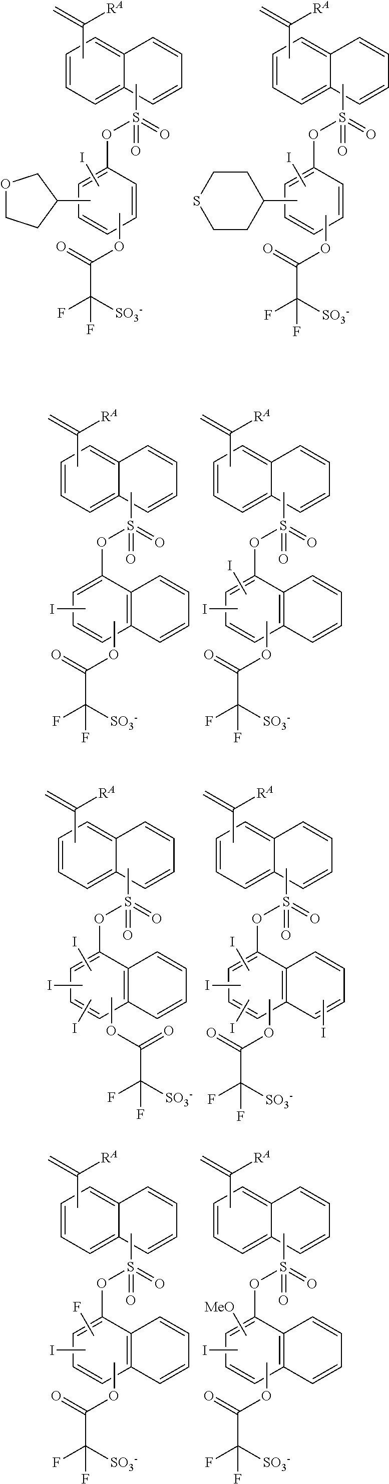

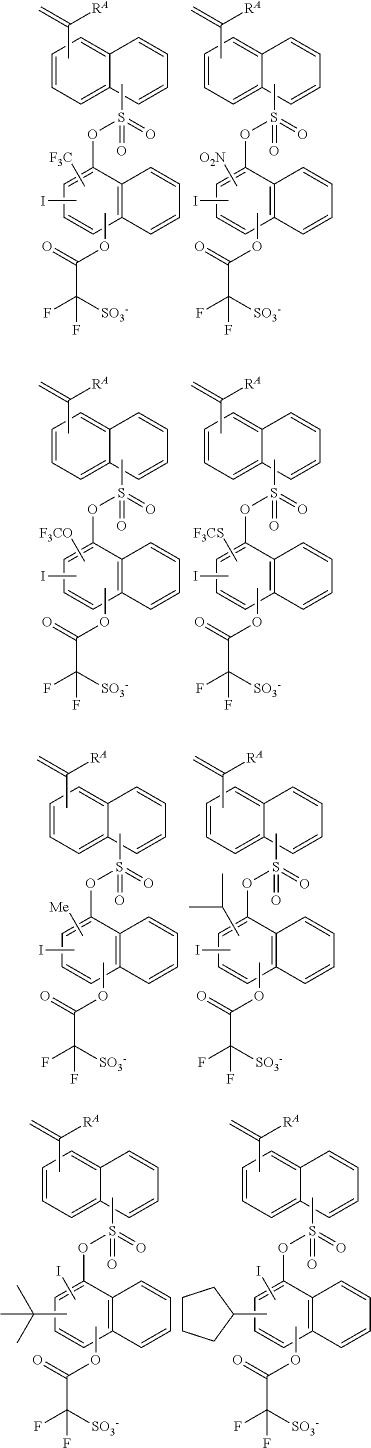

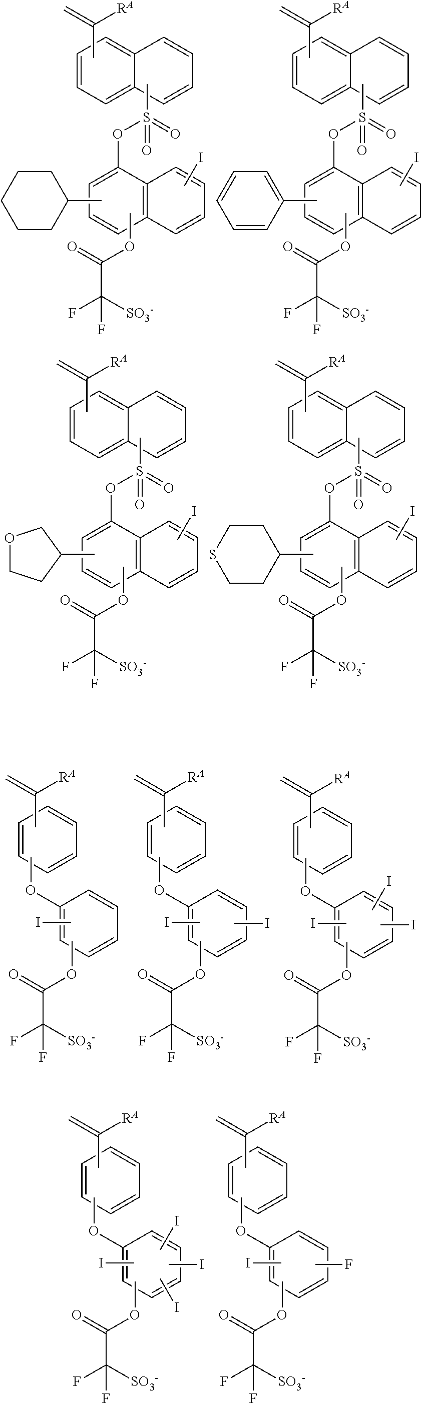

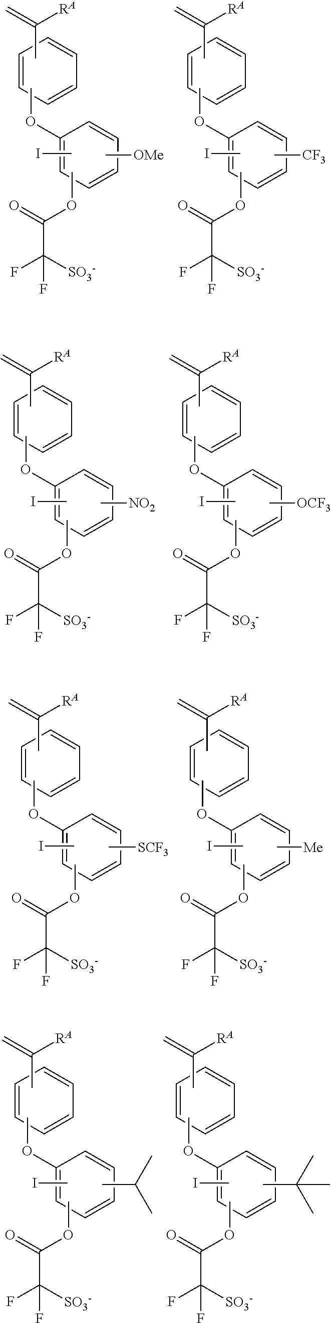

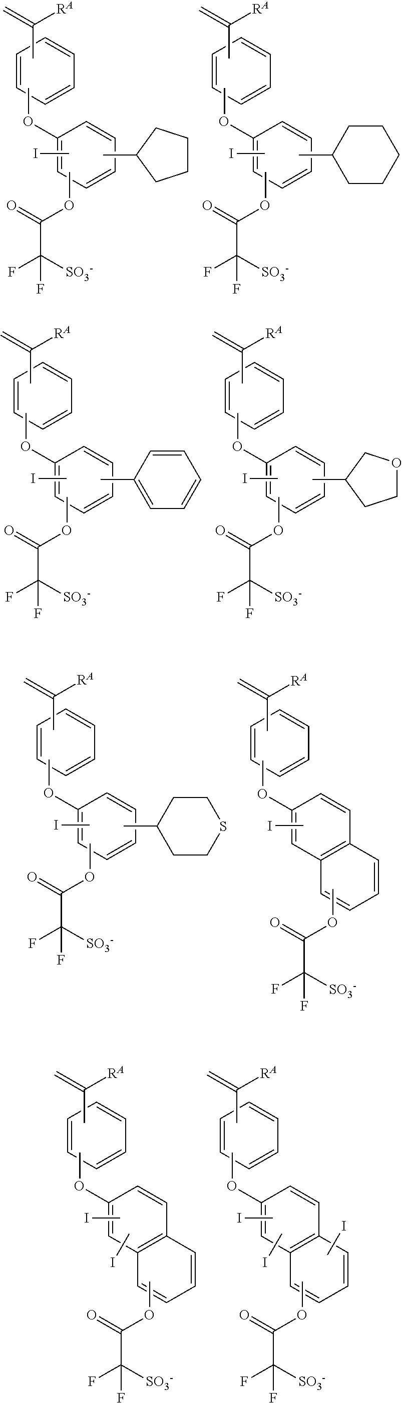

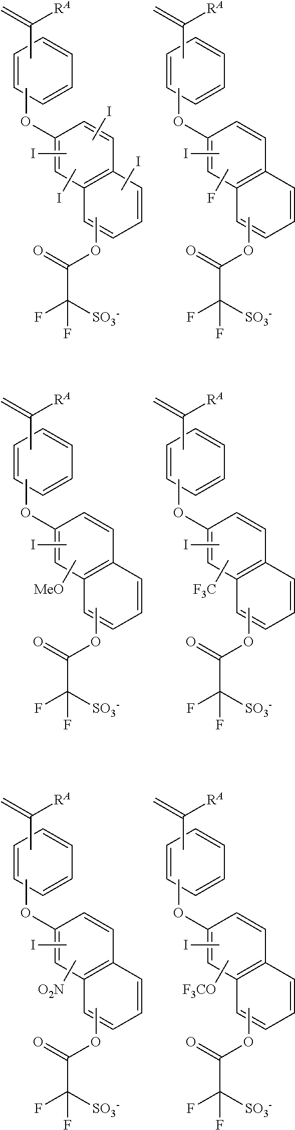

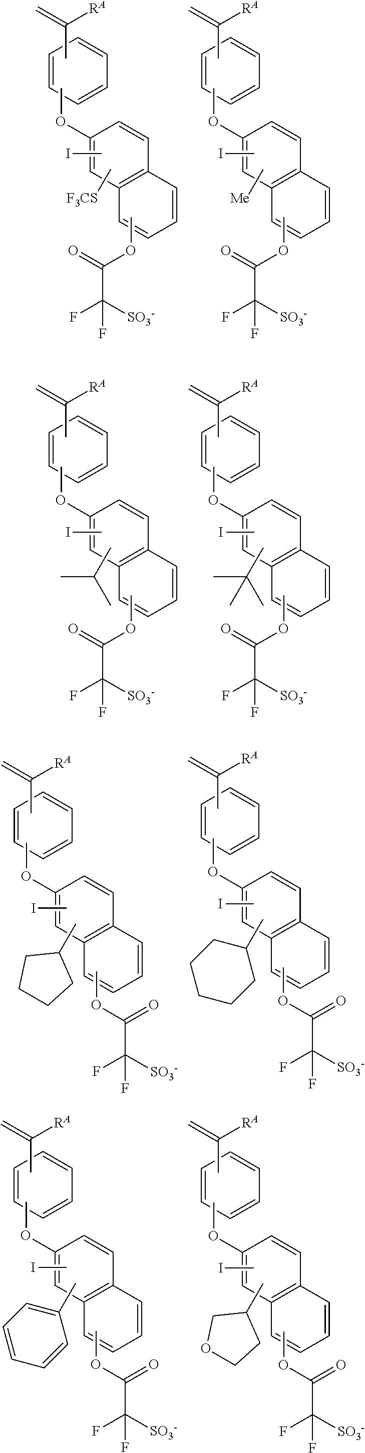

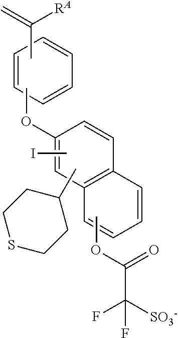

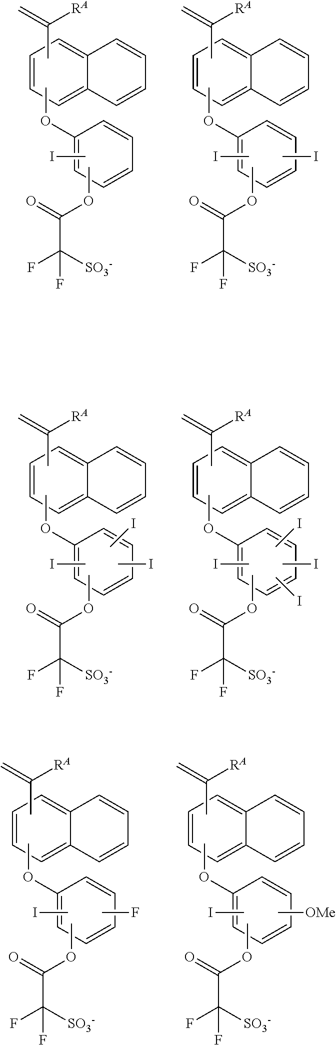

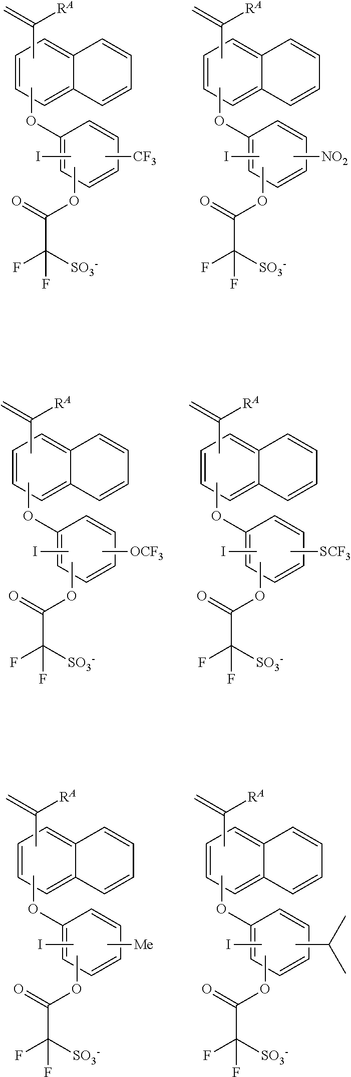

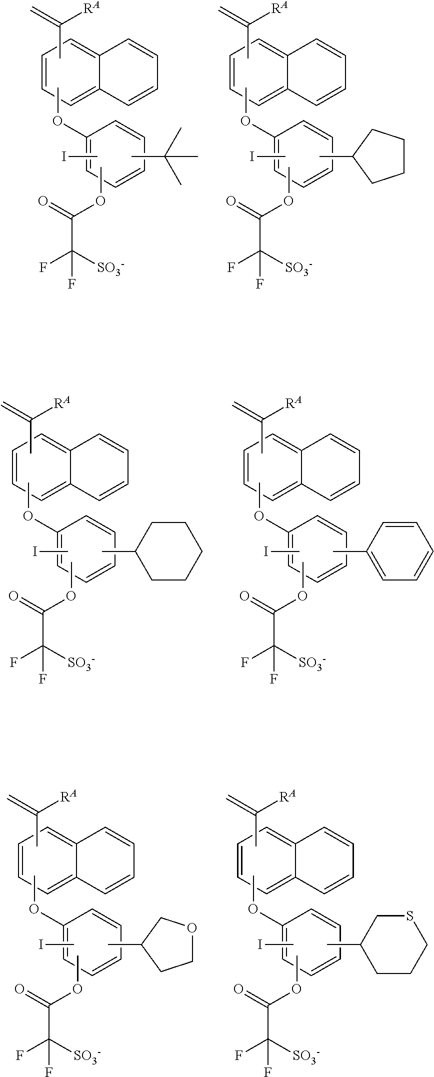

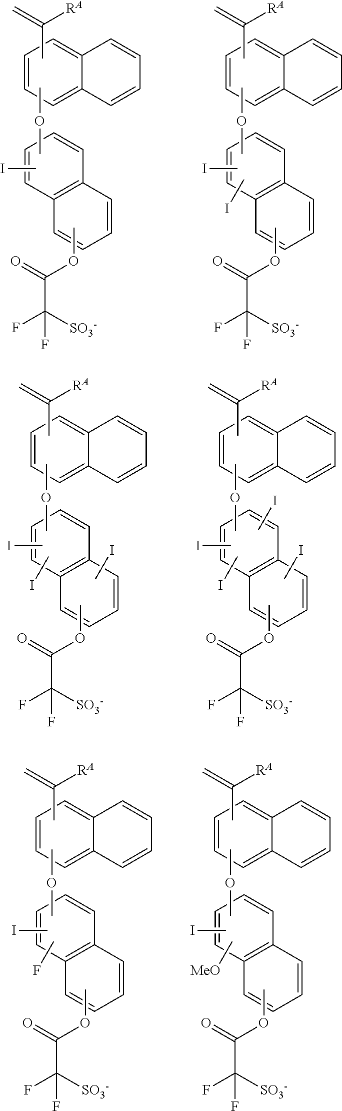

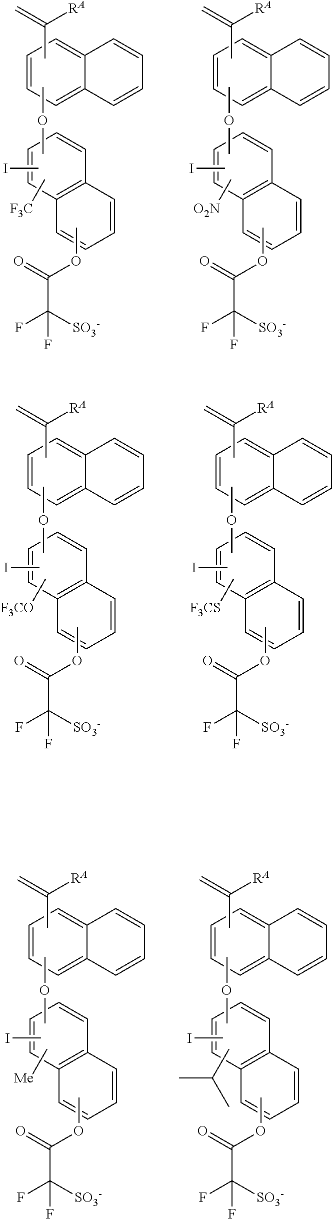

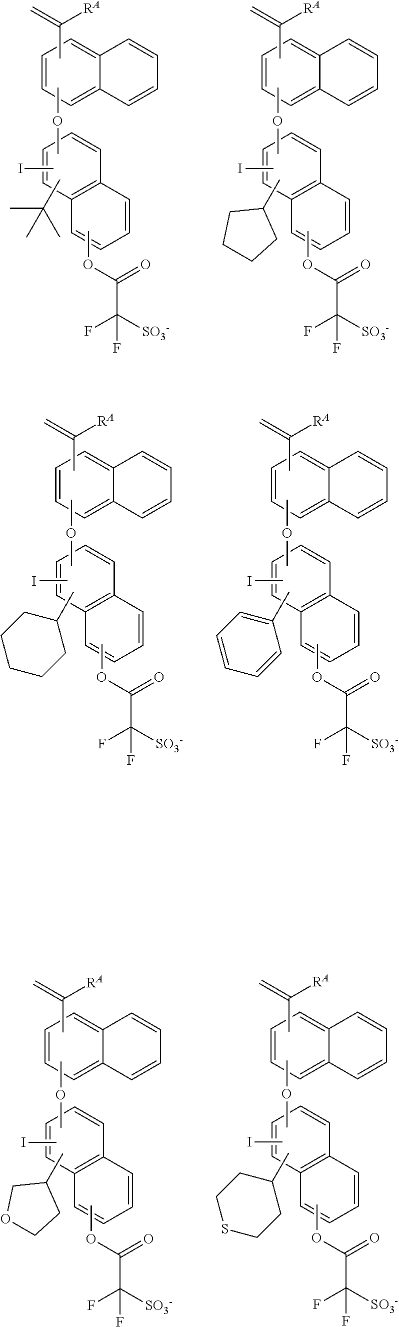

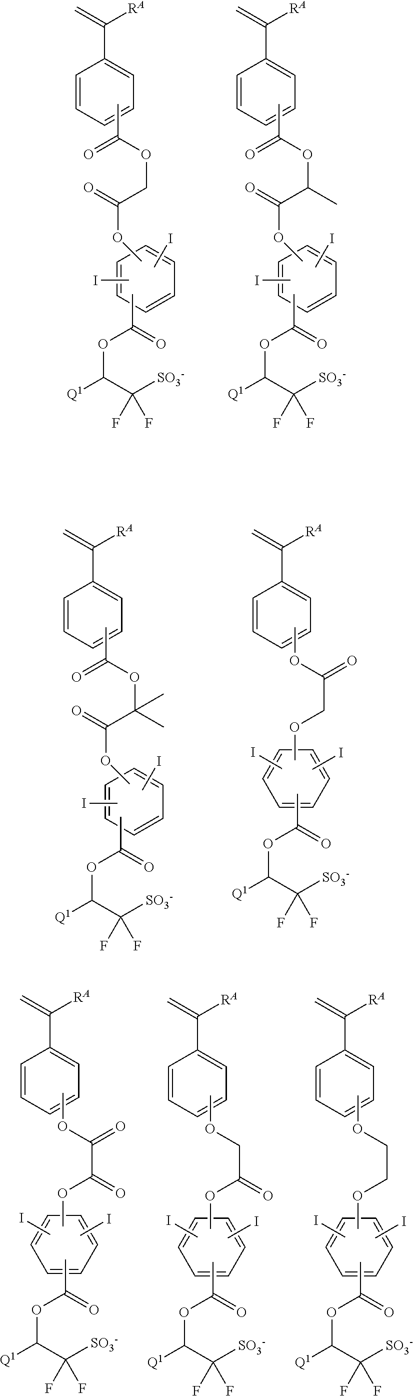

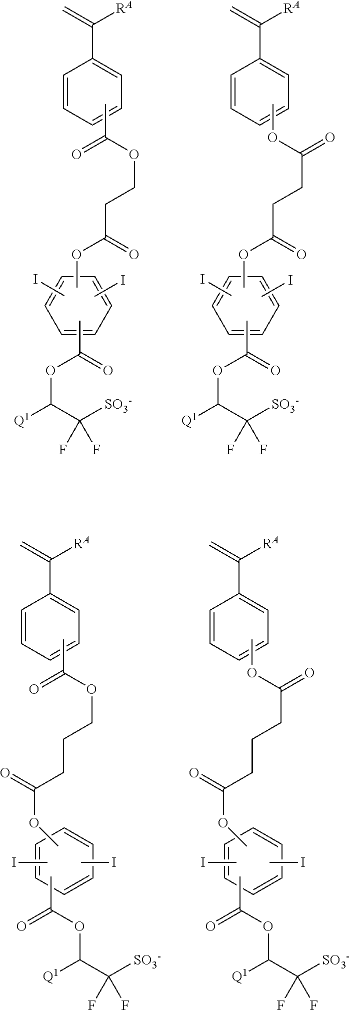

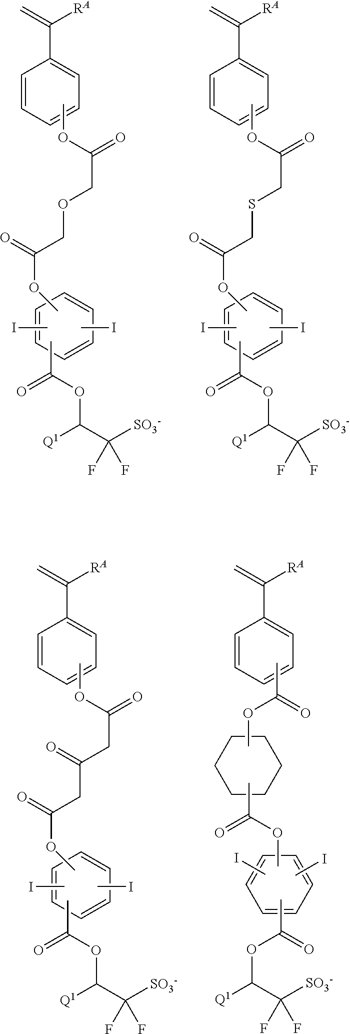

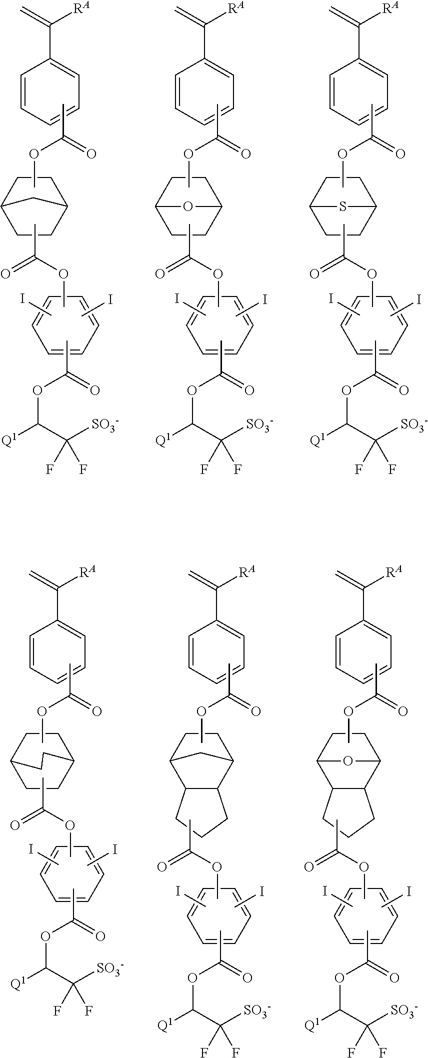

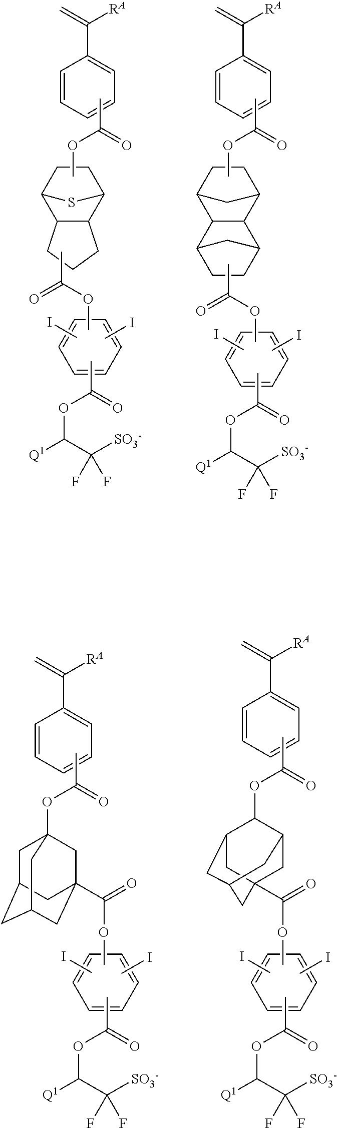

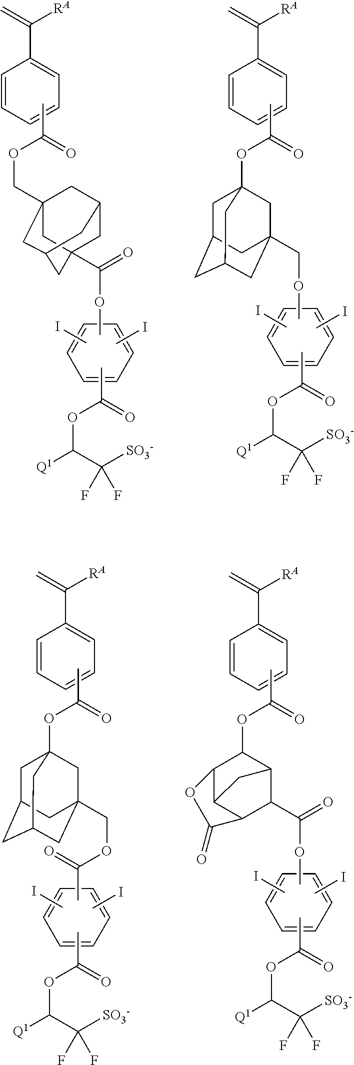

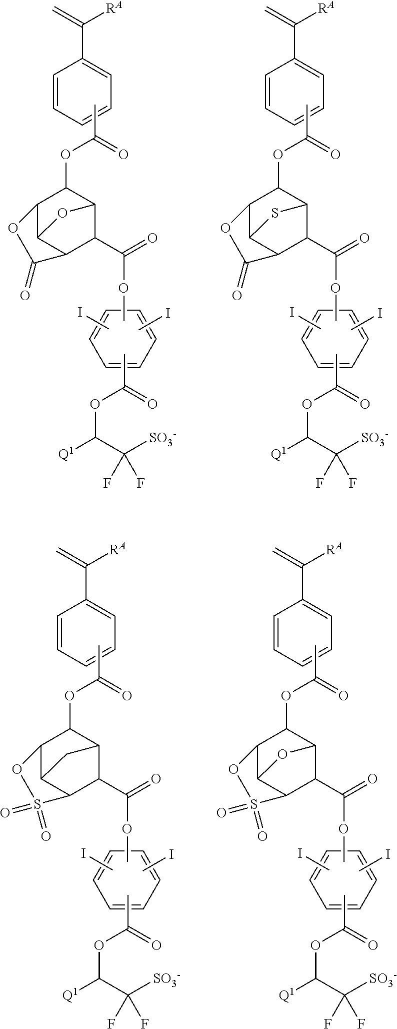

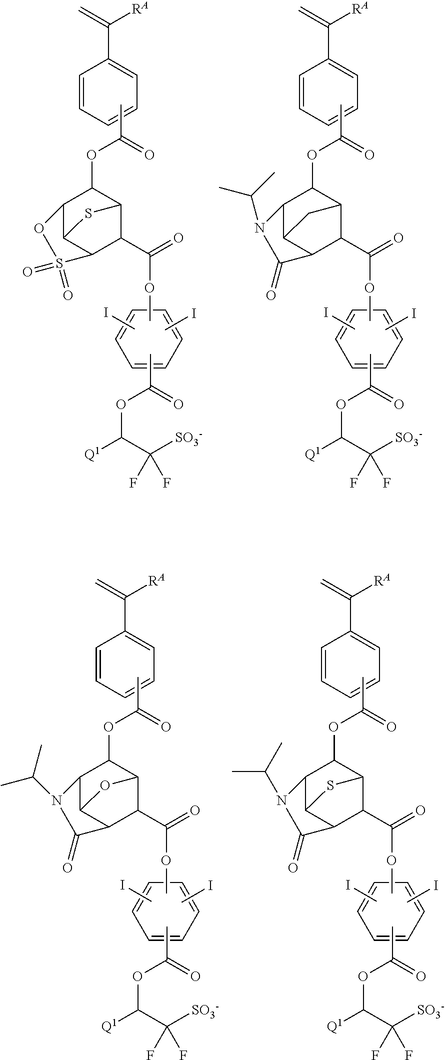

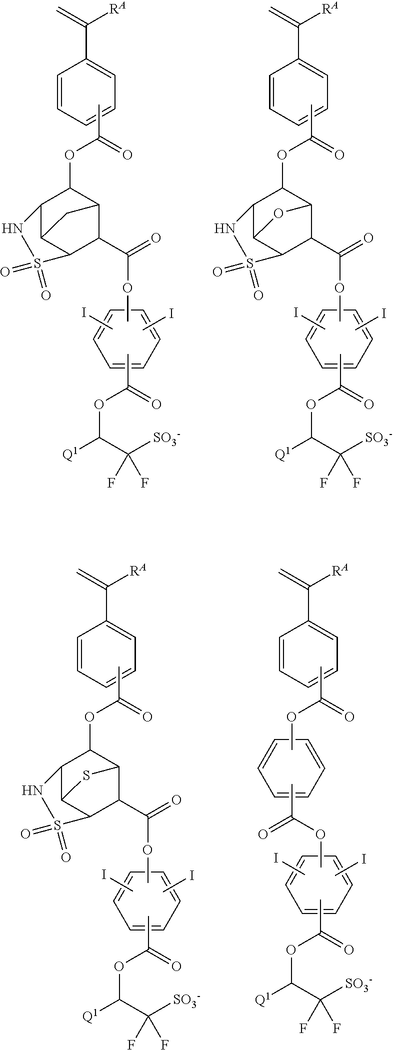

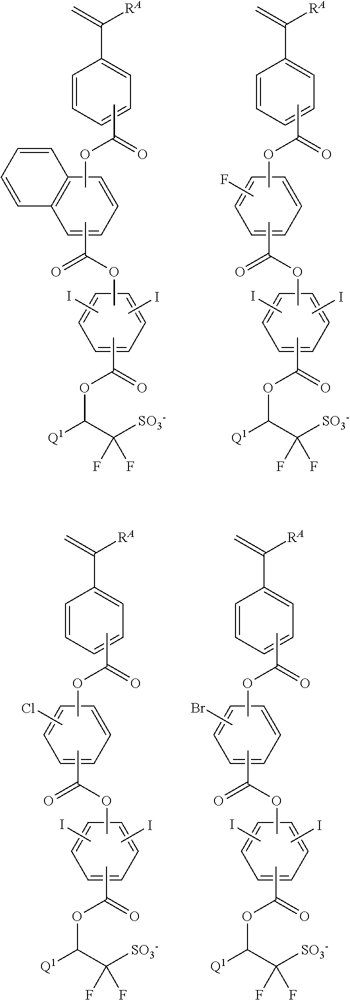

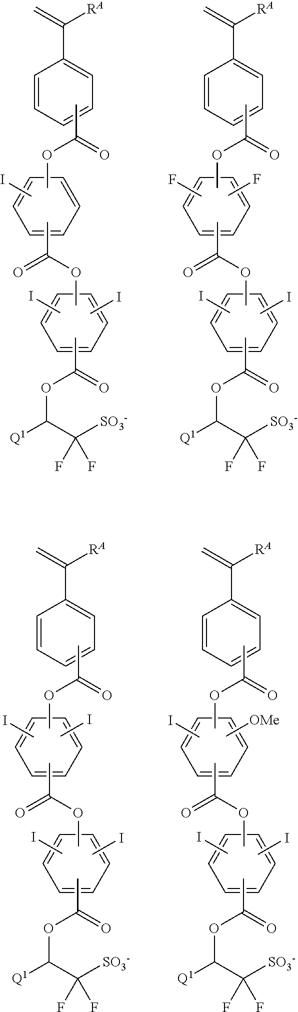

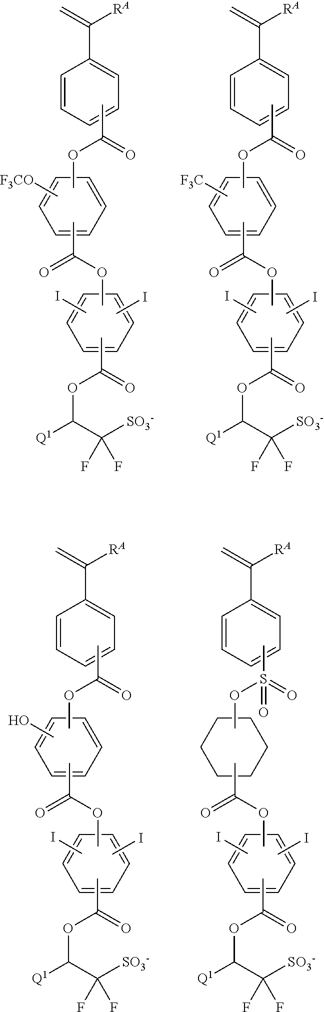

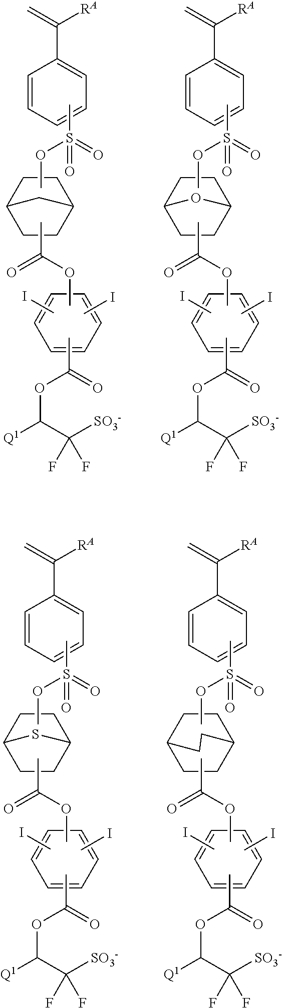

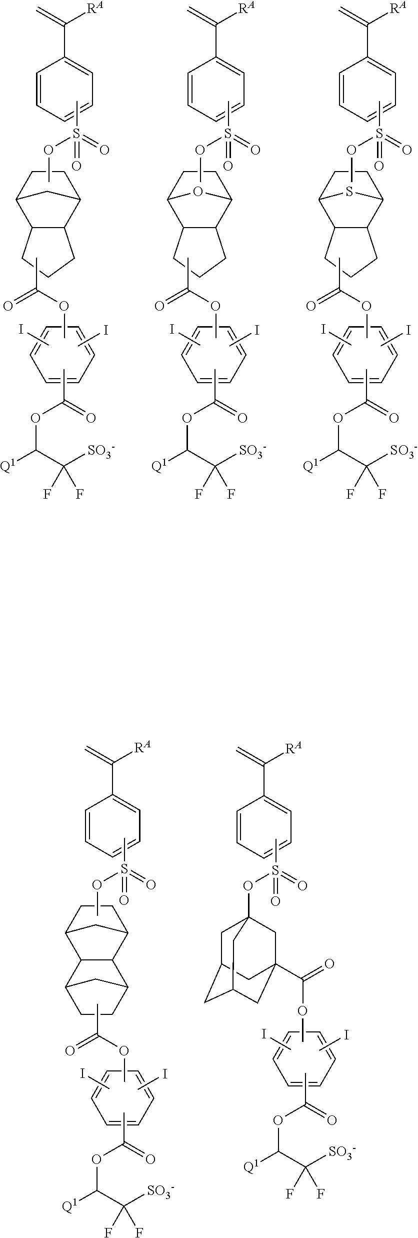

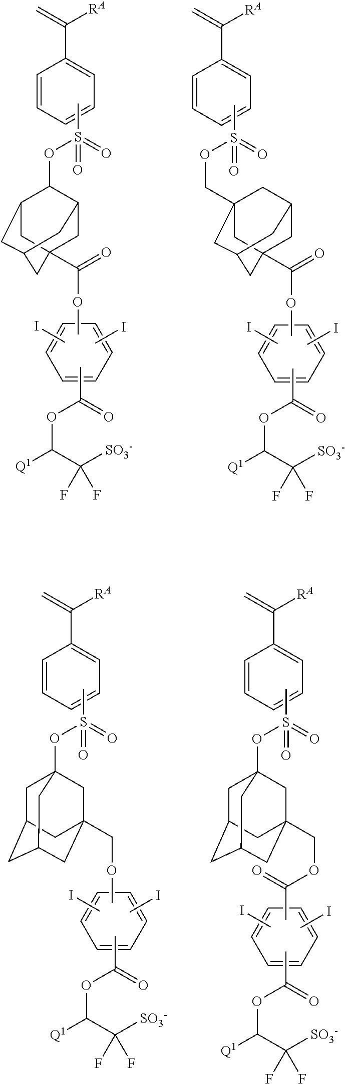

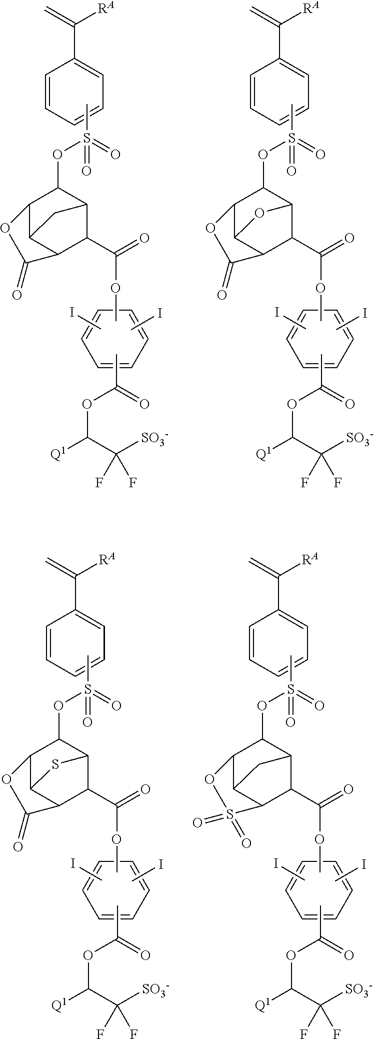

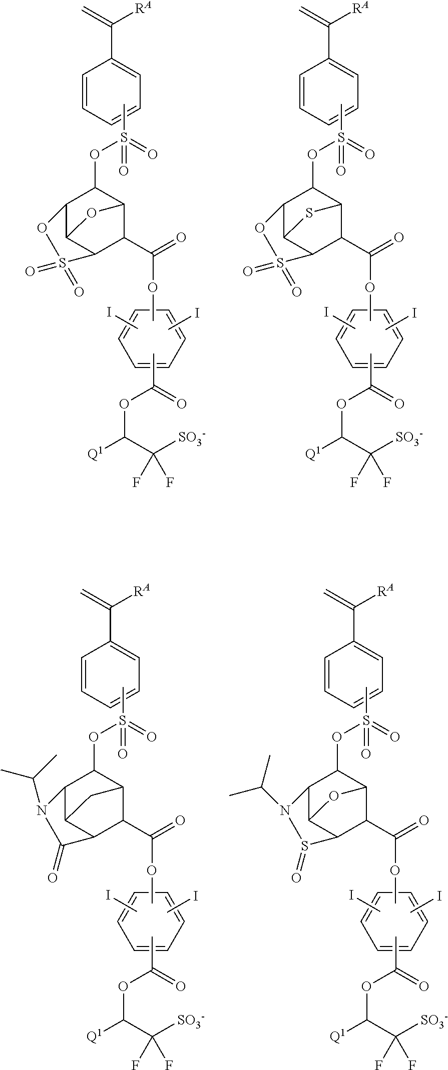

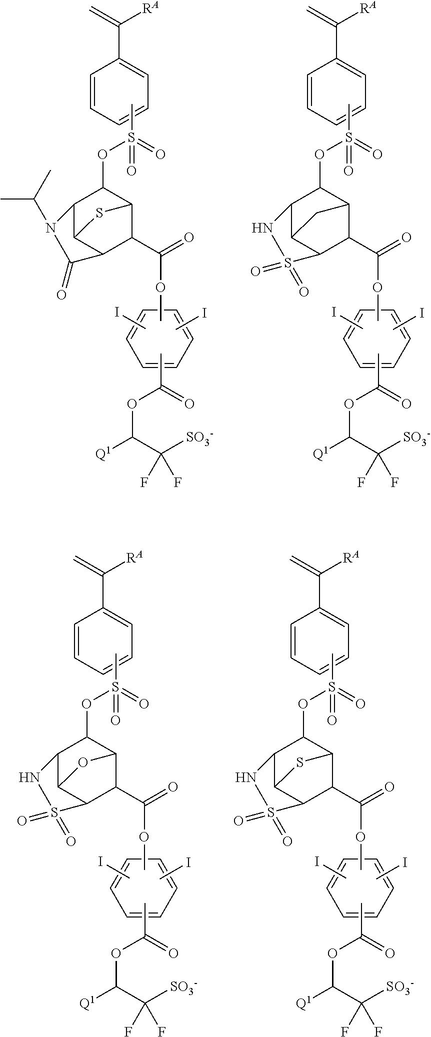

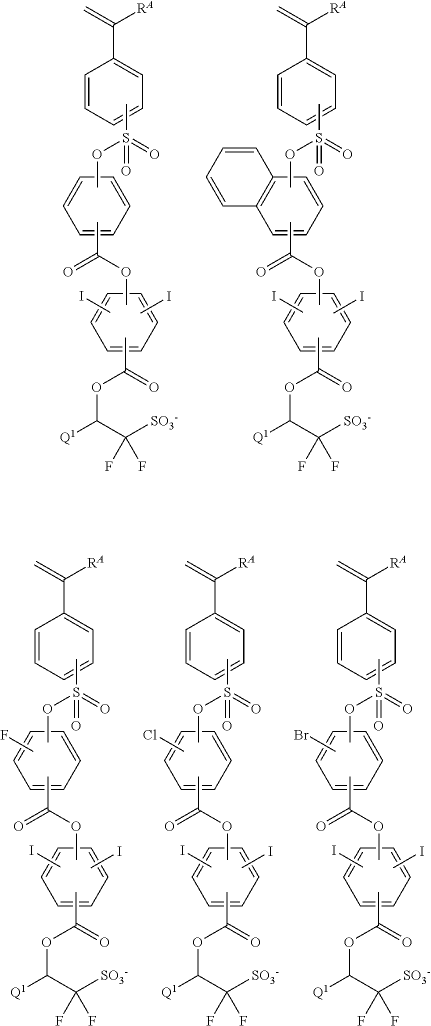

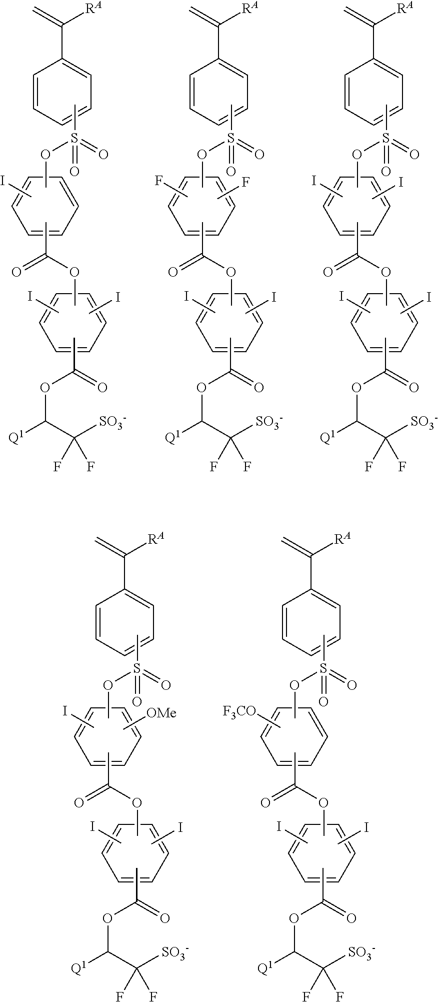

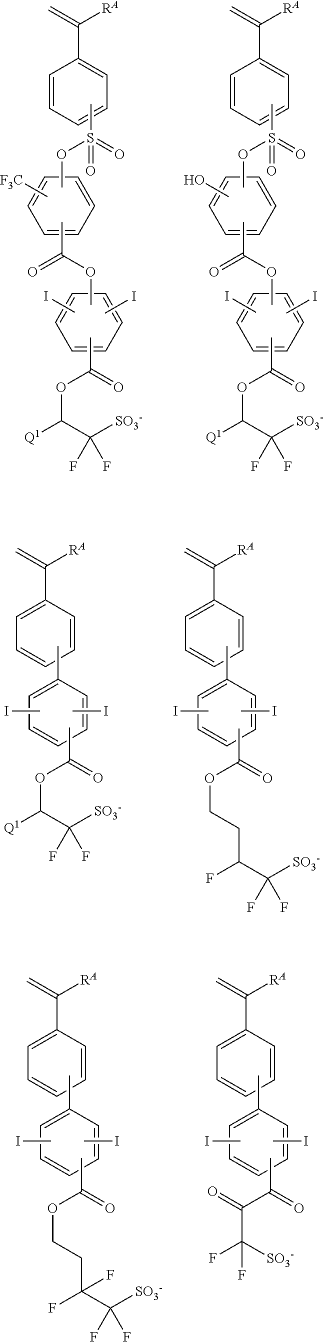

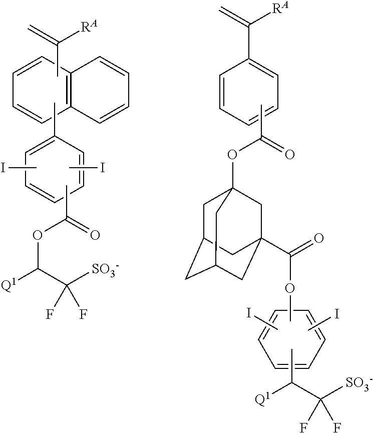

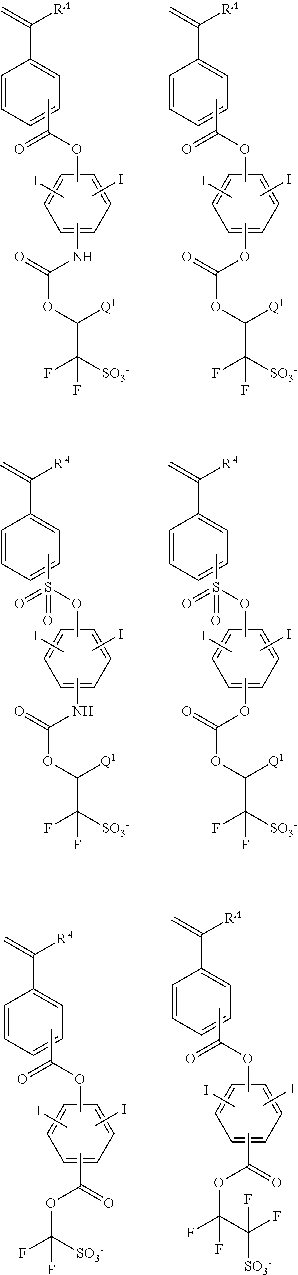

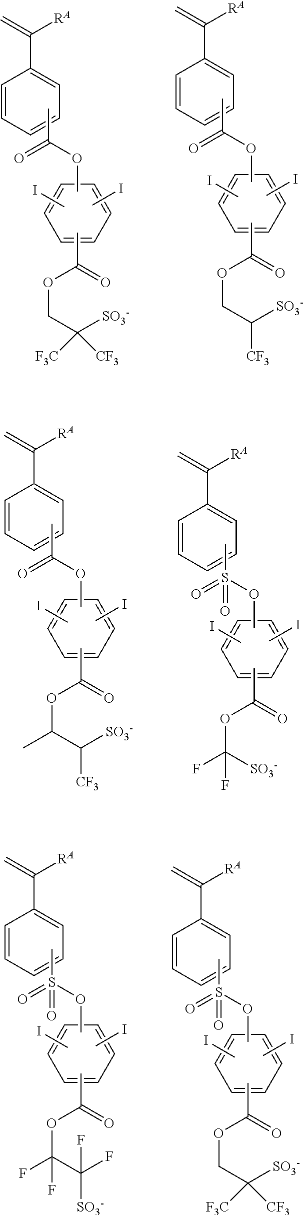

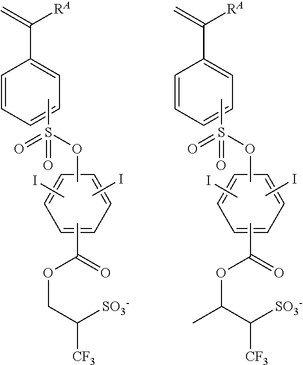

Furthermore, the anion of repeating unit d is preferably one represented by the following general formula (d2).

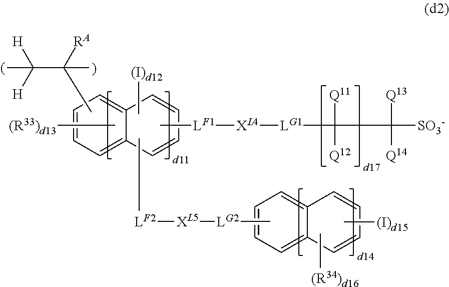

In the general formula (d2), d11 is 0 or 1. While a benzene ring is given if d11 is 0, and a naphthalene ring is given if d11 is 1, the case that d11 is 0 to give a benzene ring is preferred for solubility in solvents. d12 is 1, 2, 3, or 4. d12 is preferably 1, 2, or 3, more preferably 1 or 2, and further preferably 1 for raw material procurement. d13 is 0, 1, or 2. It should be noted that 1≤d12+d13≤4 is satisfied if d11 is 0, and 1≤d12+d13≤6 is satisfied if d11 is 1.

In the general formula (d2), d14 is 0 or 1. While a benzene ring is given if d14 is 0, and a naphthalene ring is given if d14 is 1, the case that d14 is 0 to give a benzene ring is preferred for solubility in solvents. d15 is 1, 2, 3, or 4, and preferably 1, 2, or 3. More iodine atoms in the anion structure result in enhanced absorption particularly to EUV, but in poor solubility in solvents, which may disadvantageously cause precipitation in the resist composition, and for this reason the number of iodine atoms in the anion is preferably 2, 3, 4, or 5, and further preferably 2, 3, or 4. d16 is 0, 1, or 2. It should be noted that 1≤d15+d16≤4 is satisfied if d14 is 0, and 1≤d15+d16≤6 is satisfied if d14 is 1.

In the general formula (d2), d17 is 0, 1, 2, 3, or 4, preferably 0, 1, 2, or 3, more preferably 1, 2, or 3, and further preferably 1.

In the general formula (d2), R^{A} is a hydrogen atom, a fluorine atom, a methyl group, or a trifluoromethyl group. Among them, R^{A} is preferably a hydrogen atom or a methyl group, and more preferably a hydrogen atom.

In the general formula (d2), R³³ is a halogen atom being not an iodine atom, a nitro group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. The halogen atom being not an iodine atom is preferably a fluorine atom, a chlorine atom, or a bromine atom, and more preferably a fluorine atom. The hydrocarbyl group and the hydrocarbyl moieties of the hydrocarbyloxy group and hydrocarbylthio group may be each saturated or unsaturated, and may be each linear, branched, or cyclic. Specific examples thereof include an alkyl group having 1 to 20 carbon atoms such as a methyl group, an ethyl group, a n-propyl group, an isopropyl group, a n-butyl group, an isobutyl group, a sec-butyl group, a tert-butyl group, a n-pentyl group, a n-hexyl group, a n-octyl group, a n-nonyl group, a n-decyl group, an undecyl group, a dodecyl group, a tridecyl group, a tetradecyl group, a pentadecyl group, a heptadecyl group, an octadecyl group, a nonadecyl group, and an icosyl group; a cyclic saturated hydrocarbyl group having 3 to 20 carbon atoms such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; an alkenyl group having 2 to 20 carbon atoms such as a vinyl group, an allyl group, a propenyl group, a butenyl group, and a hexenyl group; a cyclic unsaturated hydrocarbyl group having 3 to 20 carbon atoms such as a cyclohexenyl group; an aryl group having 6 to 20 carbon atoms such as a phenyl group and a naphthyl group; an aralkyl group having 7 to 20 carbon atoms such as a benzyl group, a 1-phenylethyl group, and a 2-phenylethyl group; and a group given by combining any of these. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained. If d13 is 2, R³³ groups are identical to or different from each other.

If d13 is 2, the two R³³ groups are optionally bound together to form a ring together with the carbon atoms of the aromatic ring or aromatic rings to which the two groups are bound. Specific examples of the ring formed in this case include a cyclopropane ring, a cyclobutane ring, a cyclopentane ring, a cyclohexane ring, a norbornane ring, and an adamantane ring. Some or all of the hydrogen atoms in the ring may be each replaced with a group containing a heteroatom such as an oxygen atom, a sulfur atom, a nitrogen atom, and a halogen atom, some of the —CH₂— moieties in the ring may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

In the general formula (d2), R³⁴ is a halogen atom being not an iodine atom, a nitro group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. Specific examples of the halogen atom being not an iodine atom include a fluorine atom, a chlorine atom, and a bromine atom. The hydrocarbyl group and the hydrocarbyl moieties of the hydrocarbyloxy group and hydrocarbylthio group may be each saturated or unsaturated, and may be each linear, branched, or cyclic. Specific examples thereof include, but are not limited to, those shown as examples of the hydrocarbyl group represented by R³³. If d16 is 2, R³⁴ groups are identical to or different from each other.

If d16 is 2, the two R³⁴ groups are optionally bound together to form a ring together with the carbon atoms to which the two groups are bound. The ring is preferably a 5- to 8-membered ring.

In the general formula (d2), L^{F1}, L^{F2}, L^{G1}, and L^{G2} are each independently a single bond, an ether bond, an ester bond, a sulfonate bond, an amide bond, a sulfonamide bond, a carbonate bond, or a carbamate bond. Among them, L^{F1} is preferably a single bond, an ether bond, an ester bond, or a sulfonate bond, and more preferably an ester bond or a sulfonate bond. L^{F2} is preferably a single bond, an ether bond, an ester bond, or a sulfonate bond, and more preferably an ester bond or a sulfonate bond. L^{G1} is preferably a single bond, an ether bond, an ester bond, or a sulfonate bond, and more preferably a single bond, an ether bond, or an ester bond. L^{G2} is preferably a single bond, an ether bond, an ester bond, or a sulfonate bond, and more preferably a single bond, an ether bond, or an ester bond.

L^{F1} and L^{F2} are preferably bound to adjacent carbon atoms in the aromatic ring or aromatic rings. In this case, the substituent containing the fluorosulfonate anion structure and the substituent containing an aromatic ring substituted with an iodine atom are present at positions spatially close to each other, and hence higher sensitivity is expected to be achieved.

In the general formula (d2), X^{L4} and X^{L5} are each independently a single bond or a hydrocarbylene group having 1 to 40 carbon atoms and optionally containing a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom. Specific examples of the hydrocarbylene group represented by X^{L4} and X^{L}, having 1 to 40 carbon atoms and optionally containing a heteroatom, include X^{L}-0 to X^{L}-58, which have been shown as specific examples of the hydrocarbylene group represented by X^{L1} and X^{L2}, having 1 to 40 carbon atoms and optionally containing a heteroatom, in the description of the formula (Z). Among them, X^{L}-0 to X^{L}-22, X^{L}-29 to X^{L}-34, and X^{L}-47 to X^{L}-58 are preferred.

In the general formula (d2), Q¹¹ and Q¹² are each independently a hydrogen atom, a fluorine atom, or a fluorinated saturated hydrocarbyl group having 1 to 6 carbon atoms. The fluorinated saturated hydrocarbyl group having 1 to 6 carbon atoms is preferably a trifluoromethyl group.

In the general formula (d2), Q¹³ and Q¹⁴ are each independently a fluorine atom or a fluorinated saturated hydrocarbyl group having 1 to 6 carbon atoms. The fluorinated saturated hydrocarbyl group having 1 to 6 carbon atoms is preferably a trifluoromethyl group. Q¹³ and Q¹⁴ are further preferably each a fluorine atom.

Specific examples of the partial structure represented by —[C(Q¹¹) (Q¹²)]_{d17}-C(Q¹³) (Q¹⁴)-SO₃— in the general formula (d2) are preferably those shown below, but are not limited thereto. In the following formulae, * indicates bonding to L^{G1}:

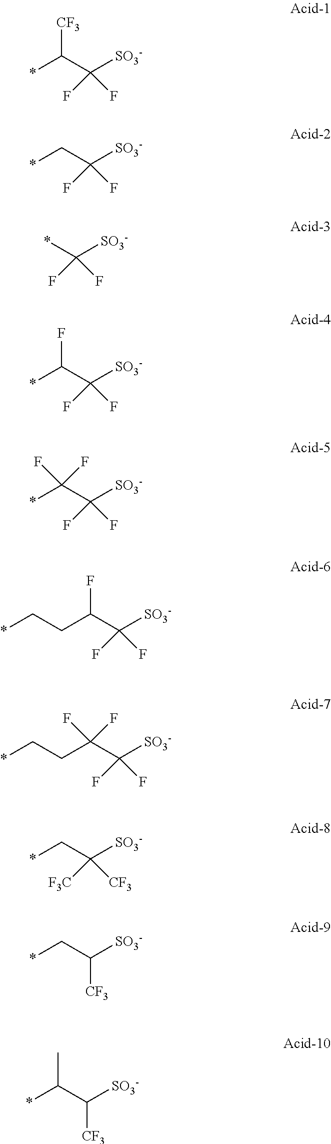

Among them, Acid-1 to Acid-7 are preferred, and Acid-1 to Acid-3, Acid-6, and Acid-7 are more preferred.

The anion represented by the general formula (d2) is preferably one represented by the following general formula (d2-1):

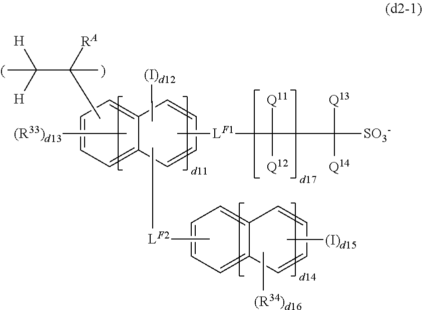

wherein R^{A}, R³³, R³⁴, L^{F1}, L^{F2}, Q¹¹ to Q¹⁴, and d11 to d17 are as specified above.

The anion represented by the general formula (d2-1) is preferably one represented by the following general formula (d2-2):

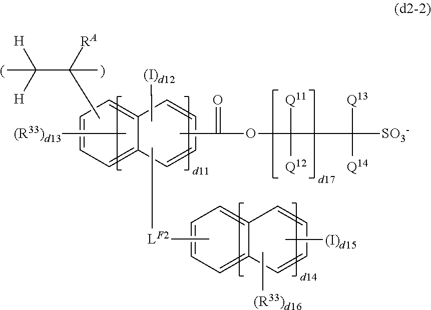

wherein R^{A}, R³³, R³⁴, L^{F2}, Q¹¹ to Q¹⁴, and d11 to d17 are as specified above.

Specific examples of the anion represented by the general formula (d2) include, but are not limited to, those shown below. In the following formulae, R^{A} and Q¹¹ are as specified above, and Me is a methyl group. The bonding positions of substituents in each aromatic ring may be interchanged therein.

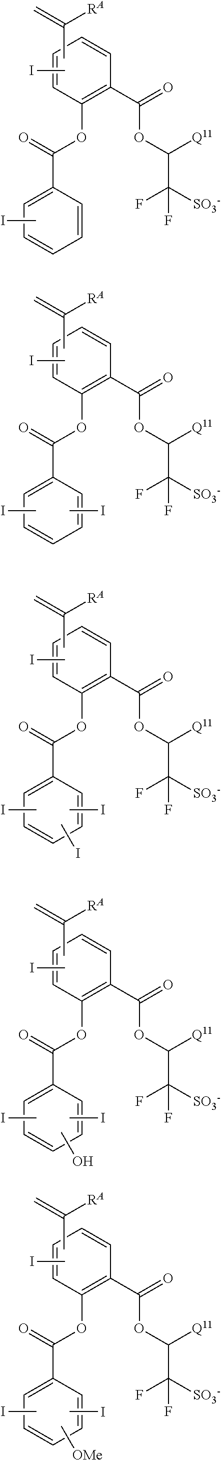

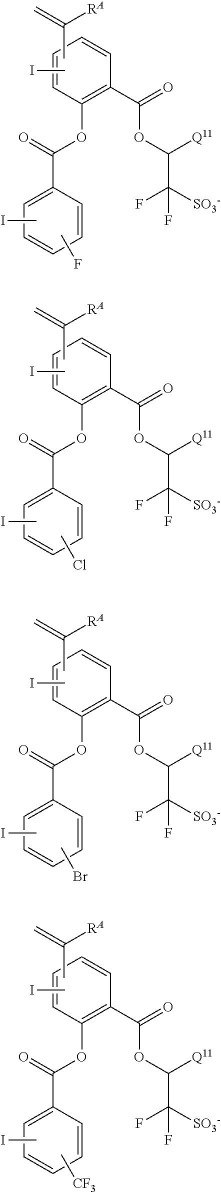

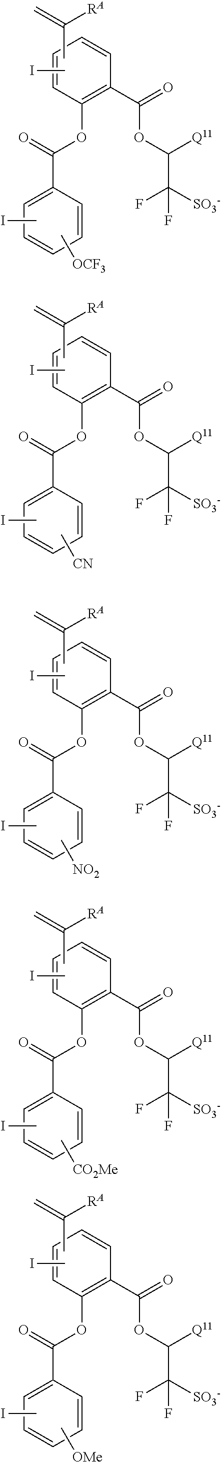

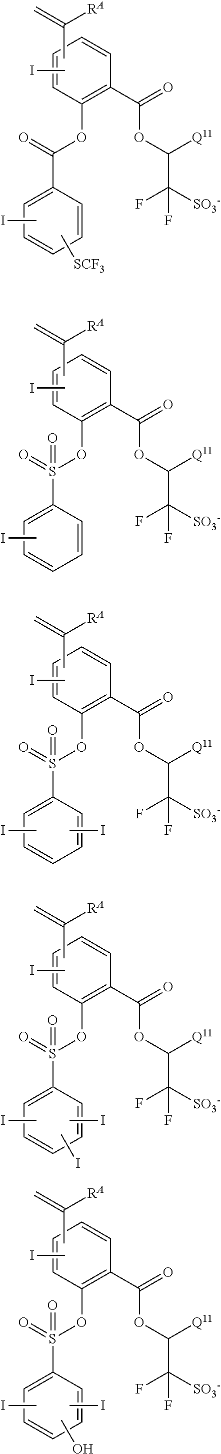

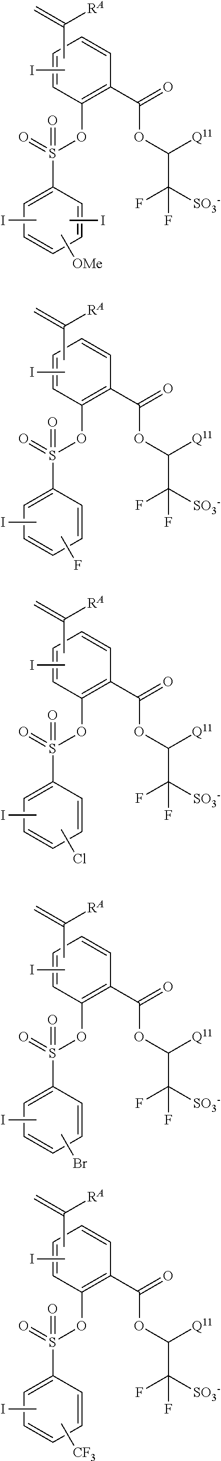

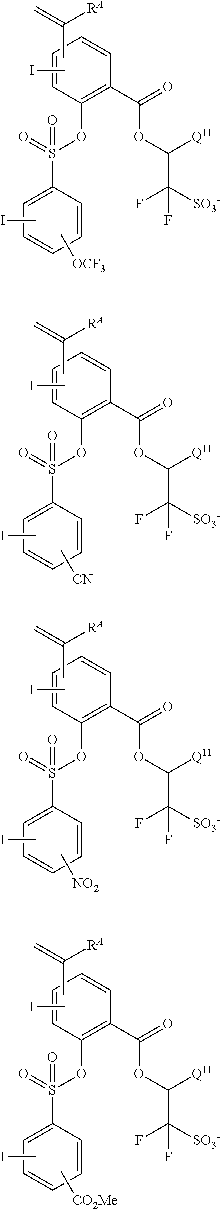

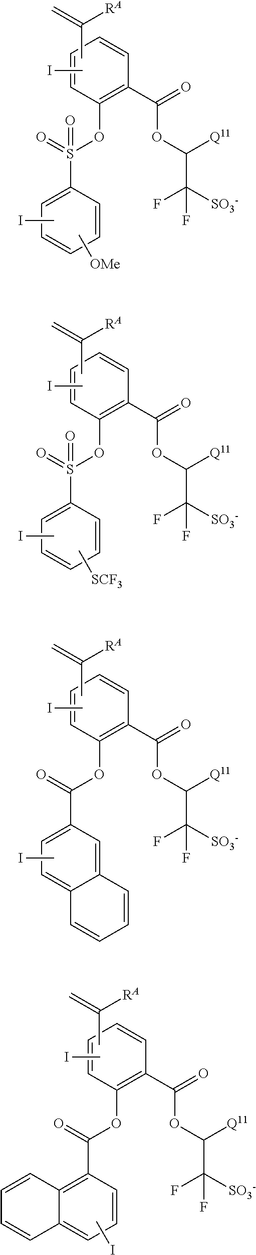

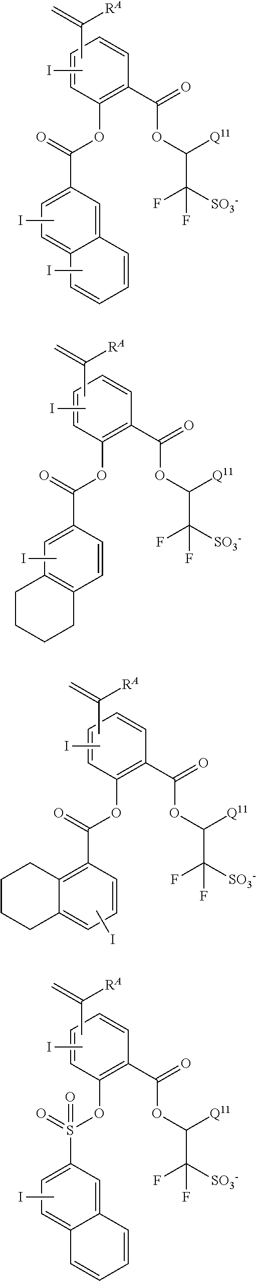

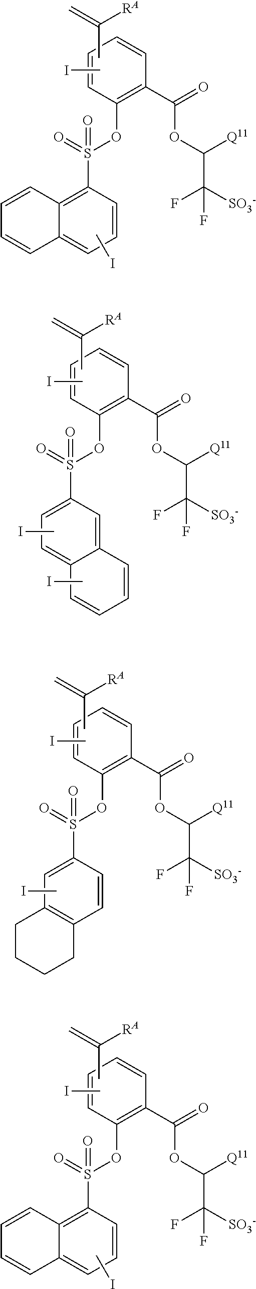

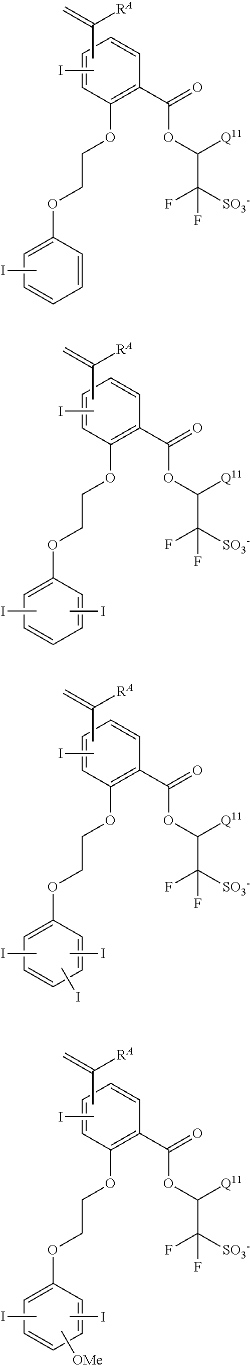

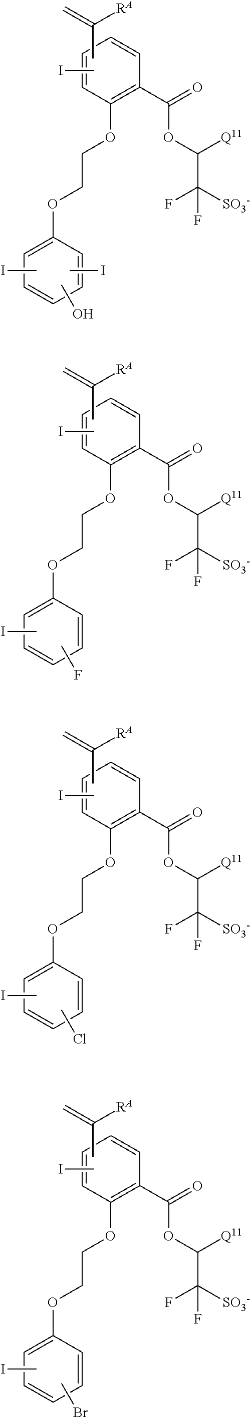

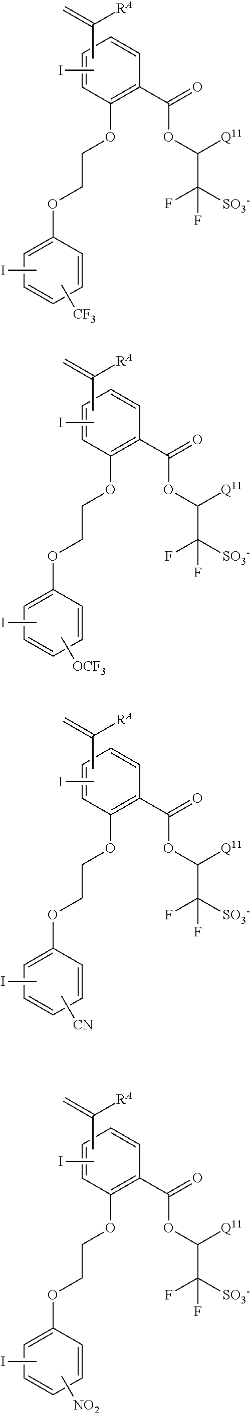

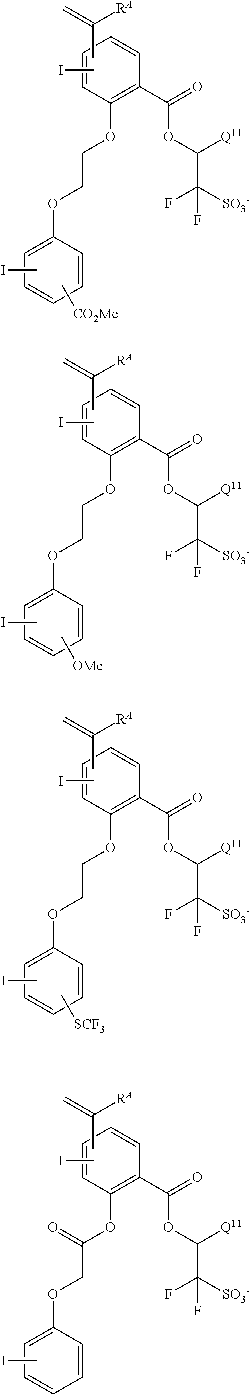

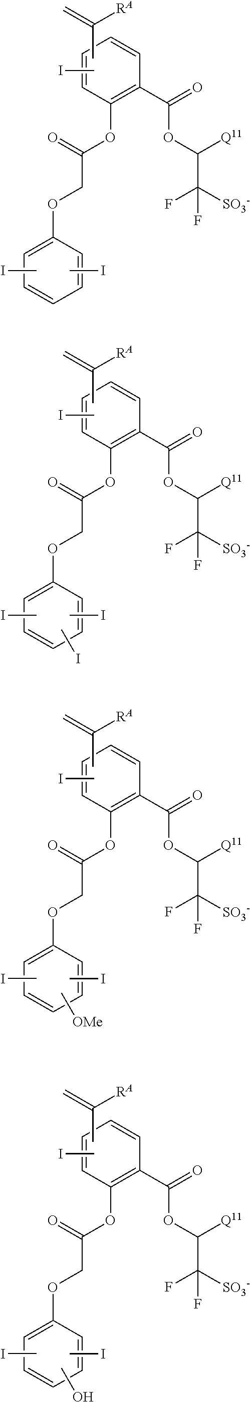

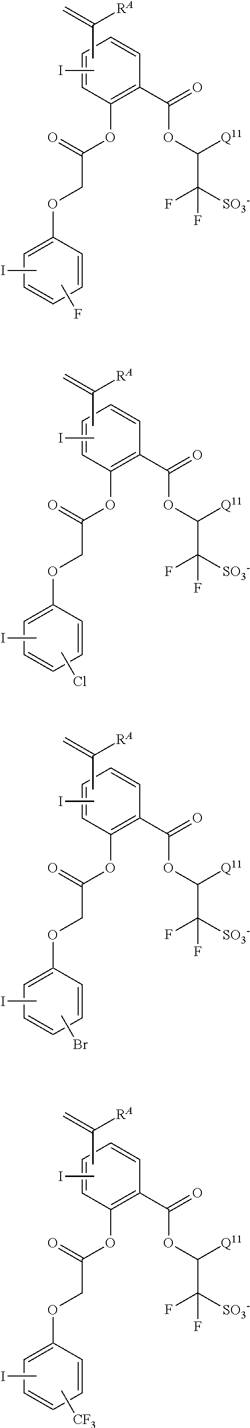

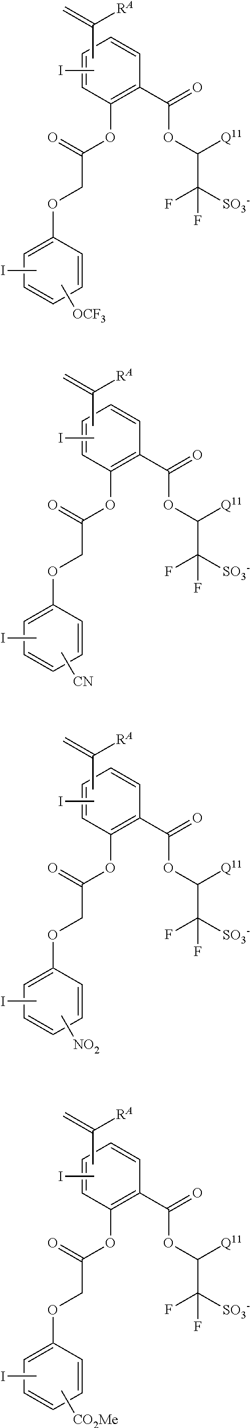

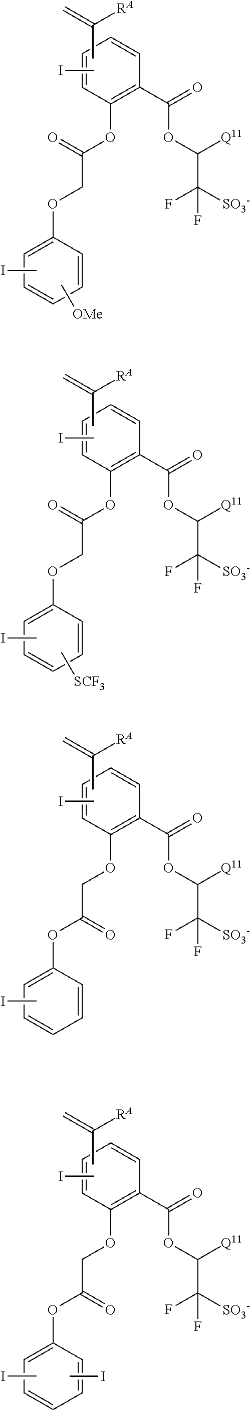

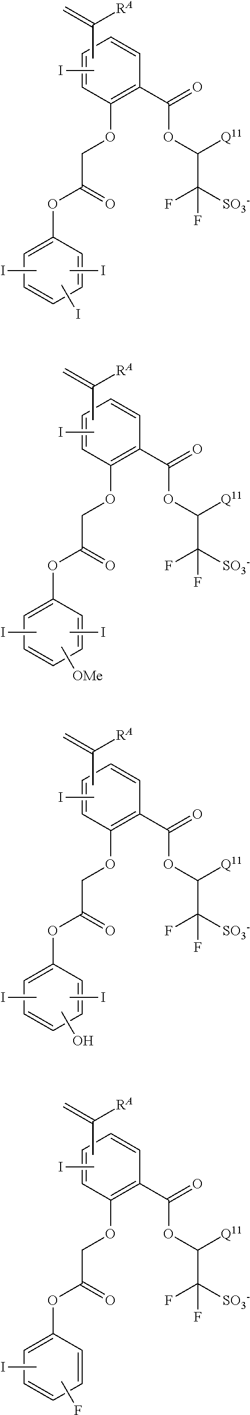

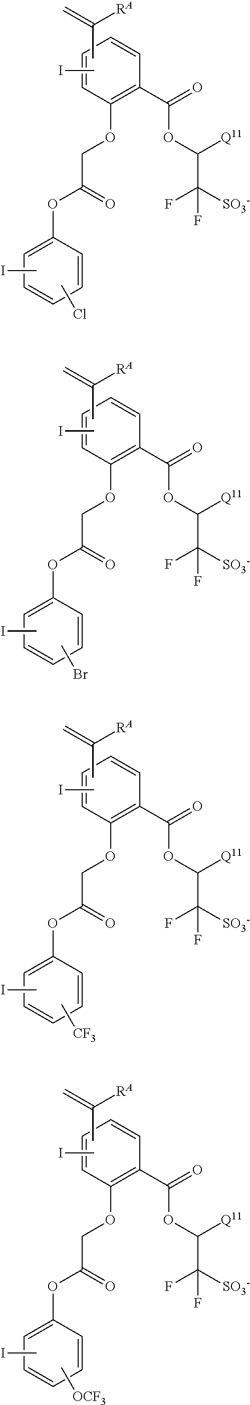

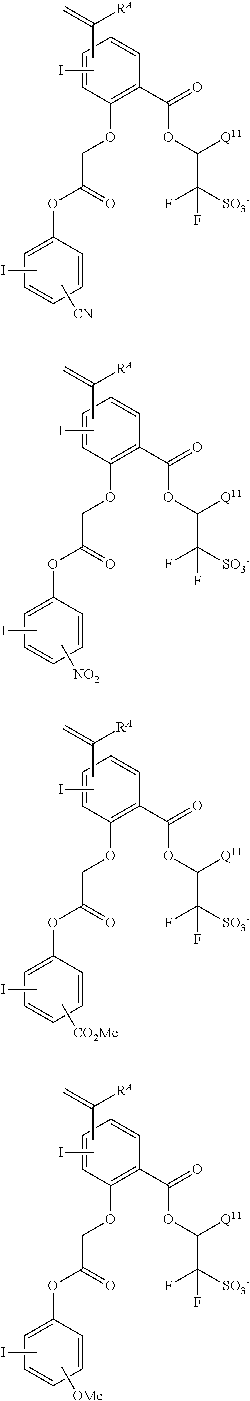

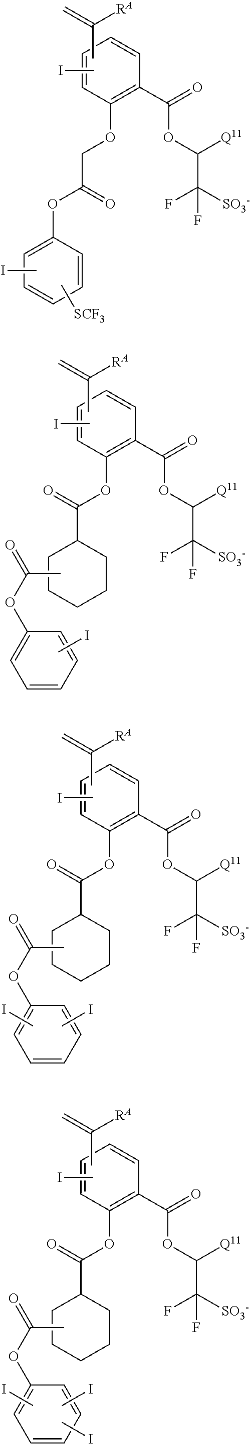

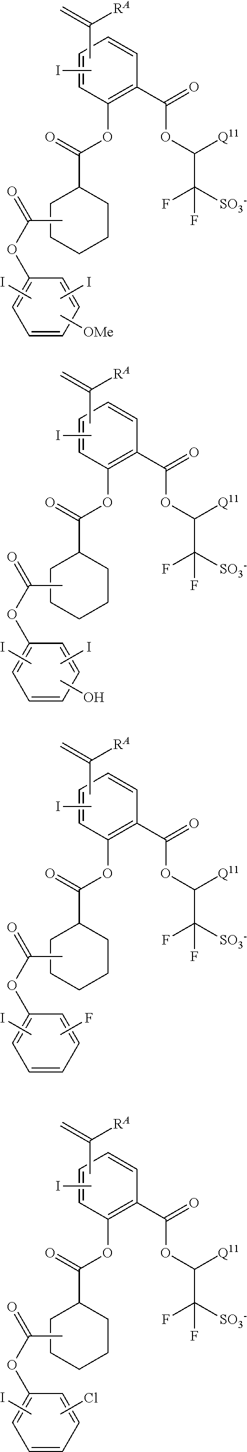

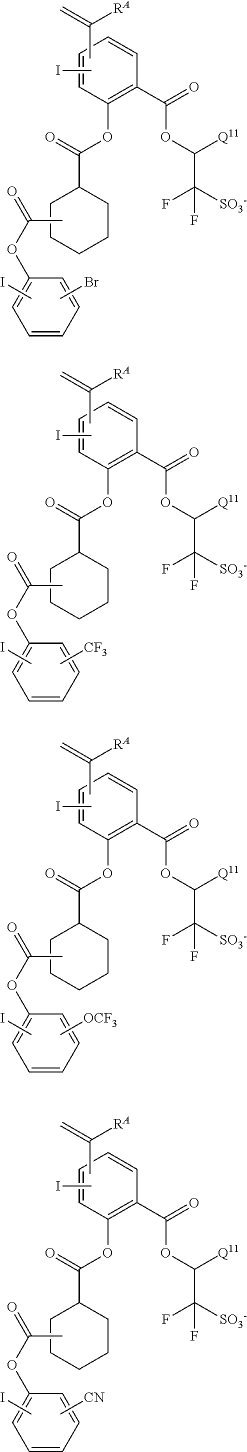

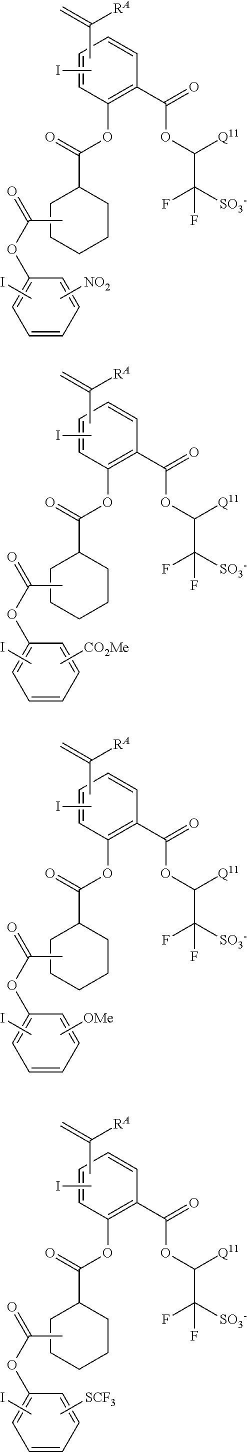

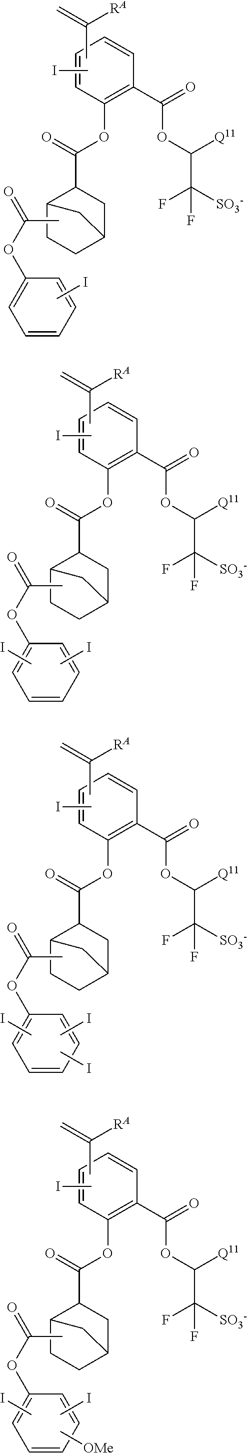

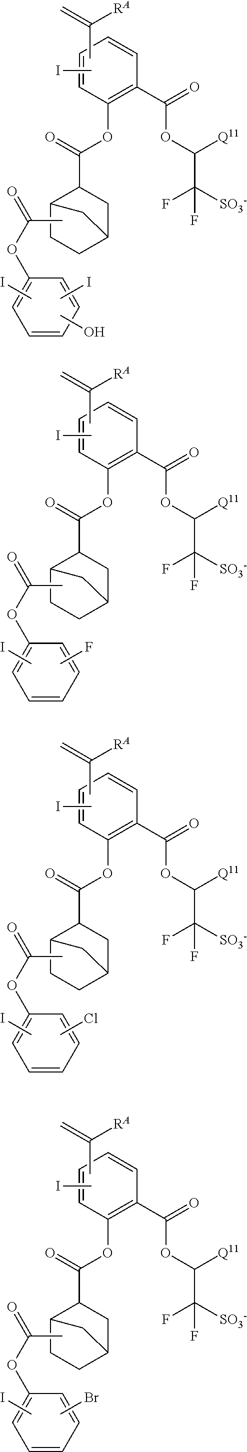

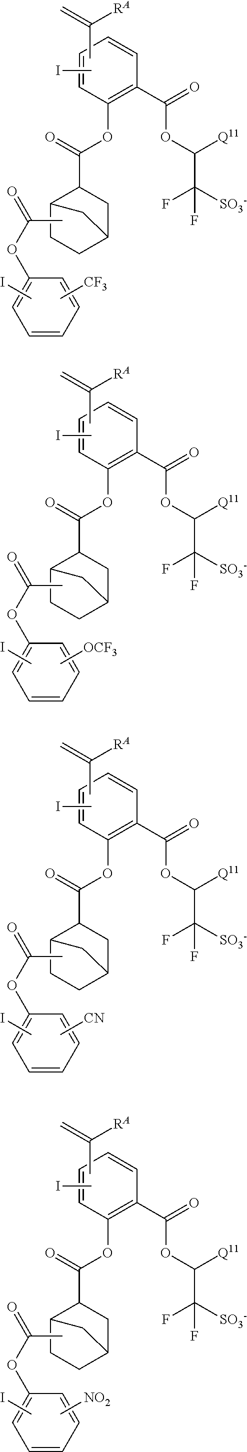

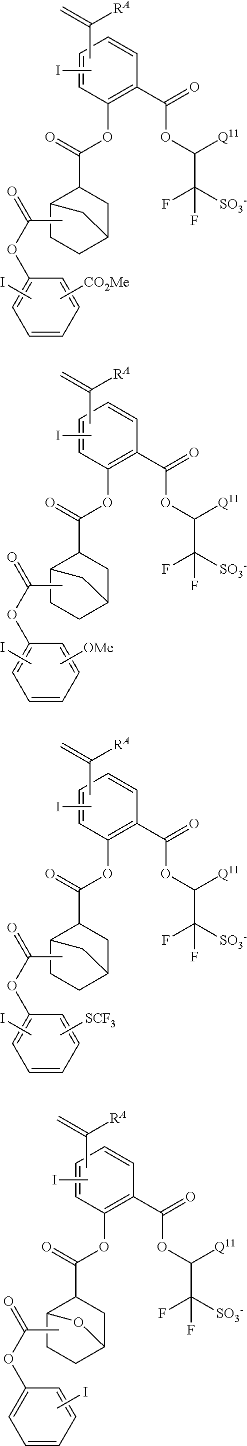

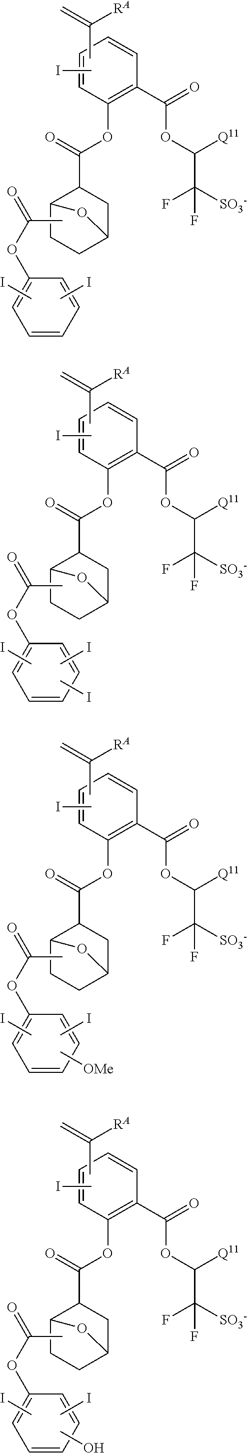

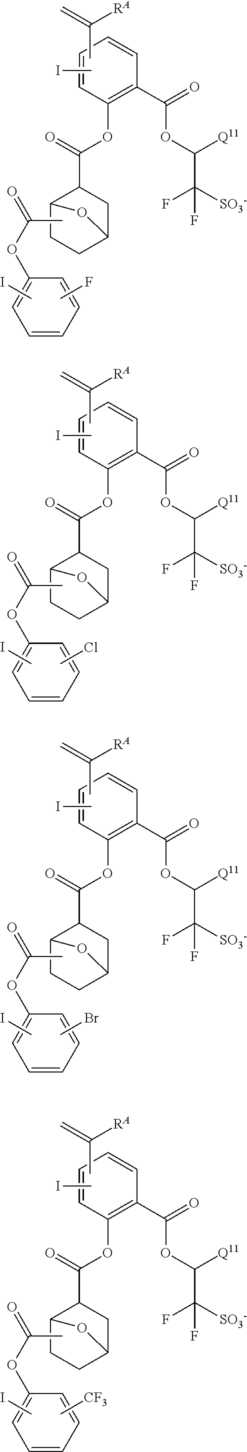

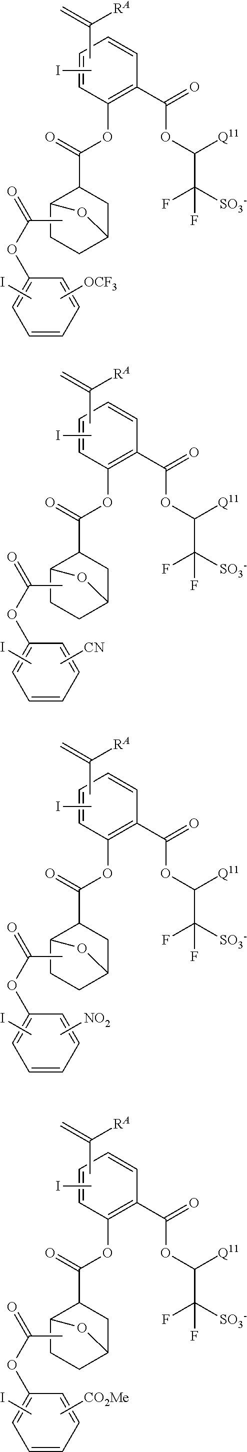

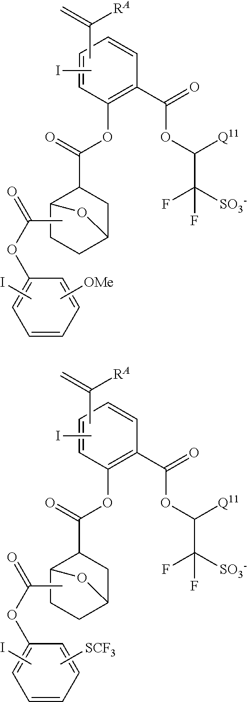

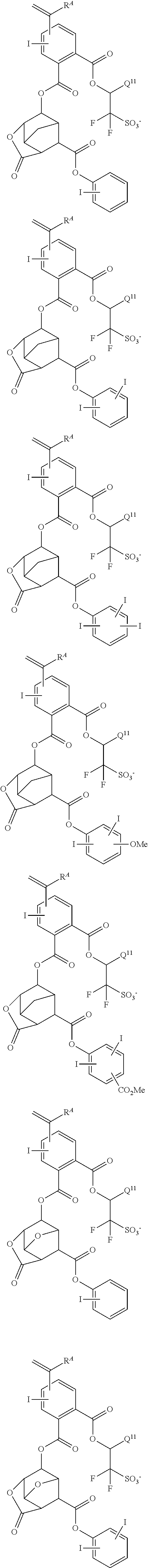

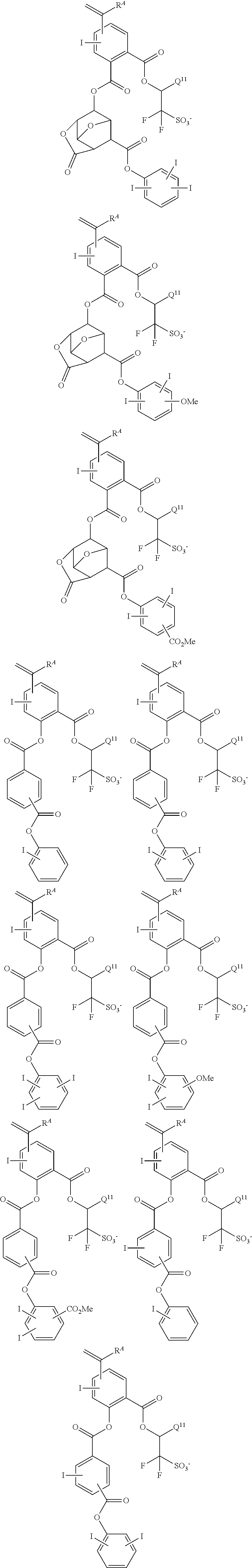

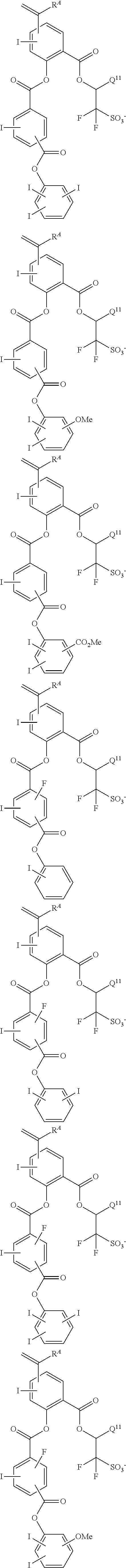

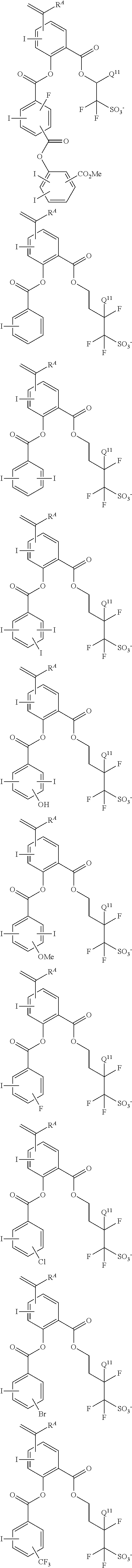

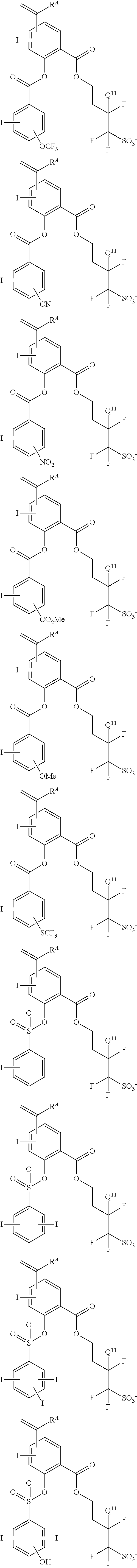

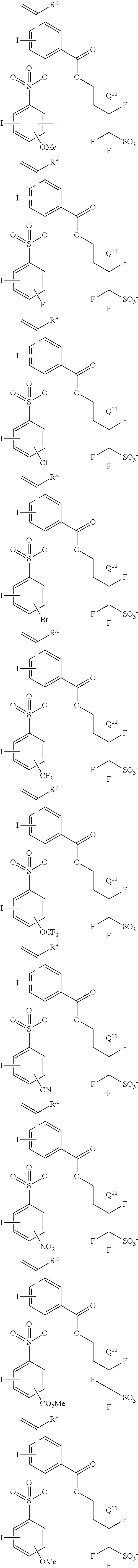

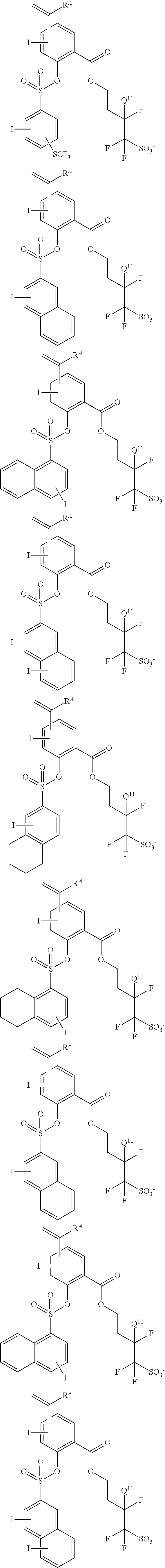

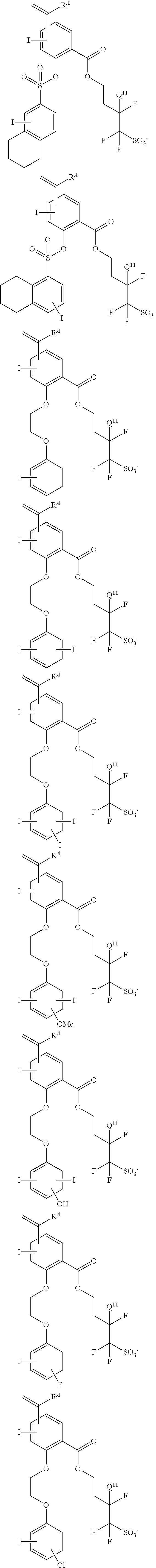

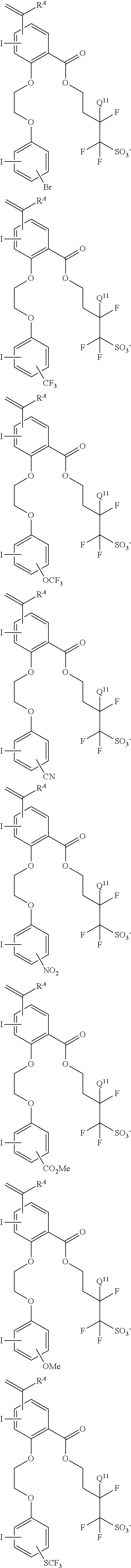

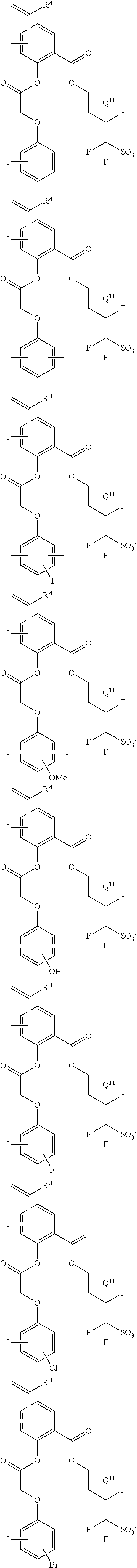

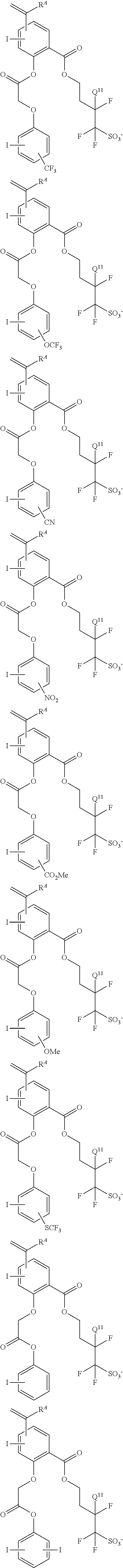

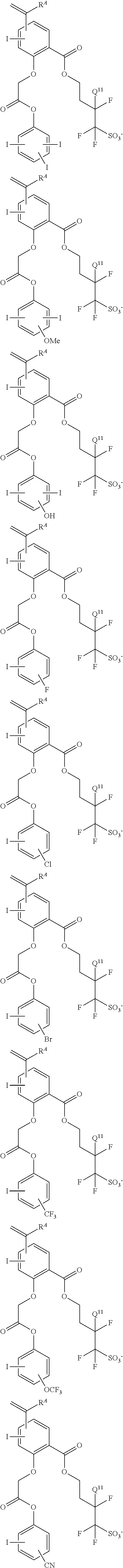

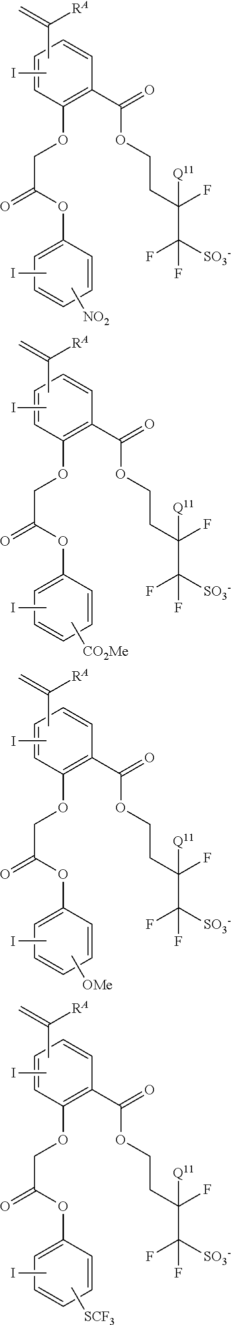

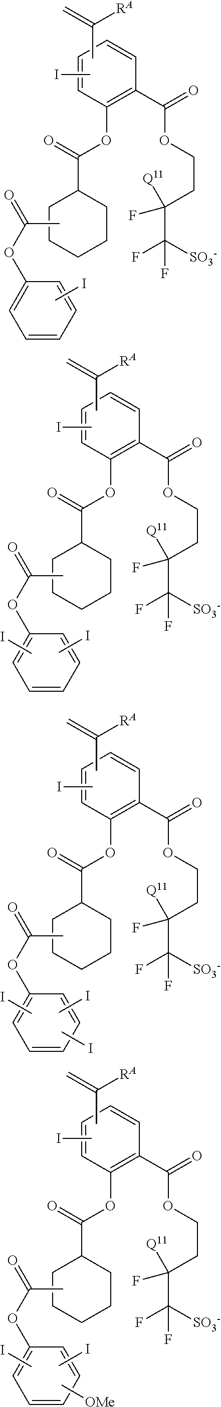

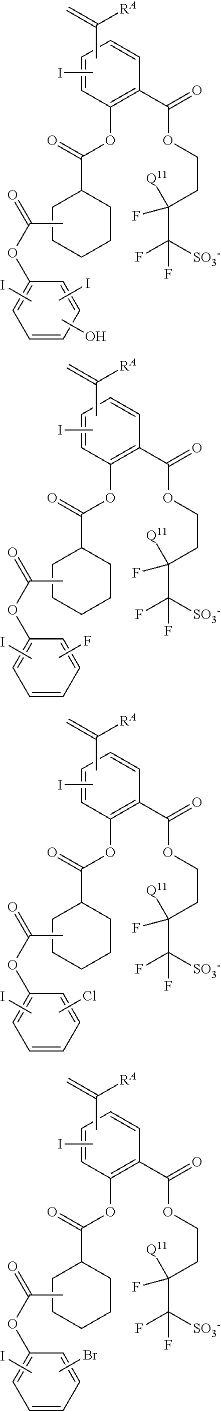

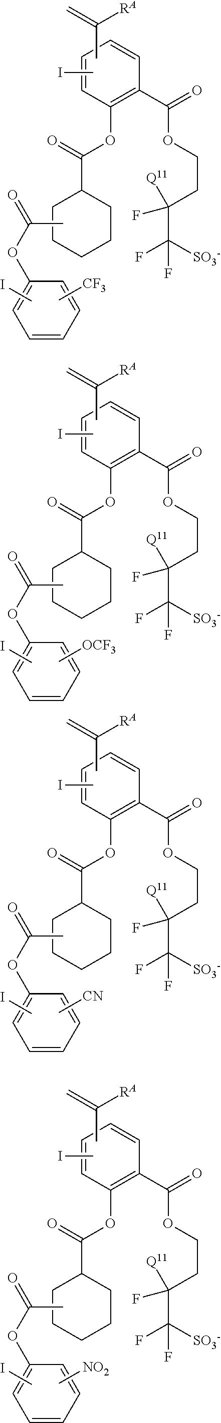

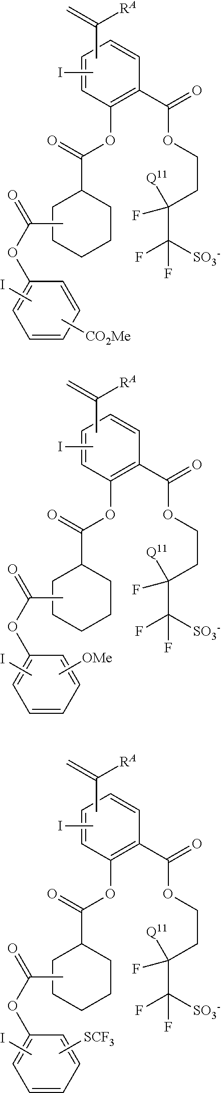

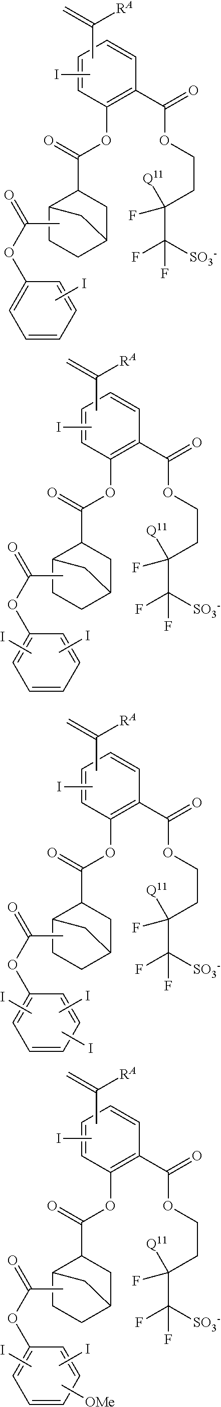

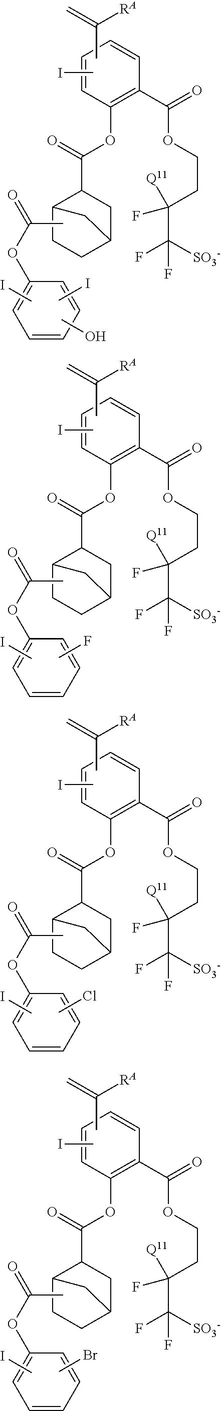

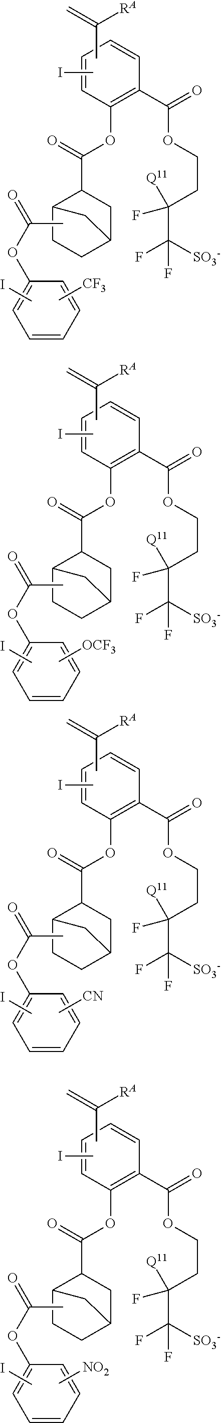

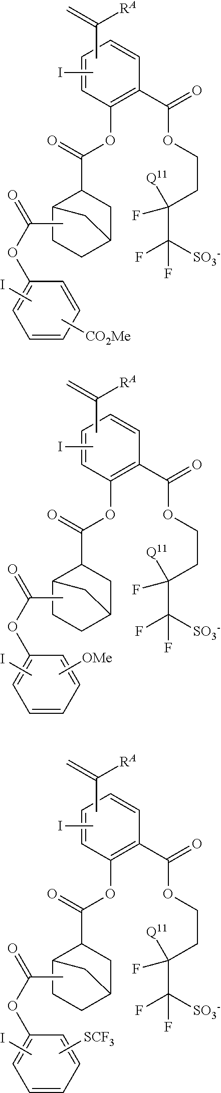

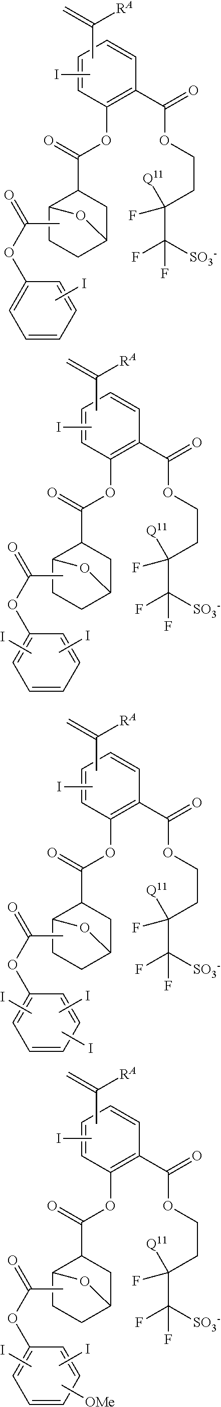

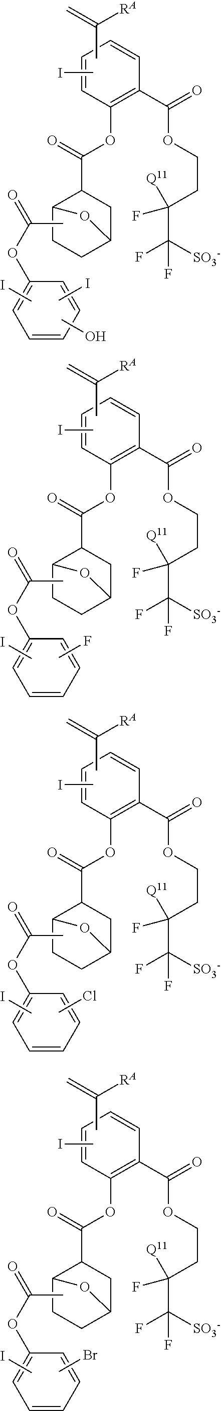

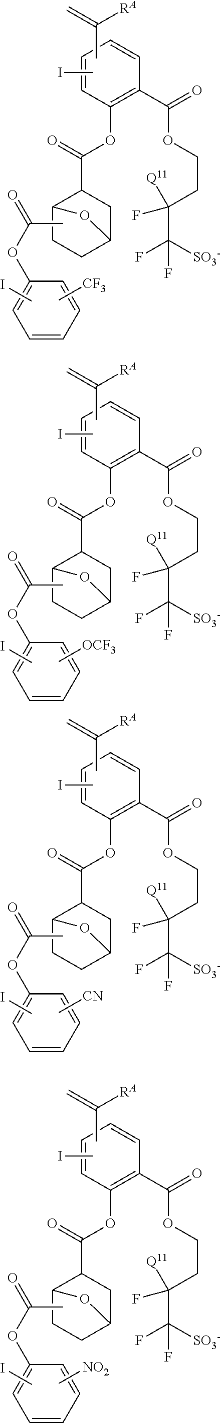

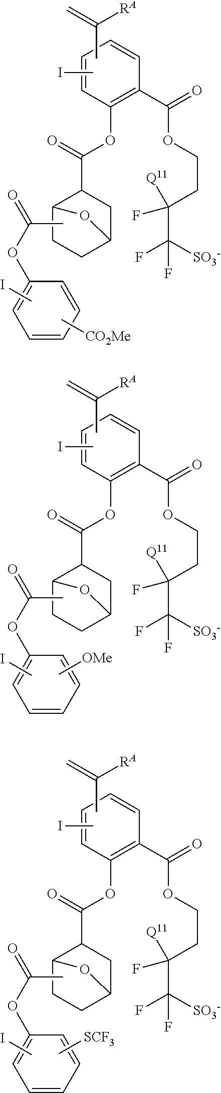

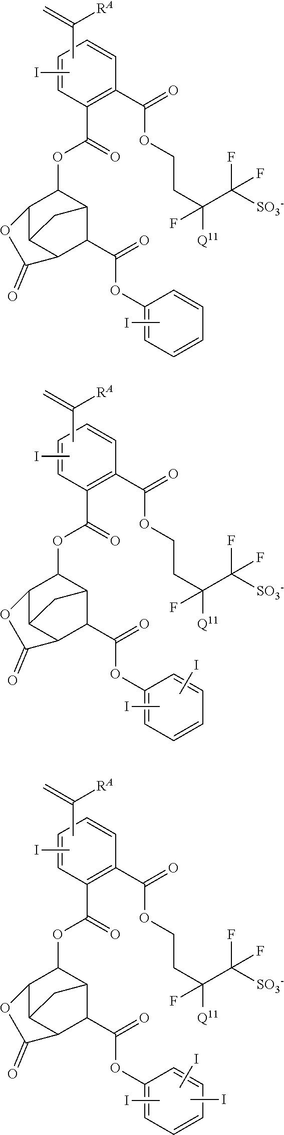

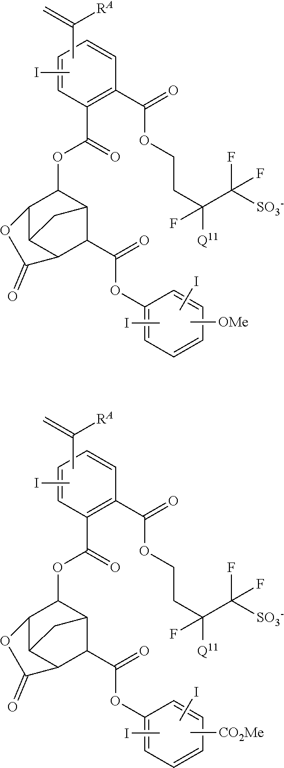

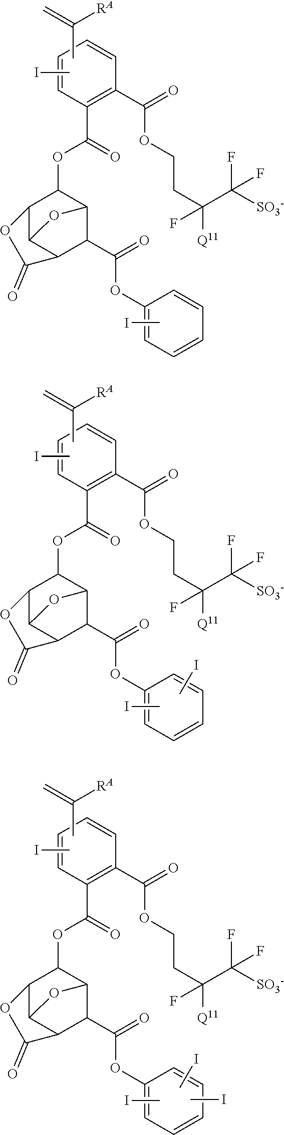

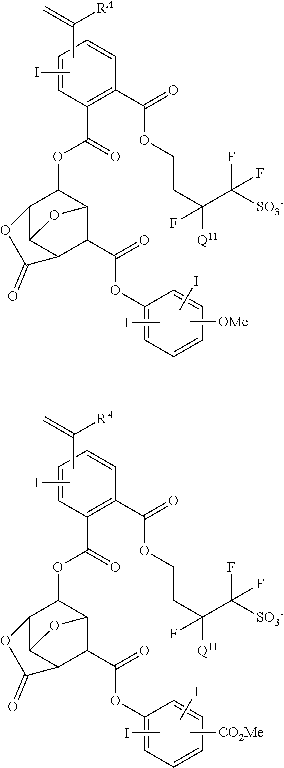

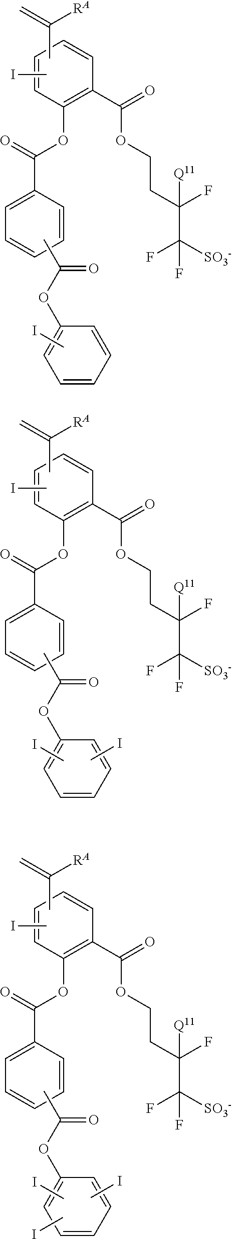

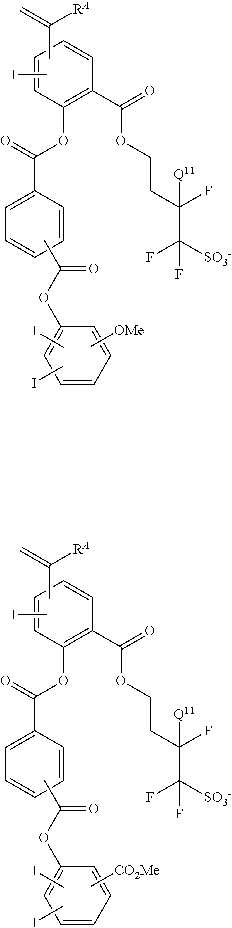

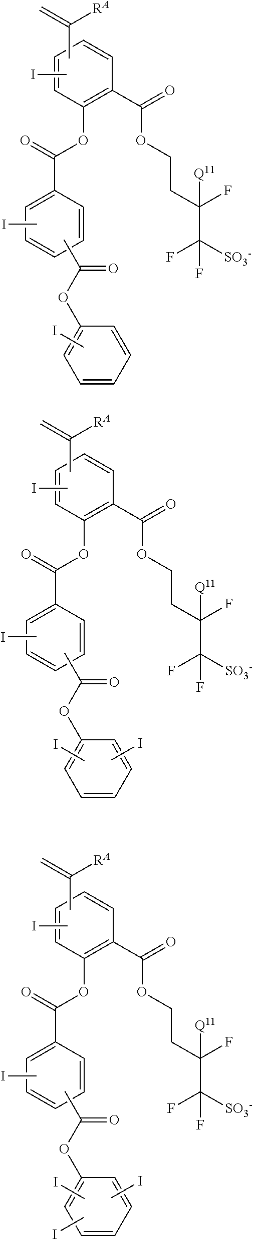

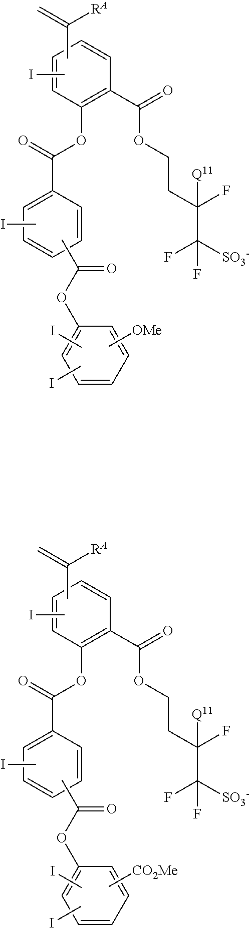

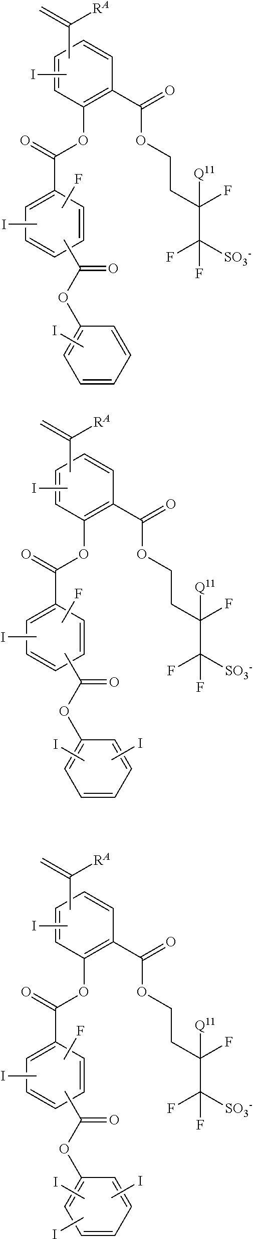

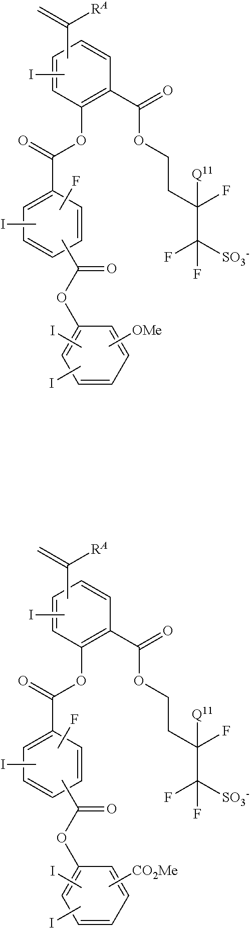

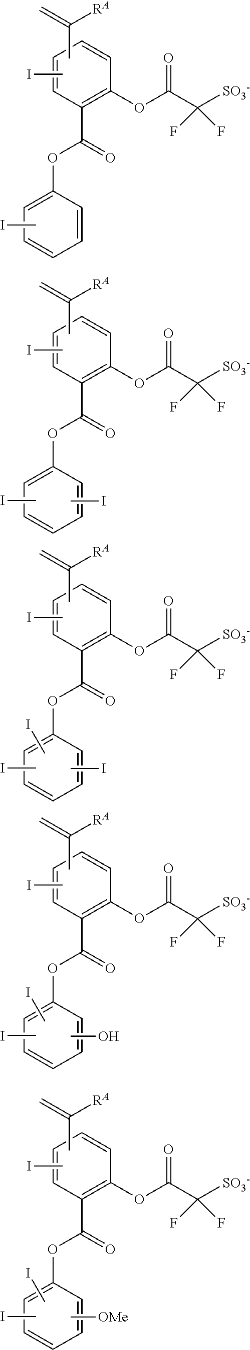

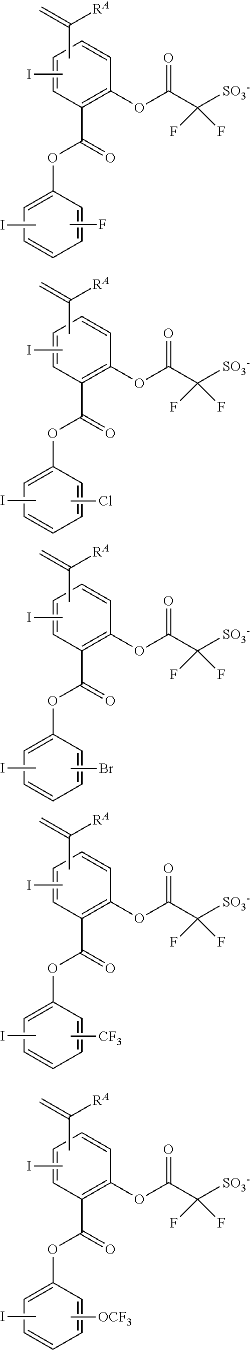

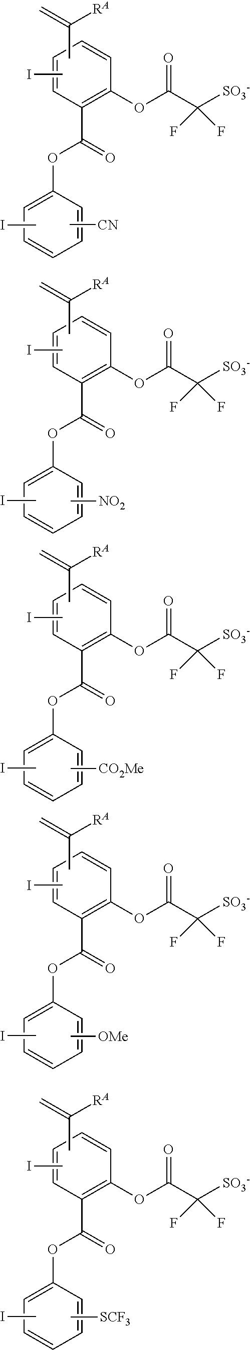

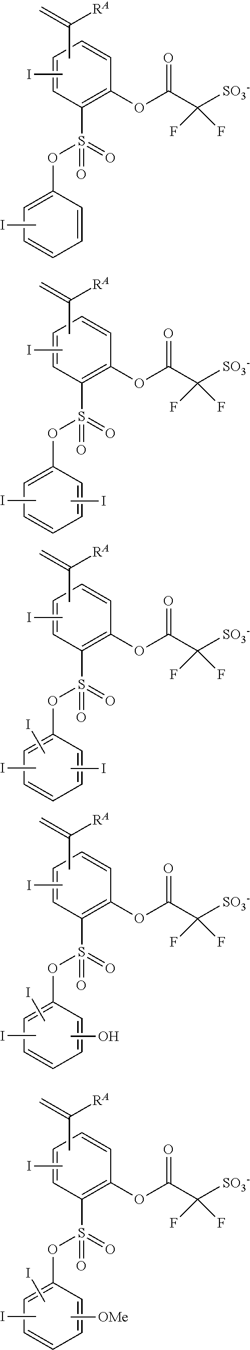

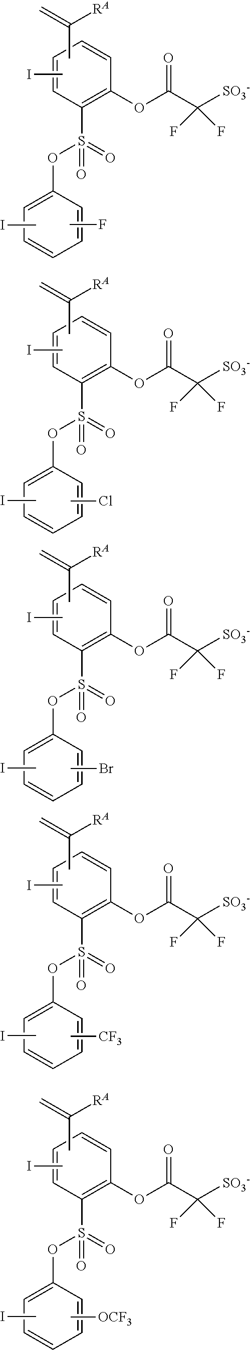

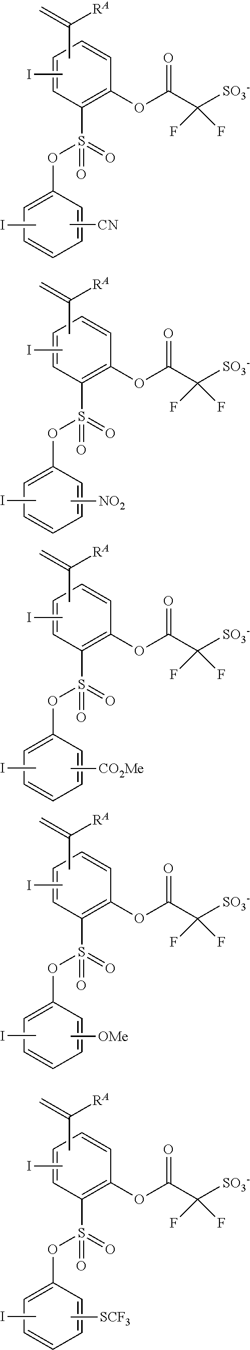

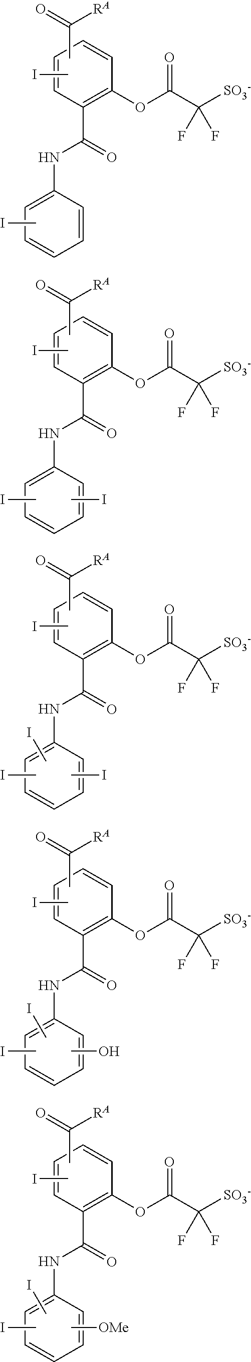

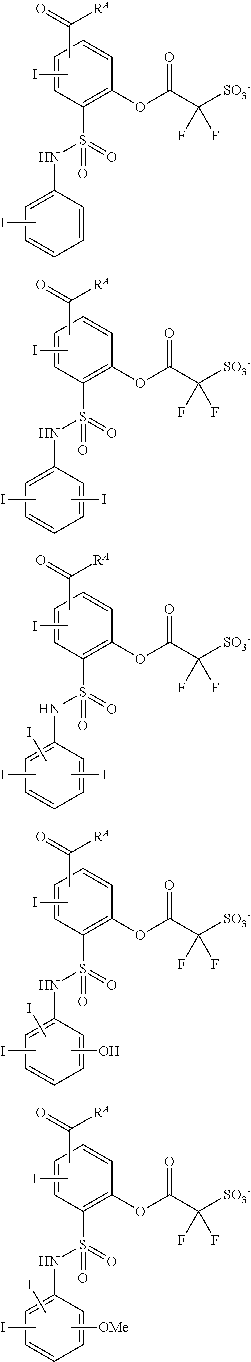

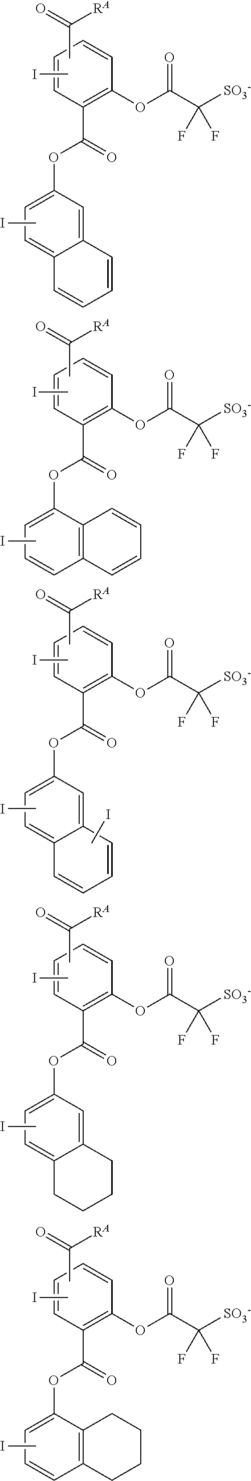

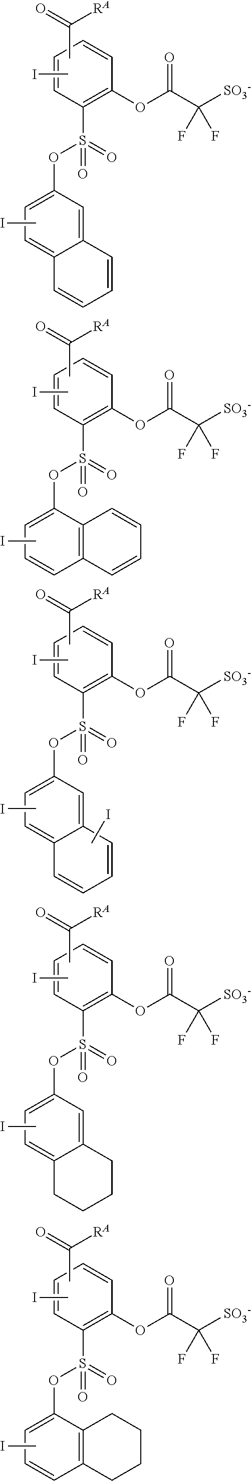

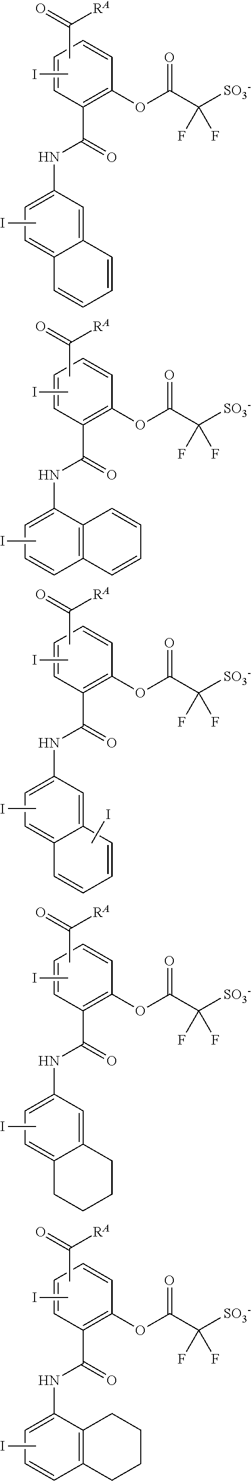

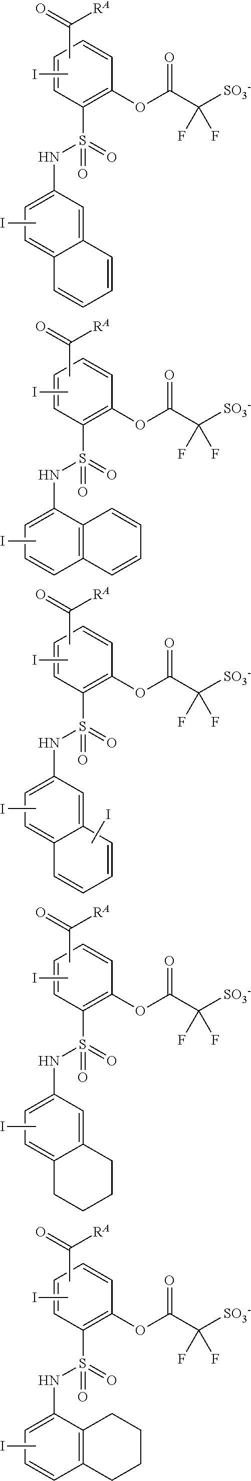

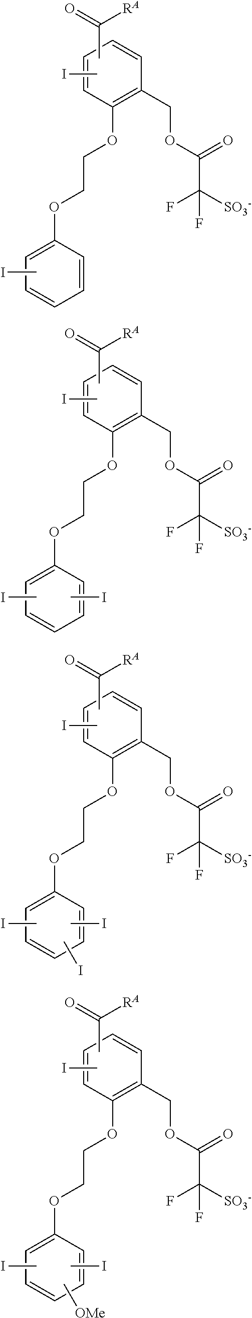

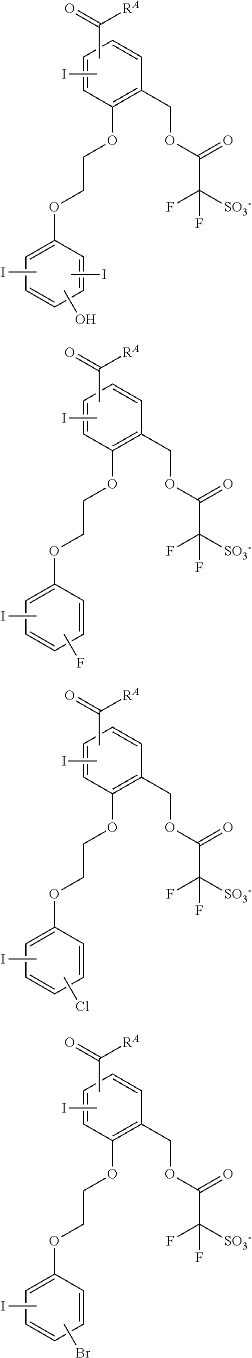

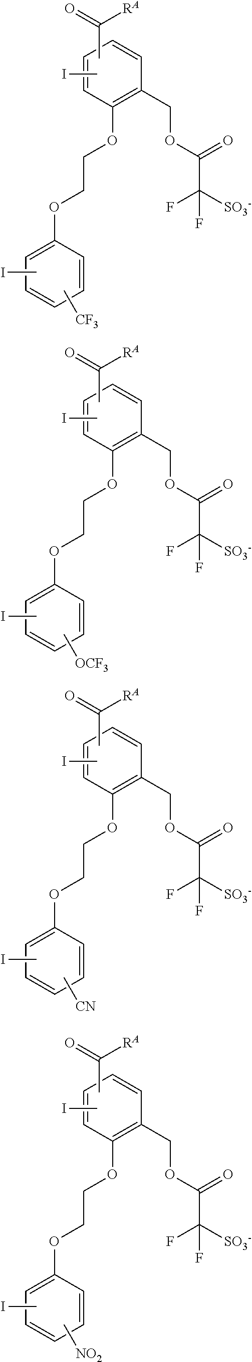

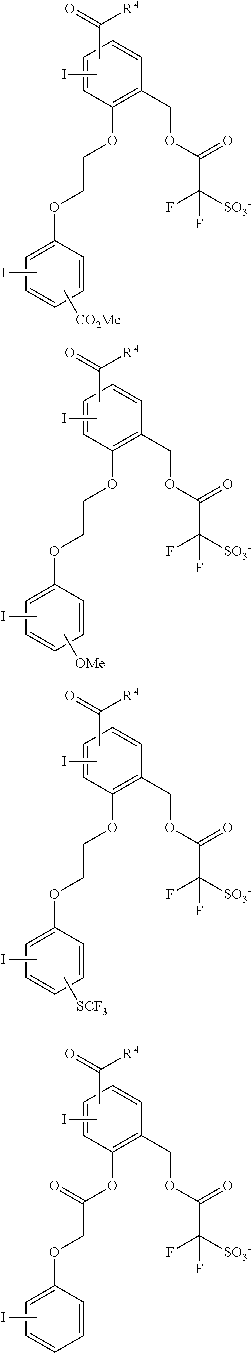

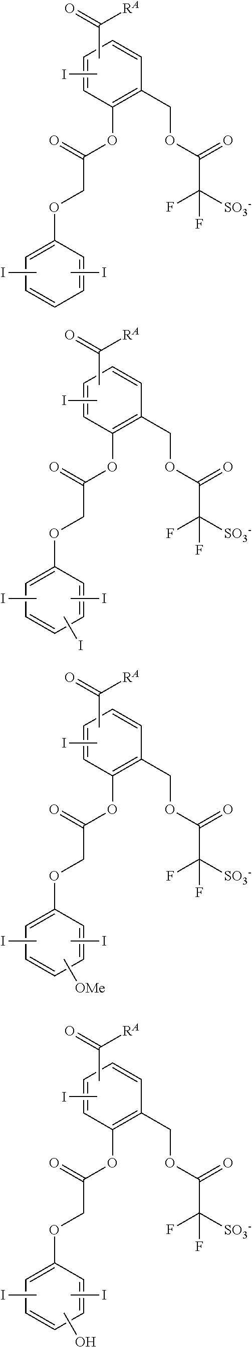

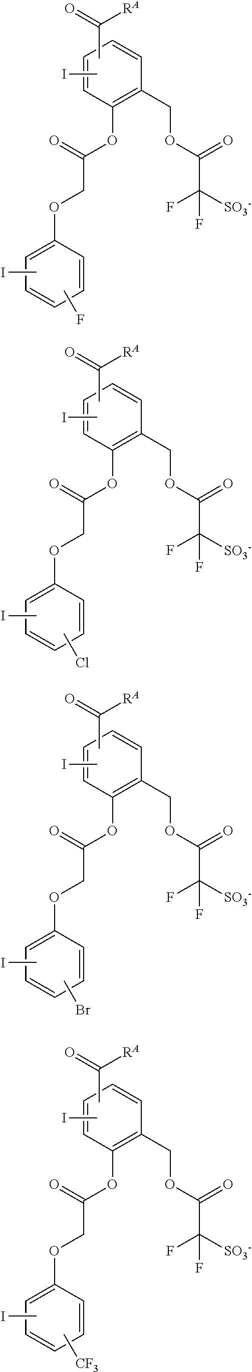

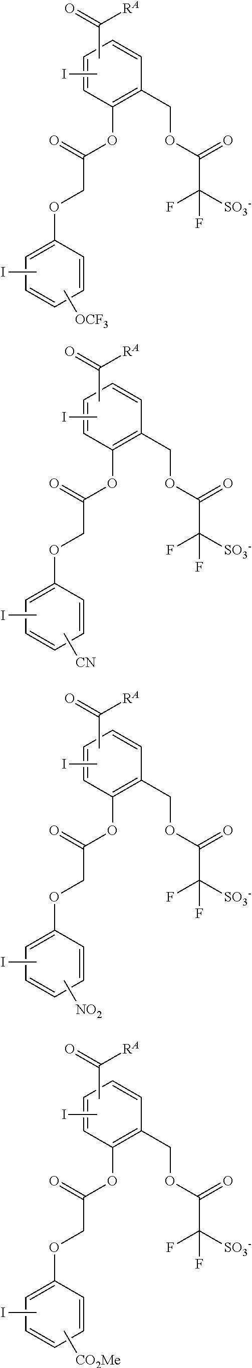

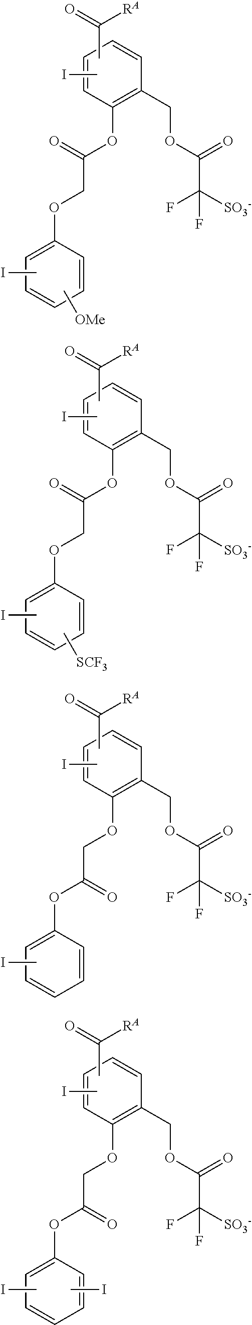

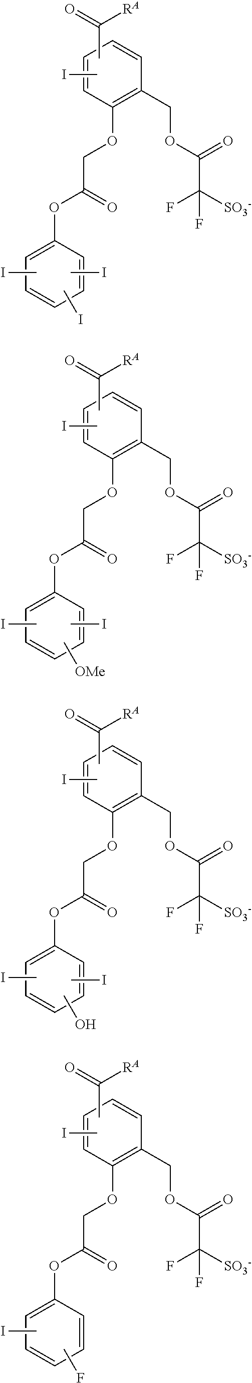

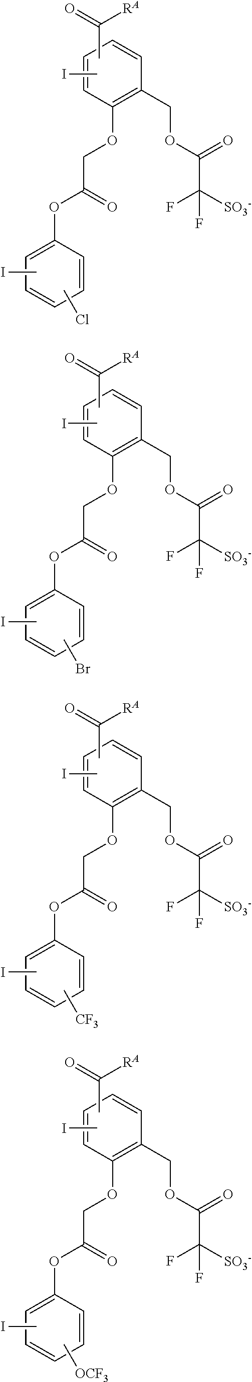

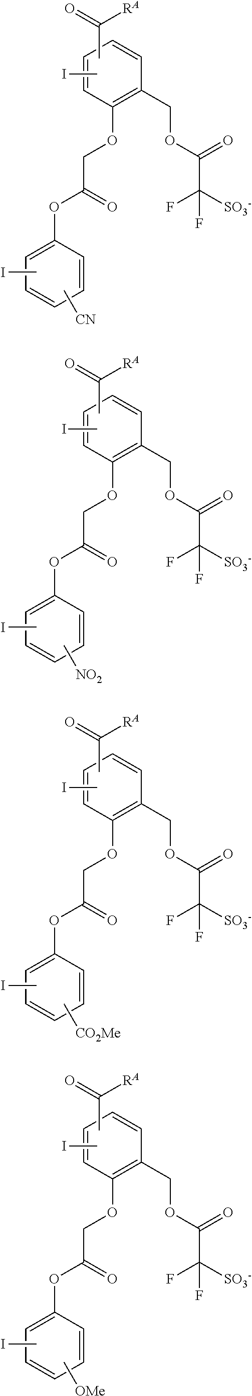

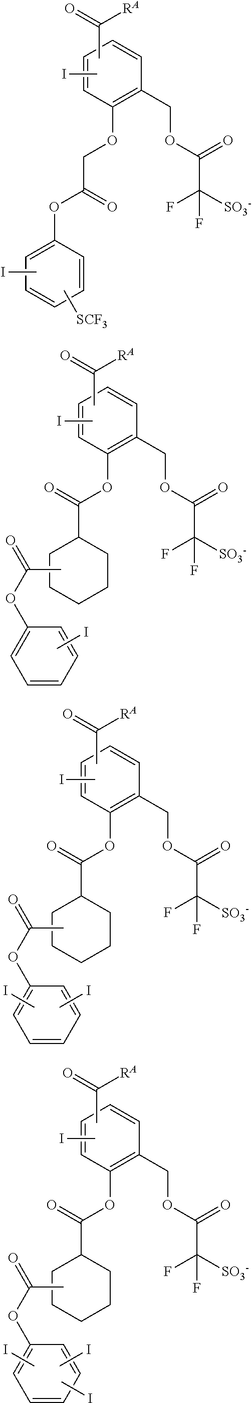

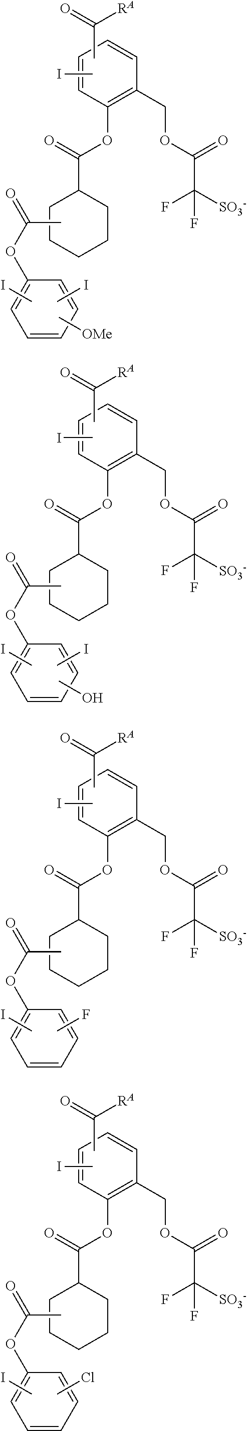

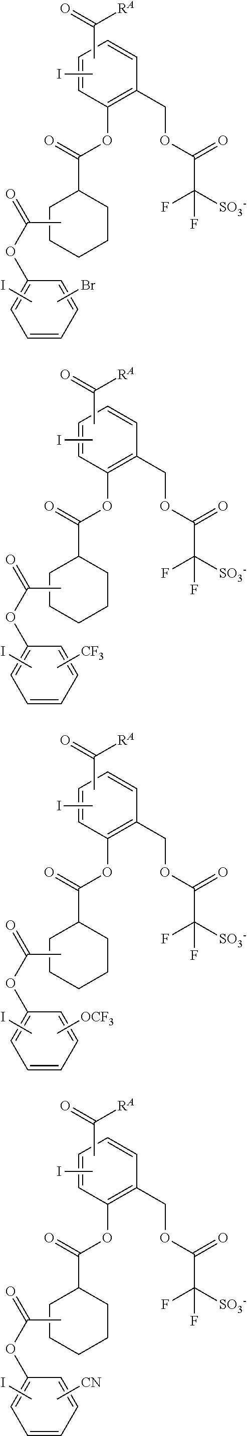

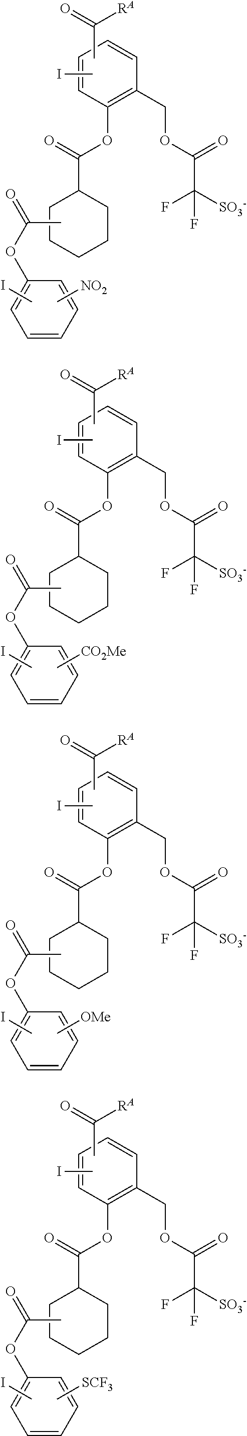

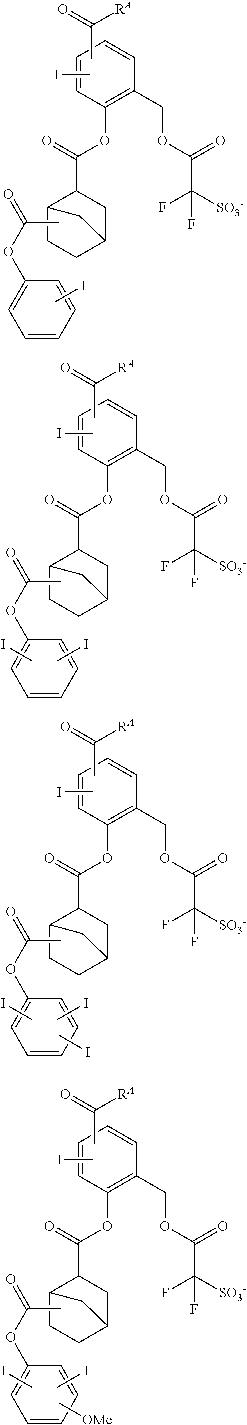

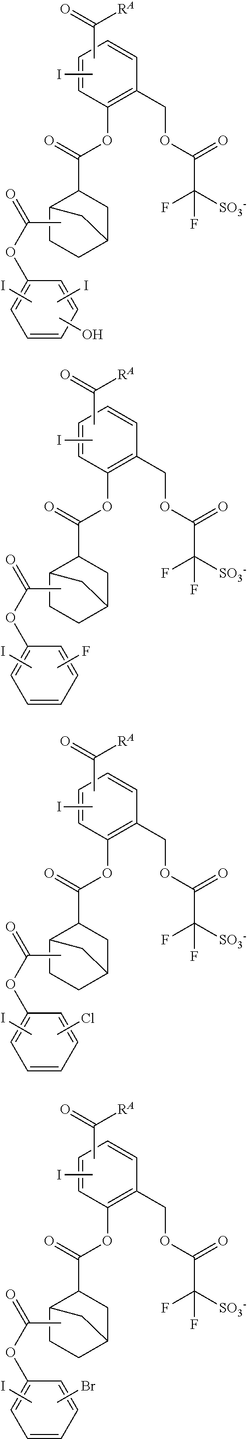

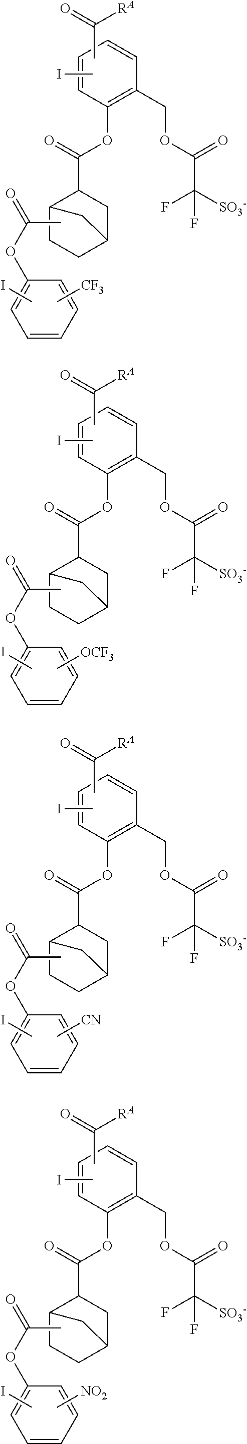

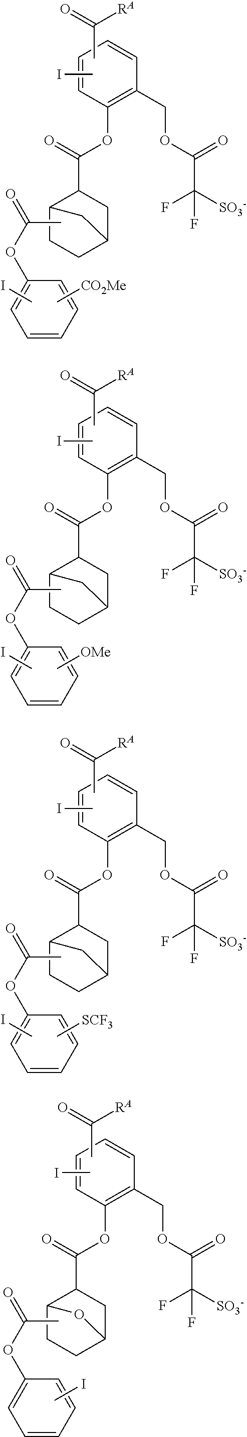

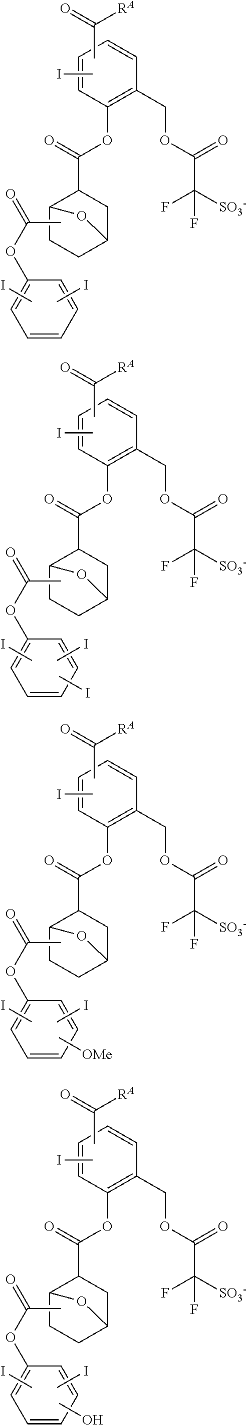

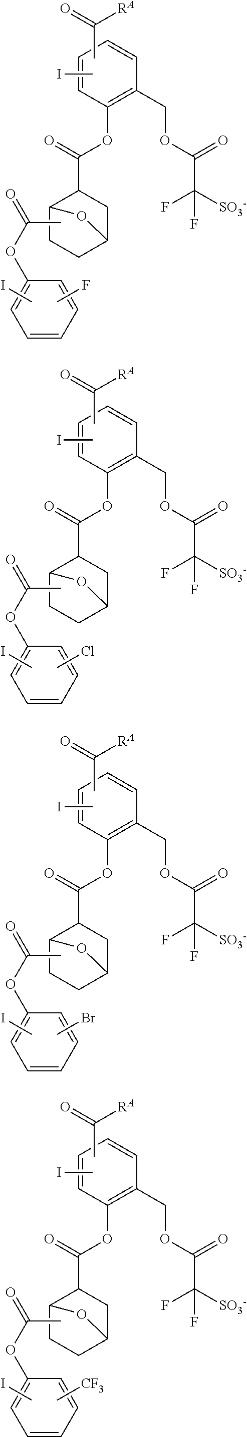

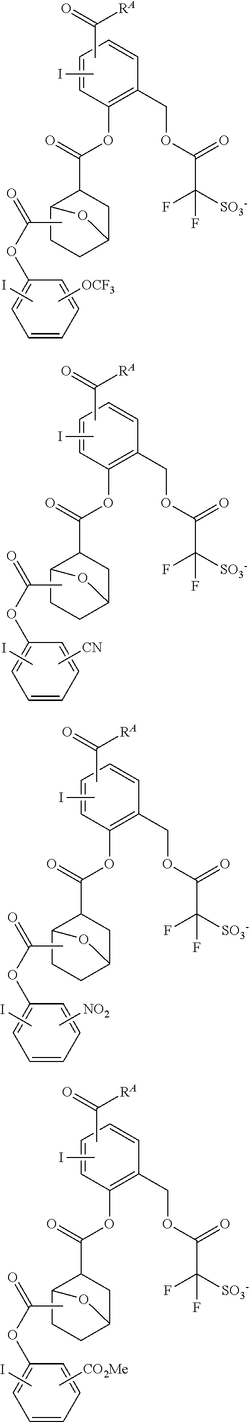

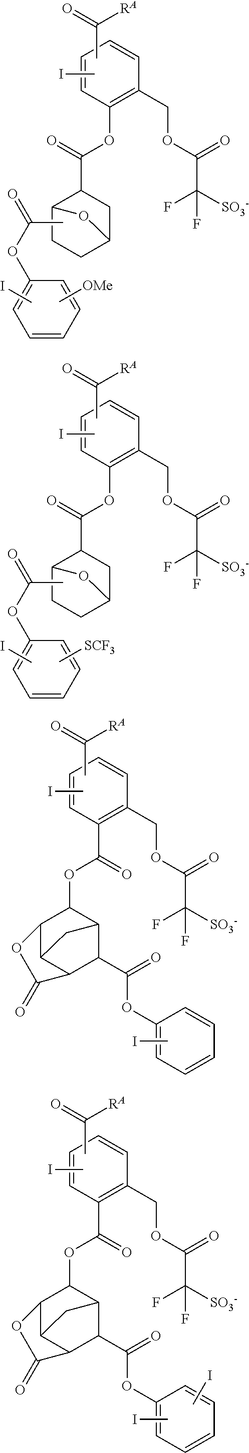

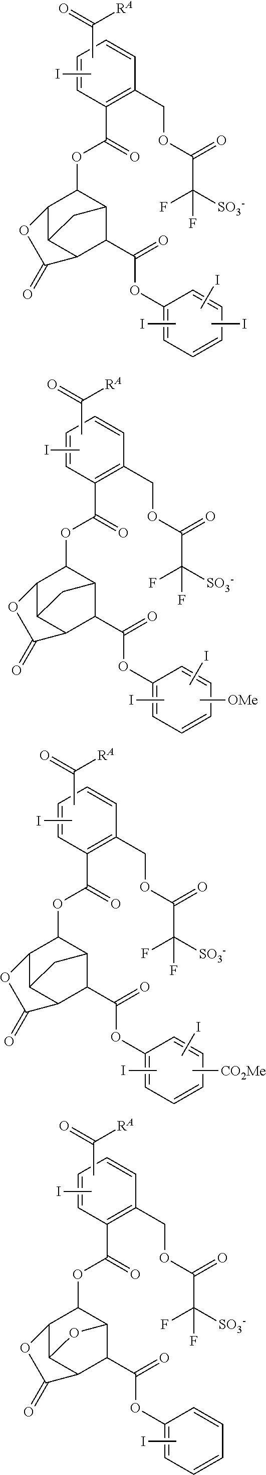

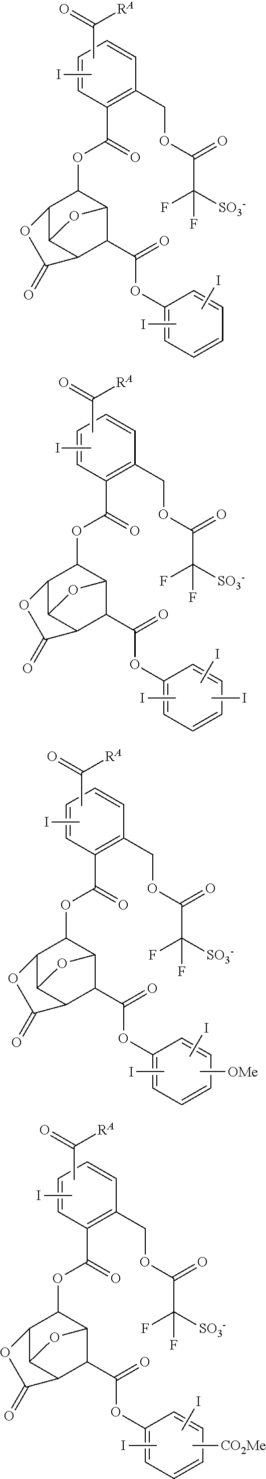

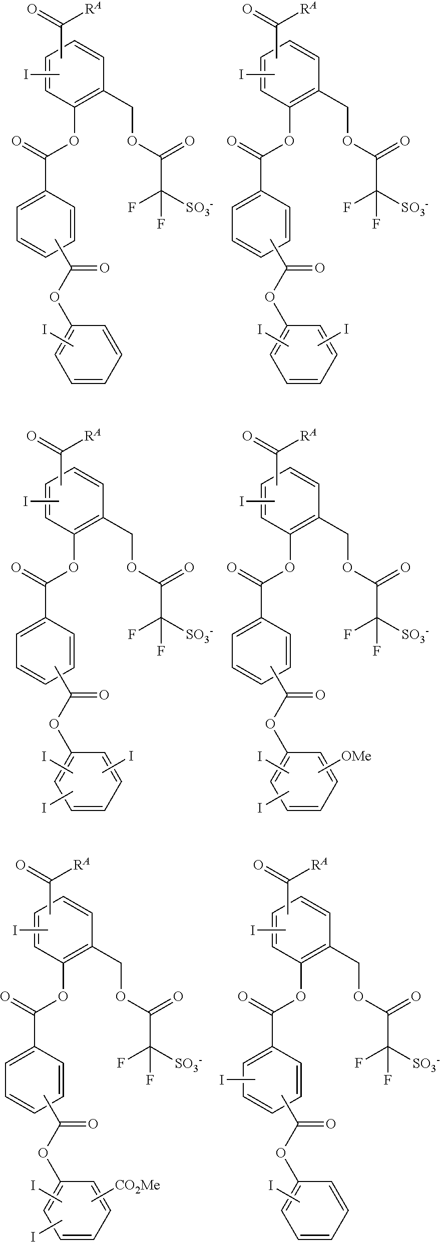

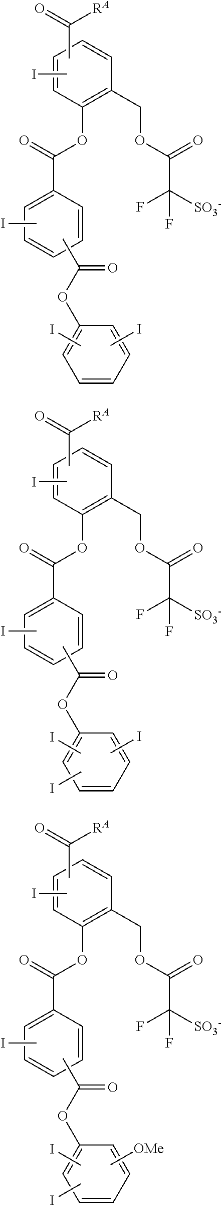

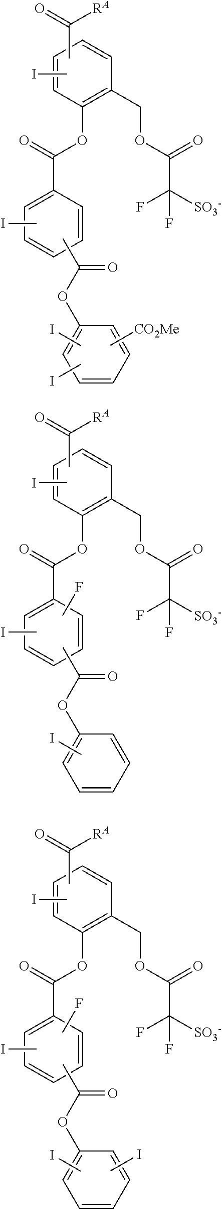

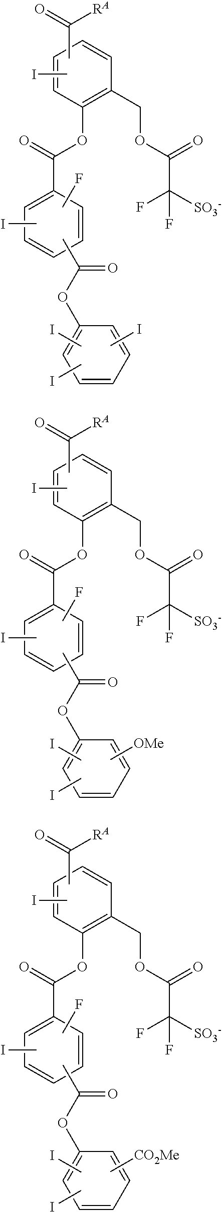

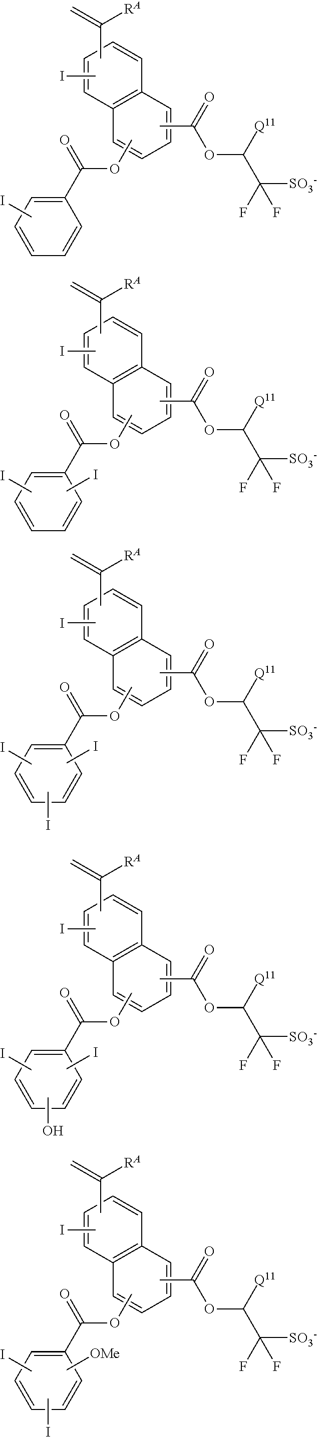

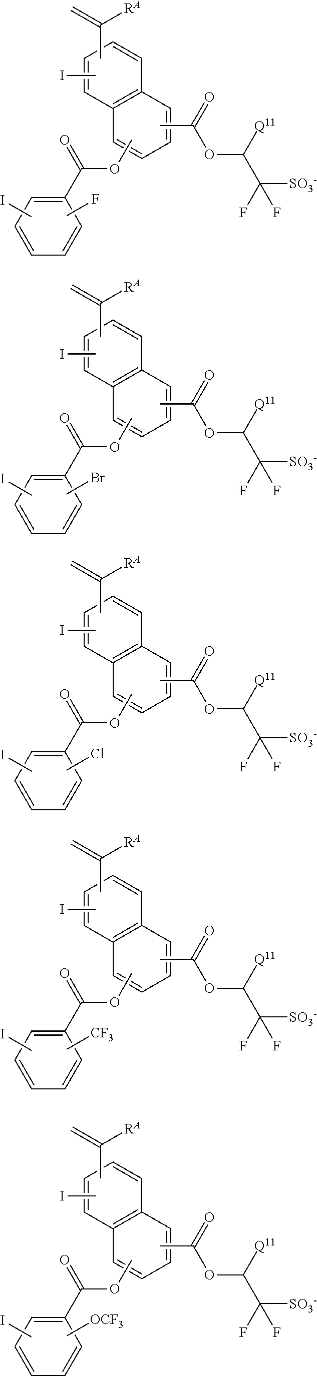

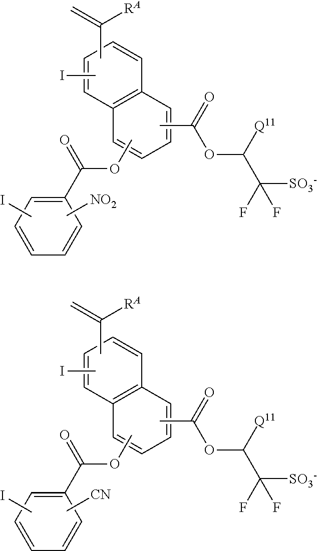

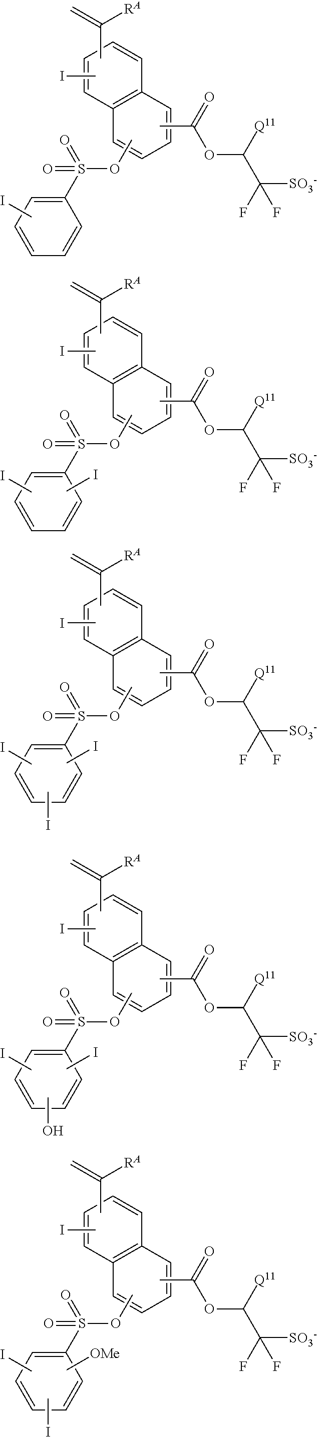

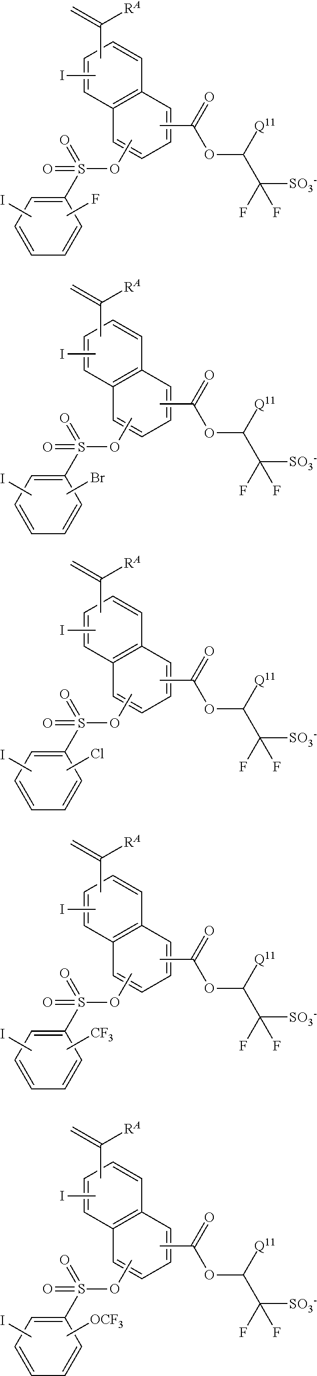

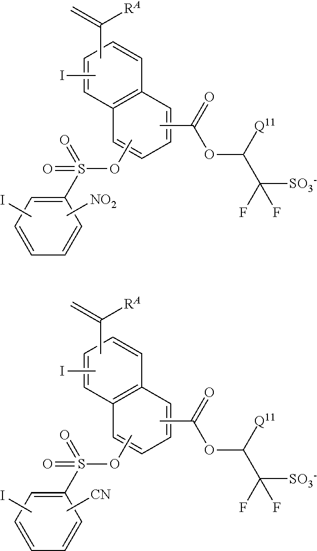

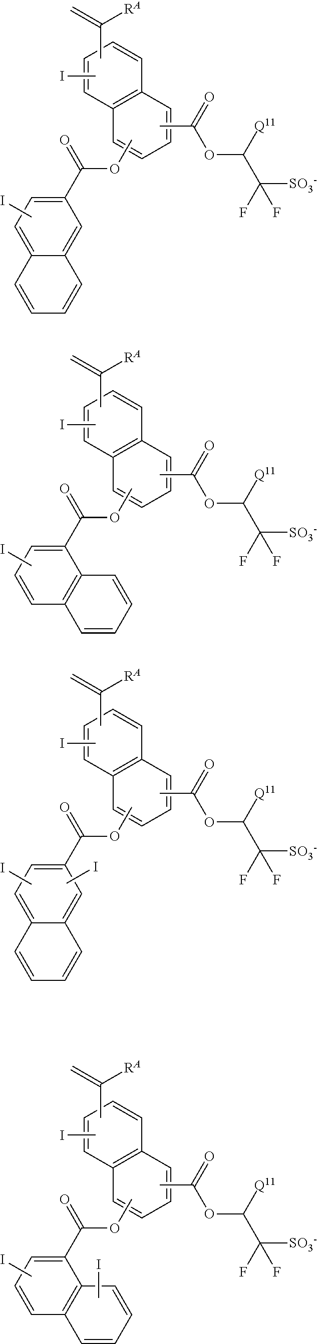

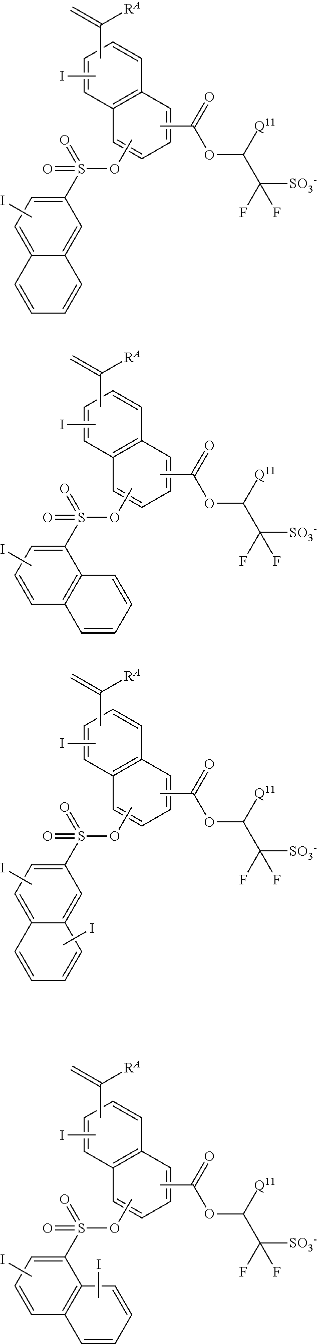

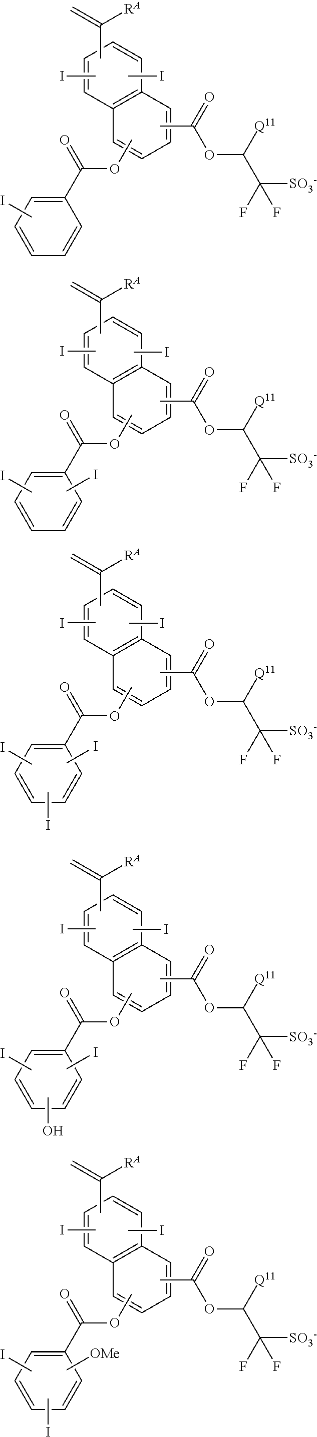

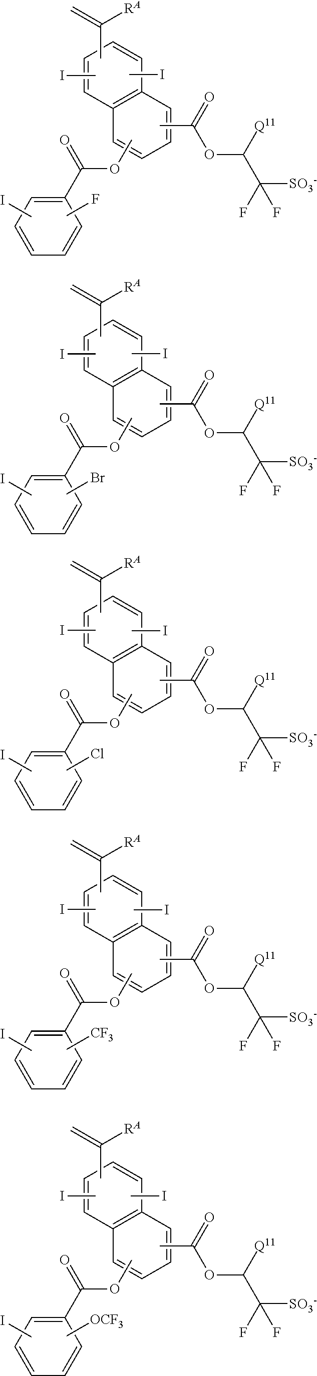

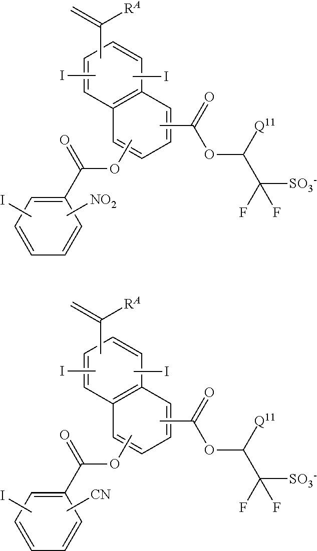

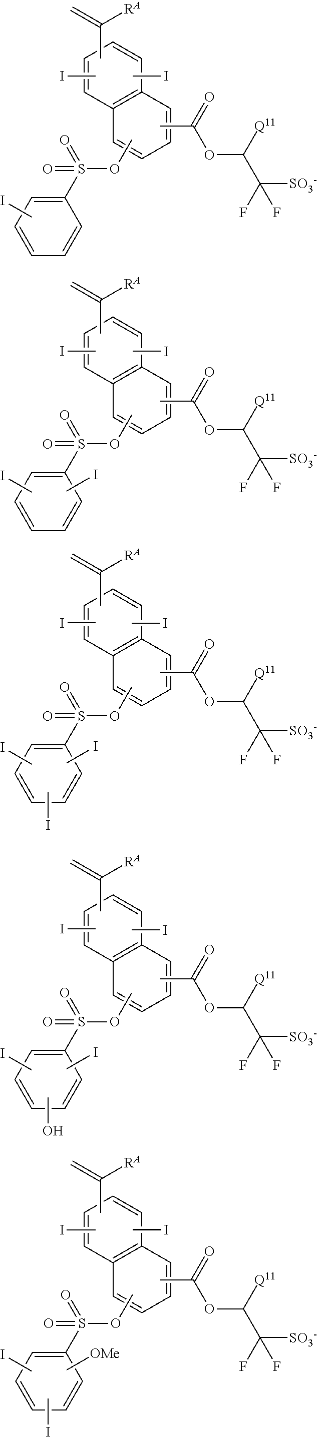

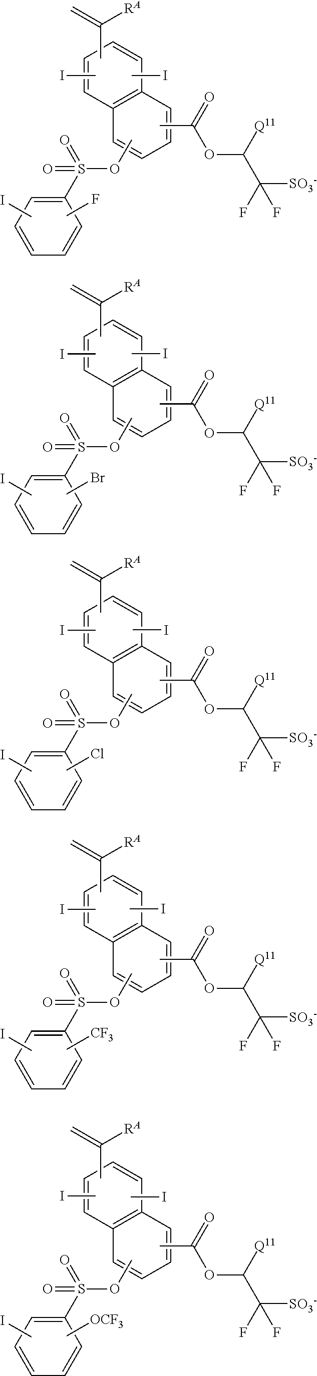

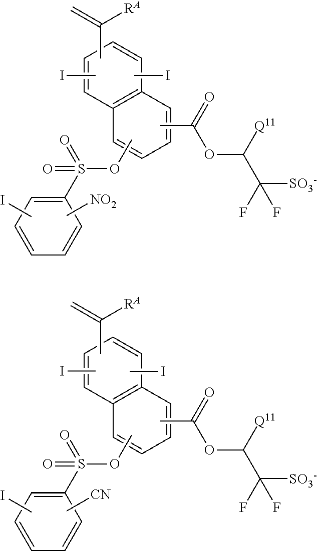

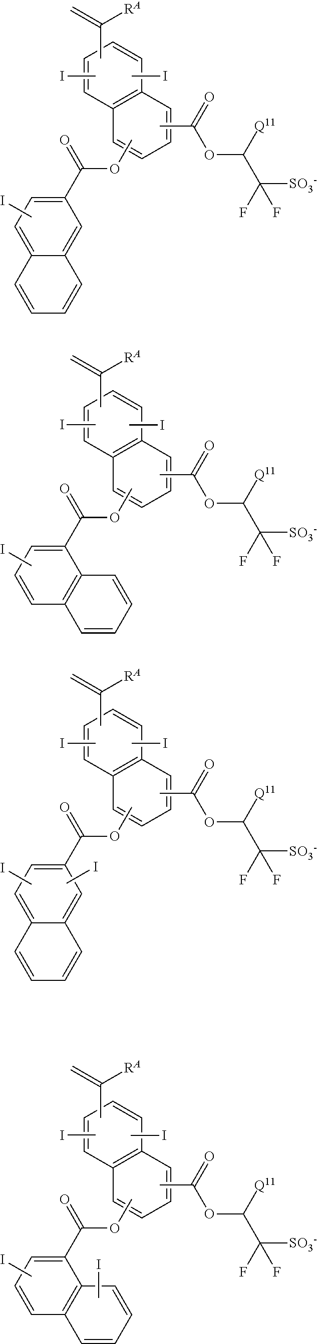

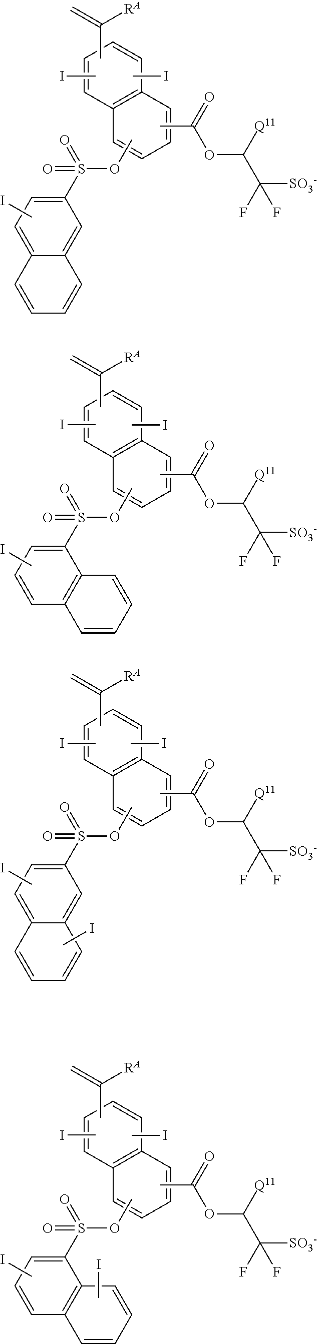

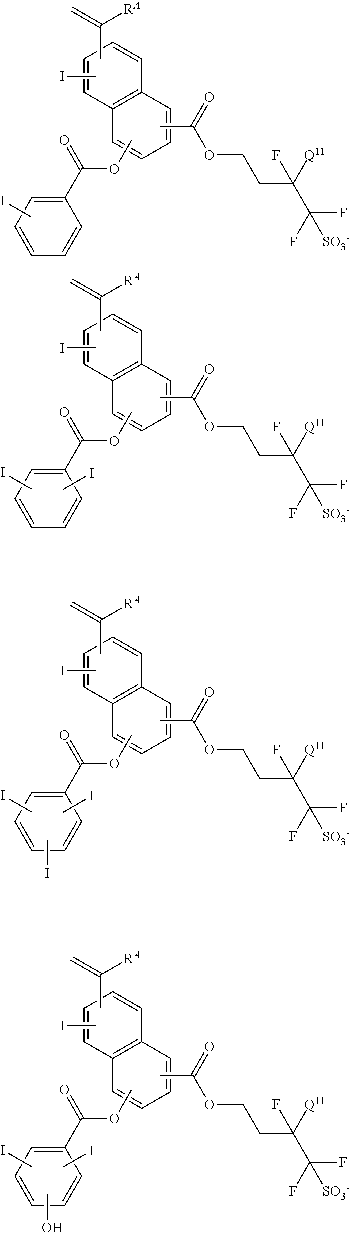

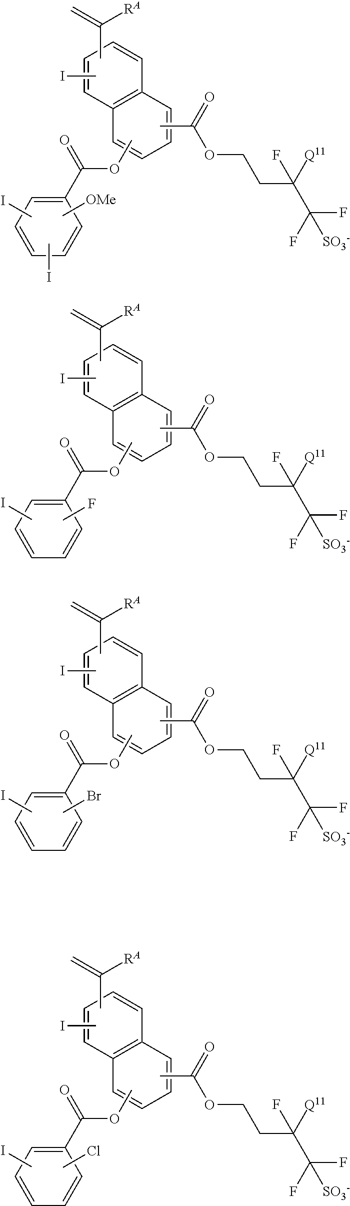

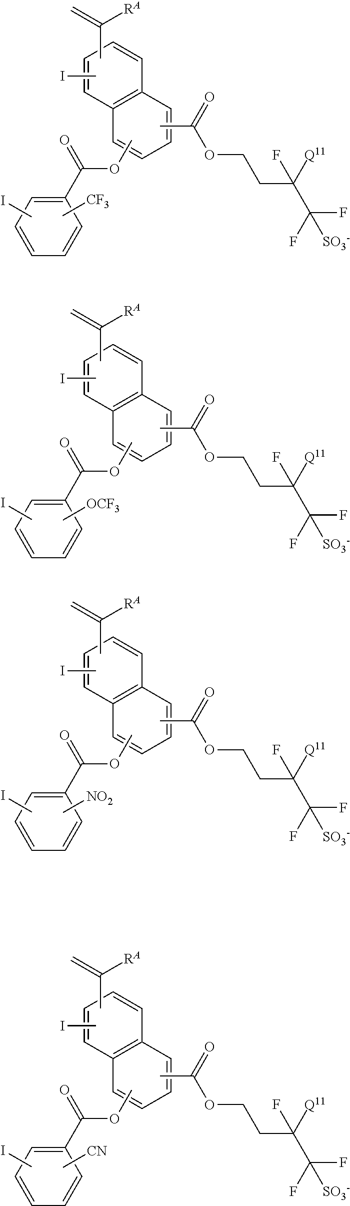

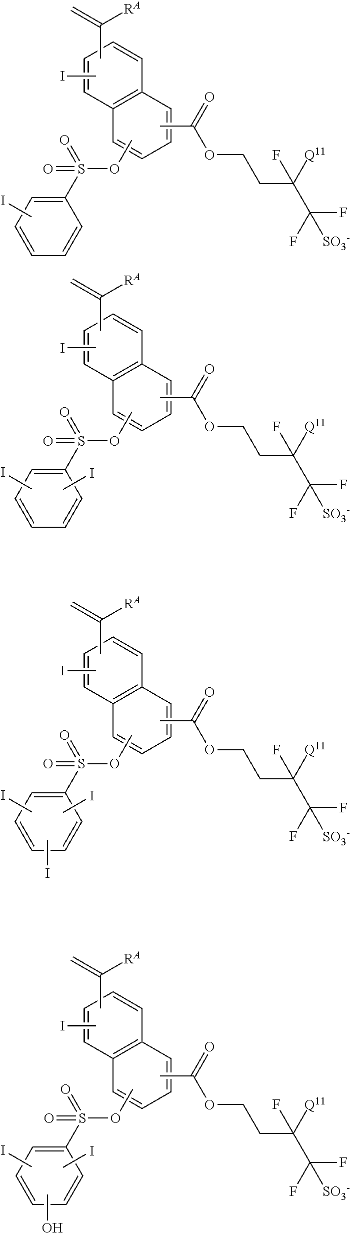

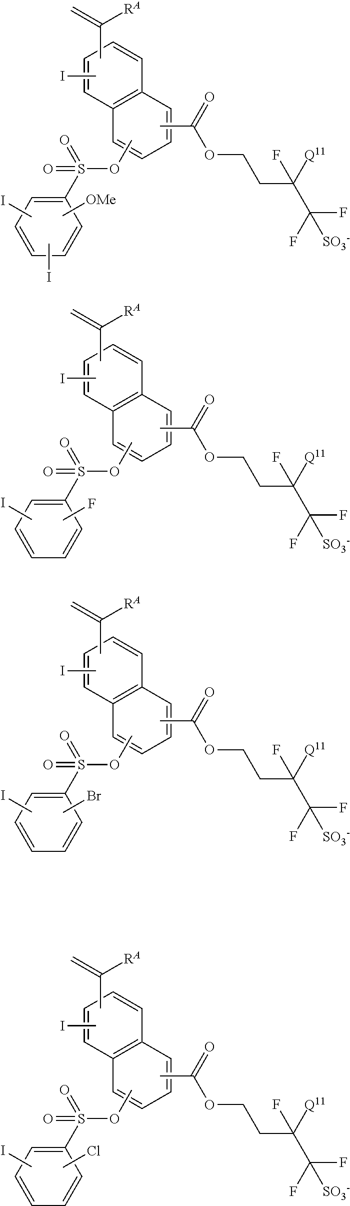

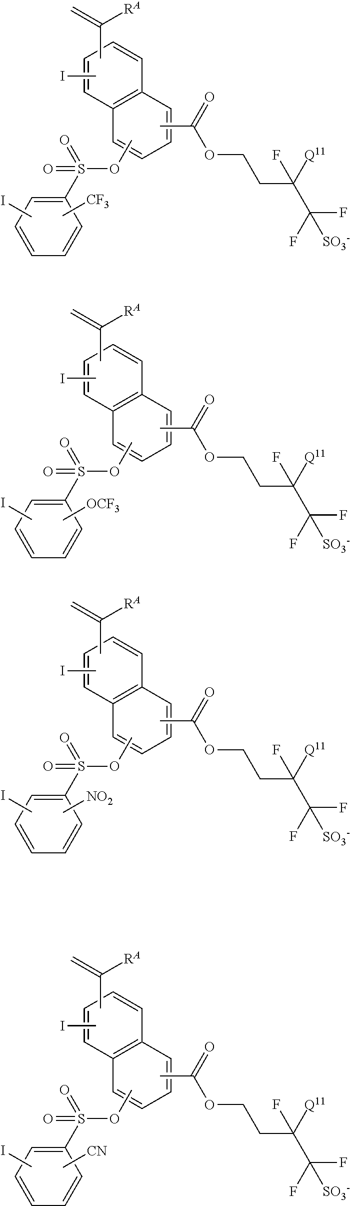

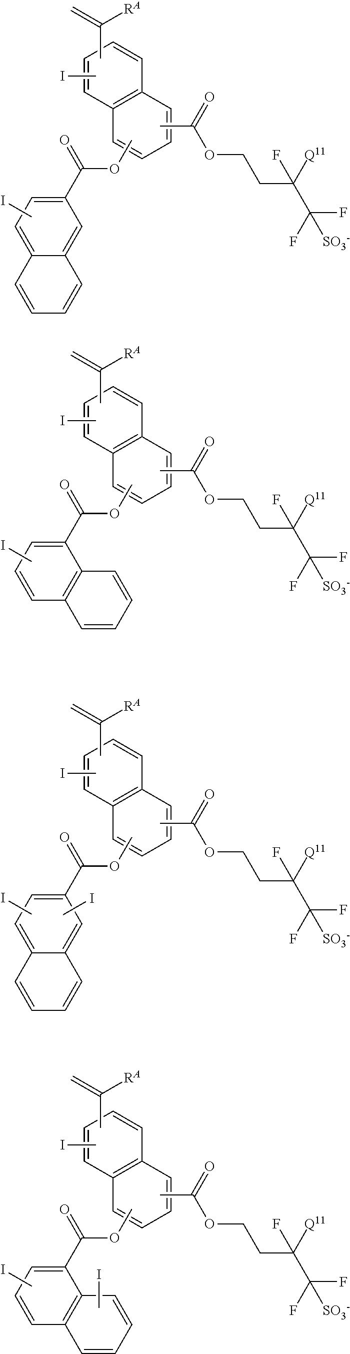

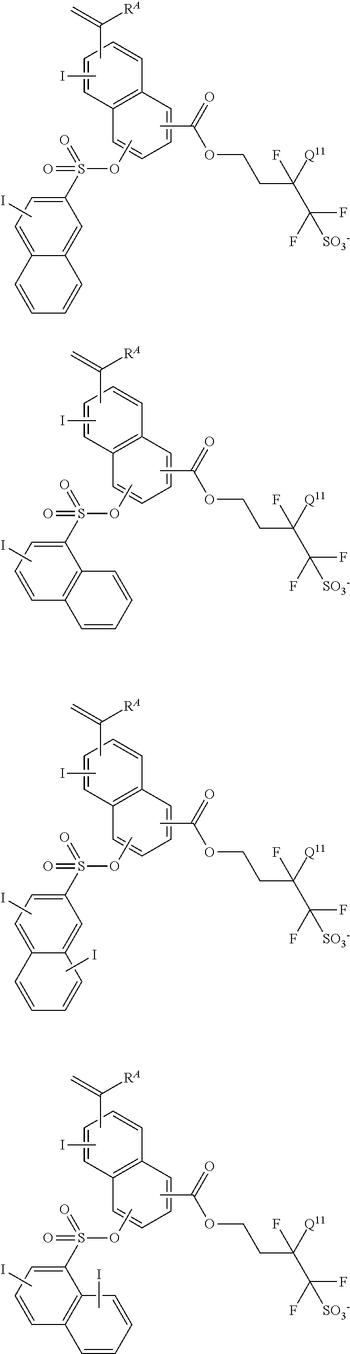

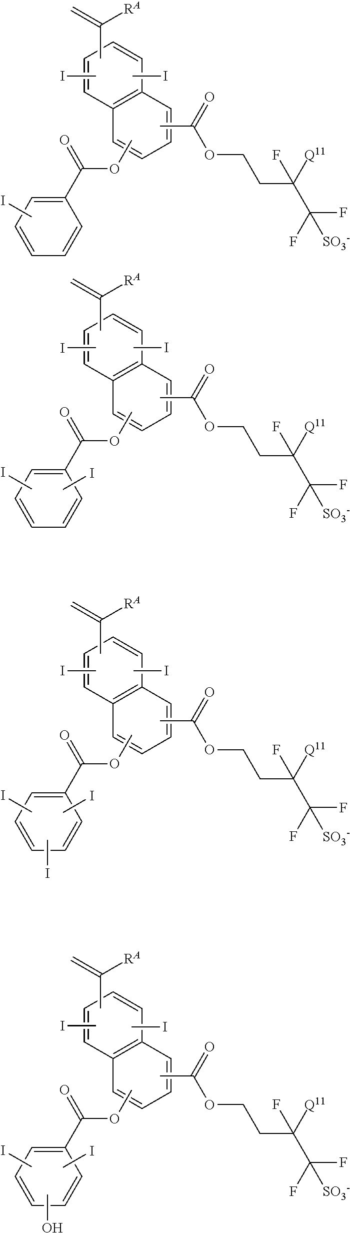

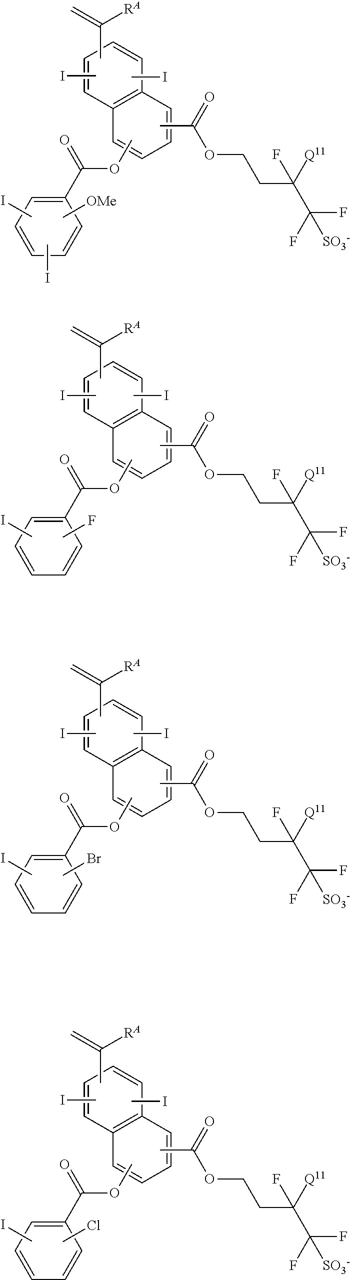

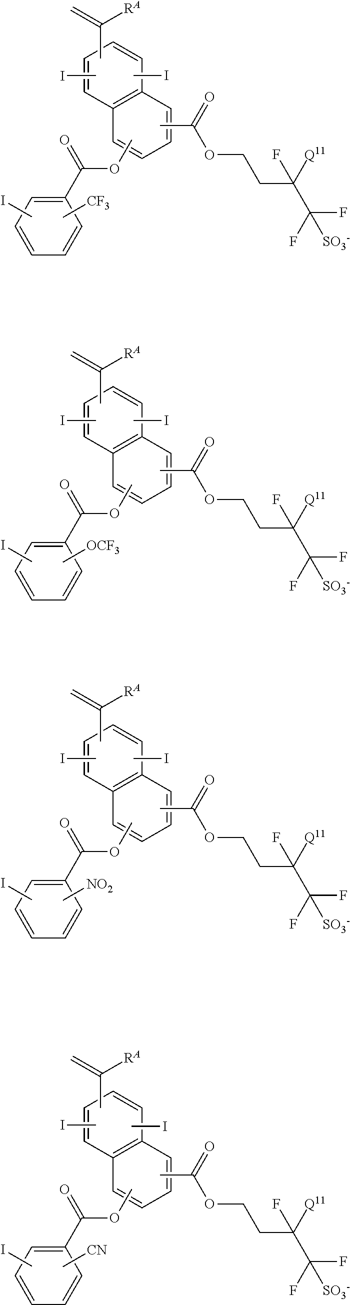

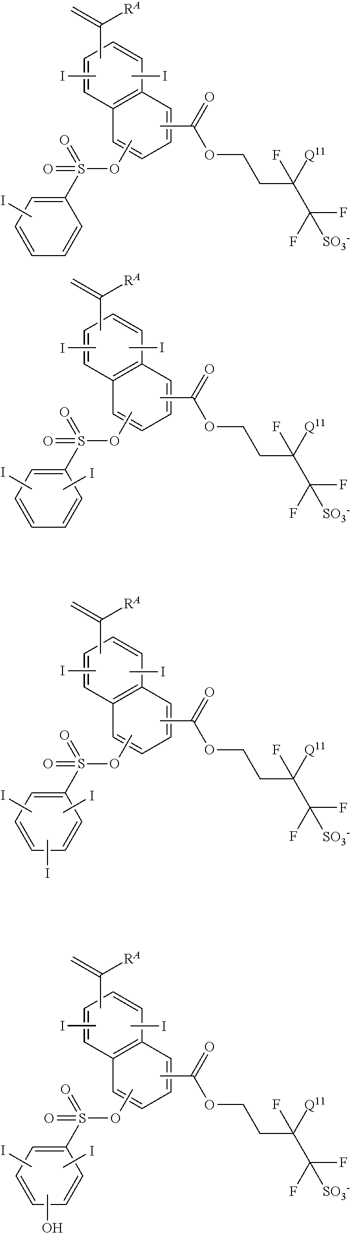

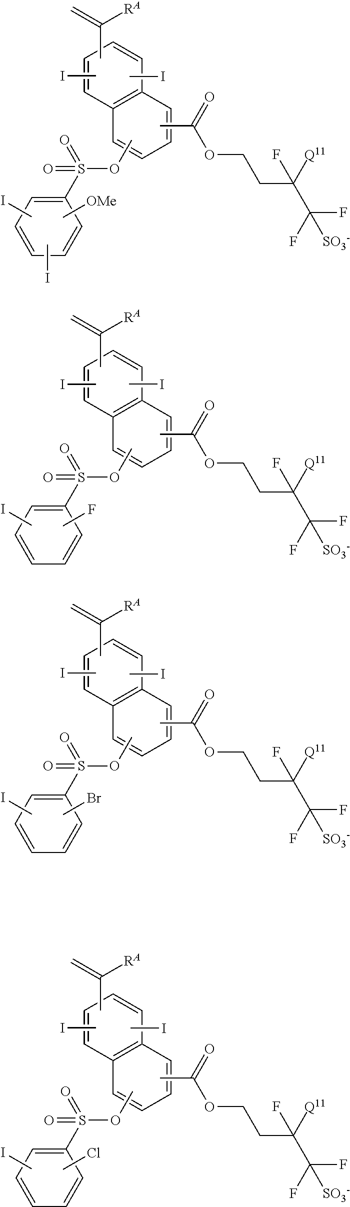

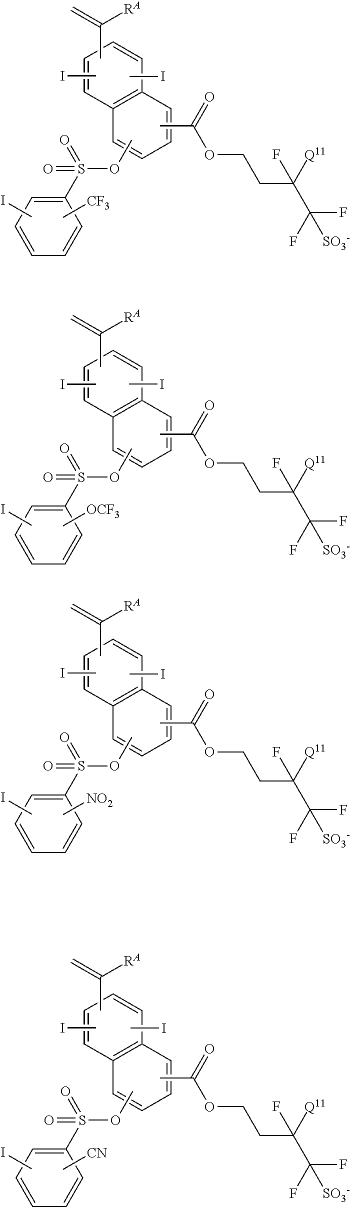

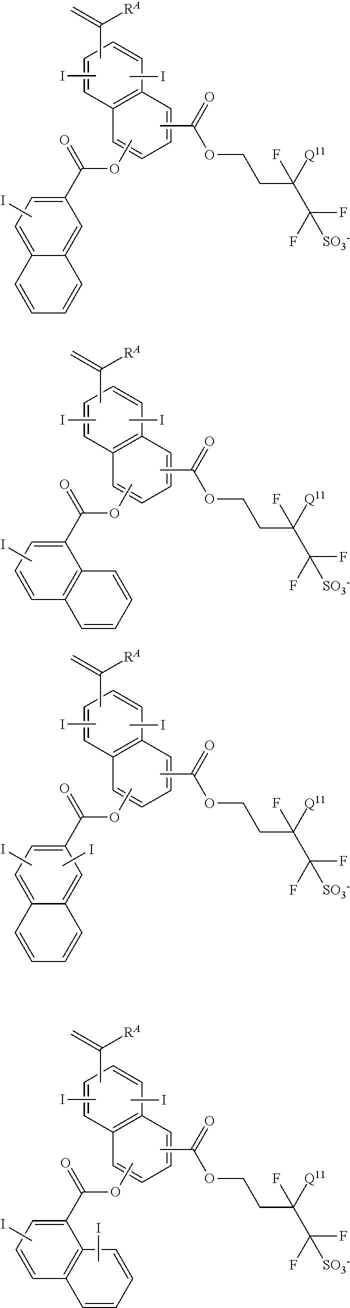

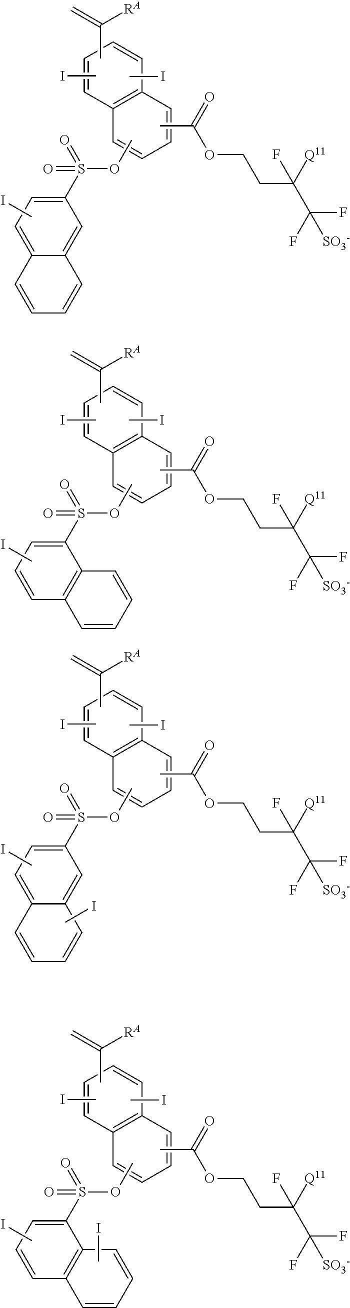

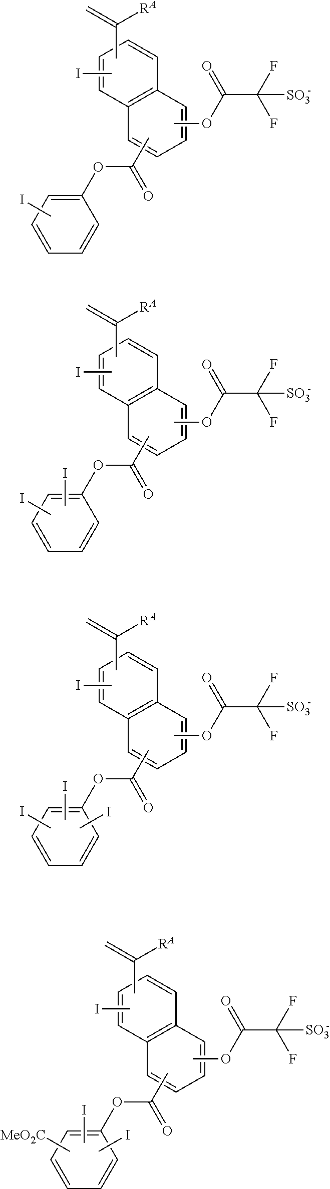

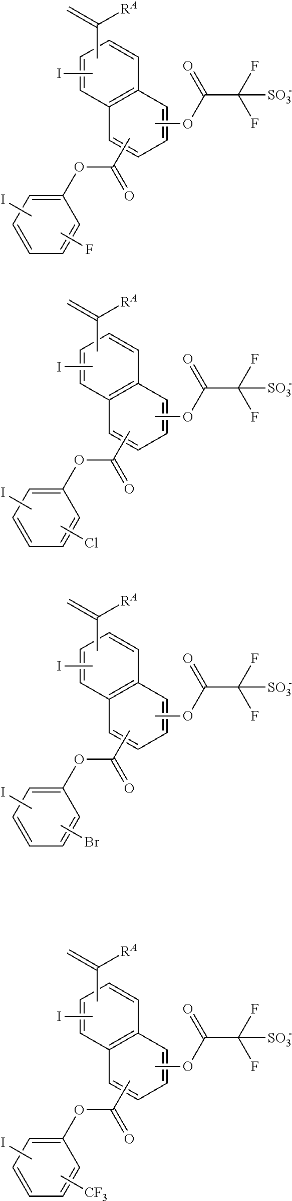

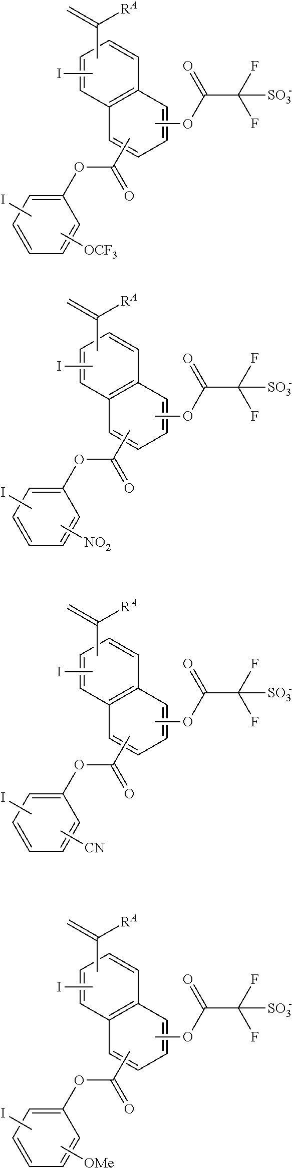

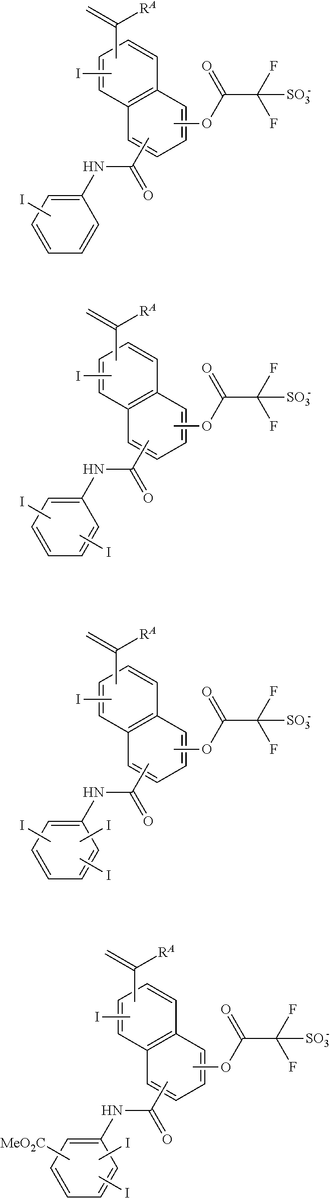

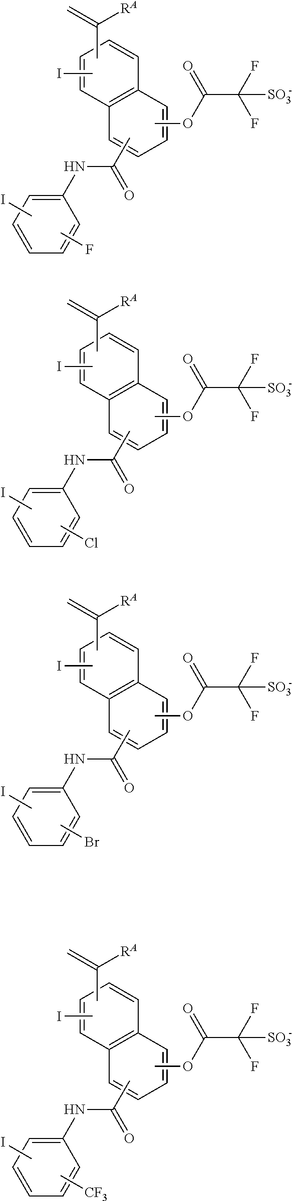

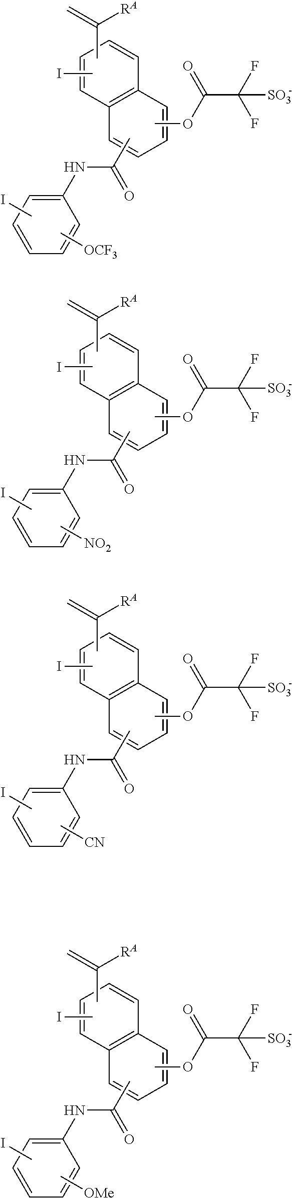

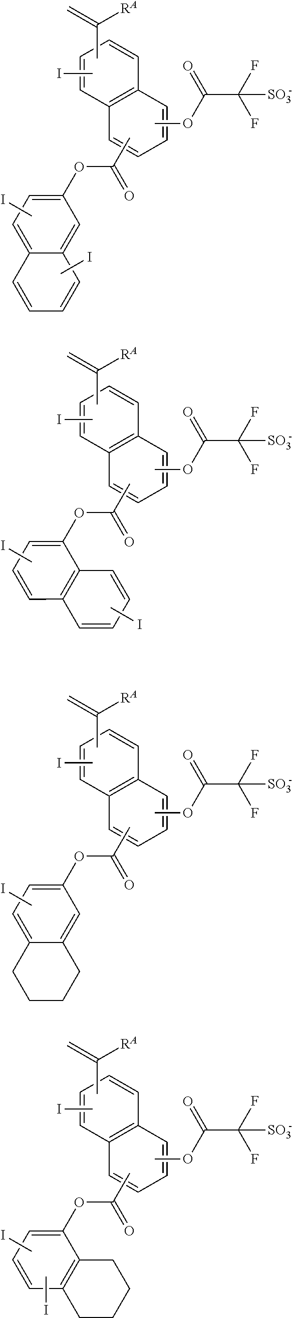

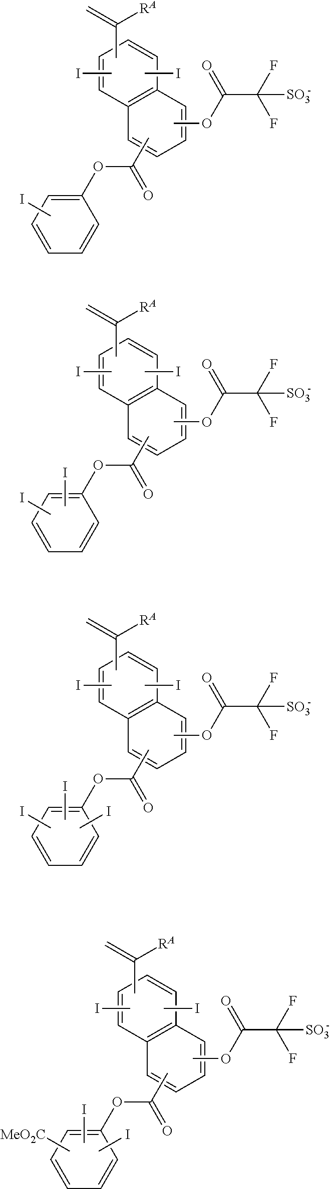

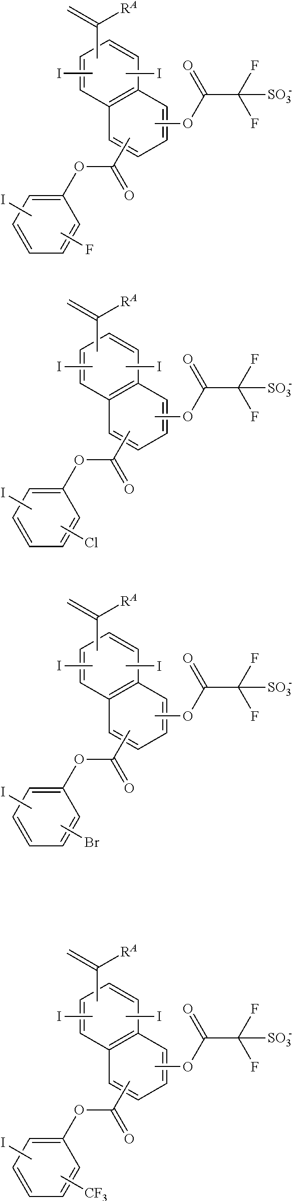

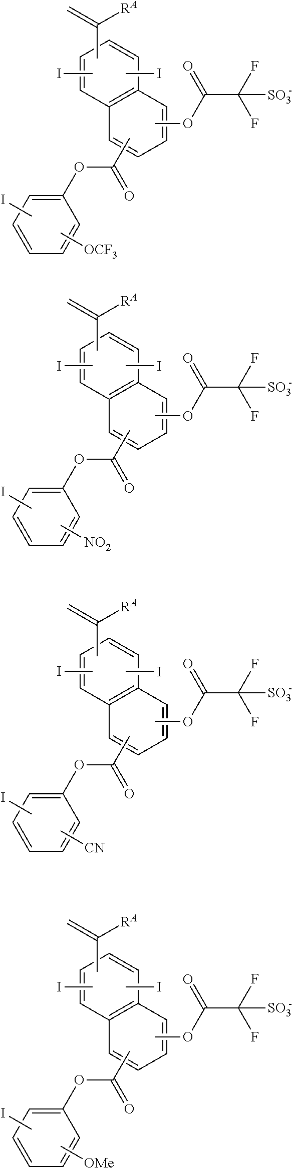

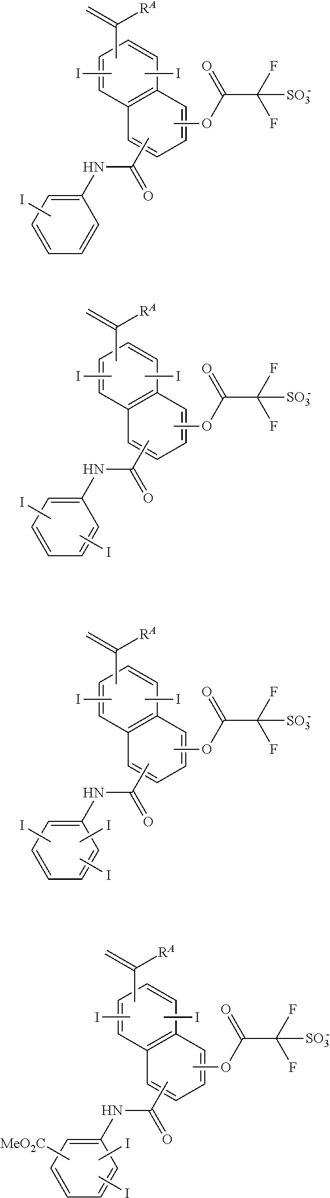

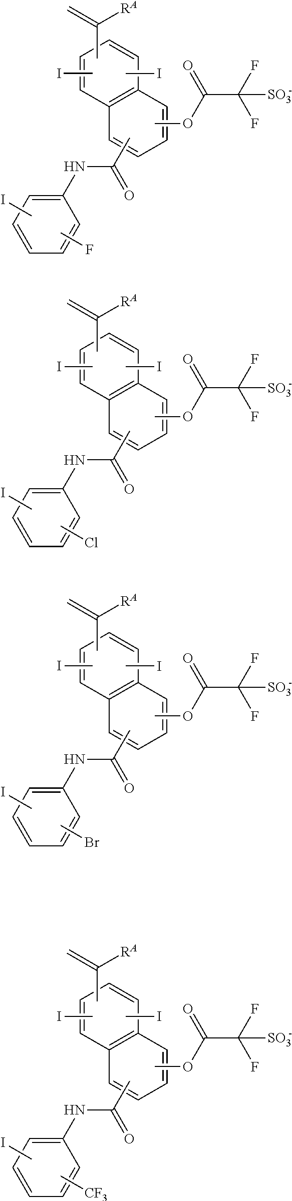

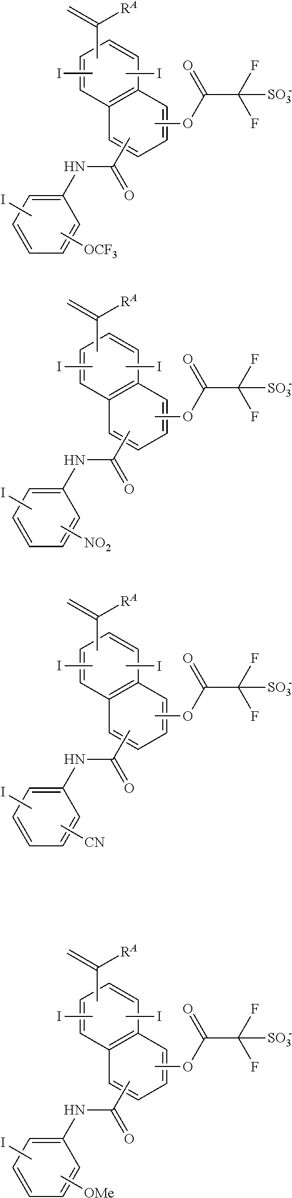

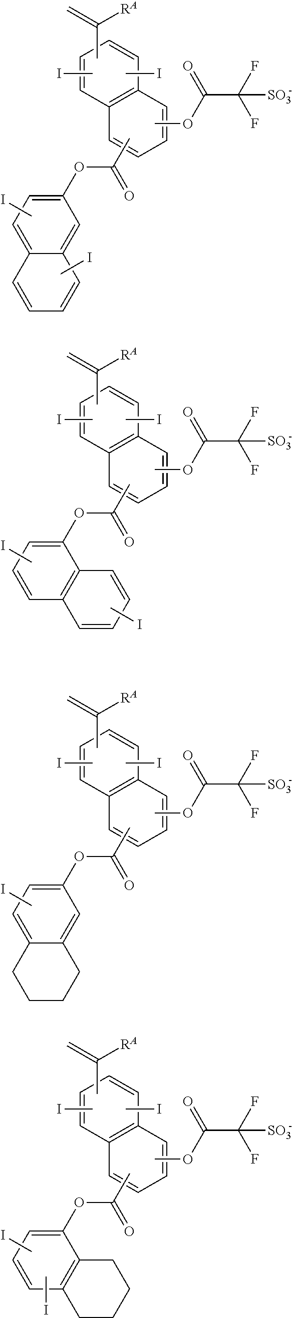

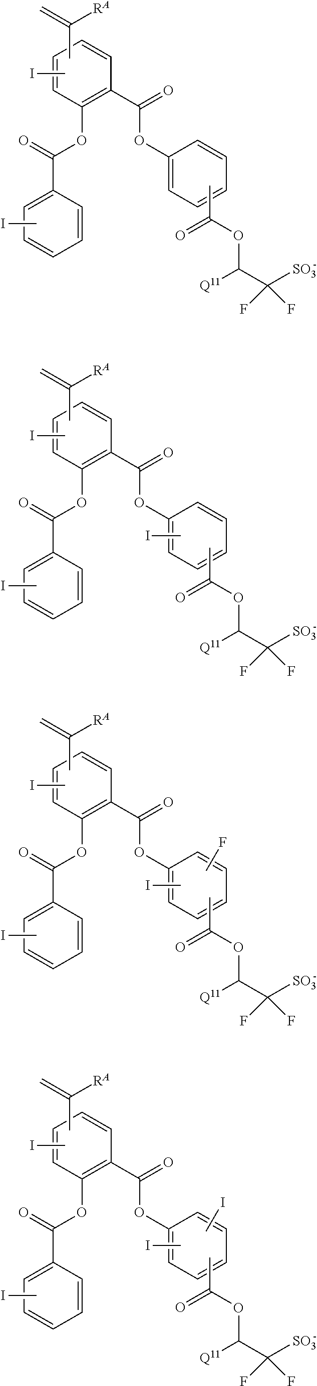

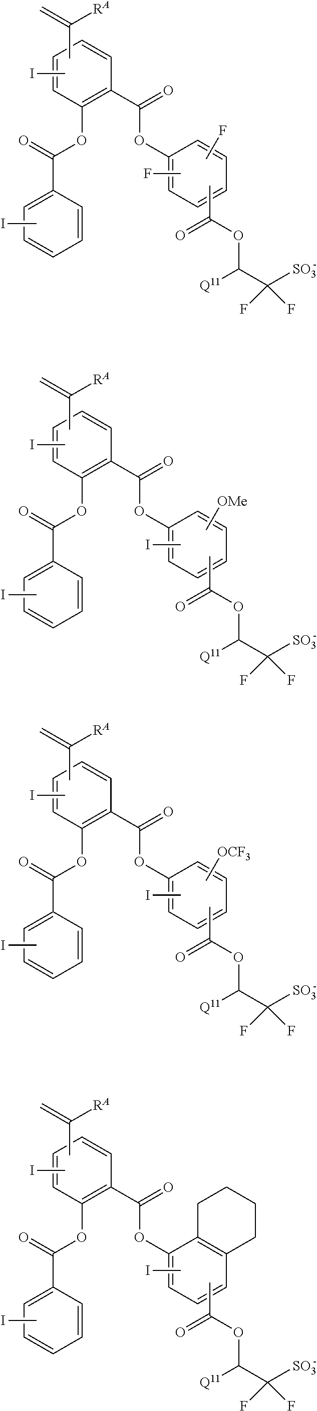

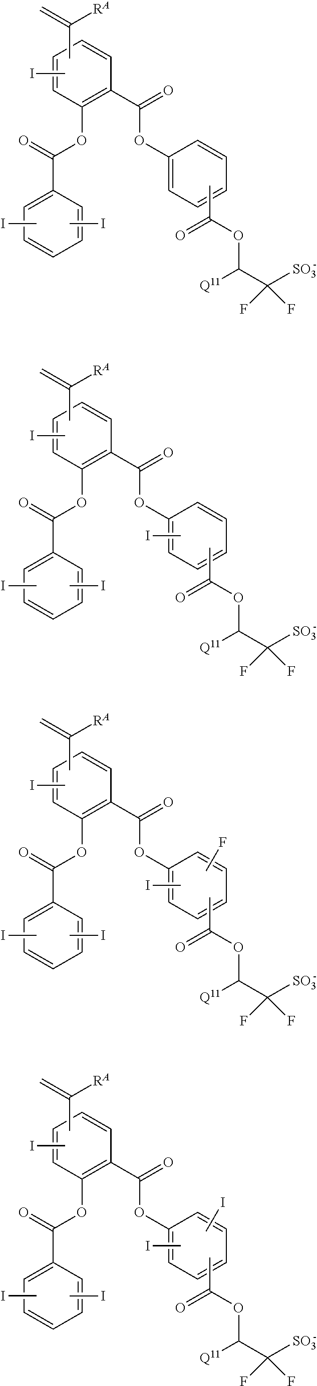

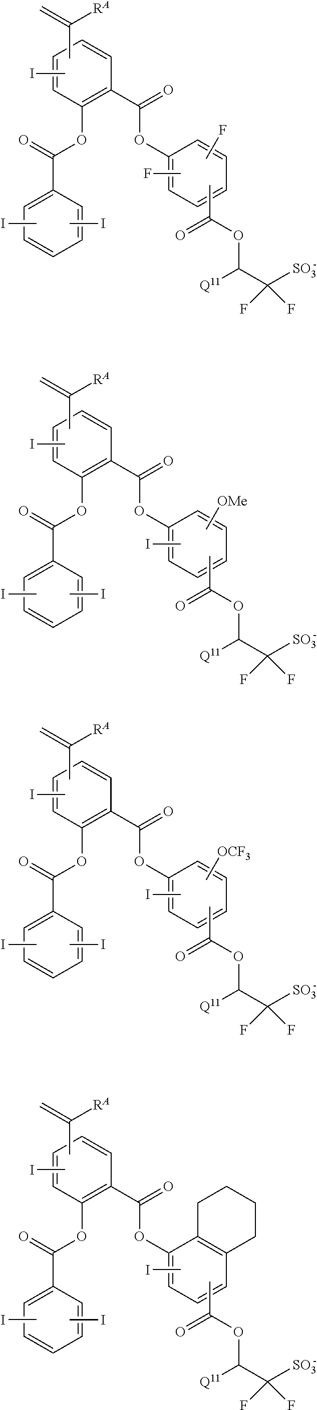

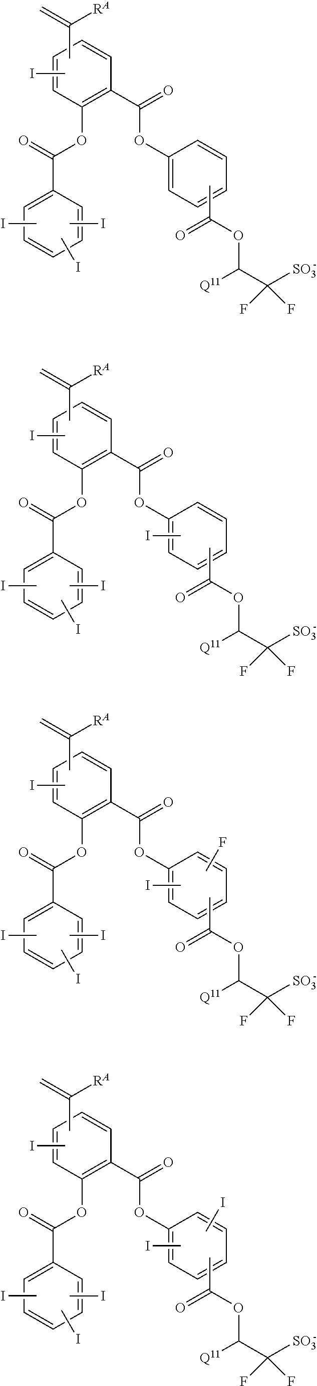

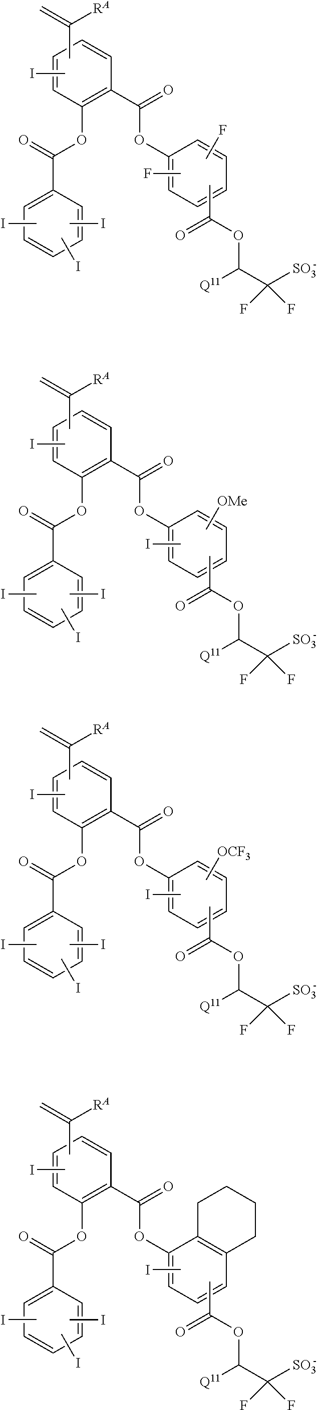

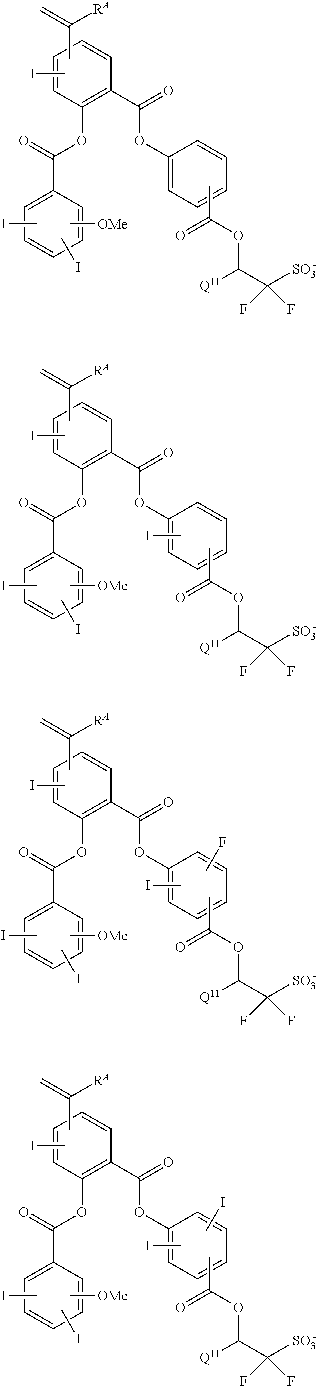

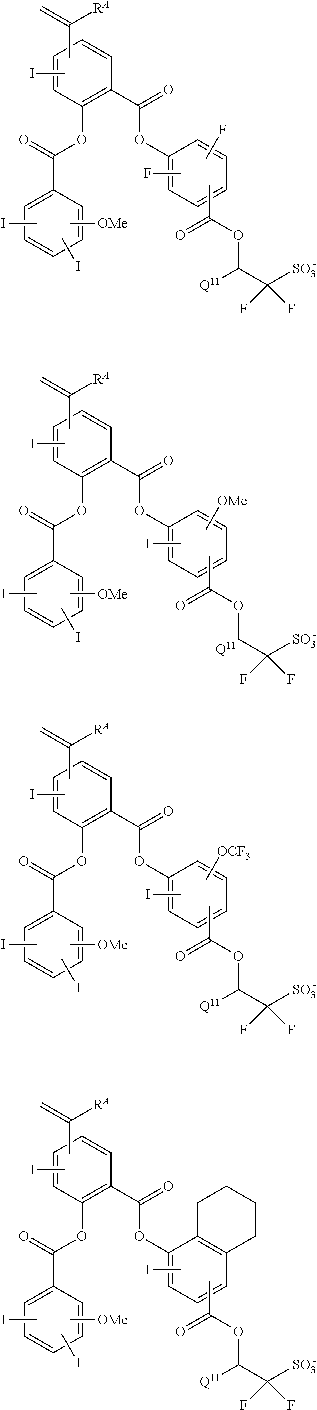

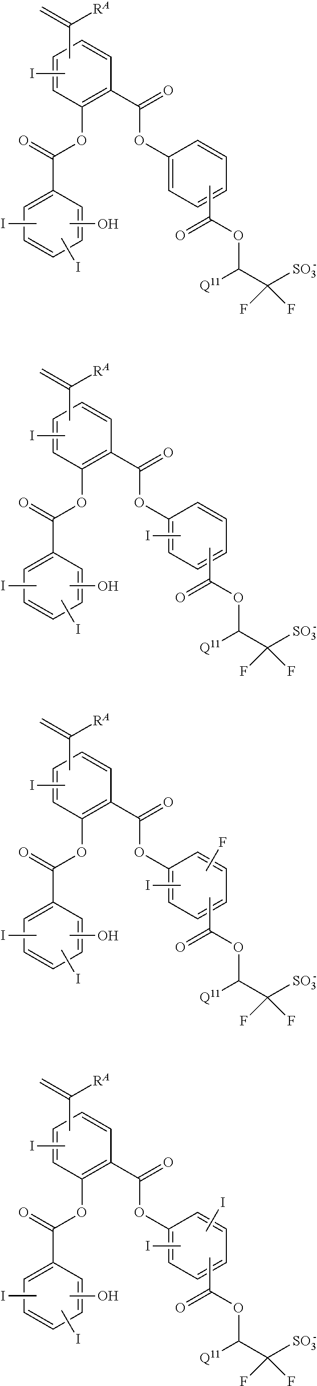

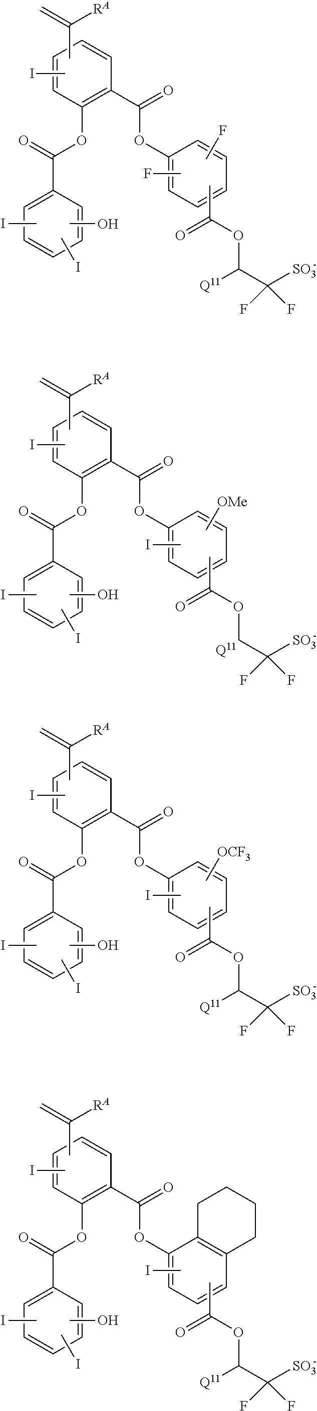

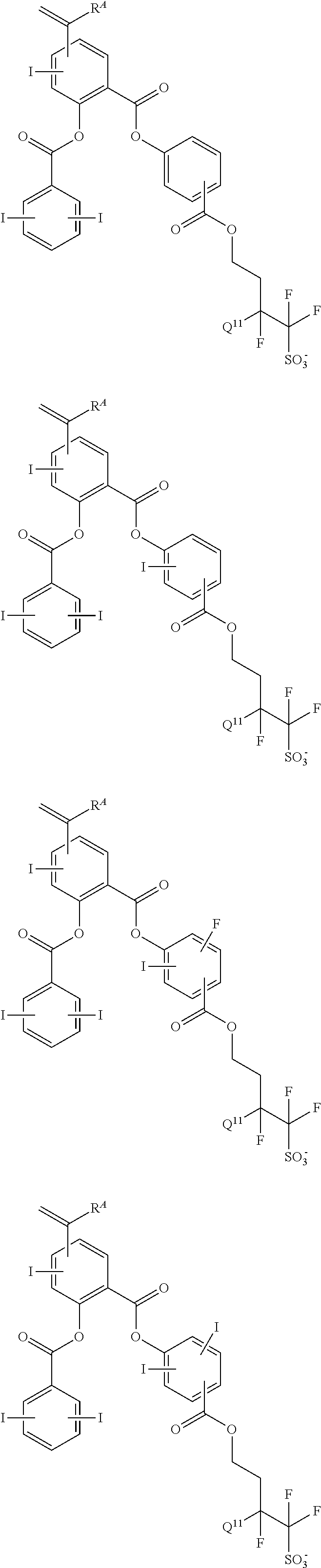

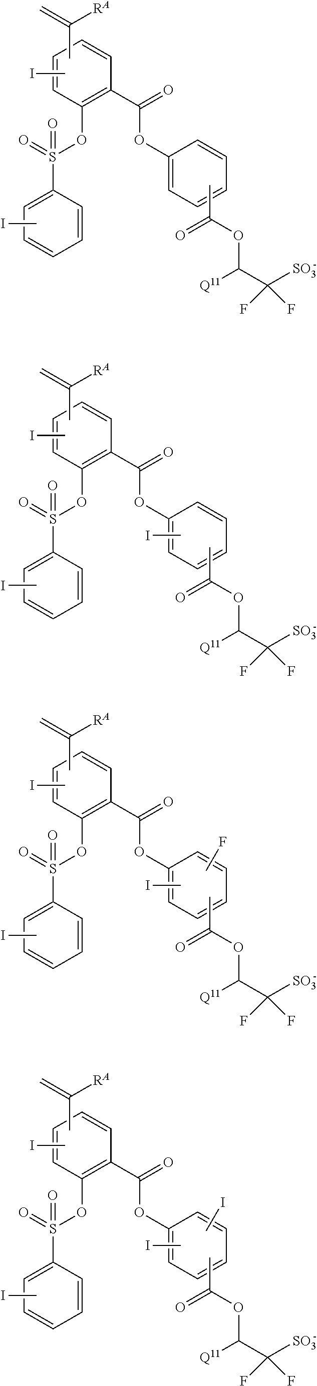

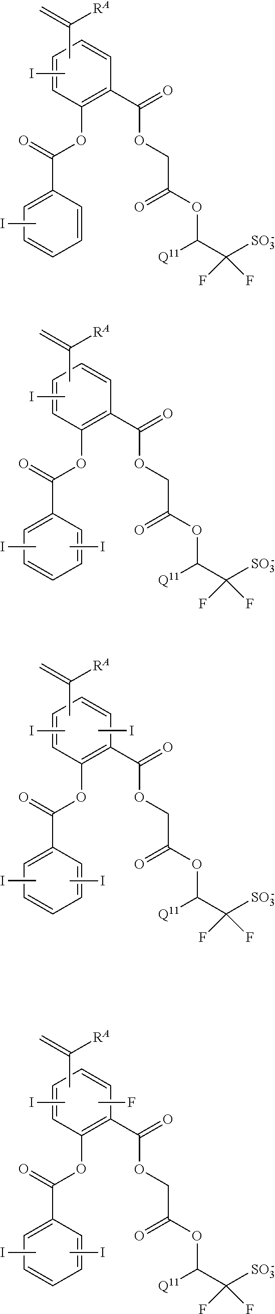

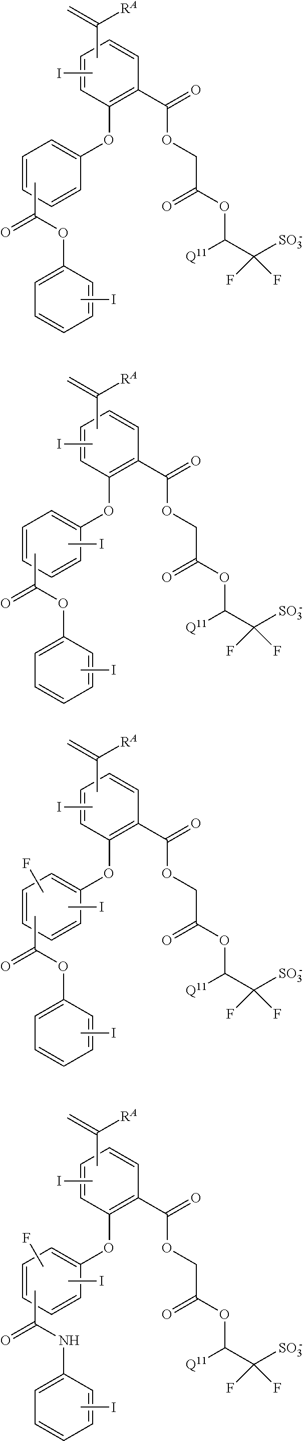

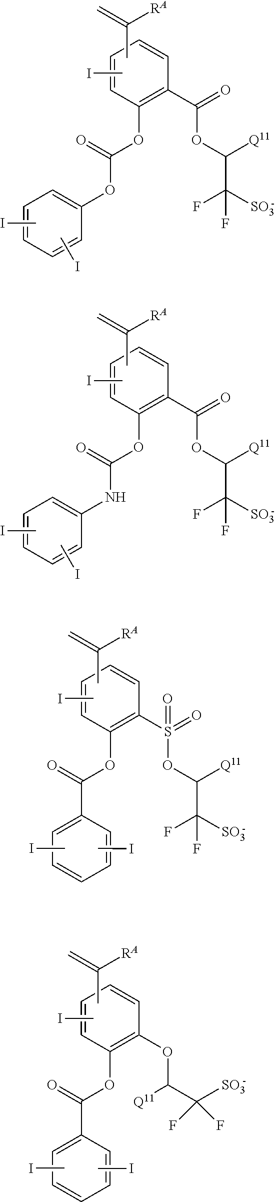

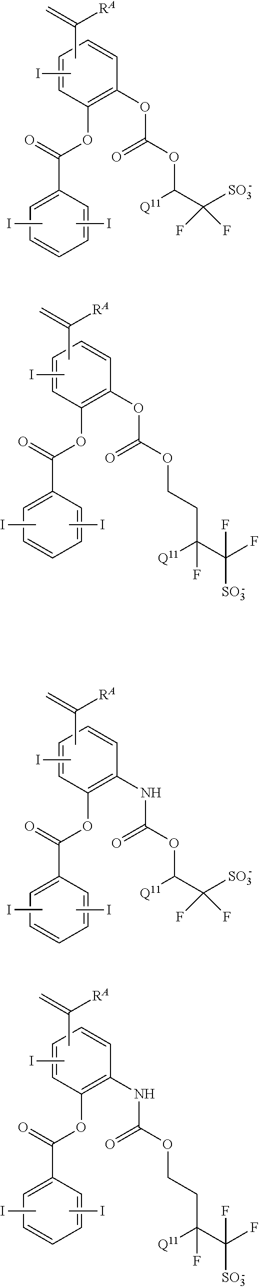

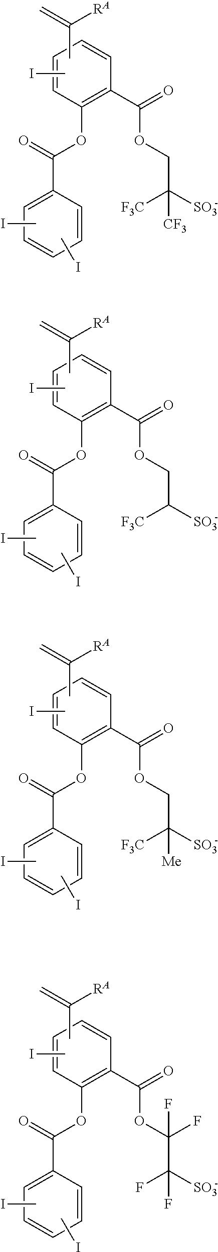

Specific examples of the sulfonium cation of repeating unit d include, but are not limited to, those shown as examples of the sulfonium cation of the sulfonium salt monomer represented by the general formula (a), those shown in paragraphs [0102] to [0125] of JP 2024-3744 A, those shown in paragraphs [0044] to [0049] of WO2024/128017 A1, and those shown in paragraphs [0035] to [0046] of JP 7491173 B.

A sulfonium cation represented by the following general formula (d3) is also preferred as the sulfonium cation of repeating unit d:

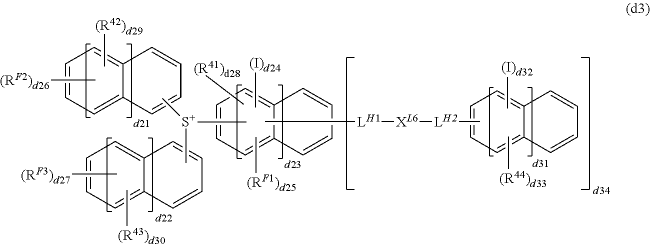

In the general formula (d3), d21 is 0 or 1. While a benzene ring is given if d21 is 0, and a naphthalene ring is given if d21 is 1, the case of d21 is 0 to give a benzene ring is preferred for solubility in solvents. d22 is 0 or 1. While a benzene ring is given if d22 is 0, and a naphthalene ring is given if d22 is 1, the case that d22 is 0 to give a benzene ring is preferred for solubility in solvents. d23 is 0 or 1. While a benzene ring is given if d23 is 0, and a naphthalene ring is given if d23 is 1, the case that d23 is 0 to give a benzene ring is preferred for solubility in solvents.

In the general formula (d3), d24 is 0, 1, 2, 3, or 4. More iodine atoms in the cation structure result in enhanced absorption particularly to EUV, but in poor solubility in solvents, which may disadvantageously cause precipitation in the resist composition, and for this reason d24 is preferably 0, 1, 2, or 3, and more preferably 0, 1, or 2.

In the general formula (d3), d25 is 0, 1, 2, 3, or 4. d25 is preferably 0, 1, 2, or 3, and more preferably 0, 1, or 2 for raw material procurement. d26 is 0, 1, 2, 3, 4, 5, or 6. d26 is preferably 0, 1, 2, or 3, and more preferably 0, 1, or 2 for raw material procurement. d27 is 0, 1, 2, 3, 4, 5, or 6. d27 is preferably 0, 1, 2, or 3, and more preferably 0, 1, or 2 for raw material procurement.

In the general formula (d3), d28 is 0, 1, or 2. d28 is preferably 0 or 1 for raw material procurement. d29 is 0, 1, or 2. d29 is preferably 0 or 1 for raw material procurement. d30 is 0, 1, or 2. d30 is preferably 0 or 1 for raw material procurement.

In the general formula (d3), d31 is 0 or 1. While a benzene ring is given if d31 is 0, and a naphthalene ring is given if d31 is 1, the case that d31 is 0 to give a benzene ring is preferred for solubility in solvents.

In the general formula (d3), d32 is 0, 1, 2, 3, or 4. More iodine atoms in the cation structure result in enhanced absorption particularly to EUV, but in poor solubility in solvents, which may disadvantageously cause precipitation in the resist composition, and for this reason d32 is preferably 0, 1, 2, or 3, and more preferably 0, 1, or 2.

In the general formula (d3), d33 is 0, 1, or 2. d33 is preferably 0 or 1 for raw material procurement. d34 is 0, 1, or 2. d34 is preferably 0 or 1 in terms of synthesis.

It should be noted: that 0≤d26+d29≤4 is satisfied if d21 is 0, and 0≤d26+d29≤6 is satisfied if d21 is 1; that 0≤d27+d30≤4 is satisfied if d22 is 0, and 0 d27+d30≤6 is satisfied if d22 is 1; that 1≤d24+d25+d28+d34≤4 is satisfied if d23 is 0, and 1≤d24+d25+d28+d4≤6 is satisfied if d23 is 1; that 0≤d32+d33≤4 is satisfied if d31 is 0, and 0≤d32+d33≤6 is satisfied if d31 is 1; and that d24+d32≥1 is satisfied.

In the general formula (d3), R^{F1} to RF³ are each independently a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms. Among them, R^{F1} to R^{F3} are preferably each a trifluoromethyl group, a trifluoromethoxy group, or a trifluorothiomethoxy group. If d25 is 2 or more, R^{F1} groups are identical to or different from each other; if d26 is 2 or more, R^{F2} groups are identical to or different from each other; and if d27 is 2 or more, R^{F3} groups are identical to or different from each other.

In the general formula (d3), R⁴¹ to R⁴⁴ are each a halogen atom being nether an iodine atom nor a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. The hydrocarbyl group and the hydrocarbyl moieties of the hydrocarbyloxy group and hydrocarbylthio group may be each saturated or unsaturated, and may be each linear, branched, or cyclic. Specific examples thereof include those shown as examples of the hydrocarbyl group represented by R¹ in the description of the general formula (a). Some or all of the hydrogen atoms of the hydrocarbyl group and the hydrocarbyl moieties of the hydrocarbyloxy group and hydrocarbylthio group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

If d28 is 2, the two R⁴¹ groups are identical to or different from each other, and the two R⁴¹ groups are optionally bound together to form a ring together with the carbon atoms to which the two groups are bound; if d29 is 2, the two R⁴² groups are identical to or different from each other, and the two R⁴² groups are optionally bound together to form a ring together with the carbon atoms to which the two groups are bound; if d30 is 2, the two R⁴³ groups are identical to or different from each other, and the two R⁴³ groups are optionally bound together to form a ring together with the carbon atoms to which the two groups are bound; and if d33 is 2, the two R⁴⁴ groups are identical to or different from each other, and the two R⁴⁴ groups are optionally bound together to form a ring together with the carbon atoms to which the two groups are bound. Specific examples of the ring formed in this case include a cyclopropane ring, a cyclobutane ring, a cyclopentane ring, a cyclohexane ring, a norbornane ring, and an adamantane ring. Some or all of the hydrogen atoms in the ring may be each replaced with a group containing a heteroatom such as an oxygen atom, a sulfur atom, a nitrogen atom, and a halogen atom, some of the —CH₂— moieties in the ring may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

Aromatic rings directly bound to S⁺ in the sulfonium cation represented by the general formula (d3) are optionally bound together to form a ring together with S⁺. Specific examples of the structure of the ring in this case include those represented by the following formulae:

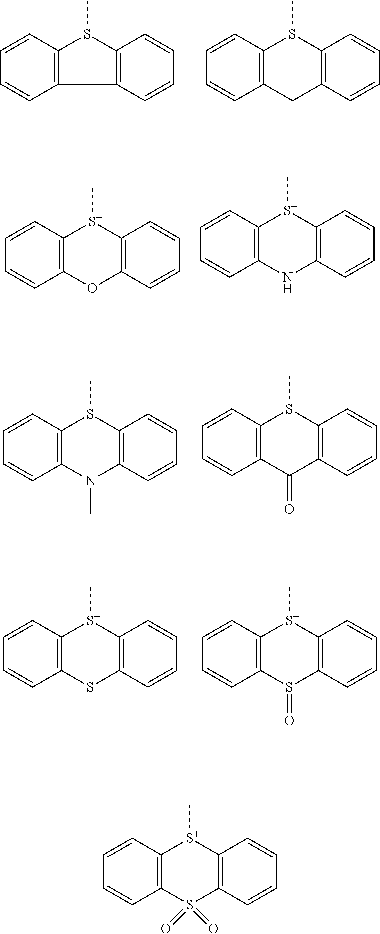

wherein each dashed line indicates bonding.

In the general formula (d3), L^{H1} and L^{H2} are each independently a single bond, an ether bond, an ester bond, an amide bond, a sulfonate bond, a sulfonamide bond, a carbonate bond, or a carbamate bond. Among them, L^{H1} is preferably a single bond, an ether bond, an ester bond, or a sulfonate bond, and more preferably an ester bond or a sulfonate bond. L^{H2} is preferably a single bond, an ether bond, or an ester bond, and more preferably a single bond.

In the general formula (d3), X^{L6} is a single bond or a hydrocarbylene group having 1 to 40 carbon atoms and optionally containing a heteroatom. The hydrocarbylene group may be linear, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom. Specific examples of the hydrocarbylene group represented by X^{L6}, having 1 to 40 carbon atoms and optionally containing a heteroatom, include X^{L}-0 to X^{L}-58, which have been shown as specific examples of the hydrocarbylene group represented by X^{L1} and X^{L2}, having 1 to 40 carbon atoms and optionally containing a heteroatom, in the description of the general formula (Z). Among them, X^{L}-0 to X^{L}-22, X^{L}-29 to X^{L}-34, and X^{L}-47 to X^{L}-58 are preferred.

The sulfonium cation represented by the general formula (d3) is preferably one represented by the following general formula (d3-1):

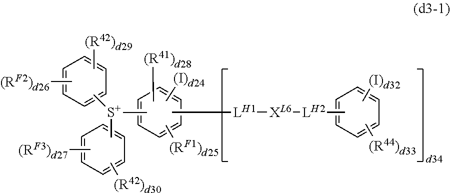

wherein d24 to d30, d32 to d34, R^{F1} to R^{F3}, R⁴¹ to R⁴⁴, L^{H1}, L^{H2}, and X^{L6} are as specified above.

The sulfonium cation represented by the general formula (d3-1) is preferably one represented by the following general formula (d3-2):

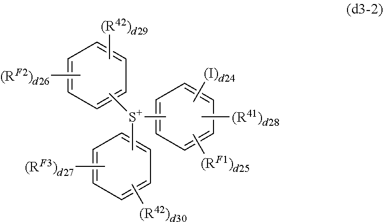

wherein d24 to d30, R^{F1} to R^{F3}, and R⁴¹ to R⁴³ are as specified above.

Specific examples of the sulfonium cation represented by the general formula (d3) include, but are not limited to, those shown below. In the following formulae, Me is a methyl group:

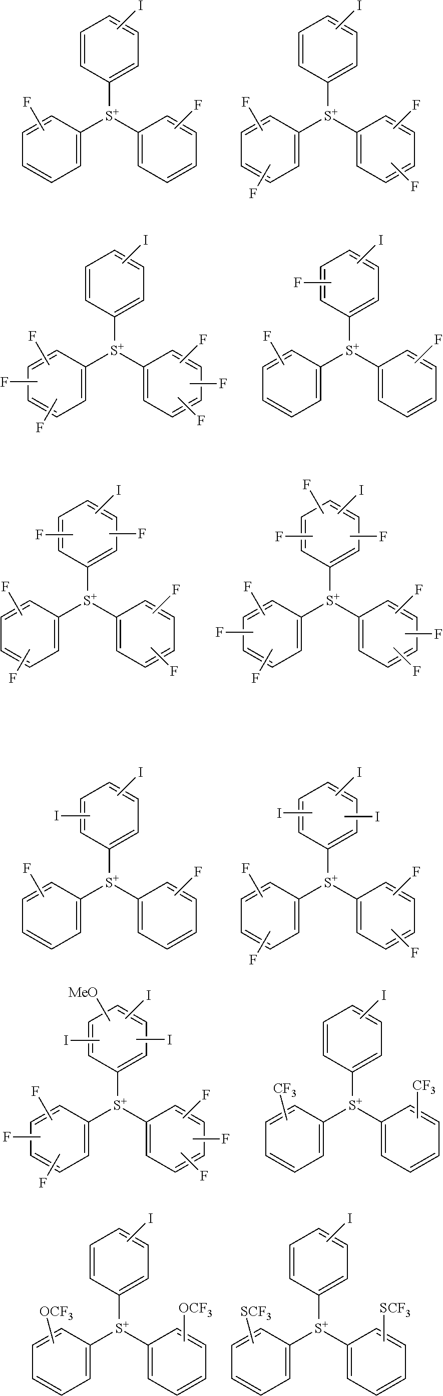

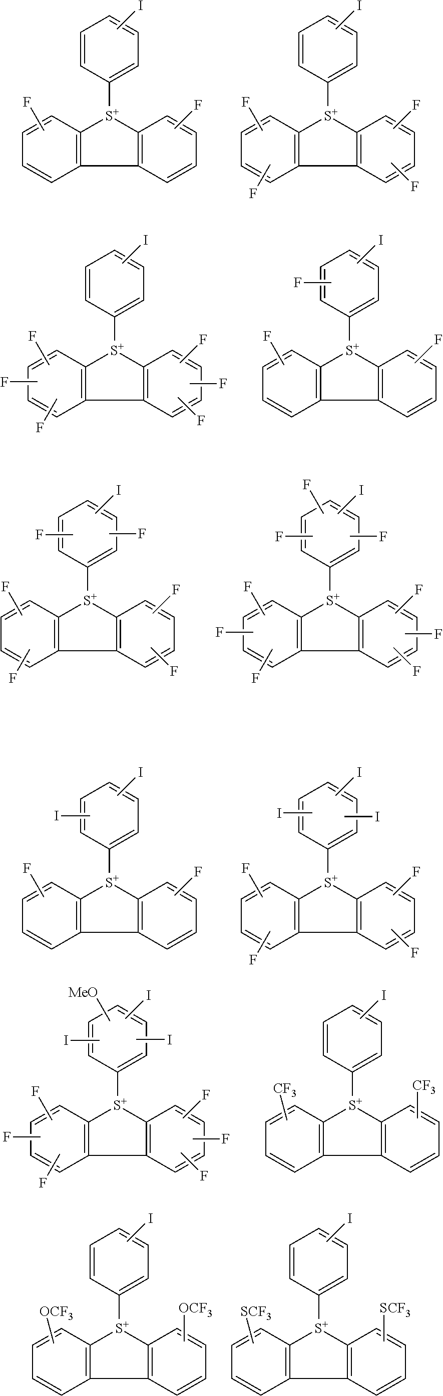

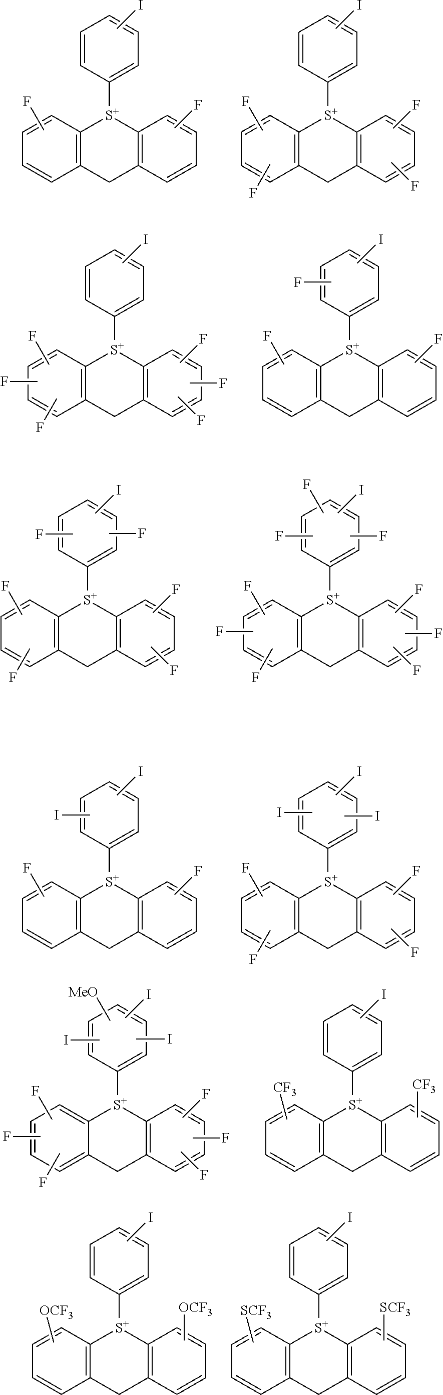

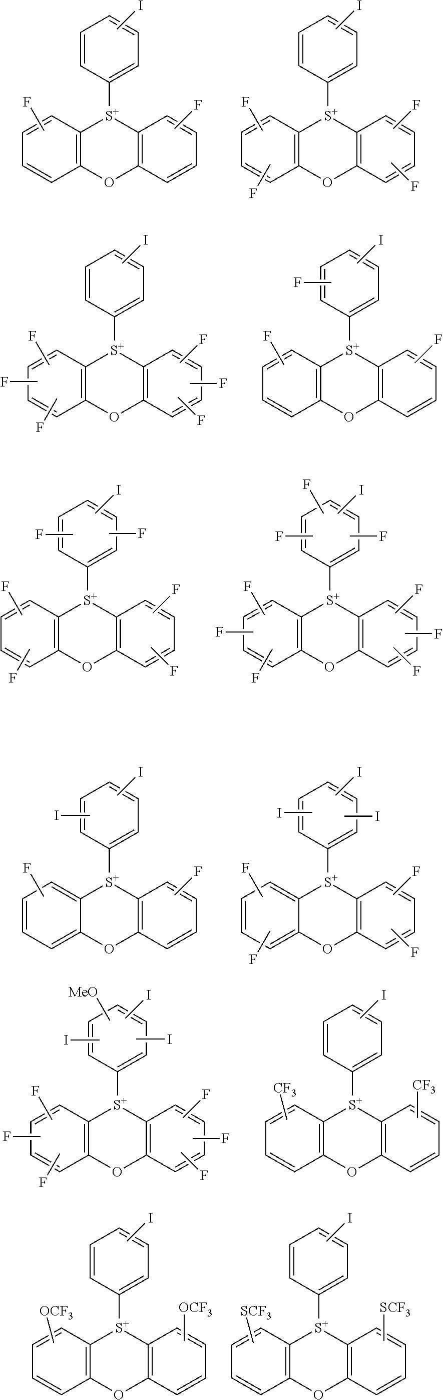

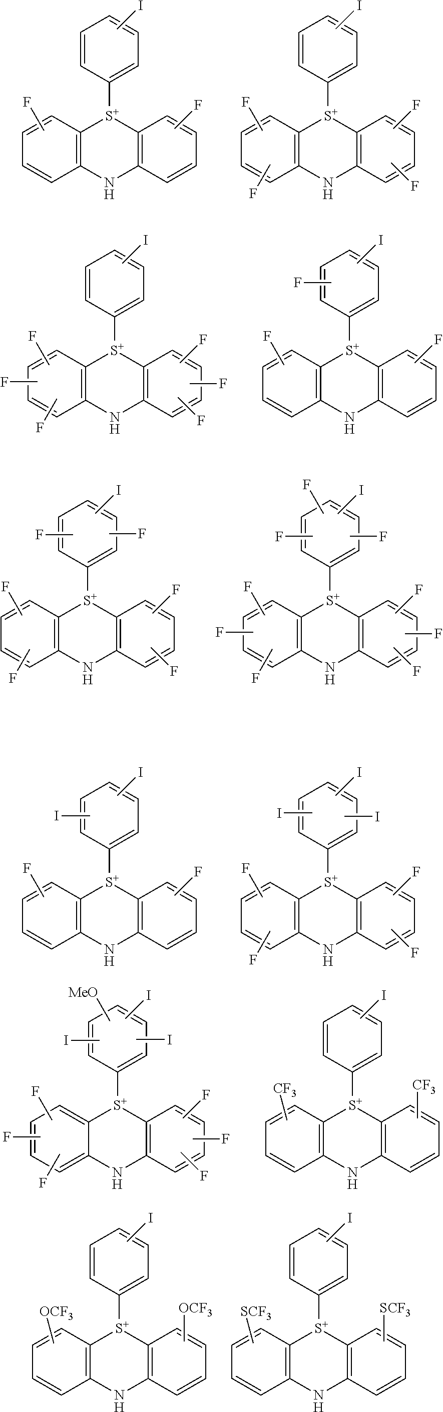

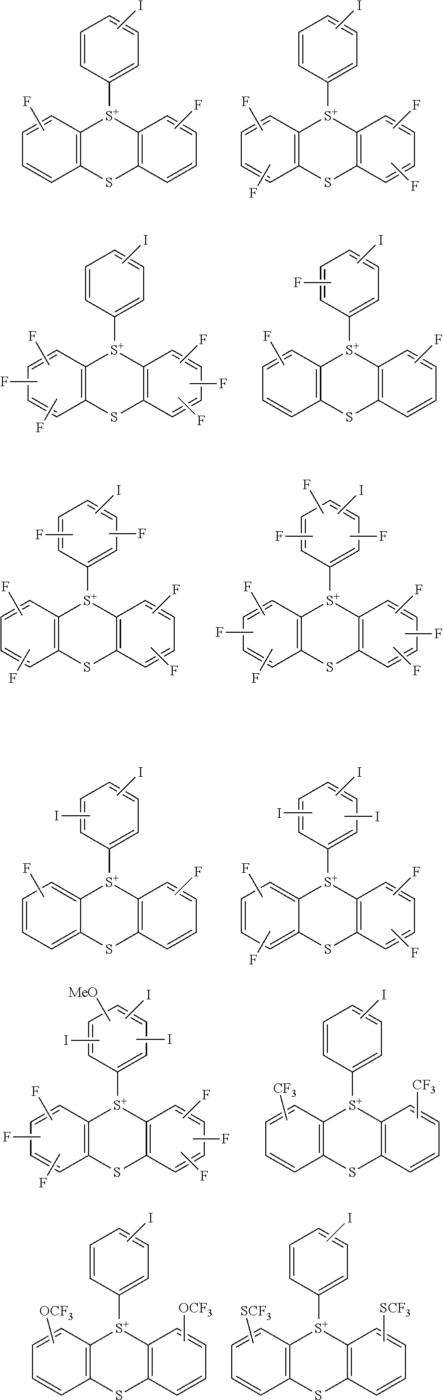

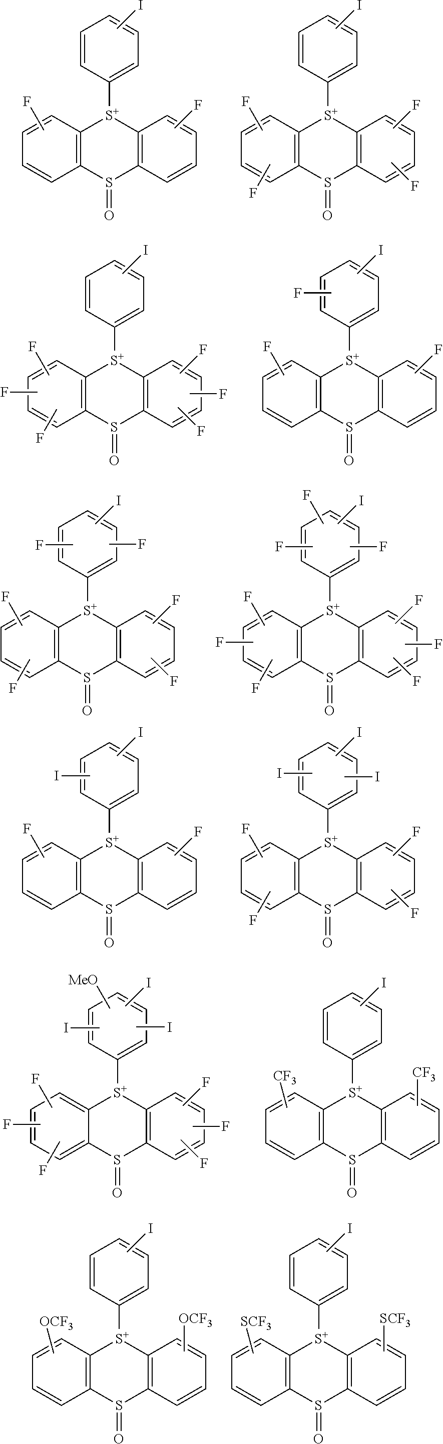

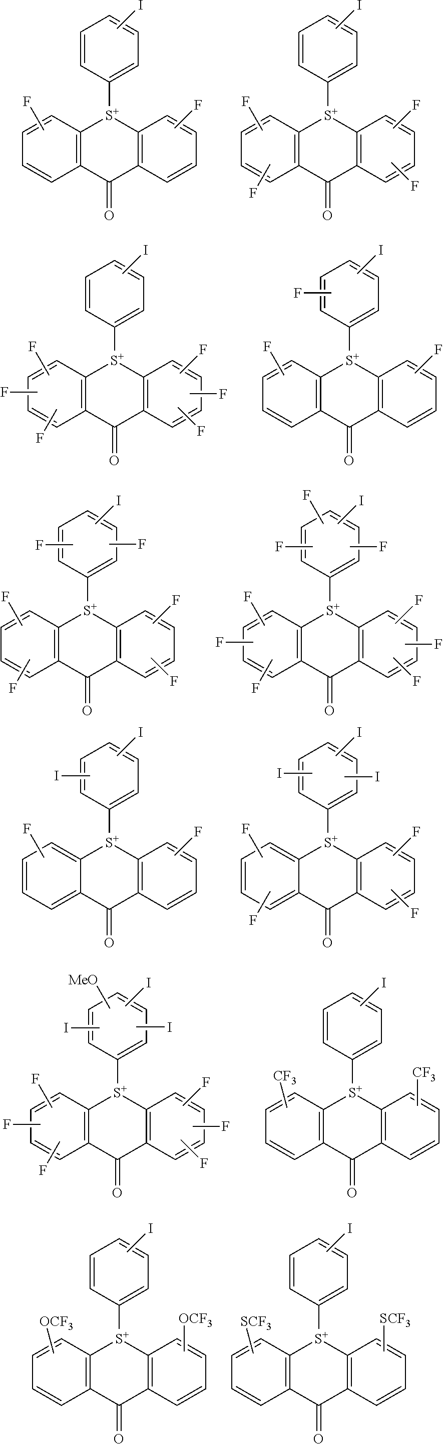

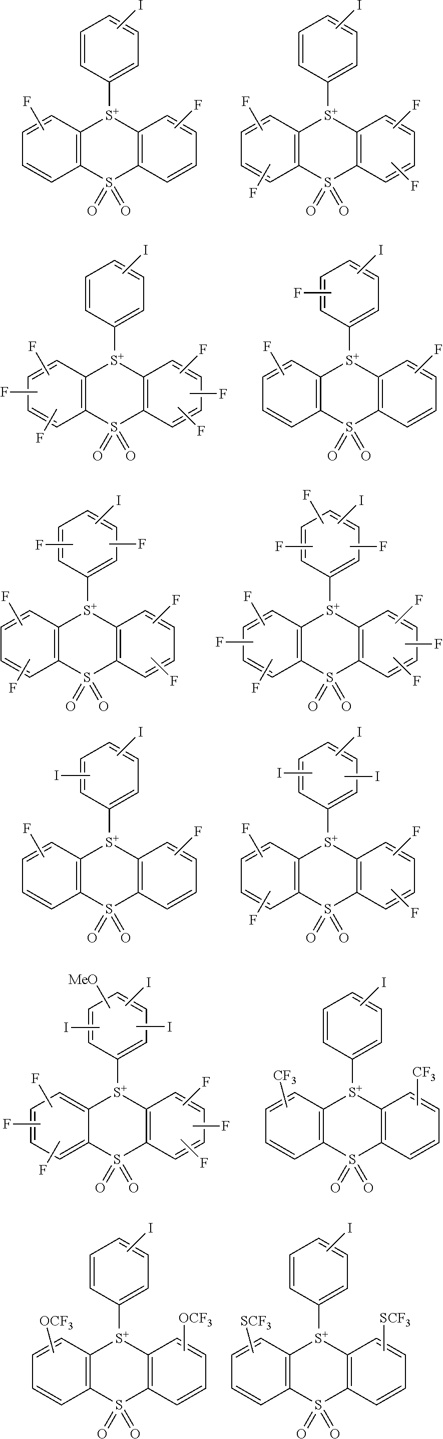

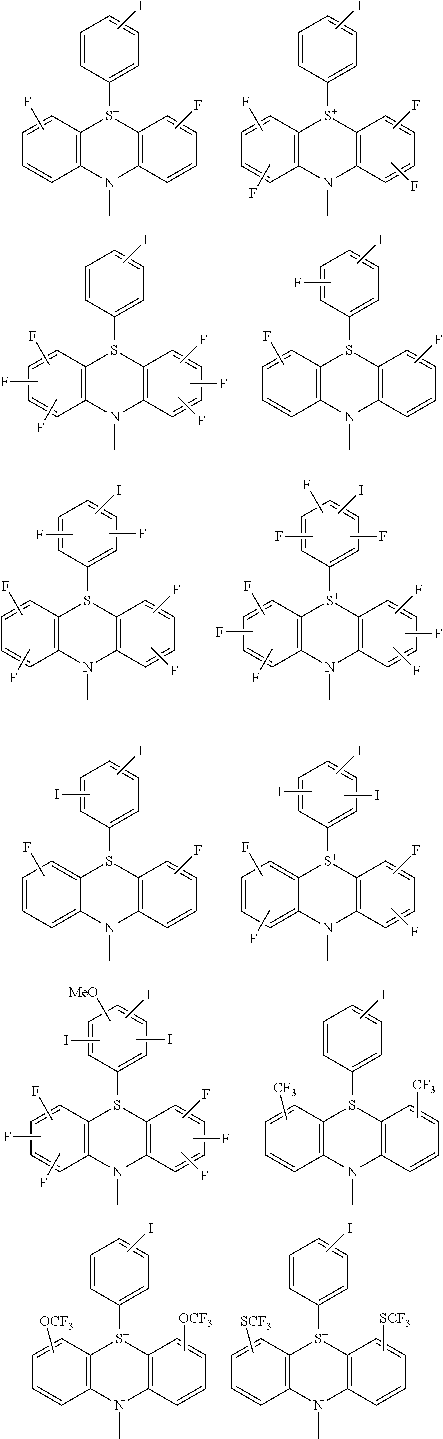

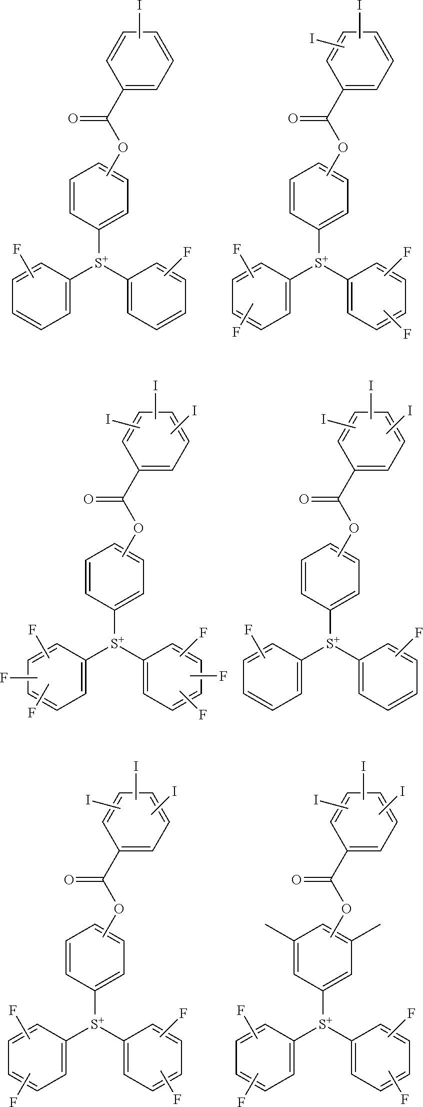

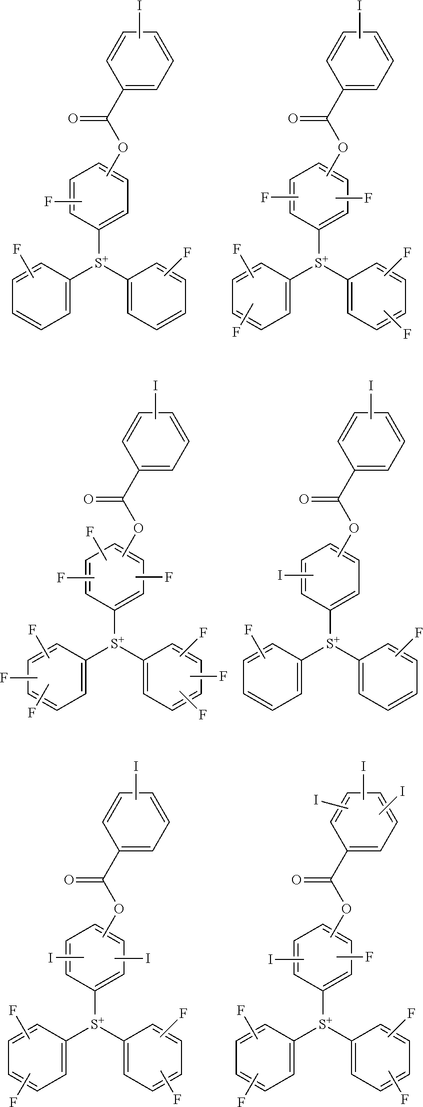

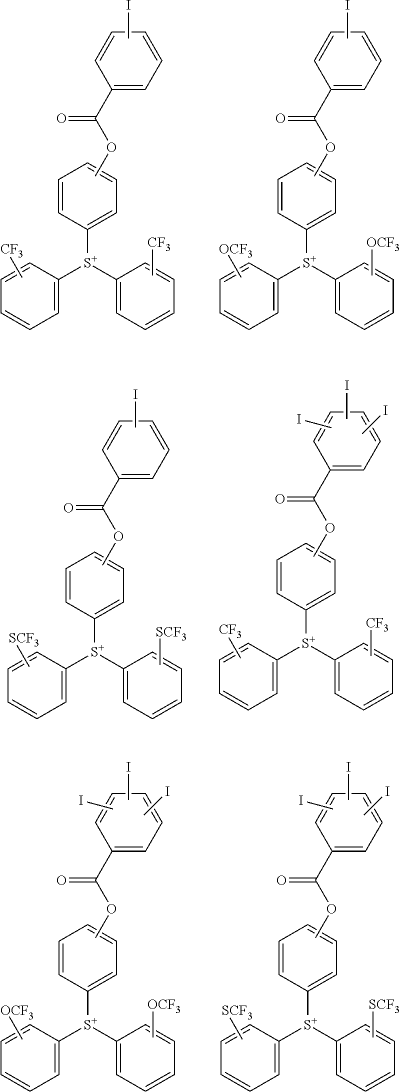

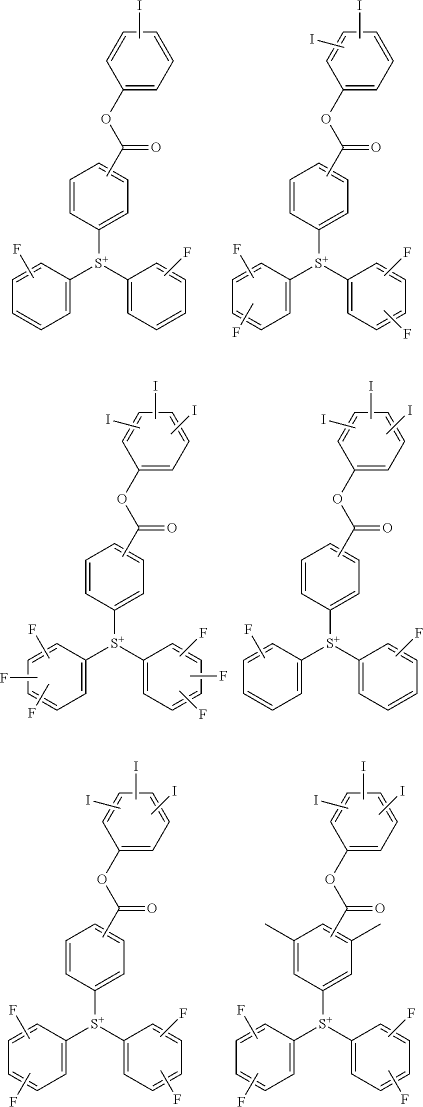

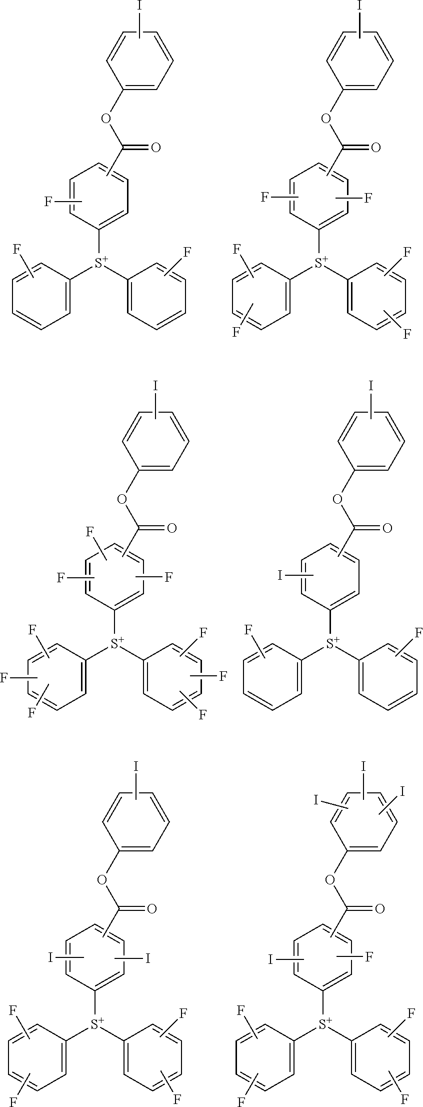

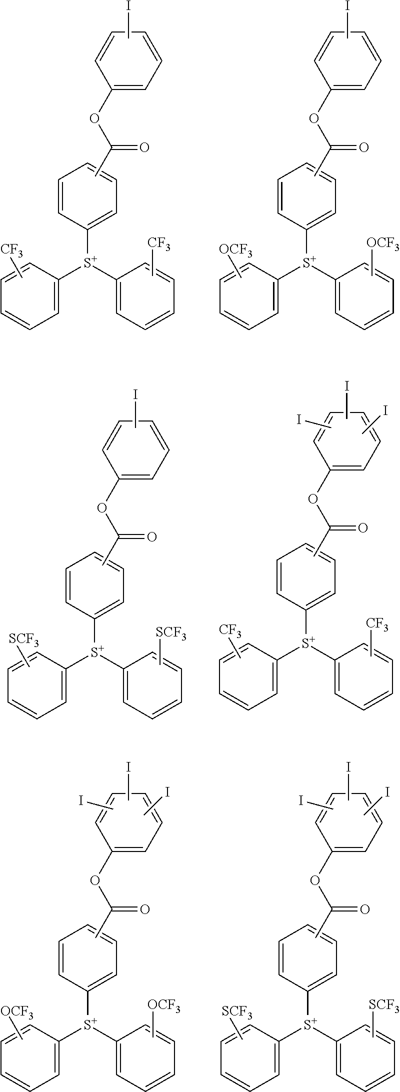

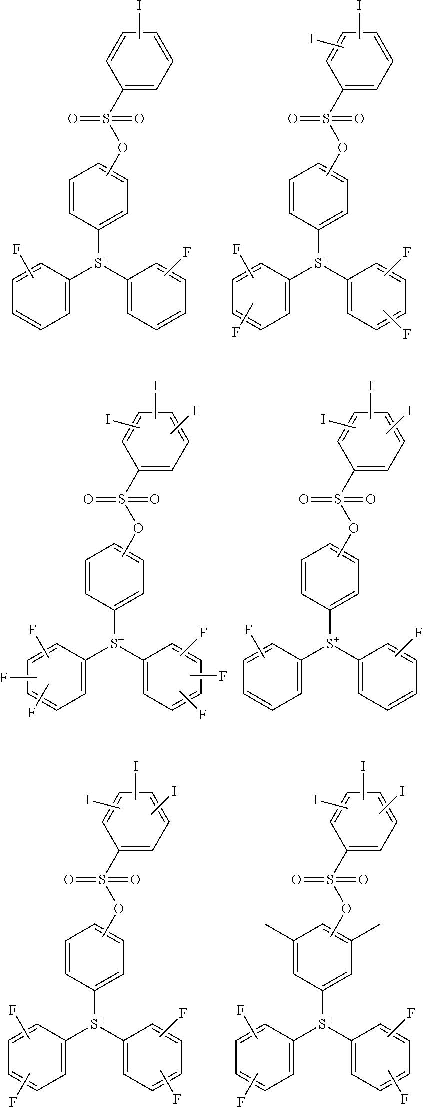

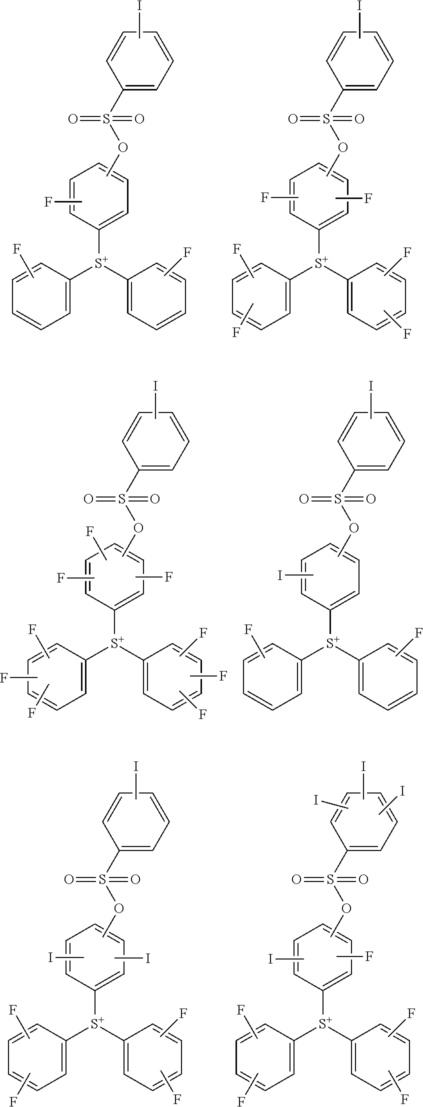

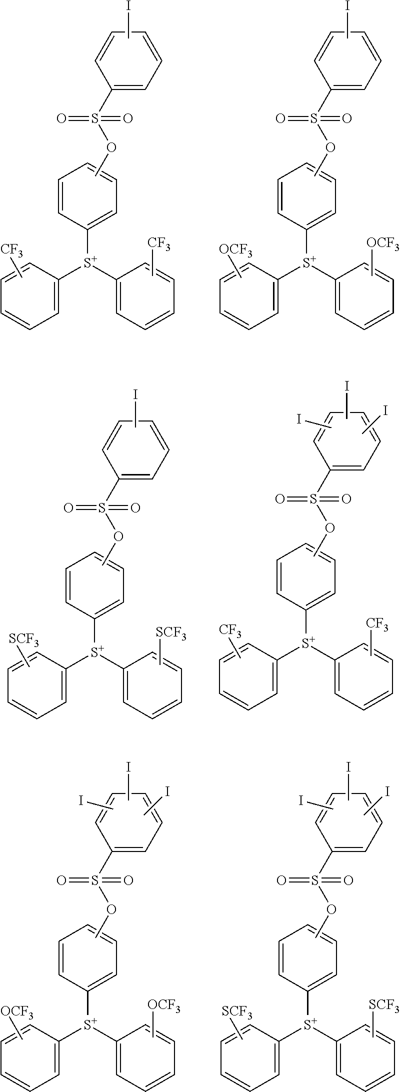

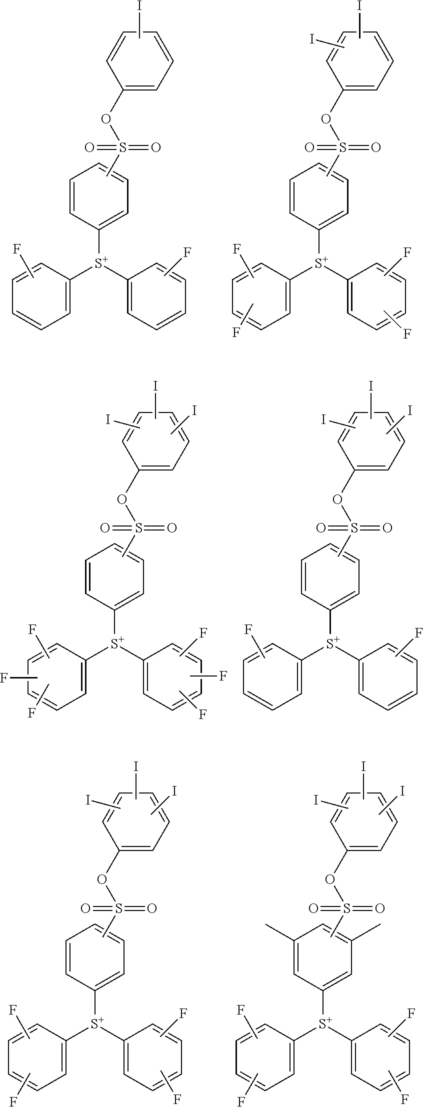

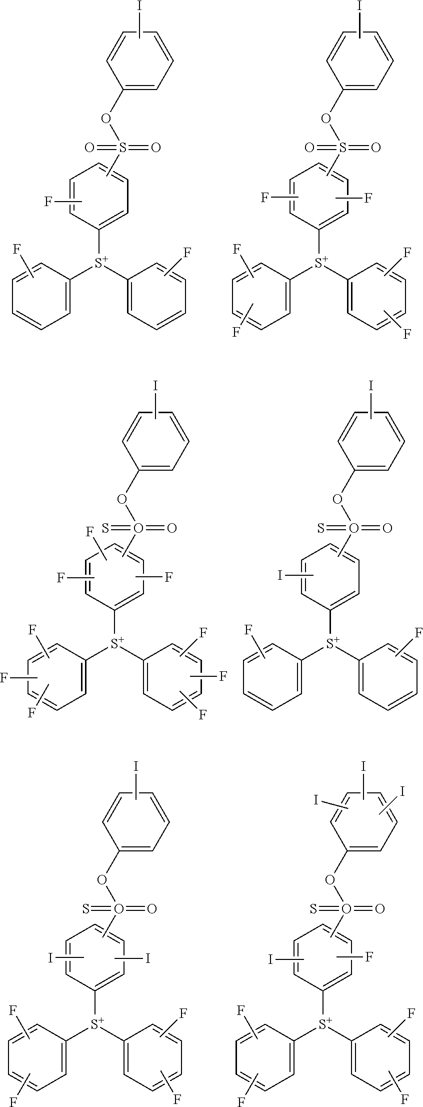

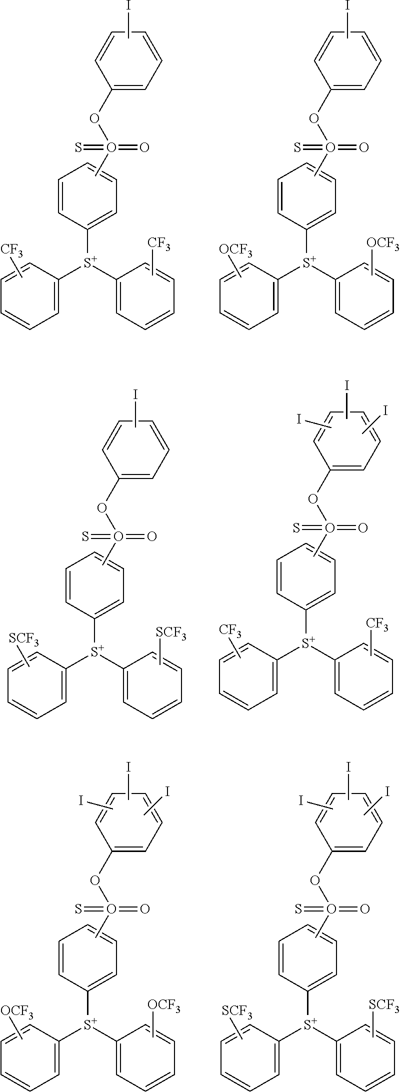

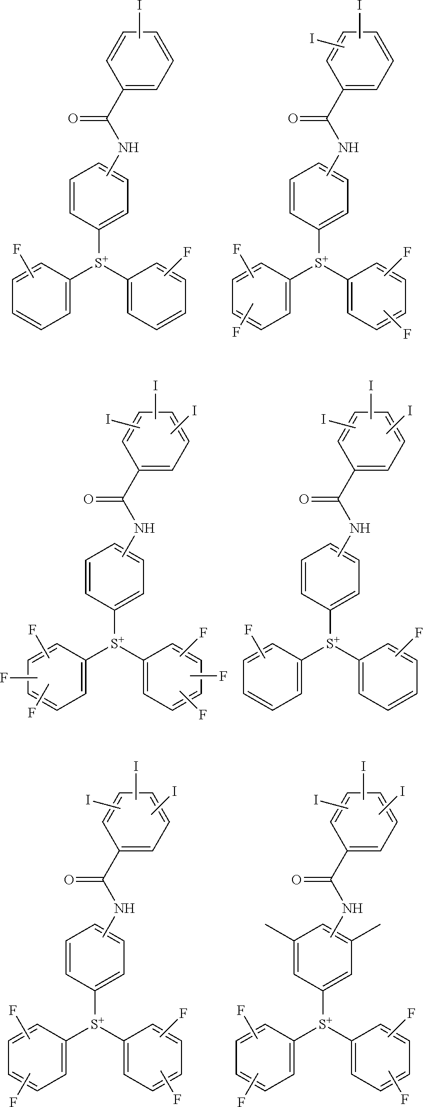

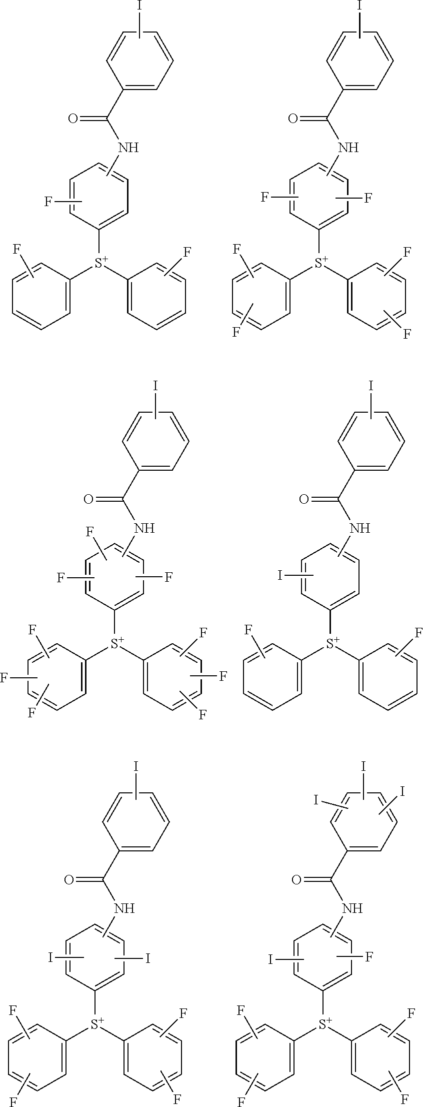

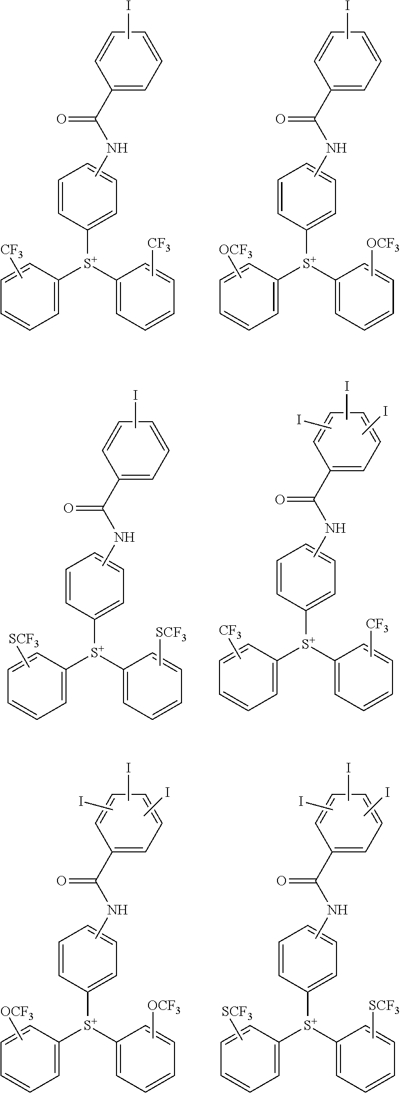

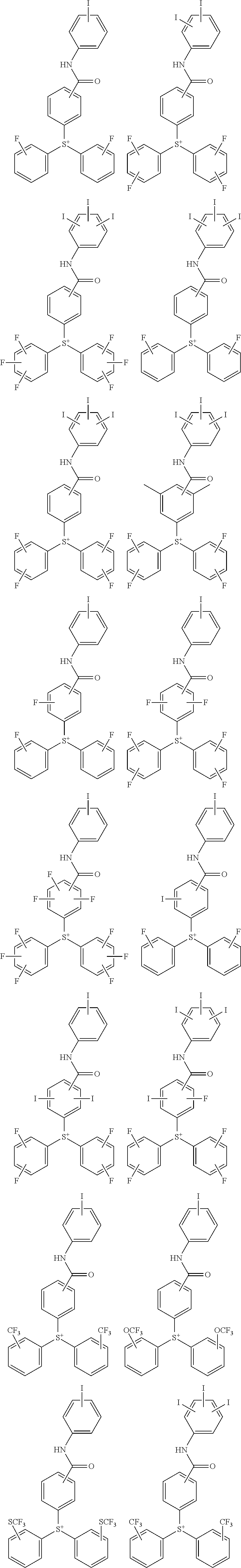

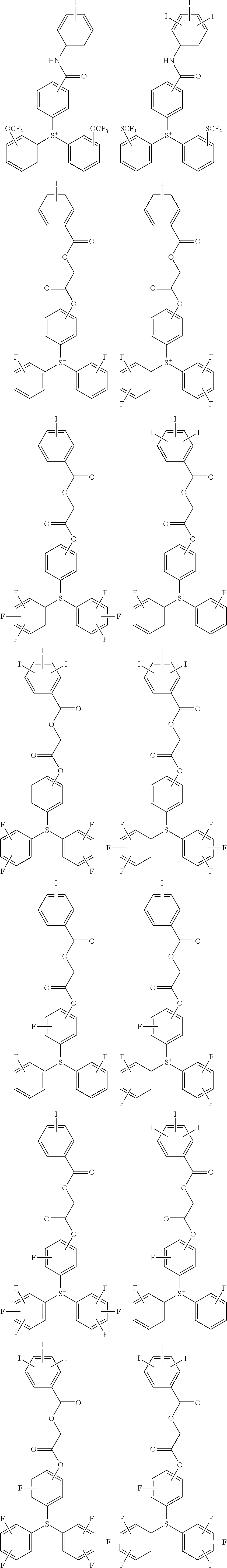

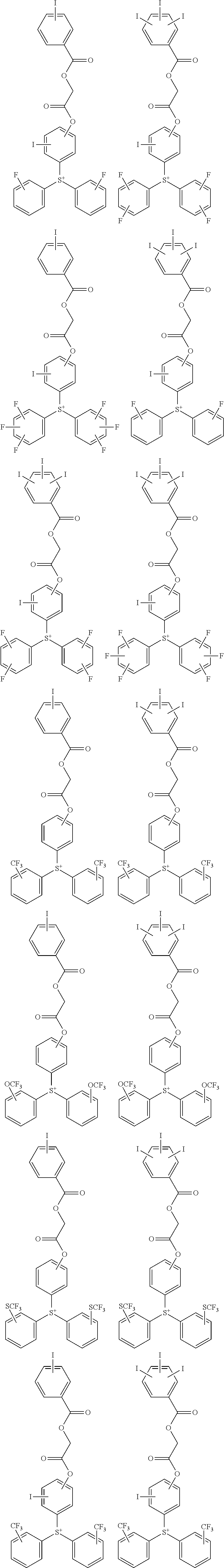

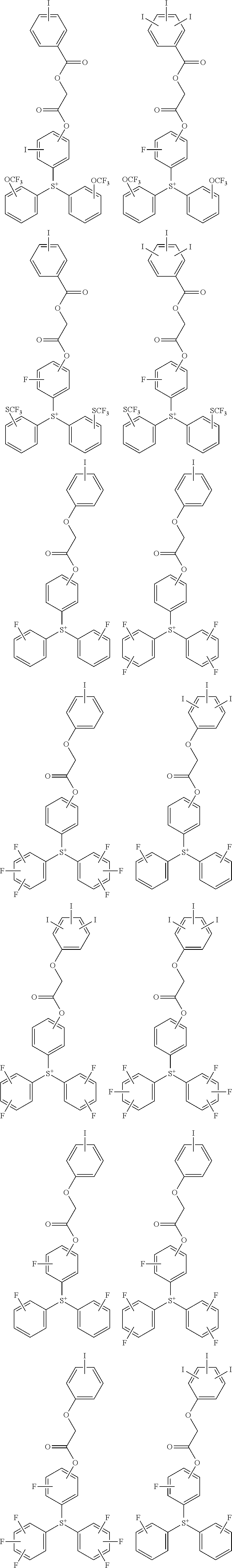

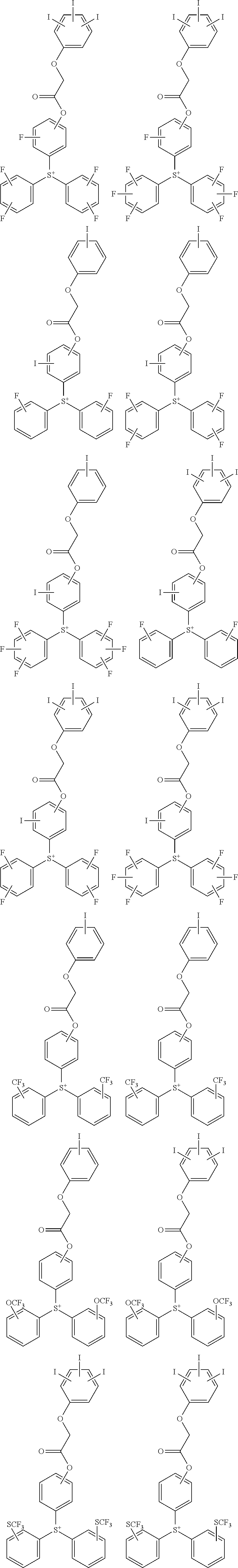

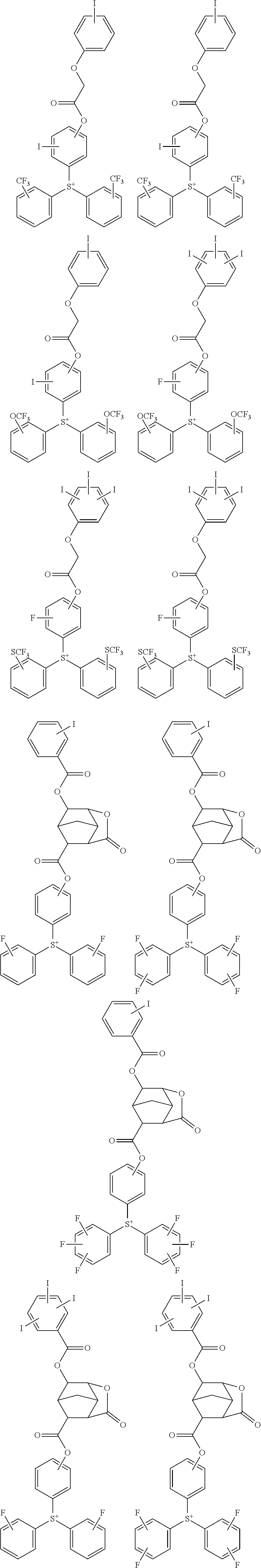

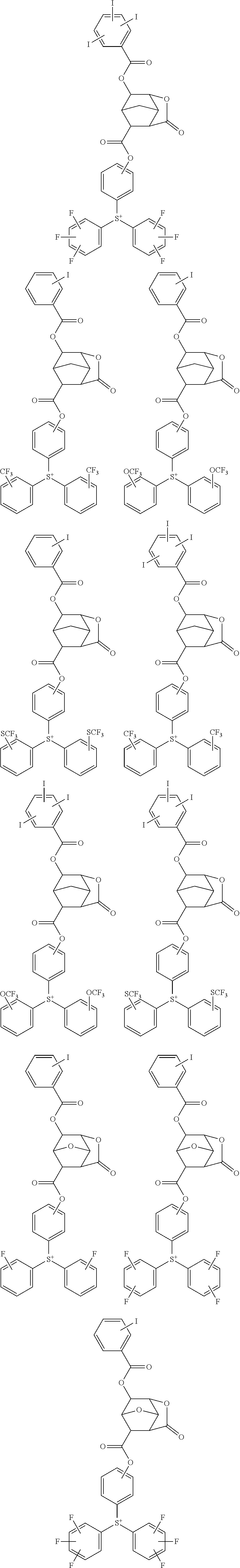

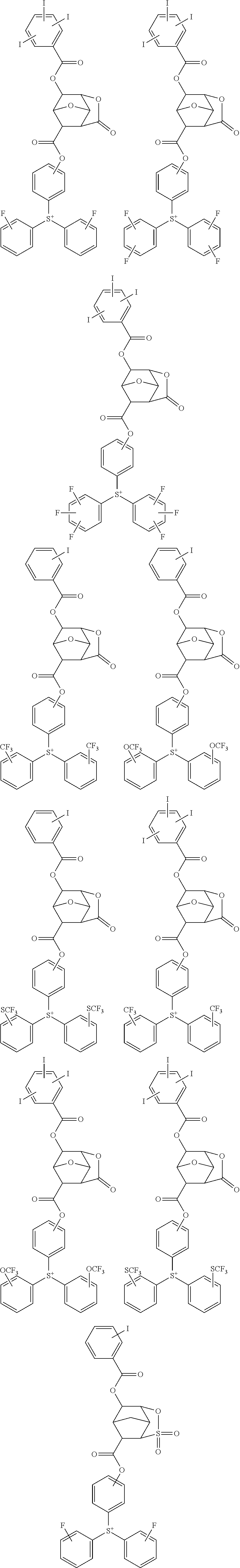

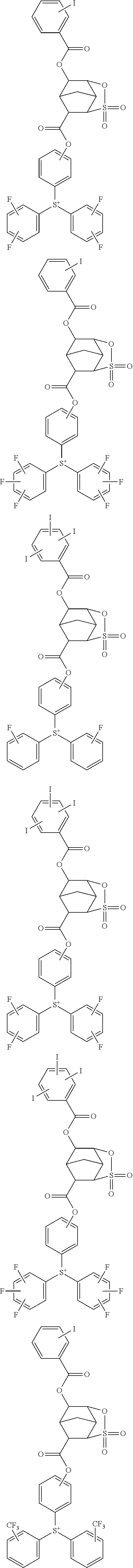

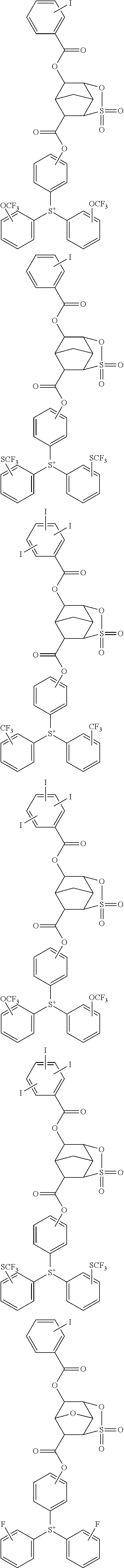

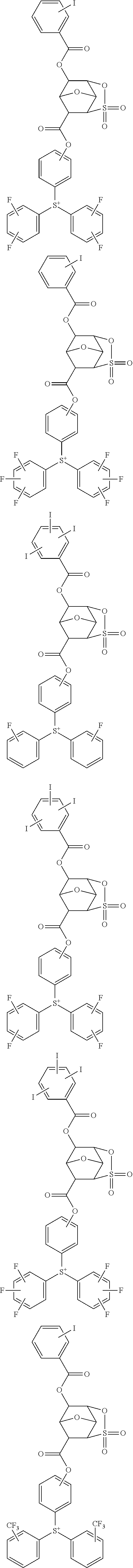

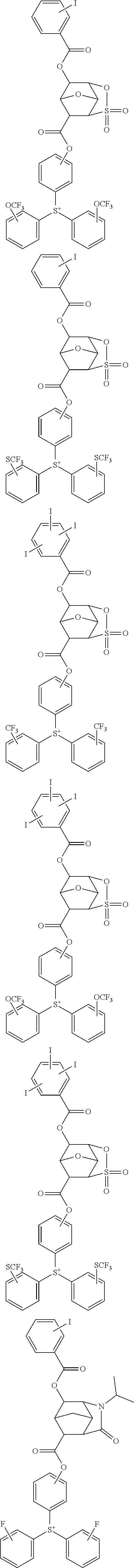

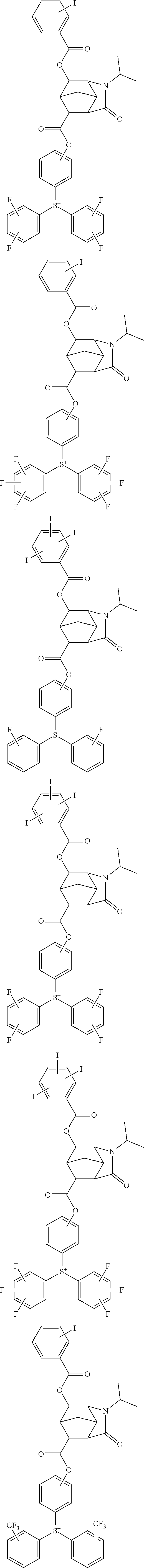

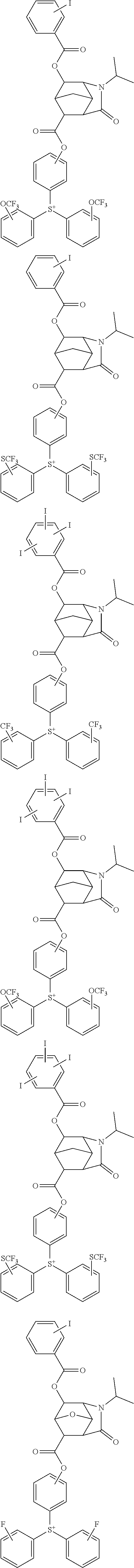

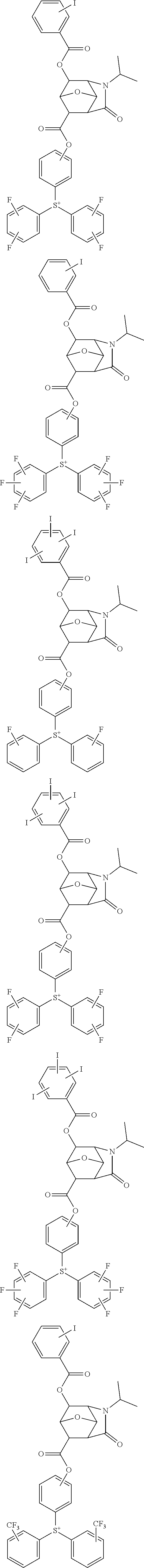

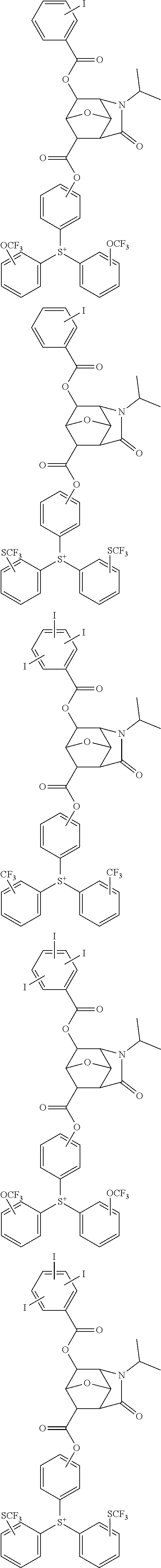

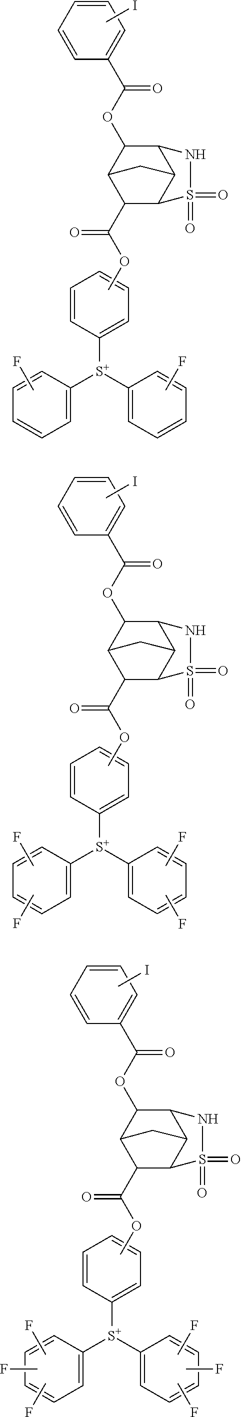

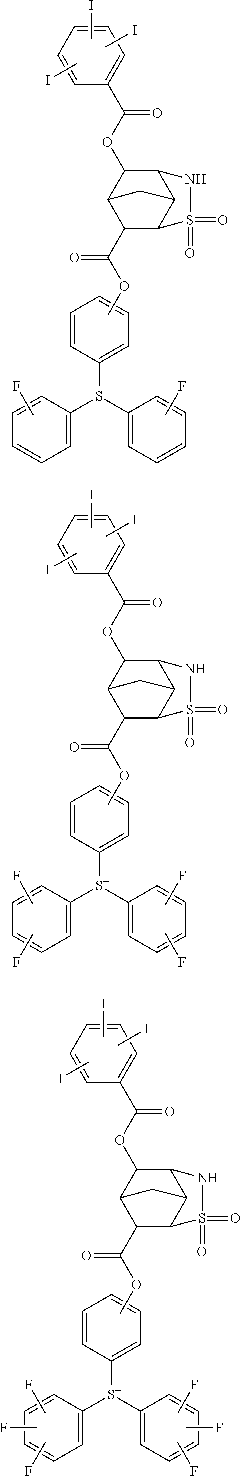

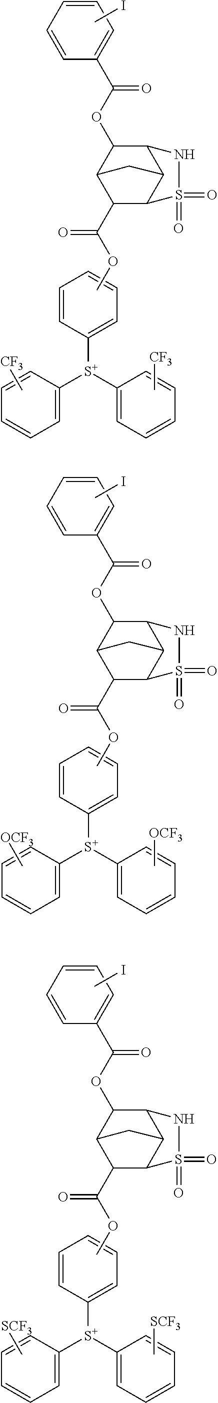

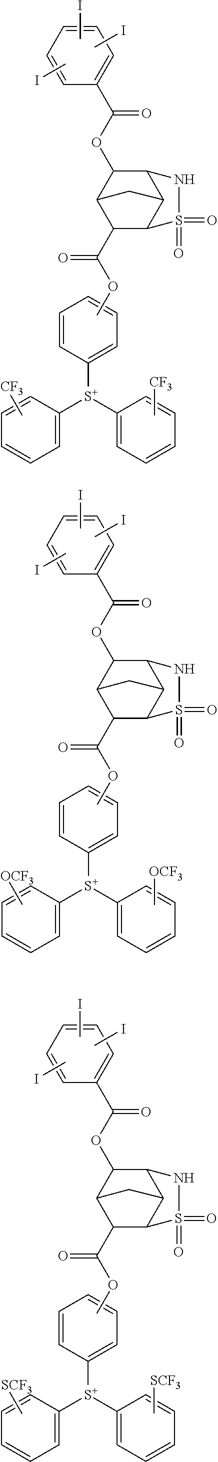

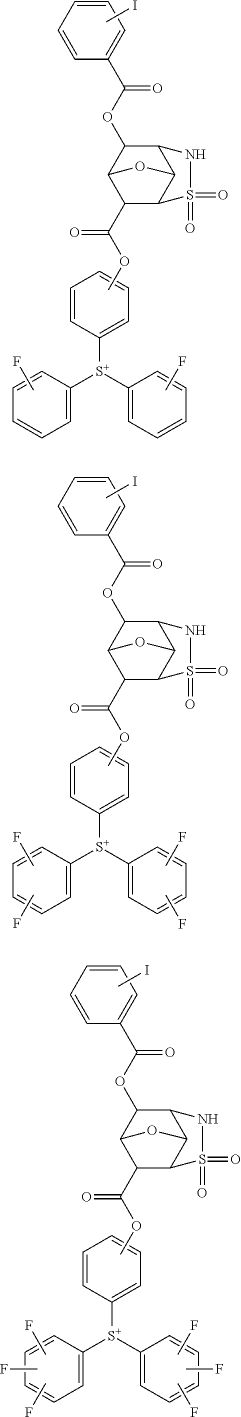

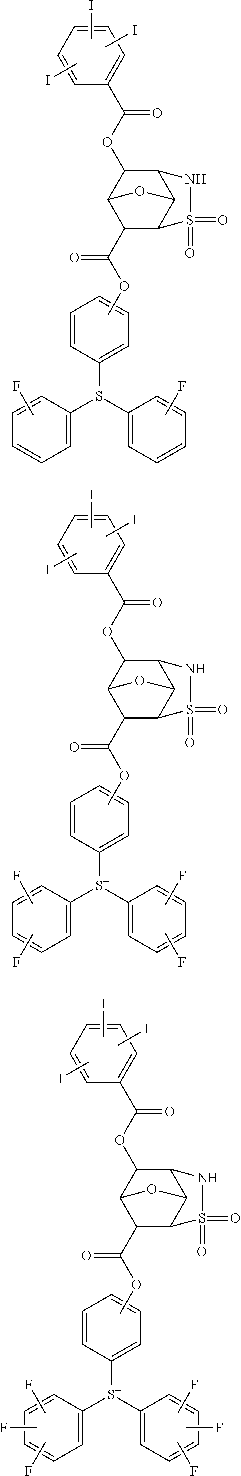

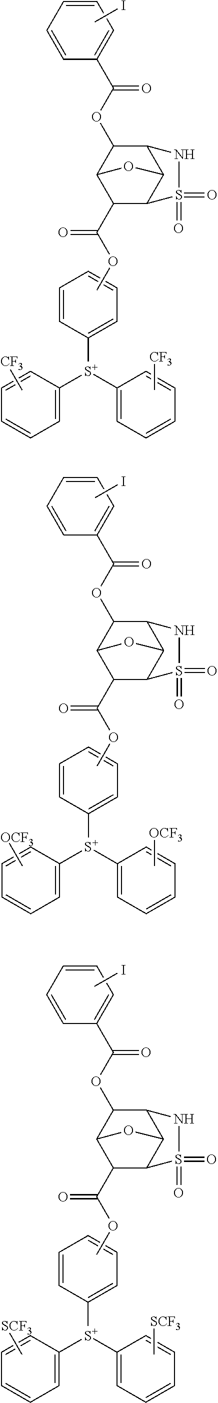

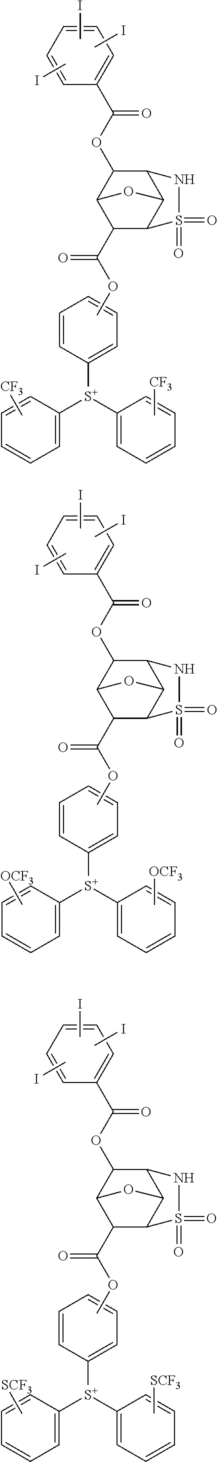

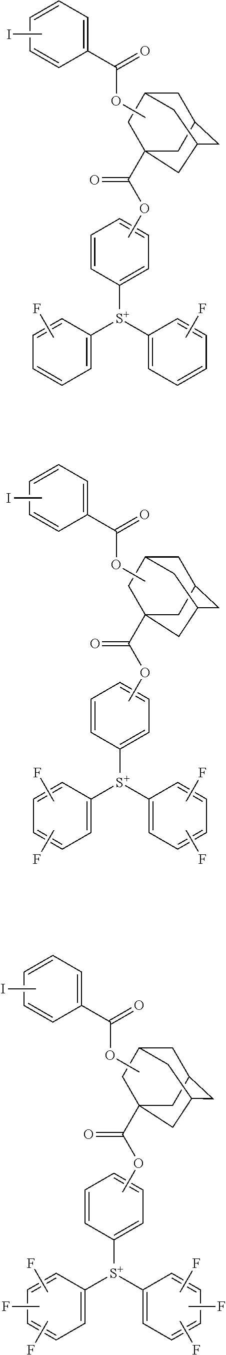

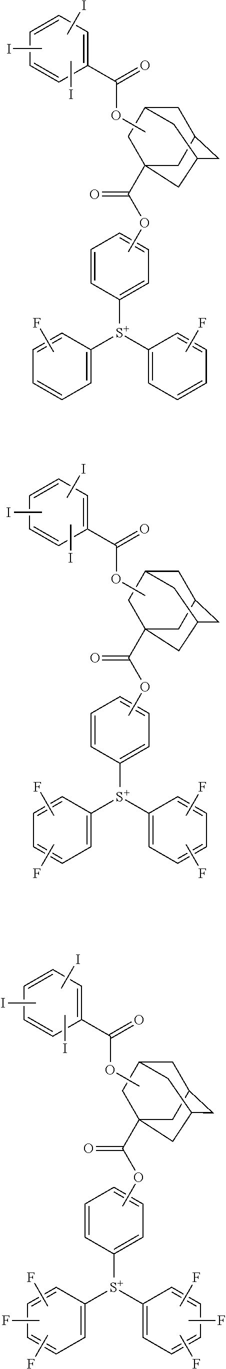

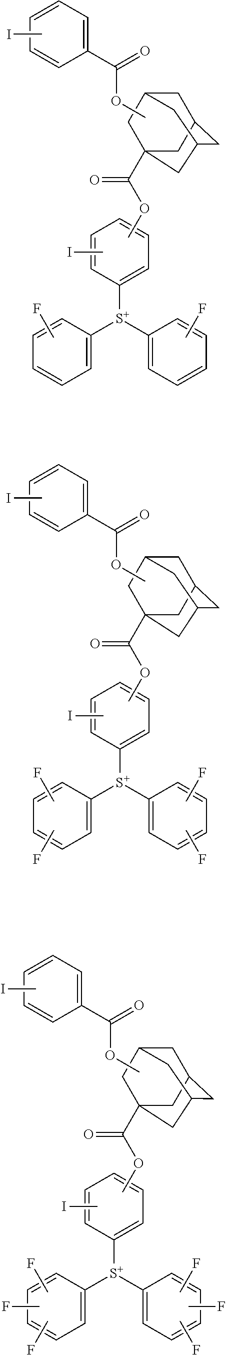

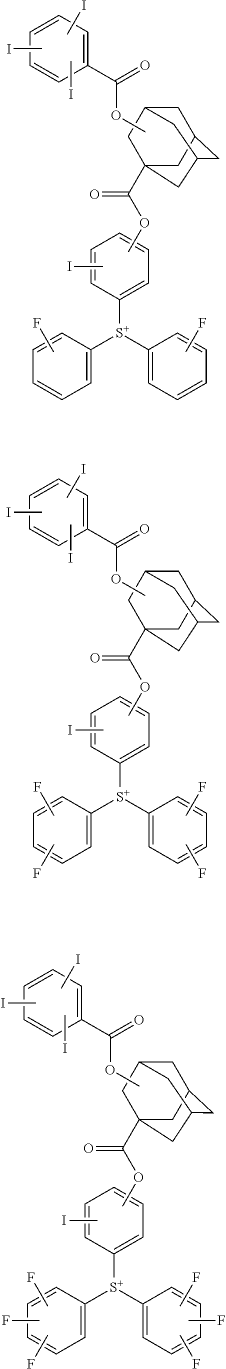

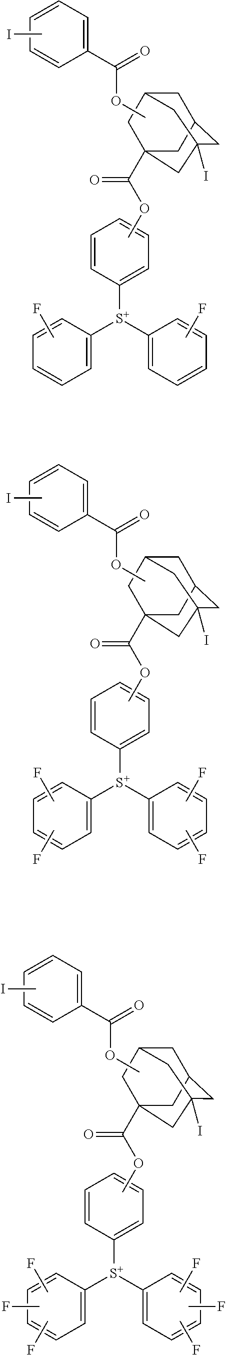

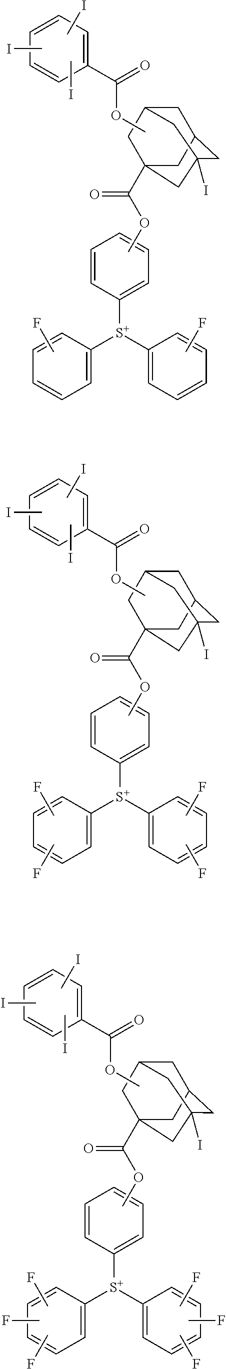

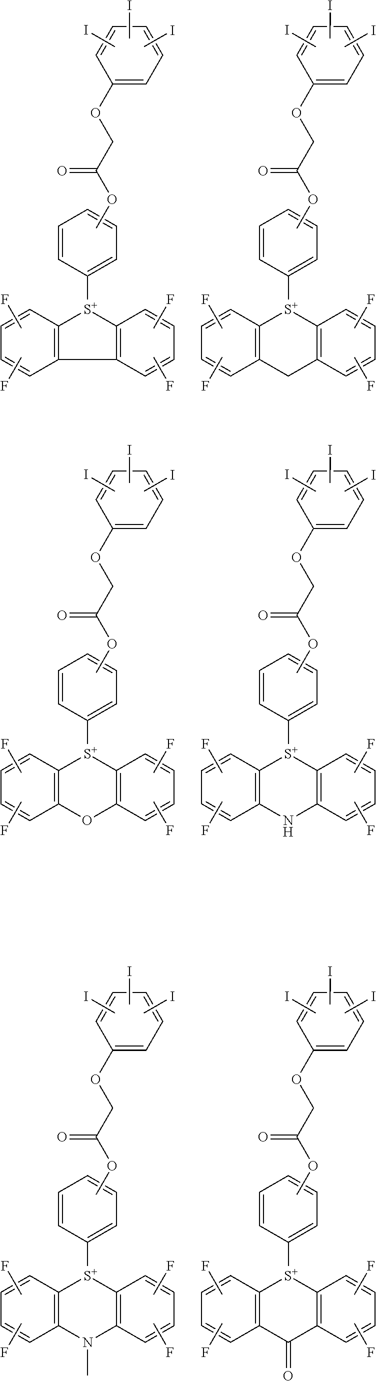

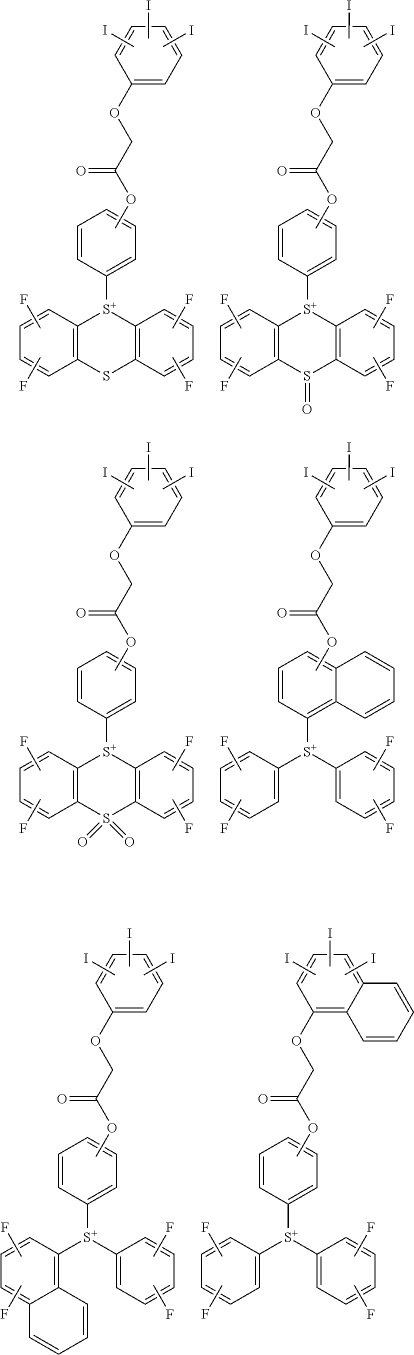

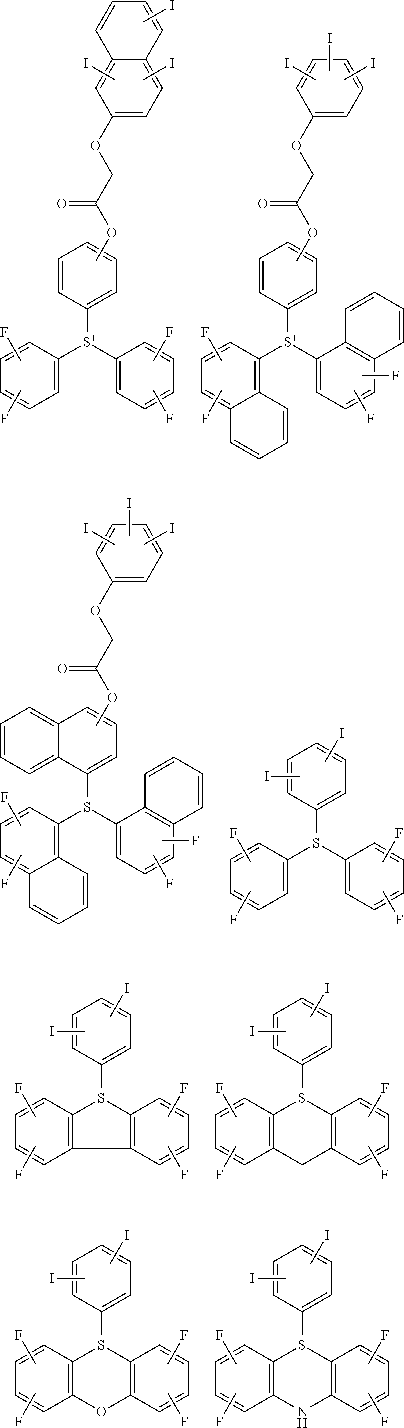

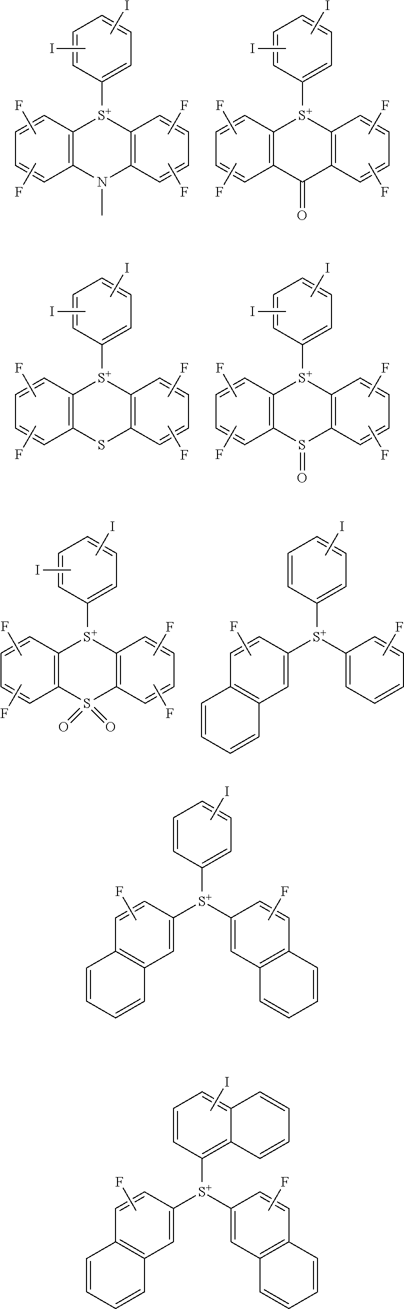

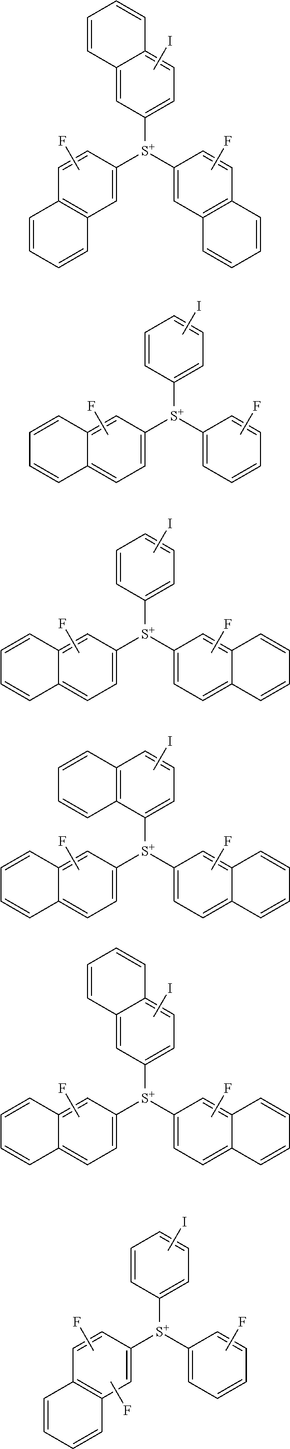

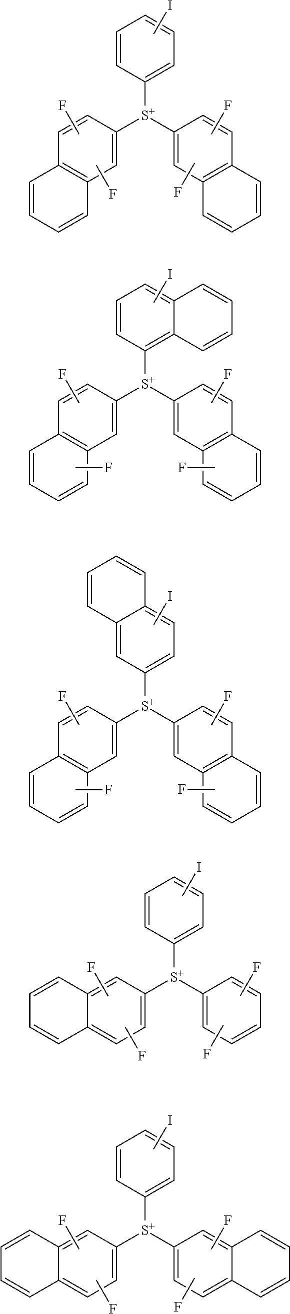

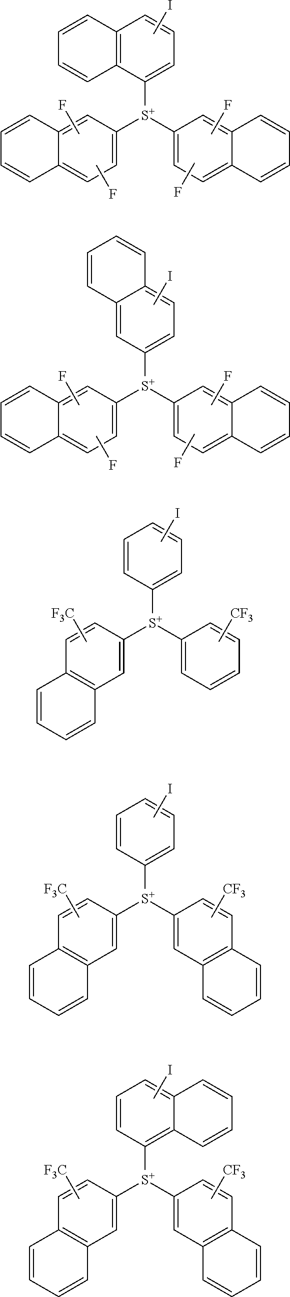

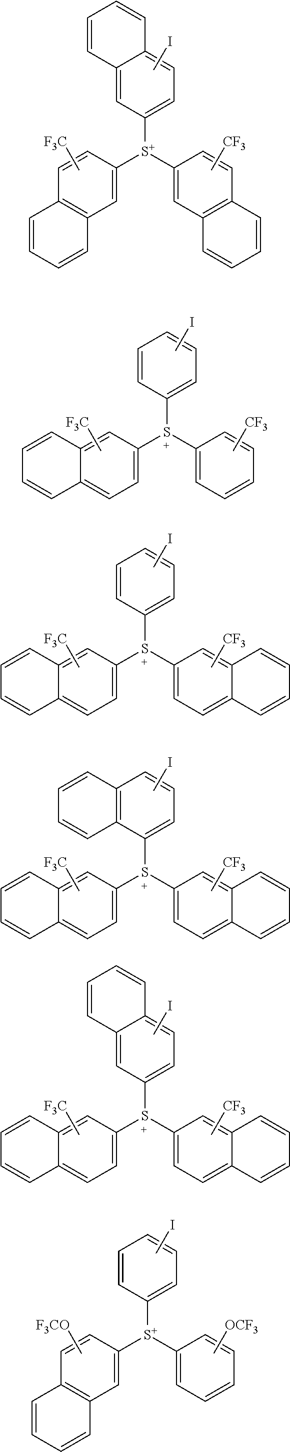

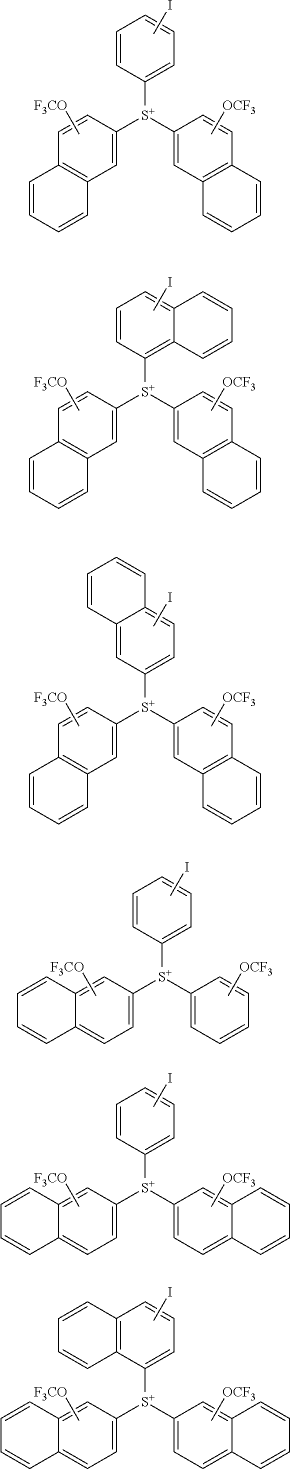

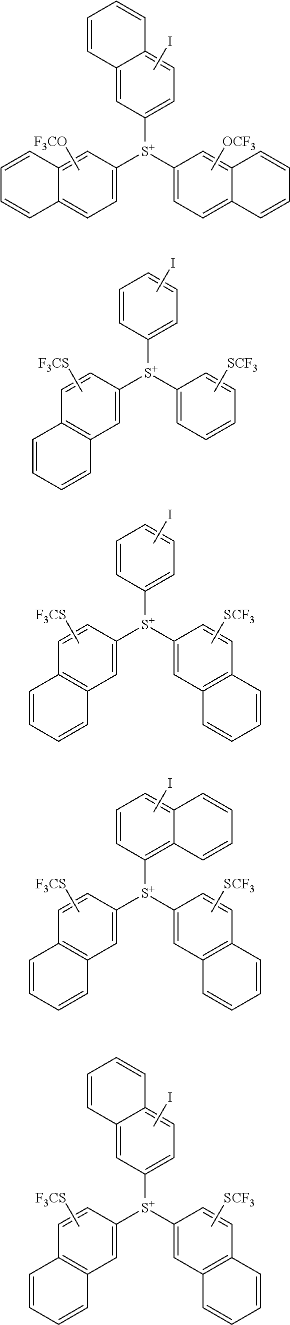

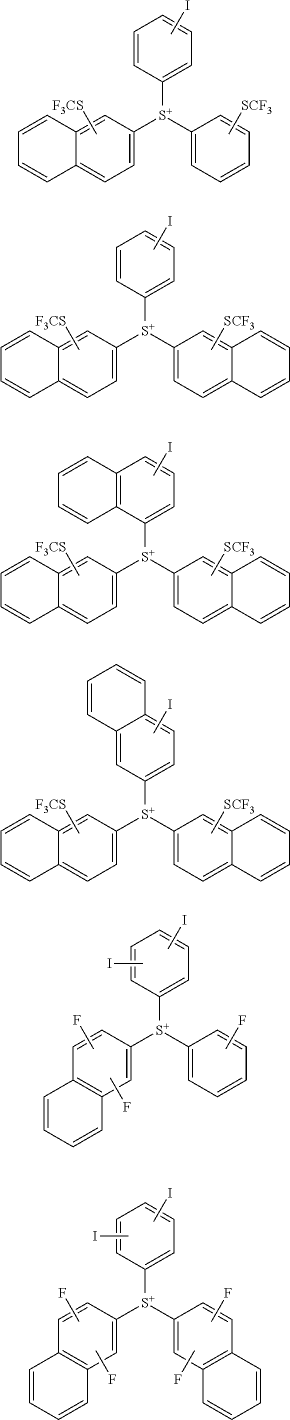

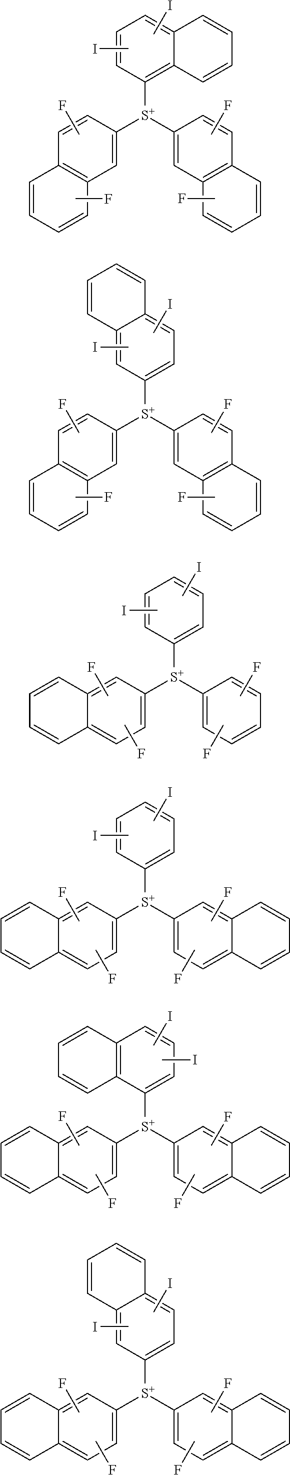

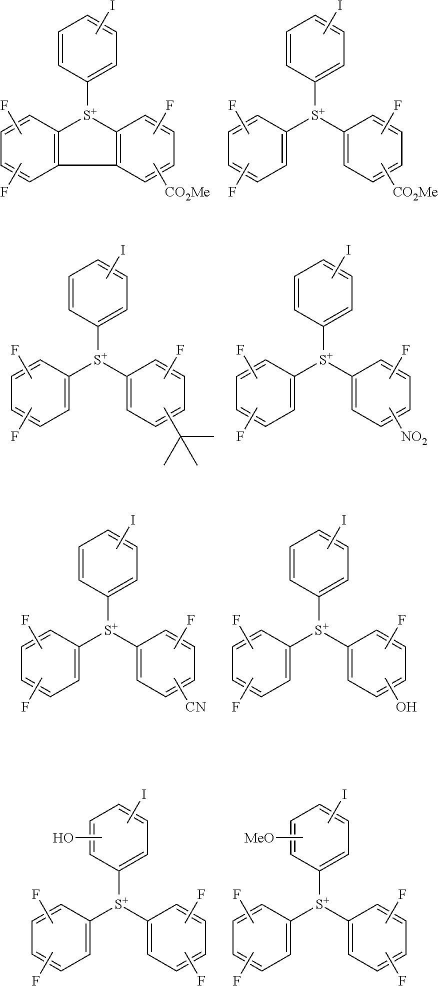

The polymer preferably further contains a repeating unit represented by the following general formula (e) (hereinafter, also referred to as repeating unit e):

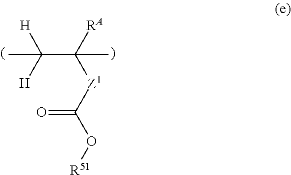

wherein R is a hydrogen atom, a fluorine atom, a methyl group, or a trifluoromethyl group;
Z¹ is a single bond, a phenylene group, a naphthylene group, *—C(═O)—O—Z¹¹—, or *—C(═O)—NH—Z¹¹—, wherein the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, * indicates bonding to a carbon atom of a main chain, Z¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring; and
R⁵¹ is a hydrogen atom or a group having 1 to 20 carbon atoms and containing at least one structure selected from a hydroxy group being not a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—).

In the general formula (e), R^{A} is a hydrogen atom, a fluorine atom, a methyl group, or a trifluoromethyl group. Z¹ is a single bond, a phenylene group, a naphthylene group, *—C(═O)—O—Z¹¹—, or *—C(═O)—NH—Z¹¹—, wherein the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom. * indicates bonding to a carbon atom of a main chain. Z¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring. R⁵¹ is a hydrogen atom or a group having 1 to 20 carbon atoms and containing at least one structure selected from a hydroxy group being not a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—).

Specific examples of repeating unit e include, but are not limited to, those shown below. In the following formulae, R^{A} is as specified above:

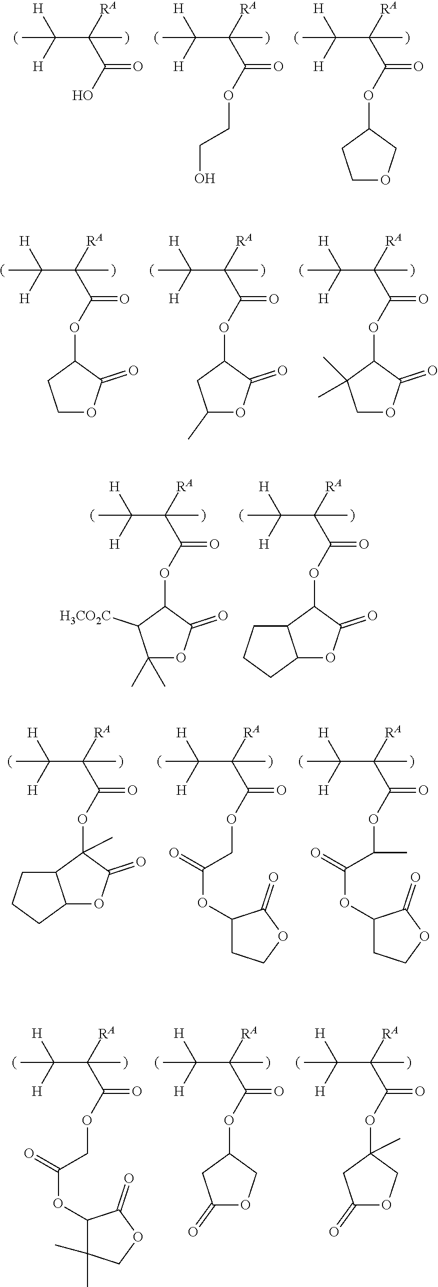

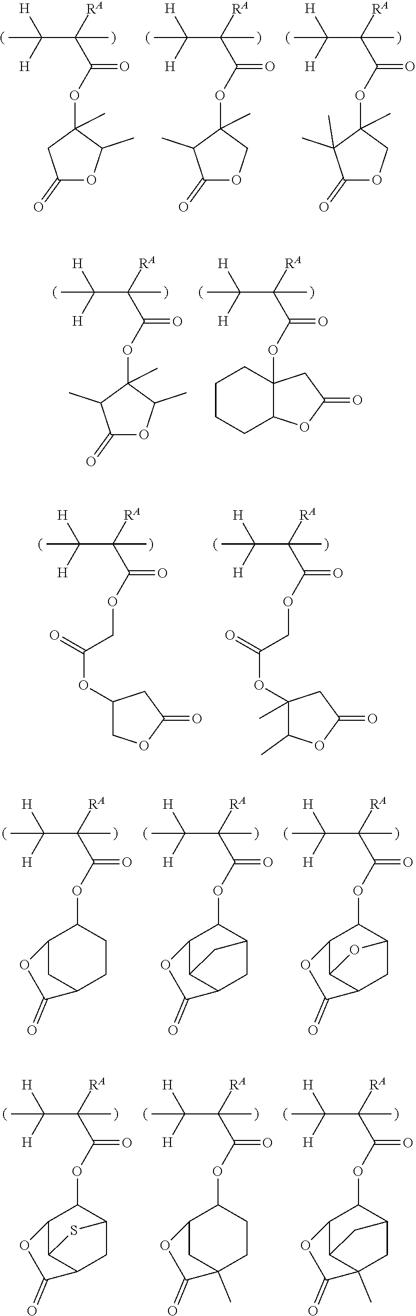

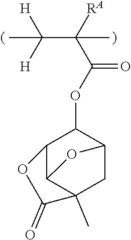

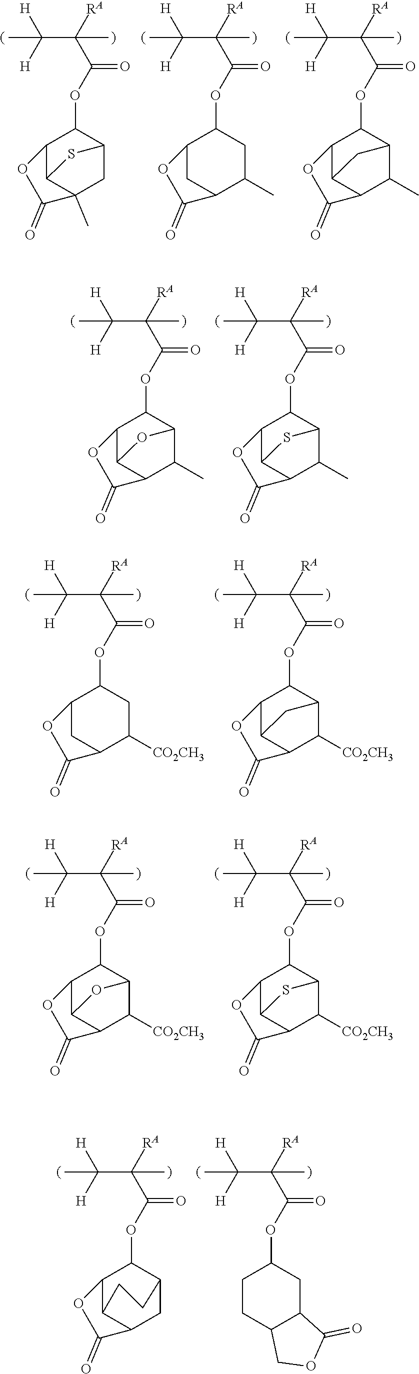

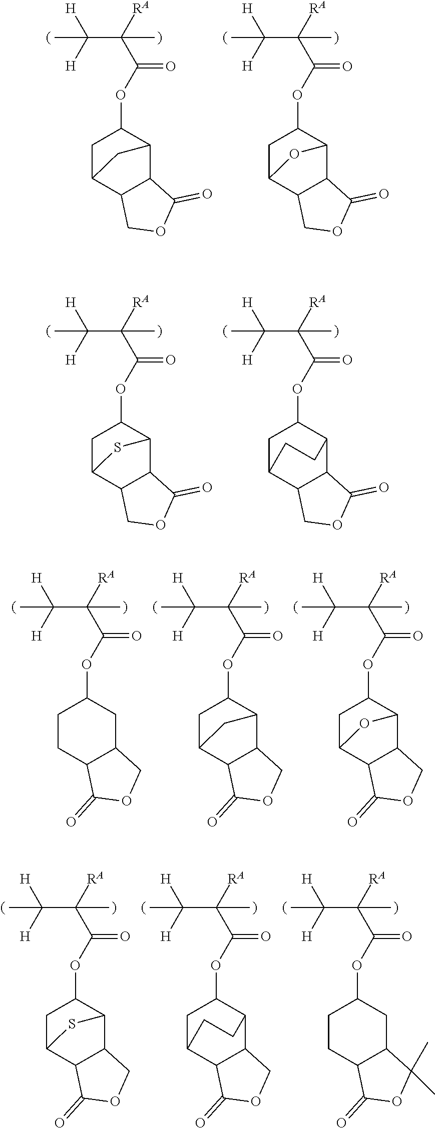

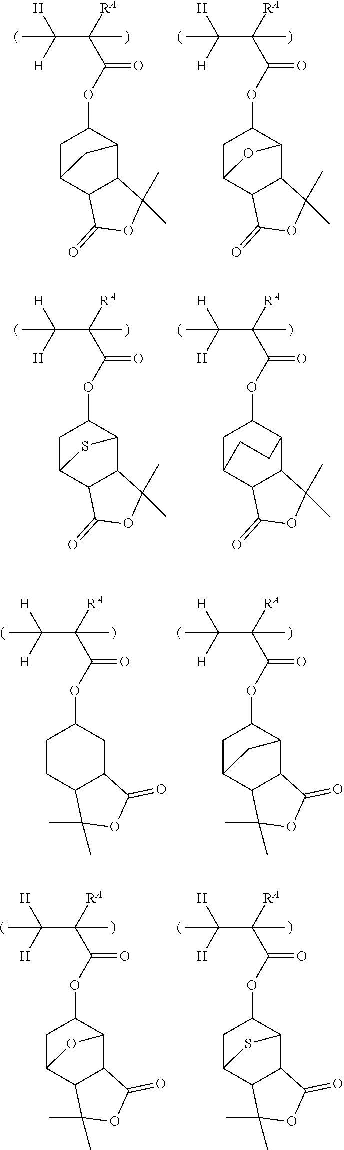

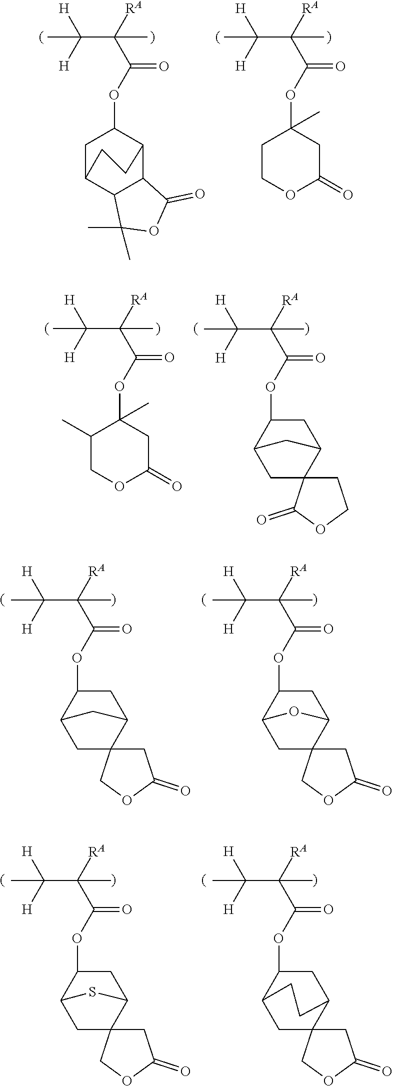

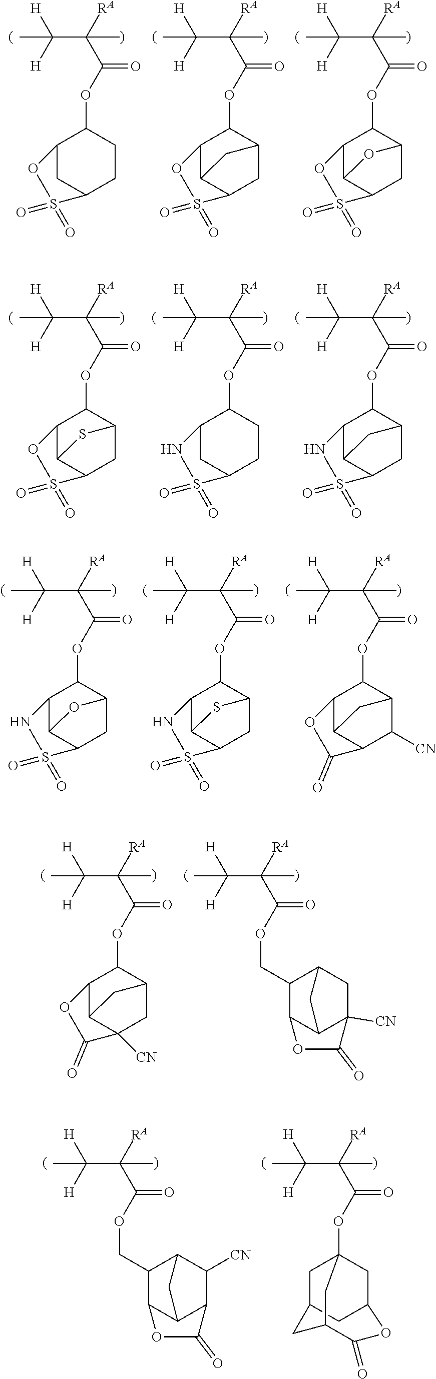

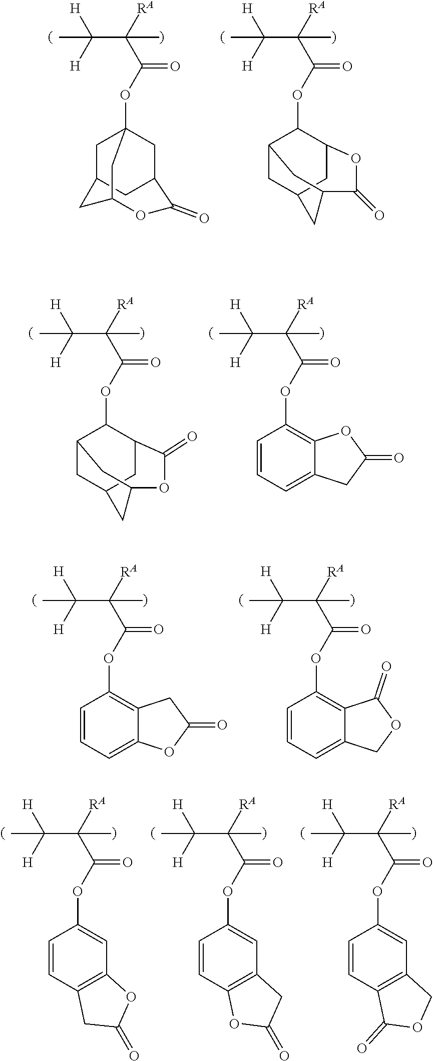

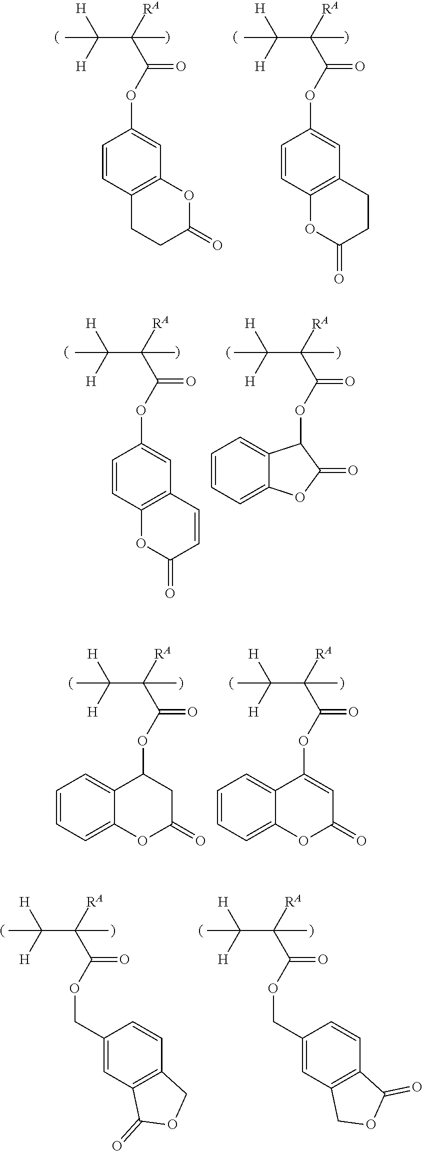

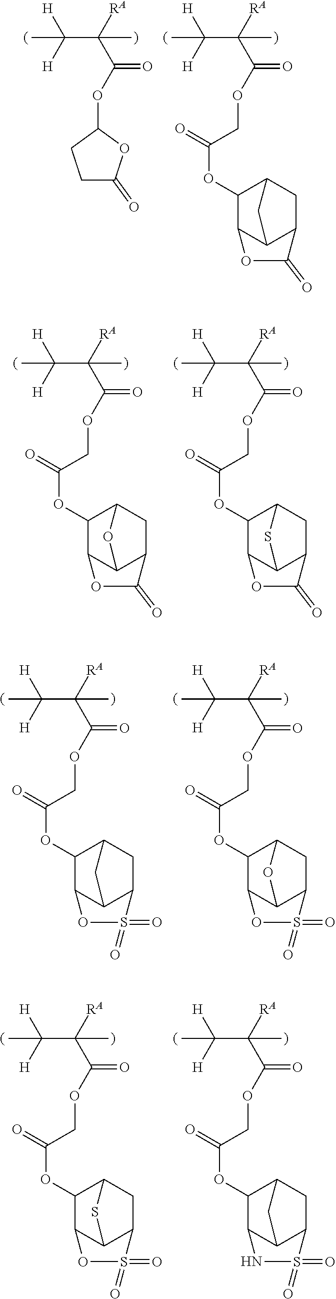

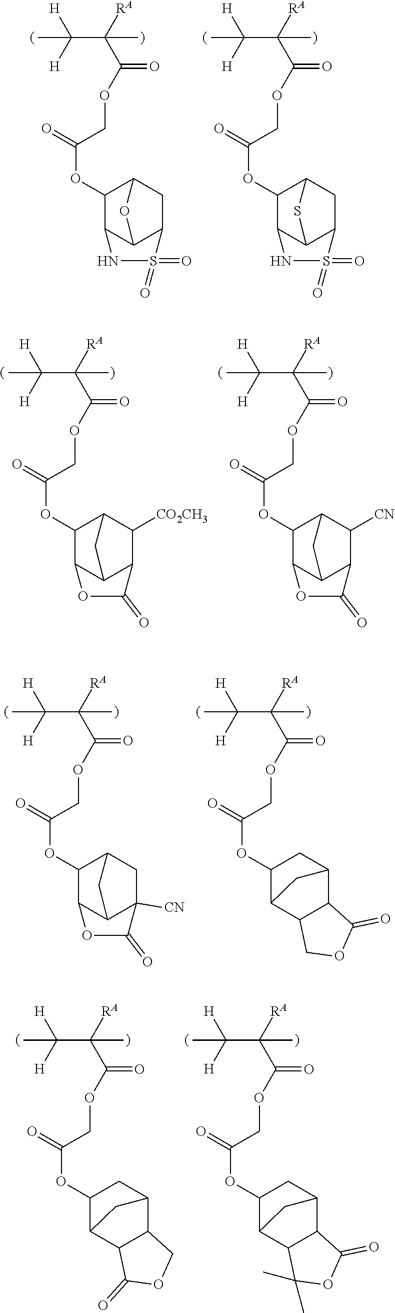

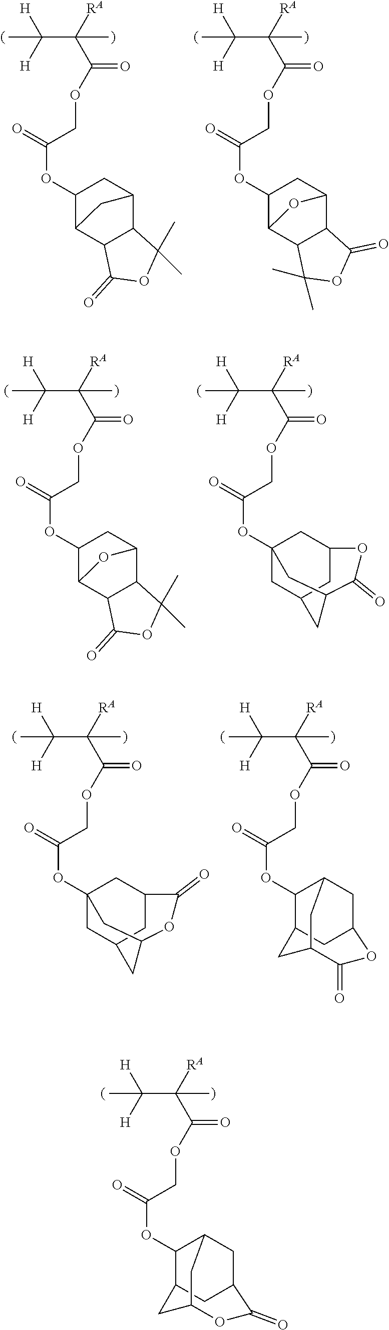

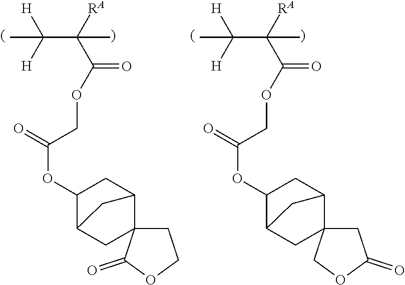

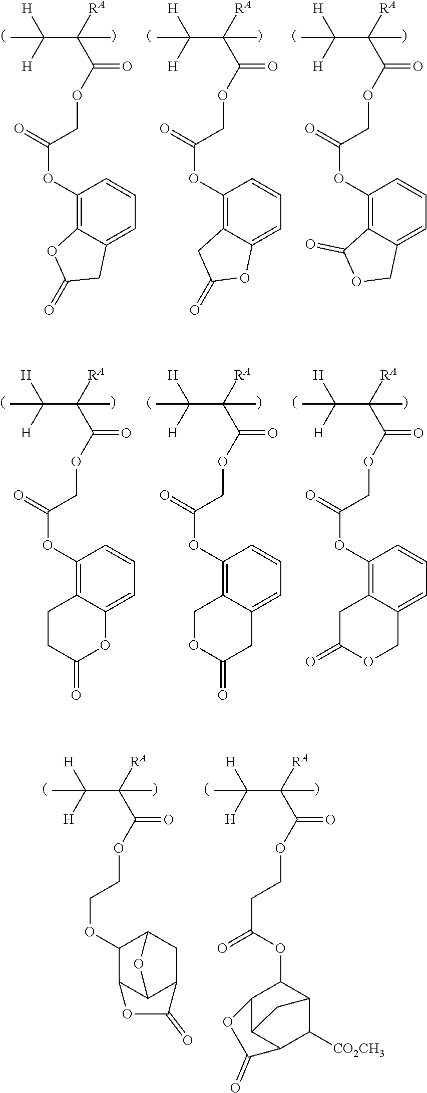

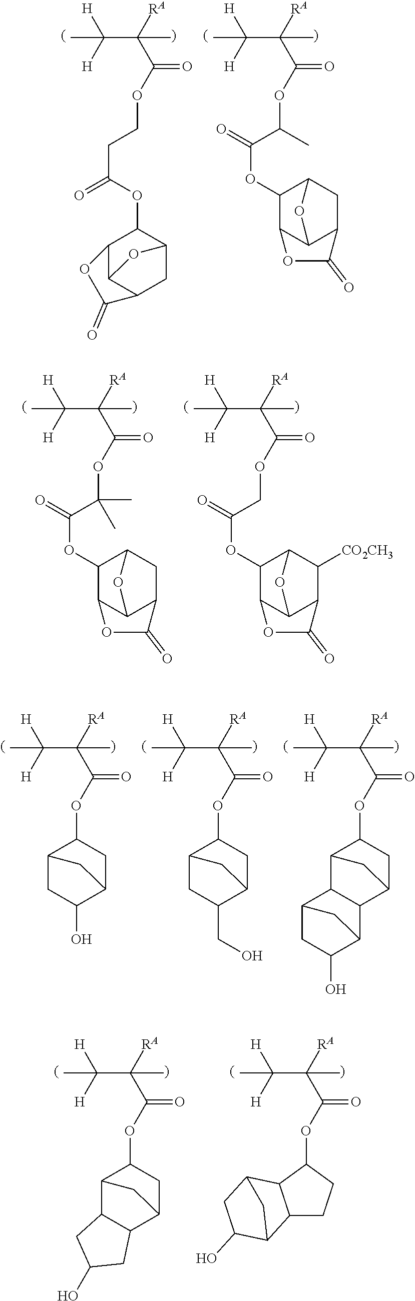

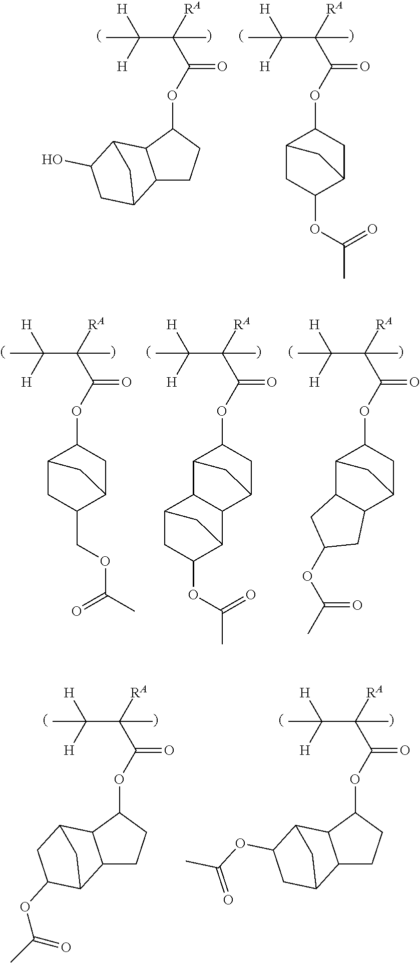

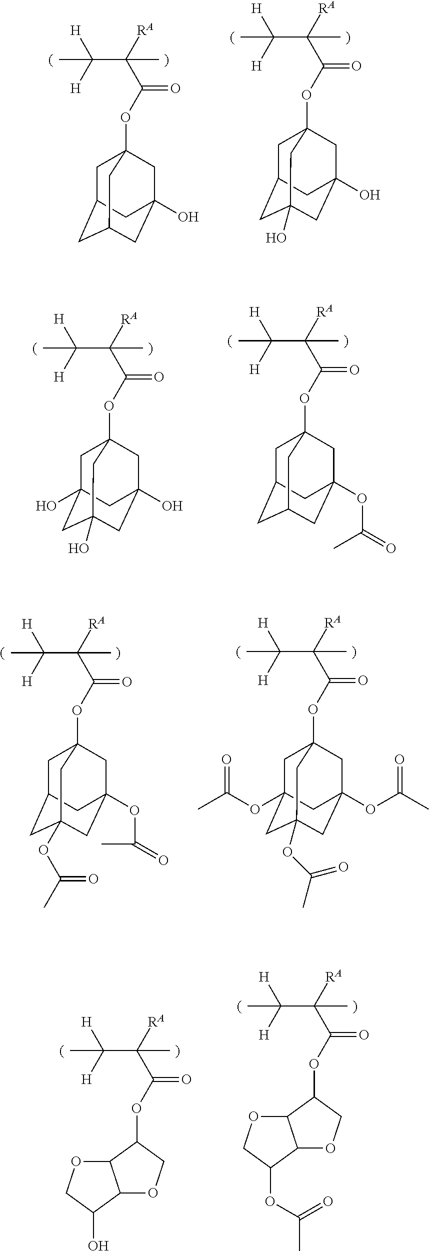

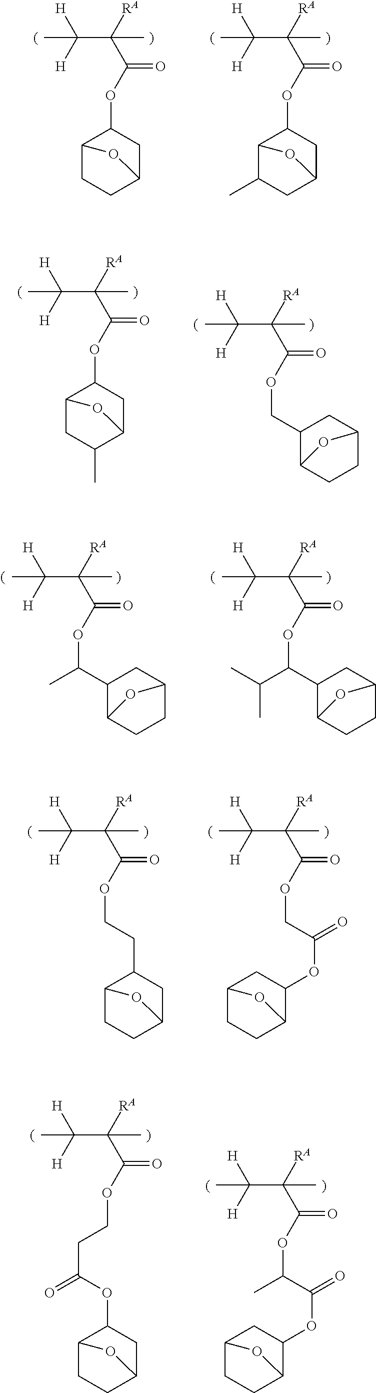

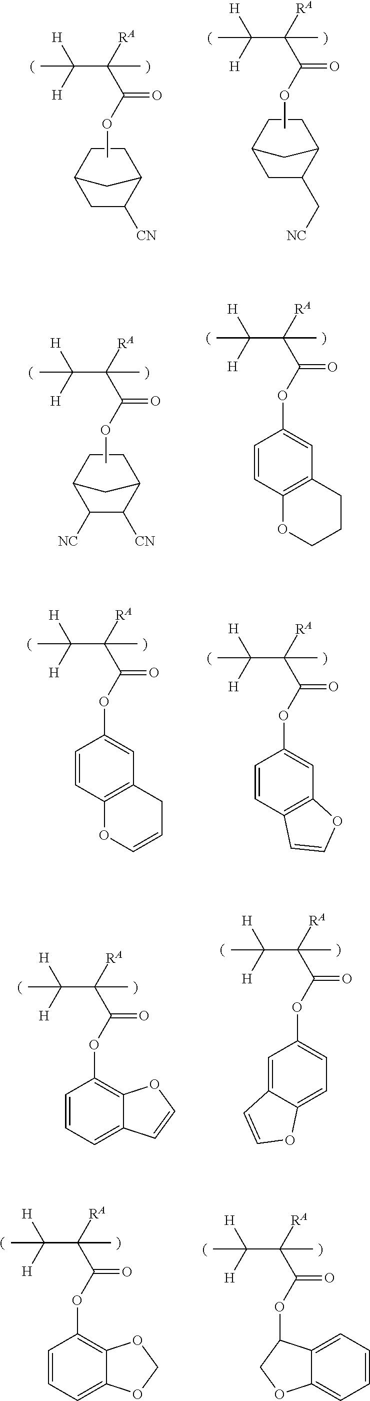

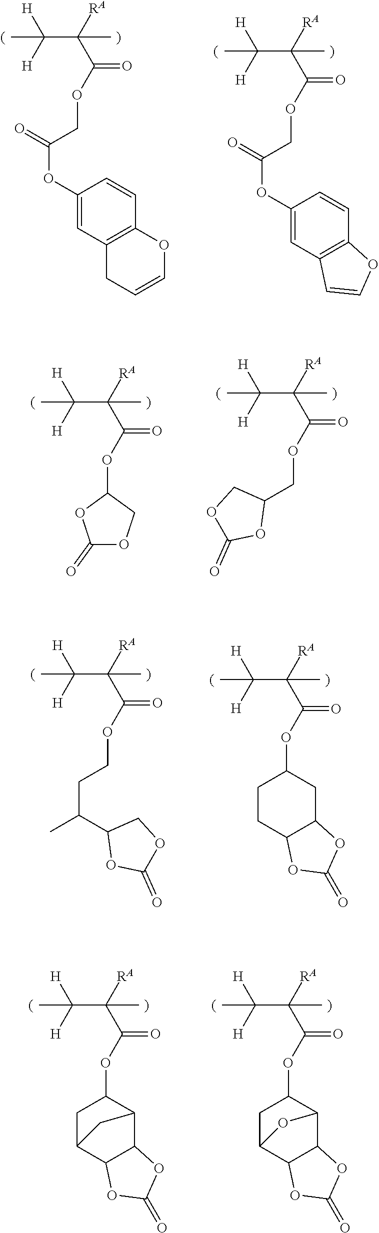

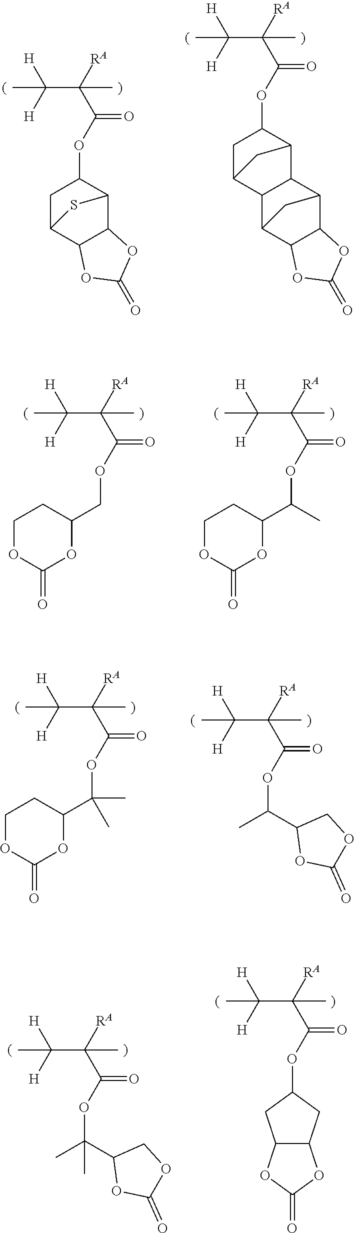

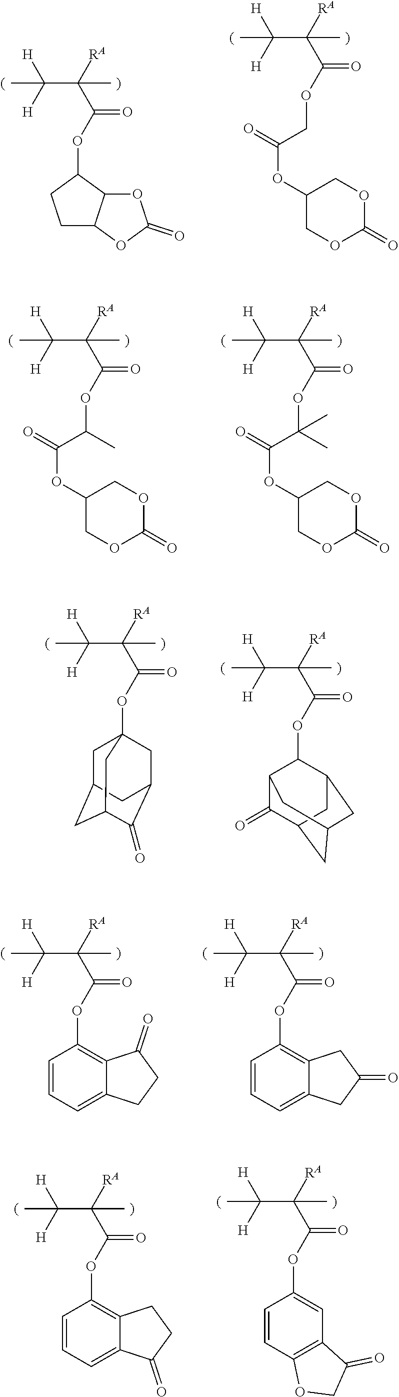

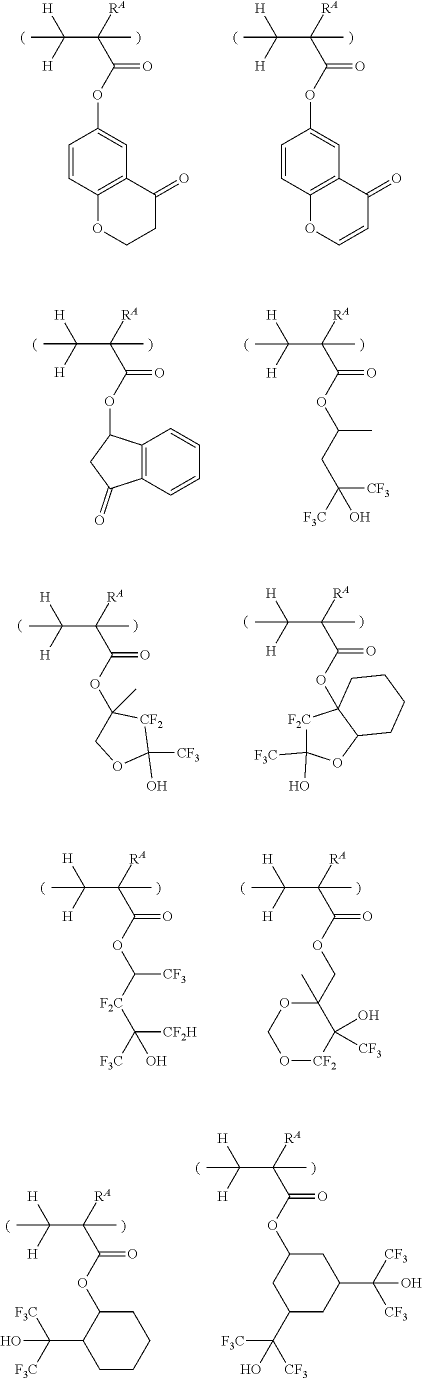

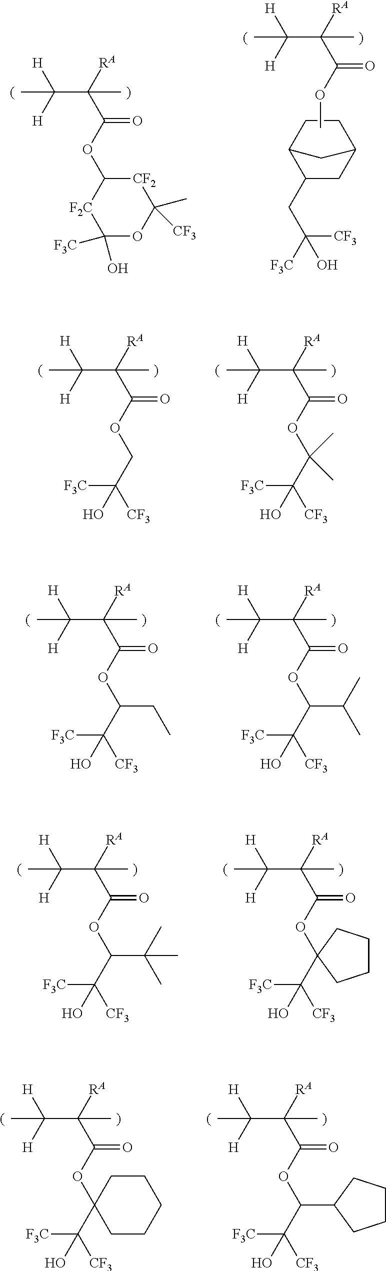

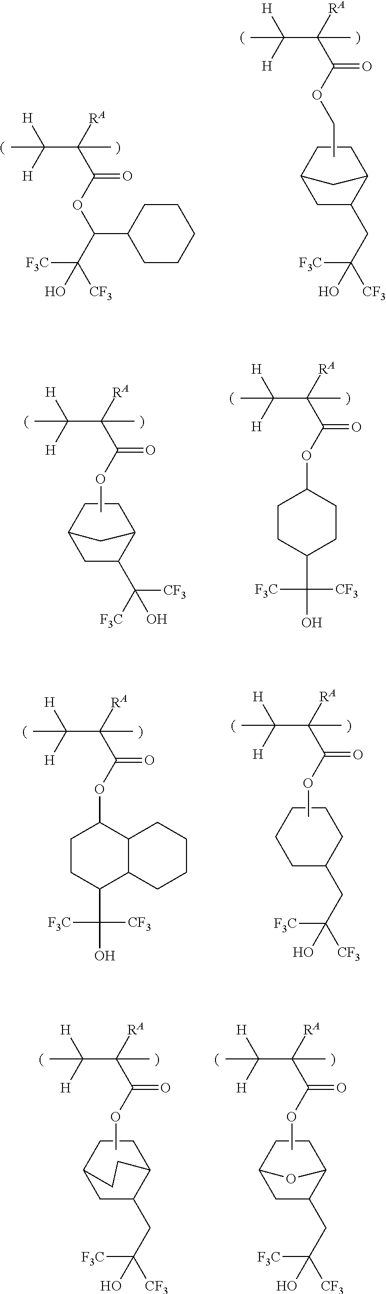

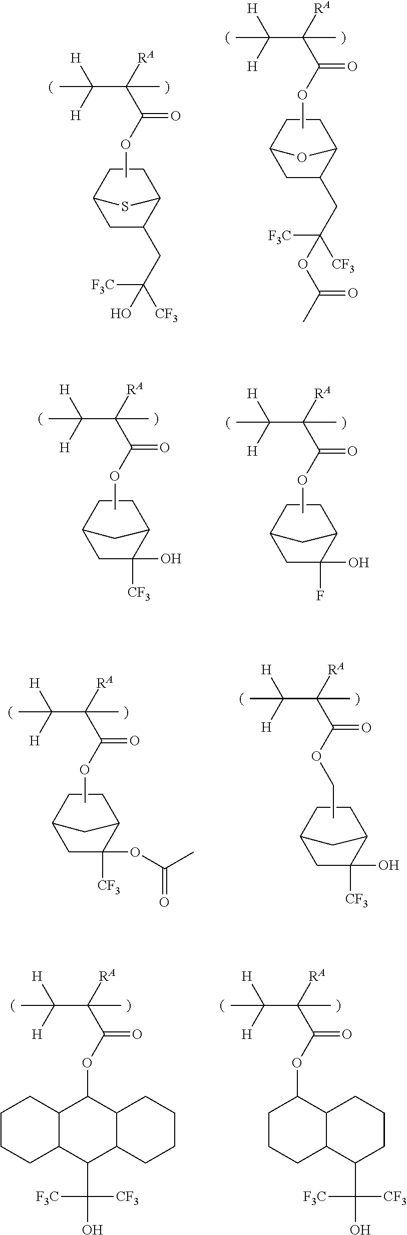

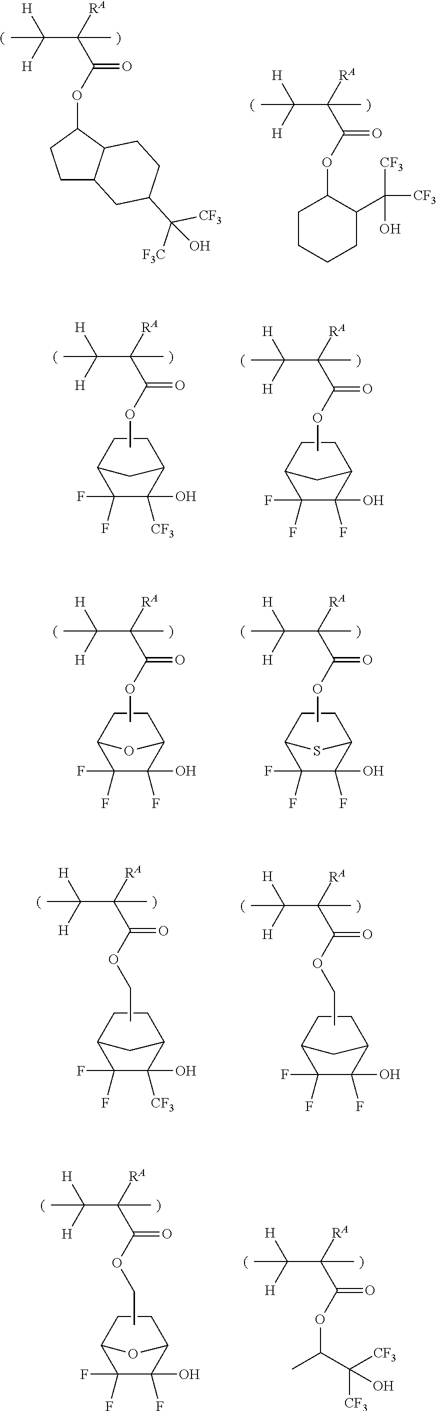

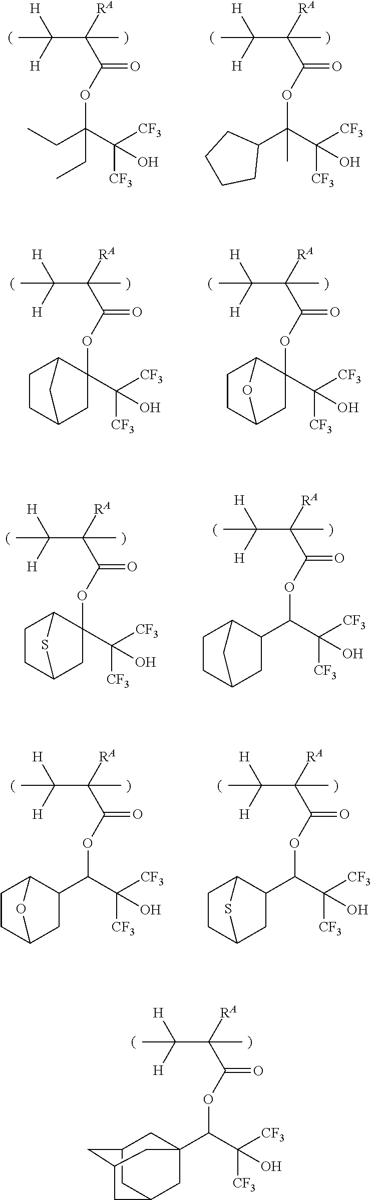

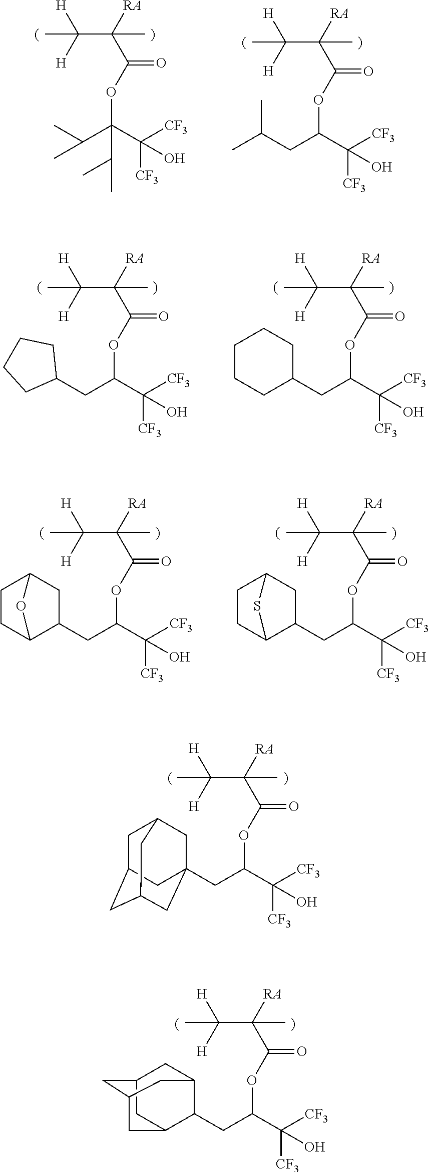

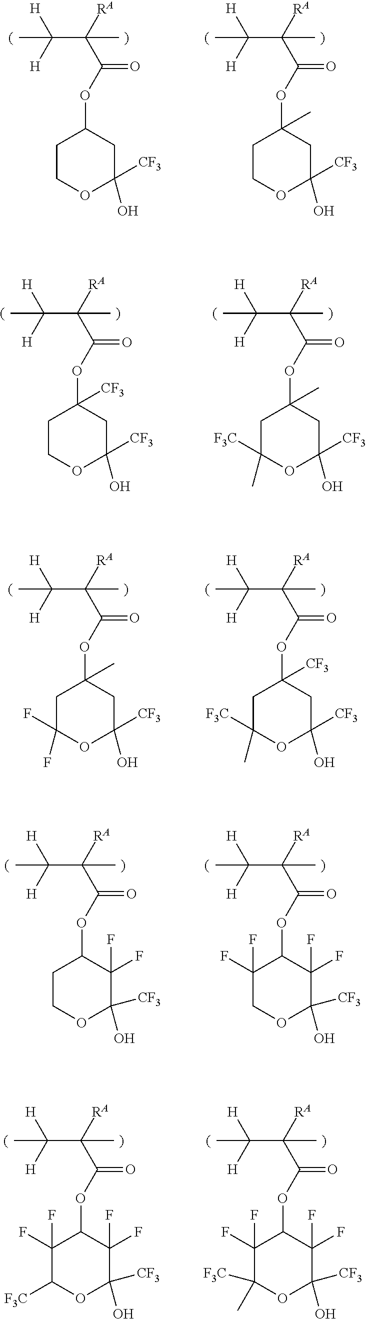

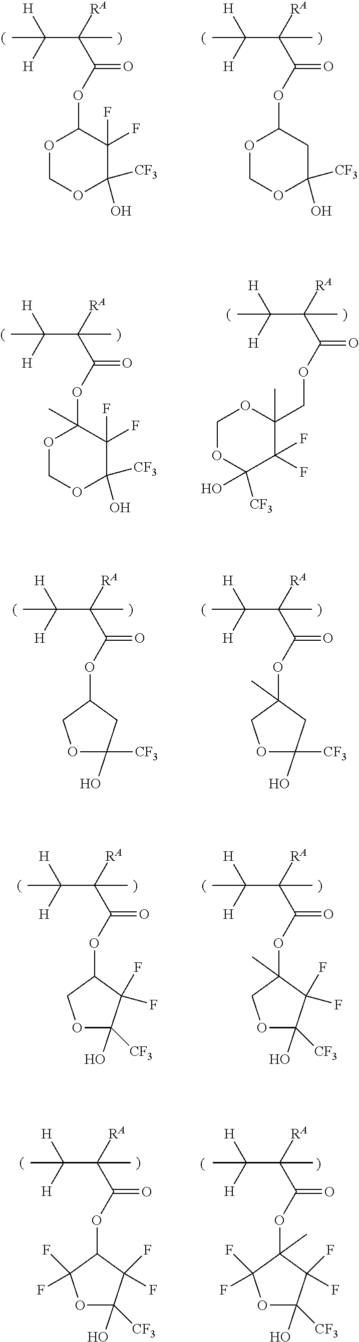

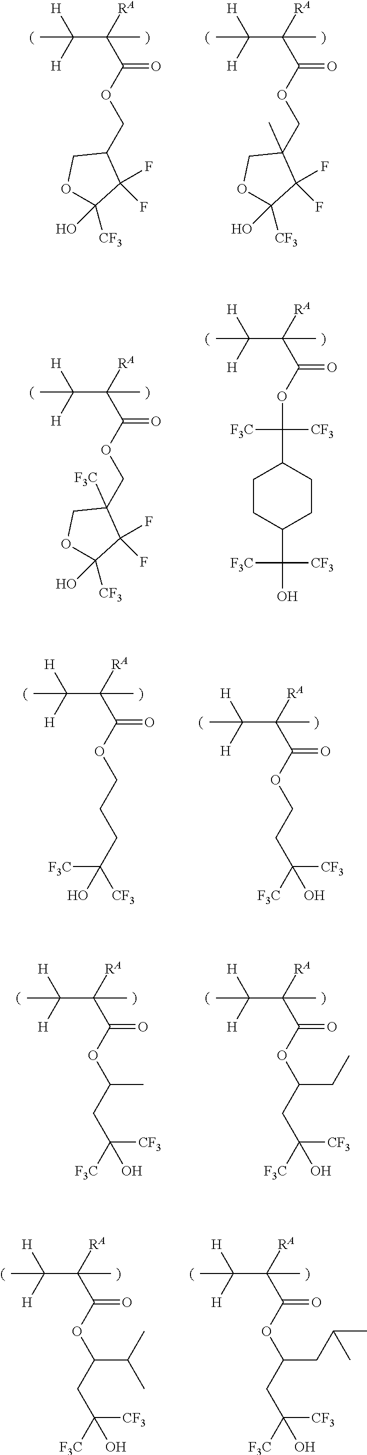

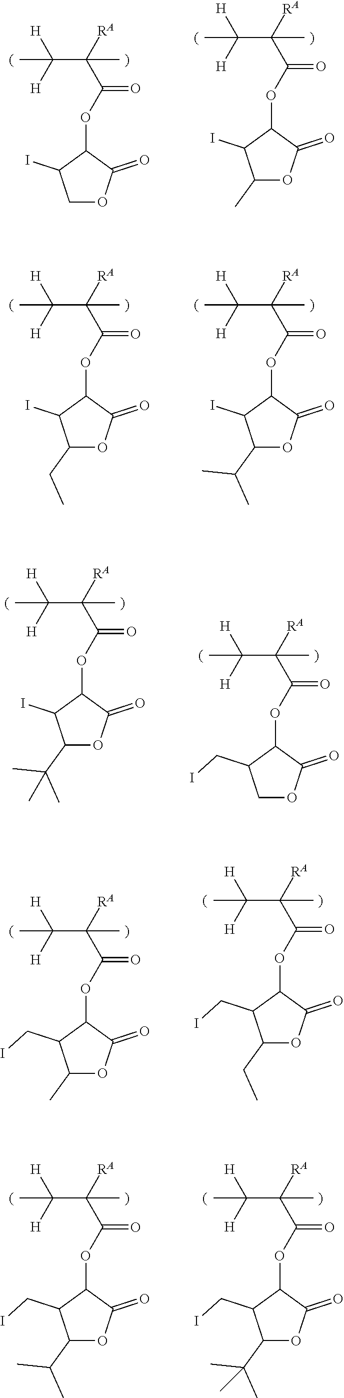

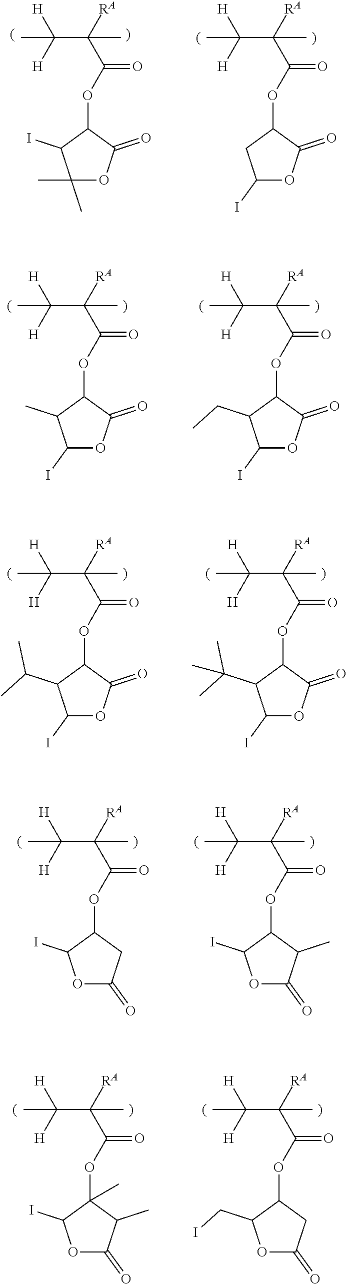

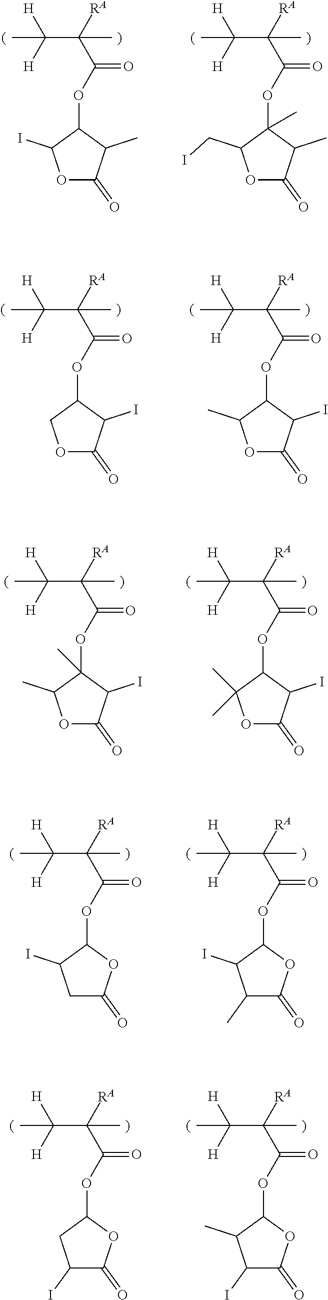

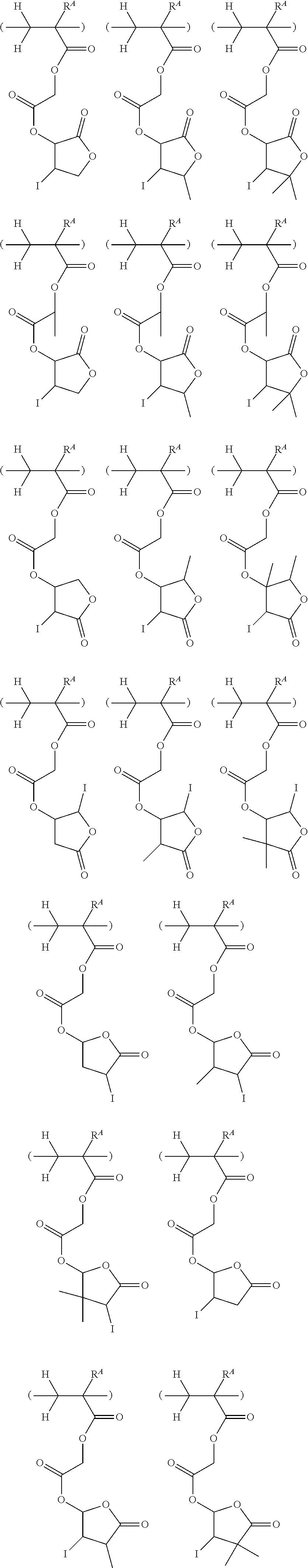

Repeating unit e preferably has a lactone ring as a polar group, in particular, for ArF lithography, and preferably has a phenolic site for KrF lithography, EB lithography, and EUV lithography.

The polymer may further contain a repeating unit having a structure in which hydroxy groups are each protected with an acid-unstable group (hereinafter, also referred to as repeating unit f). Repeating unit f is not limited as long as it has one or two or more structures each including a protected hydroxy group and the protective groups are decomposed by the action of an acid to produce hydroxy groups, but is preferably one represented by the following general formula (f):

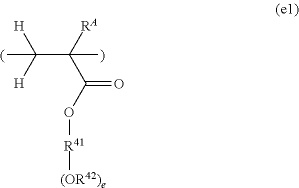

In the general formula (f), R^{A} is a hydrogen atom, a fluorine atom, a methyl group, or a trifluoromethyl group. R⁶¹ is an (e+1)-valent hydrocarbon group having 1 to 30 carbon atoms and optionally containing a heteroatom. R⁶² is an acid-unstable group. e is 1, 2, 3, or 4.

In the general formula (f), the acid-unstable group represented by R⁶² can be any one that deprotects by the action of an acid to generate a hydroxy group. The structure of R⁶² is not limited, but is preferably, for example, an acetal structure, a ketal structure, an alkoxycarbonyl group, or an alkoxymethyl group represented by the following general formula (f1), and particularly preferably an alkoxymethyl group represented by the following general formula (f1):

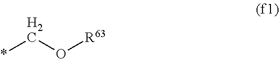

wherein * indicates bonding, and R⁶³ is a hydrocarbyl group having 1 to 15 carbon atoms.

Specific examples of the acid-unstable group represented by R⁶², the alkoxymethyl group represented by the general formula (f1), and repeating unit f include those described in JP 2020-111564 A and shown as examples in the description of repeating unit d.

The polymer may further contain repeating unit g derived from indene, benzofuran, benzothiophene, acenaphthylene, chromone, coumarin, norbornadiene, or a derivative of any of these. Specific examples of monomers that give repeating unit g include, but are not limited to, those shown in the following:

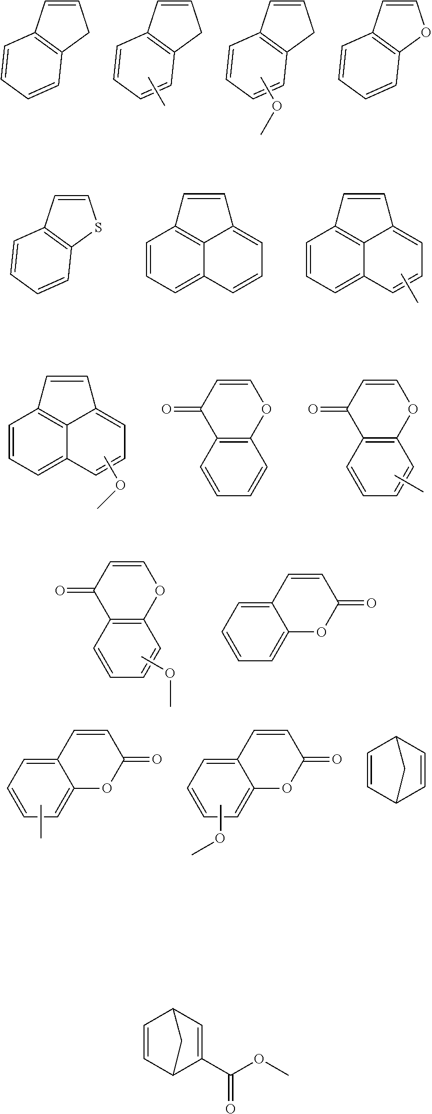

The base polymer may further contain repeating unit h derived from styrene, indan, vinylpyridine, or vinylcarbazole.

The content ratios of repeating units a, b1, b2, b3, c, d, e, f, g, and h in the polymer of the present invention are preferably 0<a≤0.4, 0≤b1≤0.8, 0≤b2≤0.8, 0≤b3≤0.6, 0<c≤0.6, 0≤d≤0.4, 0≤e≤0.6, 0≤f≤0.3, 0≤g≤0.3, and 0≤h≤0.3, and more preferably 0<a≤0.3, 0≤b1≤0.7, 0≤b2≤0.7, 0≤b3≤0.5, 0<c≤0.5, 0≤d≤0.3, 0≤e≤0.5, 0≤f≤0.2, 0≤g≤0.2, and 0≤h≤0.2. It should be noted that a+b1+b2+b3+c+d+e+f+g+h≤1.0 is satisfied.

The weight-average molecular weight (Mw) of the polymer is preferably 1000 to 500000, and more preferably 3000 to 100000. Mw in this range gives sufficient etching resistance, and does not cause the possibility of reduction in resolution due to failure in ensuring difference between the dissolution rates before and after exposure. In the present invention, Mw is a measurement in terms of polystyrene as determined by gel permeation chromatography (GPC) with use of tetrahydrofuran (THF) or N,N-dimethylformamide (DMF) as a solvent.

Moreover, the molecular weight distribution (Mw/Mn) of the polymer is preferably as narrow as 1.0 to 2.0 in order to obtain a resist composition that is suitably used for fine pattern dimensions because the influence of Mw/Mn tends to be more significant as pattern rules are finer. With Mw/Mn being in that range, less polymer having molecular weight lower or higher than Mw is present, and the possibility that an undesired matter is found on a pattern or a deteriorated pattern shape is formed is not caused after exposure.

Methods for synthesizing the polymer include a method of polymerizing a monomer that gives any of the repeating units described above by heating in an organic solvent with addition of a radical polymerization initiator.

Specific examples of the organic solvent to be used in the polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the initiator to be added is preferably 0.01 to 25 mol % based on the total of the monomers to be polymerized. The reaction temperature is preferably 50 to 150° C., and more preferably 60 to 100° C. The reaction time is preferably 2 to 24 hours, and, for production efficiency, the reaction time is more preferably 2 to 12 hours.

The polymerization initiator may be added to the monomer solution to feed to a reactor; alternatively, an initiator solution may be prepared separately from the monomer solution to feed them independently to a reactor. Radicals generated from the initiator during stand-by time may progress polymerization reaction to produce an ultra-high-molecular-weight product, and hence the monomer solution and the initiator solution are preferably each independently prepared and added dropwise for quality control. The acid-unstable group introduced to a monomer may be used as it is; otherwise, the acid-unstable group may be protected or partially protected after polymerization. A known chain transfer agent such as dodecylmercaptan and 2-mercaptoethanol may be used in combination for molecular weight adjustment. In this case, the amount of the chain transfer agent to be added is preferably 0.01 to 20 mol % based on the total of the monomers to be polymerized.

For monomers containing a hydroxy group, the hydroxy group may be replaced in advance with an acetal group in polymerization, which is readily deprotected with an acid such as an ethoxyethoxy group, and deprotected with a weak acid and water after polymerization, or replaced in advance with an acetyl group, a formyl group, a pivaloyl group, or the like and subjected to alkaline hydrolysis after polymerization.

For copolymerization of hydroxystyrene or hydroxyvinylnaphthalene, hydroxystyrene or hydroxyvinylnaphthalene and another monomer may be heat-polymerized in an organic solvent with addition of a radical polymerization initiator; alternatively, acetoxystyrene or acetoxyvinylnaphthalene may be used for deprotection of the acetoxy group through alkaline hydrolysis after polymerization to form polyhydroxystyrene or hydroxypolyvinylnaphthalene.

Specific examples of the base applicable in the alkaline hydrolysis include aqueous ammonia and triethylamine. The reaction temperature is preferably −20 to 100° C., and more preferably 0 to 60° C. The reaction time is preferably 0.2 to 100 hours, and more preferably 0.5 to 20 hours.

The amount of each monomer in the monomer solution can be appropriately set to give the aforementioned preferred content ratios of the repeating units, for example.

After obtaining the polymer by the production method, the reaction solution given through the polymerization reaction may be used as a final product, and alternatively a powder obtained by adding the polymerization solution to a poor solvent and performing a purification process to give a powder such as reprecipitation may be used as a final product; however, using a polymer solution obtained by dissolving a powder given through such a purification process in a solvent as a final product is preferred for work efficiency and quality stabilization.

Specific examples of the solvent to be used in this case include those shown in paragraphs [0144] to [0145] of JP 2008-111103 A, more specifically, ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; alcohols such as diacetone alcohol (DAA); high-boiling-point alcoholic solvents such as diethylene glycol, propylene glycol, glycerin, 1,4-butanediol, and 1,3-butanediol; and mixed solvents of any of these.

The polymer concentration of the polymer solution is preferably 0.01 to 30% by mass, and more preferably 0.1 to 20% by mass.

The reaction solution and polymer solution are preferably subjected to filter filtration. Undesired matters and gel that may cause defects can be removed through filter filtration, and this is effective with respect to quality stabilization.

Examples of the material of the filter to be used for the filter filtration include fluorocarbon, cellulose, nylon, polyester, and hydrocarbon materials; and in the filtration process for the chemically amplified resist composition, a filter formed of fluorocarbon material, what is called Teflon (registered trademark), hydrocarbon material such as polyethylene and polypropylene, or nylon is preferred. A pore size that fits with desired cleanliness can be appropriately selected for the filter, and the pore size is preferably 100 nm or less, and more preferably 20 nm or less. One of such filters may be used alone; alternatively, a plurality of such filters may be used in combination. For the filtration, the solution may be passed only once, but the filtration is preferably performed multiple times by circulating the solution. The filtration process can be performed any times in any order in the production process for the polymer, and it is preferred to filter the reaction solution after the polymerization reaction, the polymer solution, or both of them.

### [Chemically Amplified Resist Composition]

The present invention provides a chemically amplified resist composition containing (A) a base polymer containing the polymer described above.

### [Base Polymer (A)]

The chemically amplified resist composition of the present invention contains a base polymer containing the polymer described above as component (A).

For the polymer, one polymer may be used alone; alternatively, two or more polymers having different composition ratios, Mw, and/or Mw/Mn may be used in combination. Base polymer (A) may contain a hydrogen adduct of a ring-opening metathesis polymerization product in addition to the polymer, and those described in JP 2003-66612 A can be used therefor.

In the present invention, the chemically amplified resist composition preferably further contains one or more selected from (B) an organic solvent, (C) a quencher other than the sulfonium salt quencher, (D) an acid generator, and (E) a surfactant. The following describes the components in detail.

### [Organic Solvent (B)]

The chemically amplified resist composition of the present invention may contain an organic solvent as component (B). Organic solvent (B) may be any organic solvent capable of dissolving the components described above and components described later without limitation. Specific examples of such organic solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono tert-butyl ether acetate; lactones such as GBL; and mixed solvents of any of these.

Preferred among those organic solvents are 1-ethoxy-2-propanol, PGMEA, cyclohexanone, GBL, DAA, and mixed solvents of any of these, which are particularly superior in ability to dissolve the base polymer as component (A).

The amount of organic solvent (B) contained in the chemically amplified resist composition of the present invention is preferably 200 to 5000 parts by mass, and more preferably 400 to 3500 parts by mass per 80 parts by mass of base polymer (A). For organic solvent (B), one organic solvent may be used alone; alternatively, two or more organic solvents may be used as a mixture.

### [Additional Quencher (C)]

The chemically amplified resist composition of the present invention may contain a quencher other than the sulfonium salt quencher (hereinafter, also referred to as an additional quencher) as component (C). In the present invention, the term quencher refers to a material that prevents an acid generated from the photo-acid generator in the chemically amplified resist composition from diffusing to unexposed parts by trapping the acid for forming desired patterns.

Specific examples of additional quencher (C) include an onium salt represented by the following general formula (1) or (2):

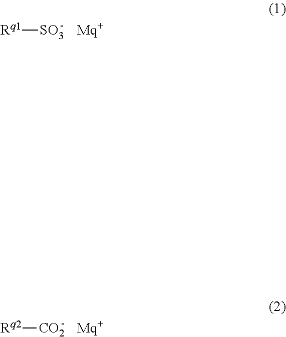

In the general formula (1), R^{q1} is a hydrogen atom or a hydrocarbyl group having 1 to 40 carbon atoms and optionally containing a heteroatom, provided that each hydrogen atom bound to the carbon atom at the a position of the sulfo group is not replaced with a fluorine atom or a fluoroalkyl group. In the general formula (2), R^{q2} is a hydrogen atom or a hydrocarbyl group having 1 to 40 carbon atoms and optionally containing a heteroatom.

Specific examples of the hydrocarbyl group represented by R^{q1}, having 1 to 40 carbon atoms, include an alkyl group having 1 to 40 carbon atoms such as a methyl group, an ethyl group, a n-propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, and a n-decyl group; a cyclic saturated hydrocarbyl group having 3 to 40 carbon atoms such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group; and an aryl group having 6 to 40 carbon atoms such as a phenyl group, a naphthyl group, and an anthracenyl group. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained.

Specific examples of the hydrocarbyl group represented by R^{q2} include not only the substituents shown as specific examples of R^{q1}, but also a fluorinated saturated hydrocarbyl group such as a trifluoromethyl group and a trifluoroethyl group, and a fluorinated aryl group such as a pentafluorophenyl group and a 4-trifluoromethylphenyl group.

Specific examples of the anion of the onium salt represented by the general formula (1) include, but are not limited to, those shown in the following:

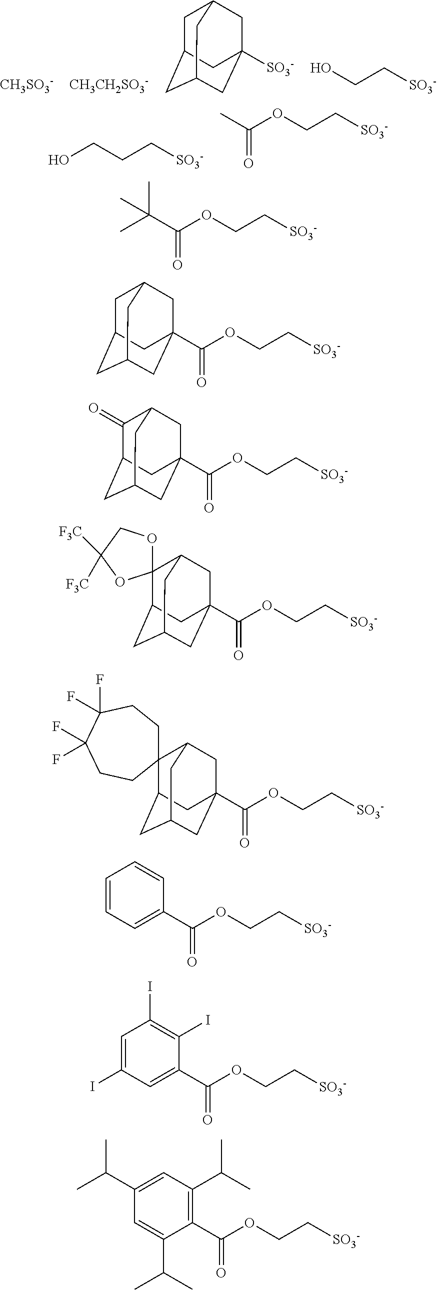

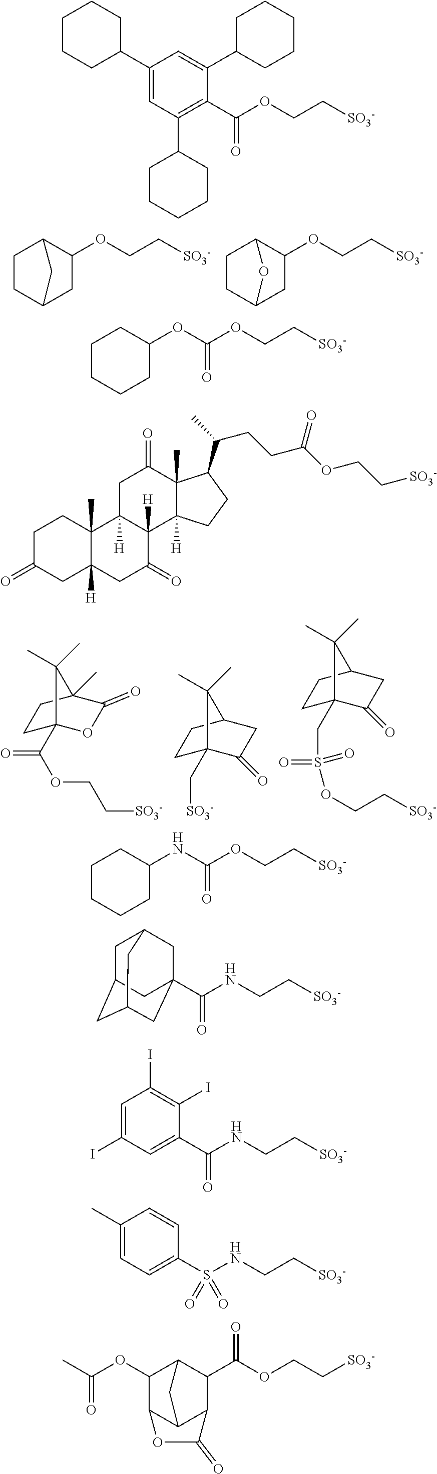

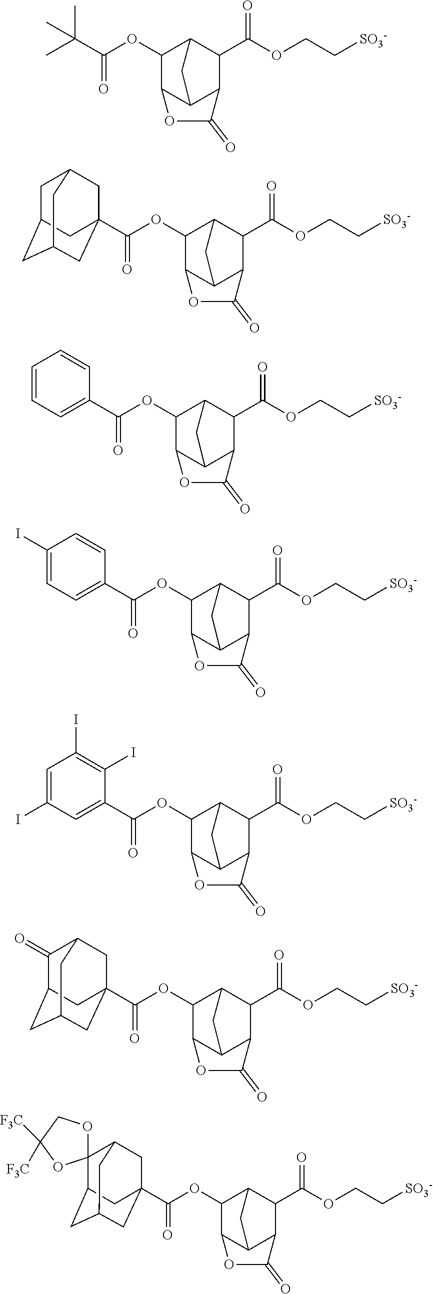

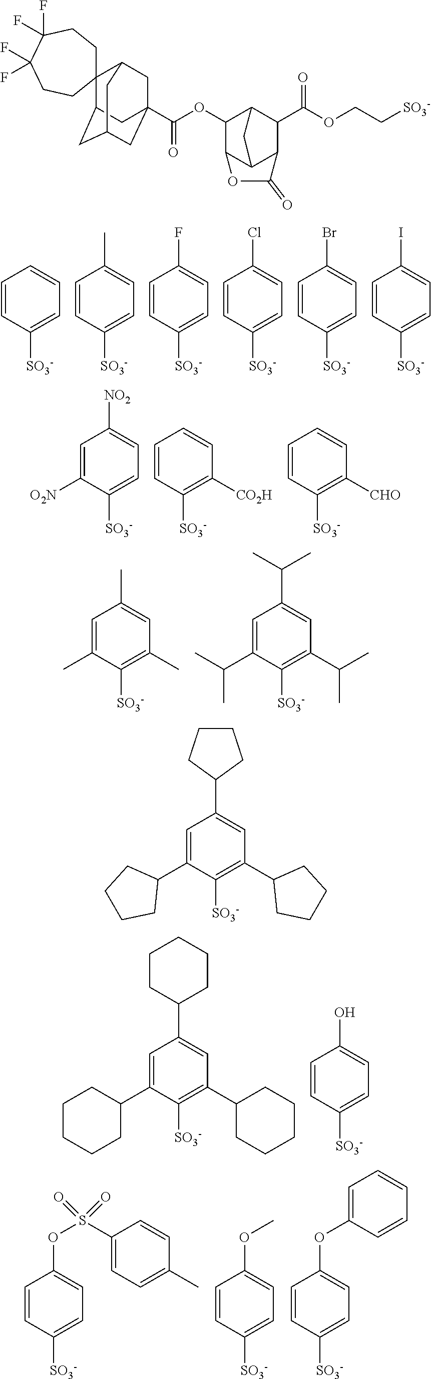

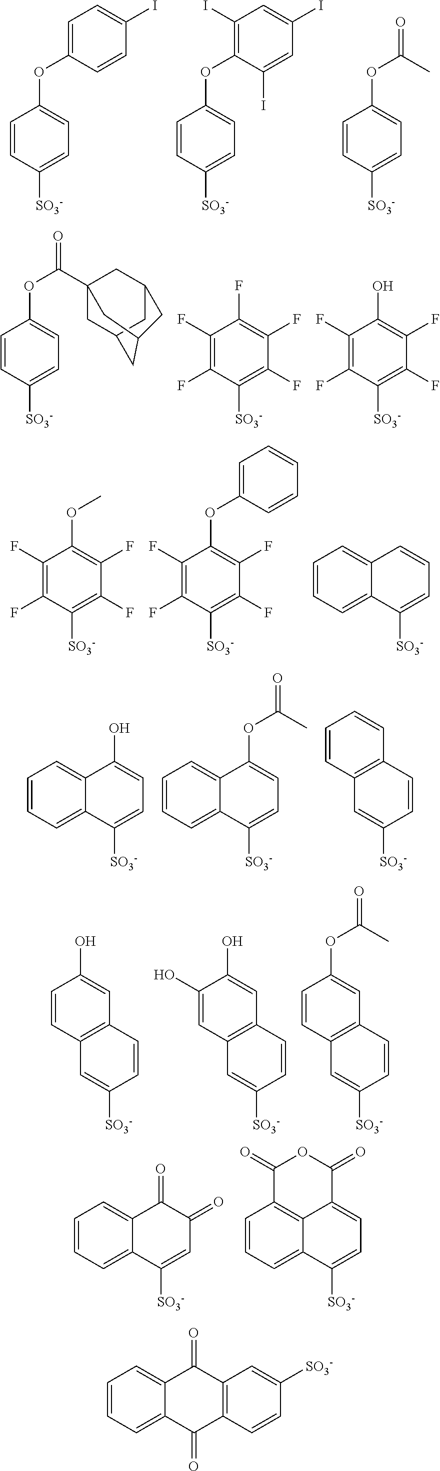

Specific examples of the anion of the onium salt represented by the general formula (2) include, but are not limited to, those shown in the following:

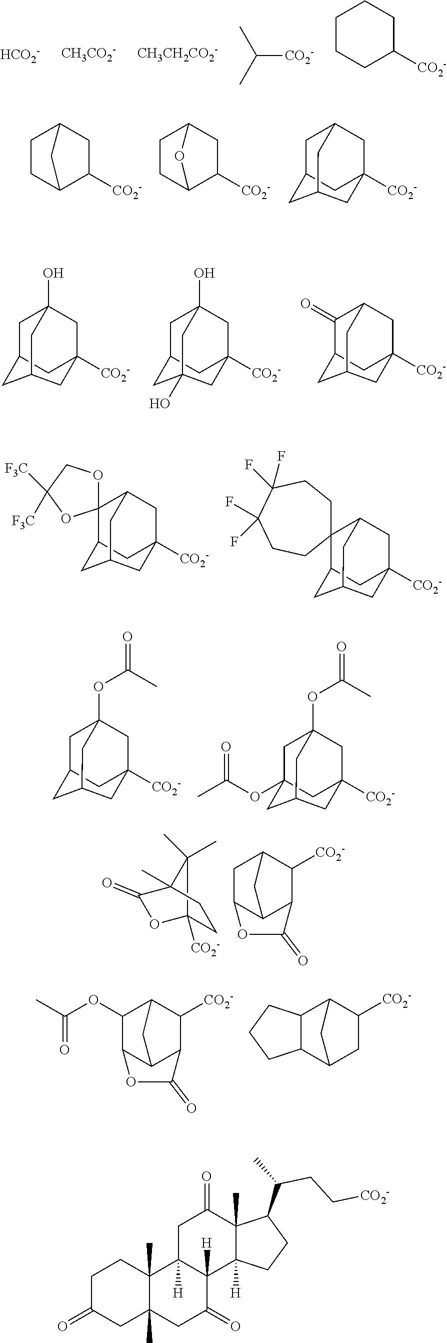

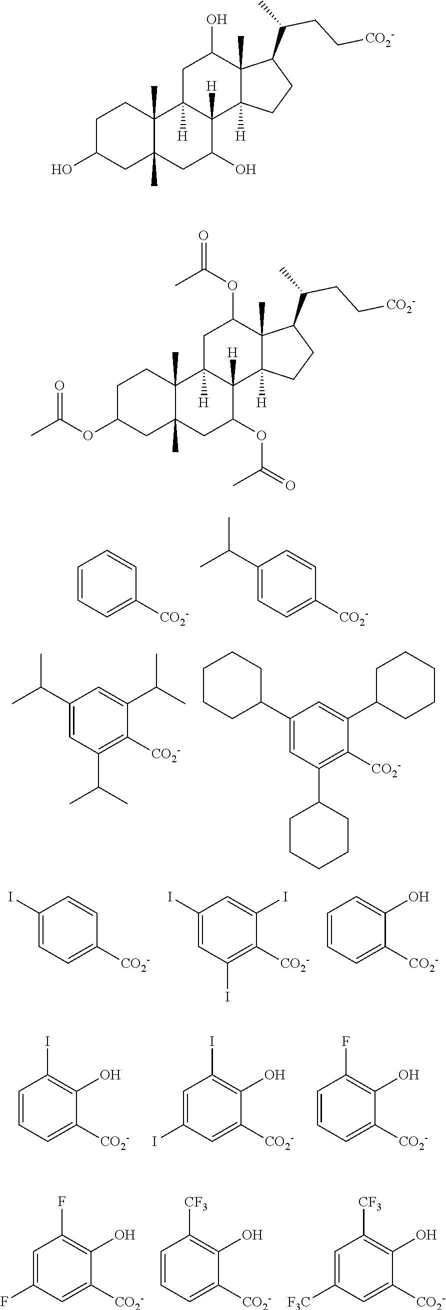

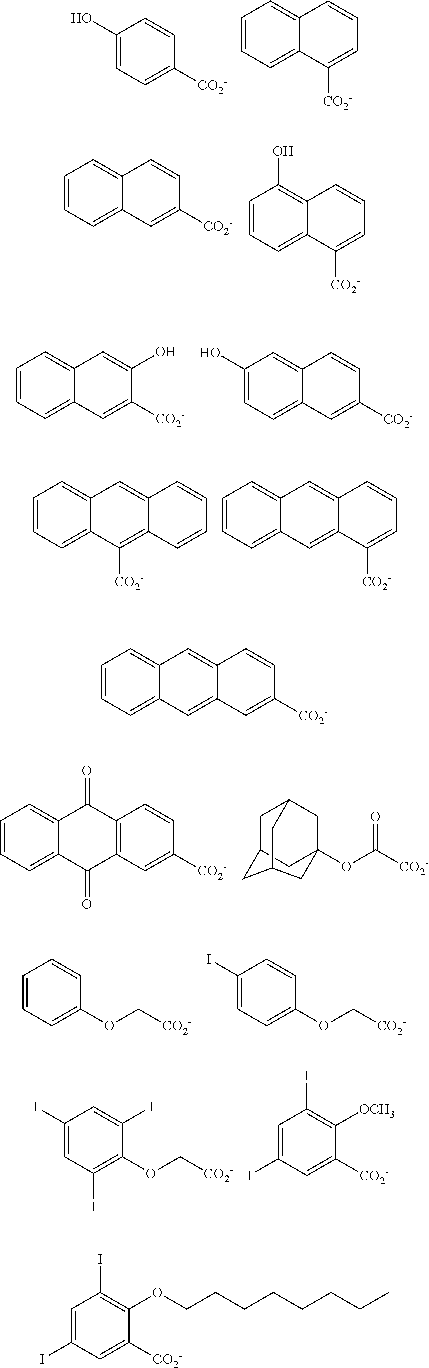

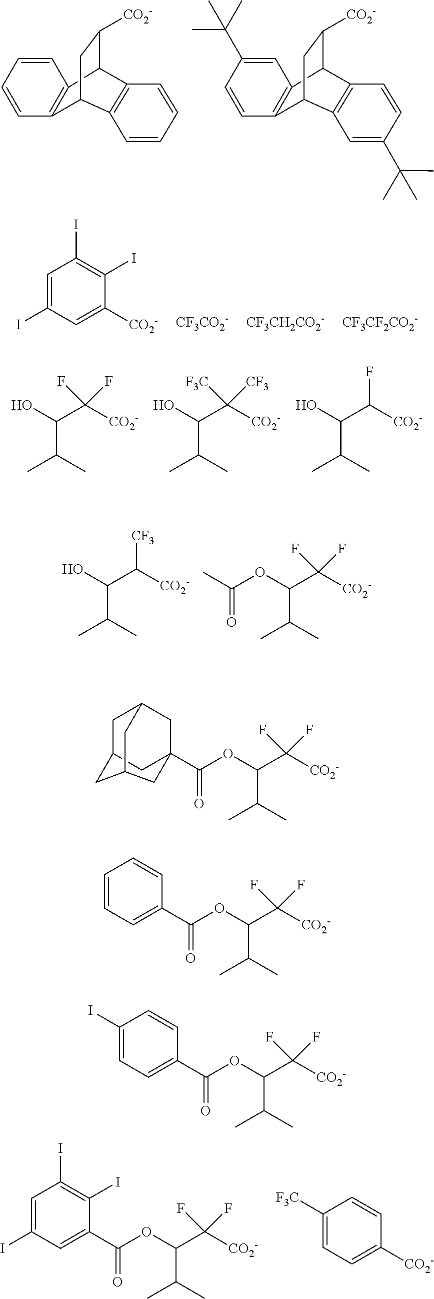

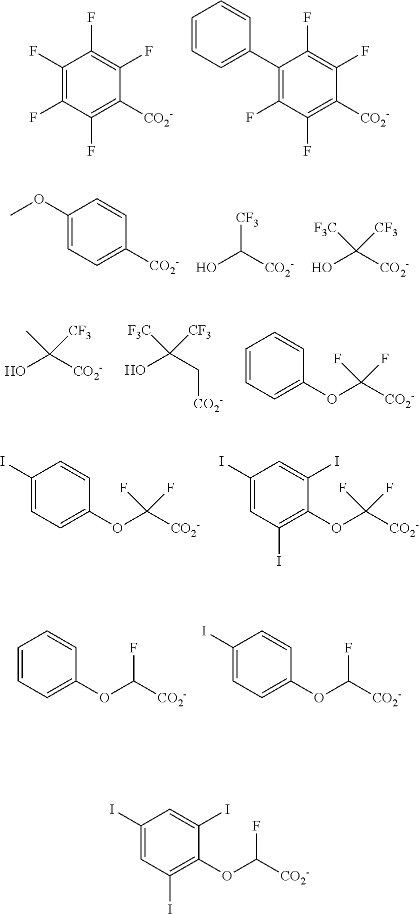

In the general formulae (1) and (2), Mq⁺ is an onium cation. The onium cation is preferably a sulfonium cation, an iodonium cation, or an ammonium cation. Specific examples of the sulfonium cation include, but are not limited to, those shown as examples of the sulfonium cation of the sulfonium salt monomer represented by the general formula (a), those shown in paragraphs [0102] to [0125] of JP 2024-3744 A, those shown in paragraphs [0044] to [0049] of WO2024/128017 A1, those shown inparagraphs [0035] to [0046] of JP 7491173 B, and that represented by the general formula (d3). Specific examples of the iodonium cation include, but are not limited to, those shown in paragraph [0181] of JP 2024-000259 A. The ammonium cation is preferably that represented by the following general formula (am-1), but is not limited thereto:

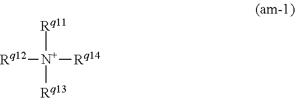

In the general formula (am-1), R^{q11} to R^{q14} are each independently a hydrocarbyl group having 1 to 40 carbon atoms and optionally containing a heteroatom. R^{q11} and R^{q12} are optionally bound together to form a ring together with the nitrogen atom to which they are bound. Specific examples of the hydrocarbyl group include those shown as examples of the hydrocarbyl group represented by R² in the description of the general formula (a).

Specific examples of the ammonium cation represented by the general formula (am-1) include, but are not limited to, those shown in the following:

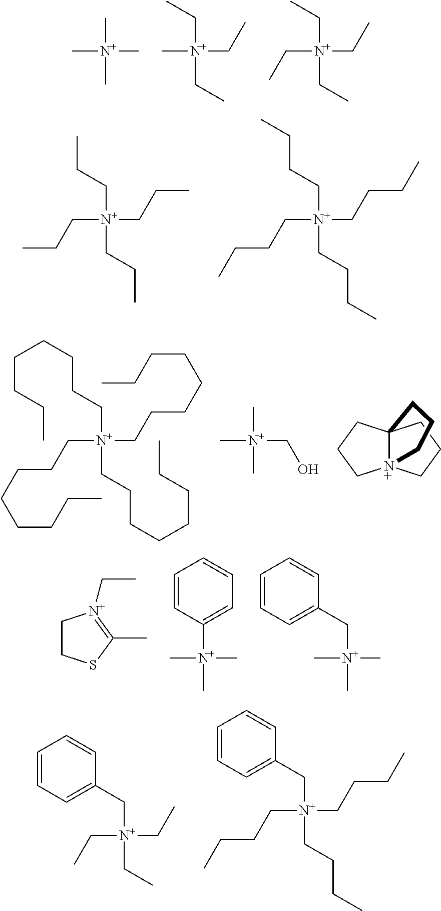

Specific examples of the onium salt represented by the general formula (1) or (2) include any combinations of the aforementioned anions and cations. These onium salts are easily prepared through ion exchange reaction by using a known organic chemistry method. For the ion exchange reaction, for example, reference can be made to JP 2007-145797 A.

The onium salt represented by the general formula (1) or (2) acts as a quencher in the chemically amplified resist composition of the present invention. This is due to the fact that each counteranion of the onium salt is a conjugate base of a weak acid. The term weak acid mentioned here refers to an acid that exhibits acidity such that the acid-unstable group of each acid-unstable group-containing unit used for the base polymer cannot be deprotected. The onium salt represented by the general formula (1) or (2) acts as a quencher when used in combination with an onium salt photo-acid generator having, as a counteranion, a conjugate base of a strong acid such as sulfonic acid fluorinated at the a position. Specifically, if an onium salt that generates a strong acid such as sulfonic acid fluorinated at the a position and an onium salt that generates a weak acid such as sulfonic acid not fluorinated and carboxylic acid are used as a mixture, a strong acid generated from a photo-acid generator through high-energy ray irradiation collides with the onium salt having an unreacted weak acid anion, and then the weak acid is released as a result of salt exchange and an onium salt having a strong acid anion is formed. Through this process, the strong acid is exchanged with the weak acid, which has lower catalytic ability, and hence the acid is apparently deactivated; thus, acid diffusion can be controlled.

Applicable as quencher (C) are an onium salt described in JP 6848776 B, which has sulfonium cation and phenoxide anion moieties in the same molecule, an onium salt described in JP 6583136 B and JP 2020-200311 A, which has sulfonium cation and carboxylate anion moieties in the same molecule, and an onium salt described in JP 6274755 B, which has iodonium cation and carboxylate anion moieties in the same molecule.

Here, if the phot-acid generator that generates a strong acid is an onium salt, the strong acid generated through high-energy ray irradiation can be exchanged with a weak acid as described above, but the weak acid generated through high-energy ray irradiation is expected to be less likely to undergo salt exchange through collision with the onium salt that generates an unreacted strong acid. This is due to the phenomenon that the onium cation readily forms an ion pair with a stronger acid anion.

If the chemically amplified resist composition of the present invention contains the onium salt represented by the general formula (1) or (2) as quencher (C), the amount of quencher (C) contained therein is preferably 0.1 to 20 parts by mass, and more preferably 0.1 to 10 parts by mass per 80 parts by mass of base polymer (A). The case that the amount of the onium salt quencher contained as component (C) falls within that range is preferred because good resolution results and remarkable reduction in sensitivity is not caused. For the onium salt represented by the general formula (1) or (2), one onium salt may be used alone; alternatively, two or more onium salts may be used in combination.

The chemically amplified resist composition of the present invention may contain a nitrogen-containing compound as additional quencher (C). Specific examples of the nitrogen-containing compound as component (C) include any of primary, secondary, and tertiary amine compounds shown in paragraphs [0146] to [0164] of JP 2008-111103 A, especially, an amine compound having a hydroxy group, an ether bond, an ester bond, a lactone ring, a cyano group, or a sulfonate bond. In addition, a compound obtained by protecting a primary or secondary amine described in JP 3790649 B with a carbamate group can be shown as an example.

A sulfonic acid sulfonium salt having a nitrogen-containing substituent may be used as the nitrogen-containing compound. Such a compound functions as a quencher in unexposed parts, and in exposed parts loses the quencher ability through neutralization with an acid generated from the compound itself, in other words, functions as a photodegradable base. Use of a photodegradable base advantageously gives stronger contrast between exposed parts and unexposed parts. For the photodegradable base, for example, reference can be made to JP 2009-109595 A and JP 2012-46501 A.

If the chemically amplified resist composition of the present invention contains the nitrogen-containing compound as additional quencher (C), the amount of the nitrogen-containing compound contained therein is preferably 0.001 to 12 parts by mass, and more preferably 0.01 to 8 parts by mass per 80 parts by mass of base polymer (A). For the nitrogen-containing compound, one nitrogen-containing compound may be used alone; alternatively, two or more nitrogen-containing compounds may be used in combination.

### [Acid Generator (D)]

The chemically amplified resist composition of the present invention may contain acid generator (D) according to the purpose of the present invention. Examples of the acid generator (D) include a compound that generates an acid in response to an active ray or radiation (photo-acid generator). The photo-acid generator may be any compound that generates an acid through high-energy ray irradiation without limitation, and is preferably a compound that generates sulfonic acid, imidic acid, or methidic acid. Preferred photo-acid generators include sulfonium salt, iodonium salt, sulfonyldiazomethane, N-sulfonyloxyimide, and oxime-O-sulfonate acid generators. Specific examples of the acid generator include those shown in paragraphs [0122] to [0142] of JP 2008-111103 A.

For the photo-acid generator, a sulfonium salt represented by the following general formula (3-1) and an iodonium salt represented by the following general formula (3-2) can also be suitably used:

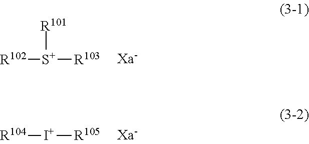

In the general formulae (3-1) and (3-2), R¹⁰¹ to R¹⁰⁵ are each independently a halogen atom or a hydrocarbyl group having 1 to 20 carbon atoms and optionally containing a heteroatom. Specific examples of the halogen atom and hydrocarbyl group include those shown as examples of the halogen atom and hydrocarbyl group represented by R² in the description of the general formula (a). Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a nitro group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained. R¹⁰¹ and R¹⁰² are optionally bound together to form a ring together with the sulfur atom to which they are bound. Specific examples of the ring formed in this case include those shown as examples of the ring that can be formed through bonding of any two of the three substituents bound to S⁺ together with the sulfur atom to which the two substituents are bound in the description of the general formula (a).

Specific examples of the cation of the sulfonium salt represented by the general formula (3-1) include, but are not limited to, those shown as examples of the sulfonium cation of the sulfonium salt monomer represented by the general formula (a), those shown in paragraphs [0102] to [0125] of JP 2024-3744 A, those shown in paragraphs [0044] to [0049] of WO 2024/128017 A1, those shown in paragraphs [0035] to [0046] of JP 7491173 B, and that represented by the formula (d3). Specific examples of the cation of the iodonium salt represented by the formula (3-2) include, but are not limited to, those shown in paragraph [0181] of JP 2024-000259 A.

In the general formulae (3-1) and (3-2), Xa⁻ is an anion selected from the following general formulae (3A) to (3D):

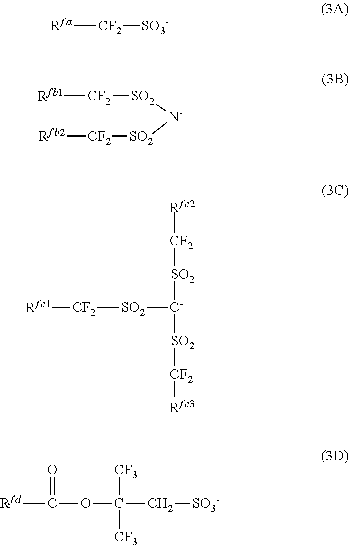

In the general formula (3A), R^{fa} is a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those to be shown as examples later in description of R^{fa1} in general formula (3A′).

The anion represented by the formula (3A) is preferably one represented the following general formula (3A′):

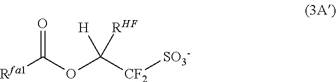

In the general formula (3A′), R^{H}F is a hydrogen atom or a trifluoromethyl group, and preferably a trifluoromethyl group.

In the general formula (3A′), R^{fa1} is a hydrocarbyl group having 1 to 38 carbon atoms and optionally containing a heteroatom. For example, the heteroatom is preferably an oxygen atom, a nitrogen atom, a sulfur atom, or a halogen atom, and more preferably an oxygen atom. The hydrocarbyl group is particularly preferably one having 6 to 30 carbon atoms for achievement of high resolution in fine patterning.

The hydrocarbyl group represented by R^{fa1}, having 1 to 38 carbon atoms, may be saturated or unsaturated, and may be linear, branched, or cyclic. Specific examples thereof include an alkyl group having 1 to 38 carbon atoms such as a methyl group, an ethyl group, a n-propyl group, an isopropyl group, a n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; a cyclic saturated hydrocarbyl group having 3 to 38 carbon atoms such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; an unsaturated aliphatic hydrocarbyl group having 2 to 38 carbon atoms such as an allyl group and a 3-cyclohexenyl group; an aryl group having 6 to 38 carbon atoms such as a phenyl group, a 1-naphthyl group, and a 2-naphthyl group; an aralkyl group having 7 to 38 carbon atoms such as a benzyl group and a diphenylmethyl group; and a group given by combining any of these.

Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a fluorine atom, a chlorine atom, a bromine atom, an iodine atom, a cyano group, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained. The heteroatom is preferably an oxygen atom. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and 3-oxocyclohexyl group.

Synthesis of sulfonium salts containing the anion represented by general formula (3A′) is described in detail, for example, in JP 2007-145797 A, JP 2008-106045 A, JP 2009-7327 A, and JP 2009-258695 A. For example, sulfonium salts described in JP 2010-215608 A, JP 2012-41320 A, JP 2012-106986 A, and JP 2012-153644 A are also suitably used.

Specific examples of the anion represented by the formula (3A) include, but are not limited to, those shown below. In the following general formulae, Ac is an acetyl group:

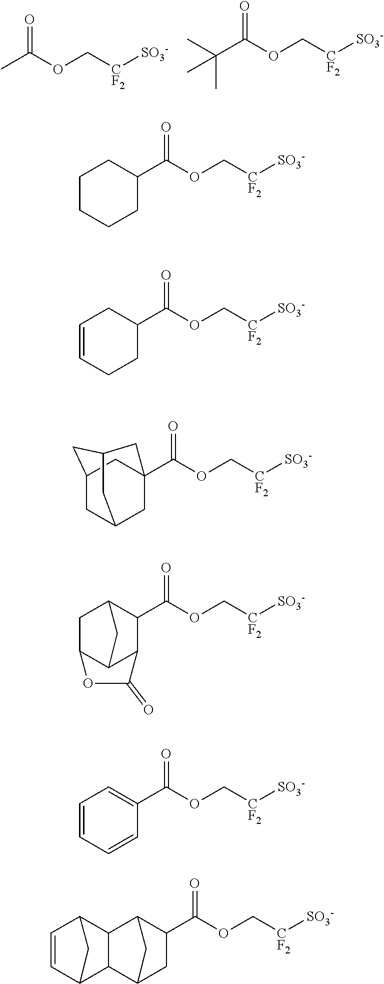

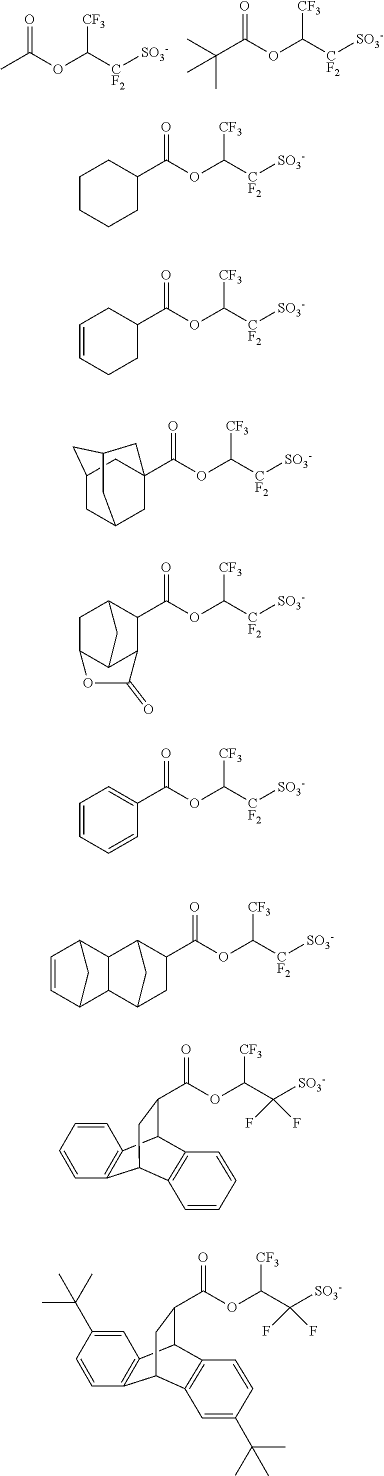

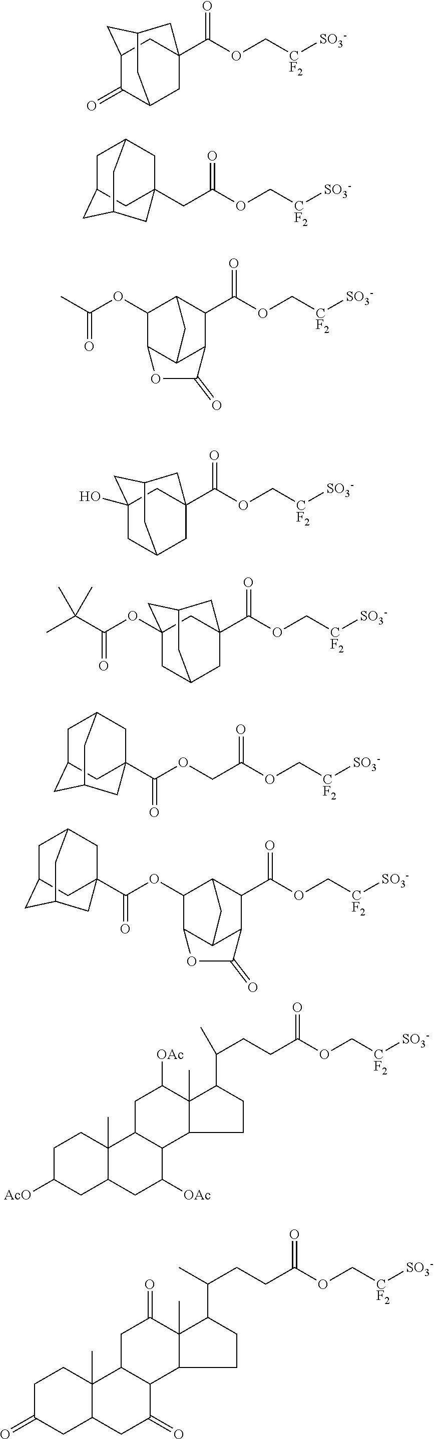

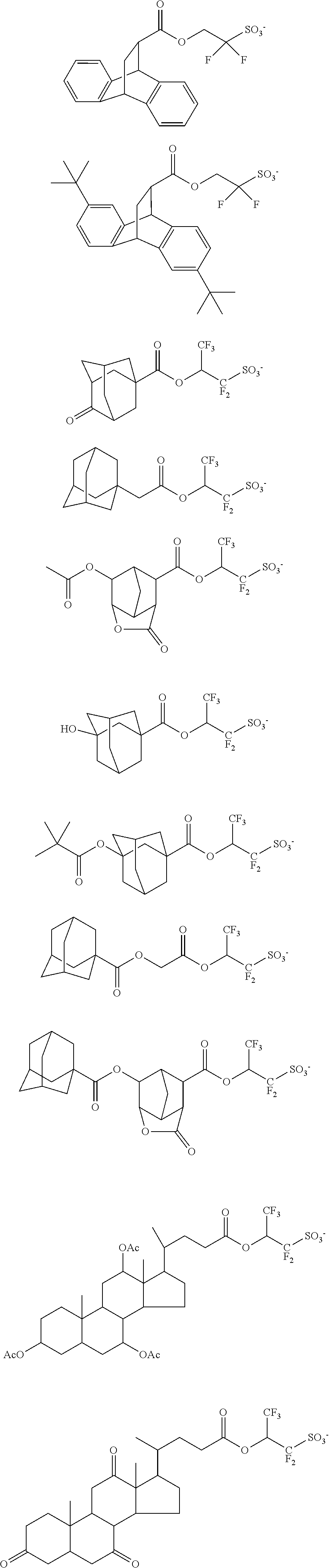

In the general formula (3B), R^{fb1} and R^{fb2} are each independently a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those shown as examples of the hydrocarbyl group represented by Ral in the general formula (3A′). R^{fb1} and R^{fb2} are preferably each a fluorine atom or a linear fluorinated alkyl group having 1 to 4 carbon atoms. R^{fb1} and R^{fb2} are optionally bound together to form a ring together with the group to which they are bound (—CF₂—SO₂—N—SO₂—CF₂—), and in this case the group resulting from bonding R^{fb1} and R^{fb2} together is preferably a fluorinated ethylene group or a fluorinated propylene group.

In the general formula (3C), R^{fc1}, R^{fc2}, and R^{fc3} are each independently a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those shown as examples of the hydrocarbyl group represented by R^{fa1} in the general formula (3A′). R^{fc1}, R^{fc2}, and R^{fc3} are preferably each a fluorine atom or a linear fluorinated alkyl group having 1 to 4 carbon atoms. R^{fc1} and R^{fc2} are optionally bound together to form a ring together with the group to which they are bound (—CF₂—SO₂—C—SO₂—CF₂—), and in this case the group resulting from bonding R^{fc1} and R^{fc2} together is preferably a fluorinated ethylene group or a fluorinated propylene group.

In the general formula (3D), R^{fd} is a hydrocarbyl group having 1 to 40 carbon atoms and optionally containing a heteroatom. The hydrocarbyl group may be saturated or unsaturated, and may be linear, branched, or cyclic. Specific examples thereof include those shown as examples of the hydrocarbyl group represented by R^{fa1} in the general formula (3A′).

Synthesis of sulfonium salts containing the anion represented by the general formula (3D) is described in detail in JP 2010-215608 A and JP 2014-133723 A.

Specific examples of the anion represented by the general formula (3D) include, but are not limited to, those shown in the following:

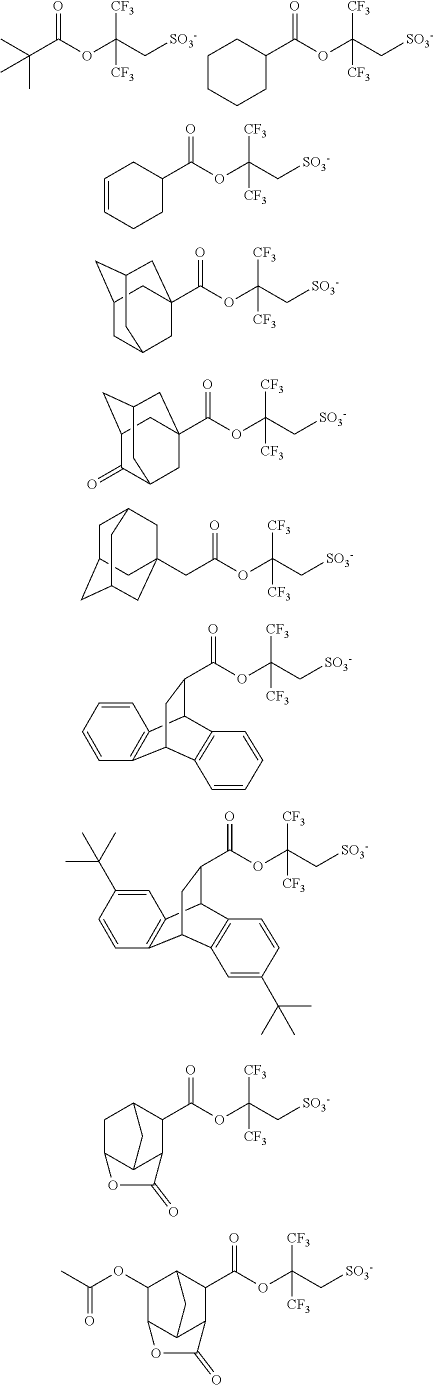

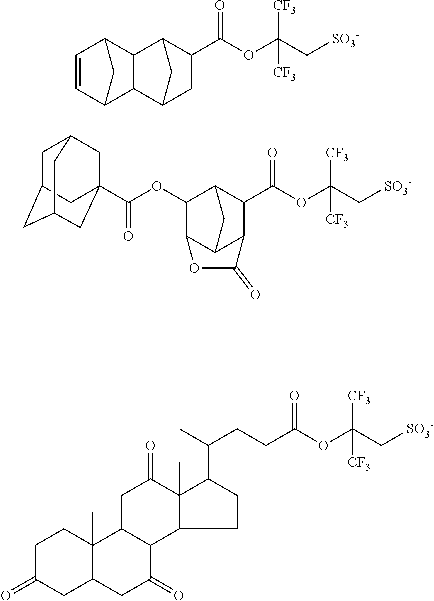

The photo-acid generator containing the anion represented by the general formula (3D) has no fluorine atom at the a position of the sulfo group, but has acidity enough to cleave acid-unstable groups in the base polymer because it has the two trifluoromethyl groups at the B position. Accordingly, use as a photo-acid generator is acceptable.

For the photo-acid generator, one represented by the following general formula (4) can also be suitably used.

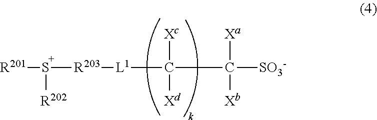

In the general formula (4), R²⁰¹ and R²⁰² are each independently a hydrocarbyl group having 1 to 30 carbon atoms and optionally containing a heteroatom. R²⁰³ is a hydrocarbylene group having 1 to 30 carbon atoms and optionally containing a heteroatom. Any two of R²⁰¹, R²⁰², and R²⁰³ are optionally bound together to form a ring together with the sulfur atom to which the two are bound. Specific examples of the ring in this case include those shown as examples of the ring that can be formed through bonding of any two of the three substituents bound to S⁺ together with the sulfur atom to which the two substituents are bound in the description of the general formula (a).

The hydrocarbyl group represented by R²⁰¹ and R²⁰², having 1 to 30 carbon atoms, may be saturated or unsaturated, and may be linear, branched, or cyclic. Specific examples thereof include an alkyl group having 1 to 30 carbon atoms such as a methyl group, an ethyl group, a n-propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, and a n-decyl group; a cyclic saturated hydrocarbyl group having 3 to 30 carbon atoms such as a cyclopentyl group, a cyclohexyl group, a cyclopentylmethyl group, a cyclopentylethyl group, cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group; an aryl group having 6 to 30 carbon atoms such as a phenyl group, a methylphenyl group, an ethylphenyl group, a n-propylphenyl group, an isopropylphenyl group, a n-butylphenyl group, an isobutylphenyl group, a sec-butylphenyl group, a tert-butylphenyl group, a naphthyl group, a methylnaphthyl group, an ethylnaphthyl group, a n-propylnaphthyl group, an isopropylnaphthyl group, a n-butylnaphthyl group, an isobutylnaphthyl group, a sec-butylnaphthyl group, a tert-butylnaphthyl group, and an anthracenyl group; and a group given by combining any of these. Some or all of the hydrogen atoms of the hydrocarbyl group may be each replaced with a group containing a heteroatom such as an oxygen atom, a sulfur atom, a nitrogen atom, and a halogen atom, some of the —CH₂— moieties of the hydrocarbyl group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic anhydride (—C(═O)—O—C(═O)—), or a haloalkyl group can be contained.

The hydrocarbylene group represented by R²⁰³, having 1 to 30 carbon atoms, may be saturated or unsaturated, and may be linear, branched, or cyclic. Specific examples thereof include an alkanediyl group having 1 to 30 carbon atoms such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; a cyclic saturated hydrocarbylene group having 3 to 30 carbon atoms such as a cyclopentanediyl group, a cyclohexanediyl group, a norbornanediyl group, and an adamantanediyl group; an arylene group having 6 to 30 carbon atoms such as a phenylene group, a methylphenylene group, an ethylphenylene group, a n-propylphenylene group, an isopropylphenylene group, a n-butylphenylene group, an isobutylphenylene group, a sec-butylphenylene group, a tert-butylphenylene group, a naphthylene group, a methylnaphthylene group, an ethylnaphthylene group, a n-propylnaphthylene group, an isopropylnaphthylene group, a n-butylnaphthylene group, an isobutylnaphthylene group, a sec-butylnaphthylene group, and a tert-butylnaphthylene group; and a group given by combining any of these. Some or all of the hydrogen atoms of the hydrocarbylene group may be each replaced with a group containing a heteroatom such as an oxygen atom, a sulfur atom, a nitrogen atom, and a halogen atom, some of the —CH₂— moieties of the hydrocarbylene group may be each replaced with a group containing a heteroatom such as an oxygen atom, a sulfur atom, and a nitrogen atom, and as a result, for example, a hydroxy group, a cyano group, a fluorine atom, a chlorine atom, a bromine atom, an iodine atom, a carbonyl group, an ether bond, an ester bond, a sulfonate bond, a carbonate bond, a lactone ring, a sultone ring, a carboxylic anhydride (—C(═O)—O—C(═O)—), or a haloalkyl group may be contained. The heteroatom is preferably an oxygen atom.

In the general formula (4), L¹ is a single bond, an ether bond, or a hydrocarbylene group having 1 to 20 carbon atoms and optionally containing a heteroatom. The hydrocarbylene group may be saturated or unsaturated, and may be linear, branched, or cyclic. Specific examples thereof include those shown as examples of the hydrocarbylene group represented by R²⁰³.

In the general formula (4), X^{a}, X^{b}, X^{c}, and X^{d} are each independently a hydrogen atom, a fluorine atom, or a trifluoromethyl group. It should be noted that at least one of X^{a}, X^{b}, X^{c}, and X^{d} is a fluorine atom or a trifluoromethyl group.

In the general formula (4), k is 0, 1, 2, or 3.

The photo-acid generator represented by the general formula (4) is preferably one represented by the following formula (4′).

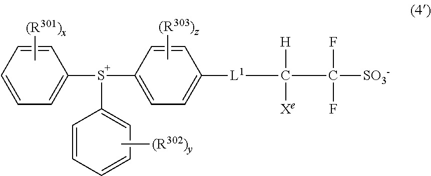

In the general formula (4′), L¹ is as specified above. X^{e} is a hydrogen atom or a trifluoromethyl group, and preferably a trifluoromethyl group. R³⁰¹, R³⁰², and R³⁰³ are each independently a hydrocarbyl group having 1 to 20 carbon atoms and optionally containing a heteroatom. The hydrocarbyl group may be saturated or unsaturated, and may be linear, branched, or cyclic. Specific examples thereof include those shown as examples of the hydrocarbyl group represented by R^{fa1} in the general formula (3A′). x and y are each independently 0, 1, 2, 3, 4, or 5. z is 0, 1, 2, 3, or 4.

Specific examples of the photo-acid generator represented by the general formula (4) include those shown as examples of a photo-acid generator represented by formula (2) in JP 2017-26980 A.

Among the photo-acid generators, a photo-acid generator containing the anion represented by the general formula (3A′) or (3D) causes small acid diffusion and exhibits superior solubility in solvents as well, thus being particularly preferred. The photo-acid generator represented by the general formula (4′) causes very small acid diffusion, thus being particularly preferred.

Applicable as other acid generators are a sulfonium salt and an iodonium salt represented by the following general formulae (5-1) and (5-2), respectively, each containing an anion having an aromatic ring or aromatic rings substituted with an iodine atom or iodine atoms:

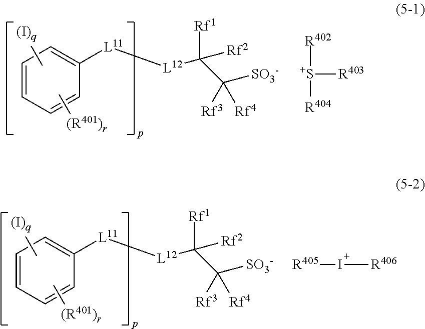

In the general formulae (5-1) and (5-2), p is 1, 2, or 3. q is 1, 2, 3, 4, or 5. r is 0, 1, 2, or 3. It should be noted that 1≤q+r≤5 is satisfied. q is preferably 1, 2, or 3, and more preferably 2 or 3. r is preferably 0, 1, or 2.

In the general formulae (5-1) and (5-2), L¹¹ is a single bond, an ether bond or an ester bond, or a saturated hydrocarbylene group having 1 to 6 carbon atoms and optionally containing an ether bond or an ester bond. The saturated hydrocarbylene group may be linear, branched, or cyclic.

In the general formulae (5-1) and (5-2), L¹² is a single bond or a divalent linking group having 1 to 20 carbon atoms if p is 1, and a (p+1)-valent linking group having 1 to 20 carbon atoms if p is 2 or 3, and the linking group may contain an oxygen atom, a sulfur atom, or a nitrogen atom.

In the general formulae (5-1) and (5-2), R⁴⁰¹ is: a hydroxy group, a carboxy group, a fluorine atom, a chlorine atom, a bromine atom, or an amino group; a hydrocarbyl group having 1 to 20 carbon atoms, hydrocarbyloxy group having 1 to 20 carbon atoms, hydrocarbylcarbonyl group having 2 to 20 carbon atoms, hydrocarbyloxycarbonyl group having 2 to 20 carbon atoms, hydrocarbylcarbonyloxy group having 2 to 20 carbon atoms, or hydrocarbylsulfonyloxy group having 1 to 20 carbon atoms, each optionally containing a fluorine atom, a chlorine atom, a bromine atom, a hydroxy group, an amino group, or an ether bond; or —N(R^{401A}) (R^{401B}) N(R^{401C})—C(═O)—R^{401D}, or —N(R^{401C})—C(═O)—O—R^{401D}. R^{401A} and R^{401B} are each independently a hydrogen atom or a saturated hydrocarbyl group having 1 to 6 carbon atoms. R^{401C} is a hydrogen atom or a saturated hydrocarbyl group having 1 to 6 carbon atoms, and may contain a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{401D} is an aliphatic hydrocarbyl group having 1 to 16 carbon atoms, an aryl group having 6 to 14 carbon atoms, or an aralkyl group having 7 to 15 carbon atoms, and may contain a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be saturated or unsaturated, and may be linear, branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be each linear, branched, or cyclic. If p and/or r are/is 2 or more, R⁴⁰¹ groups are identical to or different from each other.

Among them, for example, R⁴⁰¹ is preferably a hydroxy group,—N(R^{401C})—C(═O)—R^{401D}, —N(R^{401C})—C(═O)—O—R^{401D}, a fluorine atom, a chlorine atom, a bromine atom, a methyl group, or a methoxy group.

In the general formulae (5-1) and (5-2), Rfl to Rf⁴ are each independently a hydrogen atom, a fluorine atom, or a trifluoromethyl group, and at least one of them is a fluorine atom or a trifluoromethyl group. Rf¹ and Rf² may be combined to form a carbonyl group. In particular, Rf³ and Rf⁴ are preferably each a fluorine atom.

In the general formulae (5-1) and (5-2), R⁴⁰² to R⁴⁰⁶ are each independently a halogen atom or a hydrocarbyl group having 1 to 20 carbon atoms and optionally containing a heteroatom. The hydrocarbyl group may be saturated or unsaturated, and may be linear, branched, or cyclic. Specific examples thereof include those shown as examples of the hydrocarbyl group represented by R² in the description of the general formula (a). Some or all of the hydrogen atoms of the hydrocarbyl group may be each replaced with a hydroxy group, a carboxy group, a halogen atom, a cyano group, a nitro group, a mercapto group, a sultone ring, a sulfo group, or a sulfonium salt-containing group, and some of the —CH₂— moieties of the hydrocarbyl group may be each replaced with an ether bond, an ester bond, a carbonyl group, an amide bond, a carbonate bond, or a sulfonate bond. R⁴⁰² and R⁴⁰³ are optionally bound together to form a ring together with the sulfur atom to which they are bound. Specific examples of the ring in this case include those shown as examples of the ring that can be formed through bonding of any two of the three substituents bound to S⁺ together with the sulfur atom to which the two substituents are bound in the description of the general formula (a).

Specific examples of the cation of the sulfonium salt represented by the general formula (5-1) include those shown in paragraphs [0102] to [0125] of JP 2024-3744 A, those shown in paragraphs [0070] to [0085] of JP 2023-169812 A, and that represented by the general formula (d3). Specific examples of the cation of the iodonium salt represented by the formula (5-2) include those shown in paragraph [0181] of JP 2024-000259 A.

Specific examples of the anion of the onium salt represented by the general formula (5-1) or (5-2) include, but are not limited to, those shown in the following:

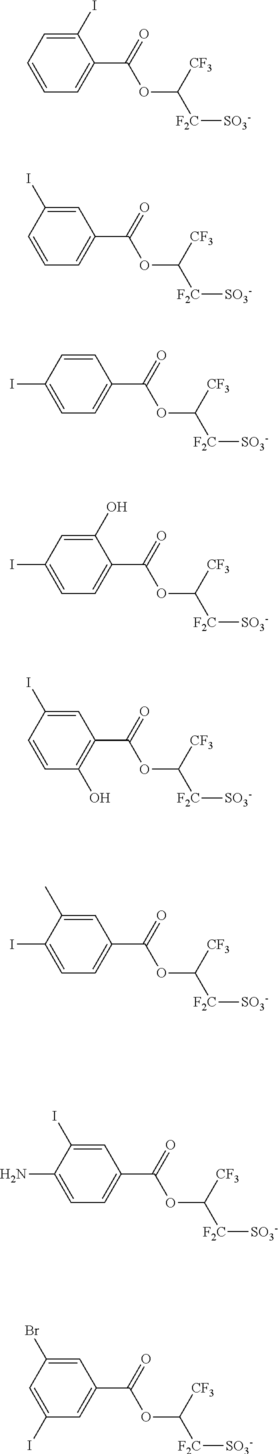

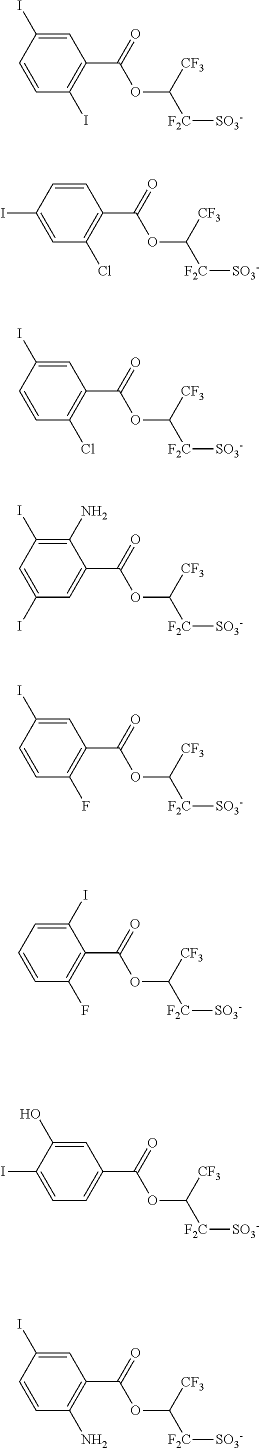

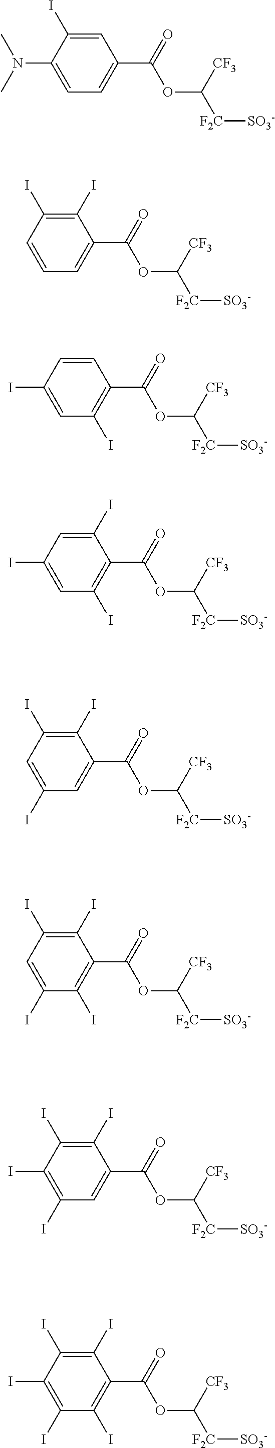

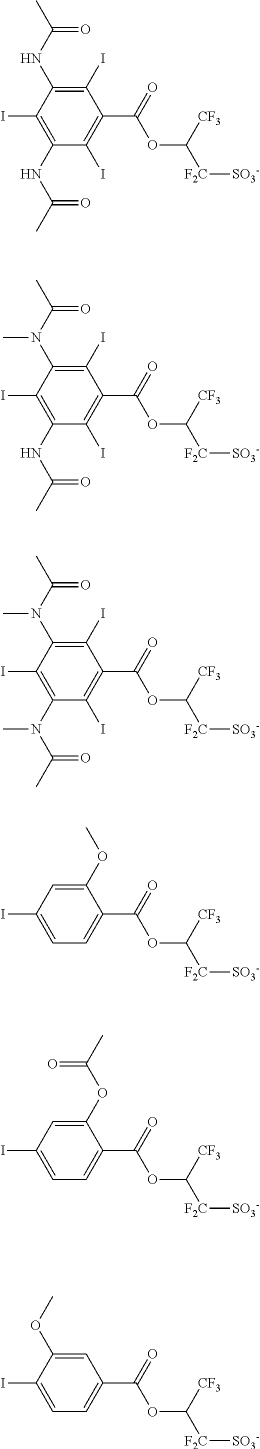

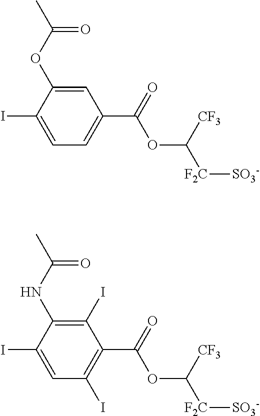

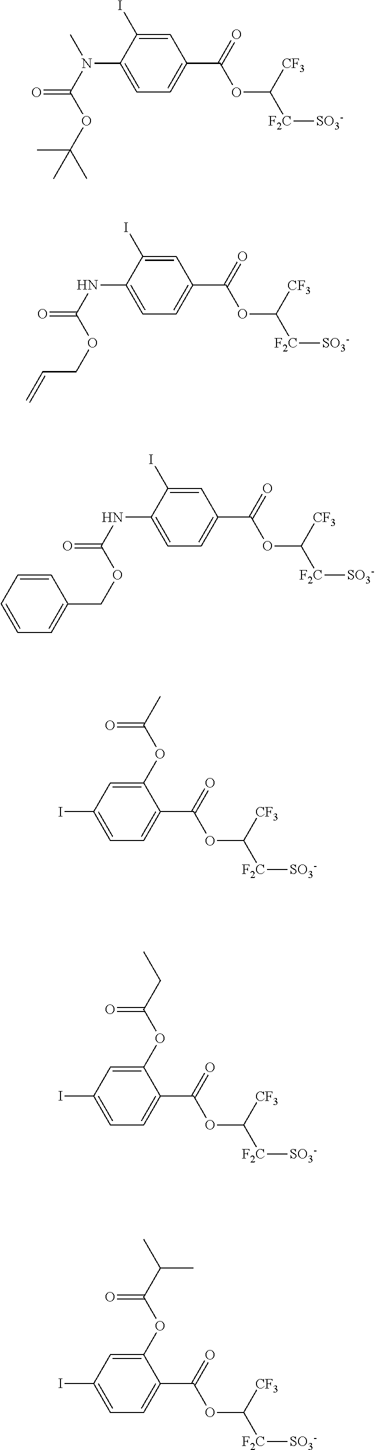

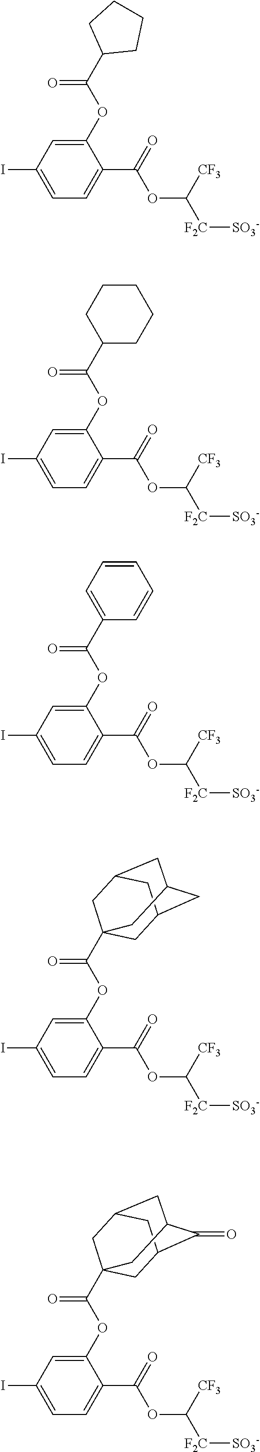

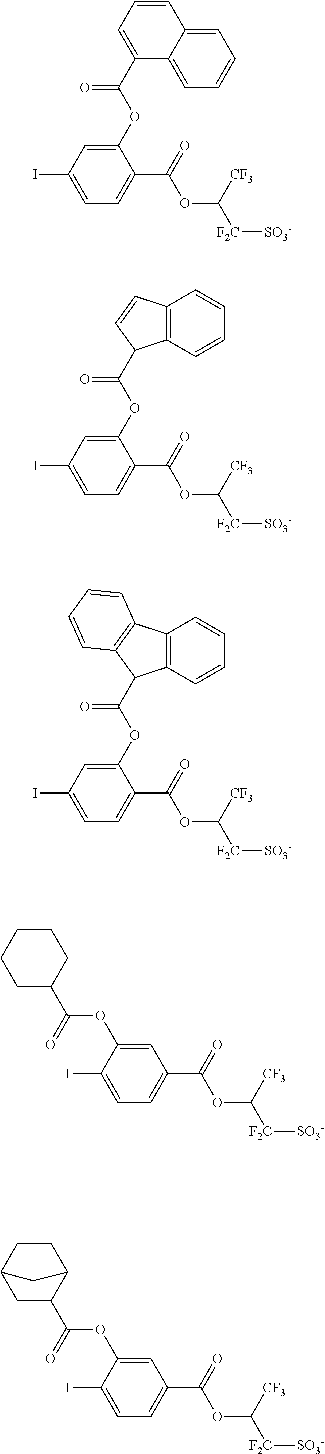

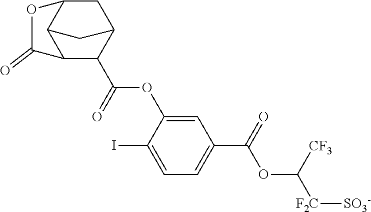

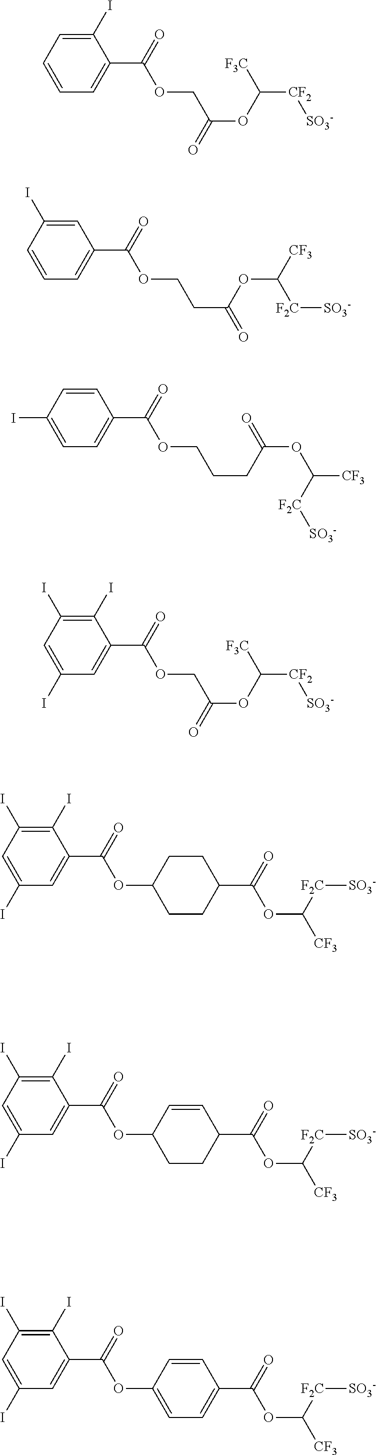

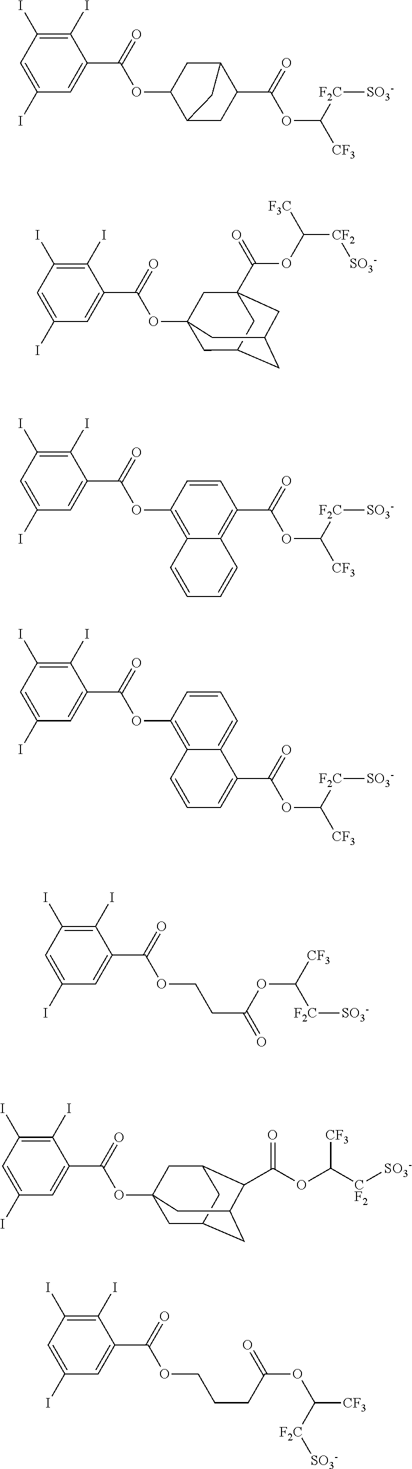

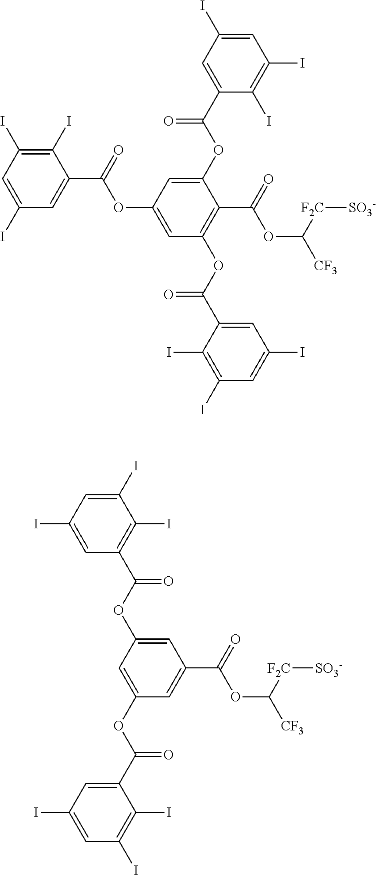

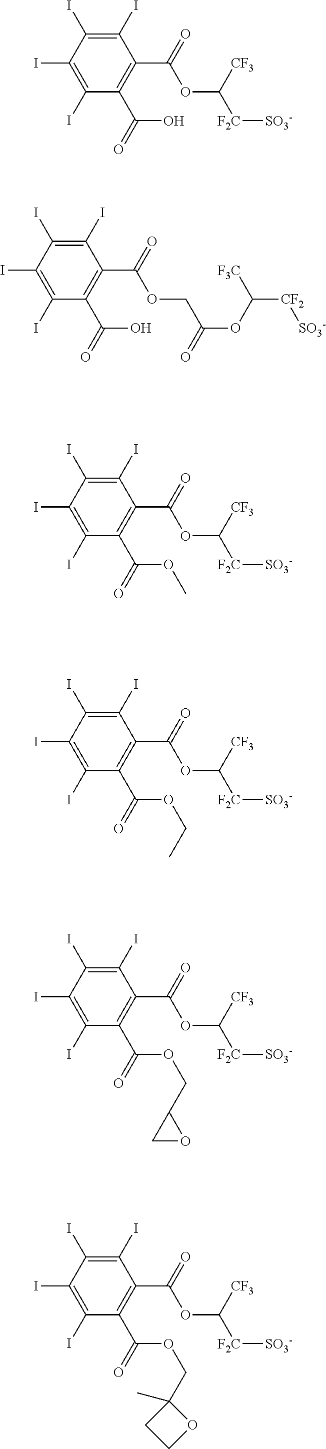

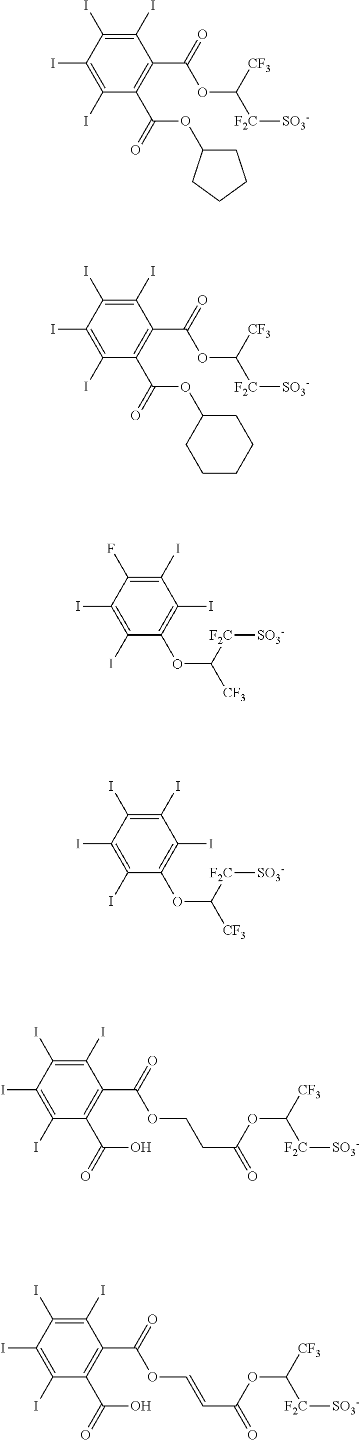

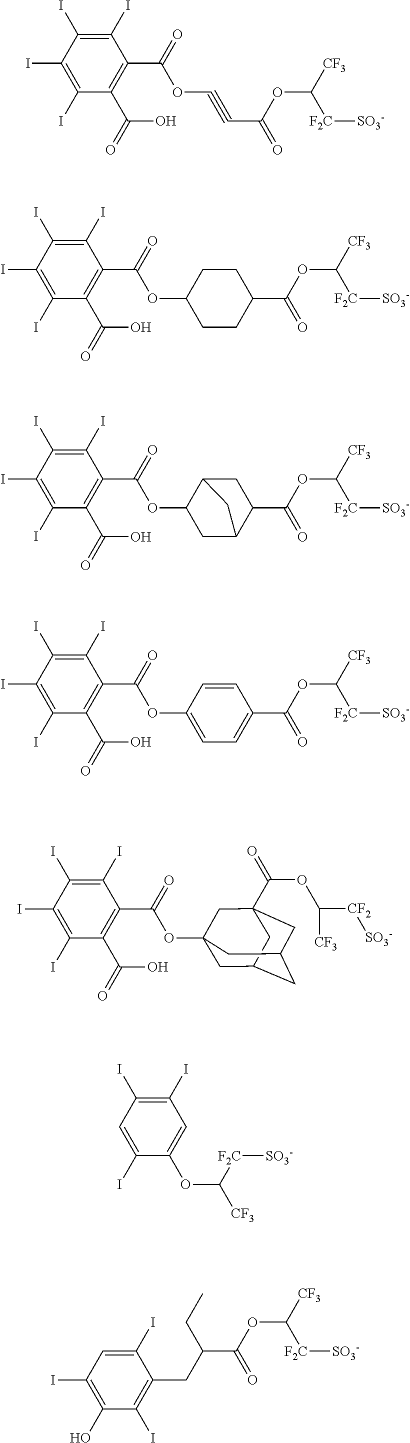

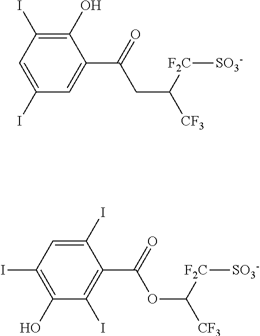

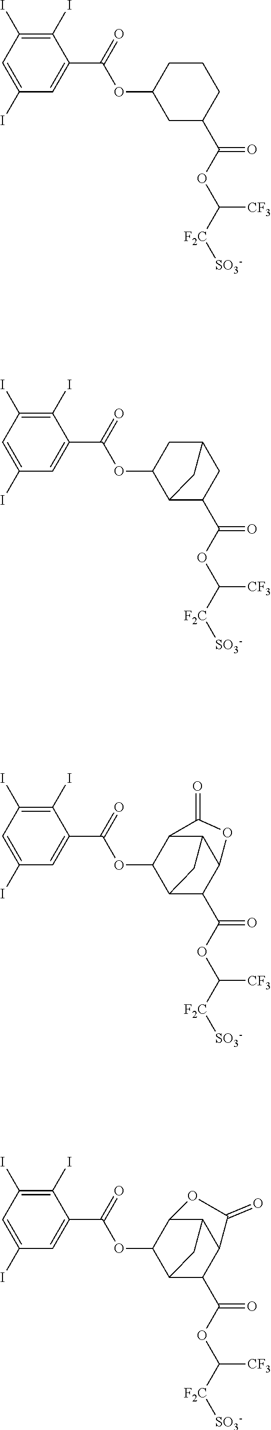

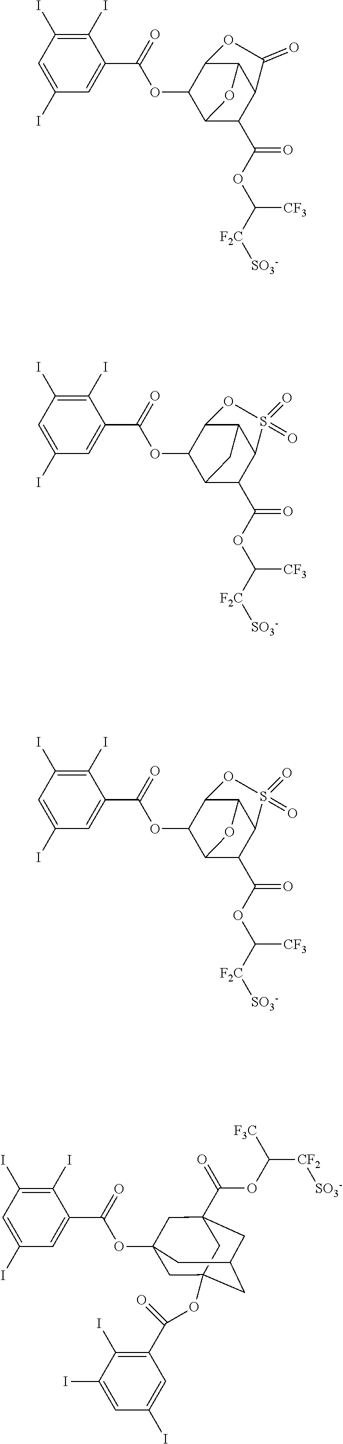

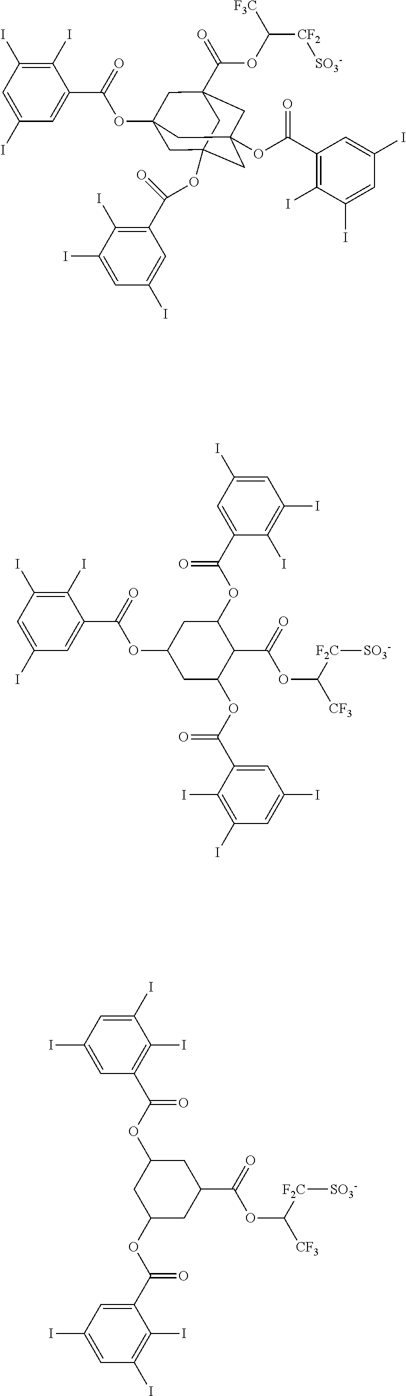

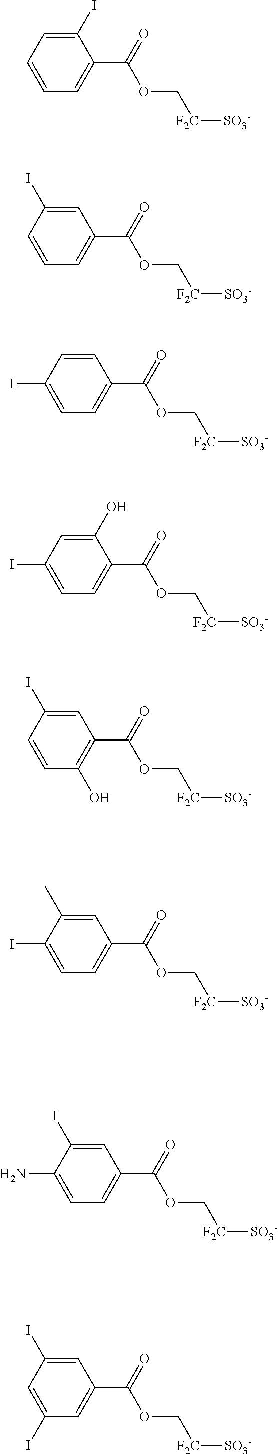

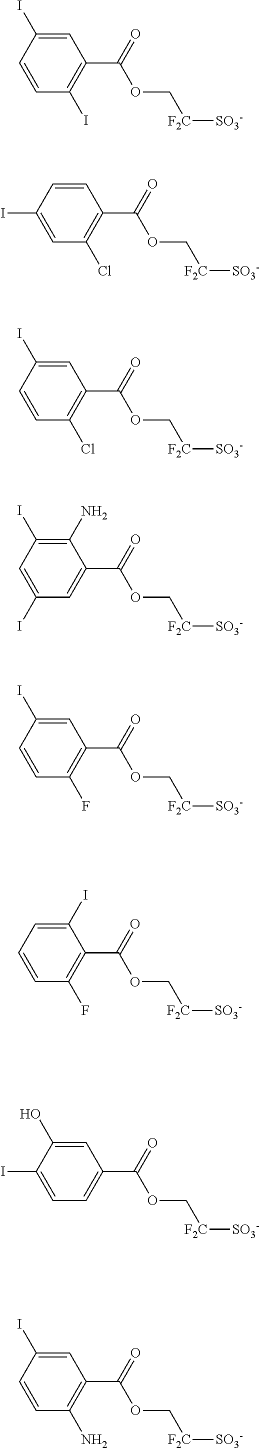

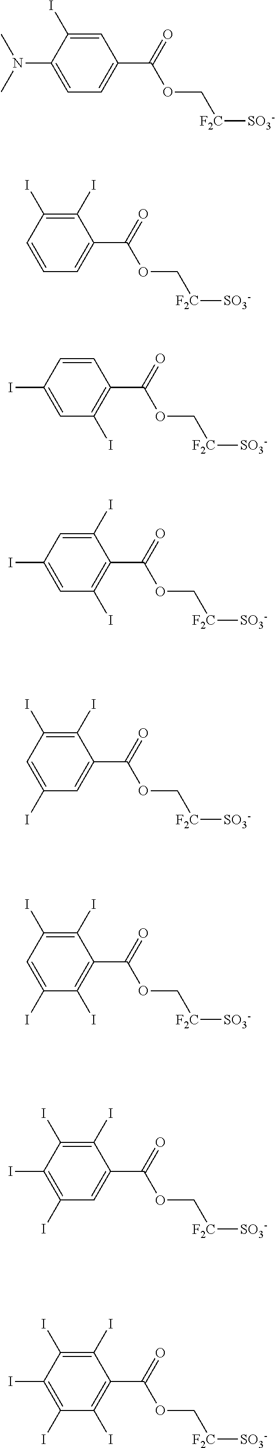

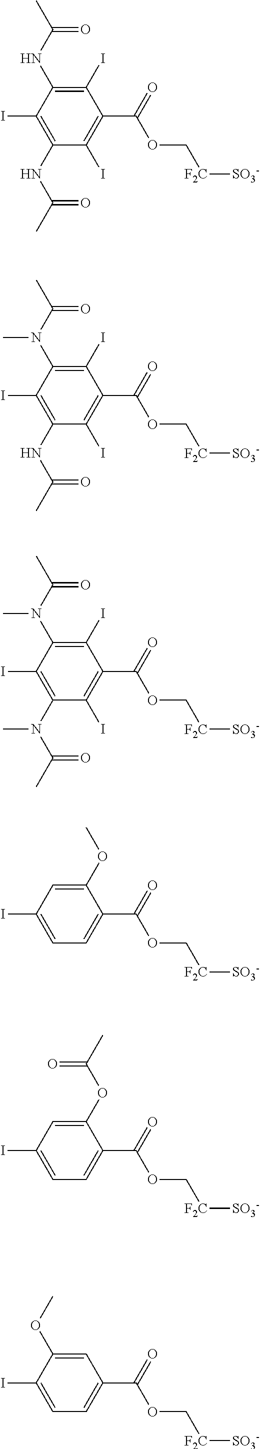

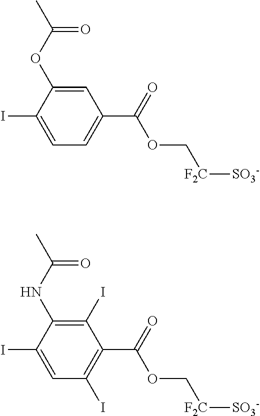

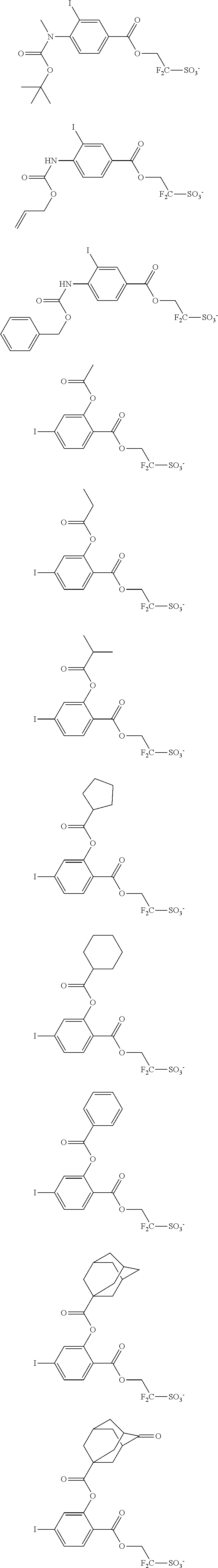

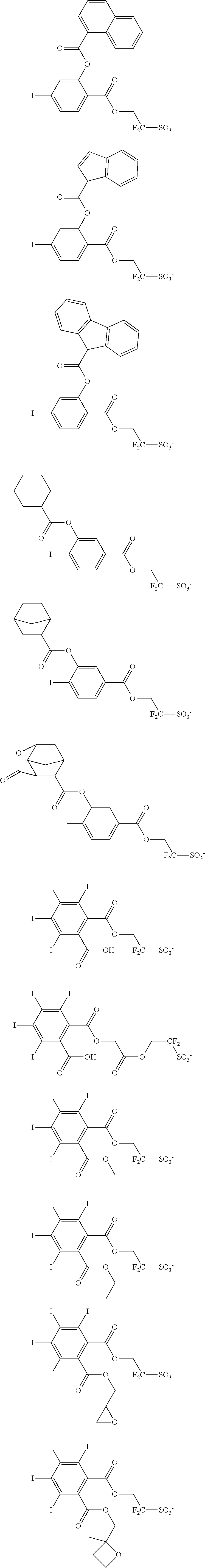

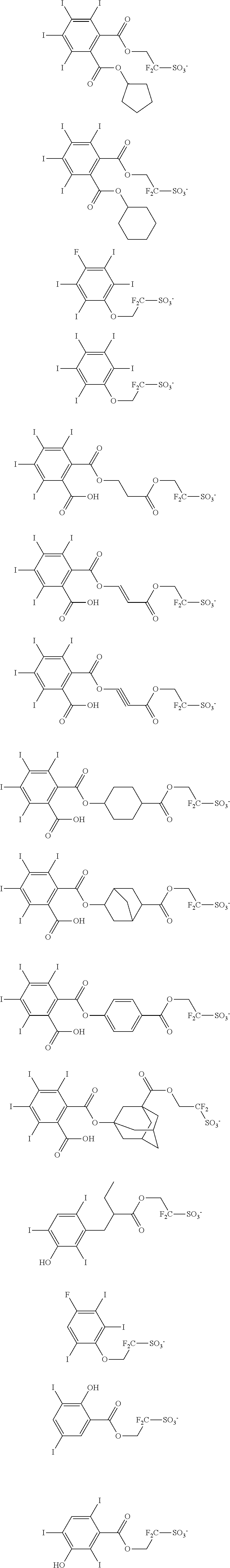

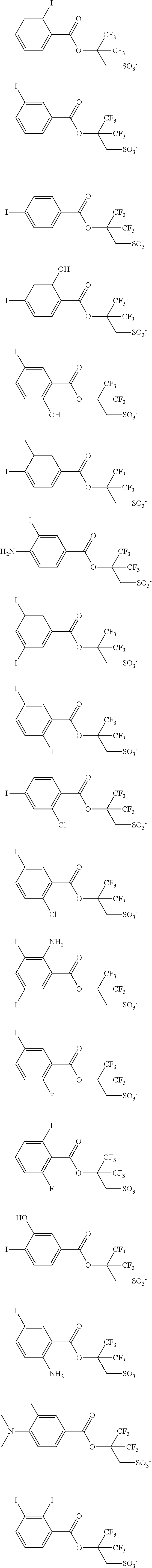

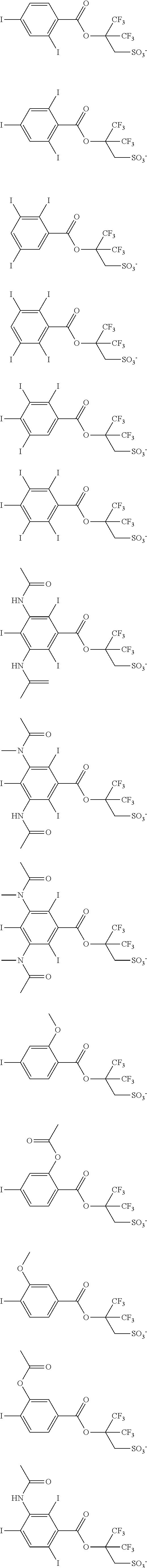

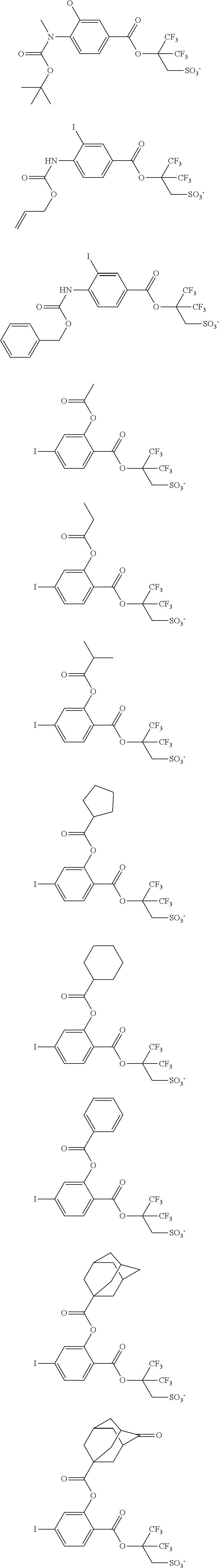

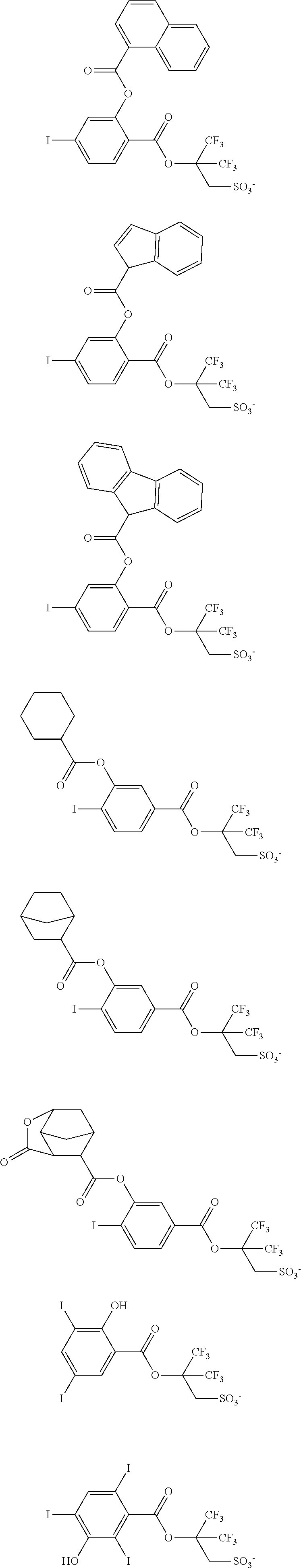

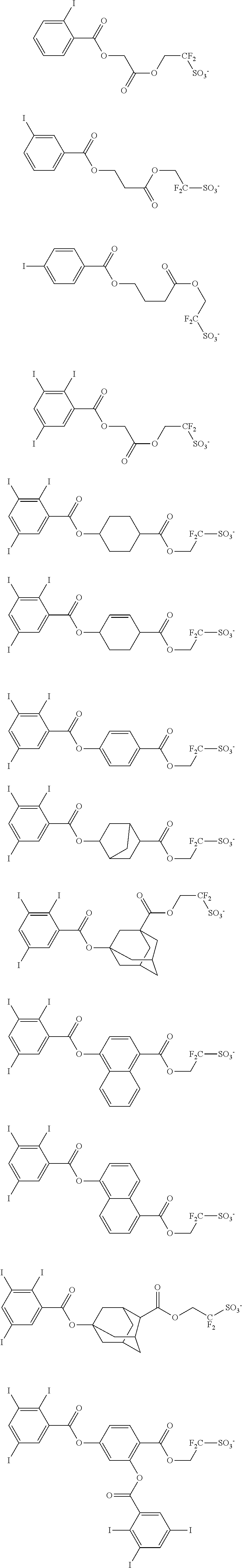

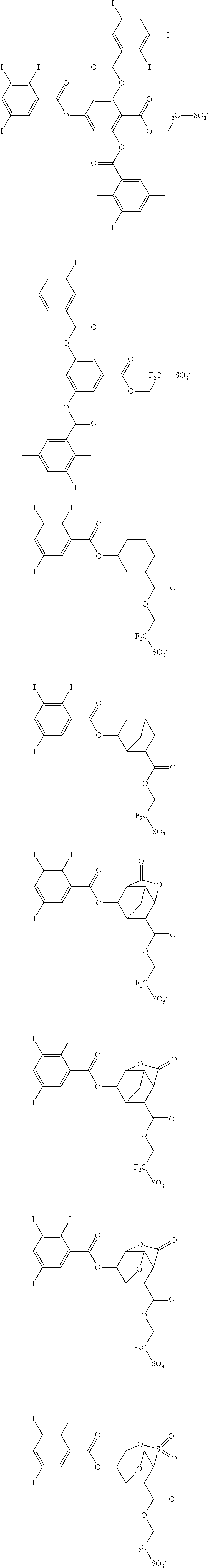

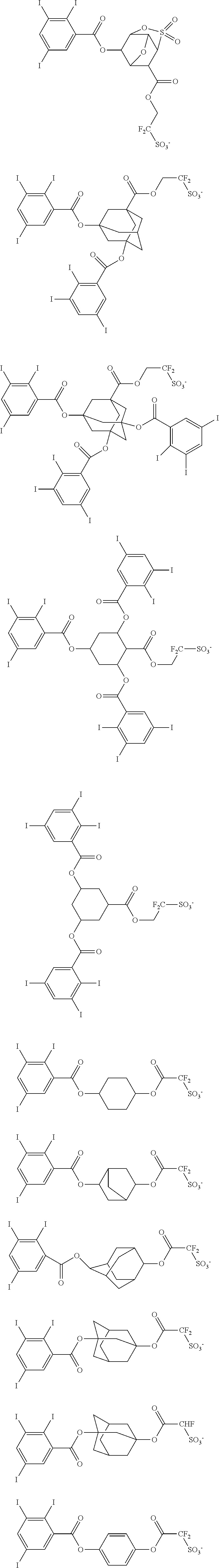

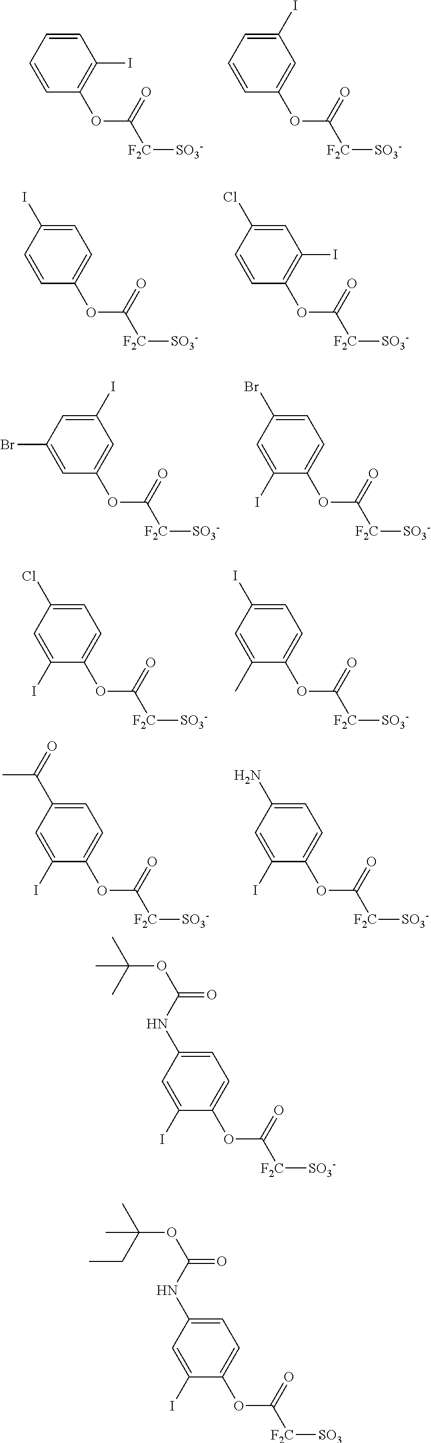

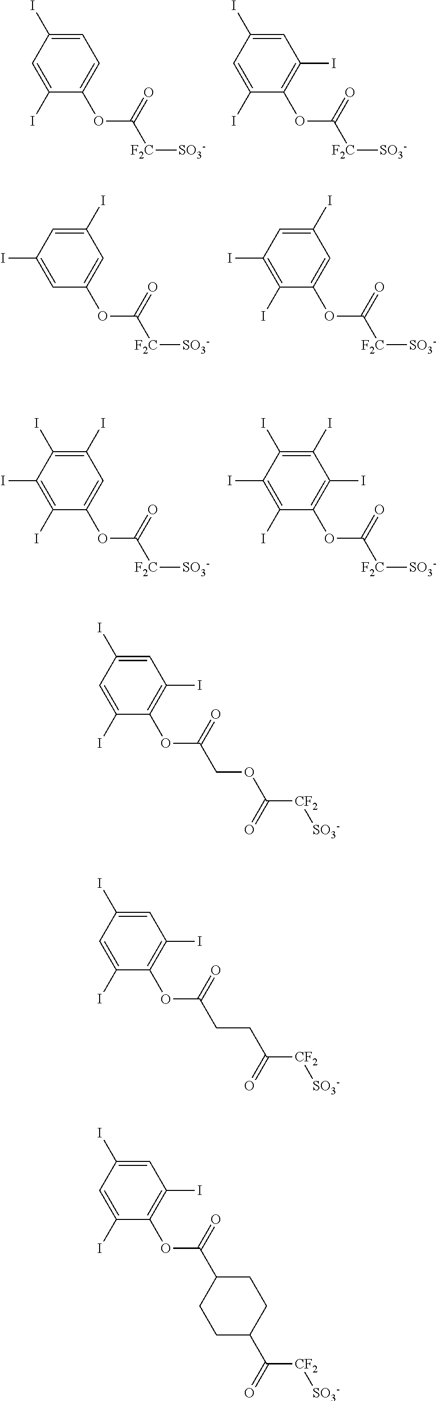

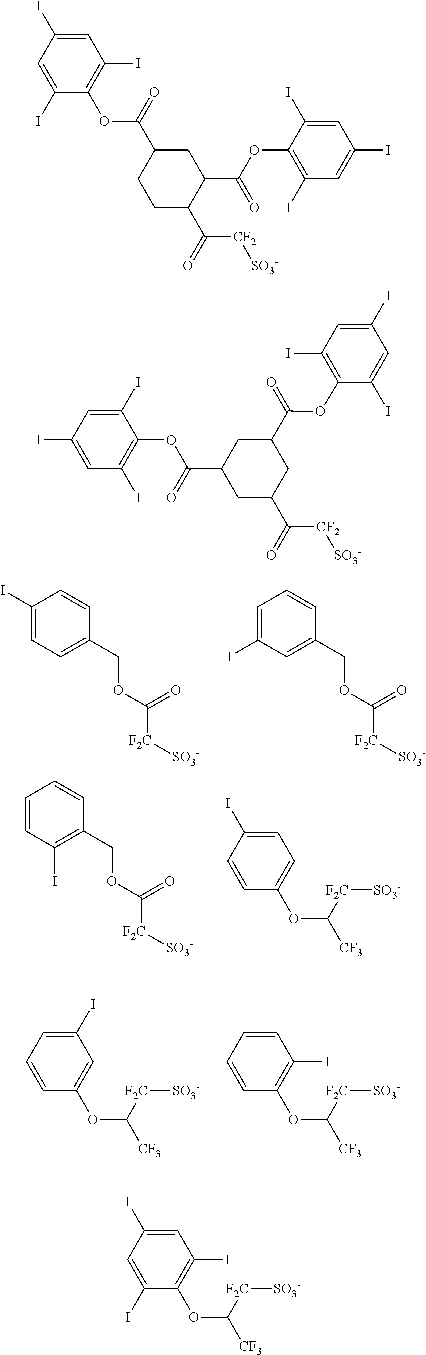

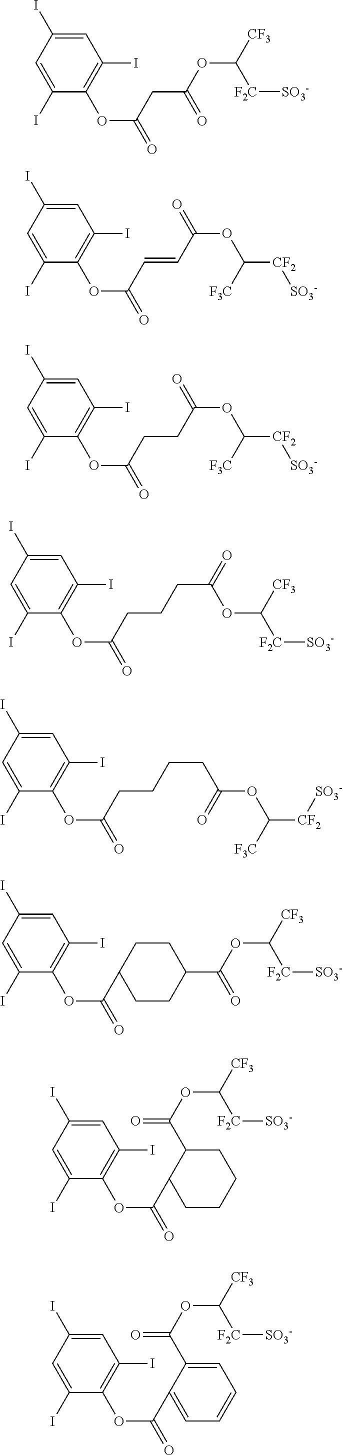

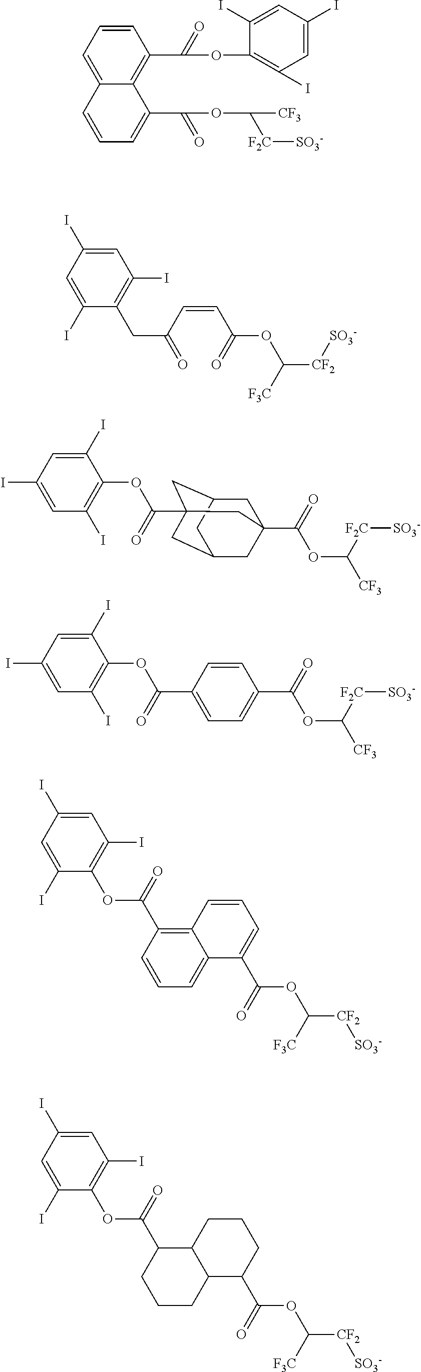

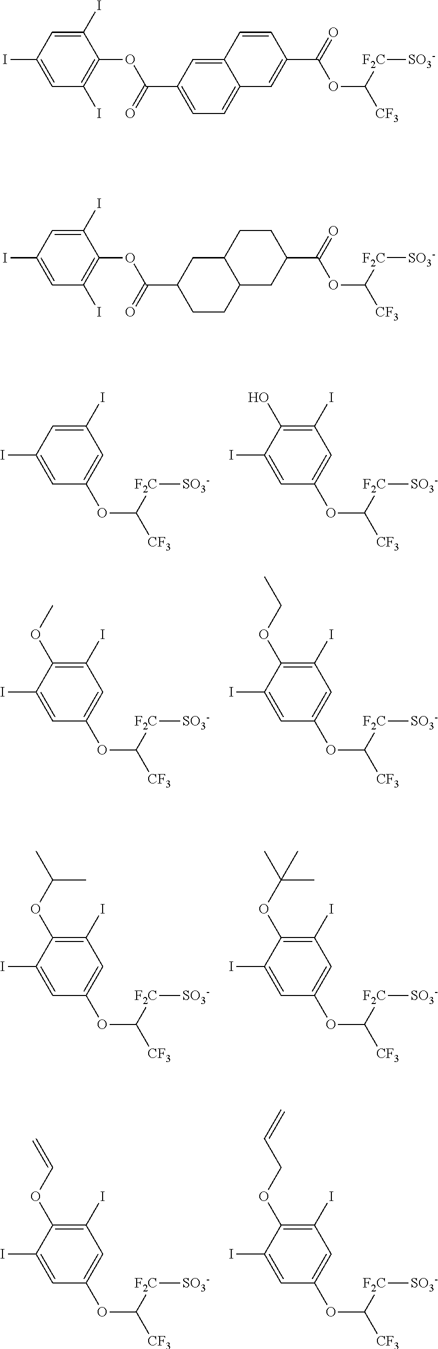

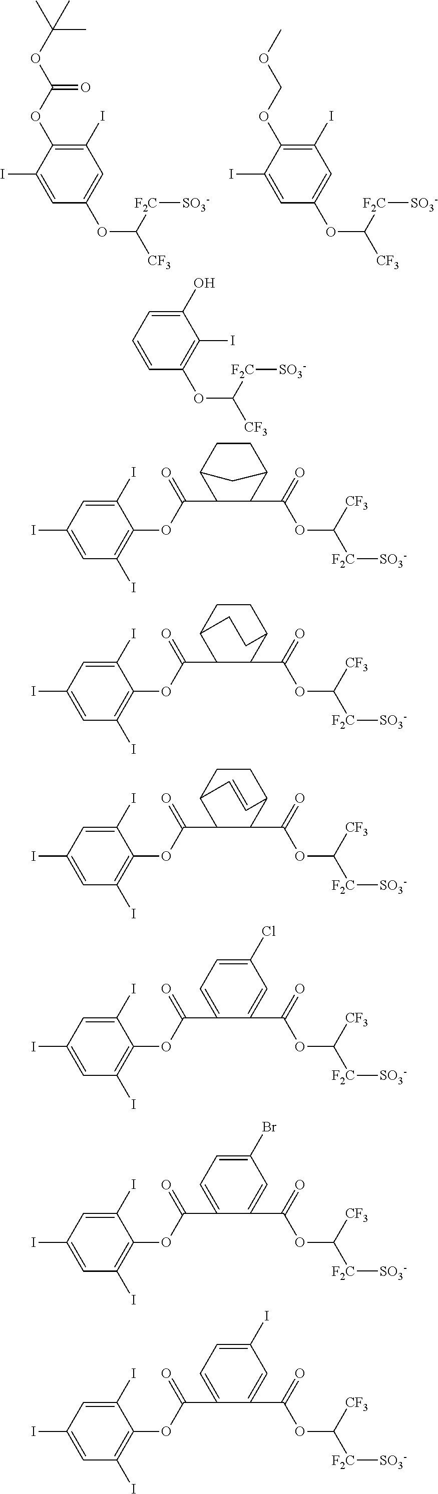

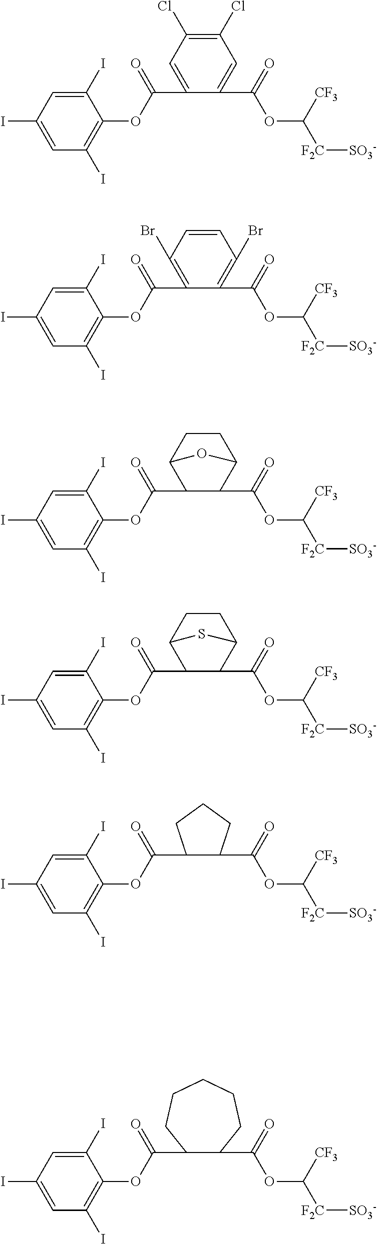

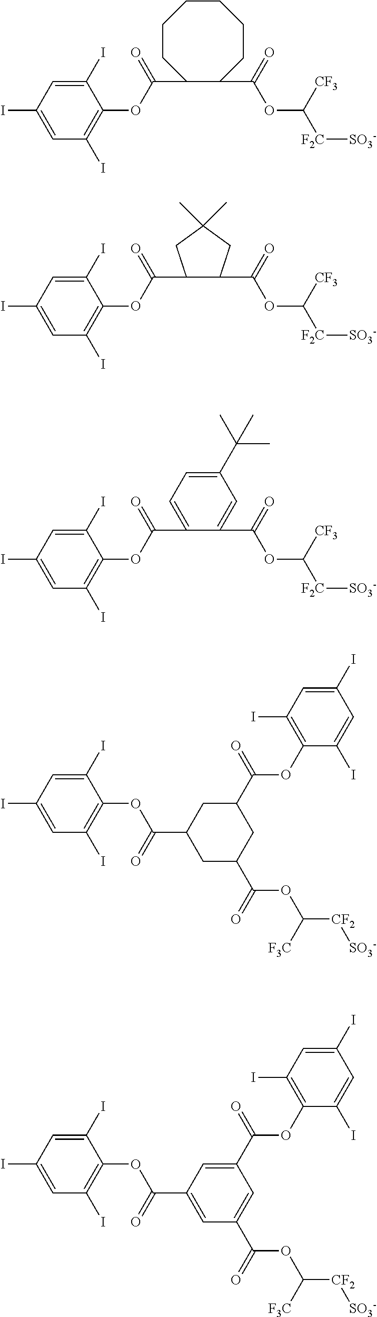

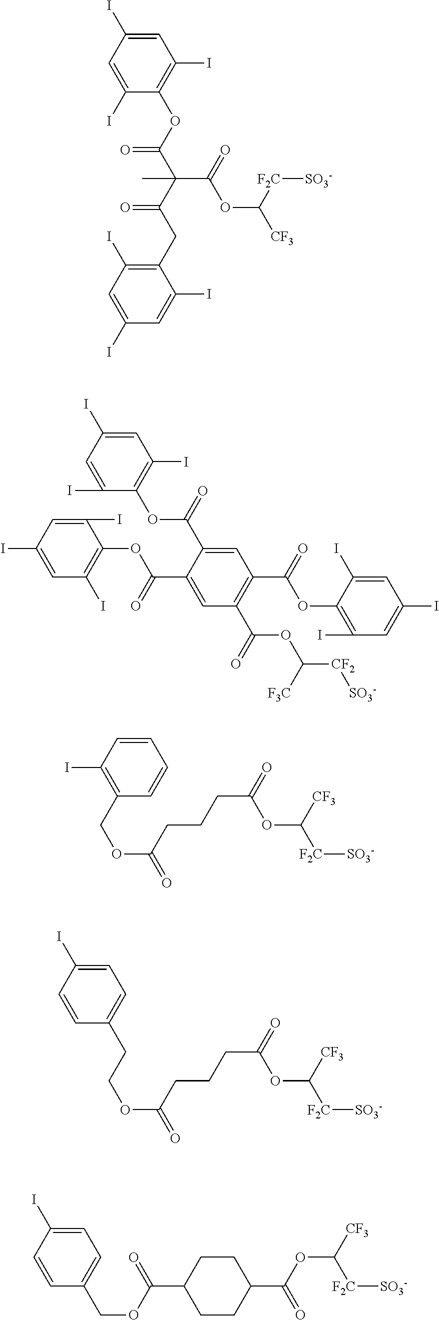

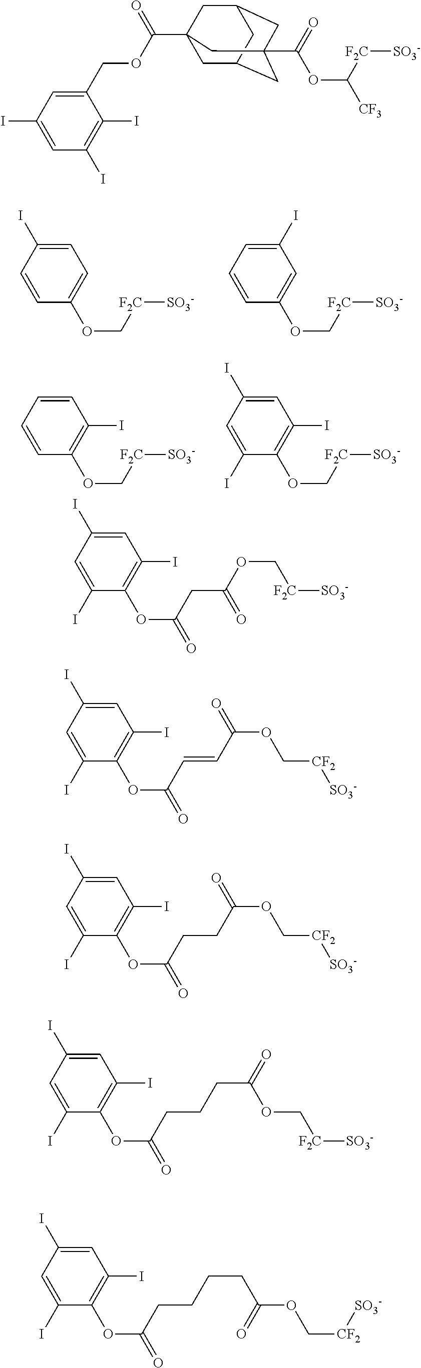

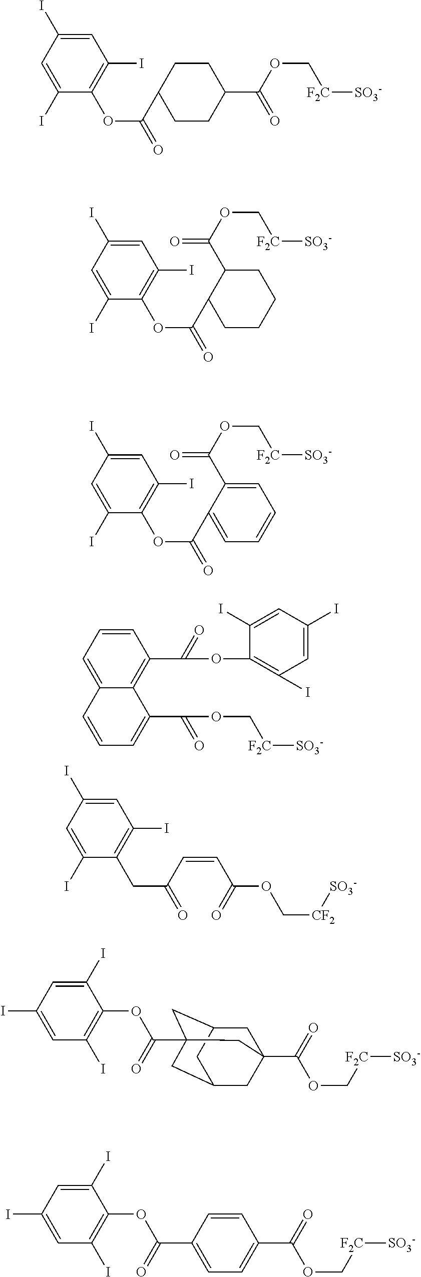

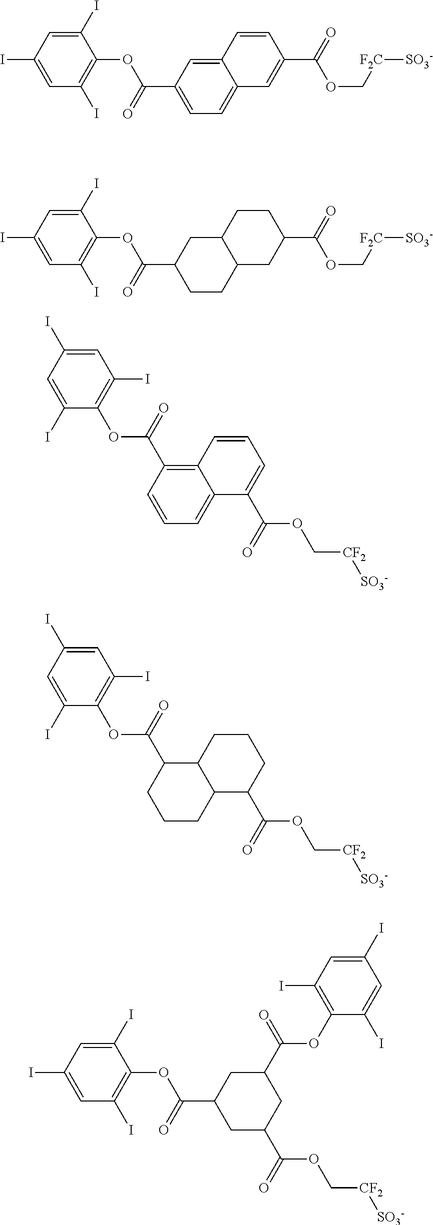

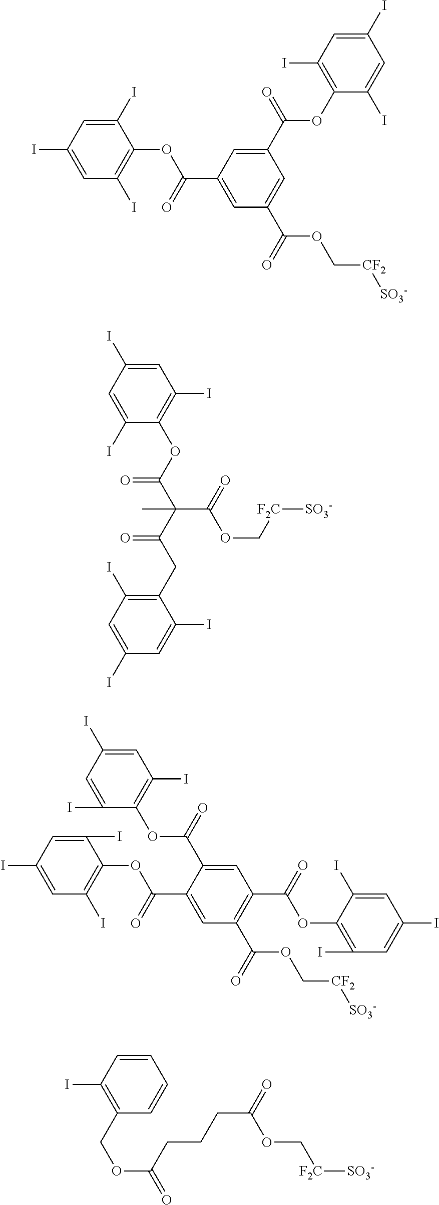

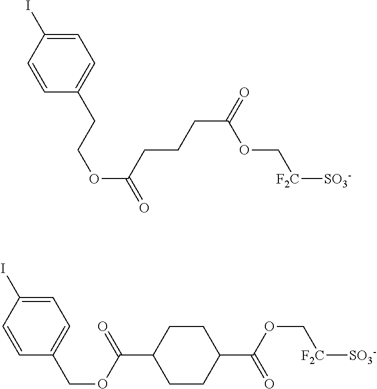

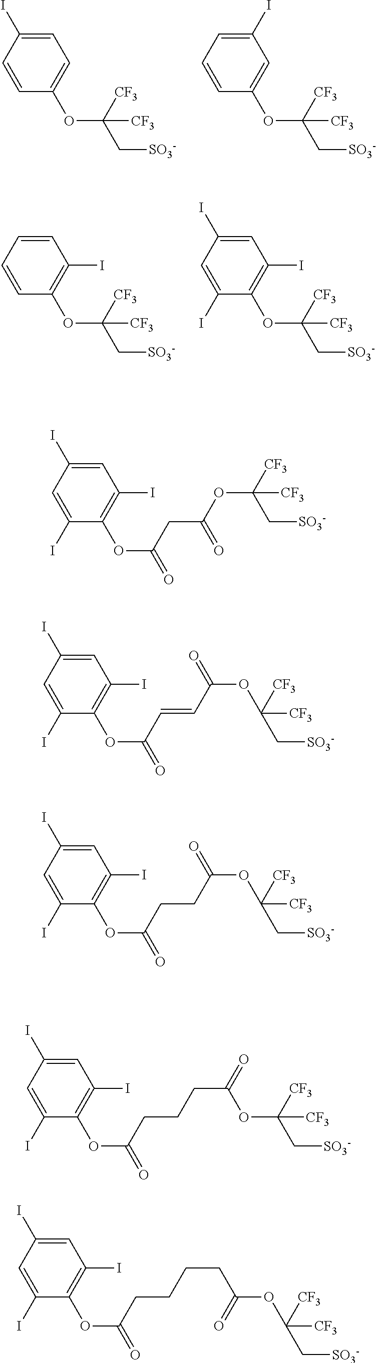

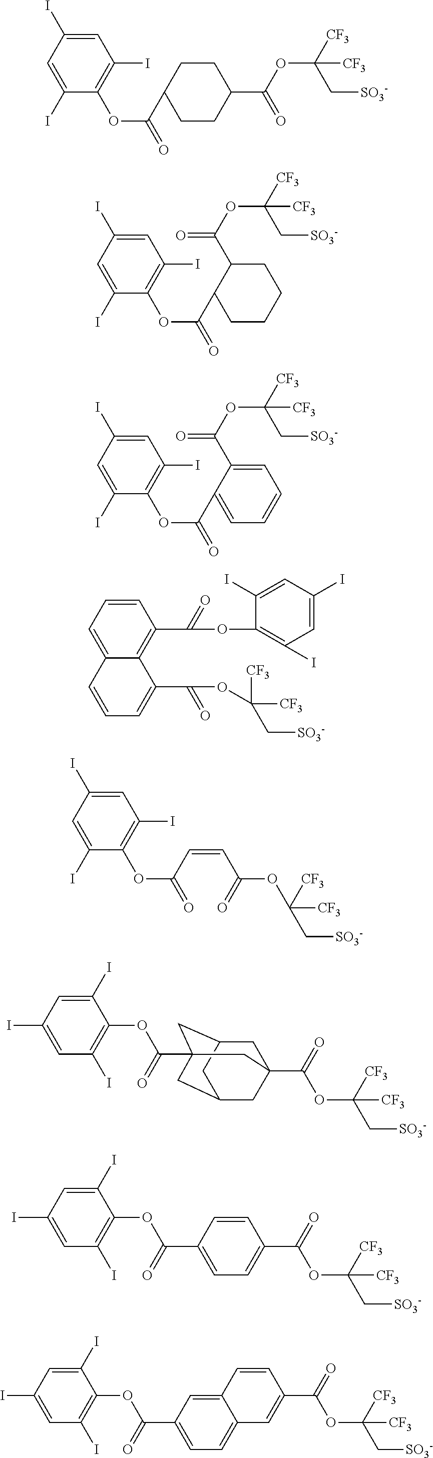

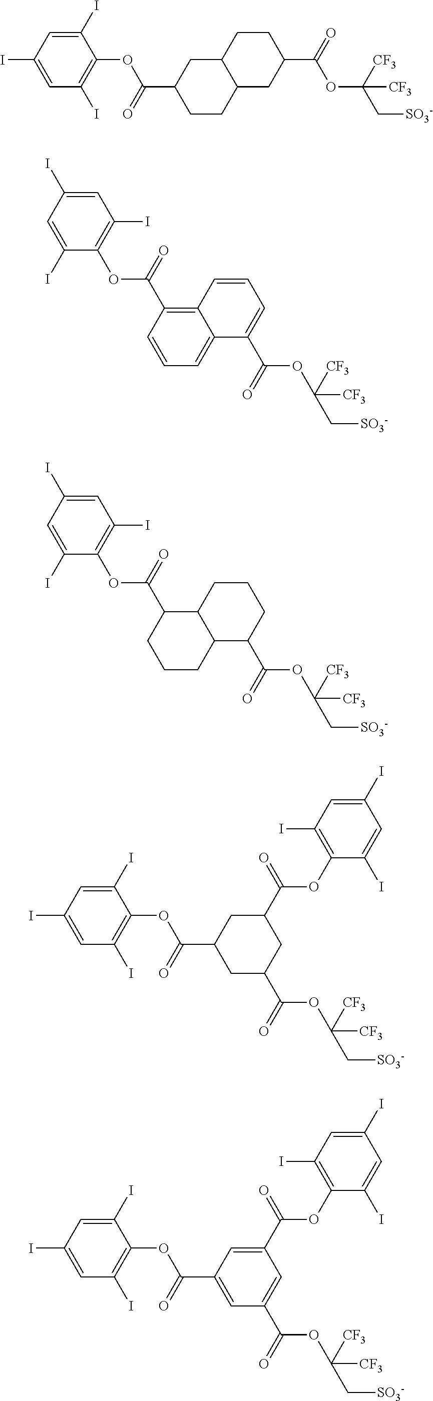

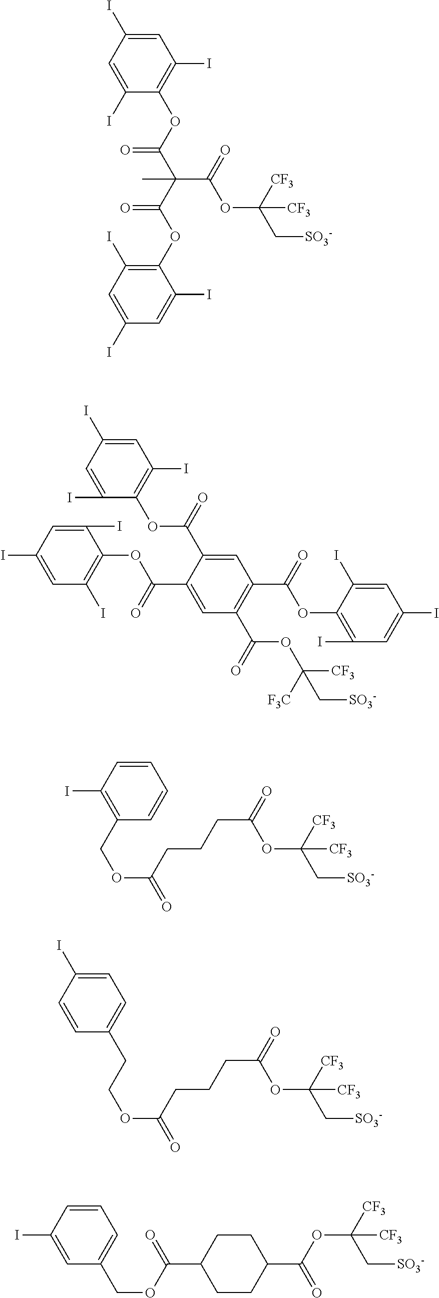

If the chemically amplified resist composition of the present invention contains acid generator (D), the amount of acid generator (D) contained therein is preferably 0.1 to 40 parts by mass, and more preferably 0.5 to 20 parts by mass per 80 parts by mass of base polymer (A). The case that the amount of the acid generator to be added as component (D) falls within that range is preferred because good resolution results and problems of undesired matters are reliably avoided after development of a resist film or during peeling-off of a resist film. For acid generator (D), one acid generator may be used alone; alternatively, two or more acid generators may be used in combination.

### [Surfactant (E)]

The chemically amplified resist composition of the present invention may further contain a surfactant as component (E). Surfactant (E) is preferably a surfactant that is insoluble or poorly soluble in water and soluble in alkaline developers, or a surfactant that is insoluble or poorly soluble in water and alkaline developers. For such surfactants, reference can be made to those described in JP 2010-215608 A and JP 2011-16746 A.

For example, among the surfactants described in the publications, FC-4430 (manufactured by 3M Company), Surflon (registered trademark) S-381 (manufactured by AGC Seimi Chemical Co., Ltd.), OLFINE (registered trademark) E1004 (manufactured by Nissin Chemical Industry Co., Ltd.), KH-20 and KH-30 (manufactured by AGC Seimi Chemical Co., Ltd.), and an oxetane ring-opening polymerization product represented by the following formula (surf-1) are preferred as the surfactant that is insoluble or poorly soluble in water and alkaline developers:

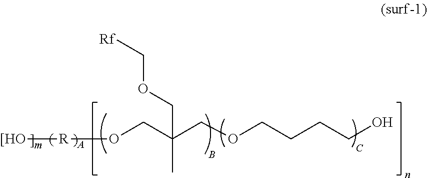

Here, R, Rf, A, B, C, m, and n are applied only to the general formula (surf-1), independently of the description shown above. R is a di- to tetravalent aliphatic group having 2 to 5 carbon atoms. Examples of the aliphatic group include an ethylene group, a 1,4-butylene group, a 1,2-propylene group, a 2,2-dimethyl-1,3-propylene group, and a 1,5-pentylene group as divalent aliphatic groups, and the following groups as tri- or tetravalent aliphatic groups:

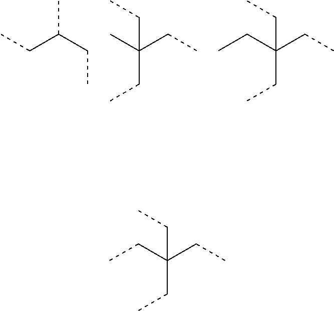

wherein each dashed line indicates bonding, and these are partial structures derived from glycerol, trimethylolethane, trimethylolpropane, and pentaerythritol, respectively.

Among them, for example, a 1,4-butylene group and a 2,2-dimethyl-1,3-propylene group are preferred.

Rf is a trifluoromethyl group or a pentafluoroethyl group, and preferably a trifluoromethyl group. m is an integer of 0 to 3, n is an integer of 1 to 4, and the sum of n and m is the valence of R, being an integer of 2 to 4. A is 1. B is an integer of 2 to 25, and preferably an integer of 4 to 20. C is an integer of 0 to 10, and preferably 0 or 1. The constituent units shown in the general formula (surf-1) are not intended to specify the order of them, and may be bound in a block-by-block manner or randomly. Production of surfactants based on partially fluorinated oxetane ring-opening polymerization products is described in detail in, for example, U.S. Pat. No. 5,650,483 A.

In the case that no resist protection film is used in ArF immersion lithography, the surfactant that is insoluble or poorly soluble in water and soluble in alkaline developers functions to reduce the permeation and leaching of water by orienting on the surface of a resist film. Accordingly, this surfactant is useful for inhibiting the elution of water-soluble components from resist films to reduce damage to aligners, and also useful in development with alkaline aqueous solution after exposure or after post-exposure bake (PEB) because the surfactant solubilizes at that time and is less likely to form undesired matters causative of defects. Such surfactants, which have a characteristic of being insoluble or poorly soluble in water and soluble in alkaline developers and are polymeric surfactants, are also called hydrophobic resins, and in particular, surfactants that have high water repellency and give enhanced water slipperiness are preferred.

Specific examples of such polymeric surfactants include those containing at least one selected from repeating units represented by the following general formulae (6A) to (6E):

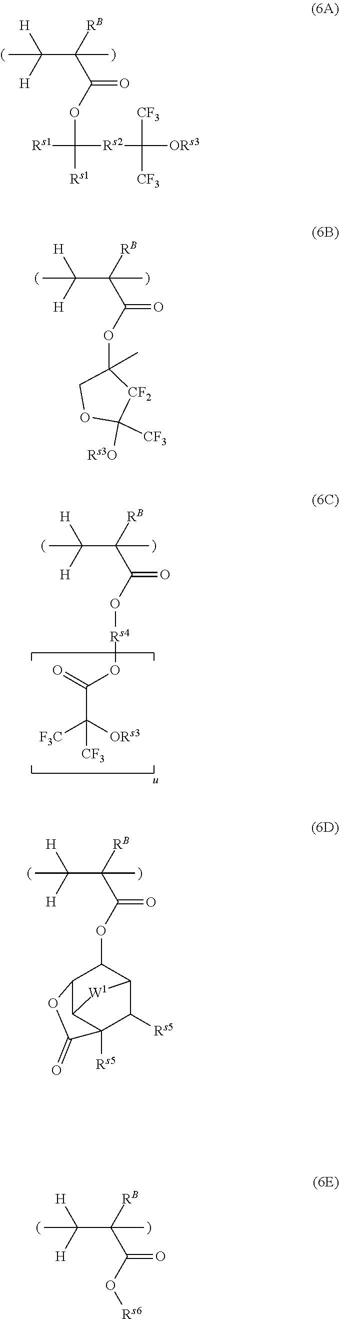

In the general formulae (6A) to (6E), R^{B} is a hydrogen atom, a fluorine atom, a methyl group, or a trifluoromethyl group. W¹ is —CH₂—, —CH₂CH₂—, —O—, or two—H atoms that separate from each other. R^{s1} groups are each independently a hydrogen atom or a hydrocarbyl group having 1 to 10 carbon atoms. R^{s2} is a single bond or a linear or branched hydrocarbylene group having 1 to 5 carbon atoms. R^{s3} groups are each independently a hydrogen atom, a hydrocarbyl group or fluorinated hydrocarbyl group having 1 to 15 carbon atoms, or an acid-unstable group. If R^{s3} is a hydrocarbyl group or fluorinated hydrocarbyl group, an ether bond or a carbonyl group is optionally present between a carbon-carbon bond. R^{s4} is a (u+1)-valent hydrocarbon group or fluorinated hydrocarbon group having 1 to 20 carbon atoms. u is 1, 2, or 3. R^{s5} groups are each independently a hydrogen atom or a group represented by —C(═O)—O—R^{sa}. R^{sa} is a fluorinated hydrocarbyl group having 1 to 20 carbon atoms. R^{s6} is a hydrocarbyl group or fluorinated hydrocarbyl group having 1 to 15 carbon atoms, wherein an ether bond or a carbonyl group is optionally present between a carbon-carbon bond therein.

The hydrocarbyl group represented by R^{s1}, having 1 to 10 carbon atoms, is preferably a saturated hydrocarbyl group, and may be linear, branched, or cyclic. Specific examples thereof include an alkyl group having 1 to 10 carbon atoms such as a methyl group, an ethyl group, a n-propyl group, an isopropyl group, a n-butyl group, an isobutyl group, a sec-butyl group, a tert-butyl group, a n-pentyl group, a n-hexyl group, a n-heptyl group, a n-octyl group, a n-nonyl group, and a n-decyl group; and a cyclic saturated hydrocarbyl group having 3 to 10 carbon atoms such as a cyclopropyl group, a cyclobutyl group, a cyclopentyl group, a cyclohexyl group, an adamantyl group, and a norbornyl group. Among them, hydrocarbyl groups having 1 to 6 carbon atoms are preferred.

The hydrocarbylene group represented by R^{s2} is preferably a saturated hydrocarbylene group, and may be linear, branched, or cyclic. Specific examples thereof include a methylene group, an ethylene group, a propylene group, a butylene group, and a pentylene group.

The hydrocarbyl group represented by R^{s3} and R^{s6} may be saturated or unsaturated, and may be linear, branched, or cyclic. Specific examples thereof include a saturated hydrocarbyl group and an aliphatic unsaturated hydrocarbyl group such as an alkenyl group and an alkynyl group, and a saturated hydrocarbyl group is preferred. Specific examples of the saturated hydrocarbyl group include not only those shown as examples of the hydrocarbyl group represented by R^{s1}, but also an undecyl group, a dodecyl group, a tridecyl group, a tetradecyl group, and a pentadecyl group. Specific examples of the fluorinated hydrocarbyl group represented by R^{s3} and R^{s6} include a group given by replacing each of some or all of the hydrogen atoms bound to the carbon atoms of the aforementioned hydrocarbyl group with a fluorine atom. As described above, an ether bond or a carbonyl group is optionally present between a carbon-carbon bond in the hydrocarbyl group.

Specific examples of the acid-unstable group represented by R^{s3} include the aforementioned groups represented by the general formulae (AL-1) to (AL-3), a trialkylsilyl group each alkyl group of which is an alkyl group having 1 to 6 carbon atoms, and an oxo group-containing alkyl group having 4 to 20 carbon atoms.

The (u+1)-valent hydrocarbon group or fluorinated hydrocarbon group represented by R^{s4} may be linear, branched, or cyclic, and specific examples thereof include a group given by further eliminating u hydrogen atoms from, for example, the aforementioned hydrocarbyl group or fluorinated hydrocarbyl group.

The fluorinated hydrocarbyl group represented by R^{sa} is preferably saturated one, and may be linear, branched, or cyclic. Specific examples thereof include a group given by replacing each of some or all of the hydrogen atoms of the hydrocarbyl group with a fluorine atom, and specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoro isopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of repeating units represented by any of the general formulae (6A) to (6E) include, but are not limited, those shown below. In the following formulae, R^{B} is as specified above:

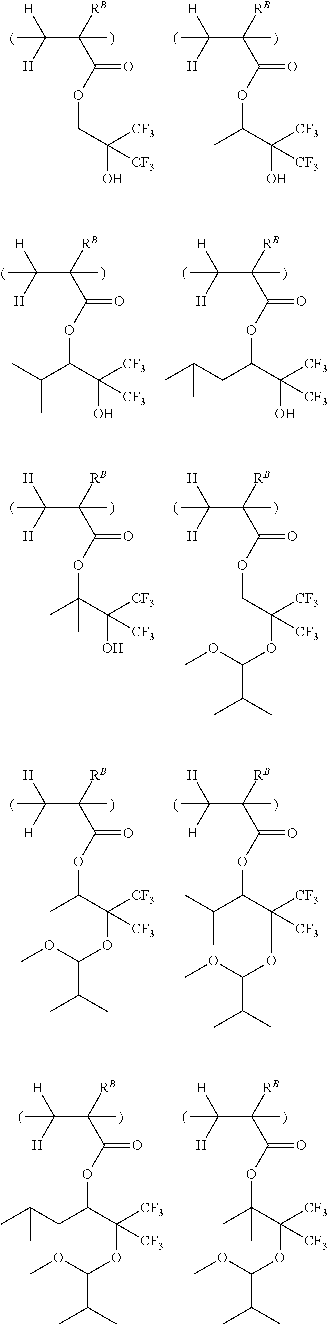

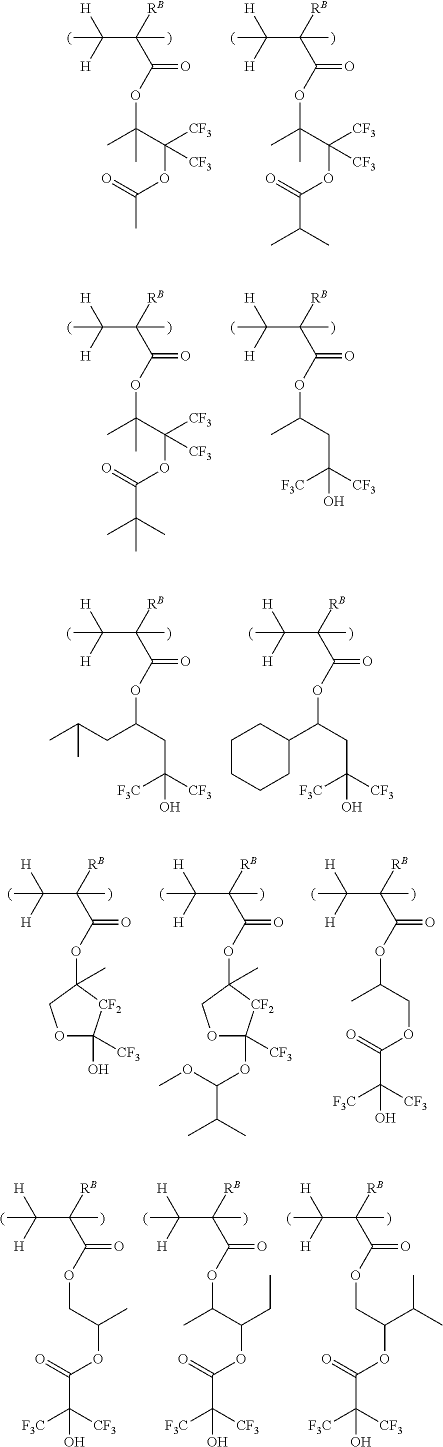

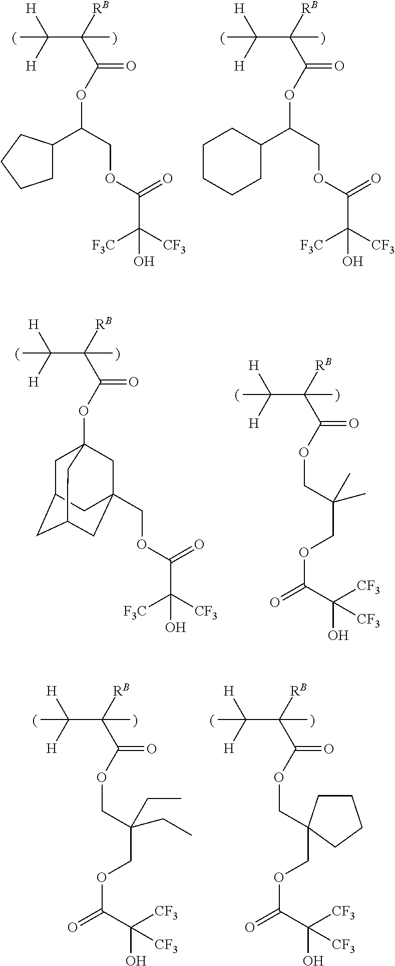

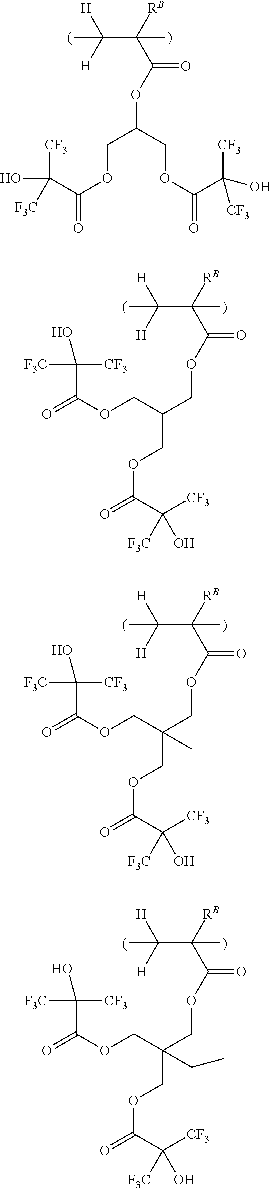

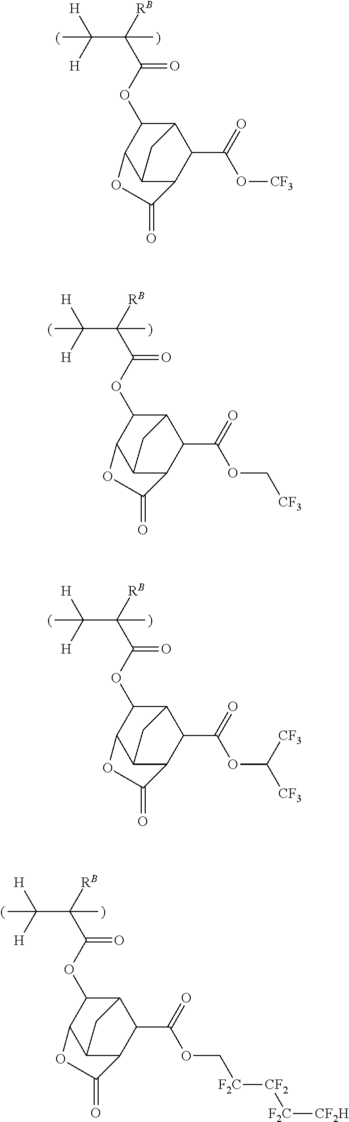

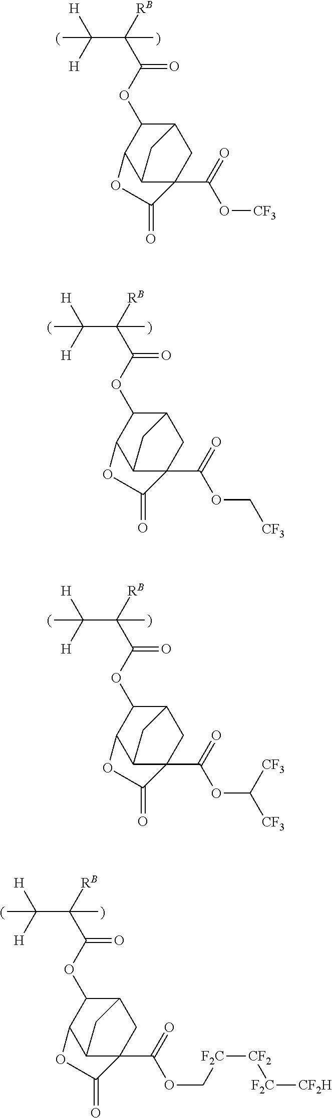

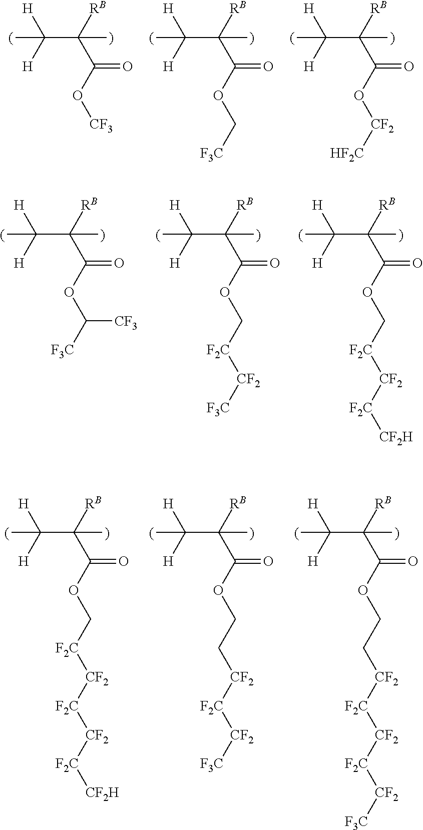

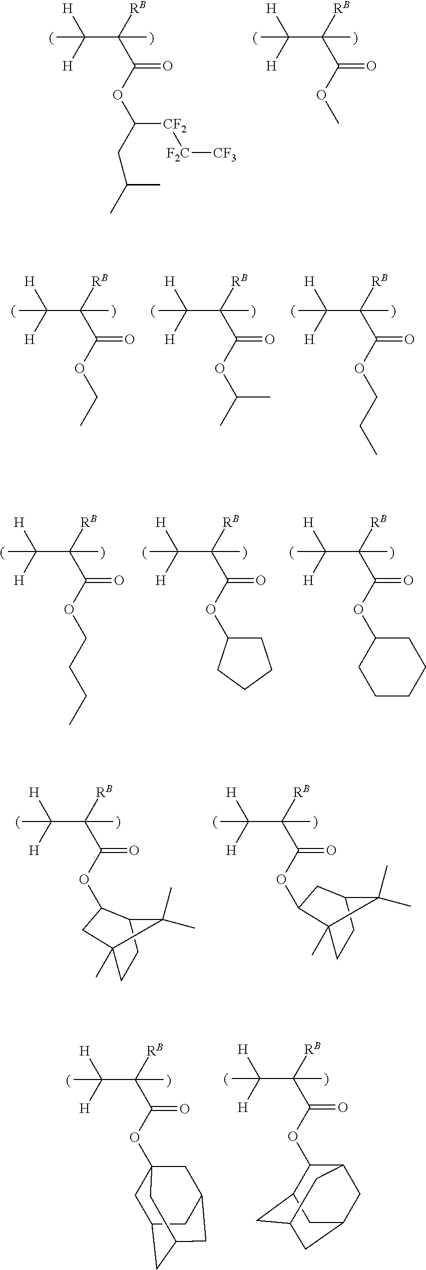

The polymeric surfactant may further contain an additional repeating unit other than the repeating units represented by the general formulae (6A) to (6E). Specific examples of the additional repeating unit include a repeating unit derived from methacrylic acid or an α-trifluoromethylacrylic acid derivative. The amount of the repeating units, contained in the polymeric surfactant, represented by the general formulae (6A) to (6E) is preferably 20 mol or more, more preferably 60 mol or more, and further preferably 100 mol to the total of the repeating units.

The Mw of the polymeric surfactant is preferably 1000 to 500000, and more preferably 3000 to 100000. The Mw/Mn is preferably 1.0 to 2.0, and more preferably 1.0 to 1.6.

Methods for synthesizing the polymeric surfactant include a method of polymerizing the repeating units represented by the general formulae (6A) to (6E) and, as necessary, monomers containing unsaturated bonds that give additional repeating units by heating in an organic solvent with addition of a radical initiator. Specific examples of the organic solvent to be used in the polymerization include toluene, benzene, THF, diethyl ether, and dioxane. Specific examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid-unstable group introduced to a monomer may be used as it is; otherwise, the acid-unstable group may be protected or partially protected after polymerization.

In the case that the polymeric surfactant is synthesized, a known chain transfer agent such as dodecylmercaptan and 2-mercaptoethanol may be used for molecular weight adjustment. In this case, the amount of the chain transfer agent to be added is preferably 0.01 to 10 mol % based on the total number of moles of the monomers to be polymerized.

If the chemically amplified resist composition of the present invention contains surfactant (E), the amount of surfactant (E) contained therein is preferably 0.1 to 50 parts by mass, and more preferably 0.5 to 10 parts by mass per 80 parts by mass of base polymer (A). With the amount of surfactant (E) contained being 0.1 parts by mass or more, sufficiently improved receding contact angle is achieved between the surface of the resist film and water; with the amount of surfactant (E) contained being 50 parts by mass or less, the surface of the resist film has low dissolution rates to developer, and the height of formed fine patterns is sufficiently maintained. For surfactant (E), one surfactant may be used alone; alternatively, two or more surfactants may be used in combination.

### [Dissolution Inhibitor (F)]

The chemically amplified resist composition of the present invention may further contain a dissolution inhibitor as component (F). In the case that the chemically amplified resist composition of the present invention is positive-type, the difference between dissolution rates in exposed parts and in unexposed parts can be more increased by blending a dissolution inhibitor, and more enhanced resolution is successfully achieved.

Specific examples of the dissolution inhibitor include a compound given by replacing each of the hydrogen atoms of phenolic hydroxy groups of a compound having a molecular weight preferably of 100 to 1000, more preferably of 150 to 800, and containing two or more phenolic hydroxy groups in the molecule with an acid-unstable group at a proportion of 0 to 100 mol % to the total, or a compound given by replacing each of the hydrogen atoms of carboxy groups of a compound containing carboxy groups in the molecule with an acid-unstable group at a proportion of 50 to 100 mol % on average to the total. Specific examples thereof include a compound given by replacing each of the hydrogen atoms of hydroxy groups or carboxy groups of bisphenol A, trisphenol, phenolphthalein, cresol-novolac, naphthalenecarboxylic acid, adamantanecarboxylic acid, or cholic acid with an acid-unstable group, for example, those shown in paragraphs [0155] to [0178] of JP 2008-122932 A.

If the chemically amplified resist composition of the present invention contains dissolution inhibitor (F), the amount of dissolution inhibitor (F) contained therein is preferably 0 to 50 parts by mass, and more preferably 5 to 40 parts by mass per 80 parts by mass of base polymer (A). For dissolution inhibitor (F), one dissolution inhibitor may be used alone; alternatively, two or more dissolution inhibitors may be used in combination.

### [Additional Component (G)]

The chemically amplified resist composition of the present invention may contain, for example, a compound that is decomposed by an acid and generates an acid (acid amplifier compound), an organic acid derivative, a fluorine-substituted alcohol, or a water repellency enhancer as additional component (G). For the acid amplifier compound, reference can be made to compounds described in JP 2009-269953 A or JP 2010-215608 A. If the acid amplifier compound is contained, the amount of the acid amplifier compound contained is preferably 0 to 5 parts by mass, and more preferably 0 to 3 parts by mass per 80 parts by mass of base polymer (A). With the amount of the acid amplifier compound contained falling within that range, acid diffusion can be controlled, and the degradation of resolution and the degradation of pattern shapes are less likely to occur. For the organic acid derivative and fluorine-substituted alcohol, reference can be made to compounds described in JP 2009-269953 A or JP 2010-215608 A.

The water repellency enhancer can be used for immersion lithography without use of any top coat. For example, the water repellency enhancer is preferably a polymer containing a fluorinated alkyl group, in particular, a polymer of specific structure containing a 1,1,1,3,3,3-hexafluoro-2-propanol residue, and more preferably those shown as examples, for example, in JP 2007-297590 A and JP 2008-111103 A. The water repellency enhancer needs to be dissolved in an alkaline developer or an organic solvent developer. The aforementioned specific water repellency enhancer having a 1,1,1,3,3,3-hexafluoro-2-propanol residue exhibits good solubility in developers. Polymers containing a repeating unit containing an amino group or an amine salt have high effects as the water repellency enhancer to inhibit the evaporation of an acid during PEB to prevent the opening failure of hole patterns after development. If the chemically amplified resist composition of the present invention contains the water repellency enhancer, the amount of the water repellency enhancer contained therein is preferably 0 to 20 parts by mass, and more preferably 0.5 to 10 parts by mass per 80 parts by mass of base polymer (A).

### [Patterning Method]

In using the chemically amplified resist composition of the present invention for production of integrated circuits of any type, known lithographic techniques can be applied. Patterning methods therewith include a method including: a step of forming a resist film on a substrate by using the chemically amplified resist composition described above; a step of exposing the resist film to a high-energy ray; and a step of developing the exposed resist film by using a developer.

First, the chemically amplified resist composition of the present invention is applied onto a substrate for integrated circuit production (e.g., Si, SiO₂, SiN, SiON, TiN, WSi, BPSG, SOG, an organic anti-reflection film) or a substrate for mask circuit production (e.g., Cr, CrO, CrON, MoSi₂, SiO₂) to give a coating film thickness of 0.01 to 2.0 μm by a proper application method such as spin coating, roll coting, flow coating, dip coating, spray coating, and doctor coating. The resultant is prebaked on a hot plate preferably at 60 to 150° C. for 10 seconds to 30 minutes, more preferably at 80 to 120° C. for 30 seconds to 20 minutes, to form a resist film.

Then, the resist film is exposed by using a high-energy ray. Examples of the high-energy ray include an ultraviolet ray, a far-ultraviolet ray, an electron beam (EB), an extreme ultraviolet ray (EUV) having a wavelength of 3 to 15 nm, an X-ray, a soft X-ray, excimer laser light, a γ-ray, and synchrotron radiation. If an ultraviolet ray, a far-ultraviolet ray, an EUV, an X-ray, a soft X-ray, excimer laser light, a γ-ray, synchrotron radiation, or the like is used as the high-energy ray, irradiation is performed directly or with use of a mask to form an intended pattern to give a dose preferably of about 1 to 200 mJ/cm², more preferably of about 10 to 100 mJ/cm². If an EB is used as the high-energy ray, drawing is performed directly or with use of a mask to form an intended pattern to give a dose preferably of about 0.1 to 100 μC/cm², more preferably of about 0.5 to 50 μC/cm². The chemically amplified resist composition of the present invention is suitable particularly for fine patterning with ArF excimer laser light having a wavelength of 193 nm, KrF excimer laser light having a wavelength of 248 nm, an EB or an EUV having a wavelength of 3 to 15 nm, an X-ray, a soft X-ray, a γ-ray, or synchrotron radiation among high-energy rays.

After the exposure, PEB may be performed on a hot plate preferably at 60 to 150° C. for 10 seconds to 30 minutes, more preferably at 80 to 120° C. for 30 seconds to 20 minutes.

After the exposure or PEB, development is performed by a conventional method such as a dip method, a puddle method, and a spray method with a developer of 0.1 to 10% by mass, preferably 2 to 5% by mass alkaline aqueous solution of tetramethylammonium hydroxide (TMAH), tetraethylammonium hydroxide, tetrapropylammonium hydroxide, tetrabutylammonium hydroxide, or the like for 3 seconds to 3 minutes, preferably for 5 seconds to 2 minutes, and as a result parts irradiated with light dissolve in the developer and unexposed parts remain undissolved, and an intended positive-type pattern is formed on the substrate.

Alternatively, a negative-type pattern can be obtained by using an organic solvent developer in place of the alkaline aqueous solution. Specific examples of the developer to be used in this case include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate. One of these organic solvents may be used alone; alternatively, two or more thereof may be used as a mixture.

At the completion of development, rinsing may be performed. Preferred as the rinsing solution is a solvent that is miscible with the developer and does not dissolve the resist film. For the solvent, an alcohol having 3 to 10 carbon atoms, an ether compound having 8 to 12 carbon atoms, an alkane, alkene, or alkyne having 6 to 12 carbon atoms, or an aromatic solvent is preferably used.

Specific examples of the alcohol having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol.

Specific examples of the ether compound having 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether.

Specific examples of the alkane having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Specific examples of the alkene having 6 to 12 carbon atoms include hexene, heptene, octene, cyclohexene, methylcyclohexene, dimethylcyclohexene, cycloheptene, and cyclooctene. Specific examples of the alkyne having 6 to 12 carbon atoms include hexyne, heptyne, and octyne.

Specific examples of the aromatic solvent include toluene, xylene, ethylbenzene, isopropylbenzene, tert-butylbenzene, and mesitylene.

The generation of collapse and defects of resist patterns can be reduced by rinsing. The rinsing is not necessarily essential, and the amount of usage of the solvent can be reduced by omitting rinsing.

Hole patterns and trench patterns after development can be shrunk by thermal flow, a RELACS technique, or a DSA technique. If a shrinking agent is applied onto a hole pattern, the diffusion of an acid catalyst from the resist film during bake causes the shrinking agent to crosslink on the surface of the resist film, and the shrinking agent attaches to the side walls of the hole pattern. The bake temperature is preferably 70 to 180° C., and more preferably 80 to 170° C., and the bake time is preferably 10 to 300 seconds; thereby, excessive portions of the shrinking agent are removed, and the hole pattern is down-sized.

### EXAMPLES

The following specifically describes the present invention by showing examples and comparative examples, but the present invention is not limited by the following examples. An apparatus used is as follows:
MALDI TOF-MS: S3000 manufactured by JEOL Ltd.

### [1] Synthesis of Sulfonium Salt Monomer

### [Example 1-1] Synthesis of Sulfonium Salt Monomer a-1

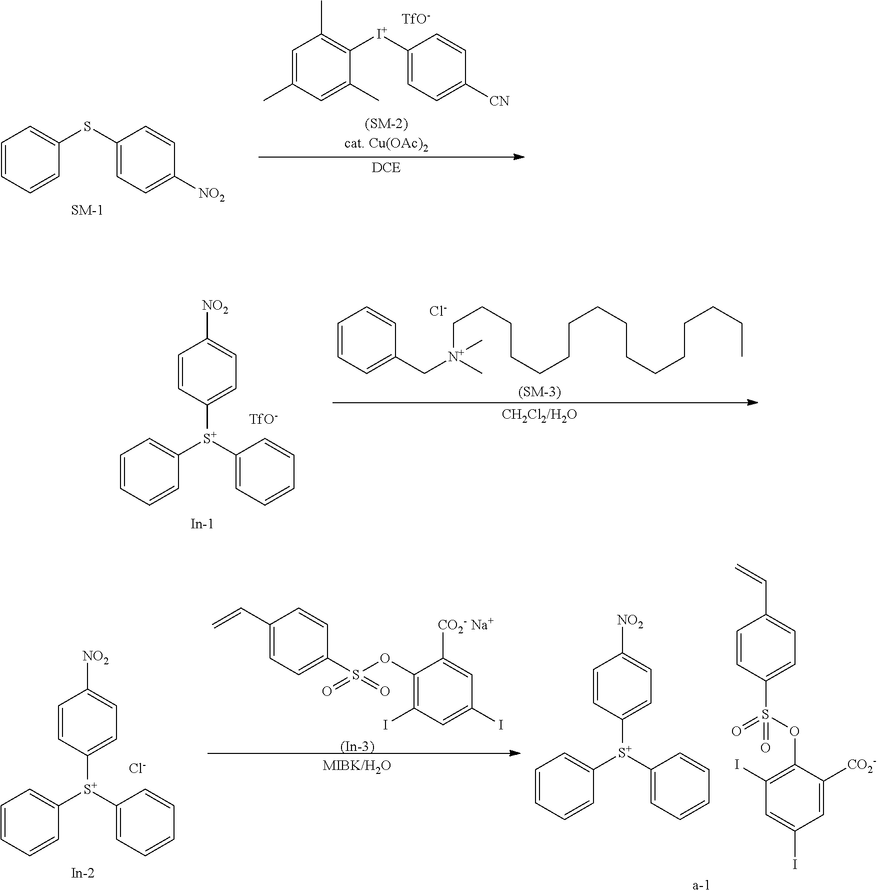

### (1) Synthesis of Intermediate In-1

Under a nitrogen atmosphere, SM-1 (34.7 g), SM-2 (85.0 g), and copper acetate (8.24 g) were dissolved in dichloroethane (300 g). Thereafter, the reaction system was heated to 100° C., and aged for 15 hours. After the aging, the reaction system was cooled, and water (150 g) was added to terminate the reaction. After that, the organic layer was taken by separation and washed with water, and then concentrated under reduced pressure to distill off the solvent. The residue was recrystallized with diisopropyl ether to afford 42.5 g of intermediate In-1 as yellow crystals (% yield: 62%).

### (2) Synthesis of Intermediate In-2

Under a nitrogen atmosphere, intermediate In-1 (42.5 g) and SM-3 (36.8 g) were dissolved in a mixed solution of methylene chloride (300 g) and water (300 g). The aqueous layer was taken by separation, and further washed with diisopropyl ether (100 g), giving 328.6 g of an aqueous solution of intermediate In-2 (% yield: 99%).

### (3) Synthesis of Sulfonium Salt Monomer a-1

Under a nitrogen atmosphere, the aqueous solution of intermediate In-2 (267.6 g), intermediate In-3 (43.4 g), methyl isobutyl ketone (300 g), and water (150 g) were put, and stirred at room temperature for 30 minutes. The organic layer was taken by separation, washed with water, and then concentrated under reduced pressure. The residue was washed with diisopropyl ether, and concentrated to afford 60.2 g of a-1 as an oily substance (% yield: 93%).

The following shows a result of TOF-MS for sulfonium salt monomer a-1.

### MALDI TOF-MS:

POSITIVE M+288 (corresponding to C₁₉H₁₄NS⁺)
NEGATIVE M-555 (corresponding to C₁₅H₉I₂O₅S⁻)

### [Examples 1-2 to 1-7] Synthesis of Sulfonium Salt Monomers a-2 to a-7

Sulfonium salt monomers a-2 to a-7 represented by the following formulae were synthesized by using the corresponding raw materials and known organic synthesis reactions:

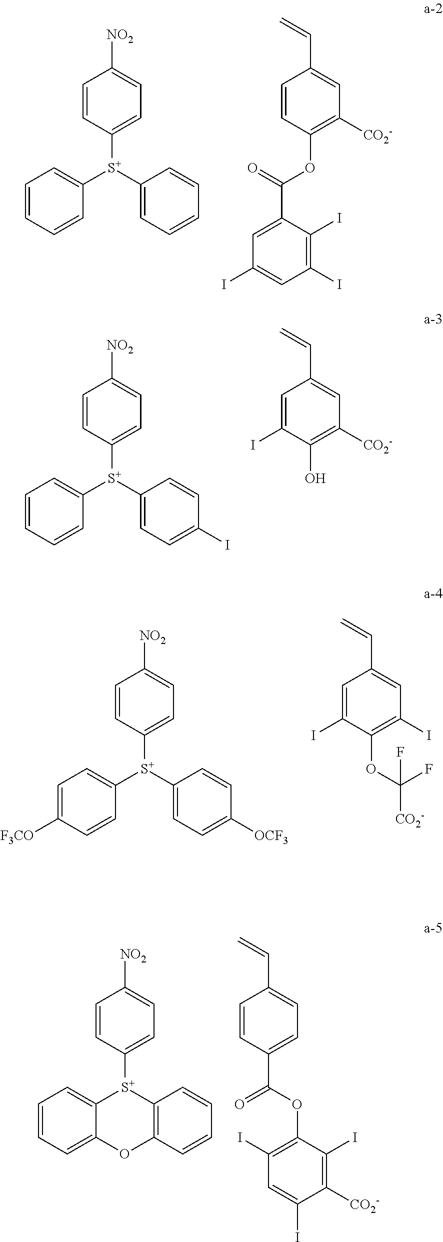

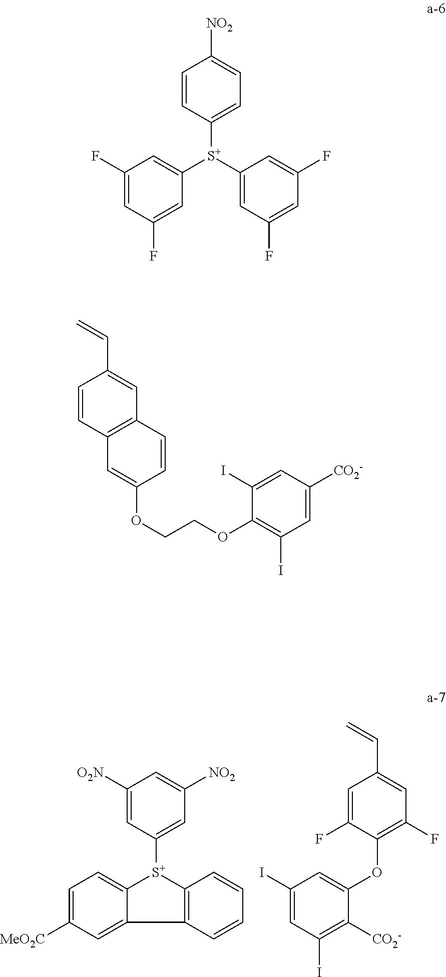

### [Comparative Examples 1-1 to 1-5] Synthesis of Comparative Onium Salt Monomers Ca-1 to Ca-5

Comparative onium salt monomers ca-1 to ca-5 represented by the following formulae were synthesized by using the corresponding raw materials and known organic synthesis reactions:

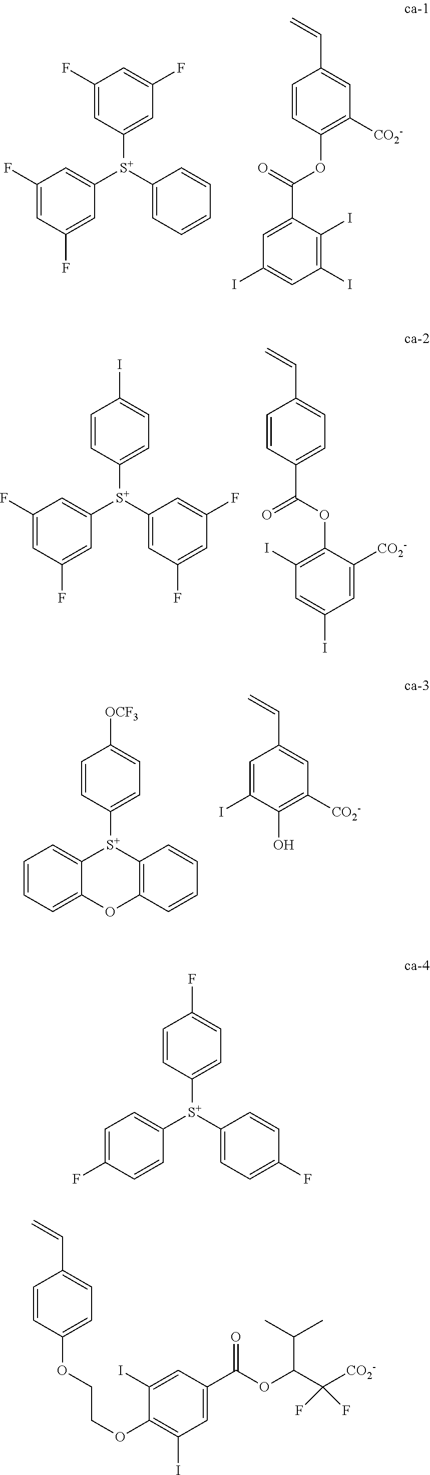

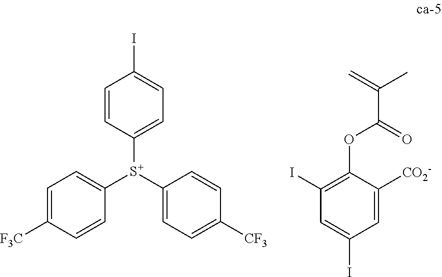

### [2] Synthesis of Base Polymer

Among monomers used for synthesis of base polymers, those other than monomers a-1 to a-7 and comparative monomers ca-1 to ca-5 are as follows:

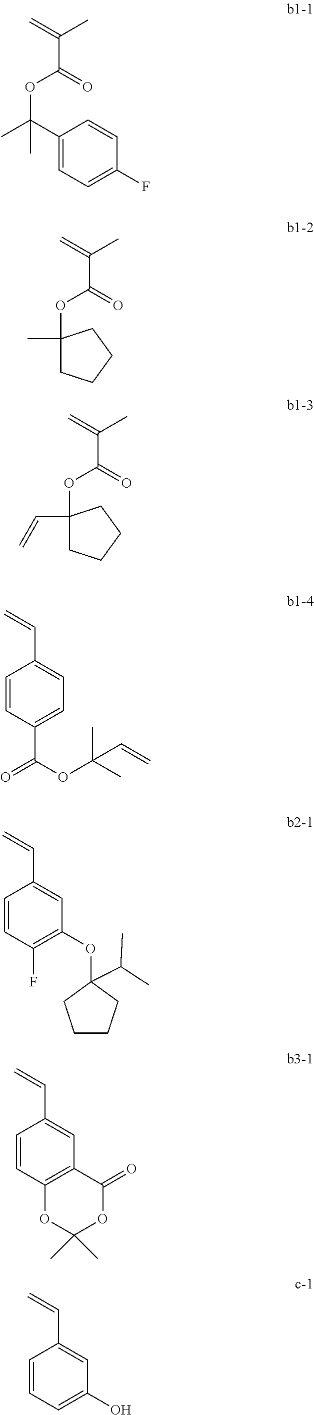

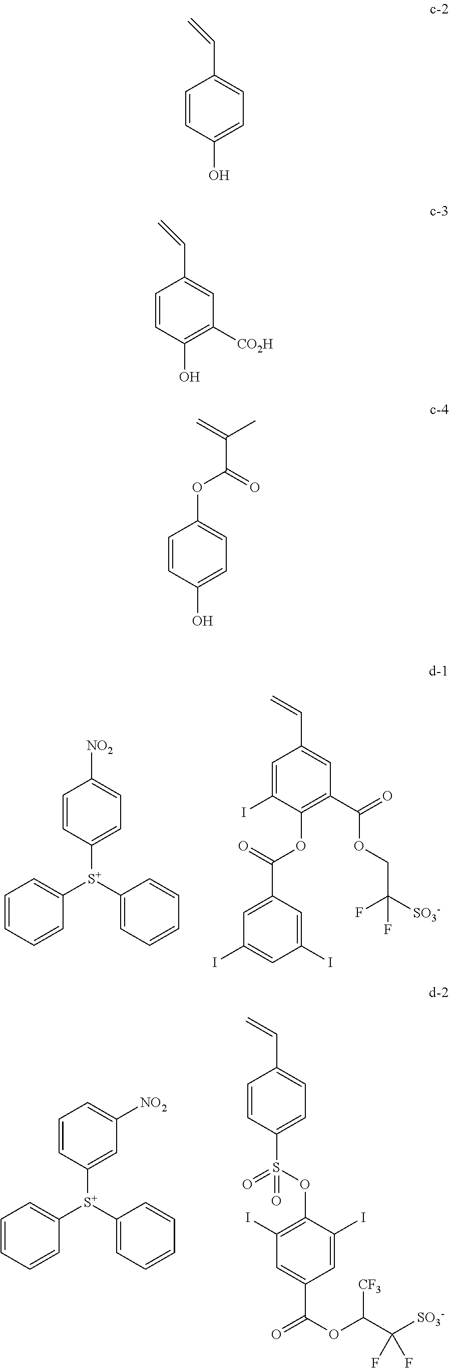

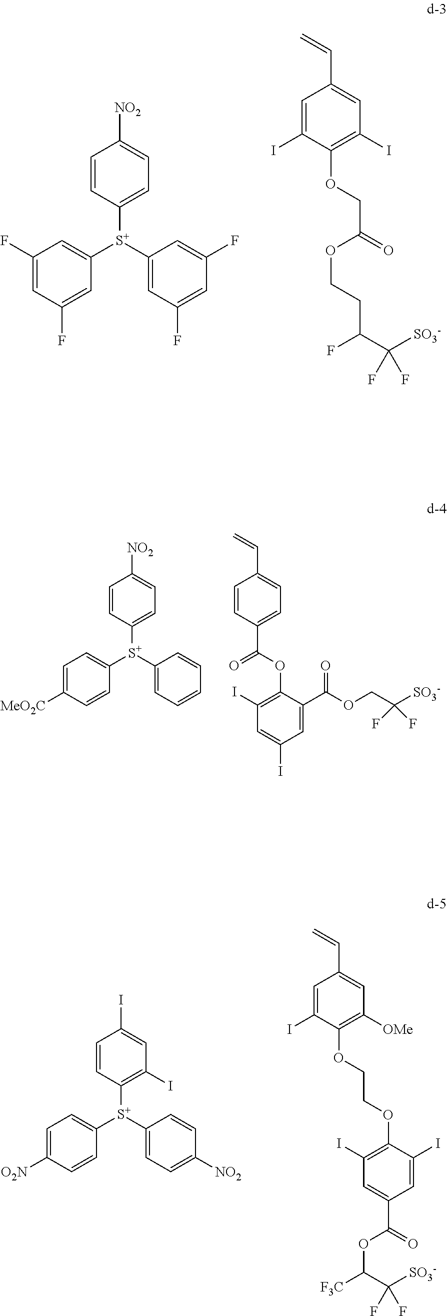

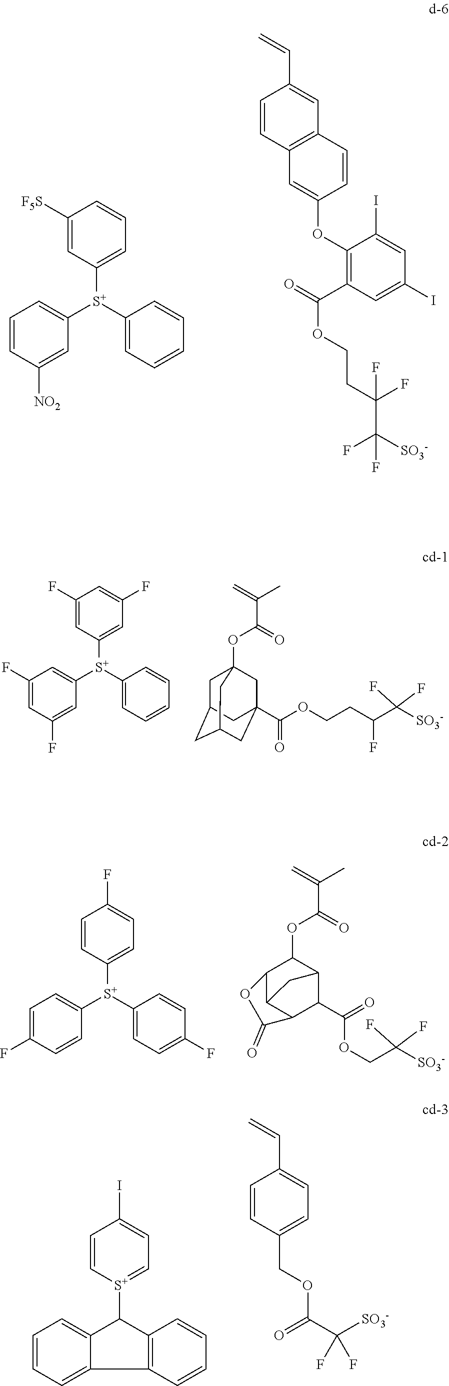

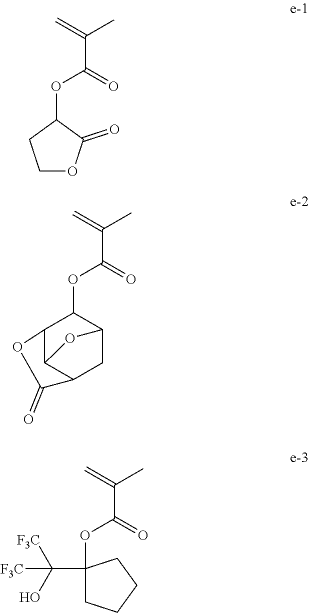

### [Example 2-1] Synthesis of Polymer P-1

Under a nitrogen atmosphere, monomer a-1 (6.1 g), monomer b1-1 (15.7 g), monomer c-1 (5.1 g), monomer d-1 (23.2 g), 1.62 g of V-601 (manufactured by FUJIFILM Wako Pure Chemical Corporation), and 70 g of MEK were put in a flask to prepare a monomer-polymerization initiator solution. In another flask under a nitrogen atmosphere, 23 g of MEK was put and heated to 80° C. with stirring, and then the monomer-polymerization initiator solution was added thereto dropwise over 4 hours. After completion of the dropwise addition, stirring was continued for 2 hours while the temperature of the polymerization solution was kept at 80° C., and the resultant was then cooled to room temperature. The resulting polymerization solution was added dropwise to 1500 g of hexane vigorously stirred, and a polymer precipitated was separated by filtration. The resulting polymer was washed twice with 300 g of hexane, and then vacuum-dried at 50° C. for 20 hours to afford polymer P-1 in the form of white powder (yield: 96.1 g, % yield: 96%). The Mw and Mw/Mn of polymer P-1 were 10400 and 1.61, respectively. The Mw is a measurement in terms of polystyrene as determined by GPC with use of DMF as a solvent.

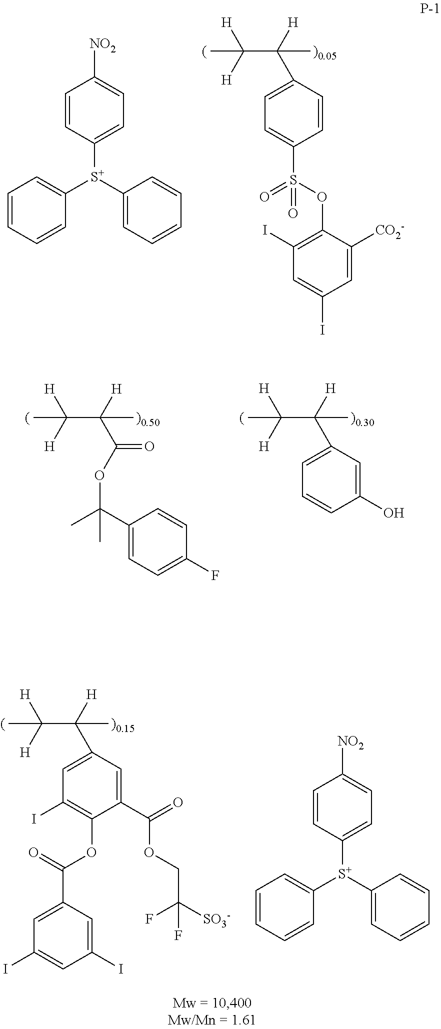

### Examples 2-2 to 2-50, Comparative Examples 2-1 to 2-38

### Synthesis of Polymers P-2 to P-50 and Comparative Polymers CP-1 to CP-38

Polymers shown in Tables 1 to 3 were produced in the same manner as in Synthesis Example 2-1 except that the types and blend ratios of monomers were changed.

TABLE 1

| | | Introduction | | Introduction | | Introduction | | Introduction | | Introduction | | |
|---|---|---|---|---|---|---|---|---|---|---|---|---|
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-1 | a-1 | 5 | b1-1 | 50 | c-1 | 30 | d-1 | 15 | — | — | 10400 | 1.61 |
| P-2 | a-2 | 5 | b1-1 | 50 | c-1 | 30 | d-1 | 15 | — | — | 10100 | 1.62 |
| P-3 | a-3 | 5 | b1-1 | 50 | c-1 | 30 | d-1 | 15 | — | — | 10300 | 1.61 |
| P-4 | a-4 | 5 | b1-1 | 50 | c-1 | 30 | d-1 | 15 | — | — | 10200 | 1.61 |
| P-5 | a-5 | 5 | b1-1 | 50 | c-1 | 30 | d-1 | 15 | — | — | 10400 | 1.62 |
| P-6 | a-6 | 5 | b1-1 | 50 | c-1 | 30 | d-1 | 15 | — | — | 10300 | 1.63 |
| P-7 | a-7 | 5 | b1-1 | 50 | c-1 | 30 | d-1 | 15 | — | — | 10300 | 1.62 |
| P-8 | a-1 | 5 | b1-2 | 50 | c-1 | 30 | d-1 | 15 | — | — | 10200 | 1.61 |
| P-9 | a-2 | 5 | b1-2 | 50 | c-1 | 30 | d-1 | 15 | — | — | 10200 | 1.61 |
| P-10 | a-3 | 5 | b1-2 | 50 | c-1 | 30 | d-1 | 15 | — | — | 10200 | 1.6 |
| P-11 | a-4 | 5 | b1-2 | 50 | c-1 | 30 | d-1 | 15 | — | — | 10300 | 1.62 |
| P-12 | a-5 | 5 | b1-2 | 50 | c-1 | 30 | d-1 | 15 | — | — | 10400 | 1.63 |
| P-13 | a-6 | 5 | b1-2 | 50 | c-1 | 30 | d-1 | 15 | — | — | 10400 | 1.6 |
| P-14 | a-7 | 5 | b1-2 | 50 | c-1 | 30 | d-1 | 15 | — | — | 10200 | 1.61 |
| P-15 | a-1 | 5 | b1-3 | 50 | c-1 | 30 | d-1 | 15 | — | — | 10300 | 1.62 |
| P-16 | a-2 | 5 | b1-3 | 50 | c-1 | 30 | d-1 | 15 | — | — | 10400 | 1.61 |
| P-17 | a-3 | 5 | b1-3 | 50 | c-1 | 30 | d-1 | 15 | — | — | 10200 | 1.62 |
| P-18 | a-4 | 5 | b1-3 | 50 | c-1 | 30 | d-1 | 15 | — | — | 10400 | 1.60 |
| P-19 | a-5 | 5 | b1-3 | 50 | c-1 | 30 | d-1 | 15 | — | — | 10200 | 1.63 |
| P-20 | a-6 | 5 | b1-3 | 50 | c-1 | 30 | d-1 | 15 | — | — | 10200 | 1.62 |
| P-21 | a-7 | 5 | b1-3 | 50 | c-1 | 30 | d-1 | 15 | — | — | 10300 | 1.62 |
| P-22 | a-1 | 5 | b1-4 | 50 | c-1 | 30 | d-1 | 15 | — | — | 10300 | 1.61 |
| P-23 | a-2 | 5 | b1-4 | 50 | c-1 | 30 | d-1 | 15 | — | — | 10400 | 1.61 |
| P-24 | a-3 | 5 | b1-4 | 50 | c-1 | 30 | d-1 | 15 | — | — | 10100 | 1.64 |
| P-25 | a-4 | 5 | b1-4 | 50 | c-1 | 30 | d-1 | 15 | — | — | 10200 | 1.62 |
| P-26 | a-5 | 5 | b1-4 | 50 | c-1 | 30 | d-1 | 15 | — | — | 10500 | 1.63 |
| P-27 | a-6 | 5 | b1-4 | 50 | c-1 | 30 | d-1 | 15 | — | — | 10300 | 1.62 |
| P-28 | a-7 | 5 | b1-4 | 50 | c-1 | 30 | d-1 | 15 | — | — | 10400 | 1.61 |
| P-29 | a-1 | 5 | b1-1 | 25 | b2-1 | 25 | c-2 | 30 | d-1 | 15 | 10300 | 1.61 |
| P-30 | a-1 | 5 | b1-2 | 25 | b2-1 | 25 | c-2 | 30 | d-1 | 15 | 10100 | 1.62 |

TABLE 2

| | | Introduction | | Introduction | | Introduction | | Introduction | | Introduction | | |
|---|---|---|---|---|---|---|---|---|---|---|---|---|
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| P-31 | a-1 | 5 | b1-3 | 25 | b2-1 | 25 | c-2 | 30 | d-1 | 8 | 10300 | 1.62 |
| P-32 | a-1 | 5 | b1-3 | 30 | b2-1 | 25 | c-3 | 25 | d-1 | 15 | 10100 | 1.61 |
| P-33 | a-1 | 5 | b1-1 | 25 | b2-1 | 25 | c-4 | 30 | d-2 | 15 | 10200 | 1.62 |
| P-34 | a-2 | 5 | b1-1 | 30 | b3-1 | 15 | c-1 | 30 | d-2 | 15 | 10300 | 1.63 |
| P-35 | a-2 | 5 | b1-1 | 25 | b1-4 | 25 | c-2 | 30 | d-1 | 15 | 10200 | 1.62 |
| P-36 | a-1 | 5 | b1-1 | 50 | c-1 | 30 | d-2 | 15 | — | — | 10300 | 1.61 |
| P-37 | a-1 | 5 | b1-1 | 50 | c-1 | 30 | d-3 | 15 | — | — | 10100 | 1.61 |
| P-38 | a-2 | 5 | b1-1 | 50 | c-1 | 30 | d-4 | 15 | — | — | 10100 | 1.62 |
| P-39 | a-3 | 5 | b1-1 | 50 | c-1 | 30 | d-5 | 15 | — | — | 10200 | 1.63 |
| P-40 | a-4 | 5 | b1-1 | 50 | c-1 | 30 | d-6 | 15 | — | — | 10300 | 1.62 |
| P-41 | a-2 | 5 | b1-1 | 45 | c-2 | 25 | d-1 | 15 | e-1 | 10 | 10400 | 1.60 |
| P-42 | a-3 | 5 | b1-3 | 45 | c-4 | 25 | d-2 | 15 | e-2 | 10 | 10500 | 1.62 |
| P-43 | a-4 | 5 | b1-3 | 45 | c-2 | 25 | d-3 | 10 | e-3 | 5 | 10300 | 1.61 |
| P-44 | a-1 | 7 | b1-1 | 58 | c-1 | 35 | — | — | — | — | 10200 | 1.63 |
| P-45 | a-2 | 7 | b1-1 | 58 | c-1 | 35 | — | — | — | — | 10300 | 1.64 |
| P-46 | a-3 | 7 | b1-2 | 58 | c-2 | 35 | — | — | — | — | 10200 | 1.62 |
| P-47 | a-4 | 7 | b1-3 | 58 | c-3 | 35 | — | — | — | — | 10100 | 1.63 |
| P-48 | a-5 | 7 | b1-4 | 58 | c-4 | 35 | — | — | — | — | 10200 | 1.64 |
| P-49 | a-6 | 7 | b1-1 | 29 | b2-1 | 29 | c-1 | 35 | — | — | 10100 | 1.64 |
| P-50 | a-7 | 7 | b1-1 | 29 | b3-1 | 29 | c-2 | 35 | — | — | 10200 | 1.62 |

TABLE 3

| | | Introduction | | Introduction | | Introduction | | Introduction | | Introduction | | |
|---|---|---|---|---|---|---|---|---|---|---|---|---|
| | | ratio | | ratio | | ratio | | ratio | | ratio | | |
| Polymer | Unit 1 | (mol %) | Unit 2 | (mol %) | Unit 3 | (mol %) | Unit 4 | (mol %) | Unit 5 | (mol %) | Mw | Mw/Mn |
| CP-1 | ca-1 | 5 | b1-1 | 50 | c-1 | 30 | d-1 | 15 | — | — | 10200 | 1.61 |
| CP-2 | ca-2 | 5 | b1-1 | 50 | c-1 | 30 | d-1 | 15 | — | — | 10200 | 1.62 |
| CP-3 | ca-3 | 5 | b1-1 | 50 | c-1 | 30 | d-1 | 15 | — | — | 10500 | 1.64 |
| CP-4 | ca-4 | 5 | b1-1 | 50 | c-1 | 30 | d-1 | 15 | — | — | 10300 | 1.63 |
| CP-5 | ca-5 | 5 | b1-1 | 50 | c-1 | 30 | d-1 | 15 | — | — | 10400 | 1.62 |
| CP-6 | ca-1 | 5 | b1-2 | 50 | c-1 | 30 | d-1 | 15 | — | — | 10300 | 1.61 |
| CP-7 | ca-2 | 5 | b1-2 | 50 | c-1 | 30 | d-1 | 15 | — | — | 10200 | 1.62 |
| CP-8 | ca-3 | 5 | b1-2 | 50 | c-1 | 30 | d-1 | 15 | — | — | 10600 | 1.60 |
| CP-9 | ca-4 | 5 | b1-2 | 50 | c-1 | 30 | d-1 | 15 | — | — | 10800 | 1.61 |
| CP-10 | ca-5 | 5 | b1-2 | 50 | c-1 | 30 | d-1 | 15 | — | — | 10500 | 1.62 |
| CP-11 | ca-1 | 5 | b1-3 | 50 | c-1 | 30 | d-1 | 15 | — | — | 10400 | 1.63 |
| CP-12 | ca-2 | 5 | b1-3 | 50 | c-1 | 30 | d-1 | 15 | — | — | 10300 | 1.61 |
| CP-13 | ca-3 | 5 | b1-3 | 50 | c-1 | 30 | d-1 | 15 | — | — | 10600 | 1.62 |
| CP-14 | ca-4 | 5 | b1-3 | 50 | c-1 | 30 | d-1 | 15 | — | — | 10100 | 1.63 |
| CP-15 | ca-5 | 5 | b1-3 | 50 | c-1 | 30 | d-1 | 15 | — | — | 10200 | 1.61 |
| CP-16 | ca-1 | 5 | b1-4 | 50 | c-1 | 30 | d-1 | 15 | — | — | 10400 | 1.63 |
| CP-17 | ca-2 | 5 | b1-4 | 50 | c-1 | 30 | d-1 | 15 | — | — | 10300 | 1.65 |
| CP-18 | ca-3 | 5 | b1-4 | 50 | c-1 | 30 | d-1 | 15 | — | — | 10400 | 1.62 |
| CP-19 | ca-4 | 5 | b1-4 | 50 | c-1 | 30 | d-1 | 15 | — | — | 10600 | 1.62 |
| CP-20 | ca-5 | 5 | b1-4 | 50 | c-1 | 30 | d-1 | 15 | — | — | 10100 | 1.61 |
| CP-21 | ca-1 | 5 | b1-1 | 25 | b2-1 | 25 | c-2 | 30 | d-1 | 15 | 10200 | 1.62 |
| CP-22 | ca-2 | 5 | b1-2 | 25 | b2-1 | 25 | c-2 | 30 | d-2 | 15 | 10000 | 1.63 |
| CP-23 | ca-4 | 5 | b1-3 | 25 | b2-1 | 25 | c-2 | 30 | d-4 | 8 | 10300 | 1.64 |
| CP-24 | ca-1 | 5 | b1-1 | 25 | b2-1 | 25 | c-2 | 30 | cd-1 | 15 | 10200 | 1.62 |
| CP-25 | ca-2 | 5 | b1-2 | 25 | b2-1 | 25 | c-2 | 30 | cd-2 | 15 | 10600 | 1.61 |
| CP-26 | ca-5 | 5 | b1-3 | 25 | b2-1 | 25 | c-2 | 30 | cd-3 | 15 | 10300 | 1.62 |
| CP-27 | ca-1 | 5 | b1-1 | 50 | c-1 | 30 | cd-1 | 15 | — | — | 10100 | 1.63 |
| CP-28 | ca-1 | 5 | b1-1 | 50 | c-1 | 30 | cd-2 | 15 | — | — | 10300 | 1.62 |
| CP-29 | ca-2 | 5 | b1-1 | 50 | c-1 | 30 | cd-3 | 15 | — | — | 10200 | 1.61 |
| CP-30 | ca-1 | 5 | b1-1 | 45 | c-2 | 25 | d-1 | 15 | e-1 | 10 | 10400 | 1.63 |
| CP-31 | ca-2 | 5 | b1-3 | 45 | c-4 | 25 | cd-1 | 15 | e-2 | 10 | 10500 | 1.63 |
| CP-32 | ca-4 | 5 | b1-3 | 45 | c-2 | 25 | cd-2 | 10 | e-3 | 5 | 10300 | 1.62 |
| CP-33 | ca-1 | 7 | b1-1 | 58 | c-1 | 35 | — | — | — | — | 10200 | 1.61 |
| CP-34 | ca-2 | 7 | b1-1 | 58 | c-2 | 35 | — | — | — | — | 10200 | 1.63 |
| CP-35 | ca-4 | 7 | b1-2 | 58 | c-2 | 35 | — | — | — | — | 10400 | 1.62 |
| CP-36 | ca-1 | 7 | b1-1 | 29 | b2-1 | 29 | c-1 | 35 | — | — | 10600 | 1.65 |
| CP-37 | b1-1 | 55 | c-1 | 30 | cd-1 | 15 | — | — | — | — | 10300 | 1.61 |
| CP-38 | b1-2 | 50 | c-2 | 50 | — | — | — | — | — | — | 10100 | 1.64 |

### [3]Preparation of Chemically Amplified Resist Compositions

### Examples 3-1 to 3-53, Comparative Examples 3-1 to 3-40

Specific components selected from the base polymers of the present invention (P-1 to P-50), comparative base polymers (CP-1 to CP-38), acid generators (PAG-1, PAG-2), and quenchers (SQ-1, AQ-1) at a composition listed in Tables 4 to 6 shown below were dissolved in a solvent containing 0.01% by mass of FC-4430 manufactured by 3M Company as a surfactant to prepare a solution, and the solution was filtered through a 0.2-μm Teflon (registered trademark) filter to prepare chemically amplified resist compositions (R-i to R-53, CR-i to CR-40).

TABLE 4

| | | | | | | Solvent 2 | Solvent 3 |
|---|---|---|---|---|---|---|---|
| | Resist | Base polymer | Quencher | Acid generator | Solvent | (part by | (part by |
| | composition | (part by mass) | (part by mass) | (part by mass) | (part by mass) | mass) | mass) |
| Example 3-1 | R-1 | P-1(80) | — | — | PGMEA(2250) | EL(2800) | DAA(550) |
| Example 3-2 | R-2 | P-2(80) | — | — | PGMEA(2250) | EL(2800) | DAA(550) |
| Example 3-3 | R-3 | P-3(80) | — | — | PGMEA(2250) | EL(2800) | DAA(550) |
| Example 3-4 | R-4 | P-4(80) | — | — | PGMEA(2250) | EL(2800) | DAA(550) |
| Example 3-5 | R-5 | P-5(80) | — | — | PGMEA(2250) | EL(2800) | DAA(550) |
| Example 3-6 | R-6 | P-6(80) | — | — | PGMEA(2250) | EL(2800) | DAA(550) |
| Example 3-7 | R-7 | P-7(80) | — | — | PGMEA(2250) | EL(2800) | DAA(550) |
| Example 3-8 | R-8 | P-8(80) | — | — | PGMEA(2250) | EL(2800) | DAA(550) |
| Example 3-9 | R-9 | P-9(80) | — | — | PGMEA(2250) | EL(2800) | DAA(550) |
| Example 3-10 | R-10 | P-10(80) | — | — | PGMEA(2250) | EL(2800) | DAA(550) |
| Example 3-11 | R-11 | P-11(80) | — | — | PGMEA(2250) | EL(2800) | DAA(550) |
| Example 3-12 | R-12 | P-12(80) | — | — | PGMEA(2250) | EL(2800) | DAA(550) |
| Example 3-13 | R-13 | P-13(80) | — | — | PGMEA(2250) | EL(2800) | DAA(550) |
| Example 3-14 | R-14 | P-14(80) | — | — | PGMEA(2250) | EL(2800) | DAA(550) |
| Example 3-15 | R-15 | P-15(80) | — | — | PGMEA(2250) | EL(2800) | DAA(550) |
| Example 3-16 | R-16 | P-16(80) | — | — | PGMEA(2250) | EL(2800) | DAA(550) |
| Example 3-17 | R-17 | P-17(80) | — | — | PGMEA(2250) | EL(2800) | DAA(550) |
| Example 3-18 | R-18 | P-18(80) | — | — | PGMEA(2250) | EL(2800) | DAA(550) |
| Example 3-19 | R-19 | P-19(80) | — | — | PGMEA(2250) | EL(2800) | DAA(550) |
| Example 3-20 | R-20 | P-20(80) | — | — | PGMEA(2250) | EL(2800) | DAA(550) |
| Example 3-21 | R-21 | P-21(80) | — | — | PGMEA(2250) | EL(2800) | DAA(550) |
| Example 3-22 | R-22 | P-22(80) | — | — | PGMEA(2250) | EL(2800) | DAA(550) |
| Example 3-23 | R-23 | P-23(80) | — | — | PGMEA(2250) | EL(2800) | DAA(550) |
| Example 3-24 | R-24 | P-24(80) | — | — | PGMEA(2250) | EL(2800) | DAA(550) |
| Example 3-25 | R-25 | P-25(80) | — | — | PGMEA(2250) | EL(2800) | DAA(550) |
| Example 3-26 | R-26 | P-26(80) | — | — | PGMEA(2250) | EL(2800) | DAA(550) |
| Example 3-27 | R-27 | P-27(80) | — | — | PGMEA(2250) | EL(2800) | DAA(550) |
| Example 3-28 | R-28 | P-28(80) | — | — | PGMEA(2250) | EL(2800) | DAA(550) |
| Example 3-29 | R-29 | P-29(80) | — | — | PGMEA(2250) | EL(2800) | DAA(550) |
| Example 3-30 | R-30 | P-30(80) | — | — | PGMEA(2250) | EL(2800) | DAA(550) |

TABLE 5

| | | | Quencher | | | Solvent 2 | Solvent 3 |
|---|---|---|---|---|---|---|---|
| | Resist | Base polymer | (part by | Acid generator | Solvent | (part by | (part by |
| | composition | (part by mass) | mass) | (part by mass) | (part by mass) | mass) | mass) |
| Example 3-31 | R-31 | P-31(80) | — | PAG-1(10) | PGMEA(2250) | EL(2800) | DAA(550) |
| Example 3-32 | R-32 | P-32(80) | — | — | PGMEA(2250) | EL(2800) | DAA(550) |
| Example 3-33 | R-33 | P-33(80) | — | — | PGMEA(2250) | EL(2800) | DAA(550) |
| Example 3-34 | R-34 | P-34(80) | — | — | PGMEA(2250) | EL(2800) | DAA(550) |
| Example 3-35 | R-35 | P-35(80) | — | — | PGMEA(2250) | EL(2800) | DAA(550) |
| Example 3-36 | R-36 | P-36(80) | — | — | PGMEA(2250) | EL(2800) | DAA(550) |
| Example 3-37 | R-37 | P-37(80) | — | — | PGMEA(2250) | EL(2800) | DAA(550) |
| Example 3-38 | R-38 | P-38(80) | — | — | PGMEA(2250) | EL(2800) | DAA(550) |
| Example 3-39 | R-39 | P-39(80) | — | — | PGMEA(2250) | EL(2800) | DAA(550) |
| Example 3-40 | R-40 | P-40(80) | — | — | PGMEA(2250) | EL(2800) | DAA(550) |
| Example 3-41 | R-41 | P-41(80) | — | — | PGMEA(2250) | EL(2800) | DAA(550) |
| Example 3-42 | R-42 | P-42(80) | — | — | PGMEA(2250) | EL(2800) | DAA(550) |
| Example 3-43 | R-43 | P-43(80) | — | PAG-2(10) | PGMEA(2250) | EL(2800) | DAA(550) |
| Example 3-44 | R-44 | P-44(80) | — | PAG-1(10) | PGMEA(2250) | EL(2800) | DAA(550) |
| Example 3-45 | R-45 | P-45(80) | — | PAG-1(10) | PGMEA(2250) | EL(2800) | DAA(550) |
| Example 3-46 | R-46 | P-46(80) | — | PAG-1(10) | PGMEA(2250) | EL(2800) | DAA(550) |
| Example 3-47 | R-47 | P-47(80) | — | PAG-2(10) | PGMEA(2250) | EL(2800) | DAA(550) |
| Example 3-48 | R-48 | P-48(80) | — | PAG-1(10) | PGMEA(2250) | EL(2800) | DAA(550) |
| Example 3-49 | R-49 | P-49(80) | — | PAG-2(10) | PGMEA(2250) | EL(2800) | DAA(550) |
| Example 3-50 | R-50 | P-50(80) | — | PAG-1(10) | PGMEA(2250) | EL(2800) | DAA(550) |
| Example 3-51 | R-51 | P-1(80) | SQ-1(4) | — | PGMEA(2250) | EL(2800) | DAA(550) |
| Example 3-52 | R-52 | P-1(80) | AQ-1(4) | — | PGMEA(2250) | EL(2800) | DAA(550) |
| Example 3-53 | R-53 | P-1(80) | SQ-1(2) | — | PGMEA(2250) | EL(2800) | DAA(550) |
| | | | AQ-1(2) | | | | |

TABLE 6

| | Resist | Base polymer | Quencher | Acid generator | Solvent | Solvent 2 | Solvent 3 |
|---|---|---|---|---|---|---|---|
| | composition | (part by mass) | (part by mass) | (part by mass) | (part by mass) | (part by mass) | (part by mass) |
| Comparative | CR-1 | CP-1(80) | — | — | PGMEA(2250) | EL(2800) | DAA(550) |
| Example 3-1 | | | | | | | |
| Comparative | CR-2 | CP-2(80) | — | — | PGMEA(2250) | EL(2800) | DAA(550) |
| Example 3-2 | | | | | | | |
| Comparative | CR-3 | CP-3(80) | — | — | PGMEA(2250) | EL(2800) | DAA(550) |
| Example 3-3 | | | | | | | |
| Comparative | CR-4 | CP-4(80) | — | — | PGMEA(2250) | EL(2800) | DAA(550) |
| Example 3-4 | | | | | | | |
| Comparative | CR-5 | CP-5(80) | — | — | PGMEA(2250) | EL(2800) | DAA(550) |
| Example 3-5 | | | | | | | |
| Comparative | CR-6 | CP-6(80) | — | — | PGMEA(2250) | EL(2800) | DAA(550) |
| Example 3-6 | | | | | | | |
| Comparative | CR-7 | CP-7(80) | — | — | PGMEA(2250) | EL(2800) | DAA(550) |
| Example 3-7 | | | | | | | |
| Comparative | CR-8 | CP-8(80) | — | — | PGMEA(2250) | EL(2800) | DAA(550) |
| Example 3-8 | | | | | | | |
| Comparative | CR-9 | CP-9(80) | — | — | PGMEA(2250) | EL(2800) | DAA(550) |
| Example 3-9 | | | | | | | |
| Comparative | CR-10 | CP-10(80) | — | — | PGMEA(2250) | EL(2800) | DAA(550) |
| Example 3-10 | | | | | | | |
| Comparative | CR-11 | CP-11(80) | — | — | PGMEA(2250) | EL(2800) | DAA(550) |
| Example 3-11 | | | | | | | |
| Comparative | CR-12 | CP-12(80) | — | — | PGMEA(2250) | EL(2800) | DAA(550) |
| Example 3-12 | | | | | | | |
| Comparative | CR-13 | CP-13(80) | — | — | PGMEA(2250) | EL(2800) | DAA(550) |
| Example 3-13 | | | | | | | |
| Comparative | CR-14 | CP-14(80) | — | — | PGMEA(2250) | EL(2800) | DAA(550) |
| Example 3-14 | | | | | | | |
| Comparative | CR-15 | CP-15(80) | — | — | PGMEA(2250) | EL(2800) | DAA(550) |
| Example 3-15 | | | | | | | |
| Comparative | CR-16 | CP-16(80) | — | — | PGMEA(2250) | EL(2800) | DAA(550) |
| Example 3-16 | | | | | | | |
| Comparative | CR-17 | CP-17(80) | — | — | PGMEA(2250) | EL(2800) | DAA(550) |
| Example 3-17 | | | | | | | |
| Comparative | CR-18 | CP-18(80) | — | — | PGMEA(2250) | EL(2800) | DAA(550) |
| Example 3-18 | | | | | | | |
| Comparative | CR-19 | CP-19(80) | — | — | PGMEA(2250) | EL(2800) | DAA(550) |
| Example 3-19 | | | | | | | |
| Comparative | CR-20 | CP-20(80) | — | — | PGMEA(2250) | EL(2800) | DAA(550) |
| Example 3-20 | | | | | | | |
| Comparative | CR-21 | CP-21(80) | — | — | PGMEA(2250) | EL(2800) | DAA(550) |
| Example 3-21 | | | | | | | |
| Comparative | CR-22 | CP-22(80) | — | — | PGMEA(2250) | EL(2800) | DAA(550) |
| Example 3-22 | | | | | | | |
| Comparative | CR-23 | CP-23(80) | — | PAG-1(10) | PGMEA(2250) | EL(2800) | DAA(550) |
| Example 3-23 | | | | | | | |
| Comparative | CR-24 | CP-24(80) | — | — | PGMEA(2250) | EL(2800) | DAA(550) |
| Example 3-24 | | | | | | | |
| Comparative | CR-25 | CP-25(80) | — | — | PGMEA(2250) | EL(2800) | DAA(550) |
| Example 3-25 | | | | | | | |
| Comparative | CR-26 | CP-26(80) | — | — | PGMEA(2250) | EL(2800) | DAA(550) |
| Example 3-26 | | | | | | | |
| Comparative | CR-27 | CP-27(80) | — | — | PGMEA(2250) | EL(2800) | DAA(550) |
| Example 3-27 | | | | | | | |
| Comparative | CR-28 | CP-28(80) | — | — | PGMEA(2250) | EL(2800) | DAA(550) |
| Example 3-28 | | | | | | | |
| Comparative | CR-29 | CP-29(80) | — | — | PGMEA(2250) | EL(2800) | DAA(550) |
| Example 3-29 | | | | | | | |
| Comparative | CR-30 | CP-30(80) | — | — | PGMEA(2250) | EL(2800) | DAA(550) |
| Example 3-30 | | | | | | | |
| Comparative | CR-31 | CP-31(80) | — | — | PGMEA(2250) | EL(2800) | DAA(550) |
| Example 3-31 | | | | | | | |
| Comparative | CR-32 | CP-32(80) | — | PAG-2(10) | PGMEA(2250) | EL(2800) | DAA(550) |
| Example 3-32 | | | | | | | |
| Comparative | CR-33 | CP-33(80) | — | PAG-1(20) | PGMEA(2250) | EL(2800) | DAA(550) |
| Example 3-33 | | | | | | | |
| Comparative | CR-34 | CP-34(80) | — | PAG-1(20) | PGMEA(2250) | EL(2800) | DAA(550) |
| Example 3-34 | | | | | | | |
| Comparative | CR-35 | CP-35(80) | — | PAG-2(20) | PGMEA(2250) | EL(2800) | DAA(550) |
| Example 3-35 | | | | | | | |
| Comparative | CR-36 | CP-36(80) | — | PAG-1(20) | PGMEA(2250) | EL(2800) | DAA(550) |
| Example 3-36 | | | | | | | |
| Comparative | CR-37 | CP-37(80) | SQ-1(8) | — | PGMEA(2250) | EL(2800) | DAA(550) |
| Example 3-37 | | | | | | | |
| Comparative | CR-38 | CP-38(80) | SQ-1(8) | PAG-1(20) | PGMEA(2250) | EL(2800) | DAA(550) |
| Example 3-38 | | | | | | | |
| Comparative | CR-39 | CP-38(80) | AQ-1(8) | PAG-1(20) | PGMEA(2250) | EL(2800) | DAA(550) |
| Example 3-39 | | | | | | | |
| Comparative | CR-40 | CP-38(80) | SQ-1(4) | PAG-1(20) | PGMEA(2250) | EL(2800) | DAA(550) |
| Example 3-40 | | | AQ-1(4) | | | | |

The solvents, quenchers (SQ-1, AQ-1), and acid generators (PAG-1, PAG-2) in Tables 4 to 6 are as follows.
Solvents:
   PGMEA (propylene glycol monomethyl ether acetate)
   EL (ethyl lactate)
   DAA (diacetone alcohol)
Quenchers: SQ-1, AQ-1

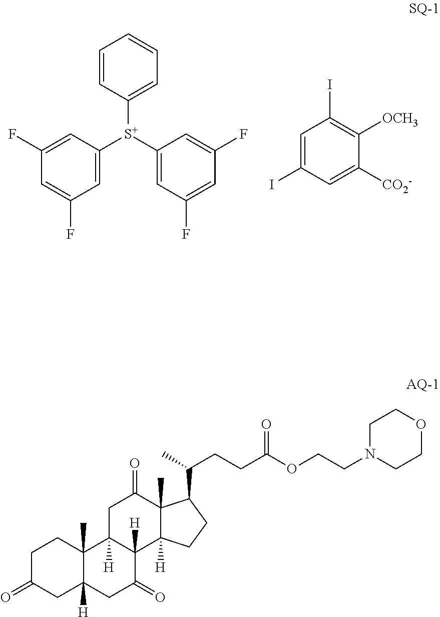

Acid generators: PAG-1, PAG-2

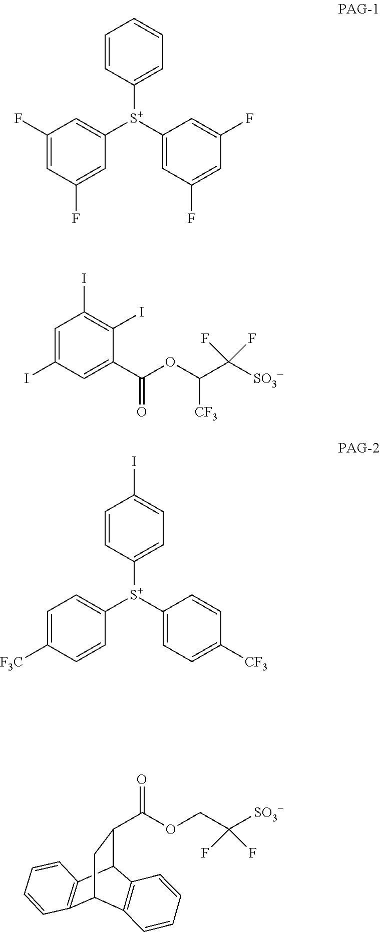

### [4]Evaluation for EUV Lithography (1)

### Examples 4-1 to 4-53, Comparative Examples 4-1 to 4-40

Onto a Si substrate on which the silicon-containing spin-on hard mask SHB-A940 (silicon content: 43% by mass) manufactured by Shin-Etsu Chemical Co., Ltd. with a film thickness of 20 nm had been formed, a chemically amplified resist composition (R-1 to R-53, CR-1 to CR-40) shown in Tables 4 to 6 was applied by spin coating, and prebaked by using a hot plate at 100° C. for 60 seconds to produce a resist film having a thickness of 50 nm. The resist film was exposed by using the EUV scanner NXE3300 (NA: 0.33, σ: 0.9/0.6, dipole illumination) manufactured by ASML with an LS pattern of 18 nm in on-wafer dimension and 36 nm in pitch while the dose and focus were varied (dose pitch: 1 mJ/cm², focus pitch: 0.020 μm), and after the exposure subjected to PEB at a temperature shown in Tables 7 to 9 for 60 seconds. Thereafter, puddle developing was performed with 2.38% by mass TMAH aqueous solution for 30 seconds, and the resultant was rinsed with a surfactant-containing rinsing material and spin-dried, giving a positive-type pattern.

The resulting LS pattern was observed with a critical dimension SEM (CG6300) manufactured by Hitachi High-Tech Corporation, and the sensitivity, EL, LWR, DOF, and collapse limit were evaluated with methods shown below. In addition, evaluation of post-develop defects was carried out for the resulting LS pattern. The results are shown in Tables 7 to 9.

### [Evaluation of Sensitivity]

An optimum dose, Eop (mJ/cm²), that gave an LS pattern of 18 nm in line width and 36 nm in pitch was determined as sensitivity. The smaller this value, the higher the sensitivity.

### [Evaluation of EL]

From a dose that allowed formation of the LS pattern within ±10% of the space width of 18 nm (16.2 to 19.8 nm), the EL (unit: %) was determined by using an expression shown below. The larger this value, the better the performance.

$$EL ( \% ) = ( \vert {E}_{1} - {E}_{2} \vert / Eop ) \times 100$$

E₁: optimum dose to give LS pattern of 16.2 nm in line width and 36 nm in pitch
E₂: optimum dose to give LS pattern of 19.8 nm in line width and 36 nm in pitch
Eop: optimum dose to give LS pattern of 18 nm in line width and 36 nm in pitch

### [Evaluation of LWR]

For the LS pattern obtained by irradiating at Eop, dimensions were measured at 10 points in the longitudinal direction of the line, the standard deviation (σ) was calculated from the results and multiplied by 3, and the resulting value (3σ) was determined as the LWR. As this value is smaller, a pattern with smaller roughness and more homogenous line width is given.

### [Evaluation of DOF]

For evaluation of depth of focus, a focus range that allowed formation of the LS pattern within ±10% of the dimension of 18 nm (16.2 to 19.8 nm) was determined. The larger this value, the wider the depth of focus.

### [Evaluation of Line Pattern Collapse Limit]

For the LS pattern, line dimensions were measured at 10 points in the longitudinal direction at different doses at the optimum focus. The smallest line dimension given without break-down was determined as the collapse limit dimension. The smaller this value, the more superior the collapse limit.

### [Evaluation of Post-Develop Defects]

For the LS pattern of 18 nm in line width and 36 nm in pitch that had been formed at the optimum dose, the defect inspection apparatus KLA2360 (product name) manufactured by KLA Corporation was applied to detect defects (defects/cm²) extracted from difference generated by pixel-by-pixel overlapping with a comparative image with the pixel size and threshold of the defect inspection apparatus set to 0.16 μm and 20, respectively, and the number of defects per unit area (defects/cm²) was calculated. Thereafter, all the defects were classified to extract post-develop defects therefrom through defect review, and the number of post-develop defects per unit area (defects/cm²) was calculated. A value of less than 0.5 was rated as A, a value of 0.5 or more and less than 1.0 as B, a value of 1.0 or more and less than 5.0 as C, and a value of 5.0 or more as D. A smaller value indicates better performance.

TABLE 7

| | | PEB | Optimum | | | | Collapse | Post- |
|---|---|---|---|---|---|---|---|---|
| | Resist | temperature | dose | EL | LWR | DOF | limit | develop |
| | composition | (° C.) | (mJ/cm²) | (%) | (nm) | (nm) | (nm) | defect |
| Example 4-1 | R-1 | 95 | 33 | 18 | 2.4 | 120 | 10.9 | A |
| Example 4-2 | R-2 | 100 | 33 | 19 | 2.3 | 110 | 10.9 | A |
| Example 4-3 | R-3 | 100 | 34 | 17 | 2.4 | 120 | 10.7 | A |
| Example 4-4 | R-4 | 95 | 33 | 18 | 2.5 | 110 | 11.2 | A |
| Example 4-5 | R-5 | 105 | 34 | 18 | 2.4 | 100 | 11.3 | A |
| Example 4-6 | R-6 | 100 | 33 | 17 | 2.5 | 120 | 10.9 | A |
| Example 4-7 | R-7 | 95 | 34 | 19 | 2.5 | 110 | 11.3 | A |
| Example 4-8 | R-8 | 95 | 34 | 17 | 2.4 | 100 | 11.2 | A |
| Example 4-9 | R-9 | 100 | 34 | 17 | 2.4 | 110 | 11.1 | A |
| Example 4-10 | R-10 | 100 | 34 | 18 | 2.5 | 120 | 11.1 | A |
| Example 4-11 | R-11 | 100 | 34 | 16 | 2.5 | 120 | 10.8 | A |
| Example 4-12 | R-12 | 95 | 35 | 18 | 2.5 | 110 | 11.1 | A |
| Example 4-13 | R-13 | 105 | 35 | 17 | 2.4 | 120 | 11.2 | A |
| Example 4-14 | R-14 | 100 | 33 | 18 | 2.4 | 100 | 11.2 | A |
| Example 4-15 | R-15 | 95 | 35 | 17 | 2.3 | 110 | 11.0 | A |
| Example 4-16 | R-16 | 95 | 33 | 19 | 2.4 | 120 | 10.9 | A |
| Example 4-17 | R-17 | 100 | 33 | 17 | 2.5 | 100 | 10.9 | A |
| Example 4-18 | R-18 | 95 | 33 | 16 | 2.4 | 110 | 11.1 | A |
| Example 4-19 | R-19 | 95 | 35 | 18 | 2.5 | 120 | 11.3 | A |
| Example 4-20 | R-20 | 100 | 33 | 17 | 2.4 | 110 | 11.1 | A |
| Example 4-21 | R-21 | 100 | 34 | 17 | 2.4 | 110 | 11.2 | A |
| Example 4-22 | R-22 | 95 | 34 | 16 | 2.5 | 110 | 11.1 | A |
| Example 4-23 | R-23 | 100 | 33 | 18 | 2.6 | 100 | 11.4 | A |
| Example 4-24 | R-24 | 95 | 34 | 17 | 2.4 | 110 | 11.4 | A |
| Example 4-25 | R-25 | 100 | 33 | 19 | 2.3 | 120 | 11.5 | A |
| Example 4-26 | R-26 | 100 | 33 | 17 | 2.3 | 110 | 10.8 | A |
| Example 4-27 | R-27 | 95 | 33 | 19 | 2.4 | 110 | 11.1 | A |
| Example 4-28 | R-28 | 100 | 33 | 18 | 2.5 | 110 | 11.2 | A |
| Example 4-29 | R-29 | 100 | 34 | 16 | 2.4 | 110 | 11.3 | A |
| Example 4-30 | R-30 | 95 | 34 | 17 | 2.4 | 110 | 11.4 | A |

TABLE 8

| | | PEB | Optimum | | | | Collapse | Post- |
|---|---|---|---|---|---|---|---|---|
| | Resist | temperature | dose | EL | LWR | DOF | limit | develop |
| | composition | (° C.) | (mJ/cm²) | (%) | (nm) | (nm) | (nm) | defect |
| Example 4-31 | R-31 | 100 | 34 | 19 | 2.5 | 110 | 11.1 | A |
| Example 4-32 | R-32 | 100 | 34 | 17 | 2.5 | 100 | 11.3 | A |
| Example 4-33 | R-33 | 100 | 33 | 18 | 2.3 | 110 | 10.9 | A |
| Example 4-34 | R-34 | 95 | 34 | 18 | 2.4 | 120 | 11.1 | A |
| Example 4-35 | R-35 | 100 | 34 | 17 | 2.4 | 110 | 11.3 | A |
| Example 4-36 | R-36 | 95 | 33 | 19 | 2.5 | 110 | 11.2 | A |
| Example 4-37 | R-37 | 100 | 33 | 18 | 2.4 | 120 | 11.2 | A |
| Example 4-38 | R-38 | 100 | 34 | 18 | 2.4 | 110 | 11.1 | A |
| Example 4-39 | R-39 | 95 | 35 | 17 | 2.4 | 100 | 11.1 | A |
| Example 4-40 | R-40 | 100 | 34 | 16 | 2.5 | 110 | 11.4 | A |
| Example 4-41 | R-41 | 95 | 33 | 18 | 2.6 | 110 | 11.2 | A |
| Example 4-42 | R-42 | 95 | 35 | 17 | 2.4 | 120 | 11.1 | A |
| Example 4-43 | R-43 | 100 | 34 | 19 | 2.4 | 110 | 10.9 | A |
| Example 4-44 | R-44 | 100 | 34 | 17 | 2.3 | 100 | 11.0 | A |
| Example 4-45 | R-45 | 95 | 35 | 18 | 2.5 | 120 | 11.3 | A |
| Example 4-46 | R-46 | 95 | 33 | 17 | 2.6 | 110 | 11.2 | A |
| Example 4-47 | R-47 | 100 | 34 | 17 | 2.6 | 110 | 10.8 | A |
| Example 4-48 | R-48 | 95 | 35 | 18 | 2.5 | 120 | 11.2 | A |
| Example 4-49 | R-49 | 95 | 33 | 18 | 2.4 | 110 | 10.7 | A |
| Example 4-50 | R-50 | 100 | 33 | 18 | 2.3 | 110 | 11.2 | A |
| Example 4-51 | R-51 | 95 | 34 | 17 | 2.3 | 120 | 11.1 | A |
| Example 4-52 | R-52 | 95 | 34 | 16 | 2.4 | 110 | 11.3 | A |
| Example 4-53 | R-53 | 100 | 33 | 18 | 2.3 | 110 | 11.1 | A |

TABLE 9

| | | PEB | Optimum | | | | Collapse | |
|---|---|---|---|---|---|---|---|---|
| | Resist | temperature | dose | EL | LWR | DOF | limit | Post-devel |
| | composition | (° C.) | (mJ/cm²) | (%) | (nm) | (nm) | (nm) | op defect |
| Comparative Example 4-1 | CR-1 | 100 | 38 | 13 | 3 | 90 | 12.1 | B |
| Comparative Example 4-2 | CR-2 | 95 | 39 | 12 | 3.1 | 90 | 12.7 | C |
| Comparative Example 4-3 | CR-3 | 100 | 37 | 14 | 3.2 | 80 | 12.2 | B |
| Comparative Example 4-4 | CR-4 | 105 | 38 | 14 | 3.1 | 90 | 12.4 | C |
| Comparative Example 4-5 | CR-5 | 100 | 38 | 14 | 3 | 80 | 12.6 | B |
| Comparative Example 4-6 | CR-6 | 95 | 41 | 12 | 2.9 | 90 | 12.4 | C |
| Comparative Example 4-7 | CR-7 | 100 | 37 | 12 | 3.2 | 90 | 13.2 | C |
| Comparative Example 4-8 | CR-8 | 105 | 39 | 14 | 3.1 | 90 | 12.6 | B |
| Comparative Example 4-9 | CR-9 | 95 | 41 | 14 | 3.1 | 80 | 13.2 | B |
| Comparative Example 4-10 | CR-10 | 100 | 38 | 12 | 3 | 90 | 13.3 | C |
| Comparative Example 4-11 | CR-11 | 95 | 38 | 13 | 3.1 | 90 | 12.7 | B |
| Comparative Example 4-12 | CR-12 | 100 | 37 | 14 | 3.2 | 90 | 12.6 | C |
| Comparative Example 4-13 | CR-13 | 100 | 42 | 12 | 3.2 | 90 | 13.1 | C |
| Comparative Example 4-14 | CR-14 | 95 | 38 | 13 | 3.1 | 90 | 12.6 | C |
| Comparative Example 4-15 | CR-15 | 105 | 41 | 12 | 3.1 | 90 | 12.8 | B |
| Comparative Example 4-16 | CR-16 | 100 | 38 | 12 | 3 | 90 | 12.4 | C |
| Comparative Example 4-17 | CR-17 | 100 | 38 | 11 | 3.1 | 80 | 13.1 | B |
| Comparative Example 4-18 | CR-18 | 95 | 39 | 13 | 2.9 | 80 | 13.1 | C |
| Comparative Example 4-19 | CR-19 | 100 | 38 | 12 | 3.1 | 90 | 12.5 | C |
| Comparative Example 4-20 | CR-20 | 95 | 39 | 14 | 3 | 80 | 13.1 | B |
| Comparative Example 4-21 | CR-21 | 100 | 42 | 13 | 2.9 | 90 | 12.4 | C |
| Comparative Example 4-22 | CR-22 | 100 | 38 | 15 | 3 | 90 | 13.1 | C |
| Comparative Example 4-23 | CR-23 | 100 | 38 | 13 | 3.2 | 80 | 12.7 | B |
| Comparative Example 4-24 | CR-24 | 95 | 41 | 15 | 3.3 | 90 | 12.6 | B |
| Comparative Example 4-25 | CR-25 | 100 | 37 | 12 | 3.1 | 90 | 12.6 | C |
| Comparative Example 4-26 | CR-26 | 105 | 38 | 14 | 3.2 | 80 | 13.1 | B |
| Comparative Example 4-27 | CR-27 | 100 | 38 | 13 | 3.1 | 90 | 12.4 | C |
| Comparative Example 4-28 | CR-28 | 95 | 37 | 15 | 3.3 | 90 | 12.6 | C |
| Comparative Example 4-29 | CR-29 | 100 | 41 | 14 | 3.2 | 90 | 12.3 | B |
| Comparative Example 4-30 | CR-30 | 95 | 37 | 13 | 3.1 | 90 | 12.6 | C |
| Comparative Example 4-31 | CR-31 | 100 | 38 | 12 | 3 | 90 | 12.4 | C |
| Comparative Example 4-32 | CR-32 | 95 | 40 | 13 | 3.3 | 80 | 13.1 | B |
| Comparative Example 4-33 | CR-33 | 95 | 39 | 14 | 3.5 | 90 | 12.9 | C |
| Comparative Example 4-34 | CR-34 | 100 | 39 | 13 | 3.2 | 80 | 13.1 | C |
| Comparative Example 4-35 | CR-35 | 100 | 38 | 15 | 3.4 | 90 | 12.9 | B |
| Comparative Example 4-36 | CR-36 | 105 | 38 | 13 | 3.5 | 70 | 13.1 | C |
| Comparative Example 4-37 | CR-37 | 95 | 37 | 14 | 3.6 | 70 | 13.3 | C |
| Comparative Example 4-38 | CR-38 | 100 | 40 | 13 | 3.3 | 70 | 13.2 | B |
| Comparative Example 4-39 | CR-39 | 95 | 38 | 13 | 3.5 | 70 | 13.3 | C |
| Comparative Example 4-40 | CR-40 | 100 | 37 | 14 | 3.6 | 70 | 14.1 | C |

It was found from the results shown in Tables 7 to 9 that the chemically amplified resist compositions each obtained with a base polymer containing a repeating unit derived from the onium salt monomer of the present invention were superior in EL, LWR, and DOF with good sensitivity. In addition, they exhibited small collapse limit values, and thus were confirmed to be resistant to pattern collapse even in fine patterning. They were further confirmed to have reduced post-develop defects. Accordingly, the chemically amplified resist composition of the present invention was demonstrated to be suitable as a material for EUV lithography.

### [5]Evaluation for EUV Lithography (2)

### Examples 5-1 to 5-53, Comparative Examples 5-1 to 5-40

Onto a Si substrate on which the silicon-containing spin-on hard mask SHB-A940 (silicon content: 43% by mass) manufactured by Shin-Etsu Chemical Co., Ltd. with a film thickness of 20 nm had been formed, a chemically amplified resist composition (R-1 to R-53, CR-1 to CR-40) shown in Tables 4 to 6 was applied by spin coating, and prebaked by using a hot plate at 105° C. for 60 seconds to produce a resist film having a thickness of 50 nm. The resist film was exposed by using the EUV scanner NXE3400 (NA: 0.33, o: 0.9/0.6, quadrupole illumination, mask with hole pattern having on-wafer dimension of 46 nm in pitch, +20% bias) manufactured by ASML, PEB was performed by using a hot plate at a temperature shown in Tables 10 to 12 for 60 seconds, and developing was performed with 2.38% by mass TMAH aqueous solution for 30 seconds to form a hole pattern having a dimension of 23 nm.

A dose that allowed formation with a hole dimension of 23 nm was measured as the sensitivity by using a critical dimension SEM (CG6300) manufactured by Hitachi High-Tech Corporation, and the dimensions of 50 holes at that time were measured, the standard deviation (σ) was calculated from the results and multiplied by 3, and the resulting value (3σ) was determined as the critical dimension uniformity (CDU). The results are shown in Tables 10 to 12.

TABLE 10

| | | PEB | Optimum | |
|---|---|---|---|---|
| | Resist | temperature | dose | CDU |
| | composition | (° C.) | (mJ/cm²) | (nm) |
| Example 5-1 | R-1 | 95 | 23 | 2.2 |
| Example 5-2 | R-2 | 95 | 24 | 2.4 |
| Example 5-3 | R-3 | 90 | 22 | 2.3 |
| Example 5-4 | R-4 | 90 | 23 | 2.4 |
| Example 5-5 | R-5 | 90 | 22 | 2.4 |
| Example 5-6 | R-6 | 95 | 23 | 2.3 |
| Example 5-7 | R-7 | 95 | 24 | 2.2 |
| Example 5-8 | R-8 | 90 | 23 | 2.5 |
| Example 5-9 | R-9 | 95 | 24 | 2.5 |
| Example 5-10 | R-10 | 95 | 23 | 2.6 |
| Example 5-11 | R-11 | 95 | 23 | 2.3 |
| Example 5-12 | R-12 | 90 | 23 | 2.4 |
| Example 5-13 | R-13 | 90 | 24 | 2.3 |
| Example 5-14 | R-14 | 90 | 25 | 2.4 |
| Example 5-15 | R-15 | 90 | 24 | 2.4 |
| Example 5-16 | R-16 | 85 | 23 | 2.4 |
| Example 5-17 | R-17 | 95 | 22 | 2.5 |
| Example 5-18 | R-18 | 95 | 24 | 2.3 |
| Example 5-19 | R-19 | 90 | 23 | 2.4 |
| Example 5-20 | R-20 | 95 | 23 | 2.5 |
| Example 5-21 | R-21 | 95 | 23 | 2.4 |
| Example 5-22 | R-22 | 95 | 24 | 2.3 |
| Example 5-23 | R-23 | 95 | 25 | 2.3 |
| Example 5-24 | R-24 | 95 | 23 | 2.2 |
| Example 5-25 | R-25 | 95 | 23 | 2.4 |
| Example 5-26 | R-26 | 95 | 24 | 2.3 |
| Example 5-27 | R-27 | 95 | 24 | 2.3 |
| Example 5-28 | R-28 | 95 | 23 | 2.5 |
| Example 5-29 | R-29 | 90 | 24 | 2.3 |
| Example 5-30 | R-30 | 95 | 23 | 2.3 |

TABLE 11

| | | PEB | Optimum | |
|---|---|---|---|---|
| | Resist | temperature | dose | CDU |
| | composition | (° C.) | (mJ/cm²) | (nm) |
| Example 5-31 | R-31 | 95 | 23 | 2.3 |
| Example 5-32 | R-32 | 95 | 23 | 2.3 |
| Example 5-33 | R-33 | 95 | 22 | 2.2 |
| Example 5-34 | R-34 | 90 | 23 | 2.3 |
| Example 5-35 | R-35 | 95 | 24 | 2.4 |
| Example 5-36 | R-36 | 95 | 23 | 2.4 |
| Example 5-37 | R-37 | 90 | 23 | 2.3 |
| Example 5-38 | R-38 | 90 | 24 | 2.3 |
| Example 5-39 | R-39 | 95 | 25 | 2.5 |
| Example 5-40 | R-40 | 95 | 22 | 2.2 |
| Example 5-41 | R-41 | 95 | 23 | 2.4 |
| Example 5-42 | R-42 | 90 | 24 | 2.5 |
| Example 5-43 | R-43 | 95 | 23 | 2.4 |
| Example 5-44 | R-44 | 95 | 23 | 2.5 |
| Example 5-45 | R-45 | 90 | 25 | 2.5 |
| Example 5-46 | R-46 | 95 | 24 | 2.4 |
| Example 5-47 | R-47 | 90 | 25 | 2.3 |
| Example 5-48 | R-48 | 90 | 24 | 2.5 |
| Example 5-49 | R-49 | 95 | 25 | 2.4 |
| Example 5-50 | R-50 | 90 | 24 | 2.5 |
| Example 5-51 | R-51 | 95 | 24 | 2.3 |
| Example 5-52 | R-52 | 95 | 23 | 2.4 |
| Example 5-53 | R-53 | 95 | 24 | 2.5 |

TABLE 12

| | | PEB | Optimum | |
|---|---|---|---|---|
| | Resist | temperature | dose | CDU |
| | composition | (° C.) | (mJ/cm²) | (nm) |
| Comparative | CR-1 | 95 | 29 | 2.9 |
| Example 5-1 | | | | |
| Comparative | CR-2 | 90 | 28 | 2.8 |
| Example 5-2 | | | | |
| Comparative | CR-3 | 90 | 28 | 2.9 |
| Example 5-3 | | | | |
| Comparative | CR-4 | 95 | 28 | 2.8 |
| Example 5-4 | | | | |
| Comparative | CR-5 | 90 | 29 | 2.9 |
| Example 5-5 | | | | |
| Comparative | CR-6 | 95 | 28 | 2.7 |
| Example 5-6 | | | | |
| Comparative | CR-7 | 95 | 27 | 2.8 |
| Example 5-7 | | | | |
| Comparative | CR-8 | 95 | 28 | 2.8 |
| Example 5-8 | | | | |
| Comparative | CR-9 | 90 | 28 | 2.9 |
| Example 5-9 | | | | |
| Comparative | CR-10 | 95 | 27 | 2.7 |
| Example 5-10 | | | | |
| Comparative | CR-11 | 95 | 29 | 2.9 |
| Example 5-11 | | | | |
| Comparative | CR-12 | 90 | 29 | 2.8 |
| Example 5-12 | | | | |
| Comparative | CR-13 | 90 | 28 | 2.8 |
| Example 5-13 | | | | |
| Comparative | CR-14 | 90 | 29 | 2.9 |
| Example 5-14 | | | | |
| Comparative | CR-15 | 95 | 29 | 3 |
| Example 5-15 | | | | |
| Comparative | CR-16 | 85 | 30 | 2.8 |
| Example 5-16 | | | | |
| Comparative | CR-17 | 95 | 28 | 2.8 |
| Example 5-17 | | | | |
| Comparative | CR-18 | 95 | 27 | 2.9 |
| Example 5-18 | | | | |
| Comparative | CR-19 | 90 | 29 | 2.8 |
| Example 5-19 | | | | |
| Comparative | CR-20 | 95 | 27 | 2.9 |
| Example 5-20 | | | | |
| Comparative | CR-21 | 95 | 28 | 2.7 |
| Example 5-21 | | | | |
| Comparative | CR-22 | 95 | 29 | 2.8 |
| Example 5-22 | | | | |
| Comparative | CR-23 | 95 | 28 | 2.9 |
| Example 5-23 | | | | |
| Comparative | CR-24 | 95 | 28 | 2.9 |
| Example 5-24 | | | | |
| Comparative | CR-25 | 95 | 28 | 2.8 |
| Example 5-25 | | | | |
| Comparative | CR-26 | 95 | 29 | 2.9 |
| Example 5-26 | | | | |
| Comparative | CR-27 | 95 | 27 | 2.8 |
| Example 5-27 | | | | |
| Comparative | CR-28 | 95 | 29 | 2.7 |
| Example 5-28 | | | | |
| Comparative | CR-29 | 90 | 29 | 2.8 |
| Example 5-29 | | | | |
| Comparative | CR-30 | 90 | 28 | 2.9 |
| Example 5-30 | | | | |
| Comparative | CR-31 | 95 | 27 | 2.9 |
| Example 5-31 | | | | |
| Comparative | CR-32 | 90 | 30 | 2.8 |
| Example 5-32 | | | | |
| Comparative | CR-33 | 95 | 28 | 2.9 |
| Example 5-33 | | | | |
| Comparative | CR-34 | 95 | 29 | 2.9 |
| Example 5-34 | | | | |
| Comparative | CR-35 | 95 | 29 | 2.9 |
| Example 5-35 | | | | |
| Comparative | CR-36 | 95 | 28 | 2.9 |
| Example 5-36 | | | | |
| Comparative | CR-37 | 90 | 30 | 3.1 |
| Example 5-37 | | | | |
| Comparative | CR-38 | 90 | 30 | 3.2 |
| Example 5-38 | | | | |
| Comparative | CR-39 | 90 | 31 | 3.1 |
| Example 5-39 | | | | |
| Comparative | CR-40 | 90 | 31 | 3.1 |
| Example 5-40 | | | | |

From the results shown in Tables 10 to 12, the chemically amplified resist composition of the present invention was confirmed to have good sensitivity and be superior in CDU.

### [6]Evaluation of Dry Etching Resistance

### Examples 6-1 to 6-50, Comparative Examples 6-1 to 6-38

In 10 g of cyclohexanone, 2 g of a polymer shown in Tables 1 to 3 polymer (polymers P-1 to P-50, comparative polymers CP-1 to CP-38) was dissolved, the resultant was filtered through a filter of 0.2 μm in size, the resulting polymer solution was applied onto a Si substrate by spin coating to form a film with a thickness of 300 nm, and the film was evaluated under the following conditions. Etching test with CHF₃/CF₄-based gas:

The difference between the thicknesses of the polymer film before and after etching was determined by using the dry etching apparatus TE-8500P manufactured by Tokyo Electron Ltd.

The etching conditions were as follows.
Chamber pressure: 40 Pa
RF power: 1000 W
Gap: 9 mm
CHF₃ gas flow rate: 30 mL/min
CF₄ gas flow rate: 30 mL/min
Ar gas flow rate: 100 mL/min
Time: 60 sec

According to this evaluation, a smaller difference in film thickness, that is, a smaller reduction indicates higher etching resistance.

Tables 13 to 15 show the results for dry etching resistance.

TABLE 13

| | | CHF₃/CF₄-based gas |
|---|---|---|
| | Polymer | etching rate (nm/min) |
| Example 6-1 | P-1 | 94 |
| Example 6-2 | P-2 | 95 |
| Example 6-3 | P-3 | 97 |
| Example 6-4 | P-4 | 94 |
| Example 6-5 | P-5 | 95 |
| Example 6-6 | P-6 | 94 |
| Example 6-7 | P-7 | 95 |
| Example 6-8 | P-8 | 94 |
| Example 6-9 | P-9 | 95 |
| Example 6-10 | P-10 | 94 |
| Example 6-11 | P-11 | 96 |
| Example 6-12 | P-12 | 96 |
| Example 6-13 | P-13 | 94 |
| Example 6-14 | P-14 | 96 |
| Example 6-15 | P-15 | 95 |
| Example 6-16 | P-16 | 95 |
| Example 6-17 | P-17 | 95 |
| Example 6-18 | P-18 | 94 |
| Example 6-19 | P-19 | 96 |
| Example 6-20 | P-20 | 95 |
| Example 6-21 | P-21 | 94 |
| Example 6-22 | P-22 | 94 |
| Example 6-23 | P-23 | 96 |
| Example 6-24 | P-24 | 95 |
| Example 6-25 | P-25 | 96 |
| Example 6-26 | P-26 | 95 |
| Example 6-27 | P-27 | 95 |
| Example 6-28 | P-28 | 95 |
| Example 6-29 | P-29 | 95 |
| Example 6-30 | P-30 | 96 |

TABLE 14

| | | CHF₃/CF₄-based gas |
|---|---|---|
| | Polymer | etching rate (nm/min) |
| Example 6-31 | P-31 | 95 |
| Example 6-32 | P-32 | 94 |
| Example 6-33 | P-33 | 95 |
| Example 6-34 | P-34 | 95 |
| Example 6-35 | P-35 | 95 |
| Example 6-36 | P-36 | 94 |
| Example 6-37 | P-37 | 94 |
| Example 6-38 | P-38 | 95 |
| Example 6-39 | P-39 | 96 |
| Example 6-40 | P-40 | 96 |
| Example 6-41 | P-41 | 96 |
| Example 6-42 | P-42 | 95 |
| Example 6-43 | P-43 | 94 |
| Example 6-44 | P-44 | 95 |
| Example 6-45 | P-45 | 95 |
| Example 6-46 | P-46 | 96 |
| Example 6-47 | P-47 | 96 |
| Example 6-48 | P-48 | 95 |
| Example 6-49 | P-49 | 95 |
| Example 6-50 | P-50 | 94 |

TABLE 15

| | | CHF₃/CF₄-based gas |
|---|---|---|
| | Polymer | etching rate (nm/min) |
| Comparative | CP-1 | 101 |
| Example 6-1 | | |
| Comparative | CP-2 | 102 |
| Example 6-2 | | |
| Comparative | CP-3 | 102 |
| Example 6-3 | | |
| Comparative | CP-4 | 104 |
| Example 6-4 | | |
| Comparative | CP-5 | 103 |
| Example 6-5 | | |
| Comparative | CP-6 | 102 |
| Example 6-6 | | |
| Comparative | CP-7 | 105 |
| Example 6-7 | | |
| Comparative | CP-8 | 103 |
| Example 6-8 | | |
| Comparative | CP-9 | 104 |
| Example 6-9 | | |
| Comparative | CP-10 | 101 |
| Example 6-10 | | |
| Comparative | CP-11 | 105 |
| Example 6-11 | | |
| Comparative | CP-12 | 103 |
| Example 6-12 | | |
| Comparative | CP-13 | 104 |
| Example 6-13 | | |
| Comparative | CP-14 | 106 |
| Example 6-14 | | |
| Comparative | CP-15 | 105 |
| Example 6-15 | | |
| Comparative | CP-16 | 106 |
| Example 6-16 | | |
| Comparative | CP-17 | 105 |
| Example 6-17 | | |
| Comparative | CP-18 | 106 |
| Example 6-18 | | |
| Comparative | CP-19 | 103 |
| Example 6-19 | | |
| Comparative | CP-20 | 102 |
| Example 6-20 | | |
| Comparative | CP-21 | 105 |
| Example 6-21 | | |
| Comparative | CP-22 | 102 |
| Example 6-22 | | |
| Comparative | CP-23 | 107 |
| Example 6-23 | | |
| Comparative | CP-24 | 105 |
| Example 6-24 | | |
| Comparative | CP-25 | 101 |
| Example 6-25 | | |
| Comparative | CP-26 | 104 |
| Example 6-26 | | |
| Comparative | CP-27 | 103 |
| Example 6-27 | | |
| Comparative | CP-28 | 108 |
| Example 6-28 | | |
| Comparative | CP-29 | 103 |
| Example 6-29 | | |
| Comparative | CP-30 | 105 |
| Example 6-30 | | |
| Comparative | CP-31 | 107 |
| Example 6-31 | | |
| Comparative | CP-32 | 106 |
| Example 6-32 | | |
| Comparative | CP-33 | 104 |
| Example 6-33 | | |
| Comparative | CP-34 | 106 |
| Example 6-34 | | |
| Comparative | CP-35 | 104 |
| Example 6-35 | | |
| Comparative | CP-36 | 106 |
| Example 6-36 | | |
| Comparative | CP-37 | 107 |
| Example 6-37 | | |
| Comparative | CP-38 | 113 |
| Example 6-38 | | |

From the results shown in Tables 13 to 15, the polymer of the present invention was confirmed to have superior dry etching resistance to CHF₃/CF₄-based gas.

The present description includes the following embodiments.

[1]A sulfonium salt monomer represented by the following general formula (a):

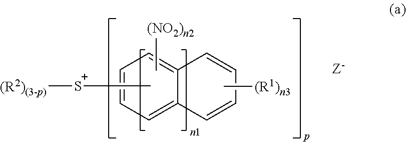

wherein p is 1, 2, or 3, n1 is 0 or 1, n2 is 1 or 2, and n3 is an integer of 0 to 6, provided that 1≤n2+n3≤5 is satisfied if n1 is 0, and 1≤n2+n3≤7 is satisfied if n1 is 1;
R¹ is a halogen atom, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein if n3 is 2 to 6, R¹ groups are identical to or different from each other, and any two R¹ groups are optionally bound together to form a ring together with the carbon atoms to which the two groups are bound;
R² is a halogen atom or a hydrocarbyl group having 1 to 30 carbon atoms and optionally containing a heteroatom, wherein if p is 1, the two R² groups are identical to or different from each other, and any two of the three substituents bound to S⁺ are optionally bound together to form a ring together with the sulfur atom to which the two substituents are bound; and
Z⁻ is a carboxylate anion having an aromatic vinyl structure and an iodine atom.

[2] The sulfonium salt monomer according to [1], wherein the sulfonium salt monomer is represented by the following general formula (a1):

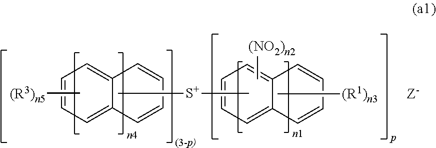

wherein p, n1 to n3, R¹, and Z⁻ are as specified above;
n4 is 0 or 1, and n5 is an integer of 0 to 5; and
R³ is a halogen atom, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein if n5 is 2 to 5, R³ groups are identical to or different from each other, and any two R³ groups are optionally bound together to form a ring together with the carbon atoms to which the two groups are bound.

[3] The sulfonium salt monomer according to according to [1] or [2], wherein the Z⁻ is an anion represented by the following general formula (Z):

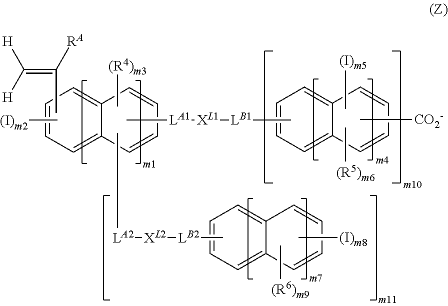

wherein m1 is 0 or 1, m2 is an integer of 0 to 4, m3 is an integer of 0 to 3, m4 is 0 or 1, m5 is an integer of 0 to 4, m6 is an integer of 0 to 3, m7 is 0 or 1, m8 is an integer of 0 to 4, m9 is an integer of 0 to 3, m10 is 0 or 1, and m11 is 0 or 1, provided that 0≤m2+m3+m11≤4 is satisfied if m1 is 0, and 0≤m2+m3+m11≤6 is satisfied if m1 is 1, that 0≤m5+m6≤4 is satisfied if m4 is 0, and 0≤m5+m6≤6 is satisfied if m4 is 1, that 0≤m8+m9≤5 is satisfied if m7 is 0, and 0≤m8+m9≤7 is satisfied if m7 is 1, and that 1≤m2+m5+m8≤4 is satisfied;
R^{A} is a hydrogen atom, a fluorine atom, a methyl group, or a trifluoromethyl group;
R⁴, R⁵, and R⁶ are each a halogen atom being not an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein: if m3 is 2 or 3, R⁴ groups are identical to or different from each other, and any two R⁴ groups are optionally bound together to form a ring together with the carbon atoms to which the two groups are bound; if m6 is 2 or 3, R⁵ groups are identical to or different from each other, and any two R⁵ groups are optionally bound together to form a ring together with the carbon atoms to which the two groups are bound; and if m9 is 2 or 3, R⁶ groups are identical to or different from each other, and any two R⁶ groups are optionally bound together to form a ring together with the carbon atoms to which the two groups are bound;
L^{A1}, L^{A2}, L^{B1}, and L^{B2} are each independently a single bond, an ether bond, a carbonyl group, an ester bond, a sulfonate bond, an amide bond, a sulfonamide bond, a carbonate bond, or a carbamate bond; and
X^{L1} and X^{L2} are each independently a single bond or a hydrocarbylene group having 1 to 40 carbon atoms and optionally containing a heteroatom.

[4]A sulfonium salt quencher comprising the sulfonium salt monomer according to any one of [1] to [3].

[5]A polymer comprising a repeating unit derived from the sulfonium salt quencher according to [4].

[6] The polymer according to [5], wherein the polymer further comprises a repeating unit or repeating units represented by either one or both of the following general formulae (b1) and (b2):

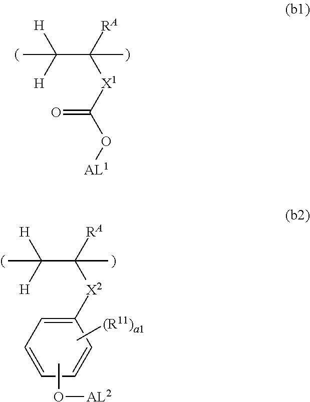

wherein R^{A} groups are each independently a hydrogen atom, a fluorine atom, a methyl group, or a trifluoromethyl group;
X¹ is a single bond, a phenylene group, a naphthylene group, *—C(═O)—O—X¹¹—, or *—C(═O)—NH—X¹¹—, wherein the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring;
X² is a single bond, *—C(═O)—O—, or *—C(═O)—NH—;
* indicates bonding to a carbon atom of a main chain;
R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom, wherein if a1 is 2 or more, R¹¹ groups are identical to or different from each other;
AL¹ and AL² are each independently an acid-unstable group; and
a1 is an integer of 0 to 4.

[7] The polymer according to [5] or [6], wherein the polymer further comprises a repeating unit represented by the following general formula (b3):

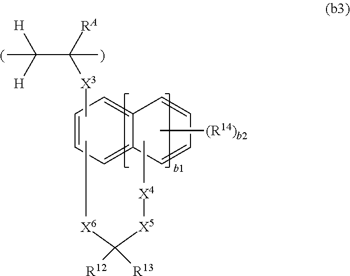

wherein b1 is 0 or 1, and b2 is an integer of 0 to 3 if b1 is 0, and an integer of 0 to 5 if b1 is 1;
R^{A} is a hydrogen atom, a fluorine atom, a methyl group, or a trifluoromethyl group;
X³ is a single bond, *—C(═O)—O—, or *—C(═O)—NH—, wherein * indicates bonding to a carbon atom of a main chain;
R¹² and R¹³ are each independently a hydrogen atom or a hydrocarbyl group having 1 to 20 carbon atoms and optionally containing a heteroatom, wherein R¹² and R¹³ are optionally bound together to form a ring together with the carbon atom to which the groups are bound;
R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A}) (R^{14B}), wherein R^{14A} and R^{14B} are each independently a hydrogen atom or a hydrocarbyl group having 1 to 6 carbon atoms, and if b2 is 2 or more, R¹⁴ groups are identical to or different from each other, and any multiple R¹⁴ groups are optionally bound together to form a ring together with the carbon atoms of the aromatic ring or aromatic rings to which the groups are bound;
X⁴ is a single bond, an aliphatic hydrocarbylene group having 1 to 4 carbon atoms, a carbonyl group, a sulfonyl group, or a group given by combining any of these; and
X⁵ and X⁶ are each independently an oxygen atom or a sulfur atom, provided that X⁴ and X⁶ are bound to adjacent carbon atoms in the aromatic ring or aromatic rings.

[8] The polymer according to any one of [5] to [7], wherein the polymer further comprises a repeating unit represented by the following general formula (c):

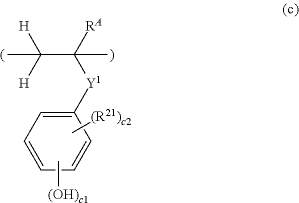

wherein R^{A} is a hydrogen atom, a fluorine atom, a methyl group, or a trifluoromethyl group;
Y¹ is a single bond, *—C(═O)—O—, or *—C(═O)—NH—, wherein * indicates bonding to a carbon atom of a main chain;
R²¹ is a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
c1 is an integer of 1 to 4, and c2 is an integer of 0 to 3, provided that 1≤c1+c2≤5 is satisfied.

[9] The polymer according to according to any one of [5] to [8], wherein the polymer further comprises a repeating unit derived from an onium salt monomer containing a fluorosulfonate anion having a polymerizable group and at least one iodine atom and a sulfonium cation.

[10] The polymer according to any one of [5] to [9], wherein the polymer further comprises a repeating unit represented by the following general formula (e):

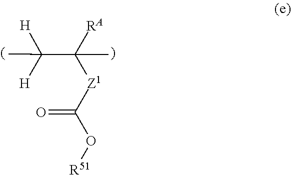

wherein R^{A} is a hydrogen atom, a fluorine atom, a methyl group, or a trifluoromethyl group;
Z¹ is a single bond, a phenylene group, a naphthylene group, *—C(═O)—O—Z¹¹—, or *—C(═O)—NH—Z¹¹—, wherein the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, * indicates bonding to a carbon atom of a main chain, Z¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring; and
R⁵¹ is a hydrogen atom or a group having 1 to 20 carbon atoms and containing at least one structure selected from a hydroxy group being not a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—).

[11]A chemically amplified resist composition comprising (A) a base polymer containing the polymer according to any one of [5] to [10]

[12] The chemically amplified resist composition according to [11], wherein the chemically amplified resist composition further comprises one or more selected from (B) an organic solvent, (C) a quencher other than the sulfonium salt quencher, (D) an acid generator, and (E) a surfactant.

[13]A patterning method comprising: a step of forming a resist film on a substrate by using the chemically amplified resist composition according to [11] or [12]; a step of exposing the resist film to a high-energy ray; and a step of developing the exposed resist film by using a developer.

[14] The patterning method according to [13], wherein ArF excimer laser light having a wavelength of 193 nm, KrF excimer laser light having a wavelength of 248 nm, an electron beam, or an extreme ultraviolet ray having a wavelength of 3 to 15 nm is used as the high-energy ray.

The present invention is not limited to the above embodiments. The embodiments are examples, and any embodiment that has substantially the same configuration as the technical idea described in Claims of the present invention and exerts the same action and effect is included in the technical scope of the present invention.

1. A sulfonium salt monomer represented by the following general formula (a): 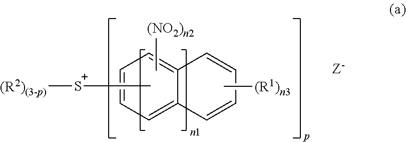
wherein p is 1, 2, or 3, n1 is 0 or 1, n2 is 1 or 2, and n3 is an integer of 0 to 6, provided that 1≤n2+n3≤5 is satisfied if n1 is 0, and 1≤n2+n3≤7 is satisfied if n1 is 1;
R¹ is a halogen atom, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein if n3 is 2 to 6, R¹ groups are identical to or different from each other, and any two R¹ groups are optionally bound together to form a ring together with the carbon atoms to which the two groups are bound;
R² is a halogen atom or a hydrocarbyl group having 1 to 30 carbon atoms and optionally containing a heteroatom, wherein if p is 1, the two R² groups are identical to or different from each other, and any two of the three substituents bound to S⁺ are optionally bound together to form a ring together with the sulfur atom to which the two substituents are bound; and
Z⁻ is a carboxylate anion having an aromatic vinyl structure and an iodine atom.

2. The sulfonium salt monomer according to claim 1, wherein the sulfonium salt monomer is represented by the following general formula (a1): 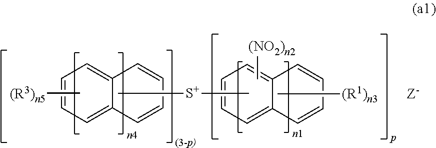
wherein p, n1 to n3, R¹, and Z⁻ are as specified above;
n4 is 0 or 1, and n5 is an integer of 0 to 5; and
R³ is a halogen atom, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein if n5 is 2 to 5, R³ groups are identical to or different from each other, and any two R³ groups are optionally bound together to form a ring together with the carbon atoms to which the two groups are bound.

3. The sulfonium salt monomer according to claim 1, wherein the Z⁻ is an anion represented by the following general formula (Z): 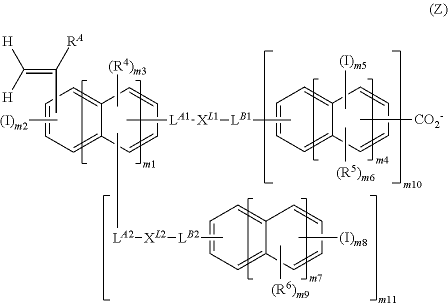
wherein m1 is 0 or 1, m2 is an integer of 0 to 4, m3 is an integer of 0 to 3, m4 is 0 or 1, m5 is an integer of 0 to 4, m6 is an integer of 0 to 3, m7 is 0 or 1, m8 is an integer of 0 to 4, m9 is an integer of 0 to 3, m10 is 0 or 1, and m11 is 0 or 1, provided that 0≤m2+m3+m11≤4 is satisfied if m1 is 0, and 0≤m2+m3+m11≤6 is satisfied if m1 is 1, that 0≤m5+m6≤4 is satisfied if m4 is 0, and 0≤m5+m6≤6 is satisfied if m4 is 1, that 0≤m8+m9≤5 is satisfied if m7 is 0, and 0≤m8+m9≤7 is satisfied if m7 is 1, and that 1≤m2+m5+m8≤4 is satisfied;
R^{A} is a hydrogen atom, a fluorine atom, a methyl group, or a trifluoromethyl group;
R⁴, R⁵, and R⁶ are each a halogen atom being not an iodine atom, a nitro group, a cyano group, a hydroxy group, a carboxy group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, wherein: if m3 is 2 or 3, R⁴ groups are identical to or different from each other, and any two R⁴ groups are optionally bound together to form a ring together with the carbon atoms to which the two groups are bound; if m6 is 2 or 3, R⁵ groups are identical to or different from each other, and any two R⁵ groups are optionally bound together to form a ring together with the carbon atoms to which the two groups are bound; and if m9 is 2 or 3, R⁶ groups are identical to or different from each other, and any two R⁶ groups are optionally bound together to form a ring together with the carbon atoms to which the two groups are bound;
L^{A1}, L^{A2}, L^{B1}, and L^{B2} are each independently a single bond, an ether bond, a carbonyl group, an ester bond, a sulfonate bond, an amide bond, a sulfonamide bond, a carbonate bond, or a carbamate bond; and
X^{L1} and X^{L2} are each independently a single bond or a hydrocarbylene group having 1 to 40 carbon atoms and optionally containing a heteroatom.

4. A sulfonium salt quencher comprising the sulfonium salt monomer according to claim 1.

5. A sulfonium salt quencher comprising the sulfonium salt monomer according to claim 2.

6. A sulfonium salt quencher comprising the sulfonium salt monomer according to claim 3.

7. A polymer comprising a repeating unit derived from the sulfonium salt quencher according to claim 4.

8. The polymer according to claim 7, wherein the polymer further comprises a repeating unit or repeating units represented by either one or both of the following general formulae (b1) and (b2): 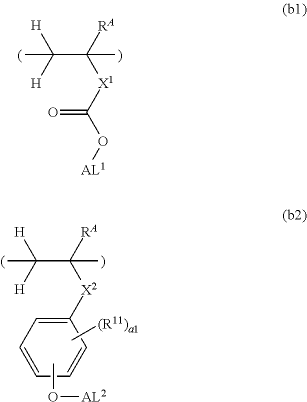
wherein R^{A} groups are each independently a hydrogen atom, a fluorine atom, a methyl group, or a trifluoromethyl group;
X¹ is a single bond, a phenylene group, a naphthylene group, *—C(═O)—O—X¹¹—, or *—C(═O)—NH—X¹¹—, wherein the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, X¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring;
X² is a single bond, *—C(═O)—O—, or *—C(═O)—NH—;
* indicates bonding to a carbon atom of a main chain;
R¹¹ is a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom, wherein if a1 is 2 or more, R¹¹ groups are identical to or different from each other;
AL¹ and AL² are each independently an acid-unstable group; and
a1 is an integer of 0 to 4.

9. The polymer according to claim 7, wherein the polymer further comprises a repeating unit represented by the following general formula (b3): 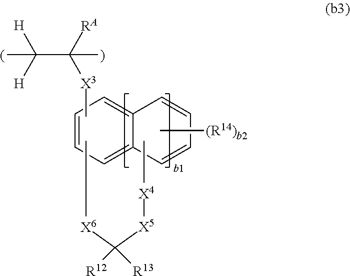
wherein b1 is 0 or 1, and b2 is an integer of 0 to 3 if b1 is 0, and an integer of 0 to 5 if b1 is 1;
R^{A} is a hydrogen atom, a fluorine atom, a methyl group, or a trifluoromethyl group;
X³ is a single bond, *—C(═O)—O—, or *—C(═O)—NH—, wherein * indicates bonding to a carbon atom of a main chain;
R¹² and R¹³ are each independently a hydrogen atom or a hydrocarbyl group having 1 to 20 carbon atoms and optionally containing a heteroatom, wherein R¹² and R¹³ are optionally bound together to form a ring together with the carbon atom to which the groups are bound;
R¹⁴ is a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A})(R^{14B}) wherein R^{14A} and R^{14B} are each independently a hydrogen atom or a hydrocarbyl group having 1 to 6 carbon atoms, and if b2 is 2 or more, R¹⁴ groups are identical to or different from each other, and any multiple R¹⁴ groups are optionally bound together to form a ring together with the carbon atoms of the aromatic ring or aromatic rings to which the groups are bound;
X⁴ is a single bond, an aliphatic hydrocarbylene group having 1 to 4 carbon atoms, a carbonyl group, a sulfonyl group, or a group given by combining any of these; and
X⁵ and X⁶ are each independently an oxygen atom or a sulfur atom, provided that X⁴ and X⁶ are bound to adjacent carbon atoms in the aromatic ring or aromatic rings.

10. The polymer according to claim 7, wherein the polymer further comprises a repeating unit represented by the following general formula (c): 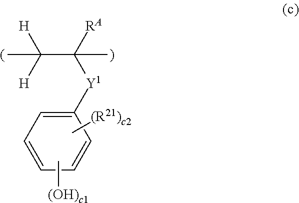
wherein R^{A} is a hydrogen atom, a fluorine atom, a methyl group, or a trifluoromethyl group;
Y¹ is a single bond, *—C(═O)—O—, or *—C(═O)—NH—, wherein * indicates bonding to a carbon atom of a main chain;
R²¹ is a halogen atom, a carboxy group, a nitro group, a cyano group, an alkoxycarbonyl group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
c1 is an integer of 1 to 4, and c2 is an integer of 0 to 3, provided that 1≤c1+c2≤5 is satisfied.

11. The polymer according to claim 7, wherein the polymer further comprises a repeating unit derived from an onium salt monomer containing a fluorosulfonate anion having a polymerizable group and at least one iodine atom and a sulfonium cation.

12. The polymer according to claim 7, wherein the polymer further comprises a repeating unit represented by the following general formula (e): 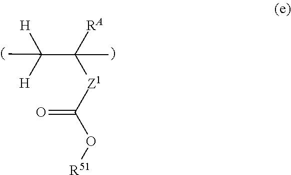
wherein R^{A} is a hydrogen atom, a fluorine atom, a methyl group, or a trifluoromethyl group;
Z¹ is a single bond, a phenylene group, a naphthylene group, *—C(═O)—O—Z¹¹—, or *—C(═O)—NH—Z¹¹—, wherein the phenylene group or naphthylene group is optionally substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, * indicates bonding to a carbon atom of a main chain, Z¹¹ is a saturated hydrocarbylene group having 1 to 10 carbon atoms, a phenylene group, or a naphthylene group, and the saturated hydrocarbylene group optionally contains a hydroxy group, an ether bond, an ester bond, or a lactone ring; and
R⁵¹ is a hydrogen atom or a group having 1 to 20 carbon atoms and containing at least one structure selected from a hydroxy group being not a phenolic hydroxy group, a cyano group, a carbonyl group, a carboxy group, an ether bond, an ester bond, a sulfonate bond, a carbonate bond, a lactone ring, a sultone ring, and a carboxylic anhydride (—C(═O)—O—C(═O)—).

13. A chemically amplified resist composition comprising (A) a base polymer containing the polymer according to claim 7.

14. The chemically amplified resist composition according to claim 13, wherein the chemically amplified resist composition further comprises one or more selected from (B) an organic solvent, (C) a quencher other than the sulfonium salt quencher, (D) an acid generator, and (E) a surfactant.

15. A patterning method comprising: a step of forming a resist film on a substrate by using the chemically amplified resist composition according to claim 13; a step of exposing the resist film to a high-energy ray; and a step of developing the exposed resist film by using a developer.

16. The patterning method according to claim 15, wherein ArF excimer laser light having a wavelength of 193 nm, KrF excimer laser light having a wavelength of 248 nm, an electron beam, or an extreme ultraviolet ray having a wavelength of 3 to 15 nm is used as the high-energy ray.
